(19) United States
(12) Patent Application Publication
(10) Pub. No.: US 2026/0008760 A1
(43) Pub. Date: Jan. 8, 2026

(54) DIPEPTIDYL PEPTIDASE 1 INHIBITORS AND USES THEREOF

(71) Applicant: Insmed Incorporated, Bridgewater, NJ (US)

(72) Inventors: Adam J. PLAUNT, Bridgewater, NJ (US); Robert STAUBACH, Bridgewater, NJ (US); Gianpaolo GOBBO, Bridgewater, NJ (US); Byungchan KIM, Bridgewater, NJ (US); Yingxin SHI, Bridgewater, NJ (US)

(21) Appl. No.: 19/325,182

(22) Filed: Sep. 10, 2025

Related U.S. Application Data
(63) Continuation of application No. PCT/CN2025/082555, filed on Mar. 14, 2025.

(30) Foreign Application Priority Data
Apr. 12, 2024 (WO) PCT/CN2024/087534

(51) Int. Cl. C07D 267/22

(52) U.S. Cl. CPC C07D 267/22

(57) ABSTRACT

Provided herein are compounds of formulae (I)-(IX), or pharmaceutically acceptable salts or deuterated forms thereof as defined herein. Also provided herein are pharmaceutical compositions comprising a compound of formula (I)-(IX) or pharmaceutically acceptable salt or deuterated form thereof, and methods of using a compound of formula (I)-(IX) or pharmaceutically acceptable salt or deuterated form thereof, e.g., in the treatment of a disease that is treatable by administration of a DPP1 inhibitor.

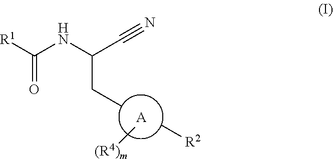

### CROSS REFERENCE TO RELATED APPLICATIONS

This application claims priority to International Patent Application No. PCT/CN2024/087534, filed on Apr. 12, 2024, the disclosure of which is incorporated by reference herein in its entirety.

### BACKGROUND

Dipeptidyl peptidase 1 (DPP1; EC 3.4.14.1), also known as cathepsin C, is a lysosomal cysteine protease belonging to the papain family having a molecular weight of 200 kDa. DPP1 was first discovered by Gutman and Fruton in 1948 (J Biol Chem, 174, 851-858); however, the cDNA of the human enzyme was first described in 1995 (Paris et al. 1995, FEBS Lett, 369, 326-330). DPP1 is the only member of the papain family that is functional as a tetramer, consisting of four identical subunits. Each subunit is composed of an N-terminal fragment, a heavy chain and a light chain (Dolenc et al. 1995, J Biol Chem, 270, 21626-21631).

DPP1 is constitutively expressed in many tissues with highest levels in lung, kidney, liver and spleen. DPP1 catalyzes the removal of dipeptides from the N-terminal end of polypeptide substrates with broad specificity. Recent data suggest that besides being an important enzyme in lysosomal protein degradation, DPP1 also functions as a key enzyme in the activation of granule serine proteases in cytotoxic T-lymphocytes and natural killer cells (granzymes A and B), mast cells (chymase and tryptase) and neutrophils (cathepsin G, neutrophil elastase and proteinase-3).

Mast cells are found in many tissues but are present in greater numbers along the epithelial linings of the body, such as the skin, respiratory tract and gastrointestinal tract. In humans, two types of mast cells have been identified. The T-type, which expresses only tryptase, and the MC-type, which expresses both tryptase and chymase. In humans, the T-type mast cells are located primarily in alveolar tissue and intestinal mucosa while the TC-type cells predominate in skin and conjunctiva. Tryptase and chymase appear to be important mediators of allergic diseases, being involved in processes of inflammation, bronchoconstriction and mucus secretion.

Neutrophils play a critical role in host defense against invading pathogens. Neutrophils are produced in the bone marrow and are fully mature when released into the circulation to take up their role as the first line of cellular defense. Pro-inflammatory mediators and chemotactic attractants activate neutrophils and draw them to the site of infection, where they act to engulf bacteria by phagocytosis, assaulting them with an arsenal of anti-bacterial compounds that use both oxidative and non-oxidative methods of attack. The powerful serine protease, neutrophil elastase, is one of those anti-bacterial compounds that are clearly involved in destroying bacteria. Neutrophil elastase is released into the phagolysome surrounding the microorganism, which it proceeds to destroy. Neutrophil elastase is able to attack the outer membrane protein, OmpA, in gram-negative bacteria, helping to directly kill the pathogen by degrading its membrane, as well as enabling other anti-bacterial compounds to gain access to the pathogen. In addition, neutrophil elastase may help process other antibacterial compounds, converting them from inactive pro-peptides into their active states, such as for cathelicidin.

Yet neutrophil elastase can also cause problems for its host. It is one of the most destructive enzymes in the body, with the capability of degrading extracellular matrix proteins (including collagens, proteoglycan, fibronectin, platelet receptors, complement receptor, thrombomodulin, lung surfactant and cadherins) and key plasma proteins (including coagulation and complement factors, immunoglobulin, several proteases and protease inhibitors). Under physiological conditions, endogenous protease inhibitors, such as al-antitrypsin, tightly regulate the activity of neutrophil elastase. However, at inflammatory sites, neutrophil elastase is able to evade regulation, and once unregulated it can induce the release of pro-inflammatory cytokines, such as interleukin-6 and interleukin-8, leading to acute lung injury. It can even impair host defense against infection by degrading phagocyte surface receptors and opsonins. Its negative role has been reported in a number of diseases characterized by tissue destruction and inflammation.

As such, there is a need in the art to provide novel DPP1 inhibitors in order to treat the aforementioned diseases, and others associated with DPP1 and neutrophil elastase.

### SUMMARY

In embodiments, the present disclosure provides a compound of formula (I):

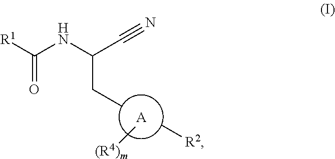

or a pharmaceutically acceptable salt, a stereoisomer, or a deuterated form thereof, wherein:
ring A is a carbocyclyl, aryl, heterocyclyl, or heteroaryl ring;
R¹ is

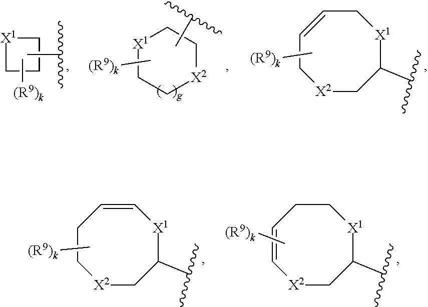

or C₁-C₆ alkylene(NH)-carbocycle substituted with 0, 1, 2, 3, or 4 R³;
R² is

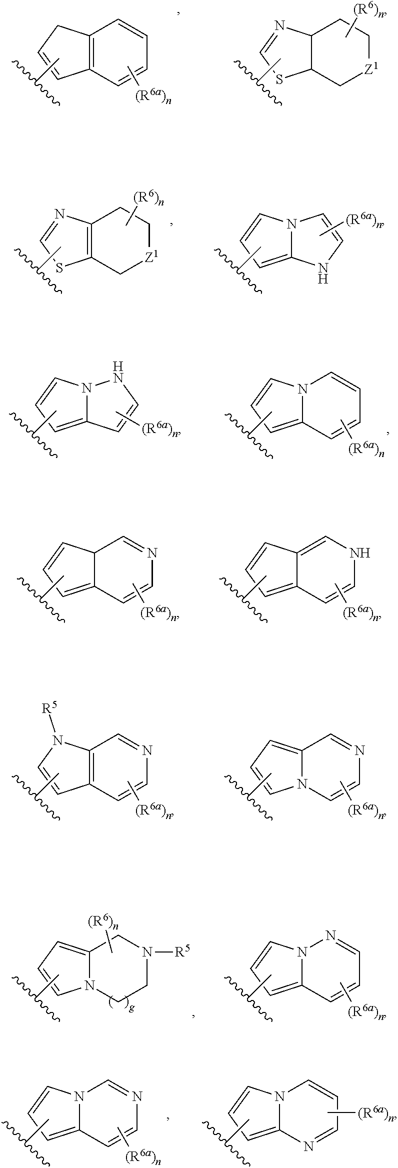

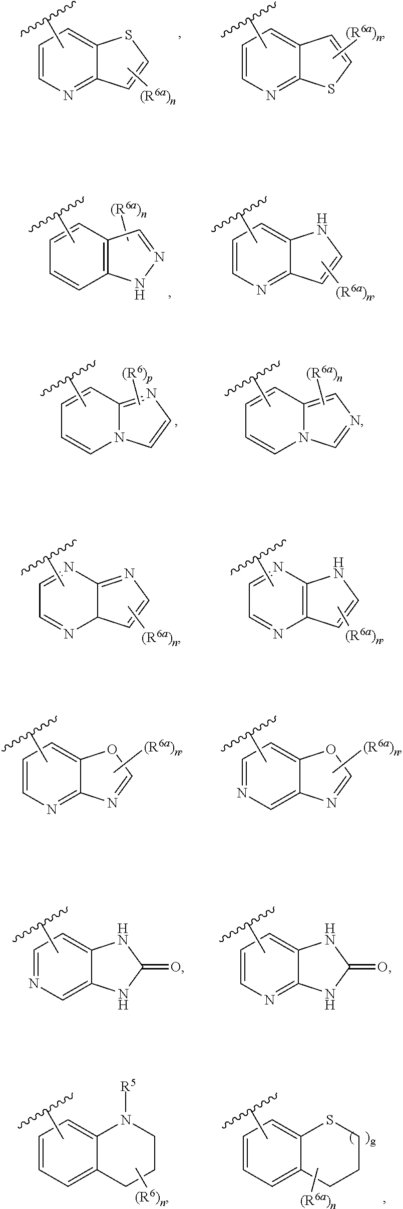

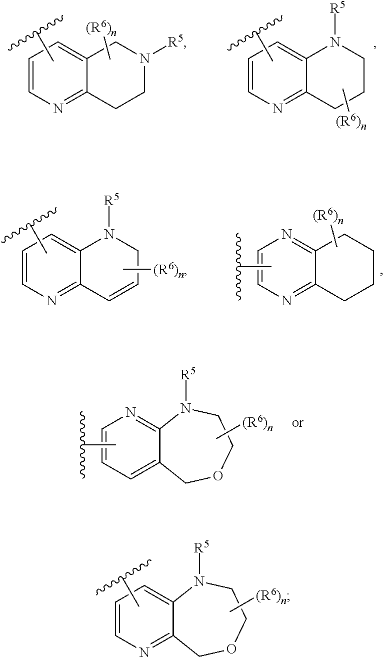

each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R^{6a} is each independently halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
R¹¹ and R¹² are each independently hydrogen, halogen, or —C₁-C₆ alkyl;
Z¹ is CH₂, NR⁵, S, S(O), or S(O)₂;
m is 0, 1, 2, or 3;
n is 0, 1, 2, 3, or 4;
each g is independently 0, 1, 2, or 3; and
k is 0, 1, 2, 3, or 4;
provided that when R² is

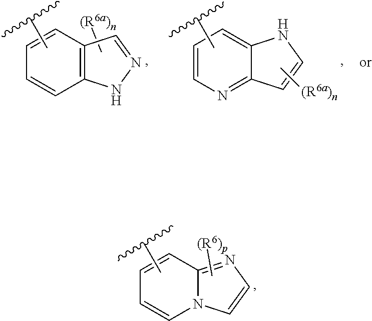

then A is not optionally substituted phenylene.

In embodiments, the present disclosure provides a compound of formula (II):

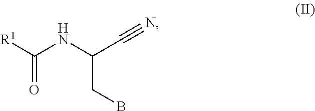

or a pharmaceutically acceptable salt, a stereoisomer, or a deuterated form thereof, wherein:
B is

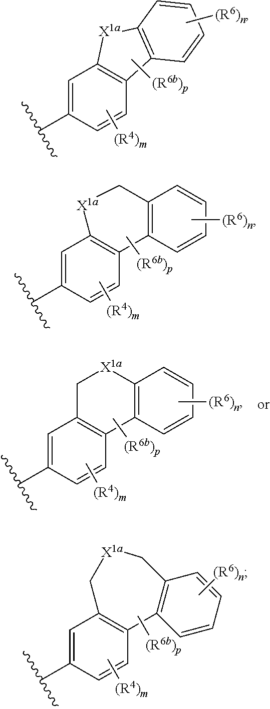

X^{1a} is —NR⁵—, —O—, —CR¹¹R¹²—, —C(O)—, —S—, —S(O)— or —S(O)₂—;
R¹ is

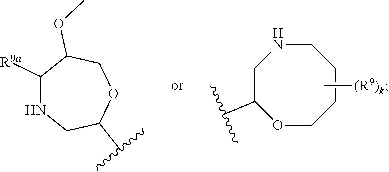

each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is each independently, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R^{6b} is each independently, halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
R^{9a} is C₁-C₃ alkyl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
k is 0, 1, 2, 3, or 4;
m is 0, 1, 2, or 3;
n is 0, 1, 2, or 3; and
p is 0, 1, or 2.

In embodiments, the present disclosure provides a compound of formula (III):

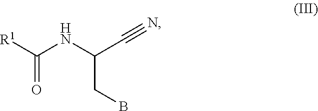

or a pharmaceutically acceptable salt, a stereoisomer, or a deuterated form thereof, wherein:
B is

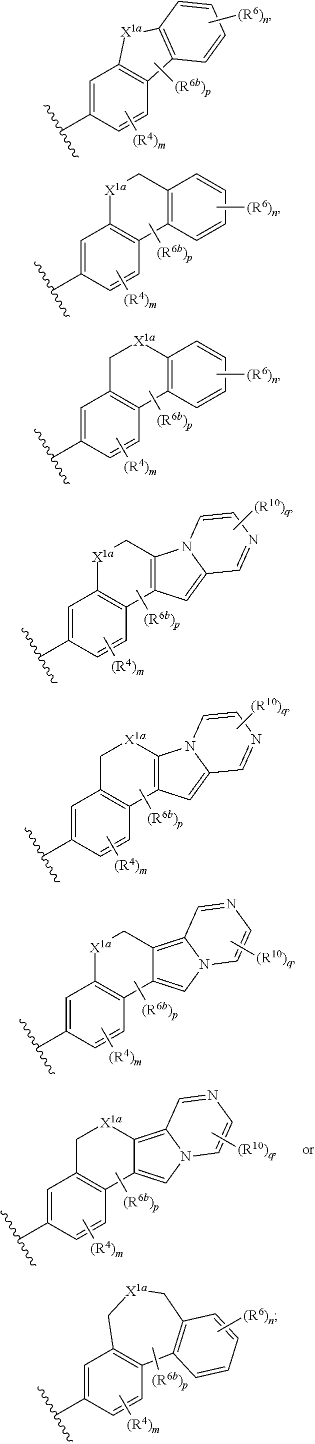

X^{1a} is —NR⁵—, —O—, —CR¹¹R¹²—, —C(O)—, —S—, —S(O)— or —S(O)₂—;
R¹ is

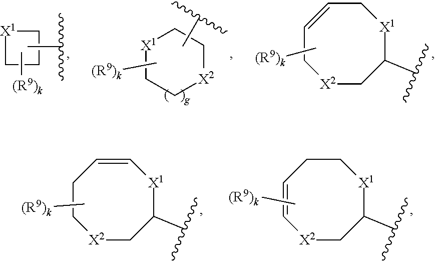

or C₁-C₆ alkylene(NH)-carbocycle substituted with 0, 1, 2, 3, or 4 R³;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
two R⁶ taken together with the atoms to which they are attached forms a monocyclic or bicyclic carbocyclyl, aryl, heteroaryl, or heterocyclyl, each optionally substituted by 1 or 2 R¹⁰;
each R^{6b} is each independently, halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
g is 0, 1, 2, or 3;
k is 0, 1, 2, 3, or 4;
m is 0, 1, 2, or 3;
n is 2; and
p is 0, 1, or 2; and
q is 0, 1, or 2.

In embodiments, the present disclosure provides a compound of formula (IV):

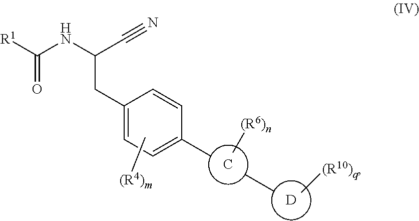

or a pharmaceutically acceptable salt, a stereoisomer, or a deuterated form thereof, wherein:
ring C is a monocyclic heteroaryl ring;
ring D is a monocyclic heteroaryl or a monocyclic heterocyclyl ring;
R¹ is

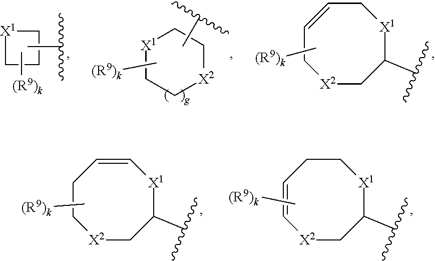

or C₁-C₆ alkylene(NH)-carbocycle substituted with 0, 1, 2, 3, or 4 R³;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
g is 0, 1, 2, or 3;
k is 0, 1, 2, 3, or 4;
m is 0, 1, 2, or 3;
n is 0, 1, 2, 3, or 4; and
q is 0, 1, 2, or 3.

In embodiments, the present disclosure provides a compound of formula (V):

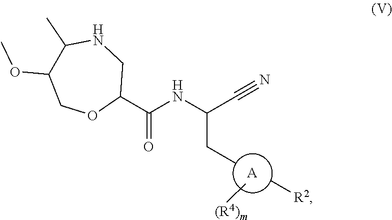

or a pharmaceutically acceptable salt, a stereoisomer, or a deuterated form thereof, wherein:
ring A is a carbocycle, aryl, heterocycle, or heteroaryl ring;
R² is monocyclic, bicyclic, or tricyclic ring, wherein R² is optionally substituted with 1, 2, 3, or 4 R⁵, and optionally substituted with 1, 2, 3, or 4 R⁶, wherein each R⁵ is bound to a ring nitrogen;
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, or —(C₁-C₆ alkylene)-O—(C₁-C₆ alkyl), or R⁷ and R⁸ together with the nitrogen atom to which they are attached form a heterocyclyl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle; and
m is 0, 1, 2, or 3.

In embodiments, the present disclosure provides a compound of formula (VI):

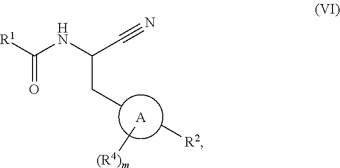

or a pharmaceutically acceptable salt, a stereoisomer, or a deuterated form thereof, wherein:
ring A is a carbocyclyl, aryl, heterocyclyl, or heteroaryl ring;
R¹ is

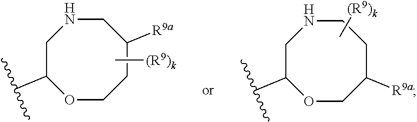

R² is monocyclic, bicyclic, or tricyclic ring, wherein R² is optionally substituted with 1, 2, 3, or 4 R⁵, and optionally substituted with 1, 2, 3, or 4 R⁶, wherein each R⁵ is bound to a ring nitrogen;
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
R^{9a}—O—(C₁-C₆ alkyl);
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
m is 0, 1, 2, or 3;
n is 0, 1, 2, 3, or 4;
each g is independently 0, 1, 2, or 3; and
k is 0, 1, 2, or 3;
wherein the compound is not a compound disclosed in WO2024/148308.

In embodiments, the present disclosure provides a compound of formula (VII):

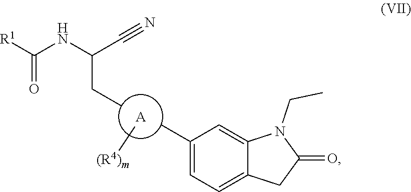

or a pharmaceutically acceptable salt, a stereoisomer, or a deuterated form thereof, wherein:
ring A is a carbocyclyl, aryl, heterocyclyl, or heteroaryl ring;
R¹ is

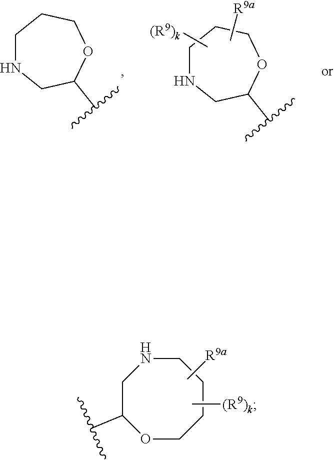

each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁹ is independently halogen, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, or —S(═NH)(O)(C₁-C₆ alkyl;
R^{9a} is —OH or —OC₁-C₃ alkyl;
m is 0, 1, or 2; and
k is 0, 1, 2, 3, or 4.

In embodiments, the present disclosure provides a compound of formula (VIII):

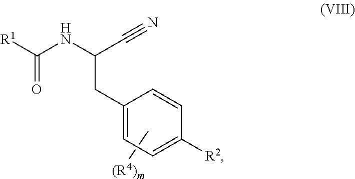

or a pharmaceutically acceptable salt, a stereoisomer, or a deuterated form thereof, wherein:
R¹ is

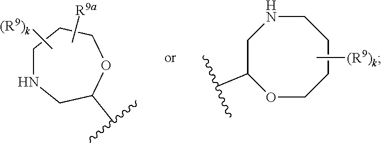

R² is

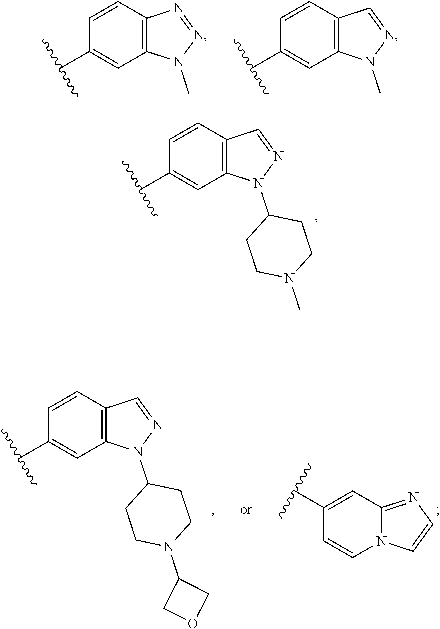

each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁹ is independently halogen, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, or —S(═NH)(O)(C₁-C₆ alkyl;
R^{9a} is —OH or —OC₁-C₃ alkyl;
m is 0, 1, or 2; and
k is 0, 1, 2, 3, or 4.

In embodiments, the present disclosure provides a compound of formula (IX):

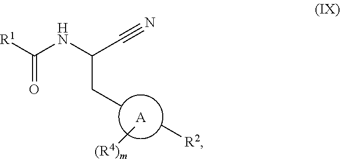

or a pharmaceutically acceptable salt, a stereoisomer, or a deuterated form thereof, wherein:
ring A is a carbocyclyl, aryl, heterocyclyl, or heteroaryl ring;
R¹ is

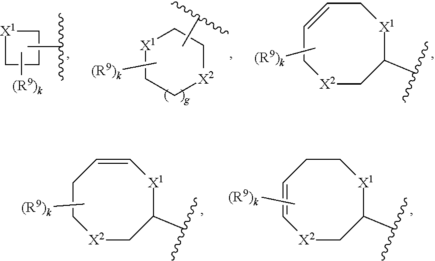

C₁-C₆ alkylene(NH)-carbocycle substituted with 0, 1, 2, 3, or 4 R³;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
R² is

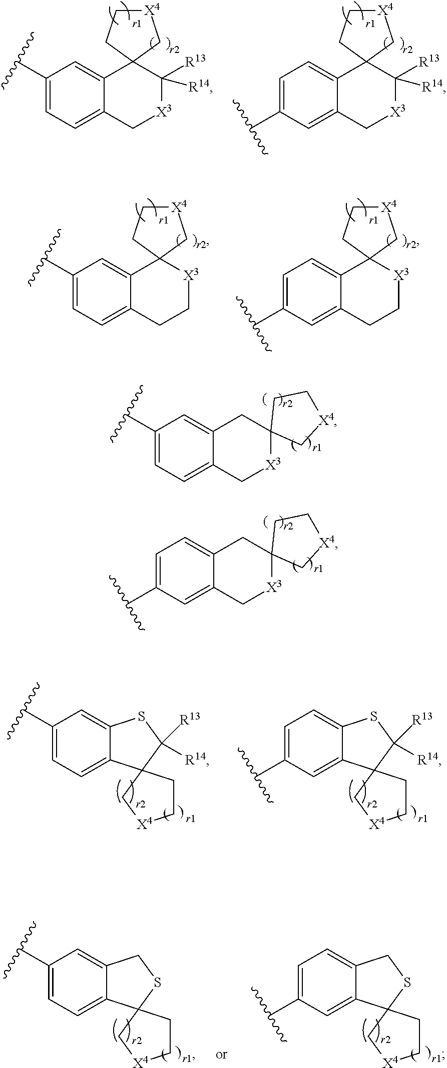

X³ is —O—, —S—, —NH—, —N(C₁-C₆ alkyl)-, —N(C₁-C₆ haloalkyl)-, —N(C(O)(C₁-C₆ alkyl))-, or —CR¹¹R¹²—;
X⁴ is —O—, —S—, —NH—, —N(CN)—, —N(C₁-C₆ alkyl)-, —N(C₁-C₆ haloalkyl)-, —N(C(O)(C₁-C₆ alkyl))-, —N(carbocycle)-, —N(heterocycle)-, or —N(heteroaryl)-, wherein each carbocycle, heterocyclyl, and heteroaryl is optionally substituted by 1 or 2 R¹⁰;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
R¹³ and R¹⁴ are each H or R¹³ and R¹⁴ together forms an oxo (═O);
g is 0, 1, 2, or 3;
k is 0, 1, 2, 3, or 4;
m is 0, 1, 2, or 3;
n is 0, 1, 2, 3, or 4;
r1 is 1 or 2; and
r2 is 1 or 2.

In embodiments, the present disclosure provides a compound selected from Compounds A4-A13, A15-A141, or A145-A172, or a pharmaceutically acceptable salt, a stereoisomer, a racemic form thereof, or a deuterated form thereof.

In embodiments, the present disclosure provides a compound selected from Compounds B5-B30, or a pharmaceutically acceptable salt, a stereoisomer, a racemic form thereof, or a deuterated form thereof.

In embodiments, the present disclosure provides a compound selected from Compounds C1-C34, or a pharmaceutically acceptable salt, a stereoisomer, a racemic form thereof, or a deuterated form thereof.

In embodiments, the present disclosure provides a compound selected from Compounds D1-D6, D8, or D9, or a pharmaceutically acceptable salt, a stereoisomer, a racemic form thereof, or a deuterated form thereof.

In embodiments, the present disclosure provides a compound selected from Compounds E10, E12, E14, or E16, or a pharmaceutically acceptable salt, a stereoisomer, a racemic form thereof, or a deuterated form thereof.

In embodiments, the present disclosure provides a compound selected from Compounds F1-F30, or a pharmaceutically acceptable salt, a stereoisomer, a racemic form thereof, or a deuterated form thereof.

In embodiments, the present disclosure provides a compound selected from Compounds 32 or G1-G12, or a pharmaceutically acceptable salt, a stereoisomer, a racemic form thereof, or a deuterated form thereof.

In embodiments, the present disclosure provides a compound selected from Compounds H1-H44, or a pharmaceutically acceptable salt, a stereoisomer, a racemic form thereof, or a deuterated form thereof.

In embodiments, the present disclosure provides a compound selected from Compounds I1-I20, or a pharmaceutically acceptable salt, a stereoisomer, a racemic form thereof, or a deuterated form thereof.

In embodiments, the present disclosure provides a compound selected from Compounds A1-A3, A14, A142-A144, B1, B3, B4, B31-B35, D7, 1-5, 7-11, or 18-35, or a pharmaceutically acceptable salt, a stereoisomer, a racemic form thereof, or a deuterated form thereof.

In embodiments, the present disclosure provides a compound selected from Compounds B2, 6, or 12-14, or a pharmaceutically acceptable salt or a deuterated form thereof.

In embodiments, the present disclosure provides a pharmaceutical composition comprising a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof), and a pharmaceutically acceptable adjuvant, diluent or carrier.

In embodiments, the present disclosure provides a method for treating an obstructive disease of the airway in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating cystic fibrosis in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating chronic chinosinusitis in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating hidradenitis suppurativa in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating cancer in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating lupus nephritis in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating arthritis in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating inflammatory bowel disease in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating an anti-neutrophil cytoplasmic antibody associated vasculitis in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating a disease in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof), wherein the disease is giant cell arteritis, polyarteritis nodosa, anti-GBM disease (Goodpasture's), systemic scleroderma, diabetic nephropathy, diabetic neuropathy, diabetic retinopathy, diabetic ulcers, Duchenne muscular dystrophy, bronchiolitis obliterans, atopic dermatitis, pyoderma gangrenosum, sweet's syndrome, dermatomyositis/polymyositis, neutrophilic dermatoses, thrombosis, bronchopulmonary dysplasia, amyotrophic lateral sclerosis, sickle cell anemia, psoriasis, or a ventilator-induced lung injury.

In embodiments, the present disclosure provides a method for treating heart failure in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof).

In embodiments, the present disclosure provides a method for treating ischemia/reperfusion (IR) injury in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof) or a pharmaceutical composition comprising the compound disclosed herein.

In embodiments, the present disclosure provides a method for treating liver injury in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof) or a pharmaceutical composition comprising the compound disclosed herein.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

Listed below are definitions of various terms used in the specification and claims to describe the present disclosure.

Unless defined otherwise, all technical and scientific terms used in this disclosure have the same meanings as commonly understood by one of ordinary skill in the art to which this disclosure belongs.

The term “about” when immediately preceding a numerical value means a range encompassing said numerical value plus or minus an acceptable amount of variation in the art (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkynyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon ring atoms and at least one aromatic ring. For purposes of this disclosure, the aryl radical can be a monocyclic, bicyclic, tricyclic or tetracyclic ring system, which can include fused, bridged, or spiro ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In embodiments where “L” is aryl, the aryl radical is a diradical. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R, is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiro ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiro ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiro ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl” “heterocyclic ring” or “heterocycle” refers to a stable 3- to 20-membered non-aromatic, saturated or partially unsaturated ring radical which consists of two to twelve carbon ring atoms and from one to six heteroatoms as ring atoms selected from nitrogen, oxygen or sulfur, at least one non-aromatic, saturated or partially unsaturated ring containing at least one heteroatom as a ring atom. Unless stated otherwise specifically in the specification, the heterocyclyl radical can be a monocyclic, bicyclic, tricyclic or tetracyclic ring system, which can include fused, bridged, or spiro ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In embodiments where “L” is heterocyclyl, the heterocyclyl radical is a diradical. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocycloalkyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising one to thirteen carbon ring atoms, one to six heteroatoms as ring atoms selected from nitrogen, oxygen and sulfur, and at least one aromatic ring containing at least one heteroatom as a ring atom. For purposes of this disclosure, the heteroaryl radical can be a monocyclic, bicyclic, tricyclic or tetracyclic ring system, which can include fused, bridged, or spiro ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). In embodiments where “L” is heteroaryl, the heteroaryl radical is a diradical. Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

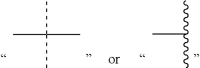

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

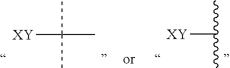

indicates that the chemical entity “XY” is bonded to another chemical entity via the point of attachment bond. Furthermore, the specific point of attachment to the non-depicted chemical entity can be specified by inference.

In this specification, unless stated otherwise, the term “pharmaceutically acceptable” is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

The term “treating” as used herein with regard to a patient, refers to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

An “effective amount” means the amount compound or pharmaceutical formulation, that when administered to a patient for treating a state, disorder or condition is sufficient to effect such treatment.

The term “therapeutically effective” applied to dose or amount refers to that quantity of a compound or pharmaceutical formulation that is sufficient to result in a desired clinical benefit after administration to a patient in need thereof. A “therapeutically effective amount”, in some embodiments, is a dose or amount of a compound or pharmaceutical formulation that is sufficient to result in prophylaxis after administration to a patient in need thereof.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human.

### Compounds

In one aspect of the present disclosure, a DPP1 inhibitor is provided, and the DPP1 inhibitor is a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof.

In embodiments, the present disclosure provides a compound of formula (I):

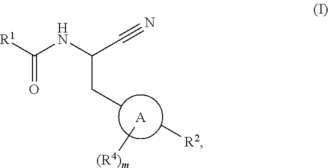

or a pharmaceutically acceptable salt, a stereoisomer, or a deuterated form thereof, wherein:
ring A is a carbocyclyl, aryl, heterocyclyl, or heteroaryl ring;
R¹ is

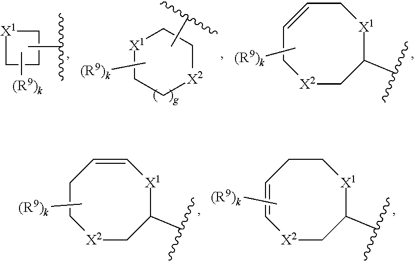

or C₁-C₆ alkylene(NH)-carbocycle substituted with 0, 1, 2, 3, or 4 R³;
R² is

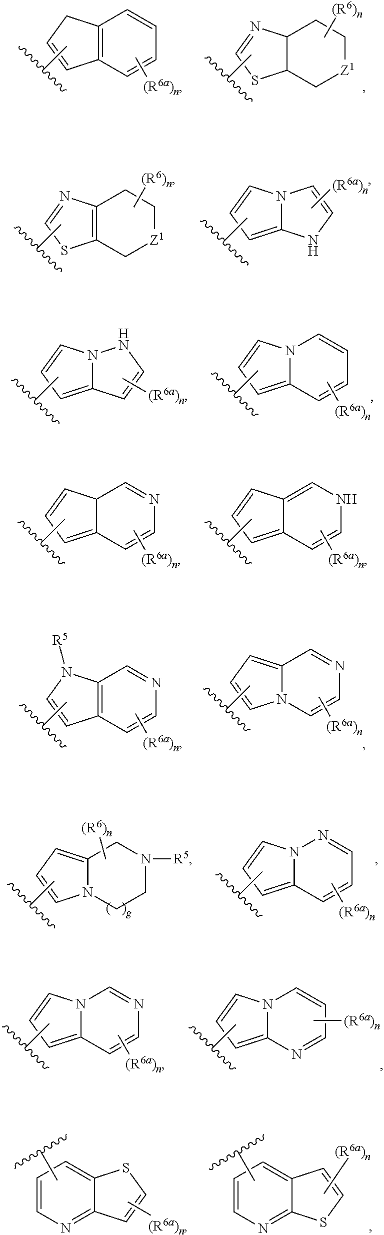

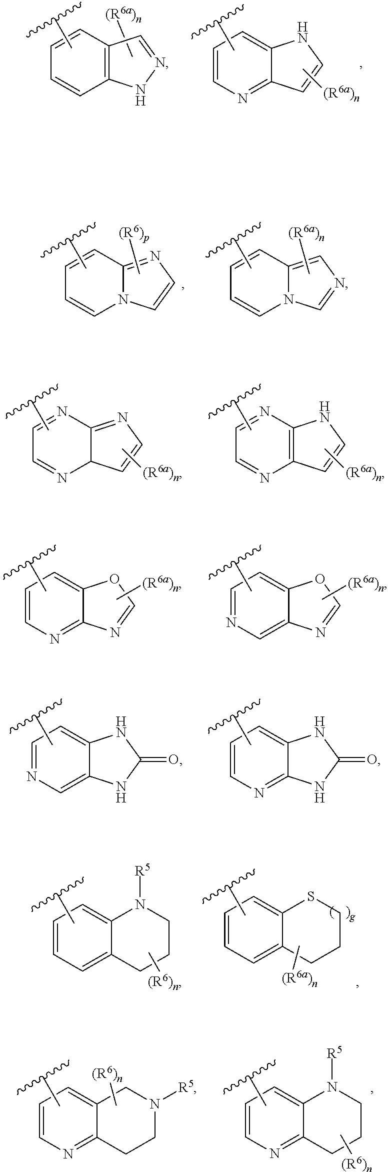

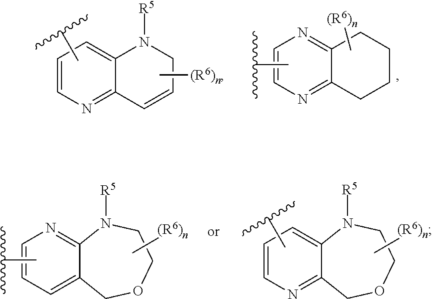

each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R^{6a} is each independently halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
R¹¹ and R¹² are each independently hydrogen, halogen, or —C₁-C₆ alkyl;
Z¹ is CH₂, NR⁵, S, S(O), or S(O)₂;
m is 0, 1, 2, or 3;
n is 0, 1, 2, 3, or 4;
each g is independently 0, 1, 2, or 3; and
k is 0, 1, 2, 3, or 4;
provided that when R² is

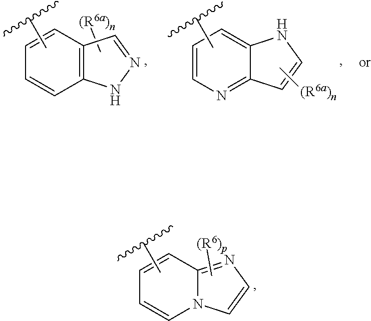

then A is not optionally substituted phenylene.

In embodiments, the present disclosure provides a compound of formula (I′):

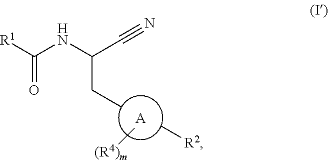

or a pharmaceutically acceptable salt, a stereoisomer, or a deuterated form thereof, wherein:
ring A is a carbocyclyl, aryl, heterocyclyl, or heteroaryl ring;
R¹ is

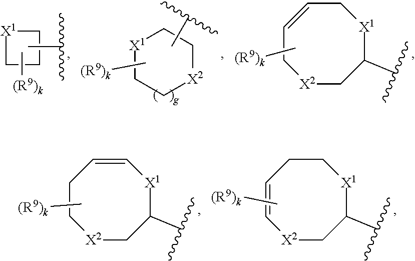

or C₁-C₆ alkylene(NH)-carbocycle substituted with 0, 1, 2, 3, or 4 R³;
R² is

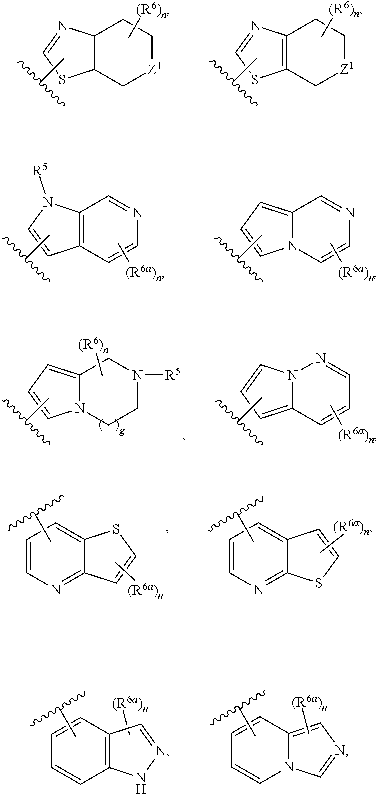

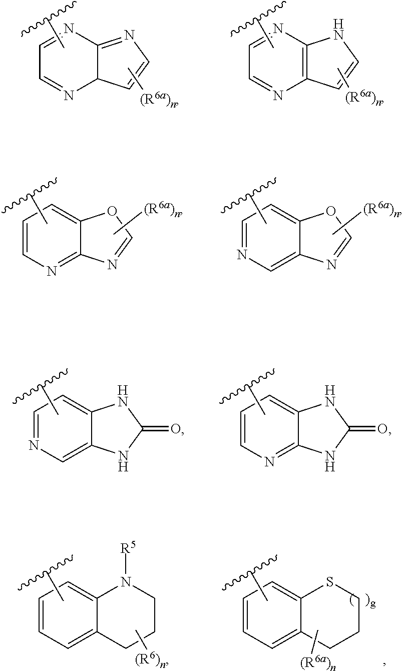

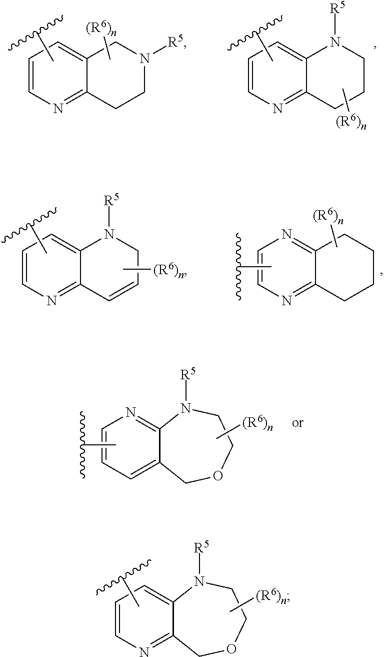

each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R^{6a} is each independently halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
R¹¹ and R¹² are each independently hydrogen, halogen, or —C₁-C₆ alkyl;
Z¹ is CH₂, NR⁵, S, S(O), or S(O)₂;
m is 0, 1, 2, or 3;
n is 0, 1, 2, 3, or 4;
each g is independently 0, 1, 2, or 3; and
k is 0, 1, 2, 3, or 4;
provided that when R² is

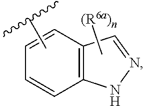

then A is not optionally substituted phenylene.

In embodiments of the compounds of formula (I), or a pharmaceutically acceptable salt, a stereoisomer, or a deuterated form thereof, R² is

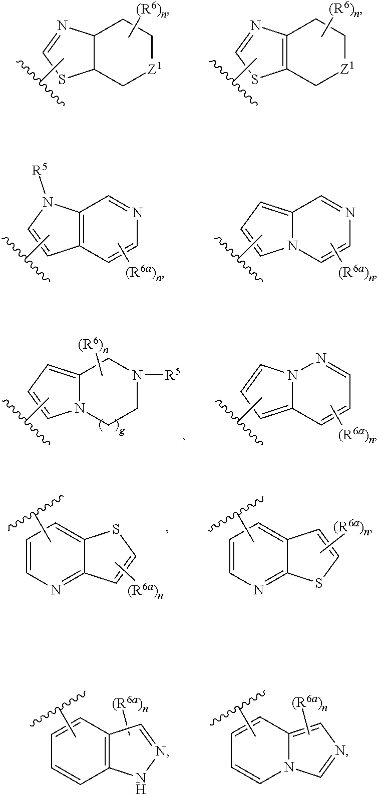

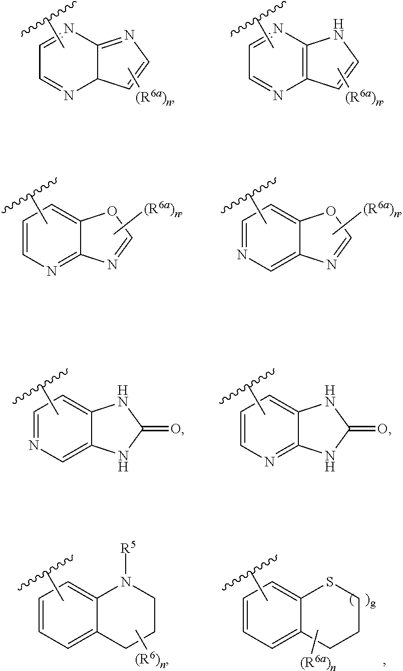

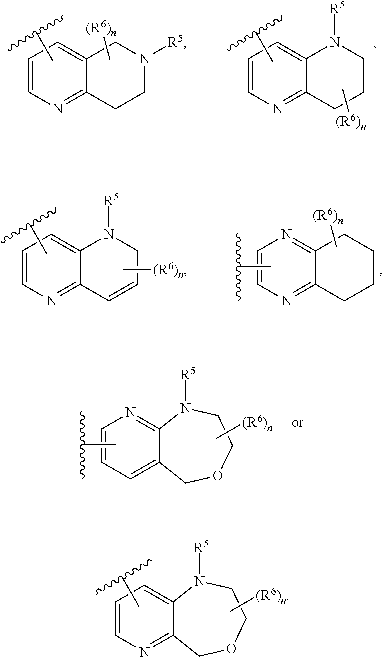

In embodiments of the compounds of formula (I), or a pharmaceutically acceptable salt, a stereoisomer, or a deuterated form thereof, R² is

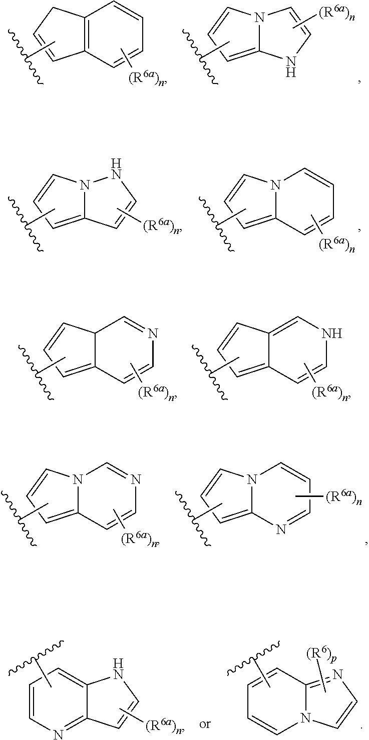

In embodiments of the compounds of formula (I), or a pharmaceutically acceptable salt, a stereoisomer, or a deuterated form thereof, R² is

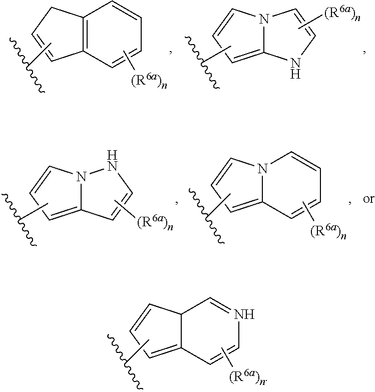

In embodiments, n is 1 or 2. In embodiments, R⁶ is each independently halogen, —OH, oxo, —CN, or —C₁-C₆ alkyl. In embodiments, R⁶ is each independently halogen or methyl.

In embodiments of the compounds of formula (I), or a pharmaceutically acceptable salt, a stereoisomer, or a deuterated form thereof, R² is

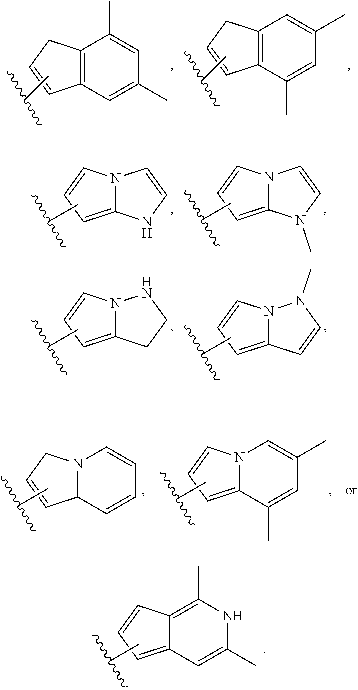

In embodiments, R² is

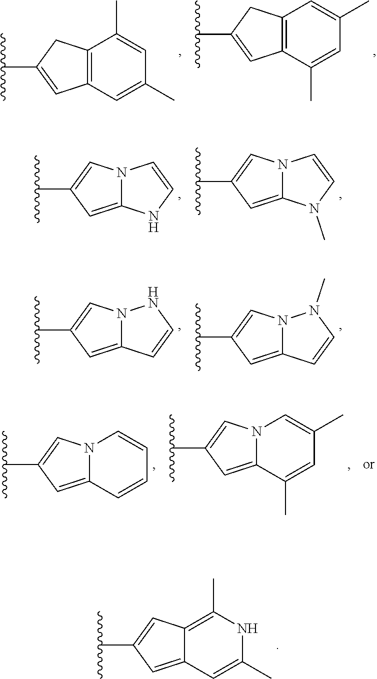

In embodiments of the compounds of formula (I), or a pharmaceutically acceptable salt, a stereoisomer, or a deuterated form thereof, R² is

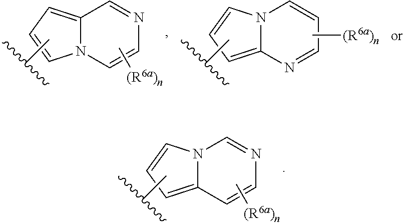

In embodiments, n is 1 or 2. In embodiments, R⁶ is each independently halogen, —OH, oxo, —CN, or —C₁-C₆ alkyl. In embodiments, R⁶ is each independently halogen or methyl.

In embodiments of the compounds of formula (I) or (I′), or a pharmaceutically acceptable salt, a stereoisomer, or a deuterated form thereof, R² is

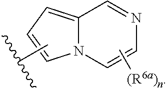

In embodiments, n is 1 or 2. In embodiments, R⁶ is each independently halogen, —OH, oxo, —CN, or —C₁-C₆ alkyl. In embodiments, R⁶ is each independently halogen or methyl.

In embodiments of the compounds of formula (I), or a pharmaceutically acceptable salt, a stereoisomer, or a deuterated form thereof, R² is

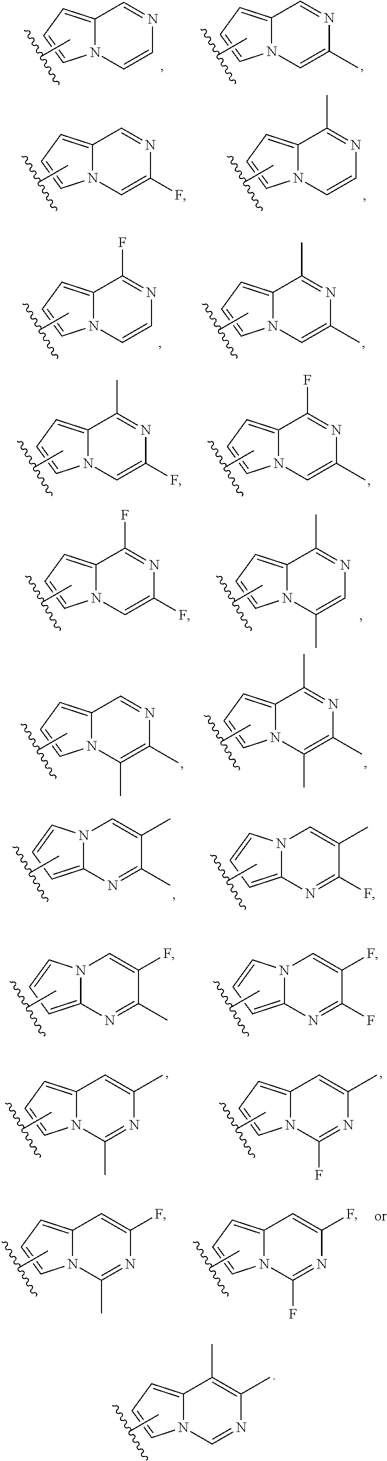

In embodiments, R² is

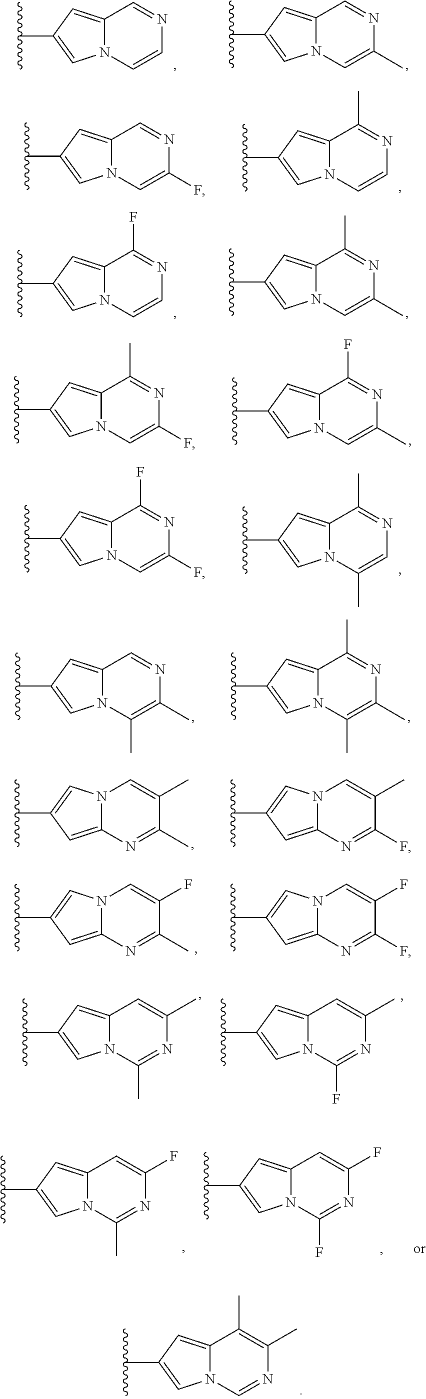

In embodiments of the compounds of formula (I) or (I′), or a pharmaceutically acceptable salt, a stereoisomer, or a deuterated form thereof, R² is

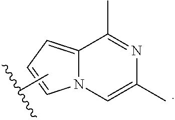

In embodiments, R² is

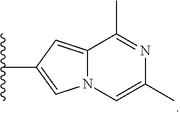 [SMILES: *c1cc2c(C)nc(C)cn2c1]

In embodiments of the compounds of formula (I), or a pharmaceutically acceptable salt, a stereoisomer, or a deuterated form thereof, R² is

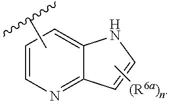

In embodiments, n is 1 or 2. In embodiments, R⁶ is each independently halogen, —OH, oxo, —CN, or —C₁-C₆ alkyl. In embodiments, R⁶ is each independently halogen or methyl.

In embodiments of the compounds of formula (I), or a pharmaceutically acceptable salt, a stereoisomer, or a deuterated form thereof, R² is

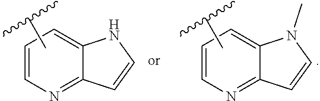

In embodiments, R² is

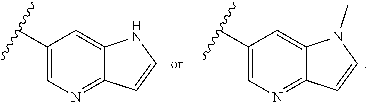 [SMILES: *c1cnc2cc[nH]c2c1 ; *c1cnc2ccn(C)c2c1]

In embodiments of the compounds of formula (I), or a pharmaceutically acceptable salt, a stereoisomer, or a deuterated form thereof, R² is

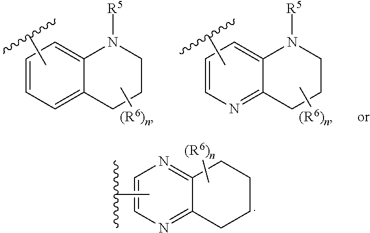

In embodiments, R⁵ is hydrogen, methyl, or —CONH₂. In embodiments, n is 0 or 1. In embodiments, R⁶ is oxo.

In embodiments of the compounds of formula (I) or (I′), or a pharmaceutically acceptable salt, a stereoisomer, or a deuterated form thereof, R² is

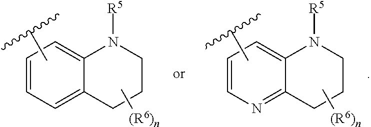

In embodiments, R⁵ is hydrogen, methyl, or —CONH₂. In embodiments, n is 0 or 1. In embodiments, R⁶ is oxo.

In embodiments of the compounds of formula (I) or (I′), or a pharmaceutically acceptable salt, a stereoisomer, or a deuterated form thereof, R² is

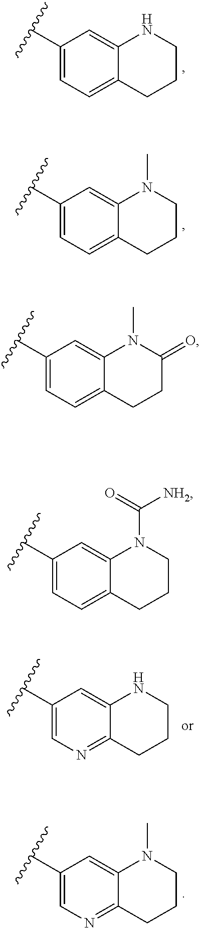 [SMILES: *c1ccc2c(c1)NCCC2 ; *c1ccc2c(c1)N(C)CCC2 ; *c1ccc2c(c1)N(C)C(=O)CC2 ; *c1ccc2c(c1)N(C(N)=O)CCC2 ; *c1cnc2c(c1)NCCC2 ; *c1cnc2c(c1)N(C)CCC2]

In embodiments, R² is

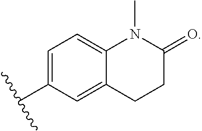 [SMILES: *c1ccc2c(c1)CCC(=O)N2C]

In embodiments, R² is

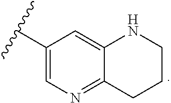 [SMILES: *c1cnc2c(c1)NCCC2]

In embodiments of the compounds of formula (I), or a pharmaceutically acceptable salt, a stereoisomer, or a deuterated form thereof, R² is

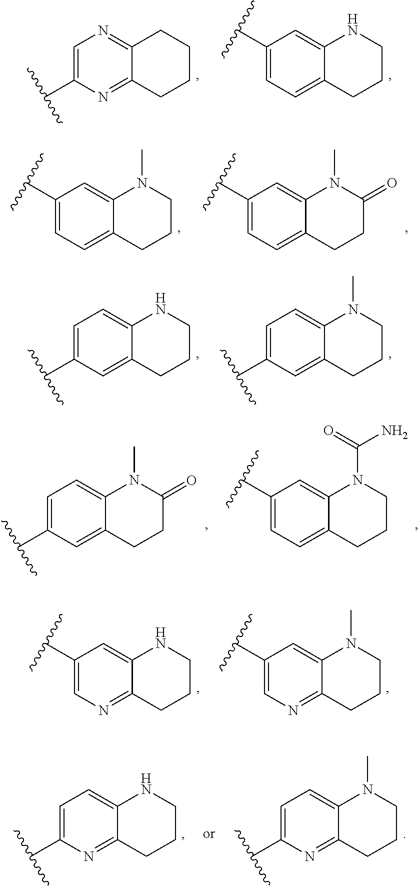

In embodiments, R² is

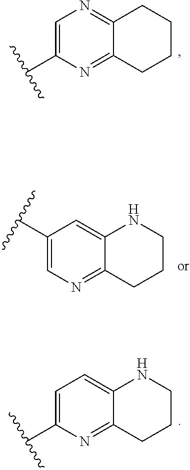 [SMILES: *c1cnc2c(n1)CCCC2 ; *c1cnc2c(c1)NCCC2 ; *c1ccc2c(n1)CCCN2]

In embodiments, R² is

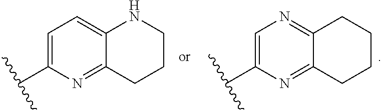 [SMILES: *c1ccc2c(n1)CCCN2 ; *c1cnc2c(n1)CCCC2]

In embodiments of the compounds of formula (I) or (I′), or a pharmaceutically acceptable salt, a stereoisomer, or a deuterated form thereof, R² is

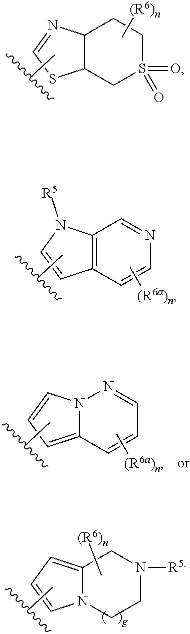

In embodiments of the compounds of formula (I), or a pharmaceutically acceptable salt, a stereoisomer, or a deuterated form thereof, R² is

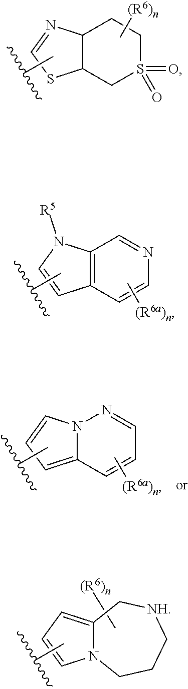

In embodiments, n is 1 or 2. In embodiments, R⁶ is each independently halogen, —OH, oxo, —CN, —C₁-C₆ alkyl, or —CONH₂ and R^{6a} is each independently halogen, —OH, —CN, —C₁-C₆ alkyl, or —CONH₂. In embodiments, R⁶ is each independently —OH or oxo. In embodiments, R^{6a} is each independently —OH. In embodiments, R² is

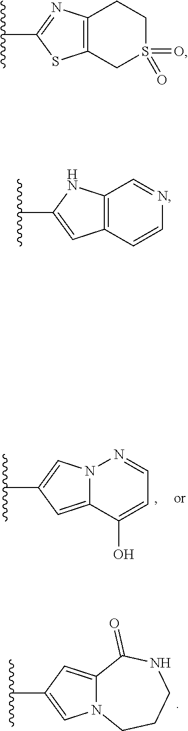 [SMILES: *c1nc2c(s1)CS(=O)(=O)CC2 ; *c1cc2ccncc2[nH]1 ; *c1cc2c(O)ccnn2c1 ; *c1cc2n(c1)CCCNC2=O]

In embodiments of the compounds of formula (I) or (I′), or a pharmaceutically acceptable salt, a stereoisomer, or a deuterated form thereof, R² is

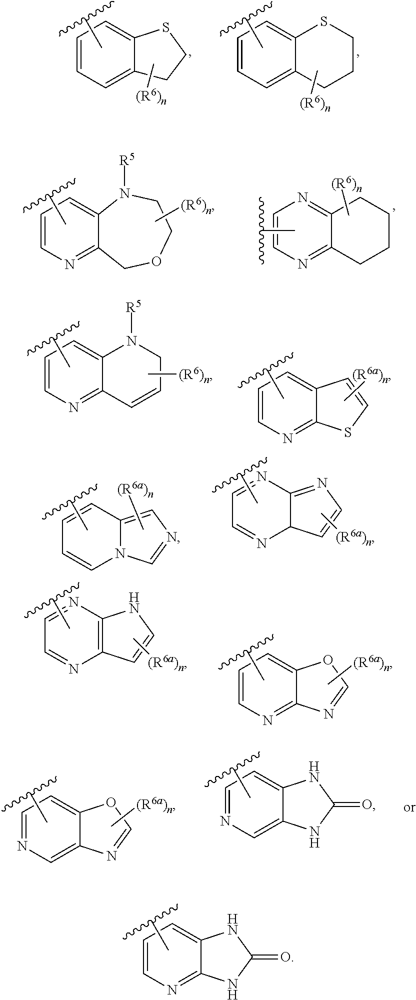

In embodiments, n is 1 or 2. In embodiments, R⁶ is each independently halogen, —OH, oxo, —CN, —C₁-C₆ alkyl, or —CONH₂ and R^{6a} is each independently halogen, —OH, —CN, —C₁-C₆ alkyl, or —CONH₂. In embodiments, R⁶ is each independently —OH, oxo, -methyl, or —CONH₂. In embodiments, R^{6a} is each independently —OH, -methyl, or —CONH₂. In embodiments, R² is

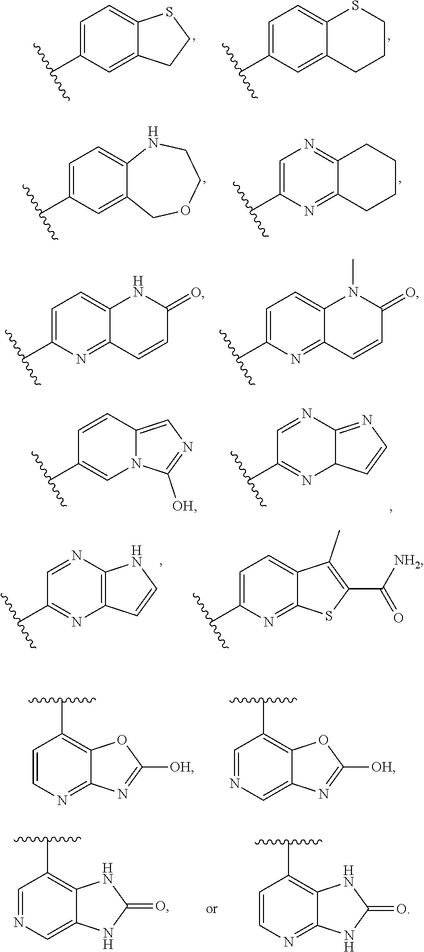

In embodiments, R² is

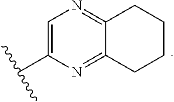 [SMILES: *c1cnc2c(n1)CCCC2]

In embodiments of the compounds of formula (I) or (I′), or a pharmaceutically acceptable salt, a stereoisomer, or a deuterated form thereof, R² is

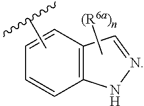

In embodiments, n is 1 or 2. In embodiments, R^{6a} is each independently halogen, —OH, —CN, —C₁-C₆ alkyl, or —CONH₂. In embodiments, R^{6a} is each independently halogen, or methyl. In embodiments, R² is

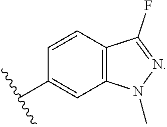 [SMILES: *c1ccc2c(F)nn(C)c2c1]

In embodiments of the compounds of formula (I) or (I′), or a pharmaceutically acceptable salt, a stereoisomer, or a deuterated form thereof, each R^{6a} is independently halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂N(C₁-C₃ alkyl), —SO₂NR⁷R⁸, or 4-6 membered heterocycle. In embodiments, R^{6a} is independently halogen, —OH, —CN, —SO₂CH₃,

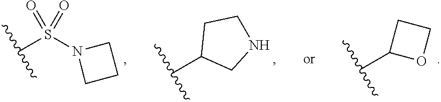 [SMILES: *S(=O)(=O)N1CCC1 ; *C1CCNC1 ; *C1CCO1]

In embodiments of the compounds of formula (I) or (I′), or a pharmaceutically acceptable salt, a stereoisomer, or a deuterated form thereof, the compound has the structure of formula (I-A):

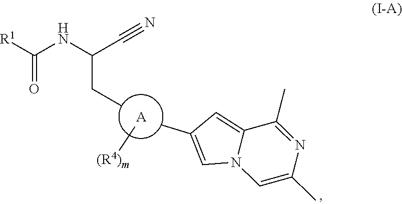

or a pharmaceutically acceptable salt, a stereoisomer, or a deuterated form thereof, wherein:
ring A is a phenyl or heteroaryl ring;
R¹ is

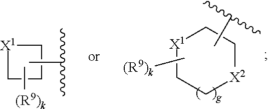

each R⁴ is independently halogen, —C₁-C₆ alkyl, —C₁-C₆ haloalkyl, or —CN;
each R⁹ is independently halogen, —OH, —C₁-C₆ alkyl, —C₁-C₆ alkyl-OH, or —OC₁-C₆ alkyl;
X¹ is —O—, —S—, or —NH—;
X² is —O—, —S—, —NH—, or —CR¹¹R¹²—;
R¹¹ and R¹² are each independently hydrogen, halogen, or —C₁-C₆ alkyl;
g is 1, 2, or 3;
k is 0, 1, or 2; and
m is 0, 1, or 2.

In embodiments of the compounds of formula (I), (I′), or (I-A), or a pharmaceutically acceptable salt, a stereoisomer, or a deuterated form thereof, ring A is phenyl and R¹ is

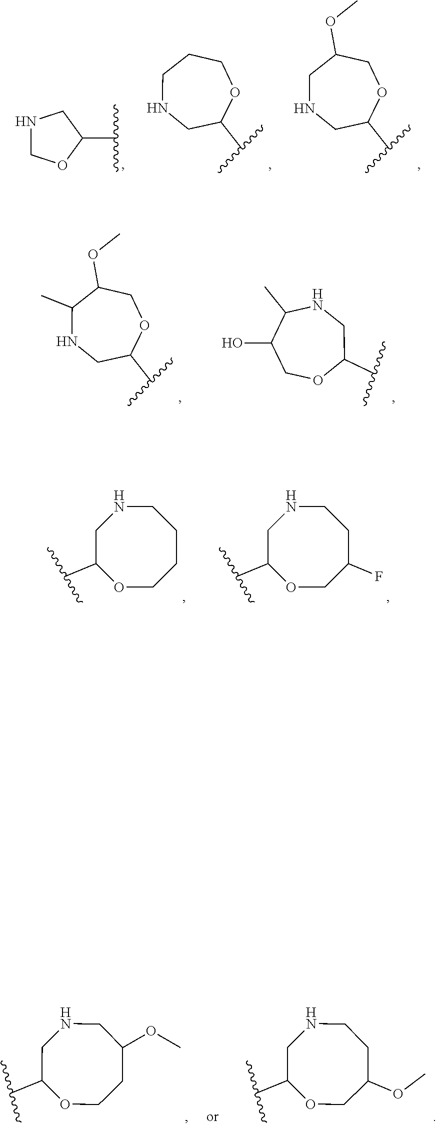

In embodiments, m is 0 or 1 and R⁴ is halogen. In embodiments, A is

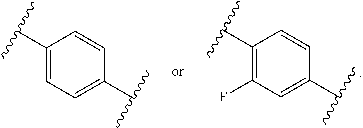 [SMILES: *c1ccc(*)cc1 ; *c1ccc(*)c(F)c1]

In embodiments of the compounds of formula (I) or (I′), or a pharmaceutically acceptable salt, a stereoisomer, or a deuterated form thereof, the compound has the structure of formula (I-B):

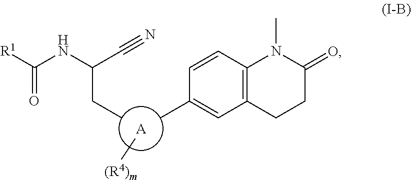

or a pharmaceutically acceptable salt, a stereoisomer, or a deuterated form thereof, wherein:
ring A is a phenyl or heteroaryl ring;
R¹ is

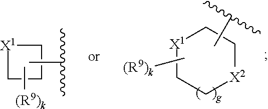

each R⁴ is independently halogen, —C₁-C₆ alkyl, —C₁-C₆ haloalkyl, or —CN;
each R⁹ is independently halogen, —OH, —C₁-C₆ alkyl, —C₁-C₆ alkyl-OH, or —OC₁-C₆ alkyl;
X¹ is —O—, —S—, or —NH—;
X² is —O—, —S—, —NH—, or —CR¹¹R¹²—;
R¹¹ and R¹² are each independently hydrogen, halogen, or —C₁-C₆ alkyl;
g is 1, 2, or 3;
k is 0, 1, or 2; and
m is 0, 1, or 2.

In embodiments of the compounds of formula (I) or (I′), or a pharmaceutically acceptable salt, a stereoisomer, or a deuterated form thereof, the compound has the structure of formula (I-C):

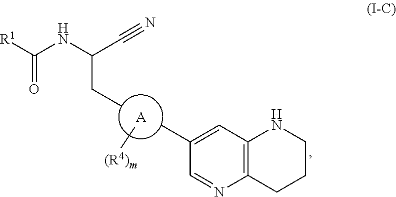

or a pharmaceutically acceptable salt, a stereoisomer, or a deuterated form thereof, wherein:
ring A is a phenyl or heteroaryl ring;
R¹ is

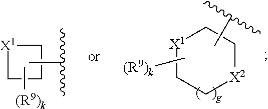

each R⁴ is independently halogen, —C₁-C₆ alkyl, —C₁-C₆ haloalkyl, or —CN;
each R⁹ is independently halogen, —OH, —C₁-C₆ alkyl, —C₁-C₆ alkyl-OH, or —OC₁-C₆ alkyl;
X¹ is —O—, —S—, or —NH—;
X² is —O—, —S—, —NH—, or —CR¹¹R¹²—;
R¹¹ and R¹² are each independently hydrogen, halogen, or —C₁-C₆ alkyl;
g is 1, 2, or 3;
k is 0, 1, or 2; and
m is 0, 1, or 2.

In embodiments of the compounds of formula (I-B) or (I-C), or a pharmaceutically acceptable salt, a stereoisomer, or a deuterated form thereof, ring A is phenyl and R¹ is

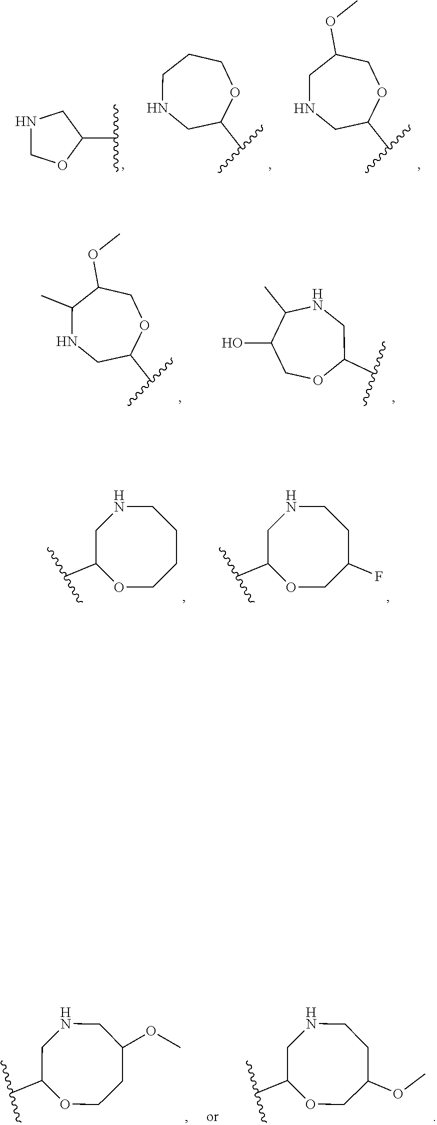

In embodiments, m is 0 or 1 and R⁴ is halogen. In embodiments, A is

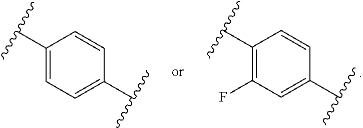 [SMILES: *c1ccc(*)cc1 ; *c1ccc(*)c(F)c1]

In embodiments of the compounds of formula (I) or (I′), or a pharmaceutically acceptable salt, a stereoisomer, or a deuterated form thereof, the compound has the structure of formula (I-D):

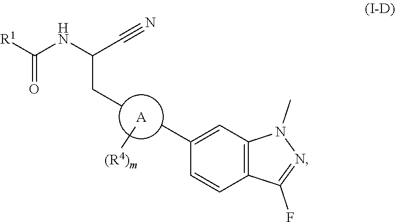

or a pharmaceutically acceptable salt, a stereoisomer, or a deuterated form thereof, wherein:
ring A is a heteroaryl ring;
R¹ is

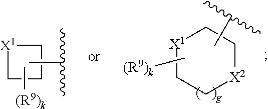

each R⁴ is independently halogen, —C₁-C₆ alkyl, —C₁-C₆ haloalkyl, or —CN;
each R⁹ is independently halogen, —OH, —C₁-C₆ alkyl, —C₁-C₆ alkyl-OH, or —OC₁-C₆ alkyl;
X¹ is —O—, —S—, or —NH—;
X² is —O—, —S—, —NH—, or —CR¹¹R¹²—;
R¹¹ and R¹² are each independently hydrogen, halogen, or —C₁-C₆ alkyl;
g is 1, 2, or 3;
k is 0, 1, or 2; and
m is 0, 1, or 2.

In embodiments of the compounds of formula (I-C), or a pharmaceutically acceptable salt, a stereoisomer, or a deuterated form thereof, ring A is phenyl or thiazole and R¹ is

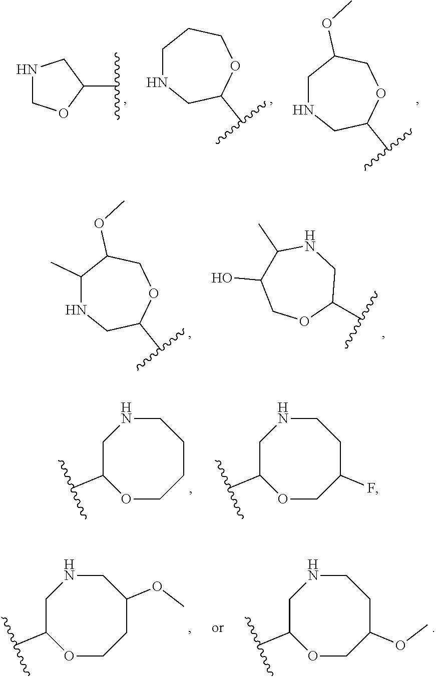

In embodiments, m is 0 or 1 and R⁴ is halogen. In embodiments, A is

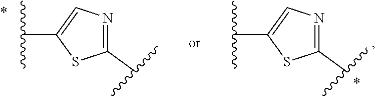 [SMILES: *c1cnc(*)s1 ; *c1cnc(*)s1]

wherein * denotes connectivity to the indazole ring. In embodiments, A is

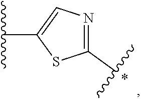 [SMILES: *c1cnc(*)s1]

wherein * denotes connectivity to the indazole ring.

In embodiments of the compounds of formula (I), (I′), (I-A), (I-B), (I-C), or (I-D), or a pharmaceutically acceptable salt, a stereoisomer, or a deuterated form thereof, wherein:
R¹ is

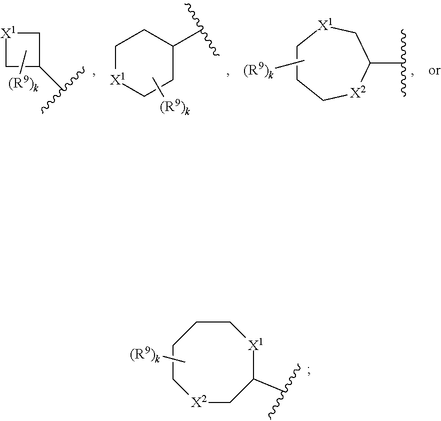

X¹ is —O—, —S—, or —NH—;
X² is —O—, —S—, —NH—, or —CH₂—; and
each R⁹ is independently —OH, fluoro, methyl, ethyl, —CH₂OH, or —OCH₃.

In embodiments of the compounds of formula (I) or (I′), or a pharmaceutically acceptable salt, a stereoisomer, or a deuterated form thereof, the compound has the structure of formula (I-E):

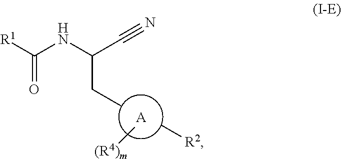

or a pharmaceutically acceptable salt, a stereoisomer, or a deuterated form thereof, wherein:
ring A is a phenyl or heteroaryl ring;
R¹ is

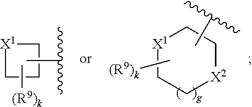

R² is

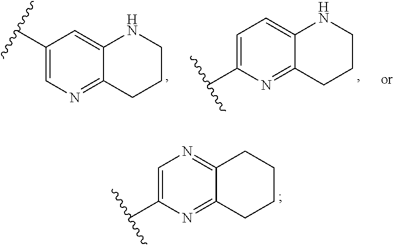 [SMILES: *c1cnc2c(c1)NCCC2 ; *c1ccc2c(n1)CCCN2 ; *c1cnc2c(n1)CCCC2]

each R⁴ is independently halogen, —C₁-C₆ alkyl, —C₁-C₆ haloalkyl, or —CN;
each R⁹ is independently halogen, —OH, —C₁-C₆ alkyl, —C₁-C₆ alkyl-OH, or —OC₁-C₆ alkyl;
X¹ is —O—, —S—, or —NH—;
X² is —O—, —S—, —NH—, or —CR¹¹R¹²—;
R¹¹ and R¹² are each independently hydrogen, halogen, or —C₁-C₆ alkyl;
g is 1, 2, or 3;
k is 0, 1, or 2; and
m is 0, 1, or 2.

In embodiments of the compounds of formula (I-E), or a pharmaceutically acceptable salt, a stereoisomer, or a deuterated form thereof, ring A is phenyl and R¹ is

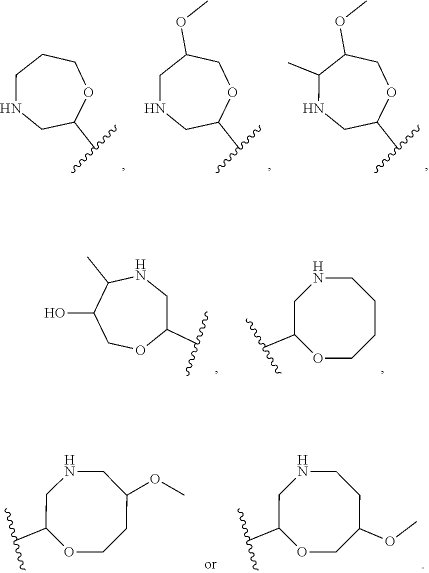

In embodiments, m is 0 or 1 and R⁴ is halogen. In embodiments, A is

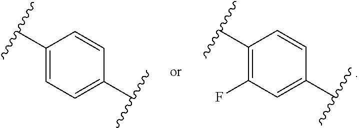 [SMILES: *c1ccc(*)cc1 ; *c1ccc(*)c(F)c1]

In embodiments of the compounds of formula (I), (I′), (I-A), (I-B), (I-C), (I-D), or (I-E), or a pharmaceutically acceptable salt, a stereoisomer, or a deuterated form thereof, R¹ is

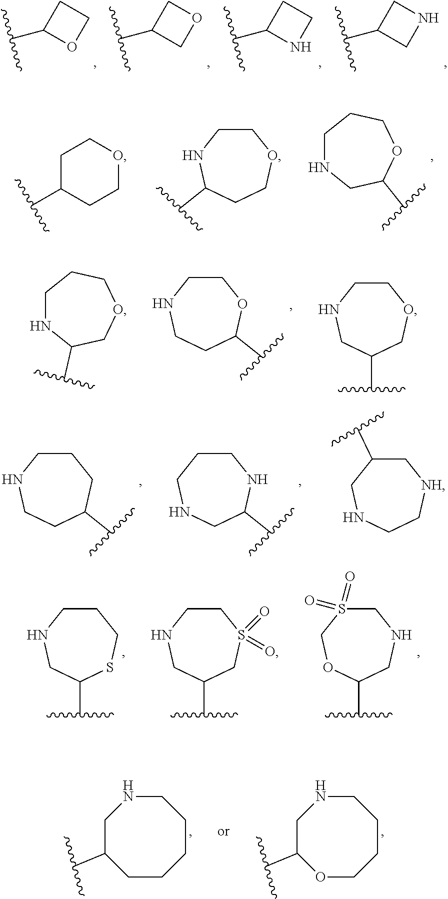

each of which is optionally substituted with 1, 2, or 3 R⁹. In embodiments, R¹ is

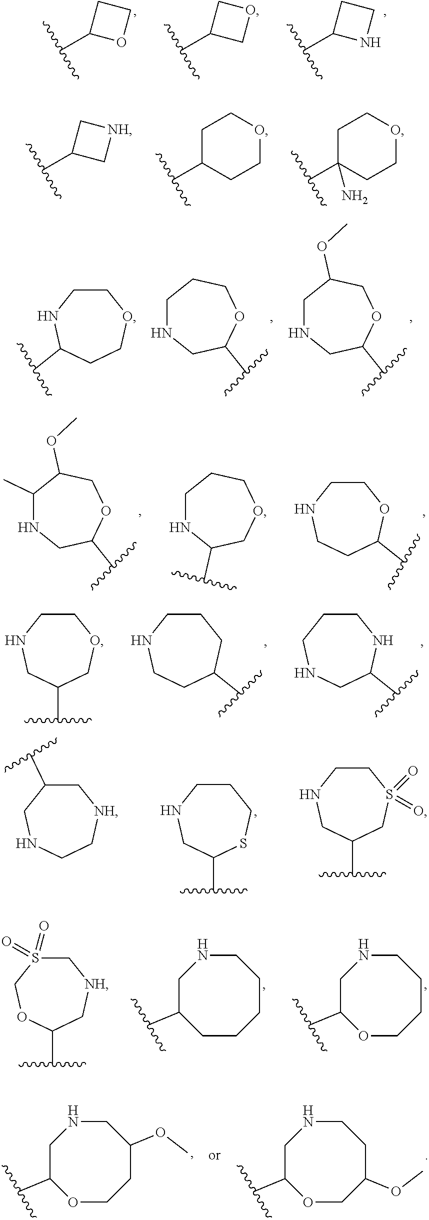

In embodiments, R¹ is

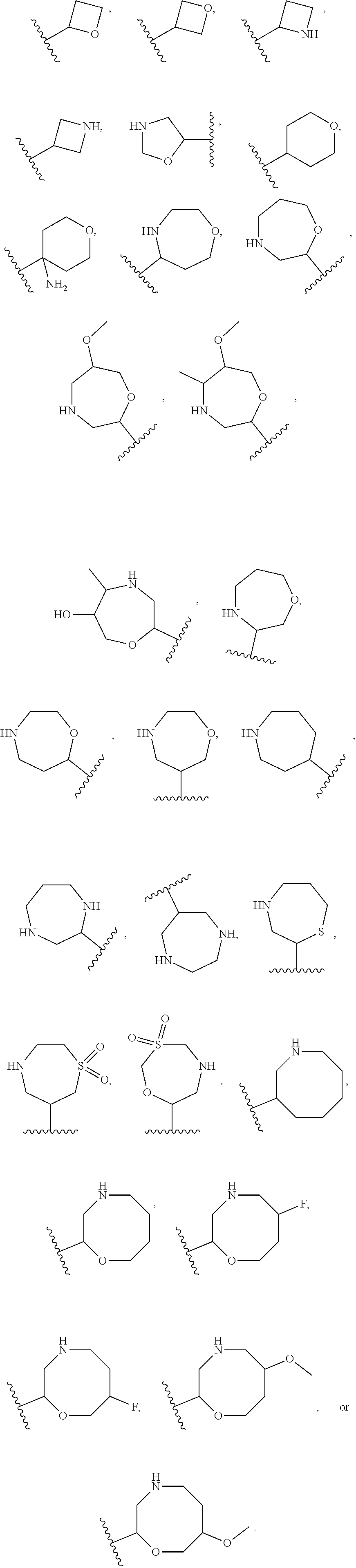

In embodiments, R¹ is

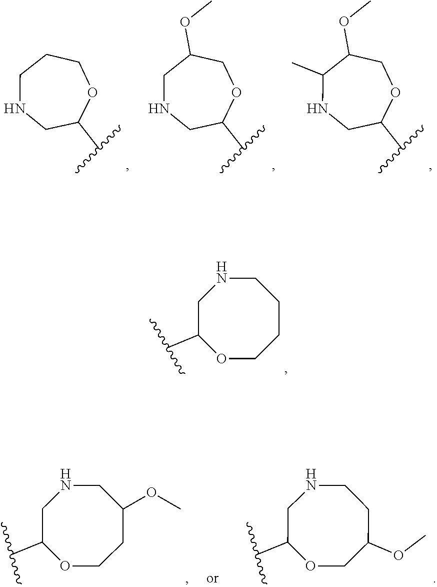

In embodiments, R¹ is

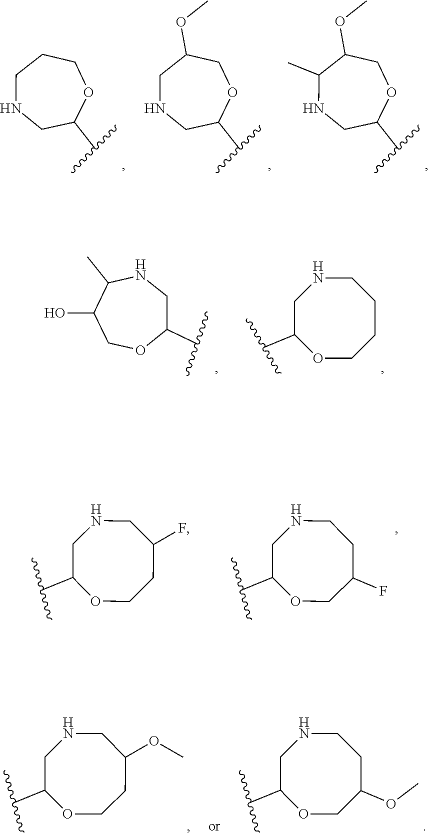

In embodiments, R¹ is

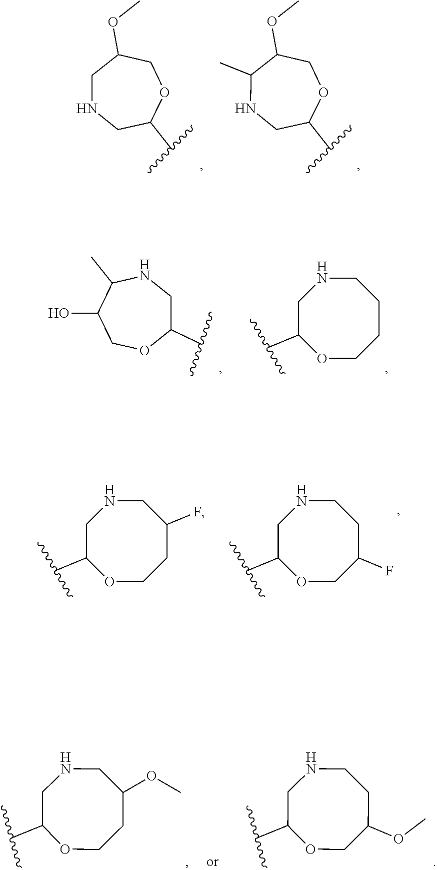

In embodiments of the compounds of formula (I), (I′), (I-A), (I-B), (I-C), (I-D), or (I-E), or a pharmaceutically acceptable salt, a stereoisomer, or a deuterated form thereof, A is aryl or heteroaryl. In embodiments, A is a monocyclic or a bicyclic ring.

In embodiments of the compounds of formula (I), (I′), (I-A), (I-B), (I-C), (I-D), or (I-E), or a pharmaceutically acceptable salt, a stereoisomer, or a deuterated form thereof, A is a phenyl ring. In embodiments, A is

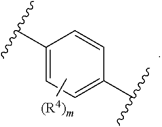

In embodiments, m is 1 or 2 and R⁴ is each independently a halogen. In embodiments, A is

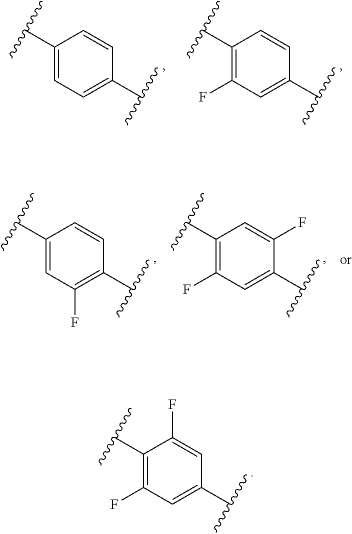 [SMILES: *c1ccc(*)cc1 ; *c1ccc(*)c(F)c1 ; *c1ccc(*)c(F)c1 ; *c1cc(F)c(*)cc1F ; *c1cc(F)c(*)c(F)c1]

In embodiments of the compounds of formula (I), (I′), (I-A), (I-B), (I-C), (I-D), or (I-E), or a pharmaceutically acceptable salt, a stereoisomer, or a deuterated form thereof, A is

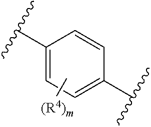

### and R¹ is

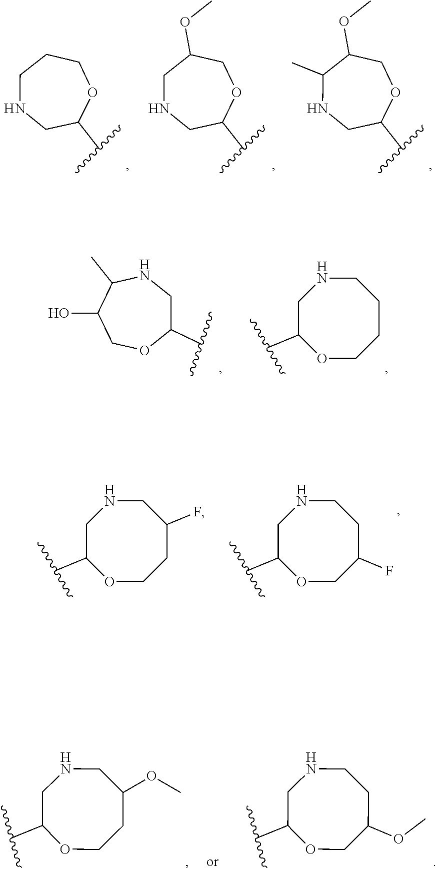

In embodiments, A is

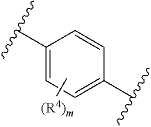

### and R¹ is

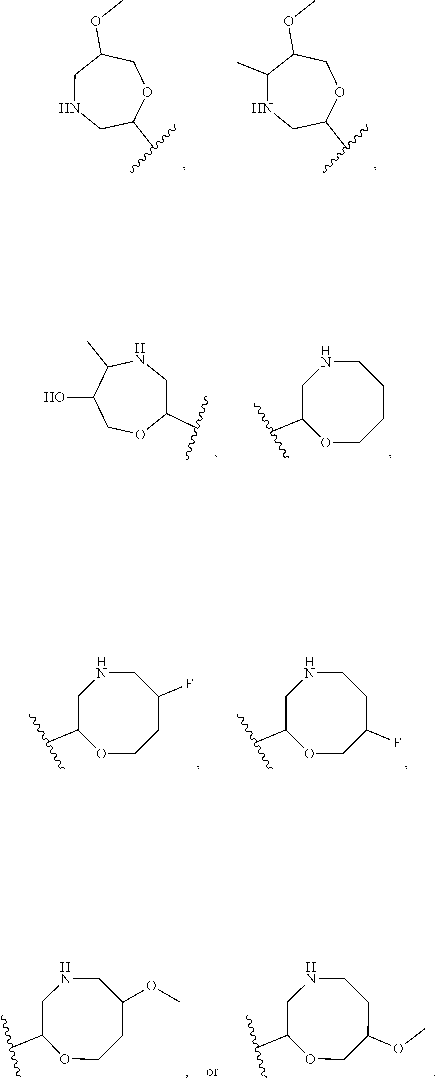

In embodiments of the compounds of formula (I), (I′), (I-A), (I-B), (I-C), (I-D), or (I-E), or a pharmaceutically acceptable salt, a stereoisomer, or a deuterated form thereof, A is

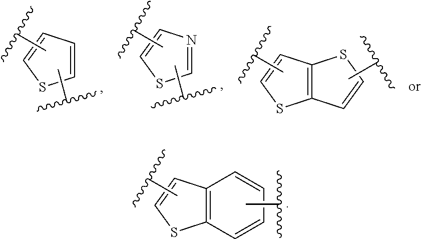

In embodiments, A is

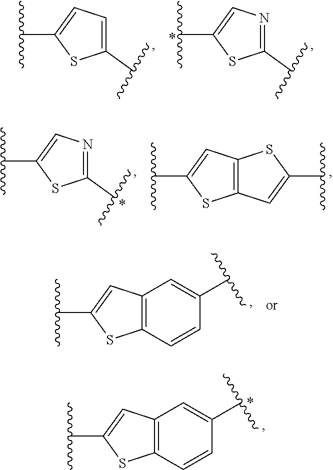

wherein * denotes connectivity to R². In embodiments, A is

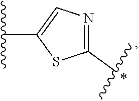 [SMILES: *c1cnc(*)s1]

wherein * denotes connectivity to R².

In embodiments of the compounds of formula (I), or a pharmaceutically acceptable salt, a stereoisomer, or a deuterated form thereof, R¹ is

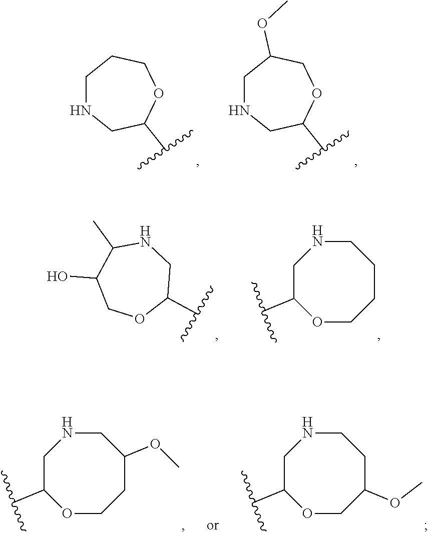

### R² is

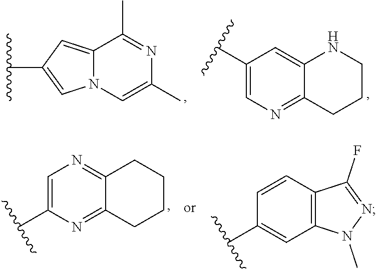

### and A is

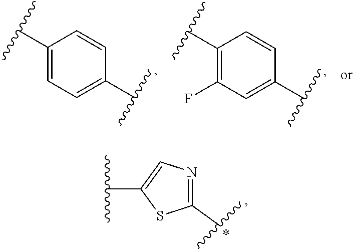 [SMILES: *c1ccc(*)cc1 ; *c1ccc(*)c(F)c1 ; *c1cnc(*)s1]

wherein * denotes connectivity to R².

In embodiments of the compounds of formula (I-A), or a pharmaceutically acceptable salt, a stereoisomer, or a deuterated form thereof, R¹ is

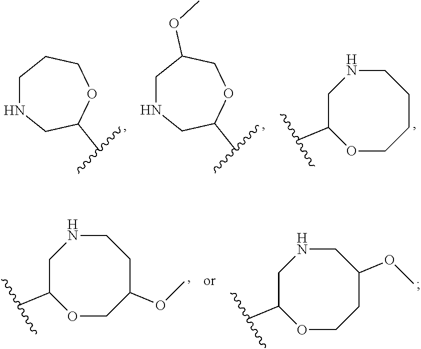

### and A is

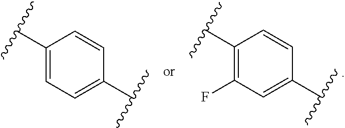 [SMILES: *c1ccc(*)cc1 ; *c1ccc(*)c(F)c1]

In embodiments of the compounds of formula (I-E), or a pharmaceutically acceptable salt, a stereoisomer, or a deuterated form thereof, R¹ is

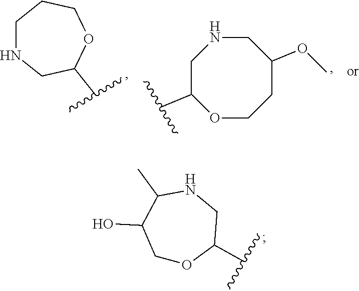

### R² is

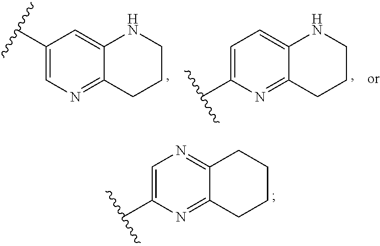

### and A is

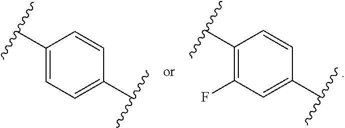 [SMILES: *c1ccc(*)cc1 ; *c1ccc(*)c(F)c1]

Embodiments provided herein for formula (I) or (I′) can be applied for formula (I-A), (I-B), (I-C), (I-D), and/or (I-E), if appropriate under the definitions of formula (I-A), (I-B), (I-C), (I-D), and/or (I-E).

In embodiments, the present disclosure provides a compound of formula (II):

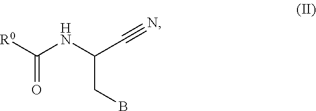

or a pharmaceutically acceptable salt, a stereoisomer, or a deuterated form thereof, wherein:
B is

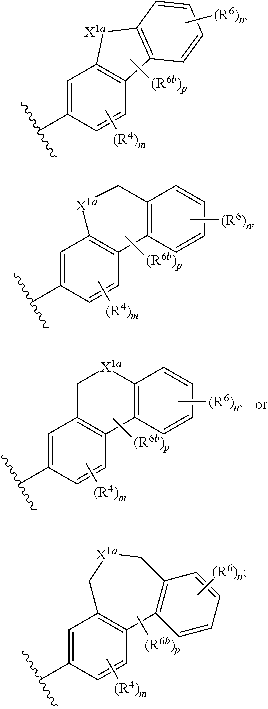

X^{1a} is —NR⁵—, —O—, —CR¹¹R¹²—, —C(O)—, —S—, —S(O)— or —S(O)₂—;
R¹ is

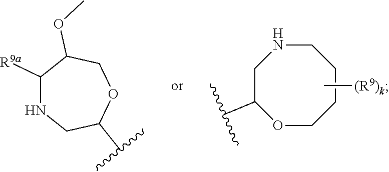

each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is each independently, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R^{6b} is each independently, halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
R^{9a} is C₁-C₃ alkyl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
k is 0, 1, 2, 3, or 4;
m is 0, 1, 2, or 3;
n is 0, 1, 2, or 3; and
p is 0, 1, or 2.

In embodiments of the compounds of formula (II), or a pharmaceutically acceptable salt, a stereoisomer, or a deuterated form thereof, X^{1a} is —NH—, —NCH₃—, —O—, —CH₂—, or —S—. In embodiments, X^{1a} is —NH—, —NCH₃—, —O—, or —CH₂—. In embodiments, X^{1a} is —NH— or —O—.

In embodiments of the compounds of formula (II), or a pharmaceutically acceptable salt, a stereoisomer, or a deuterated form thereof, each R⁴ is independently halogen, —C₁-C₃ alkyl, or —CN.

In embodiments of the compounds of formula (II), or a pharmaceutically acceptable salt, a stereoisomer, or a deuterated form thereof, each R⁶ is independently halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂N(C₁-C₃ alkyl), —SO₂NR⁷R⁸, —(C₁-C₃ alkylene)-(4-6 membered heterocycle), or 4-6 membered heterocycle, wherein each heterocycle is optionally substituted by 1 or 2 R¹⁰. In some embodiments, R⁶ is independently halogen, —OH, —CN, —SO₂CH₃,

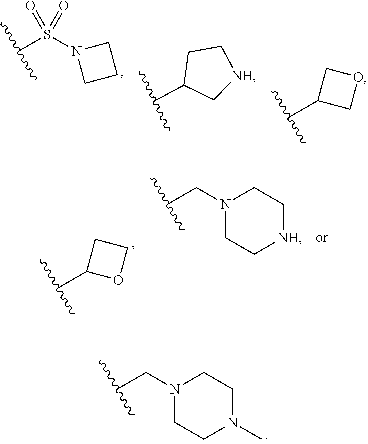

In embodiments of the compounds of formula (II), or a pharmaceutically acceptable salt, a stereoisomer, or a deuterated form thereof, each R⁶ is independently halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂N(C₁-C₃ alkyl), —SO₂NR⁷R⁸, or 4-6 membered heterocycle. In some embodiments, R⁶ is independently halogen, —OH, —CN, —SO₂CH₃,

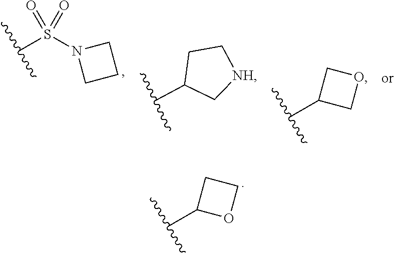

In embodiments of the compounds of formula (II), or a pharmaceutically acceptable salt, a stereoisomer, or a deuterated form thereof, m is 0 or 1.

In embodiments of the compounds of formula (II), or a pharmaceutically acceptable salt, a stereoisomer, or a deuterated form thereof, n is 0, 1, or 2.

In embodiments of the compounds of formula (II), or a pharmaceutically acceptable salt, a stereoisomer, or a deuterated form thereof, p is 0 or 1.

In embodiments of the compounds of formula (II), or a pharmaceutically acceptable salt, a stereoisomer, or a deuterated form thereof, B is

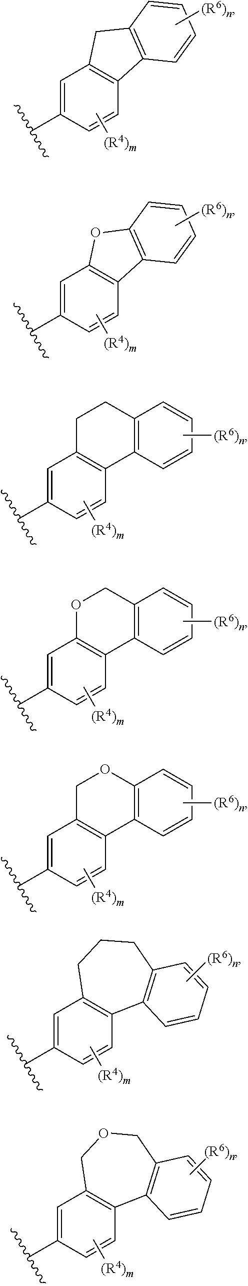

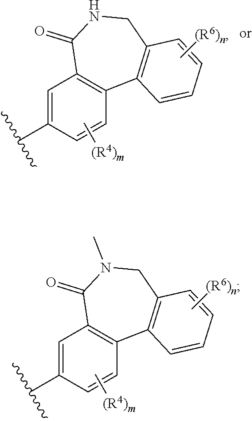

wherein m is 0 or 1 and n is 0, 1, or 2. In embodiments, R⁴ is halogen. In embodiments, R⁴ is F. In embodiments, R⁶ is halogen, —CN, or heterocycle wherein the heterocycle is optionally substituted by 1 or 2 R¹⁰. In embodiments, R⁶ is halogen, —CN, oxetane, or pyrrolidine. In embodiments, R⁶ is F, —CN,

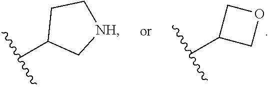 [SMILES: *C1CCNC1 ; *C1COC1]

In embodiments of the compounds of formula (II), or a pharmaceutically acceptable salt, a stereoisomer, or a deuterated form thereof, B is

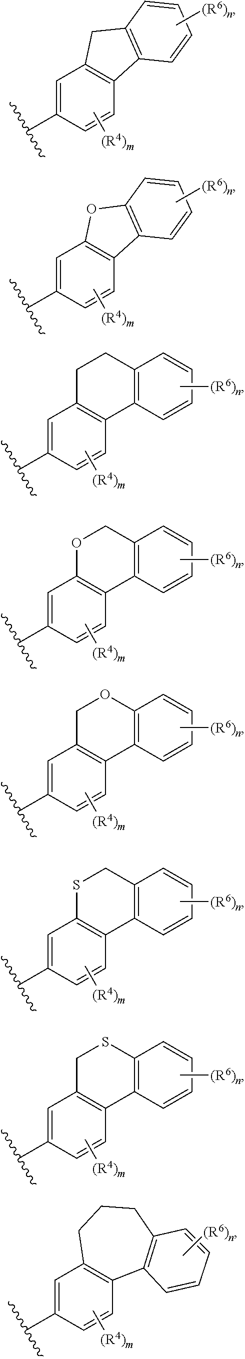

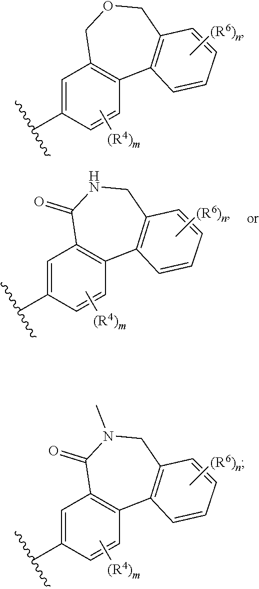

wherein m is 0 or 1 and n is 0, 1, or 2. In embodiments, R⁴ is halogen. In embodiments, R⁴ is F. In embodiments, R⁶ is halogen, —CN, or heterocycle wherein the heterocycle is optionally substituted by 1 or 2 R¹⁰. In embodiments, R⁶ is halogen, —CN, oxetane, or pyrrolidine. In embodiments, R⁶ is F, —CN,

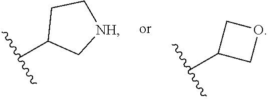 [SMILES: *C1CCNC1 ; "or" ; *C1COC1]

In embodiments of the compounds of formula (II), or a pharmaceutically acceptable salt, a stereoisomer, or a deuterated form thereof, B is

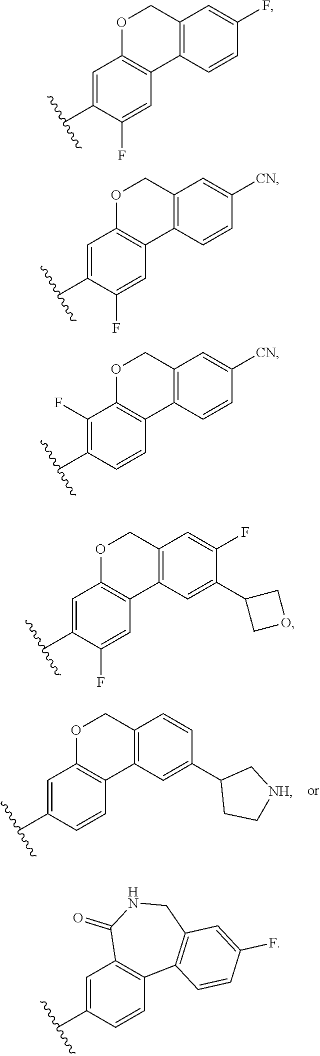

In embodiments of the compounds of formula (II), or a pharmaceutically acceptable salt, a stereoisomer, or a deuterated form thereof. B is

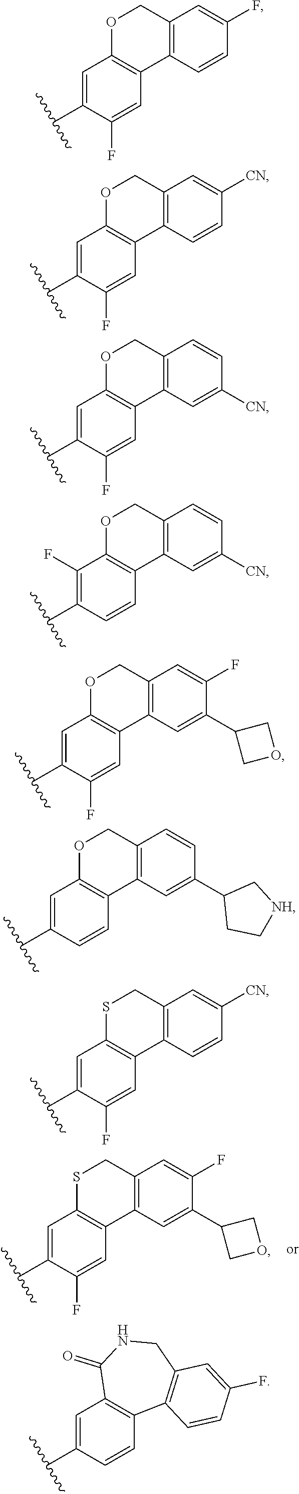

In embodiments of the compounds of formula (II), or a pharmaceutically acceptable salt, a stereoisomer, or a deuterated form thereof, B is

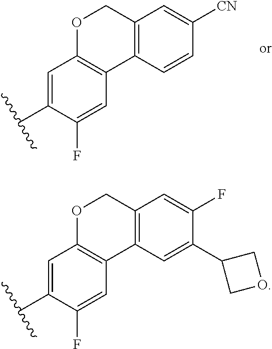 [SMILES: *c1cc2c(cc1F)-c1ccc(C#N)cc1CO2 ; "or" ; *c1cc2c(cc1F)-c1cc(C3COC3)c(F)cc1CO2]

In embodiments of the compounds of formula (II), or a pharmaceutically acceptable salt, a stereoisomer, or a deuterated form thereof, B is

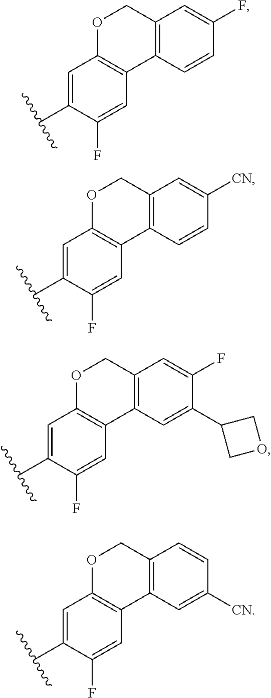

In embodiments of the compounds of formula (II), or a pharmaceutically acceptable salt, a stereoisomer, or a deuterated form thereof, the compound has the structure of formula (II-A):

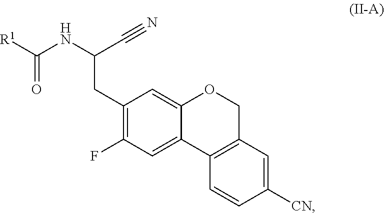

or a pharmaceutically acceptable salt, a stereoisomer, or a deuterated form thereof wherein:
R¹ is

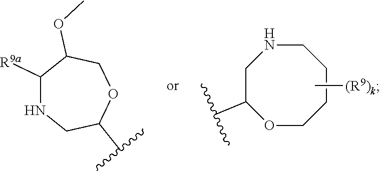

each R⁹ is independently halogen, —OH, —C₁-C₆ alkyl, —C₁-C₆ alkyl-OH, or —OC₁-C₆ alkyl;
R^{9a} is C₁-C₃ alkyl; and
k is 0, 1, or 2.

In embodiments of the compounds of formula (II), or a pharmaceutically acceptable salt, a stereoisomer, or a deuterated form thereof, the compound has the structure of formula (II-B):

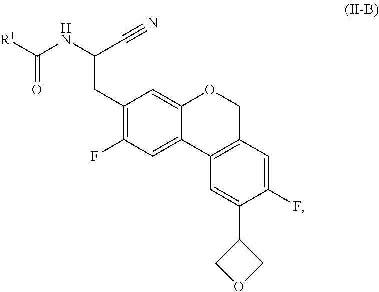

or a pharmaceutically acceptable salt, a stereoisomer, or a deuterated form thereof wherein:
R¹ is

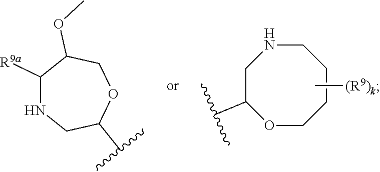

each R⁹ is independently halogen, —OH, —C₁-C₆ alkyl, —C₁-C₆ alkyl-OH, or —OC₁-C₆ alkyl;
R^{9a} is C₁-C₃ alkyl; and
k is 0, 1, or 2.

In embodiments of the compounds of formula (II), (II-A), or (II-B), or a pharmaceutically acceptable salt, a stereoisomer, or a deuterated form thereof, k is 0, 1 or 2. In embodiments, k is 0 or 1.

In embodiments of the compounds of formula (II), (II-A), or (II-B), or a pharmaceutically acceptable salt, a stereoisomer, or a deuterated form thereof, R⁹ is each independently —OH, —OCH₃, or —CH₃.

In embodiments of the compounds of formula (II), (II-A), or (II-B), or a pharmaceutically acceptable salt, a stereoisomer, or a deuterated form thereof, R^{9a} is —CH₃.

In embodiments of the compounds of formula (II), (II-A), or (II-B), or a pharmaceutically acceptable salt, a stereoisomer, or a deuterated form thereof, R¹ is

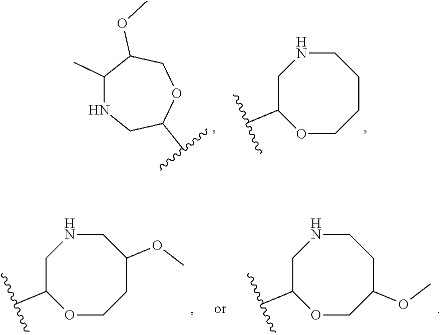 [SMILES: *C1CNC(C)C(OC)CO1 ; *C1CNCCCCO1 ; *C1CNCC(OC)CCO1 ; *C1CNCCC(OC)CO1]

In embodiments of the compounds of formula (II), (II-A), or (II-B), or a pharmaceutically acceptable salt, a stereoisomer, or a deuterated form thereof, R¹ is

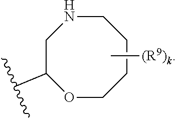

In embodiments, R⁹ is each independently —OH, —OCH₃, or —CH₃. In embodiments, R¹ is

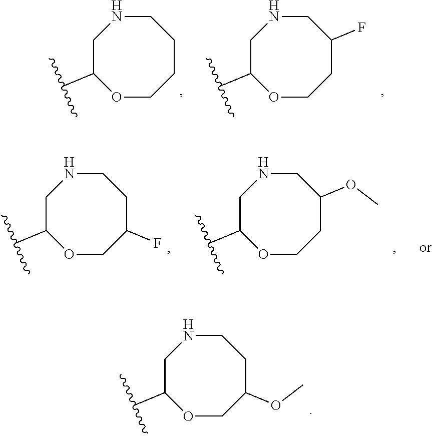 [SMILES: *C1CNCCCCO1 ; *C1CNCC(F)CCO1 ; *C1CNCCC(F)CO1 ; *C1CNCC(OC)CCO1 ; *C1CNCCC(OC)CO1]

In embodiments of the compounds of formula (II), or a pharmaceutically acceptable salt, a stereoisomer, or a deuterated form thereof, B is

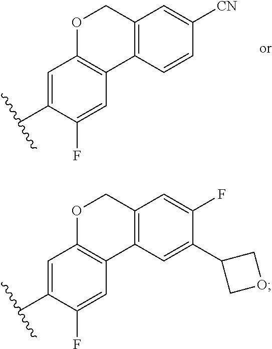 [SMILES: *c1cc2c(cc1F)-c1ccc(C#N)cc1CO2 ; "or" ; *c1cc2c(cc1F)-c1cc(C3COC3)c(F)cc1CO2]

### and R¹ is

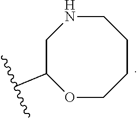 [SMILES: *C1CNCCCCO1]

Embodiments provided herein for formula (II) can be applied for formula (II-A) and/or (II-B), if appropriate under the definitions of formula (II-A) and/or (II-B).

In embodiments, the present disclosure provides a compound of formula (III):

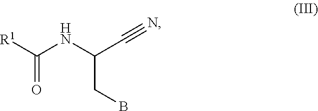

or a pharmaceutically acceptable salt, a stereoisomer, or a deuterated form thereof, wherein:
B is

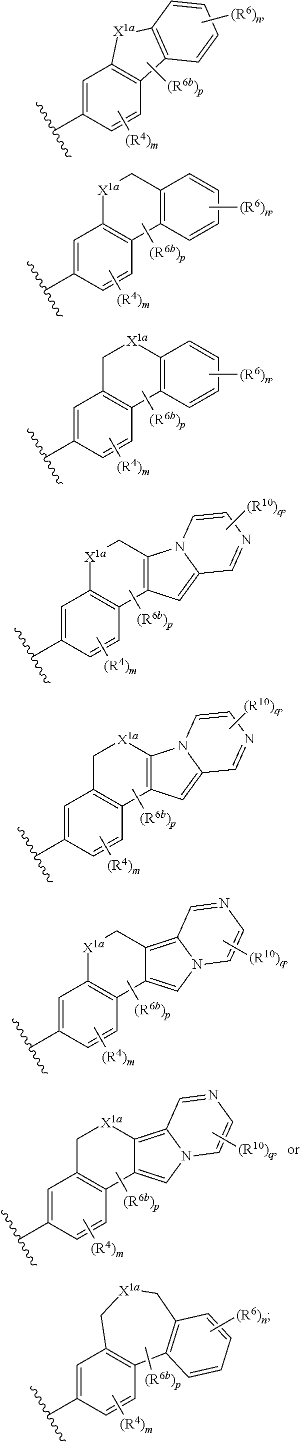

X^{1a} is —NR⁵—, —O—, —CR¹¹R¹²—, —C(O)—, —S—, —S(O)— or —S(O)₂—;
R¹ is

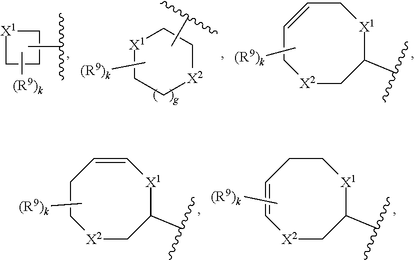

or C₁-C₆ alkylene(NH)-carbocycle substituted with 0, 1, 2, 3, or 4 R³;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
two R⁶ taken together with the atoms to which they are attached forms a monocyclic or bicyclic carbocyclyl, aryl, heteroaryl, or heterocyclyl, each optionally substituted by 1 or 2 R¹⁰;
each R^{6b} is each independently, halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
g is 0, 1, 2, or 3;
k is 0, 1, 2, 3, or 4;
m is 0, 1, 2, or 3;
n is 2; and
p is 0, 1, or 2.

In embodiments, the present disclosure provides a compound of formula (III′):

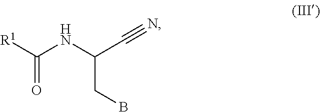

or a pharmaceutically acceptable salt, a stereoisomer, or a deuterated form thereof, wherein:
B is

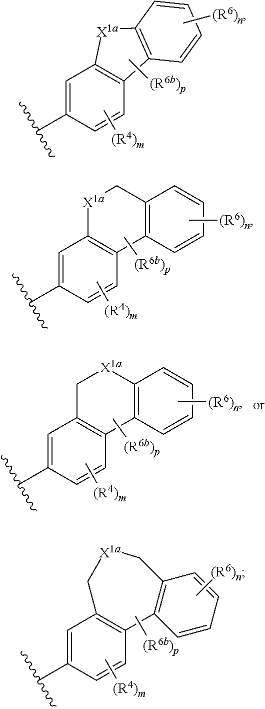

X^{1a} is —NR⁵—, —O—, —CR¹¹R¹²—, —C(O)—, —S—, —S(O)— or —S(O)₂—;
R¹ is

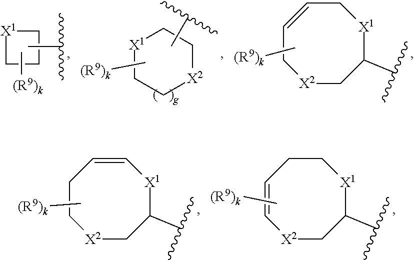

or C₁-C₆ alkylene(NH)-carbocycle substituted with 0, 1, 2, 3, or 4 R³;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
two R⁶ taken together with the atoms to which they are attached forms a monocyclic or bicyclic carbocyclyl, aryl, heteroaryl, or heterocyclyl, each optionally substituted by 1 or 2 R¹⁰;
each R^{6b} is each independently, halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
g is 0, 1, 2, or 3;
k is 0, 1, 2, 3, or 4;
m is 0, 1, 2, or 3;
n is 2; and
p is 0, 1, or 2.

In embodiments, the present disclosure provides a compound of formula (III″):

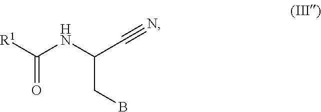

or a pharmaceutically acceptable salt, a stereoisomer, or a deuterated form thereof, wherein:
B is

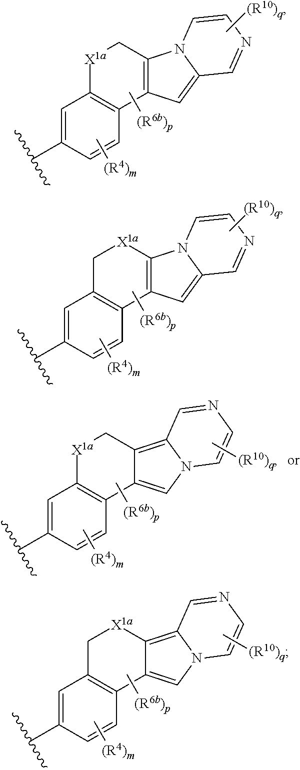

X^{1a} is —NR⁵—, —O—, —CR¹¹R¹²—, —C(O)—, —S—, —S(O)— or —S(O)₂—;
R¹ is

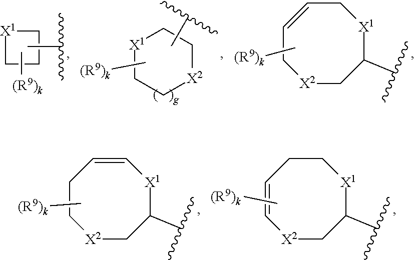

or C₁-C₆ alkylene(NH)-carbocycle substituted with 0, 1, 2, 3, or 4 R³;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R^{6b} is each independently, halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
g is 0, 1, 2, or 3;
k is 0, 1, 2, 3, or 4;
m is 0, 1, 2, or 3;
n is 2;
p is 0, 1, or 2; and
q is 0, 1, or 2.

In embodiments of the compounds of formula (III), (III′), or (III″), or a pharmaceutically acceptable salt, a stereoisomer, or a deuterated form thereof, X^{1a} is —NH—, —NCH₃—, —O—, or —CH₂—. In embodiments, X^{1a} is —O—.

In embodiments of the compounds of formula (III), (III′), or (III″), or a pharmaceutically acceptable salt, a stereoisomer, or a deuterated form thereof, two R⁶ taken together with the atoms to which they are attached forms a monocyclic 5- or 6-membered carbocyclyl, aryl, heterocyclyl, or heterocyclyl ring, each optionally substituted by 1 or 2 R¹⁰. In embodiments, two R⁶ taken together with the atoms to which they are attached forms a monocyclic 5-membered heterocyclyl ring, wherein the heterocyclyl ring optionally substituted by 1 or 2 R¹⁰. In embodiments, two R⁶ taken together with the atoms to which they are attached forms a dihydrooxazole ring, optionally substituted by 1 or 2 R¹⁰.

In embodiments of the compounds of formula (III) or (III′), or a pharmaceutically acceptable salt, a stereoisomer, or a deuterated form thereof, B is

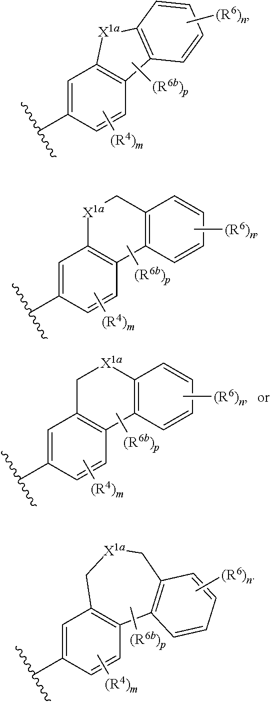

In embodiments of the compounds of formula (III), (III′), or (III″), or a pharmaceutically acceptable salt, a stereoisomer, or a deuterated form thereof, B is

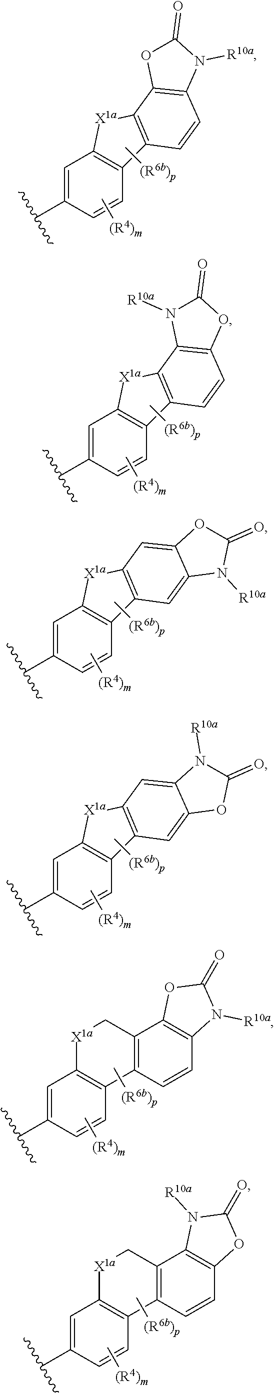

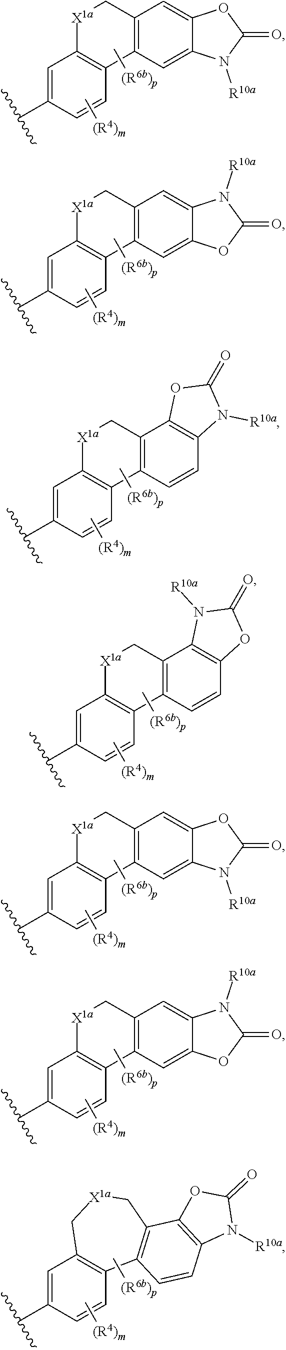

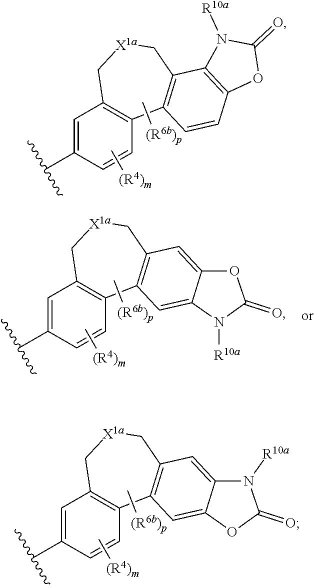

wherein each R^{10a} is independently hydrogen, —C₁-C₆ alkyl, or —C₁-C₆ alkyl-NR⁷R⁸. In embodiments, R^{10a} is hydrogen or methyl. In embodiments, X^{1a} is —O— or —S—. In embodiments, X^{1a} is —O—. In embodiments, m is 0 or 1. In embodiments, R⁴ is halogen. In embodiments, R⁴ is F. In embodiments, p is 0.

In embodiments of the compounds of formula (III) or (III′), or a pharmaceutically acceptable salt, a stereoisomer, or a deuterated form thereof, two R⁶ taken together with the atoms to which they are attached forms a bicyclic heterocyclyl ring, wherein the heterocyclyl ring optionally substituted by 1 or 2 R¹⁰. In embodiments, R⁶ taken together with the atoms to which they are attached forms a spiral bicyclic heterocyclyl ring, optionally substituted by 1 or 2 R¹⁰. In embodiments, two R⁶ taken together with the atoms to which they are attached forms an azaspirodecene ring, optionally substituted by 1 or 2 R¹⁰.

In embodiments of the compounds of formula (III) or (III′), or a pharmaceutically acceptable salt, a stereoisomer, or a deuterated form thereof, B is

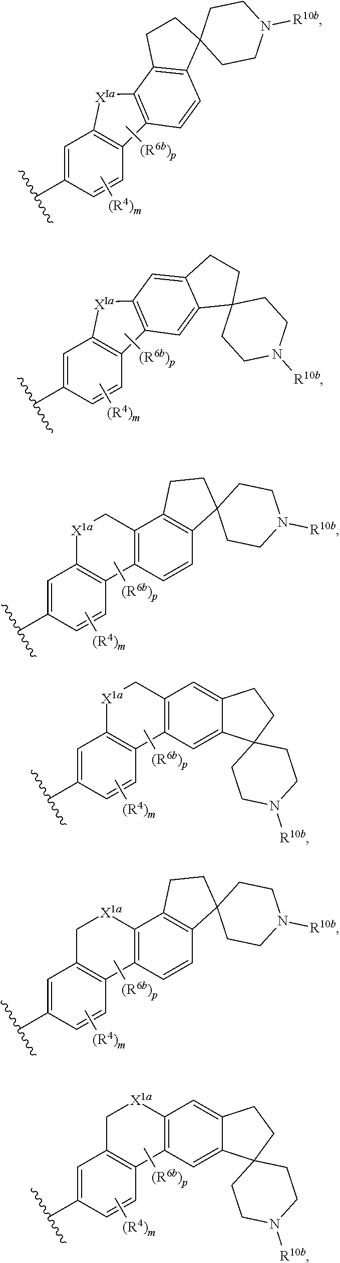

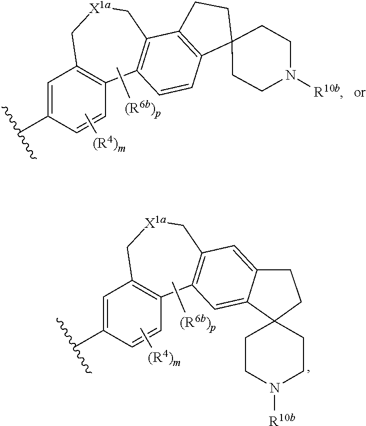

wherein each R^{10b} is independently hydrogen, —C₁-C₆ alkyl, —C₁-C₁₀ haloalkyl, —(C₁-C₆ alkylene)-carbocycle, —(C₁-C₆ alkylene)-heterocycle, carbocycle, or heterocycle. In embodiments of the compounds of formula (III) or (III′), or a pharmaceutically acceptable salt, a stereoisomer, or a deuterated form thereof, B is

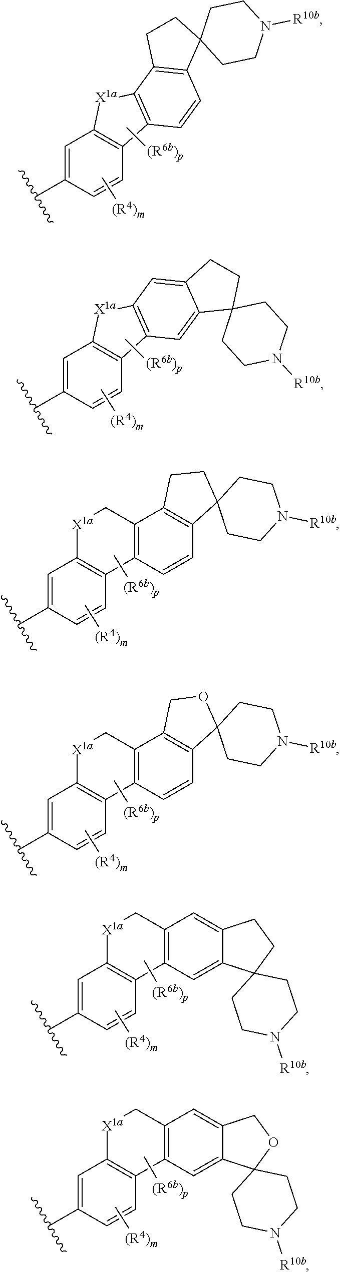

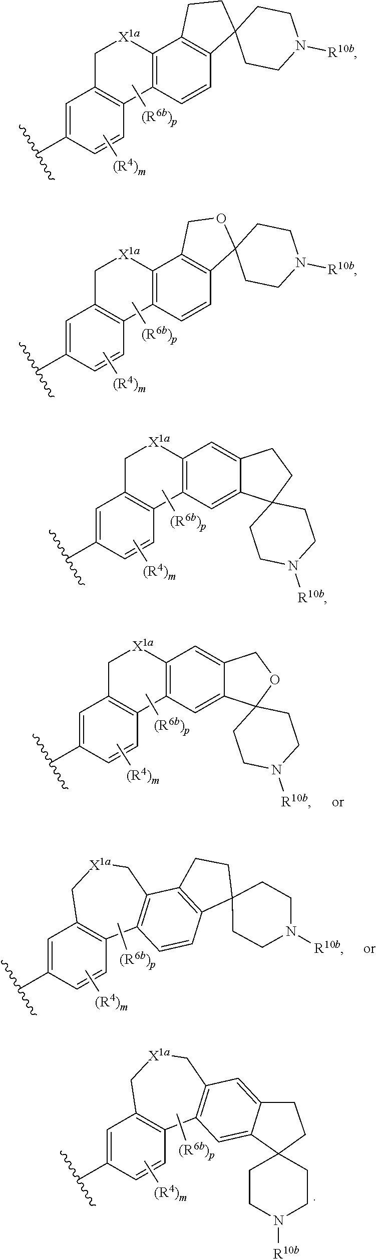

In embodiments, each R^{10b} is independently hydrogen, —C₁-C₆ alkyl, —(C₁-C₆ alkylene)-(3- to 6-membered heterocycle), or 3- to 6-membered heterocycle. In embodiments, each R^{10b} is independently hydrogen, —C₁-C₃ alkyl, or 3- to 5-membered heterocycle containing one heteroatom selected from O, S, or N. In embodiments, R^{10b} is independently hydrogen, methyl, or oxetane. In embodiments, R^{10b} is H, methyl, or

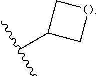 [SMILES: *C1COC1]

In embodiments, R^{10b} is hydrogen or methyl. In embodiments, X^{1a} is —O—. In embodiments, m is 0 or 1. In embodiments, R⁴ is halogen. In embodiments, R⁴ is F. In embodiments, p is 0.

In embodiments of the compounds of formula (III) or (III′), or a pharmaceutically acceptable salt, a stereoisomer, or a deuterated form thereof, B is

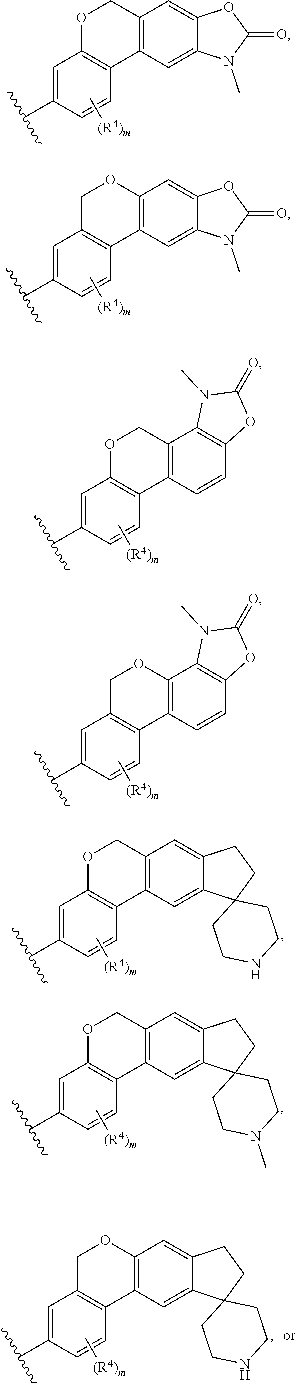

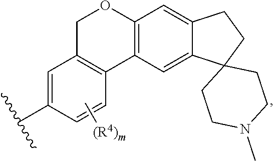

wherein m is 0 or 1. In embodiments, B is

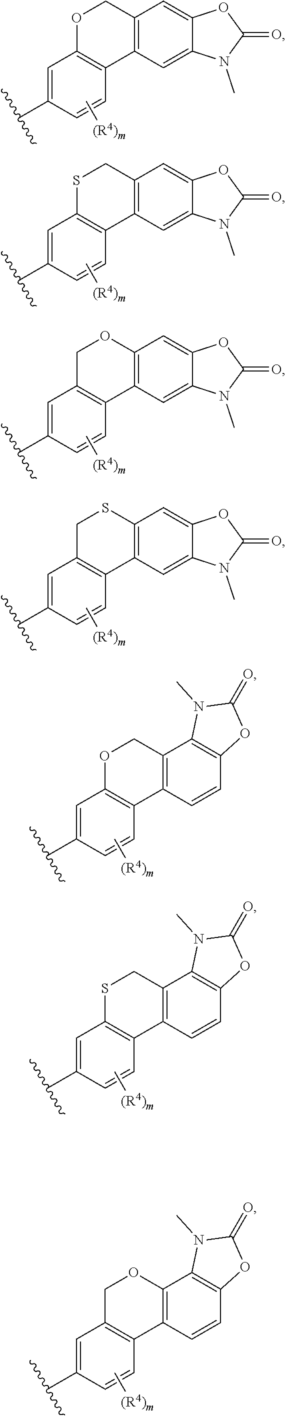

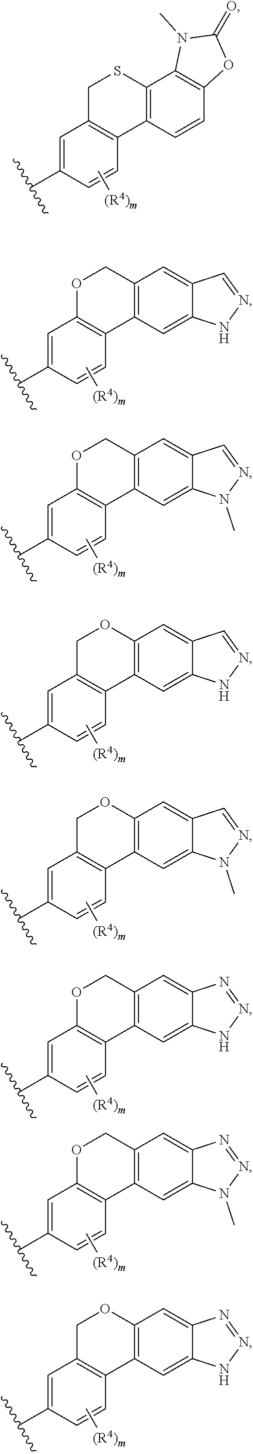

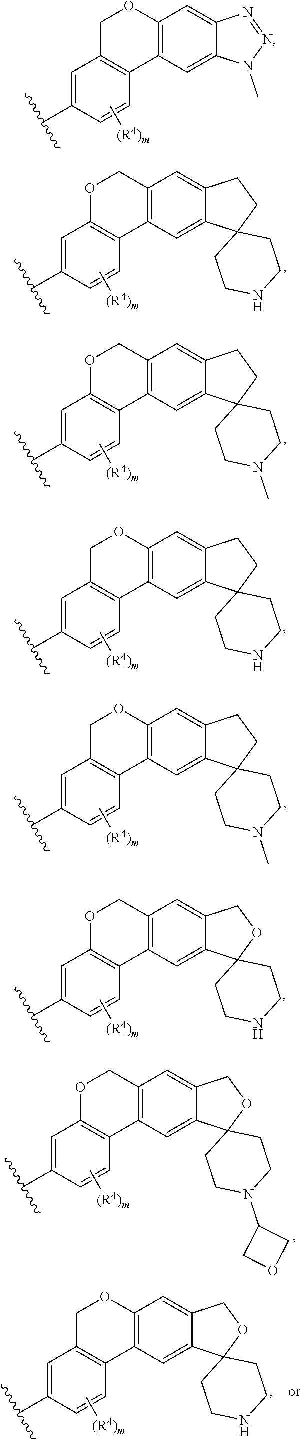

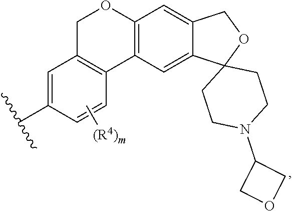

wherein m is 0 or 1. In embodiments, R⁴ is halogen.

In embodiments of the compounds of formula (III) or (III′), or a pharmaceutically acceptable salt, a stereoisomer, or a deuterated form thereof, B is

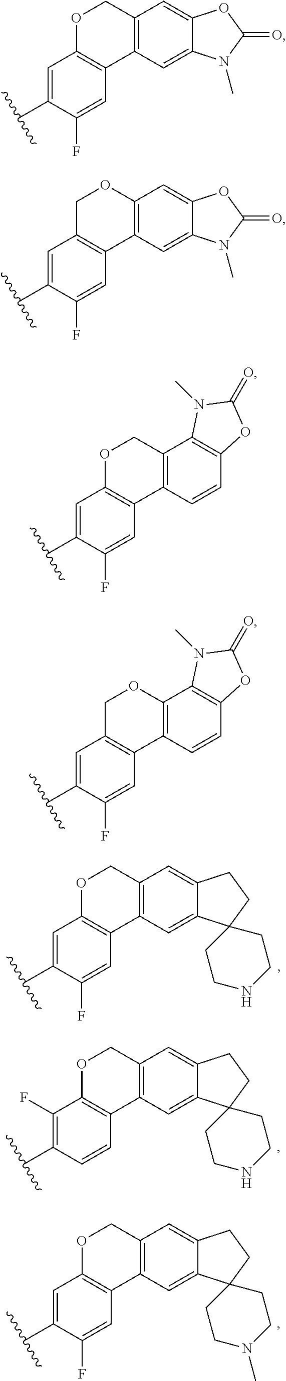

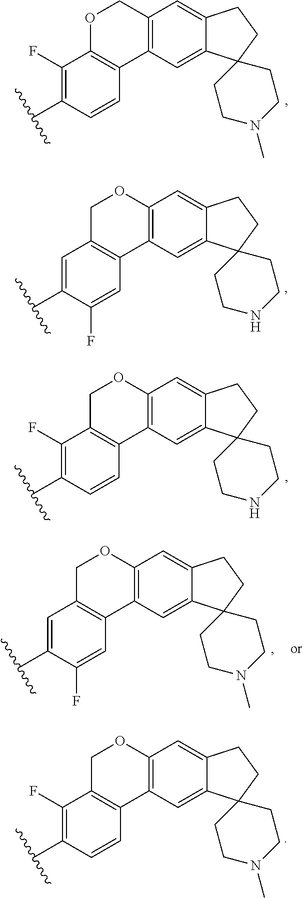

In embodiments, B is

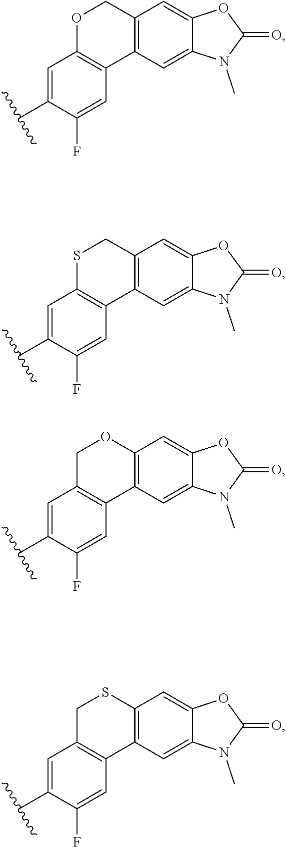 [SMILES: *c1cc2c(cc1F)-c1cc3c(cc1CO2)oc(=O)n3C ; *c1cc2c(cc1F)-c1cc3c(cc1CS2)oc(=O)n3C ; *c1cc2c(cc1F)-c1cc3c(cc1OC2)oc(=O)n3C ; *c1cc2c(cc1F)-c1cc3c(cc1SC2)oc(=O)n3C]

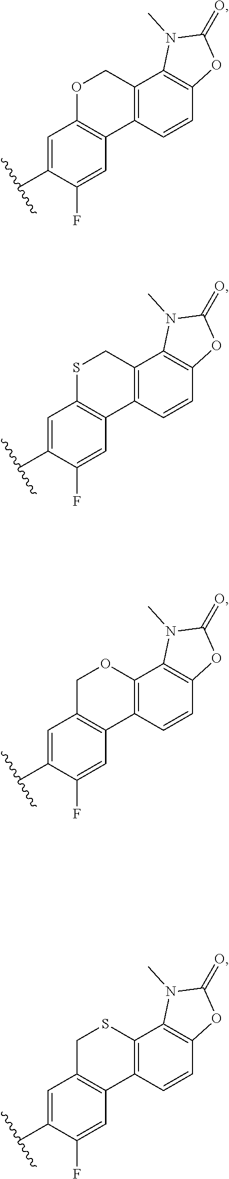 [SMILES: *c1cc2c(cc1F)-c1ccc3oc(=O)n(C)c3c1CO2 ; *c1cc2c(cc1F)-c1ccc3oc(=O)n(C)c3c1CS2 ; *c1cc2c(cc1F)-c1ccc3oc(=O)n(C)c3c1OC2 ; *c1cc2c(cc1F)-c1ccc3oc(=O)n(C)c3c1SC2]

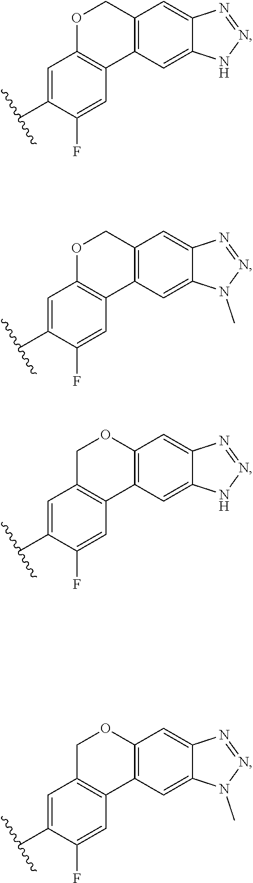 [SMILES: *c1cc2c(cc1F)-c1cc3[nH]nnc3cc1CO2 ; *c1cc2c(cc1F)-c1cc3c(cc1CO2)nnn3C ; *c1cc2c(cc1F)-c1cc3[nH]nnc3cc1OC2 ; *c1cc2c(cc1F)-c1cc3c(cc1OC2)nnn3C]

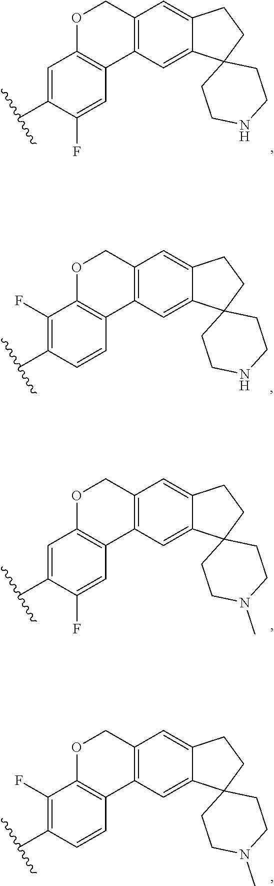 [SMILES: *c1cc2c(cc1F)-c1cc3c(cc1CO2)CCC31CCNCC1 ; *c1ccc2c(c1F)OCc1cc3c(cc1-2)C1(CCNCC1)CC3 ; *c1cc2c(cc1F)-c1cc3c(cc1CO2)CCC31CCN(C)CC1 ; *c1ccc2c(c1F)OCc1cc3c(cc1-2)C1(CC3)CCN(C)CC1]

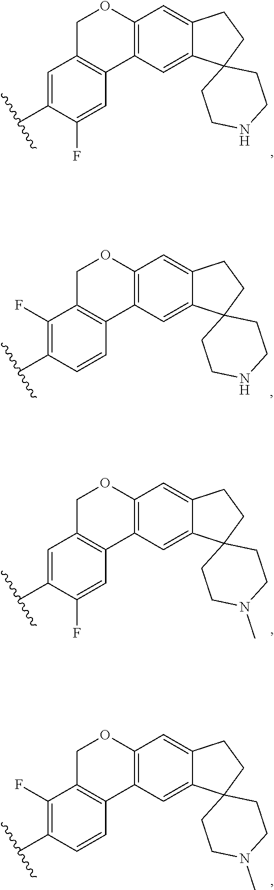 [SMILES: *c1cc2c(cc1F)-c1cc3c(cc1OC2)CCC31CCNCC1 ; *c1ccc2c(c1F)COc1cc3c(cc1-2)C1(CCNCC1)CC3 ; *c1cc2c(cc1F)-c1cc3c(cc1OC2)CCC31CCN(C)CC1 ; *c1ccc2c(c1F)COc1cc3c(cc1-2)C1(CC3)CCN(C)CC1]

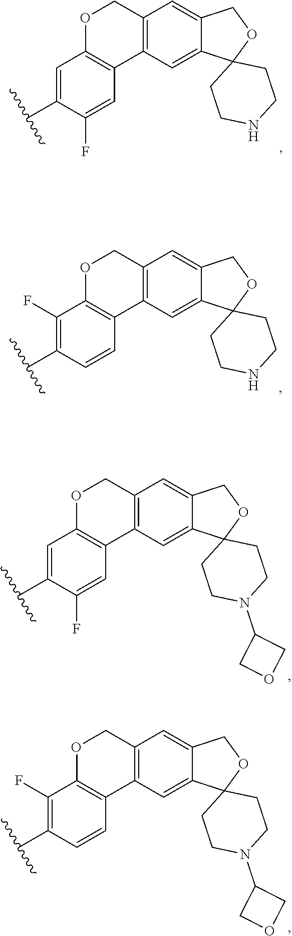 [SMILES: *c1cc2c(cc1F)-c1cc3c(cc1CO2)COC31CCNCC1 ; *c1ccc2c(c1F)OCc1cc3c(cc1-2)C1(CCNCC1)OC3 ; *c1cc2c(cc1F)-c1cc3c(cc1CO2)COC31CCN(C2COC2)CC1 ; *c1ccc2c(c1F)OCc1cc3c(cc1-2)C1(CCN(C2COC2)CC1)OC3]

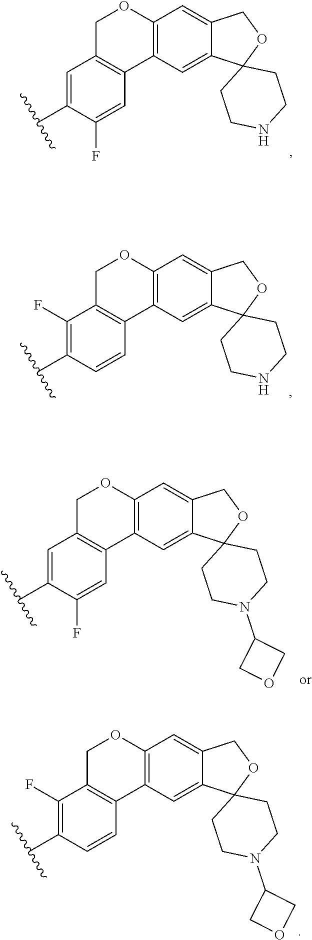 [SMILES: *c1cc2c(cc1F)-c1cc3c(cc1OC2)COC31CCNCC1 ; *c1ccc2c(c1F)COc1cc3c(cc1-2)C1(CCNCC1)OC3 ; *c1cc2c(cc1F)-c1cc3c(cc1OC2)COC31CCN(C2COC2)CC1 ; *c1ccc2c(c1F)COc1cc3c(cc1-2)C1(CCN(C2COC2)CC1)OC3]

In embodiments of the compounds of formula (III) or (III″), or a pharmaceutically acceptable salt, a stereoisomer, or a deuterated form thereof, B is

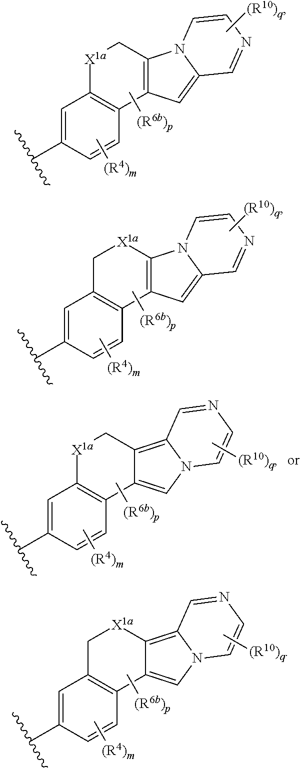

In embodiments, B is

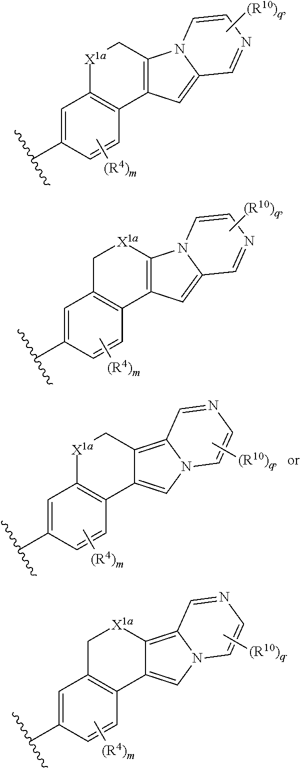

In embodiments, X^{1a} is —O—. In embodiments, m is 0 or 1. In embodiments, R⁴ is halogen. In embodiments, R⁴ is F. In embodiments, p is 0. In embodiments, q is 1 or 2. In embodiments, q is 2. In embodiments, R¹⁰ is hydrogen or methyl.

In embodiments of the compounds of formula (III) or (III″), or a pharmaceutically acceptable salt, a stereoisomer, or a deuterated form thereof, B is

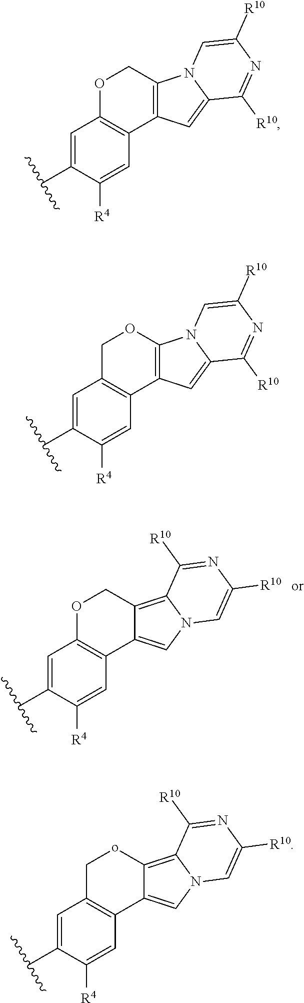

In embodiments, R⁴ is halogen. In embodiments, R⁴ is F. In embodiments, R¹⁰ is hydrogen or methyl.

In embodiments of the compounds of formula (III) or (III″), or a pharmaceutically acceptable salt, a stereoisomer, or a deuterated form thereof, B is

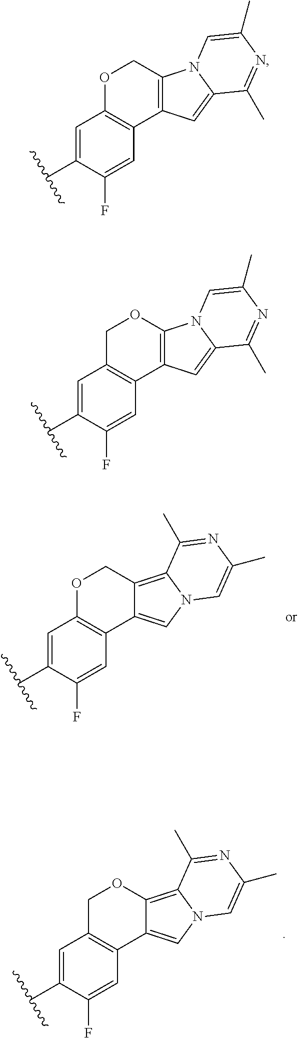 [SMILES: *c1cc2c(cc1F)-c1cc3c(C)nc(C)cn3c1CO2 ; *c1cc2c(cc1F)-c1cc3c(C)nc(C)cn3c1OC2 ; *c1cc2c(cc1F)-c1cn3cc(C)nc(C)c3c1CO2 ; *c1cc2c(cc1F)-c1cn3cc(C)nc(C)c3c1OC2]

In embodiments, B is

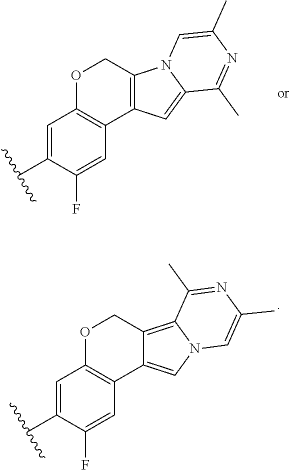 [SMILES: *c1cc2c(cc1F)-c1cc3c(C)nc(C)cn3c1CO2 ; *c1cc2c(cc1F)-c1cn3cc(C)nc(C)c3c1CO2]

In embodiments of the compounds of formula (III) or (III′), or a pharmaceutically acceptable salt, a stereoisomer, or a deuterated form thereof, the compound has the structure of formula (III-A):

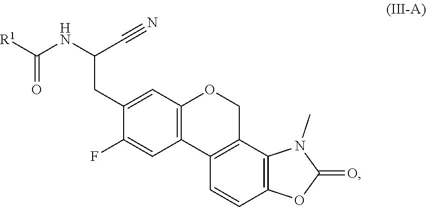

or a pharmaceutically acceptable salt, a stereoisomer, or a deuterated form thereof wherein:
R¹ is

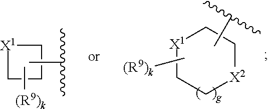

each R⁹ is independently halogen, —OH, —C₁-C₆ alkyl, —C₁-C₆ alkyl-OH, or —OC₁-C₆ alkyl;
X¹ is —O—, —S—, or —NH—;
X² is —O—, —S—, —NH—, or —CR¹¹R¹²—;
R¹¹ and R¹² are each independently hydrogen, halogen, or —C₁-C₆ alkyl;
g is 1, 2, or 3; and
k is 0, 1, or 2.

In embodiments of the compounds of formula (III) or (III-A), or a pharmaceutically acceptable salt, a stereoisomer, or a deuterated form thereof,
R¹ is

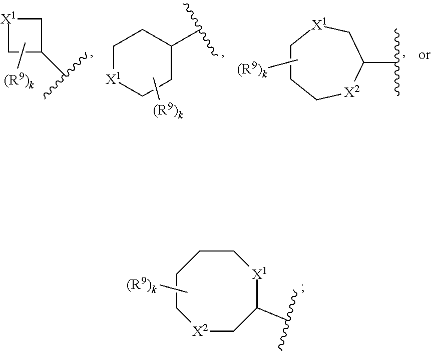

X¹ is —O—, —S—, or —NH—;
X² is —O—, —S—, —NH—, or —CH₂—; and
each R⁹ is independently —OH, fluoro, methyl, ethyl, —CH₂OH, or —OCH₃.

In embodiments of the compounds of formula (III), (III′), (III″), or (III-A), or a pharmaceutically acceptable salt, a stereoisomer, or a deuterated form thereof, R¹ is

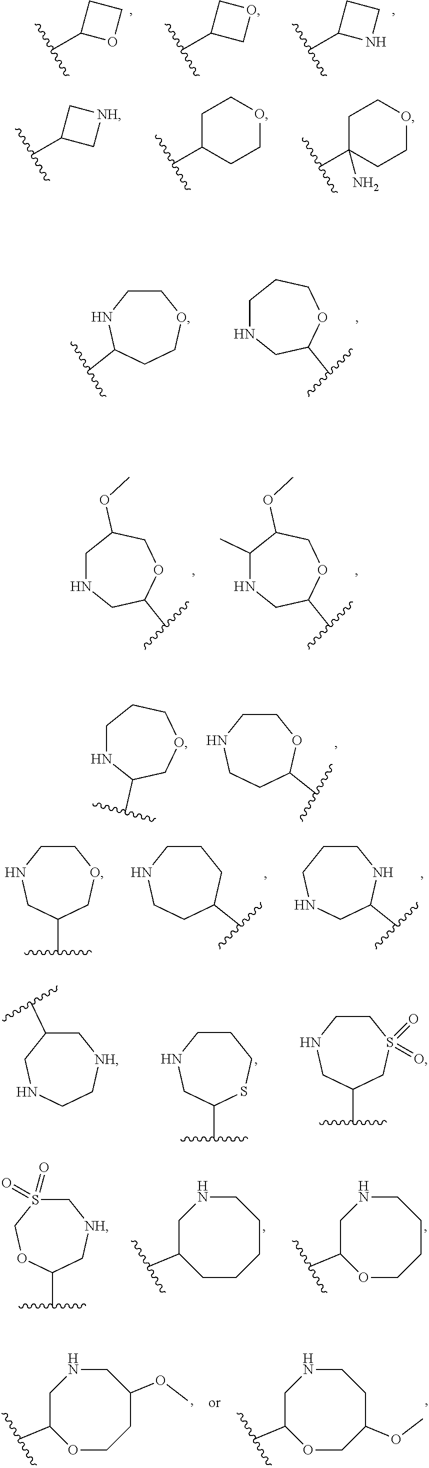

each of which is optionally substituted with 1 or 2 R⁹. In embodiments, R¹ is

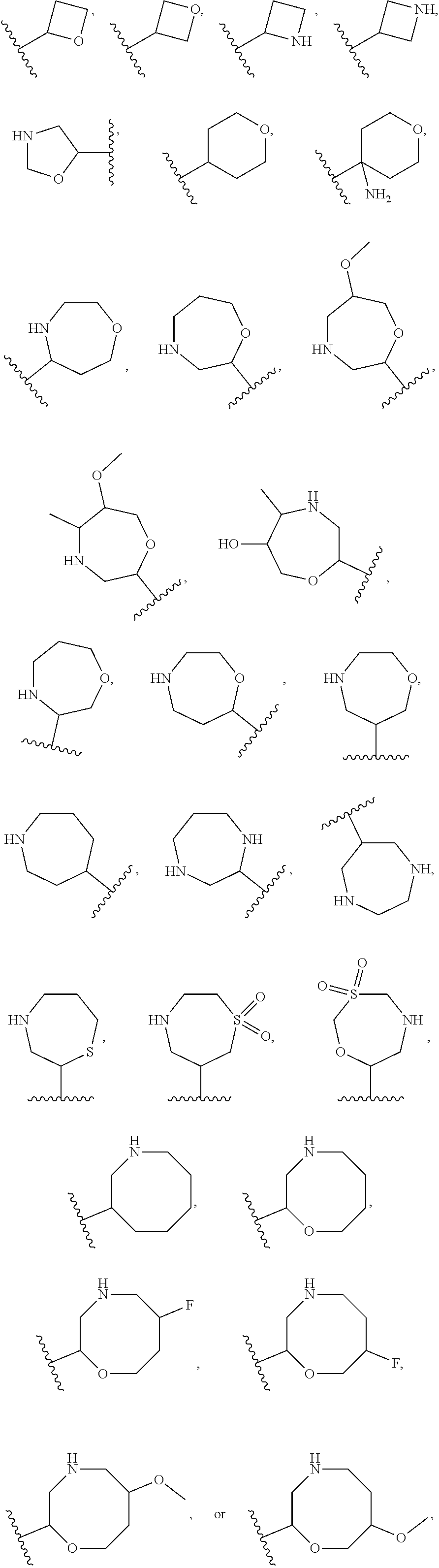

each of which is optionally substituted with 1 or 2 R⁹. In embodiments, R¹ is

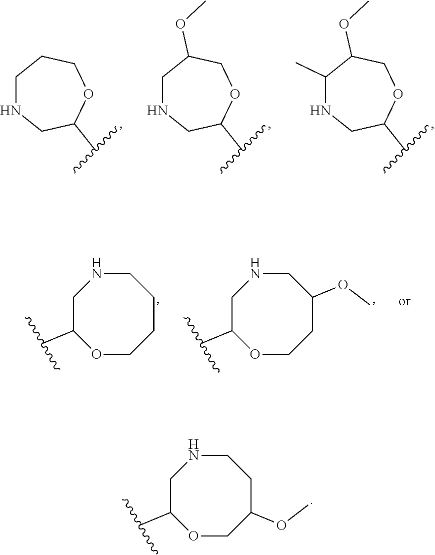

In embodiments, R¹ is

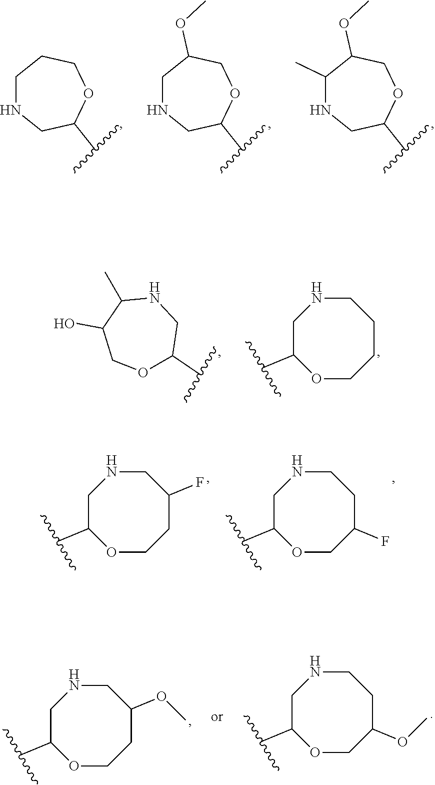

In embodiments of the compounds of formula (III), or a pharmaceutically acceptable salt, a stereoisomer, or a deuterated form thereof, B is

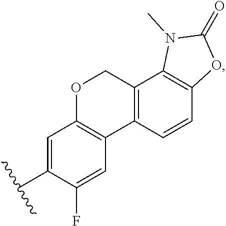 [SMILES: *c1cc2c(cc1F)-c1ccc3oc(=O)n(C)c3c1CO2]

### and R¹ is

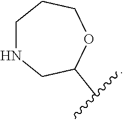 [SMILES: *C1CNCCCO1]

Embodiments provided herein for formula (III) or (III′) can be applied for formula (III-A), if appropriate under the definitions of formula (III-A).

In embodiments, the present disclosure provides a compound of formula (IV):

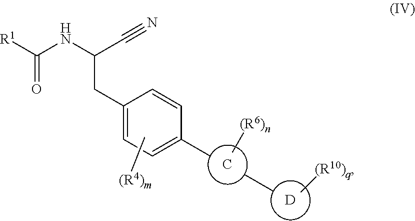

or a pharmaceutically acceptable salt, a stereoisomer, or a deuterated form thereof, wherein:
ring C is a monocyclic heteroaryl ring;
ring D is a monocyclic heteroaryl or a monocyclic heterocyclyl ring;
R¹ is

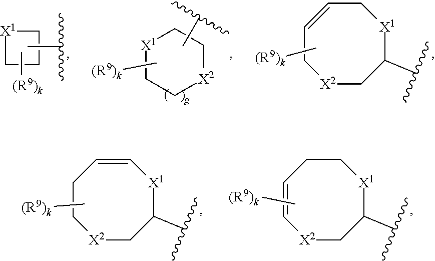

or C₁-C₆ alkylene(NH)-carbocycle substituted with 0, 1, 2, 3, or 4 R³;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
g is 0, 1, 2, or 3;
k is 0, 1, 2, 3, or 4;
m is 0, 1, 2, or 3;
n is 0, 1, 2, 3, or 4; and
q is 0, 1, 2, or 3.

In embodiments of the compounds of formula (IV), or a pharmaceutically acceptable salt, a stereoisomer, or a deuterated form thereof, ring C is a 5-membered heteroaryl ring. In embodiments, ring C is furan, thiophene, pyrazole, oxazole, or thiazole. In embodiments, ring C is

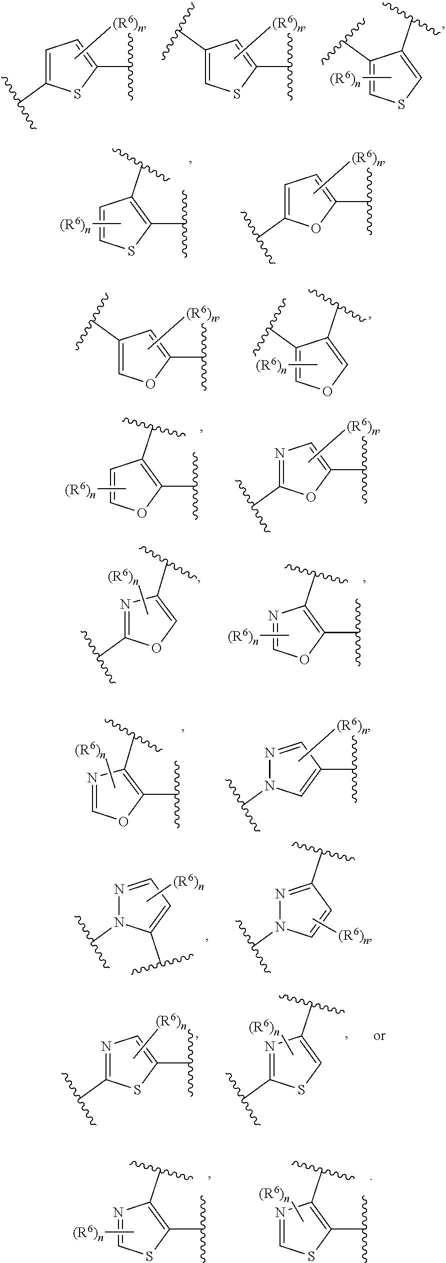

In embodiments, n is 0 or 1. In embodiments, R⁶ is methyl or ethyl.

In embodiments of the compounds of formula (IV), or a pharmaceutically acceptable salt, a stereoisomer, or a deuterated form thereof, ring C is

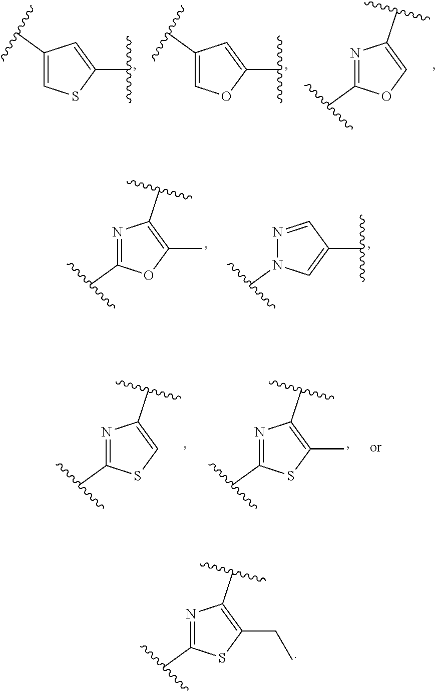

In embodiments of the compounds of formula (IV), or a pharmaceutically acceptable salt, a stereoisomer, or a deuterated form thereof, ring D is a 5- or 6-membered heteroaryl or heterocyclyl ring. In embodiments, ring D is tetrahydrofuran, tetrahydropyran, dioxolane, oxazole, thiazole, pyrazole, oxadiazole, thiadiazole, or dihydropyridine.

In embodiments of the compounds of formula (IV), or a pharmaceutically acceptable salt, a stereoisomer, or a deuterated form thereof, ring D is

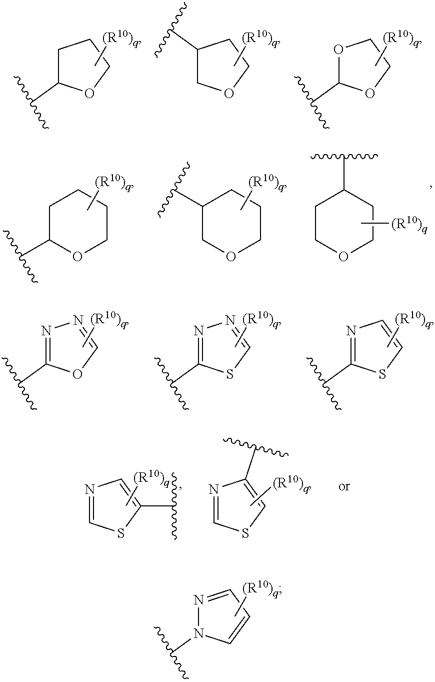

wherein q is 0, 1, or 2. In embodiments, q is 0 or 1. In embodiments, R¹⁰ is —NH₂.

In embodiments of the compounds of formula (IV), or a pharmaceutically acceptable salt, a stereoisomer, or a deuterated form thereof, ring D is

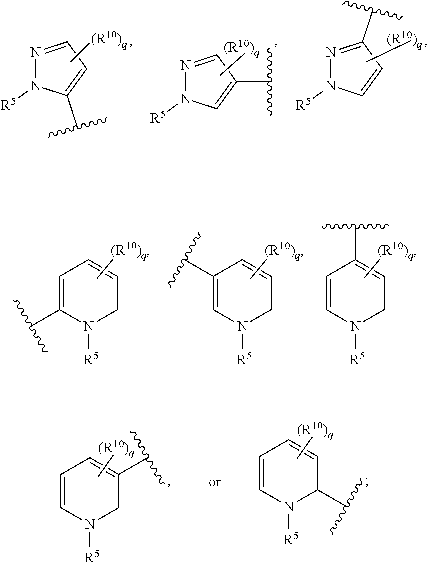

wherein q is 0, 1, or 2; and R⁵ is hydrogen or —C₁-C₆ alkyl. In embodiments, q is 0 or 1. In embodiments, R⁵ is hydrogen or methyl. In embodiments, R¹⁰ is oxo.

In embodiments of the compounds of formula (IV), or a pharmaceutically acceptable salt, a stereoisomer, or a deuterated form thereof, ring D is

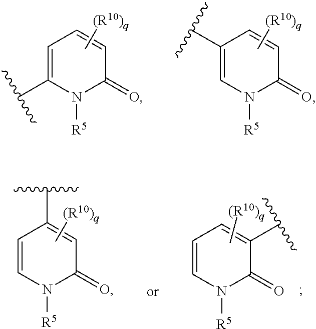

wherein q is 0 or 1. In embodiments, R⁵ is hydrogen or —C₁-C₆ alkyl. In embodiments, R⁵ is hydrogen or methyl. In embodiments, q is 0.

In embodiments of the compounds of formula (IV), or pharmaceutically acceptable salt, a stereoisomer, or a deuterated form thereof, ring D is

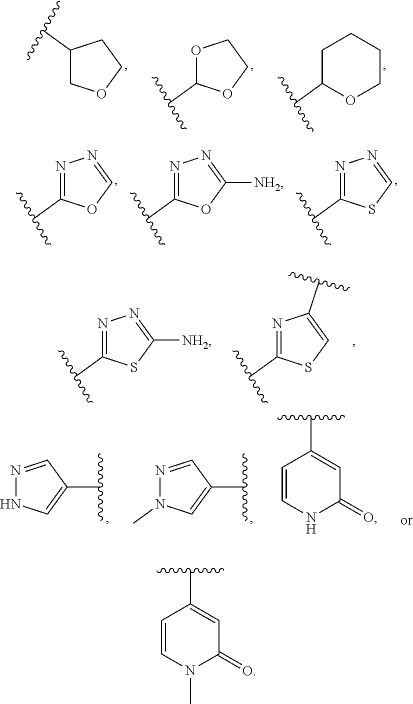

In embodiments of the compounds of formula (IV), or a pharmaceutically acceptable salt, a stereoisomer, or a deuterated form thereof, R¹⁰ is oxo, —C₁-C₃ alkyl, or —NH₂,

In embodiments of the compounds of formula (I), or a pharmaceutically acceptable salt, a stereoisomer, or a deuterated form thereof, wherein:
R¹ is

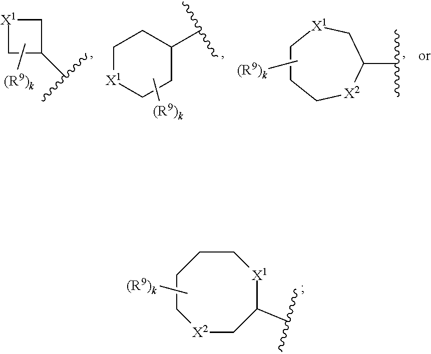

X¹ is —O—, —S—, or —NH—;
X² is —O—, —S—, —NH—, or —CH₂—; and
each R⁹ is independently —OH, fluoro, methyl, ethyl, —CH₂OH, or —OCH₃.

In embodiments of the compounds of formula (IV), or a pharmaceutically acceptable salt, a stereoisomer, or a deuterated form thereof, R¹ is

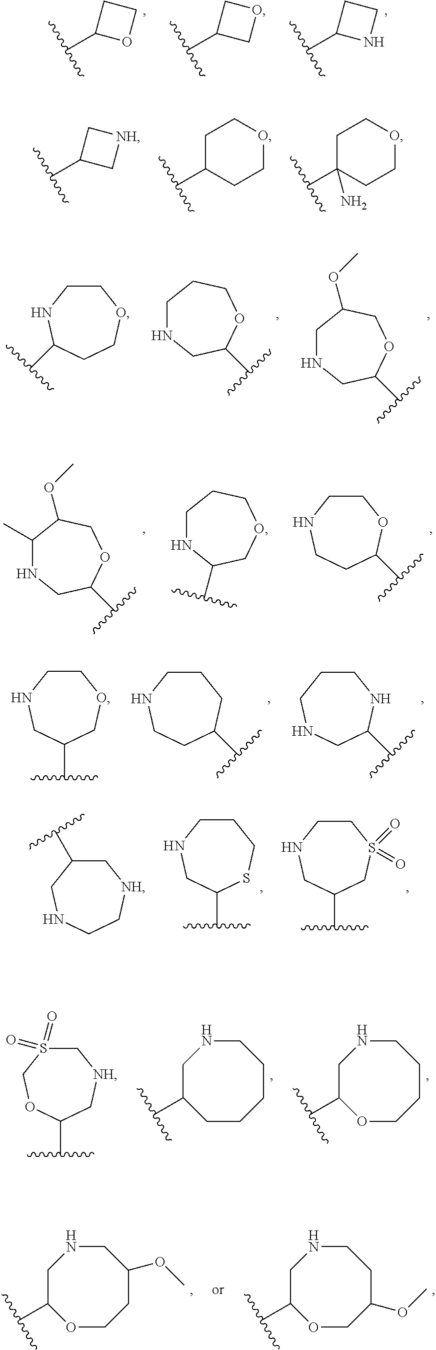

each of which is optionally substituted with 1, 2, 3 or 4 R⁹. In embodiments, R¹ is

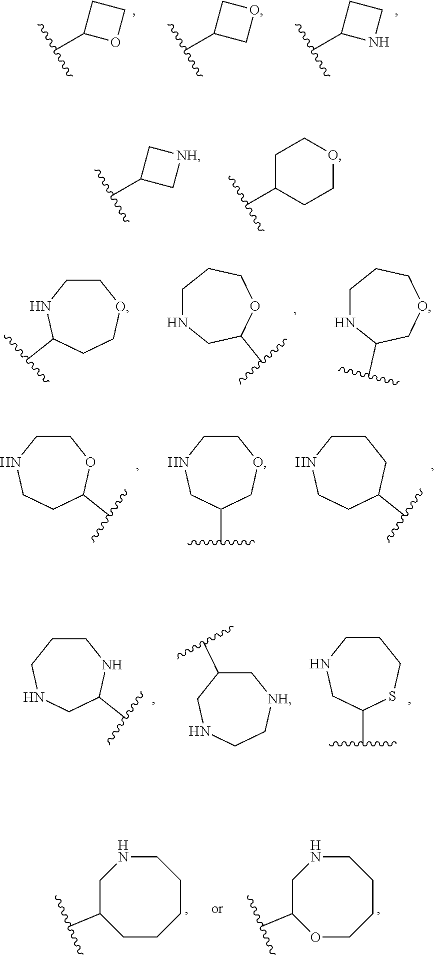

each of which is optionally substituted with 1, 2, 3, or 4 R⁹. In embodiments, R¹ is

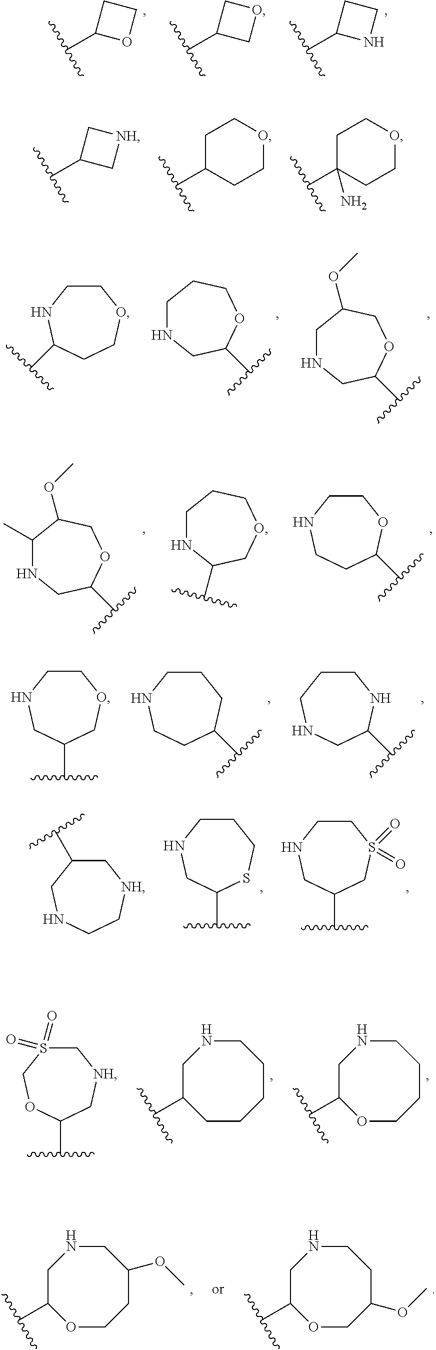

In embodiments, R¹ is

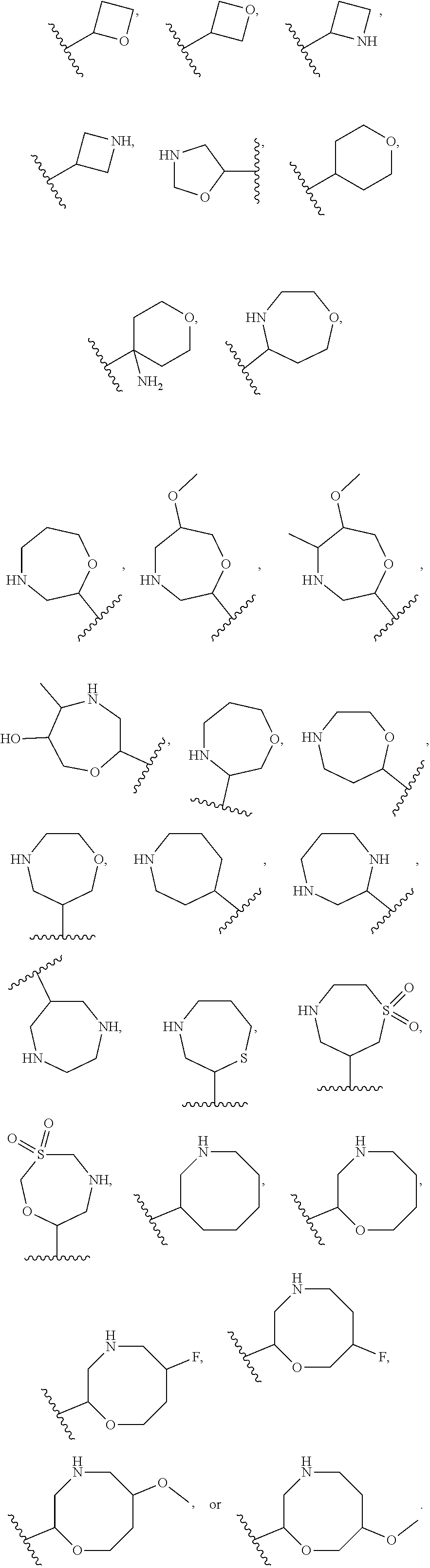

In embodiments, R¹ is

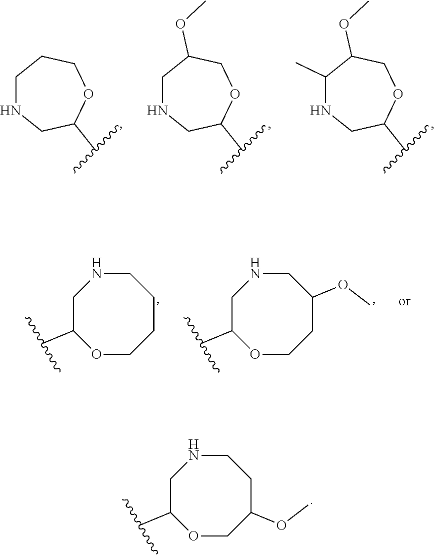

In embodiments, R¹ is

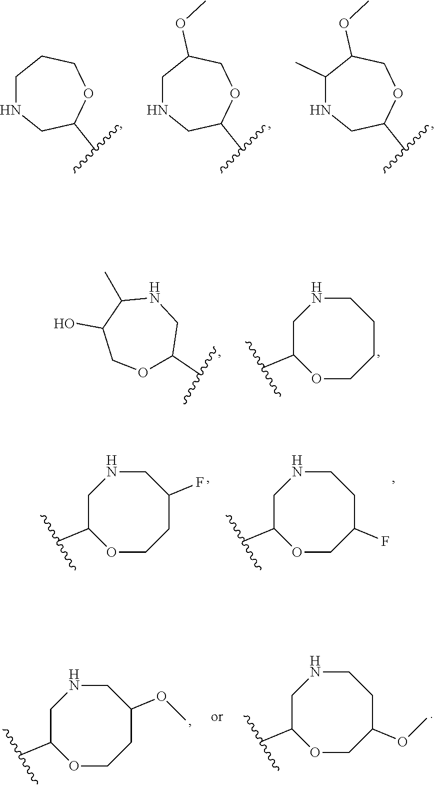

In embodiments of the compounds of formula (IV), or a pharmaceutically acceptable salt, a stereoisomer, or a deuterated form thereof, m is 0 or 1 and R⁴ is a halogen. In embodiments, R⁴ is F.

In embodiments, the present disclosure provides a compound of formula (V):

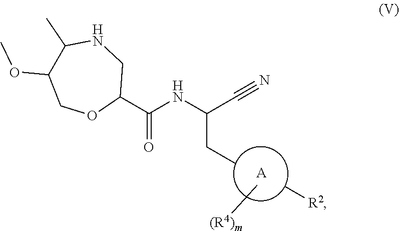

or a pharmaceutically acceptable salt, a stereoisomer, or a deuterated form thereof, wherein:
ring A is a carbocycle, aryl, heterocycle, or heteroaryl ring;
R² is monocyclic, bicyclic, or tricyclic ring, wherein R² is optionally substituted with 1, 2, 3, or 4 R⁵, and optionally substituted with 1, 2, 3, or 4 R⁶, wherein each R⁵ is bound to a ring nitrogen;
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, or —(C₁-C₆ alkylene)-O—(C₁-C₆ alkyl), or R⁷ and R⁸ together with the nitrogen atom to which they are attached form a heterocyclyl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle; and
m is 0, 1, 2, or 3.

In embodiments of the compounds of formula (V), or a pharmaceutically acceptable salt, a stereoisomer, or a deuterated form thereof, R² is a bicyclic ring, optionally substituted with 1, 2, 3, or 4 R⁵, and optionally substituted with 1, 2, 3, or 4 R⁶, wherein each R⁵ is bound to a ring nitrogen. In embodiments, R² is a fused or spiral ring.

In embodiments of the compounds of formula (V), or a pharmaceutically acceptable salt, a stereoisomer, or a deuterated form thereof,
R² is

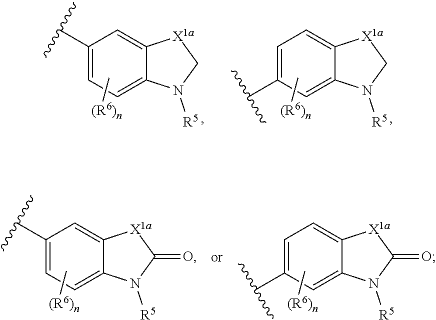

each X^{1a} is independently —NR⁵—, —O—, —CR¹¹R¹²—, —C(O)—, —S—, —S(O)— or —S(O)₂—;
each R⁵ is independently H, —C₁-C₆ alkyl, —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, or —(C₁-C₆ alkylene)-heterocycle;
each R⁶ is independently halogen, —OH, —CN, —C₁-C₆ alkyl, or —C₁-C₆ alkyl-OH;
R⁷ and R⁸ are each independently hydrogen, C₁₋₆ alkyl, or —(C₁-C₆ alkylene)-O—(C₁-C₆ alkyl);
R¹¹ and R¹² are each independently hydrogen, halogen, or —C₁-C₆ alkyl; and
n is 0, 1, 2, or 3.

In embodiments of the compounds of formula (V), or a pharmaceutically acceptable salt, a stereoisomer, or a deuterated form thereof, R² is

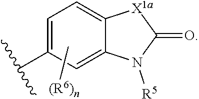

In embodiments of the compounds of formula (V), or a pharmaceutically acceptable salt, a stereoisomer, or a deuterated form thereof,
R² is

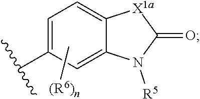

X^{1a} is —O— or —CR¹¹R¹²—;
R⁵ is —C₁-C₆ alkyl, —(C₁-C₆ alkylene)-O—(C₁-C₆ alkyl), —C₁-C₆ alkyl-NR⁷R⁸;
R⁷ and R⁸ are each independently C₁₋₆ alkyl, or —(C₁-C₆ alkylene)-O—(C₁-C₆ alkyl);
R¹¹ and R¹² are each independently —C₁-C₆ alkyl; and
n is 0.

In embodiments of the compounds of formula (V), or a pharmaceutically acceptable salt, a stereoisomer, or a deuterated form thereof, R² is

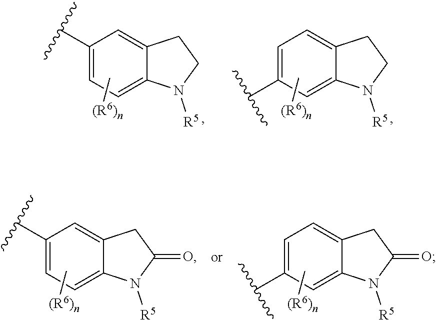

and p is 0 or 1. In embodiments, R² is

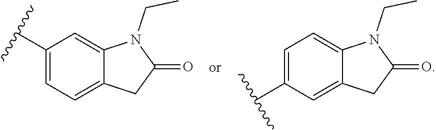 [SMILES: *c1ccc2c(c1)N(CC)C(=O)C2 ; *c1ccc2c(c1)CC(=O)N2CC]

In embodiments of the compounds of formula (V), or a pharmaceutically acceptable salt, a stereoisomer, or a deuterated form thereof, R² is

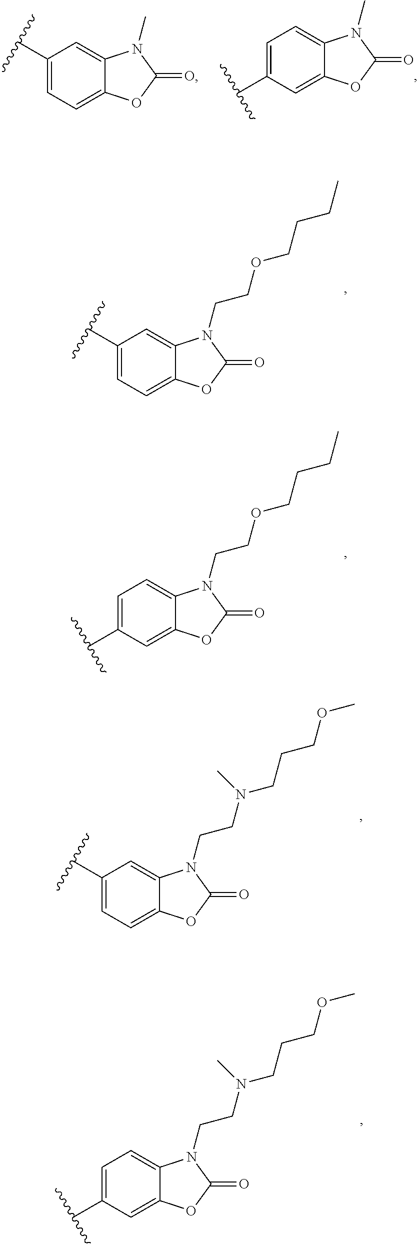 [SMILES: *c1ccc2oc(=O)n(C)c2c1 ; *c1ccc2c(c1)oc(=O)n2C ; *c1ccc2oc(=O)n(CCOCCCC)c2c1 ; *c1ccc2c(c1)oc(=O)n2CCOCCCC ; *c1ccc2oc(=O)n(CCN(C)CCCOC)c2c1 ; *c1ccc2c(c1)oc(=O)n2CCN(C)CCCOC]

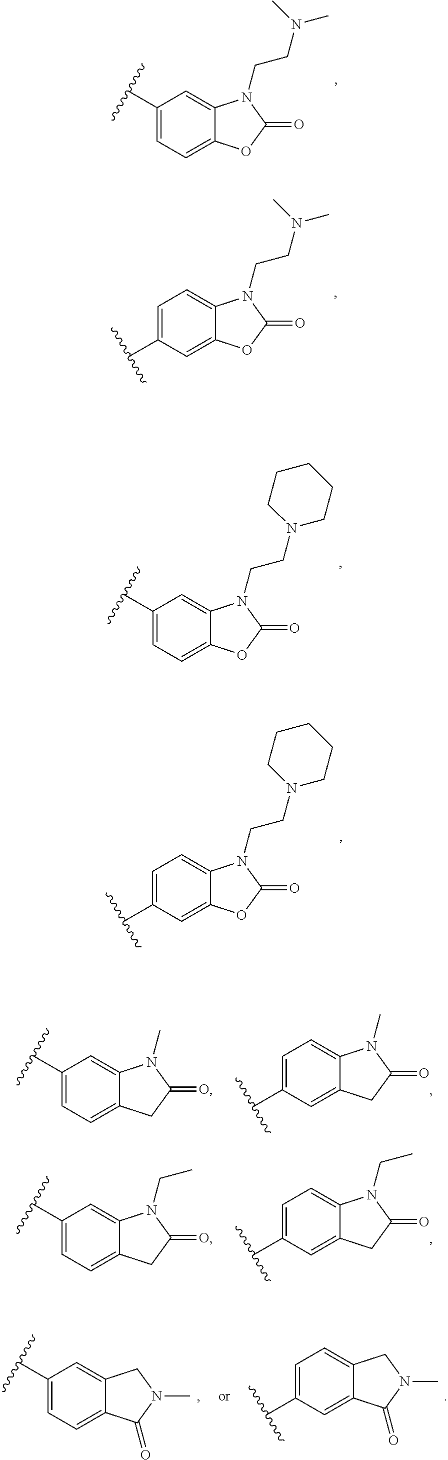

In embodiments, R² is not

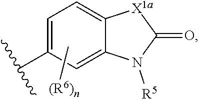

wherein X^{1a} is —O—. In embodiments, R² is not,

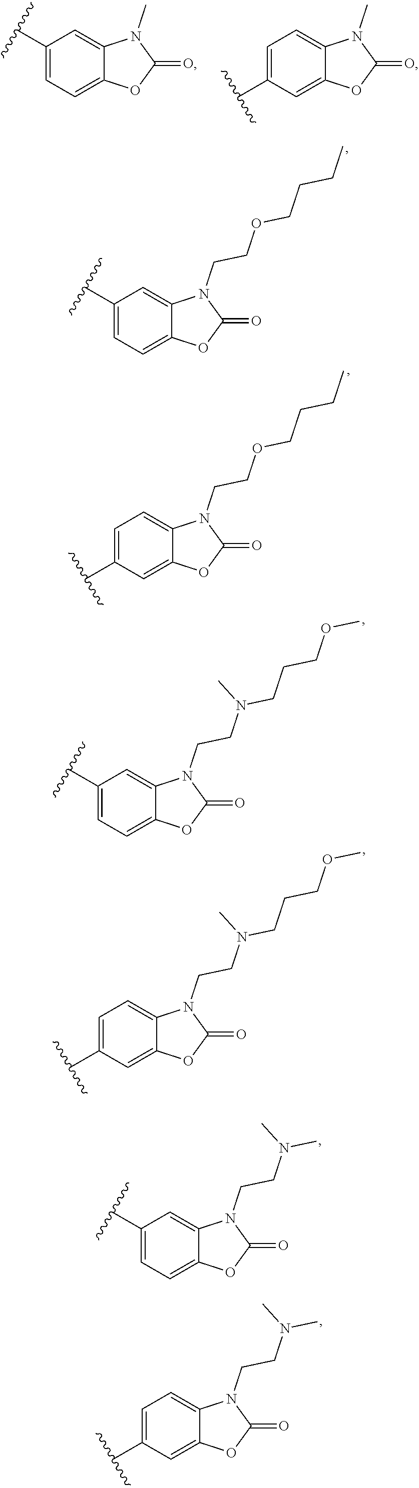

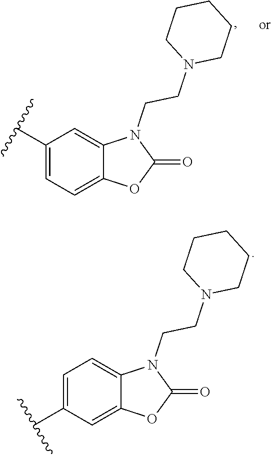 [SMILES: *c1ccc2oc(=O)n(CCN3CCCCC3)c2c1 ; *c1ccc2c(c1)oc(=O)n2CCN1CCCCC1]

In embodiments of the compounds of formula (V), or a pharmaceutically acceptable salt, a stereoisomer, or a deuterated form thereof, R² is

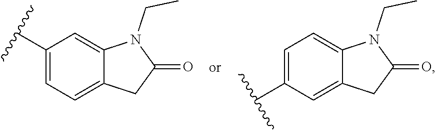 [SMILES: *c1ccc2c(c1)N(CC)C(=O)C2 ; *c1ccc2c(c1)CC(=O)N2CC]

In embodiments of the compounds of formula (V), or a pharmaceutically acceptable salt, a stereoisomer, or a deuterated form thereof, R² is

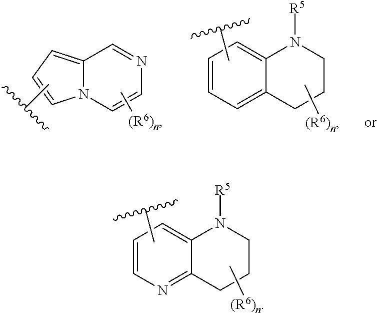

In embodiments, R² is

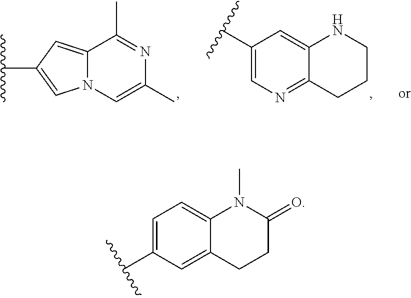 [SMILES: *c1cc2c(C)nc(C)cn2c1 ; *c1cnc2c(c1)NCCC2 ; *c1ccc2c(c1)CCC(=O)N2C]

In embodiments of the compounds of formula (V), or a pharmaceutically acceptable salt, a stereoisomer, or a deuterated form thereof, R² is tricyclic ring, optionally substituted with 1, 2, 3, or 4 R⁵, and optionally substituted with 1, 2, 3, or 4 R⁶, wherein each R⁵ is bound to a ring nitrogen.

In embodiments of the compounds of formula (V), or a pharmaceutically acceptable salt, a stereoisomer, or a deuterated form thereof, R² is

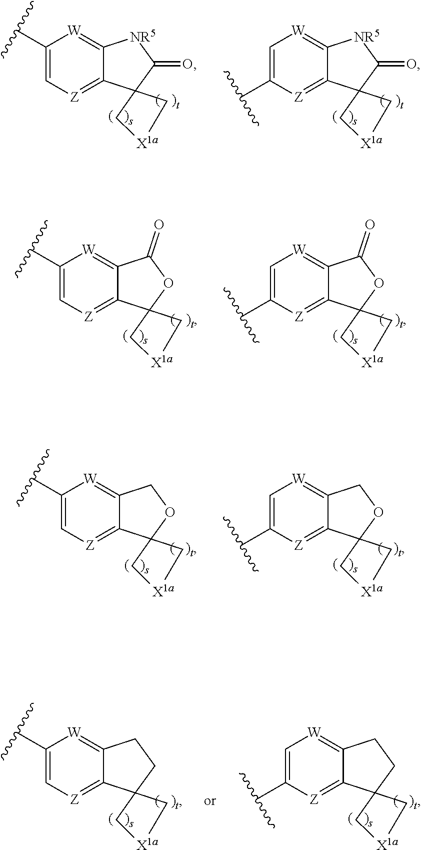

wherein R² is optionally substituted with 1 or 2 R⁶. In one embodiment, W and Z are both CH or W is CH and Z is N. In embodiments, X^{1a} is —NR⁵—. In embodiments, R⁵ is H, —C₁-C₆ alkyl, or 4-6 membered heterocycle. In embodiments, R⁵ is H, methyl or

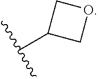 [SMILES: *C1COC1]

In embodiments, a) s is 2 and t is 2; b) s is 1 and t is 3 or s is 3 and t is 1; c) s is 1 and t is 2 or s is 2 and t is 1; or d) s is 1 and t is 1.

In embodiments, R² is

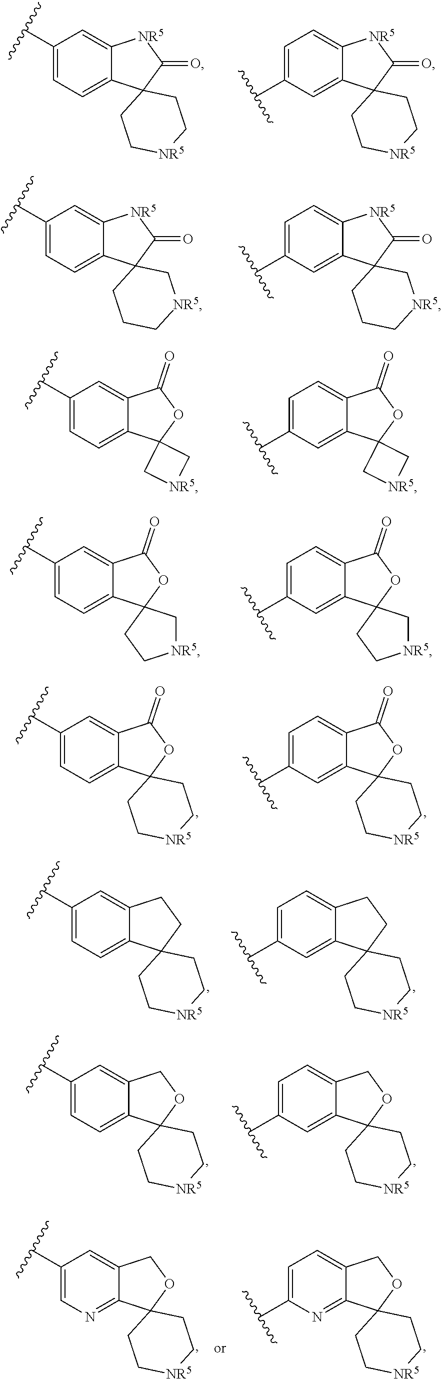

wherein each R² is optionally substituted with 1 or 2 R⁶. In embodiments, R² is

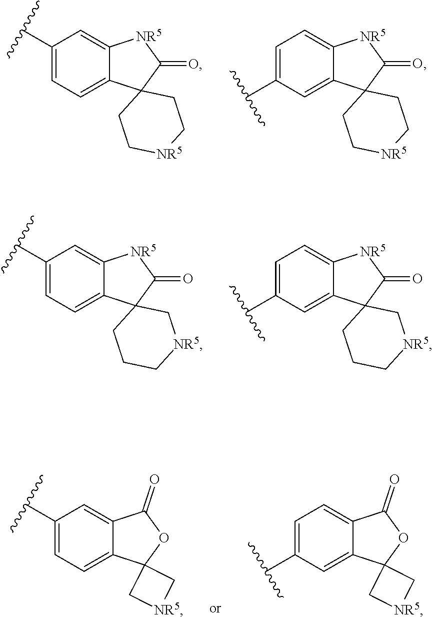

wherein each R² is optionally substituted with 1 or 2 R⁶. In embodiments, R² is

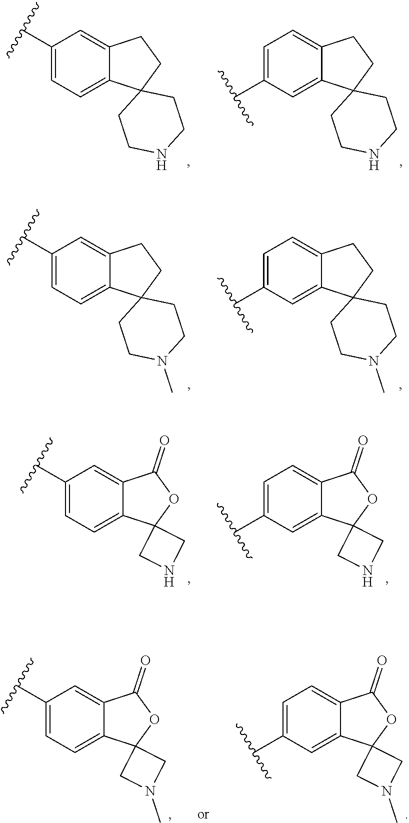

In embodiments of the compounds of formula (V), or a pharmaceutically acceptable

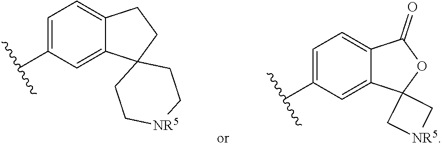

salt, a stereoisomer, or a deuterated form thereof, R² is

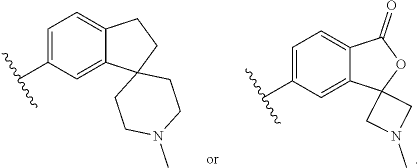 [SMILES: *c1ccc2c(c1)C1(CC2)CCN(C)CC1 ; *c1ccc2c(c1)C1(CN(C)C1)OC2=O]

In embodiments, R² is

In embodiments of the compounds of formula (V), or a pharmaceutically acceptable salt, a stereoisomer, or a deuterated form thereof, R² is

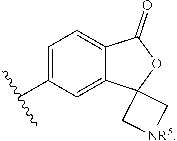

In embodiments, R² is

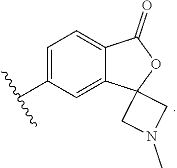 [SMILES: *c1ccc2c(c1)C1(CN(C)C1)OC2=O]

In embodiments of the compounds of formula (V), or a pharmaceutically acceptable salt, a stereoisomer, or a deuterated form thereof, R² is not

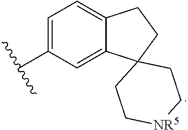

In embodiments, R² is not

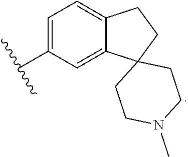 [SMILES: *c1ccc2c(c1)C1(CC2)CCN(C)CC1]

In embodiments of the compounds of formula (V), or a pharmaceutically acceptable salt, a stereoisomer, or a deuterated form thereof, R² is a monocyclic, bicyclic, or tricyclic ring, wherein R² is optionally substituted with 1, 2, 3, or 4 R⁶. In one embodiment, R² is a monocyclic carbocycle, monocyclic aryl, monocyclic heterocycle, or monocyclic heteroaryl, wherein R² is optionally substituted with 1, 2, 3, or 4 R⁶. In one embodiment, R² is phenyl, optionally substituted with 1, 2, 3, or 4 R⁶.

In embodiments of the compounds of formula (V), or a pharmaceutically acceptable salt, a stereoisomer, or a deuterated form thereof, R² is a bicyclic carbocycle, bicyclic aryl, bicyclic heterocycle, or bicyclic heteroaryl, wherein R² is optionally substituted with 1, 2, 3, or 4 R⁶. In one embodiment, R² is a fused ring or a spiral ring.

In embodiments of the compounds of formula (V), or a pharmaceutically acceptable salt, a stereoisomer, or a deuterated form thereof, R² is aryl, wherein R² is optionally substituted with 1, 2, 3, or 4 R⁶. In embodiments, R² is phenyl, wherein R² is optionally substituted with 1, 2, 3, or 4 R⁶. In embodiments, R² is

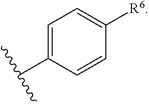

In embodiments, R² is

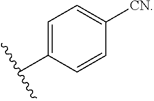 [SMILES: *c1ccc(C#N)cc1]

In embodiments of the compounds of formula (V), or a pharmaceutically acceptable salt, a stereoisomer, or a deuterated form thereof, R⁵ is —C₁-C₆ alkyl, —CH₂CH₂OCH₂CH₃, —CH₂CH₂OCH₂CH₂CH₃, —CH₂CH₂OCH₂CH₂CH₂CH₃, —CH₂CH₂OCH₂CH₂OH, —CH₂CH₂OCH₂CH₂OCH₃, —CH₂CH₂N(CH₃)₂, or

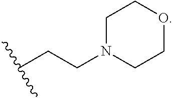 [SMILES: *CCN1CCOCC1]

In embodiments of the compounds of formula (V), or a pharmaceutically acceptable salt, a stereoisomer, or a deuterated form thereof, the compound has the structure of formula (V-A):

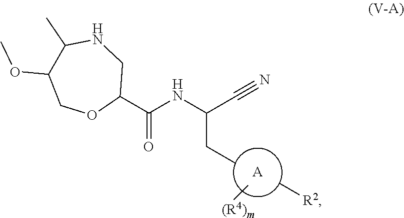

or a pharmaceutically acceptable salt, a stereoisomer, or a deuterated form thereof wherein:
ring A is an aryl or heteroaryl ring;
R² is

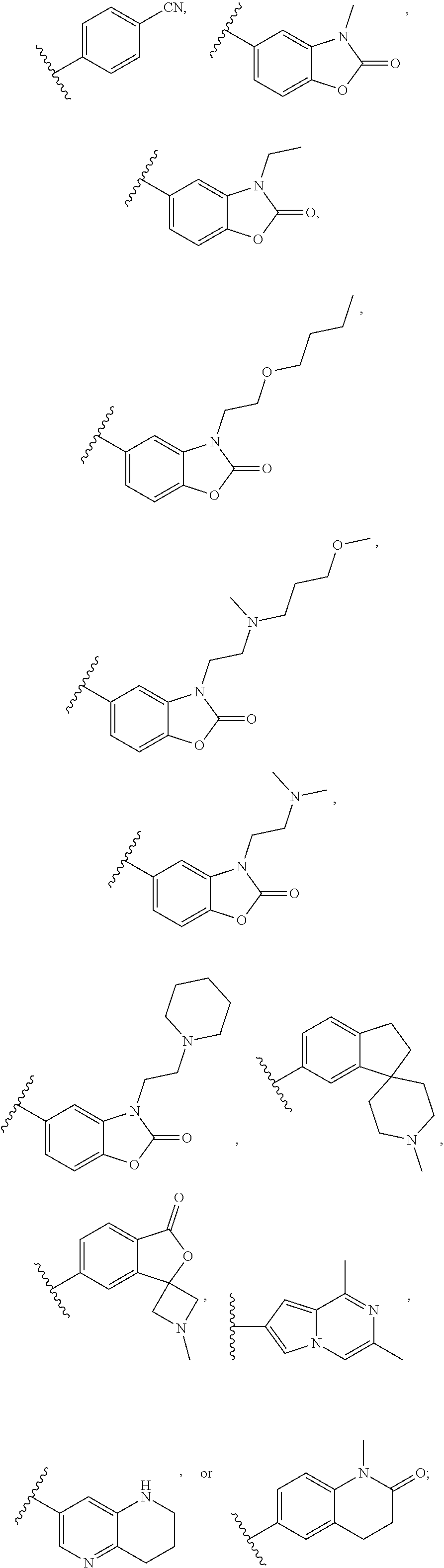

each R⁴ is independently halogen, —C₁-C₆ alkyl, —C₁-C₆ haloalkyl, or —CN; and
m is 0, 1, or 2.

In embodiments of the compounds of formula (V) or (V-A), or a pharmaceutically acceptable salt, a stereoisomer, or a deuterated form thereof, R² is or

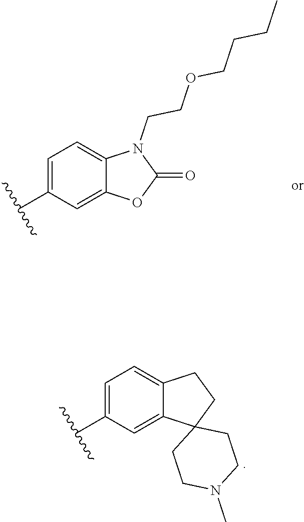 [SMILES: *c1ccc2c(c1)oc(=O)n2CCOCCCC ; *c1ccc2c(c1)C1(CC2)CCN(C)CC1]

In embodiments of the compounds of formula (V) or (V-A), or a pharmaceutically acceptable salt, a stereoisomer, or a deuterated form thereof, A is a monocyclic or a bicyclic ring.

In embodiments of the compounds of formula (V) or (V-A), or a pharmaceutically acceptable salt, a stereoisomer, or a deuterated form thereof, A is aryl or heteroaryl. In embodiments, A is a monocyclic or bicyclic heteroaryl.

In embodiments of the compounds of formula (V) or (V-A), or a pharmaceutically acceptable salt, a stereoisomer, or a deuterated form thereof, A is a phenyl ring. In embodiments, A is

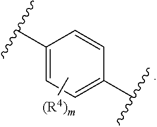

In embodiments, m is 1 or 2 and R⁴ is each independently a halogen. In embodiments, m is 1 or 2 and R⁴ is each F. In embodiments, m is 1 and R⁴ is F. In embodiments, m is 0. In embodiments, A is

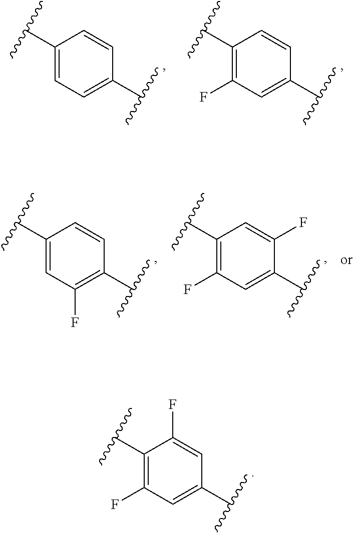 [SMILES: *c1ccc(*)cc1 ; *c1ccc(*)c(F)c1 ; *c1ccc(*)c(F)c1 ; *c1cc(F)c(*)cc1F ; *c1cc(F)c(*)c(F)c1]

In embodiments of the compounds of formula (V) or (V-A), or a pharmaceutically acceptable salt, a stereoisomer, or a deuterated form thereof, A is

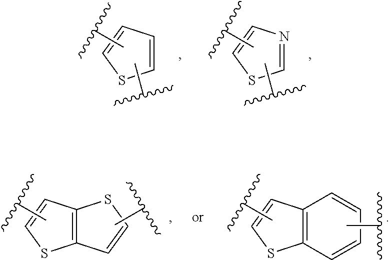

In embodiments, A is

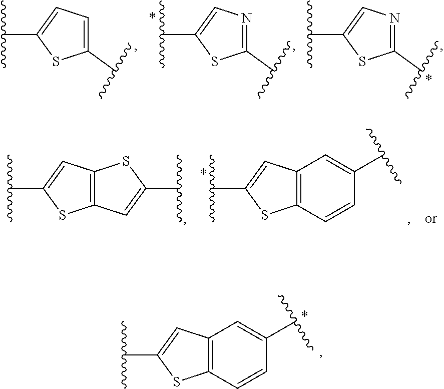

wherein * denotes connectivity to R².

In embodiments of the compounds of formula (V), or a pharmaceutically acceptable salt, a stereoisomer, or a deuterated form thereof, the compound has the structure of formula (V-B):

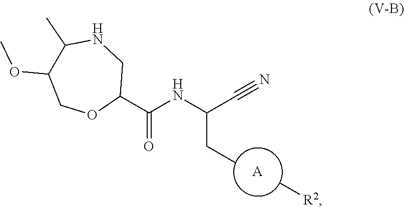

or a pharmaceutically acceptable salt, a stereoisomer, or a deuterated form thereof wherein:
ring A is

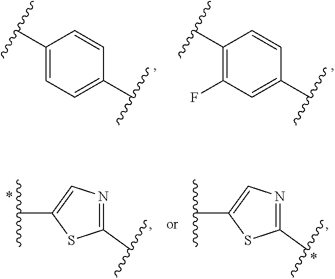 [SMILES: *c1ccc(*)cc1 ; *c1ccc(*)c(F)c1 ; *c1cnc(*)s1 ; *c1cnc(*)s1]

wherein * denotes connectivity to R²; and
R² is

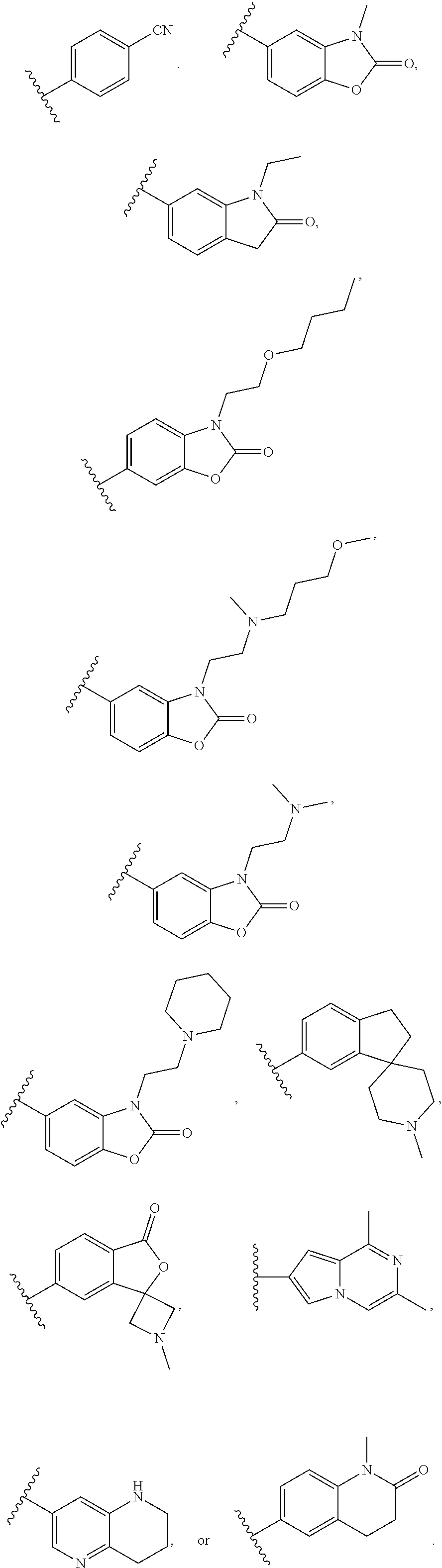

In embodiments of the compounds of formula (V), (V-A), or (V-B), or a pharmaceutically acceptable salt, a stereoisomer, or a deuterated form thereof, R² is

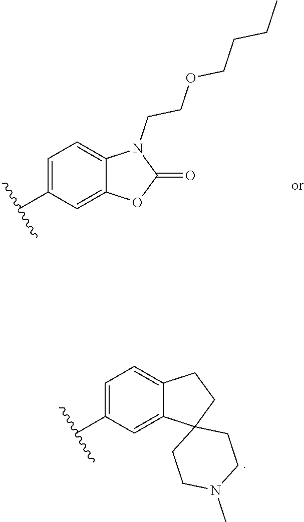 [SMILES: *c1ccc2c(c1)oc(=O)n2CCOCCCC ; *c1ccc2c(c1)C1(CC2)CCN(C)CC1]

Embodiments provided herein for formula (V) can be applied for formula (V-A) and/or (V-B), if appropriate under the definitions of formula (V-A) and/or (V-B).

In embodiments, the present disclosure provides a compound of formula (VI):

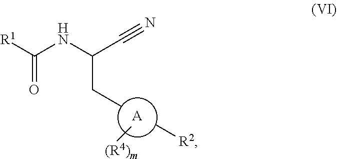

or a pharmaceutically acceptable salt, a stereoisomer, or a deuterated form thereof, wherein:
ring A is a carbocyclyl, aryl, heterocyclyl, or heteroaryl ring;
R¹ is or

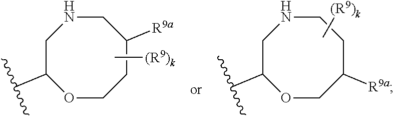

R² is monocyclic, bicyclic, or tricyclic ring, wherein R² is optionally substituted with 1, 2, 3, or 4 R⁵, and optionally substituted with 1, 2, 3, or 4 R⁶, wherein each R⁵ is bound to a ring nitrogen;
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
R^{9a}—O—(C₁-C₆ alkyl);
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
m is 0, 1, 2, or 3;
n is 0, 1, 2, 3, or 4;
each g is independently 0, 1, 2, or 3; and
k is 0, 1, 2, or 3;
wherein the compound is not

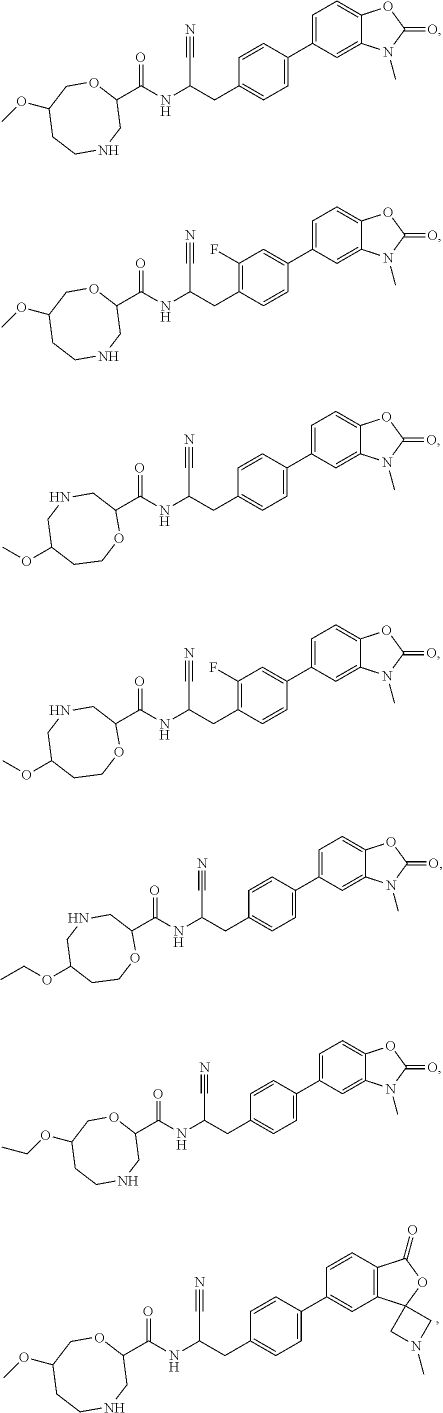

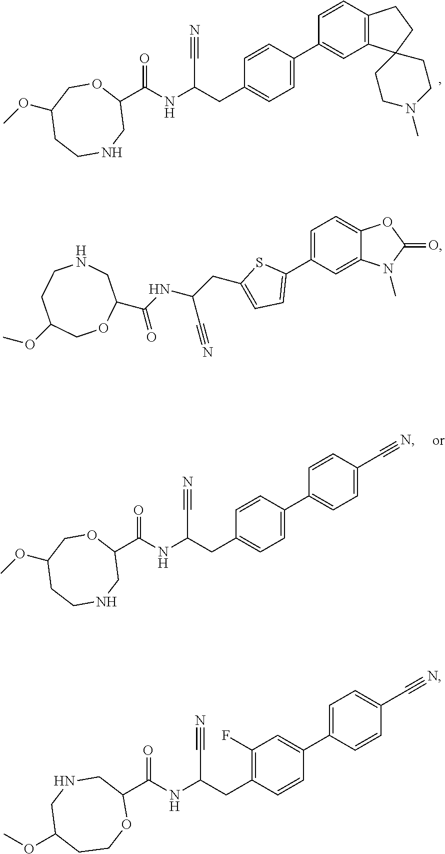 [SMILES: COC1CCNCC(C(=O)NC(C#N)Cc2ccc(-c3ccc4c(c3)C3(CC4)CCN(C)CC3)cc2)OC1 ; COC1CCNCC(C(=O)NC(C#N)Cc2ccc(-c3ccc4oc(=O)n(C)c4c3)s2)OC1 ; COC1CCNCC(C(=O)NC(C#N)Cc2ccc(-c3ccc(C#N)cc3)cc2)OC1 ; COC1CCOC(C(=O)NC(C#N)Cc2ccc(-c3ccc(C#N)cc3)cc2F)CNC1]

or a pharmaceutically acceptable salt or a stereoisomer thereof.

In embodiments of the compounds of formula (VI), or a pharmaceutically acceptable salt, a stereoisomer, or a deuterated form thereof, A is aryl or heteroaryl. In embodiments, A is a phenyl or heteroaryl. In embodiments, A is a monocyclic or a bicyclic ring.

In embodiments of the compounds of formula (VI), or a pharmaceutically acceptable salt, a stereoisomer, or a deuterated form thereof, A is a phenyl ring. In embodiments, A is

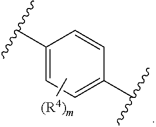

In embodiments, m is 1 or 2 and R⁴ is each independently a halogen. In embodiments, A is

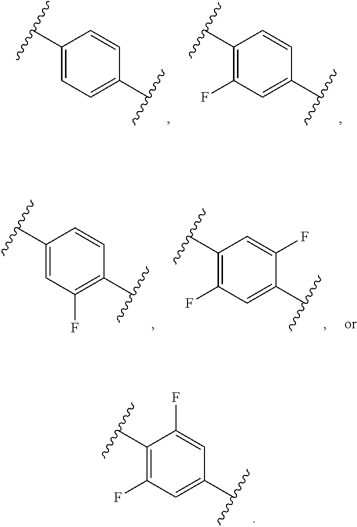 [SMILES: *c1ccc(*)cc1 ; *c1ccc(*)c(F)c1 ; *c1ccc(*)c(F)c1 ; *c1cc(F)c(*)cc1F ; *c1cc(F)c(*)c(F)c1]

In embodiments of the compounds of formula (VI), or a pharmaceutically acceptable salt, a stereoisomer, or a deuterated form thereof, A is

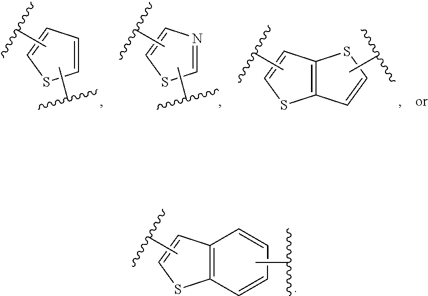

In embodiments, A is

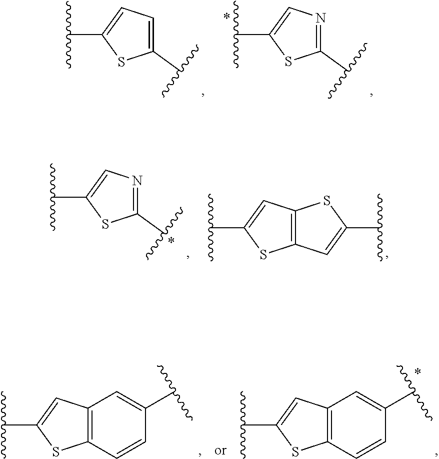 [SMILES: *c1ccc(*)s1 ; *c1cnc(*)s1 ; *c1cnc(*)s1 ; *c1cc2sc(*)cc2s1 ; *c1ccc2sc(*)cc2c1 ; *c1ccc2sc(*)cc2c1]

wherein * denotes connectivity to R². In embodiments, A is

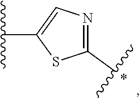 [SMILES: *c1cnc(*)s1]

wherein * denotes connectivity to R².

In embodiments of the compounds of formula (VI), or a pharmaceutically acceptable salt, a stereoisomer, or a deuterated form thereof, R¹ is

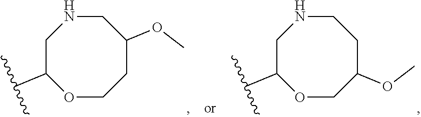 [SMILES: *C1CNCC(OC)CCO1 ; *C1CNCCC(OC)CO1]

each of which is optionally substituted with 1, 2, or 3 R⁹. In embodiments, wherein R¹ is

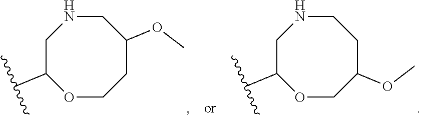 [SMILES: *C1CNCC(OC)CCO1 ; *C1CNCCC(OC)CO1]

In embodiments of the compounds of formula (VI), or a pharmaceutically acceptable salt, a stereoisomer, or a deuterated form thereof, R² is aryl, wherein R² is optionally substituted with 1, 2, 3, or 4 R⁶. In embodiments, R² is phenyl, wherein R² is optionally substituted with 1, 2, 3, or 4 R⁶. In embodiments, R² is

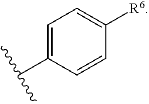

In embodiments, R² is

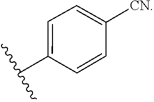 [SMILES: *c1ccc(C#N)cc1]

In embodiments, R² is

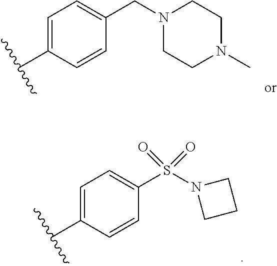 [SMILES: *c1ccc(CN2CCN(C)CC2)cc1 ; *c1ccc(S(=O)(=O)N2CCC2)cc1]

In embodiments of the compounds of formula (VI), or a pharmaceutically acceptable salt, a stereoisomer, or a deuterated form thereof, R² is a bicyclic carbocycle, bicyclic aryl, bicyclic heterocycle, or bicyclic heteroaryl, wherein R² is optionally substituted with 1, 2, 3, or 4 R⁶. In one embodiment, R² is a fused ring or a spiral ring.

In embodiments of the compounds of formula (VI), or a pharmaceutically acceptable salt, a stereoisomer, or a deuterated form thereof,
R² is

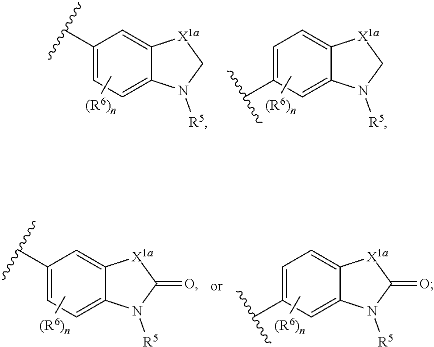

each X^{1a} is independently —NR⁵—, —O—, —CR¹¹R¹²—, —C(O)—, —S—, —S(O)— or —S(O)₂—;
each R⁵ is independently H, —C₁-C₆ alkyl, —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, or —(C₁-C₆ alkylene)-heterocycle;
each R⁶ is independently halogen, —OH, —CN, —C₁-C₆ alkyl, or —C₁-C₆ alkyl-OH;
R⁷ and R⁸ are each independently hydrogen, C₁₋₆ alkyl, or —(C₁-C₆ alkylene)-O—(C₁-C₆ alkyl);
R¹¹ and R¹² are each independently hydrogen, halogen, or —C₁-C₆ alkyl; and
n is 0, 1, 2, or 3.

In embodiments of the compounds of formula (VI), or a pharmaceutically acceptable salt, a stereoisomer, or a deuterated form thereof, R² is

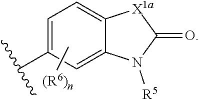

In embodiments, R⁵ is —C₁-C₆ alkyl, —CH₂CH₂OCH₂CH₃, —CH₂CH₂OCH₂CH₂CH₃, —CH₂CH₂OCH₂CH₂CH₂CH₃, —CH₂CH₂OCH₂CH₂OH, —CH₂CH₂OCH₂CH₂OCH₃, —CH₂CH₂N(CH₃)₂, or

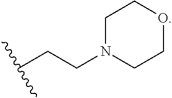 [SMILES: *CCN1CCOCC1]

In embodiments, R⁵ is —CH₂CH₂OCH₂CH₃, —CH₂CH₂OCH₂CH₂CH₃, —CH₂CH₂OCH₂CH₂CH₂CH₃, —CH₂CH₂OCH₂CH₂OH, —CH₂CH₂OCH₂CH₂OCH₃, —CH₂CH₂N(CH₃)₂, or

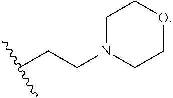 [SMILES: *CCN1CCOCC1]

In embodiments of the compounds of formula (VI), or a pharmaceutically acceptable salt, a stereoisomer, or a deuterated form thereof,
R² is

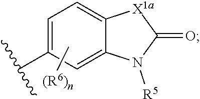

X^{1a} is —O— or —CR¹¹R¹²—;
R⁵ is —C₁-C₆ alkyl, —(C₁-C₆ alkylene)-O—(C₁-C₆ alkyl), —C₁-C₆ alkyl-NR⁷R⁸;
R⁷ and R⁸ are each independently C₁₋₆ alkyl, or —(C₁-C₆ alkylene)-O—(C₁-C₆ alkyl);
R¹¹ and R¹² are each independently —C₁-C₆ alkyl; and
n is 0.

In embodiments of the compounds of formula (VI), or a pharmaceutically acceptable salt, a stereoisomer, or a deuterated form thereof, X^{1a} is —O—.

In embodiments of the compounds of formula (VI), or a pharmaceutically acceptable salt, a stereoisomer, or a deuterated form thereof, R² is

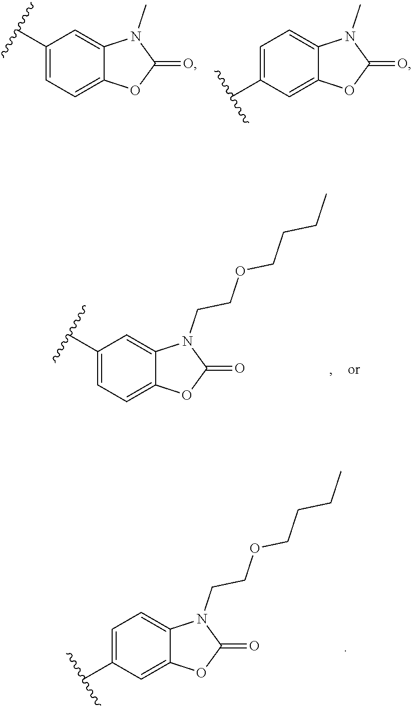 [SMILES: *c1ccc2oc(=O)n(C)c2c1 ; *c1ccc2c(c1)oc(=O)n2C ; *c1ccc2oc(=O)n(CCOCCCC)c2c1 ; *c1ccc2c(c1)oc(=O)n2CCOCCCC]

In embodiments, R² is

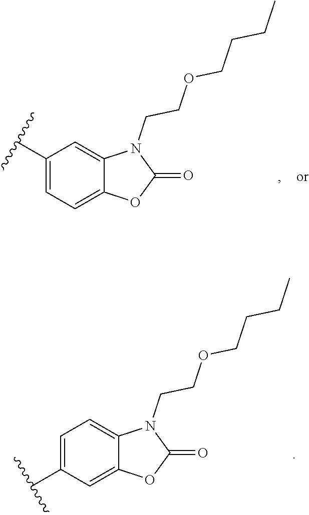 [SMILES: *c1ccc2oc(=O)n(CCOCCCC)c2c1 ; *c1ccc2c(c1)oc(=O)n2CCOCCCC]

In embodiments of the compounds of formula (VI), or a pharmaceutically acceptable salt, a stereoisomer, or a deuterated form thereof, R⁵ is —C₁-C₆ alkyl, —CH₂CH₂OCH₂CH₃, —CH₂CH₂OCH₂CH₂CH₃, —CH₂CH₂OCH₂CH₂CH₂CH₃, —CH₂CH₂OCH₂CH₂OH, —CH₂CH₂OCH₂CH₂OCH₃, —CH₂CH₂N(CH₃)₂, or

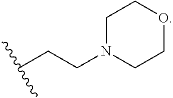 [SMILES: *CCN1CCOCC1]

In embodiments, R⁵ is —C₁-C₆ alkyl or —CH₂CH₂OCH₂CH₃.

In embodiments of the compounds of formula (VI), or a pharmaceutically acceptable salt, a stereoisomer, or a deuterated form thereof, R² is tricyclic ring, optionally substituted with 1, 2, 3, or 4 R⁵, and optionally substituted with 1, 2, 3, or 4 R⁶, wherein each R⁵ is bound to a ring nitrogen.

In embodiments of the compounds of formula (VI), or a pharmaceutically acceptable salt, a stereoisomer, or a deuterated form thereof, R² is

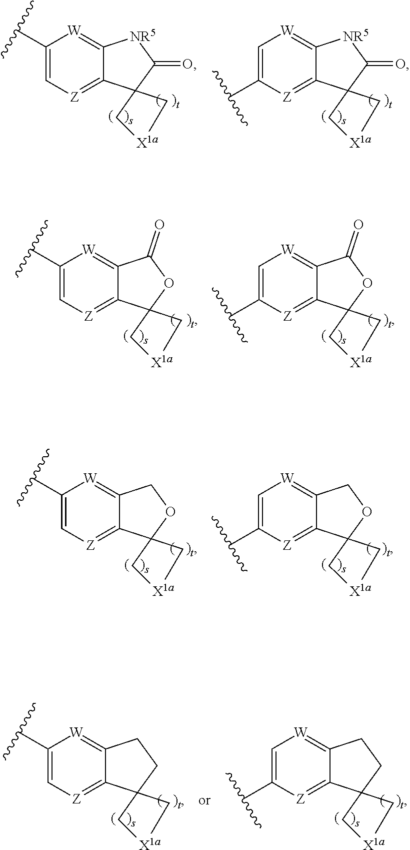

wherein R² is optionally substituted with 1 or 2 R⁶. In one embodiment, W and Z are both CH or W is CH and Z is N. In embodiments, X^{1a} is —NR⁵—. In embodiments, R⁵ is H, —C₁-C₆ alkyl, or 4-6 membered heterocycle. In embodiments, R⁵ is H, methyl or

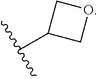 [SMILES: *C1COC1]

In embodiments, a) s is 2 and t is 2; b) s is 1 and t is 3 or s is 3 and t is 1; c) s is 1 and t is 2 or s is 2 and t is 1; or d) s is 1 and t is 1.

In embodiments of the compounds of formula (VI), or a pharmaceutically acceptable salt, a stereoisomer, or a deuterated form thereof, R² is

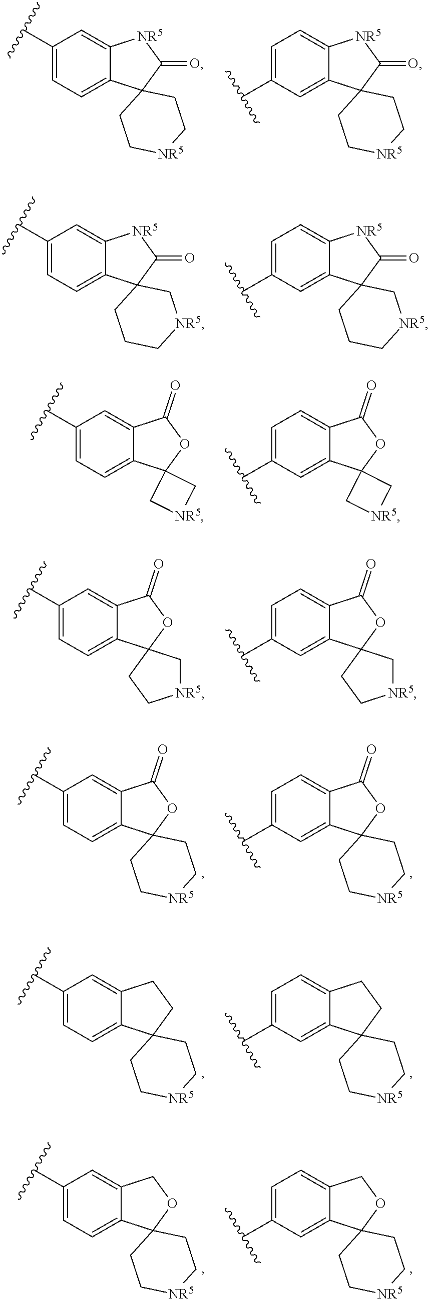

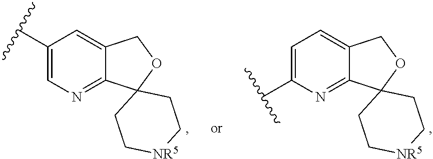

wherein each R² is optionally substituted with 1 or 2 R⁶. In embodiments, R² is

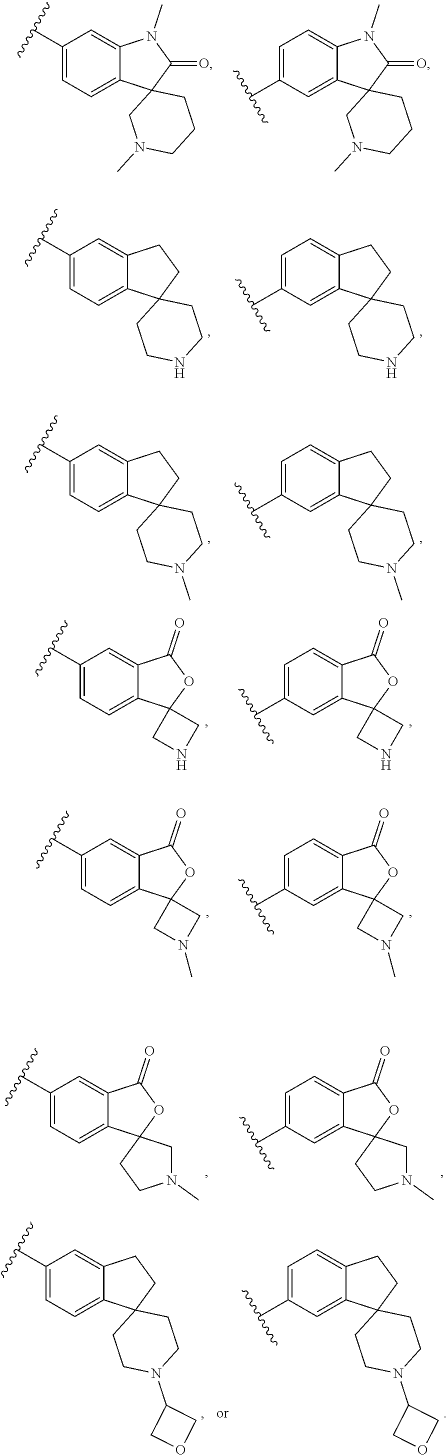

In embodiments, R² is

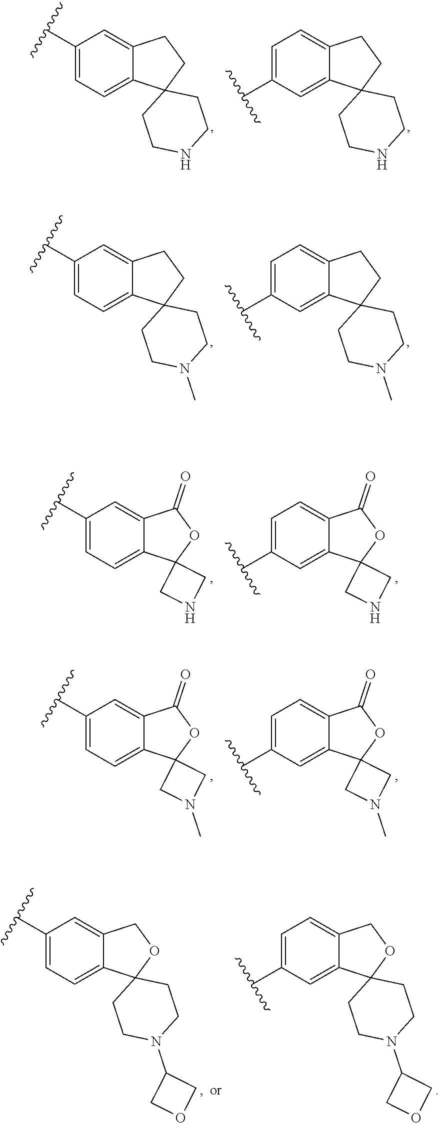

In embodiments, R² is

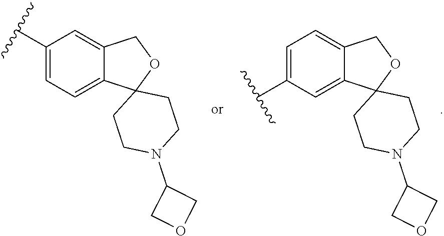 [SMILES: *c1ccc2c(c1)COC21CCN(C2COC2)CC1 ; *c1ccc2c(c1)C1(CCN(C3COC3)CC1)OC2]

In embodiments, R² is

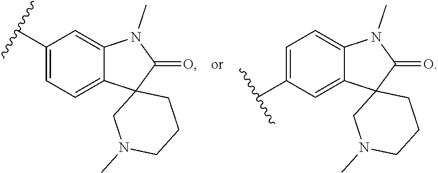 [SMILES: *c1ccc2c(c1)N(C)C(=O)C21CCCN(C)C1 ; *c1ccc2c(c1)C1(CCCN(C)C1)C(=O)N2C]

In embodiments of the compounds of formula (VI), or a pharmaceutically acceptable salt, a stereoisomer, or a deuterated form thereof, R² is

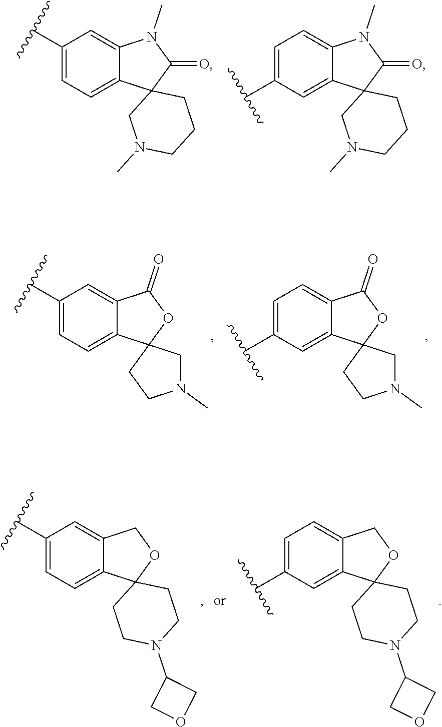 [SMILES: *c1ccc2c(c1)N(C)C(=O)C21CCCN(C)C1 ; *c1ccc2c(c1)C1(CCCN(C)C1)C(=O)N2C ; *c1ccc2c(c1)C(=O)OC21CCN(C)C1 ; *c1ccc2c(c1)C1(CCN(C)C1)OC2=O ; *c1ccc2c(c1)COC21CCN(C2COC2)CC1 ; *c1ccc2c(c1)C1(CCN(C3COC3)CC1)OC2]

In embodiments of the compounds of formula (VI), or a pharmaceutically acceptable salt, a stereoisomer, or a deuterated form thereof, R¹ is

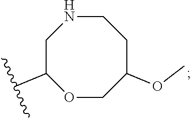 [SMILES: *C1CNCCC(OC)CO1]

### R² is

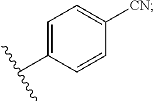 [SMILES: *c1ccc(C#N)cc1]

### and A is

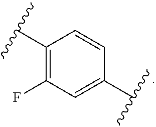 [SMILES: *c1ccc(*)c(F)c1]

In embodiments, the present disclosure provides a compound of formula (VII):

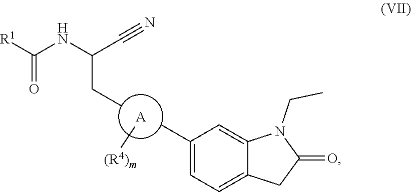

or a pharmaceutically acceptable salt, a stereoisomer, or a deuterated form thereof, wherein:
ring A is a carbocyclyl, aryl, heterocyclyl, or heteroaryl ring;
R¹ is

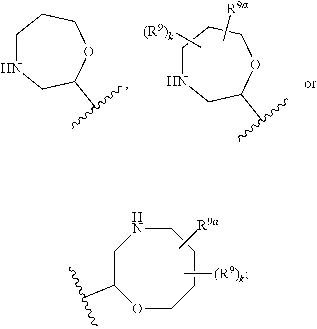

each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁹ is independently halogen, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, or —S(═NH)(O)(C₁-C₆ alkyl;
R^{9a} is —OH or —OC₁-C₃ alkyl;
m is 0, 1, or 2; and
k is 0, 1, 2, 3, or 4.

In embodiments of the compounds of formula (VII), or a pharmaceutically acceptable salt, a stereoisomer, or a deuterated form thereof, R¹ is

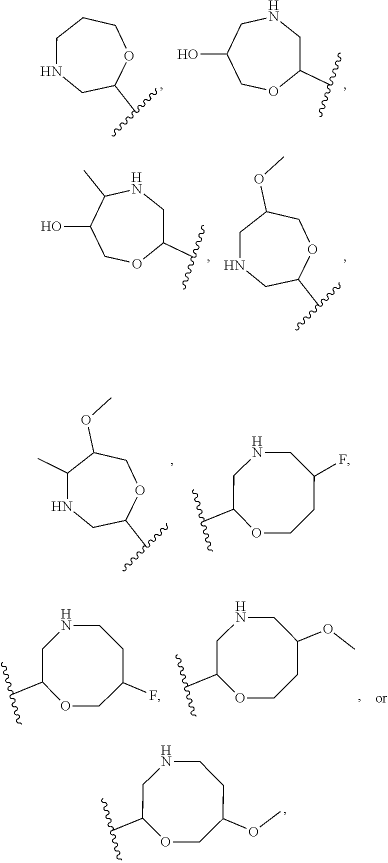

In embodiments, the present disclosure provides a compound of formula (VIII):

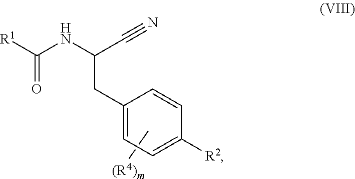

or a pharmaceutically acceptable salt, a stereoisomer, or a deuterated form thereof, wherein:
R¹ is

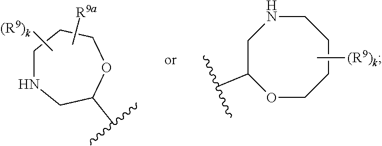

R² is

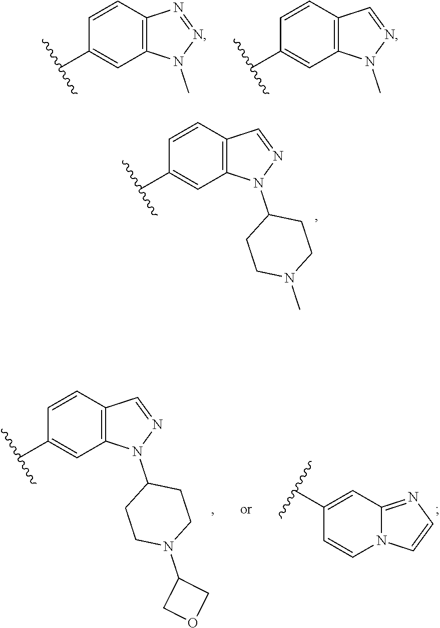

each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁹ is independently halogen, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, or —S(═NH)(O)(C₁-C₆ alkyl;
R^{9a} is —OH or —OC₁-C₃ alkyl;
m is 0, 1, or 2; and
k is 0, 1, 2, 3, or 4.

In embodiments of the compounds of formula (VIII), or a pharmaceutically acceptable salt, a stereoisomer, or a deuterated form thereof, R¹ is

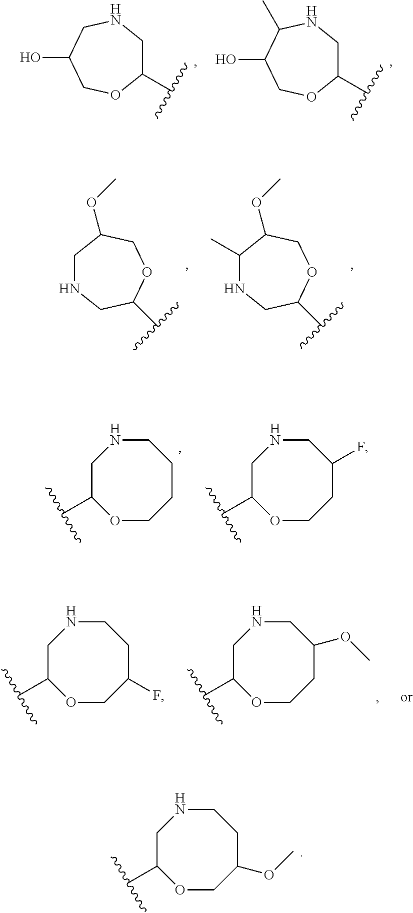

In embodiments of the compounds of formula (VII) or (VIII), or a pharmaceutically acceptable salt, a stereoisomer, or a deuterated form thereof, R^{9a} is —OH or methoxy.

In embodiments of the compounds of formula (VII) or (VIII), or a pharmaceutically acceptable salt, a stereoisomer, or a deuterated form thereof, each R⁹ is halogen, —OH, —C₁-C₆ alkyl, or —OC₁-C₆ alkyl. In embodiments, R⁹ is —C₁-C₆ alkyl. In embodiments, R⁹ is methyl.

In embodiments of the compounds of formula (VII) or (VIII), or a pharmaceutically acceptable salt, a stereoisomer, or a deuterated form thereof, m is 1 and R⁴ is halogen. In embodiments, R⁴ is F.

In embodiments of the compounds of formula (VII) or (VIII), or a pharmaceutically acceptable salt, a stereoisomer, or a deuterated form thereof, m is 0.

In embodiments of the compounds of formula (VII), or a pharmaceutically acceptable salt, a stereoisomer, or a deuterated form thereof, R¹ is

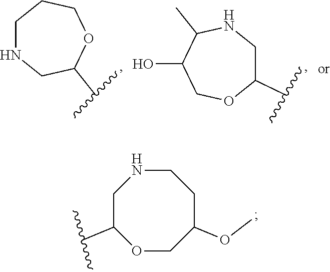

### and A is

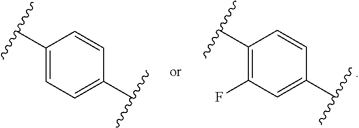 [SMILES: *c1ccc(*)cc1 ; *c1ccc(*)c(F)c1]

In embodiments of the compounds of formula (VIII), or a pharmaceutically acceptable salt, a stereoisomer, or a deuterated form thereof, R¹ is

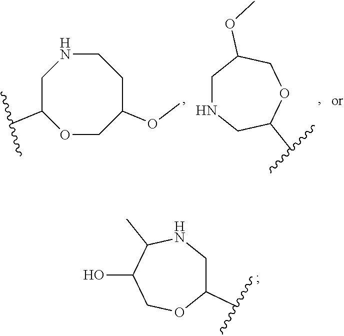

### and A is

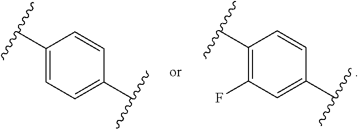 [SMILES: *c1ccc(*)cc1 ; *c1ccc(*)c(F)c1]

In embodiments, the present disclosure provides a compound of formula (IX):

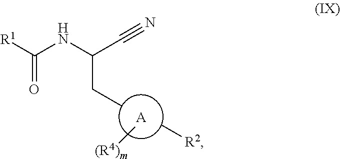

or a pharmaceutically acceptable salt, a stereoisomer, or a deuterated form thereof, wherein:
ring A is a carbocyclyl, aryl, heterocyclyl, or heteroaryl ring;
R¹ is

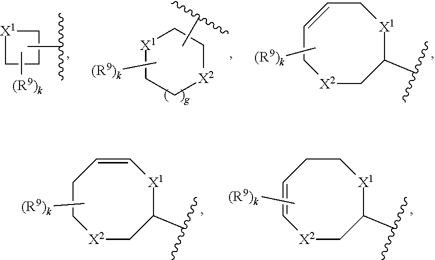

or C₁-C₆ alkylene(NH)-carbocycle substituted with 0, 1, 2, 3, or 4 R³;
X¹ is —O—, —S—, —NH—, —N(C₁-C₆ alkyl)-, —N(C₁-C₆ haloalkyl)-, —N(C(O)(C₁-C₆ alkyl))-, or —N(heterocycle)-;
X² is —O—, —S—, —NH—, —N(C₁-C₆ alkyl)-, —N(C₁-C₆ haloalkyl)-, —N(C(O)(C₁-C₆ alkyl))-, —N(heterocycle)- or —CR¹¹R¹²—;
R² is

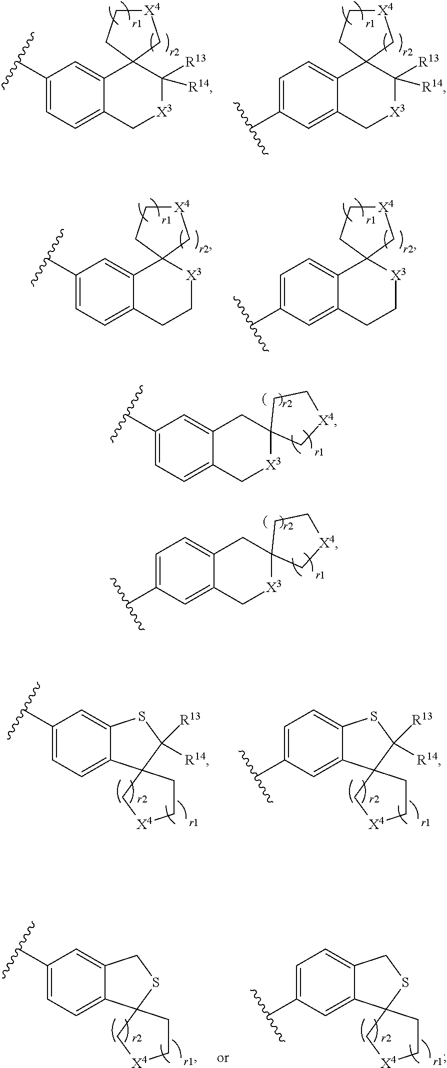

X³ is —O—, —S—, —NH—, —N(C₁-C₆ alkyl)-, —N(C₁-C₆ haloalkyl)-, —N(C(O)(C₁-C₆ alkyl))-, or —CR¹¹R¹²—;
X⁴ is —O—, —S—, —NH—, —N(CN)—, —N(C₁-C₆ alkyl)-, —N(C₁-C₆ haloalkyl)-, —N(C(O)(C₁-C₆ alkyl))-, —N(carbocycle)-, —N(heterocycle)-, or —N(heteroaryl)-, wherein each carbocycle, heterocyclyl, and heteroaryl is optionally substituted by 1 or 2 R¹⁰;
each R³ is independently halogen, —C₁-C₆ alkyl, —C₁-C₆ alkyl-OH, or —O—(C₁-C₆ alkyl);
each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁵ is independently hydrogen, halogen, —OH, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —CONR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
R¹¹ and R¹² are each independently hydrogen, halogen, or —C₁-C₆ alkyl;
R¹³ and R¹⁴ are each H or R¹³ and R¹⁴ together forms an oxo (═O);
g is 0, 1, 2, or 3;
k is 0, 1, 2, 3, or 4;
m is 0, 1, 2, or 3;
n is 0, 1, 2, 3, or 4;
r1 is 1 or 2; and
r2 is 1 or 2.

In embodiments of the compounds of formula (IX), or a pharmaceutically acceptable salt, a stereoisomer, or a deuterated form thereof, wherein:
R¹ is

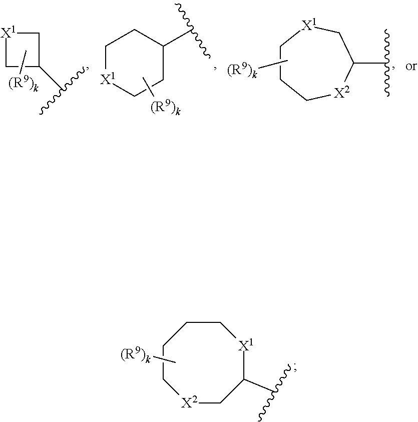

X¹ is —O—, —S—, or —NH—;
X² is —O—, —S—, —NH—, or —CH₂—; and
each R⁹ is independently —OH, fluoro, methyl, ethyl, —CH₂OH, or —OCH₃.

In embodiments of the compounds of formula (IX), or a pharmaceutically acceptable salt, a stereoisomer, or a deuterated form thereof, R¹ is

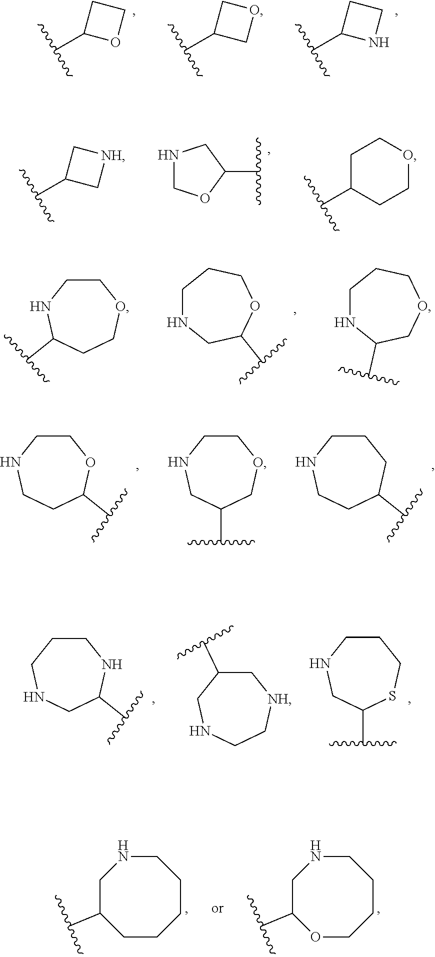

each of which is optionally substituted with 1, 2, 3, or 4 R⁹. In embodiments, R¹ is

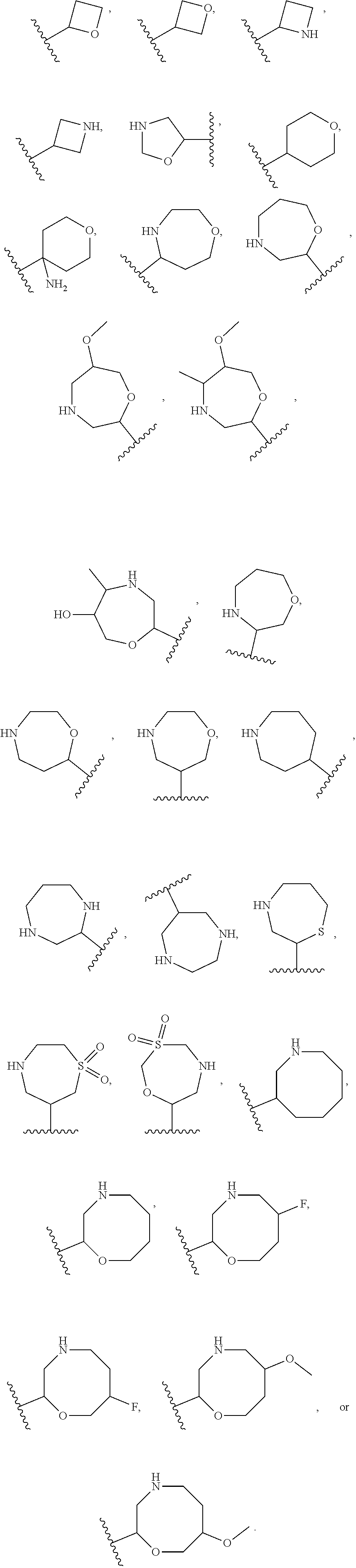

In embodiments, R¹ is

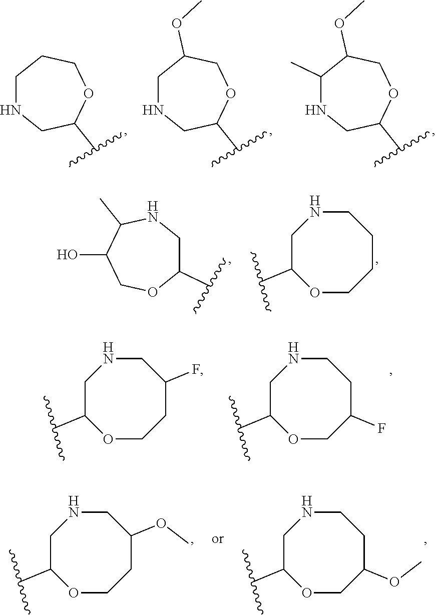

In embodiments, R¹ is

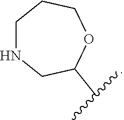 [SMILES: *C1CNCCCO1]

In embodiments of the compounds of formula (IX), or a pharmaceutically acceptable salt, a stereoisomer, or a deuterated form thereof, X³ is —O— or —S—.

In embodiments of the compounds of formula (IX), or a pharmaceutically acceptable salt, a stereoisomer, or a deuterated form thereof, X⁴ is —NH—, —N(CN)—, —N(C₁-C₆ alkyl)-, —N(C₁-C₆ haloalkyl)-, —N(carbocycle)-, —N(heterocycle)-, or —N(heteroaryl)-, wherein each carbocycle, heterocycle and heteroaryl is optionally substituted by 1 or 2 R¹⁰. In embodiments, X⁴ is —NH—, —N(CN)—, —N(C₁-C₃ alkyl)-, —N(C₁-C₃ haloalkyl)-, —N(C₃-C₆ carbocycle)-, —N(3-6 membered heterocycle)-, or —N(5- or 6-membered heteroaryl)-, wherein each carbocycle, heterocycle and heteroaryl is optionally substituted by 1 or 2 R¹⁰ selected from halogen, —OH, —C₁-C₃ alkyl, or —C₁-C₃ haloalkyl. In embodiments, X⁴ is —NH—, —N(CN)—, —N(C₁-C₃ alkyl)-, —N(C₁-C₃ haloalkyl)-, —N(C₃-C₆ carbocycle)-, —N(3-6 membered heterocycle)-, or —N(5- or 6-membered heteroaryl)-, wherein each carbocycle, heterocycle and heteroaryl is optionally substituted by 1 or 2 R¹⁰ selected from halogen, —OH, —C₁-C₃ alkyl, or —C₁-C₃ haloalkyl; wherein C₃-C₆ carbocycle is cyclopropyl, cyclobutyl, cyclopentyl, or cyclohexyl; 3-6 membered heterocycle is oxetanyl, tetrahyfuranyl, pyrrolidinyl, or piperidinyl; and 5- or 6-membered heteroaryl is thiophene or thiazole.

In embodiments of the compounds of formula (IX), or a pharmaceutically acceptable salt, a stereoisomer, or a deuterated form thereof, X⁴ is —NH—, —N(CN)—, —N(CH₃)—, —N(CH₂CH₃)—, —N(CH₂CH₂CH₃)—, —N(CH(CH₃)₂)—, —N(CF₃)—,

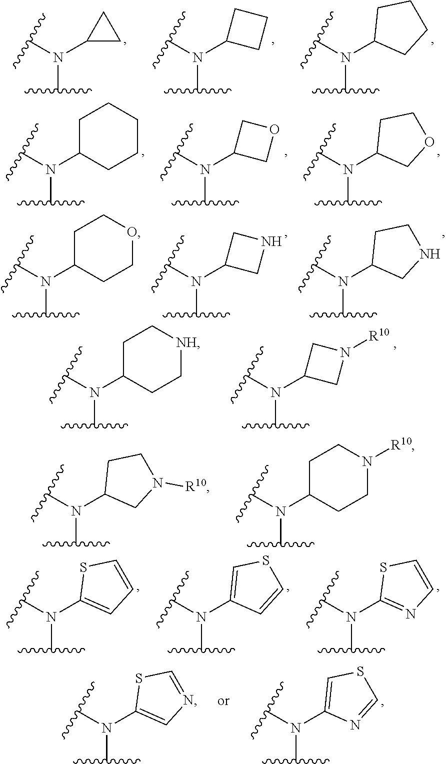

wherein R¹⁰ is —C₁-C₃ alkyl. In embodiments, X⁴ is —NH—, —N(CN)—, —N(CH₃)—, —N(CH₂CH₃)—, —N(CH₂CH₂CH₃)—, —N(CH(CH₃)₂)—, —N(CF₃)—,

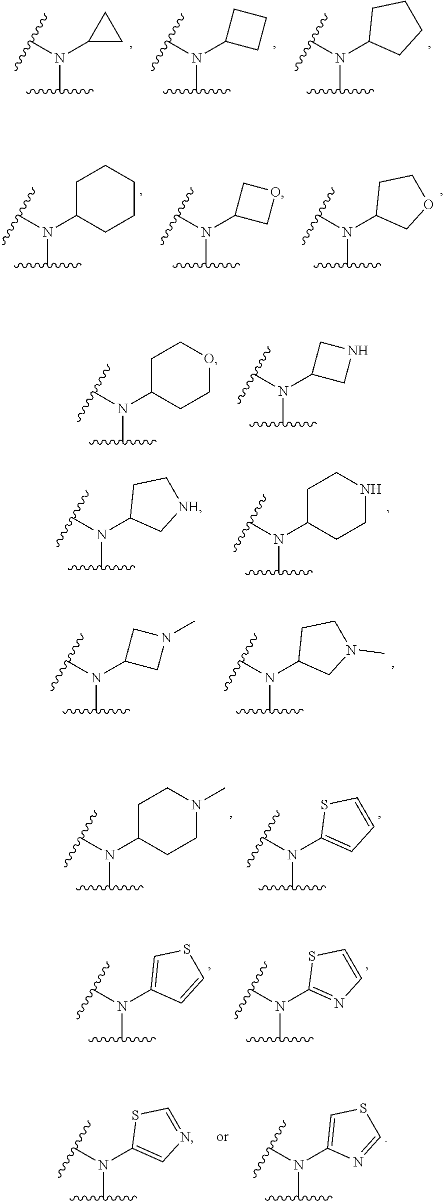

In embodiments of the compounds of formula (IX), or a pharmaceutically acceptable salt, a stereoisomer, or a deuterated form thereof, r1 is 1 and r2 is 1. In embodiments, r1 is 1 and r2 is 2. In embodiments, r1 is 2 and r2 is 1.

In embodiments of the compounds of formula (IX), or a pharmaceutically acceptable salt, a stereoisomer, or a deuterated form thereof, R² is

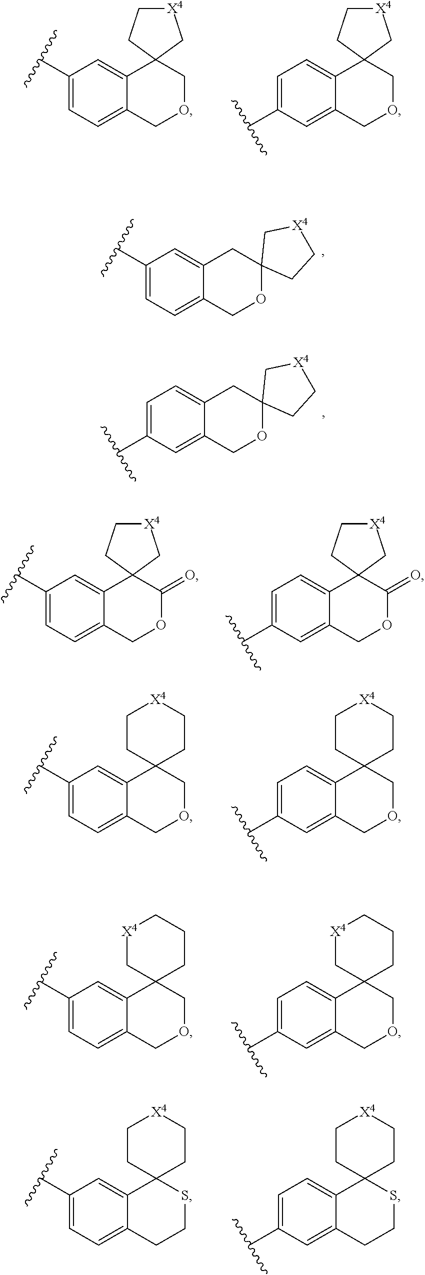

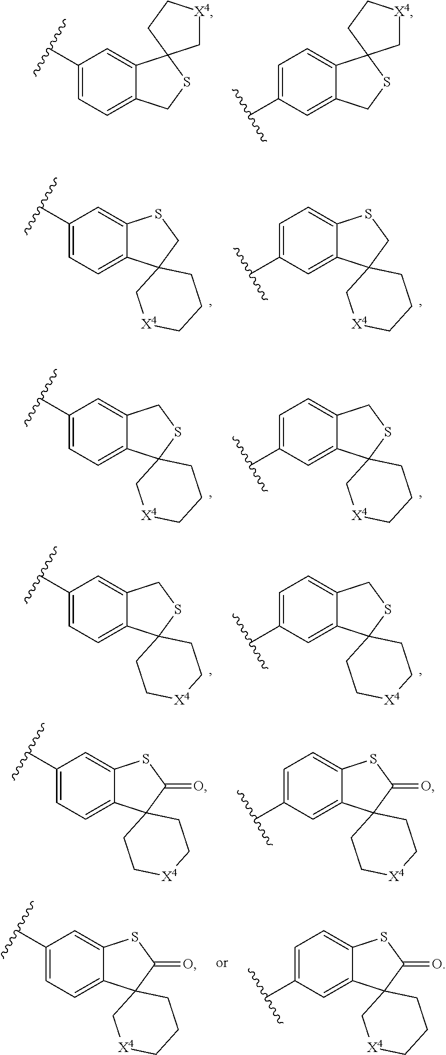

In embodiments, X⁴ is —NH—, —N(CN)—, —N(CH₃)—, —N(CH₂CH₃)—, —N(CH₂CH₂CH₃)—, —N(CH(CH₃)₂)—, —N(CF₃)—,

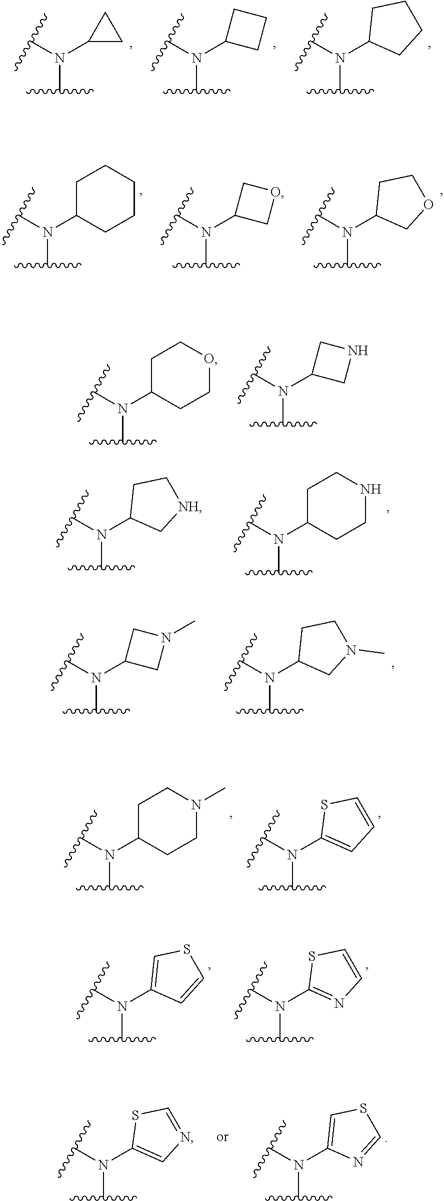

In embodiments of the compounds of formula (IX), or a pharmaceutically acceptable salt, a stereoisomer, or a deuterated form thereof, R² is

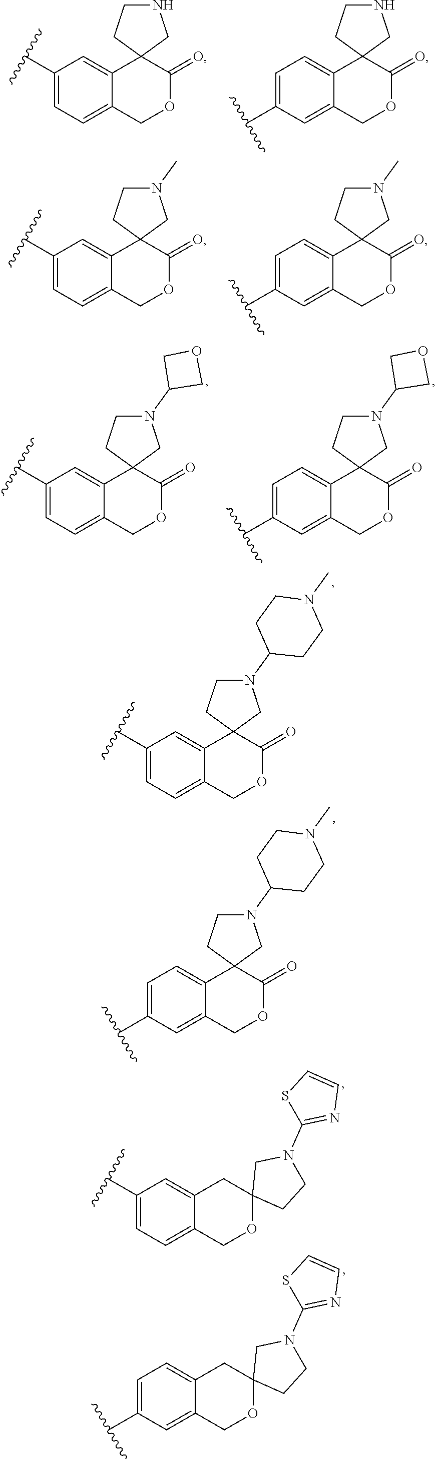

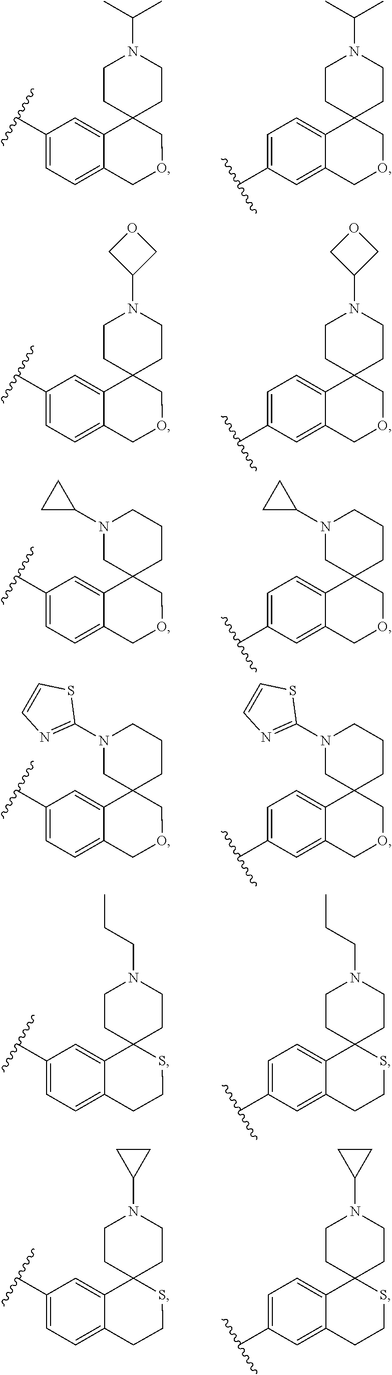

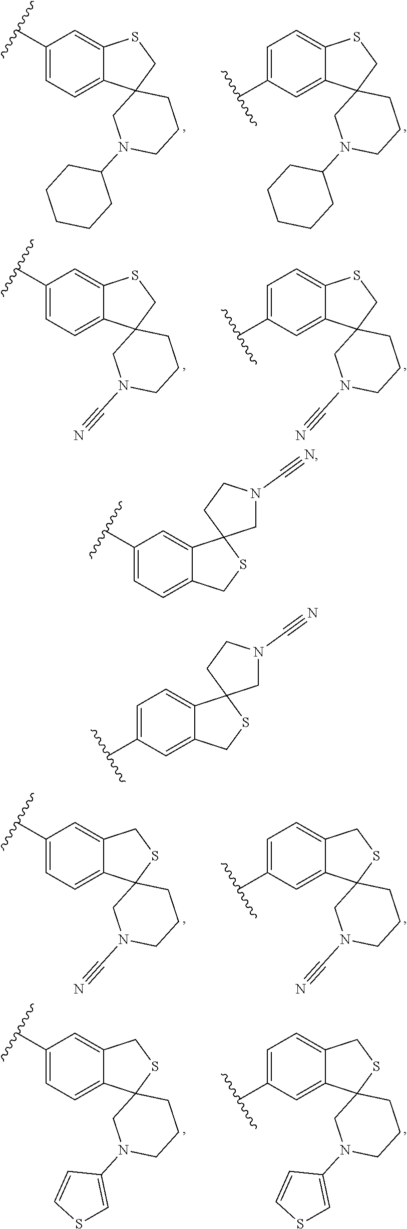

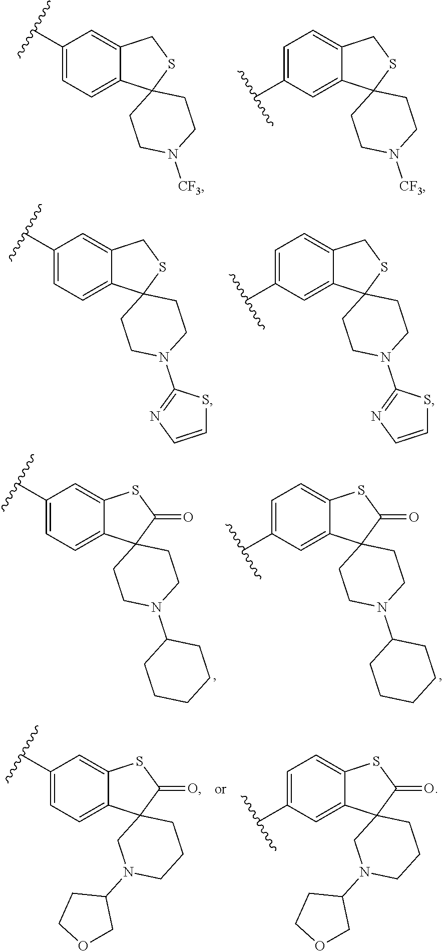

In embodiments, R² is

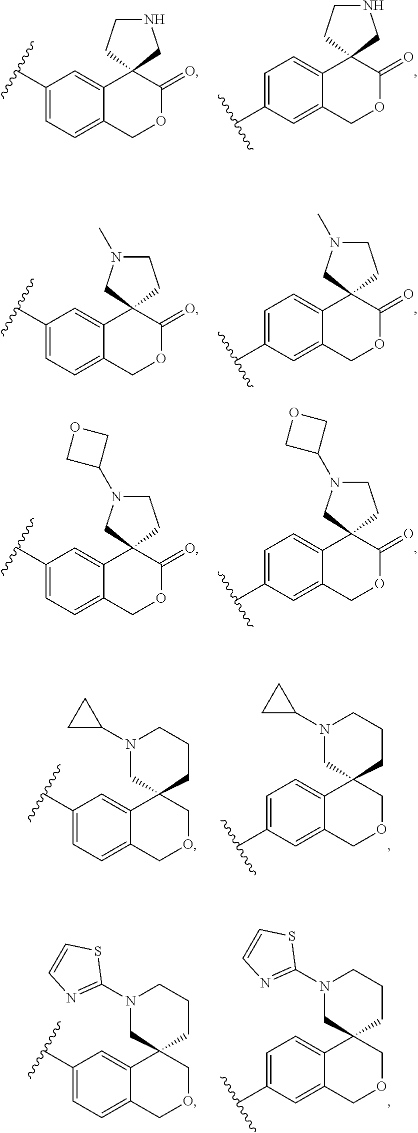

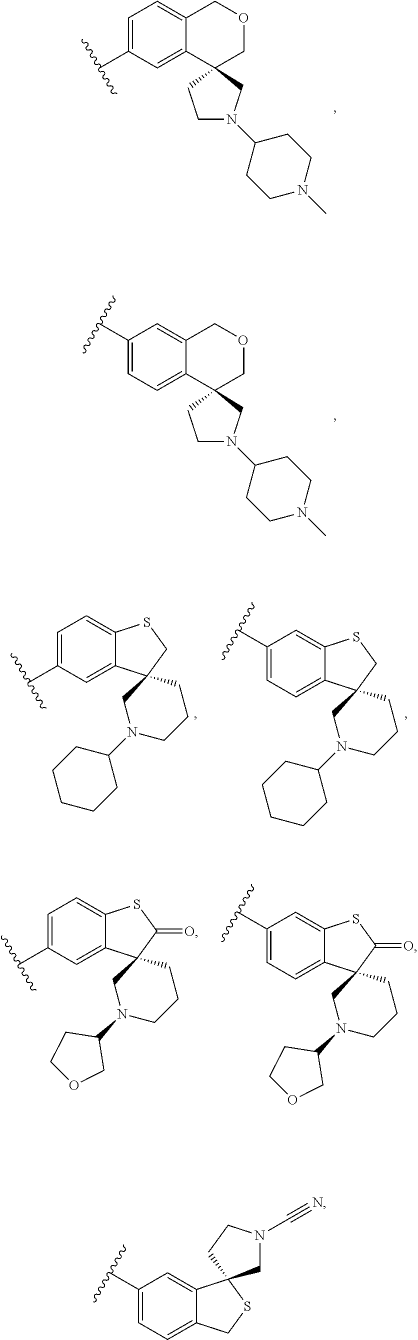

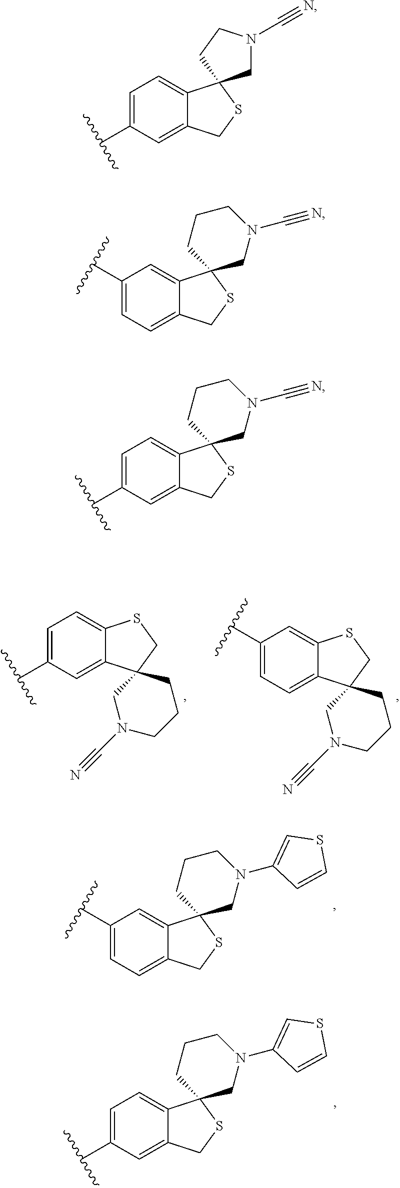

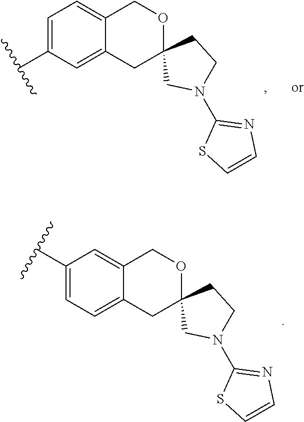 [SMILES: *c1ccc2c(c1)C[C@]1(CCN(c3nccs3)C1)OC2 ; *c1ccc2c(c1)CO[C@@]1(CCN(c3nccs3)C1)C2]

In embodiments of the compounds of formula (I)-(VI), or a pharmaceutically acceptable salt, a stereoisomer, or a deuterated form thereof, ring A is a 5-membered heteroaryl, 6-membered heteroaryl, 5,6-fused heteroaryl, 6,5-fused heteroaryl, or 6,6-fused heteroaryl. In embodiments, ring A contains one or two heteroatoms selected from N, S, or O. In embodiments, ring A is

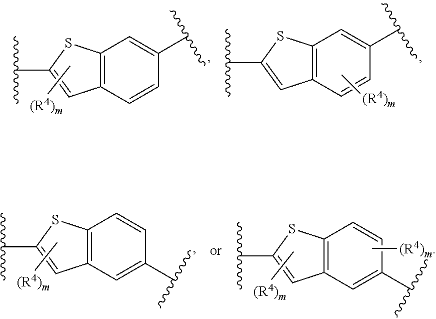

In some embodiments, ring A is

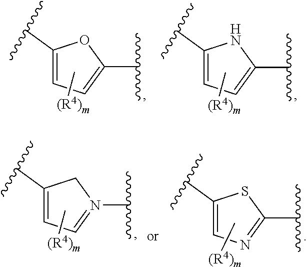

In embodiments of the compounds of formula (I), (I′), (III), (III′), (III″), (IV), (VII), (VIII) or (IX). or a pharmaceutically acceptable salt, a stereoisomer, or a deuterated form thereof, R¹ is

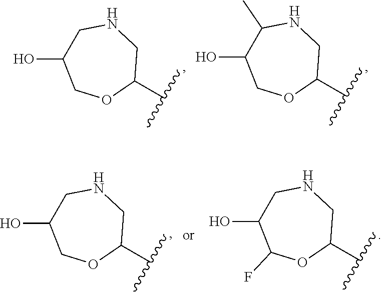

In embodiments, R¹ is

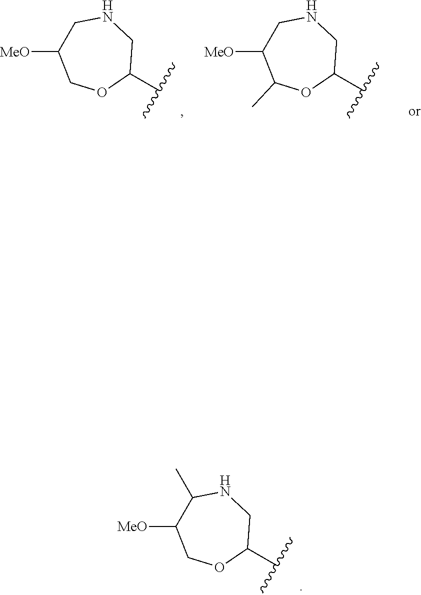 [SMILES: *C1CNCC(OC)CO1 ; *C1CNCC(OC)C(C)O1 ; *C1CNC(C)C(OC)CO1]

In embodiments, R¹ is

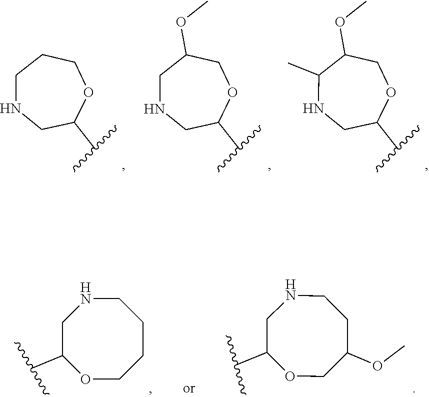

In embodiments, R¹ is

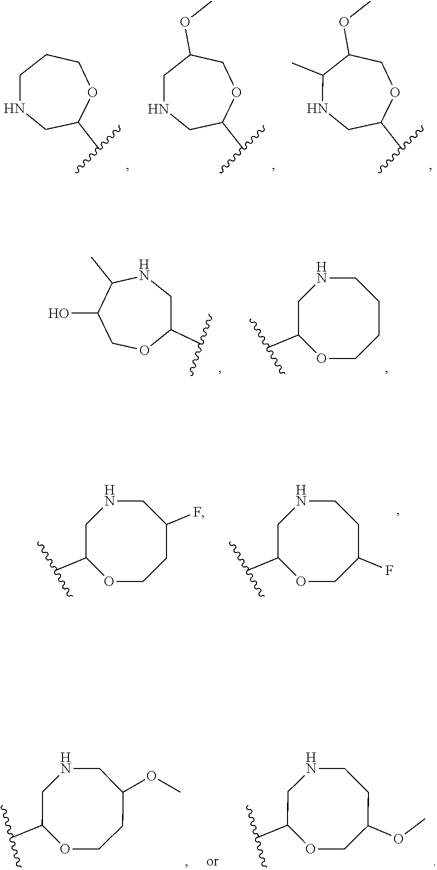

In embodiments of the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or a pharmaceutically acceptable salt, a stereoisomer, or a deuterated form thereof, at least one R⁴ is a halogen.

In embodiments of the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or a pharmaceutically acceptable salt, a stereoisomer, or a deuterated form thereof, each R⁴ is independently halogen, —C₁-C₃ alkyl, or —CN.

In embodiments of the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or a pharmaceutically acceptable salt, a stereoisomer, or a deuterated form thereof, R⁴ is halogen and R⁶ is —S(C₁-C₆ alkyl), —SO(C₁-C₆ alkyl), —SO₂(C₁-C₆ alkyl), —SO₂N(C₁-C₆ alkyl), or —SO₂NR⁷R⁸, wherein R⁷ and R⁸ are each independently hydrogen or C₁₋₄ alkyl.

In embodiments of the compounds of formula (I), (I′), (I-A), (I-B), (I-C), (I-D), (I-E), (II), (II-A), (II-B) (III), (III′), (III″), (III-A), (V), (VI), or (IX) or a pharmaceutically acceptable salt, a stereoisomer, or a deuterated form thereof, R⁵ is hydrogen, —C₁-C₆ alkyl, or —CONR⁷R⁸, wherein R⁷ and R⁸ are each independently hydrogen or C₁₋₄ alkyl. In embodiments, R⁵ is hydrogen, methyl, or —CONH₂.

In embodiments of the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or a pharmaceutically acceptable salt, a stereoisomer, or a deuterated form thereof, m is 0 or 1. In some embodiments, m is 0. In some embodiments, m is 1.

In embodiments of the compounds of formula (I), (I′), (II), (IV)-(VI), or (IX) or a pharmaceutically acceptable salt, a stereoisomer, or a deuterated form thereof, each R⁶ is independently halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂NH(C₁-C₃ alkyl), —SO₂NR⁷R⁸, or 4-6 membered heterocycle. In some embodiments, R⁶ is independently halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂NH₂, —SO₂NH(C₁-C₃ alkyl), —SO₂N(C₁-C₃ alkyl)₂, 4-6 membered heterocycle, or —(C₁-C₆ alkylene)-(4-6 membered heterocycle). R⁶ is independently halogen, —OH, —CN, —SO₂CH₃,

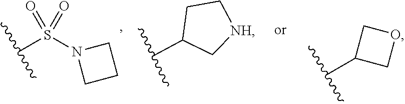

In some embodiments, R⁶ is independently halogen, —OH, —CN, —SO₂CH₃,

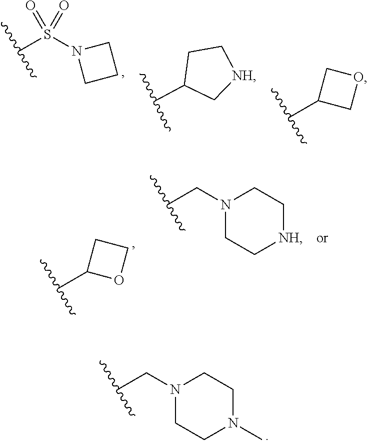

In embodiments of the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or a pharmaceutically acceptable salt, a stereoisomer, or a deuterated form thereof, wherein R⁷ and R⁸ are each independently hydrogen or C₁₋₄ alkyl.

In embodiments of the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or a pharmaceutically acceptable salt, a stereoisomer, or a deuterated form thereof, R⁹ is halogen, —C₁-C₃ alkyl, —OH, —OCH₃, —OCH₂CH₃, —OCD₃, —OCF₃, or —OCHF₂. In embodiments, R⁹ is —OH, —OCH₃, or methyl.

In embodiments of the compounds of formula (I), (I′), (II), (III), (III′), (III″), (III-A), (IV), (V), (V-A), (V-B), (VI), or (IX), or a pharmaceutically acceptable salt, a stereoisomer, or a deuterated form thereof, R¹⁰ is —C₁-C₆ alkyl, —C₁-C₆ alkyl-OH, —C₁-C₁₀ haloalkyl, —C₁-C₁₀ haloalkyl-OH, —(C₁-C₃ alkylene)-O—(C₁-C₄ alkyl), —(C₁-C₃ alkylene)-O—(C₁-C₄ alkyl)-OH, —(C₁-C₃ alkylene)-O—(C₁-C₃ alkylene)-O—(C₁-C₄ alkyl), —(C₁-C₃ alkylene)-O—(C₁-C₃ alkylene)-O—(C₁-C₄ alkyl)-OH, —C₁-C₃ alkyl-NR⁷R⁸, or —(C₁-C₃ alkylene)-heterocycle. In some embodiments, R¹⁰ is —C₁-C₆ alkyl, —(C₁-C₃ alkylene)-O—(C₁-C₄ alkyl), —(C₁-C₃ alkylene)-O—(C₁-C₃ alkyl)-OH, —(C₁-C₃ alkylene)-O—(C₁-C₃ alkylene)-O—(C₁-C₃ alkyl), —(C₁-C₃ alkylene)-O—(C₁-C₃ alkylene)-O—(C₁-C₃ alkyl)-OH, —C₁-C₃ alkyl-N(C₁-C₃ alkyl)₂, or —(C₁-C₃ alkylene)-(6 membered heterocyclyl).

In embodiments of the compounds of formula (I), (I′), (II), (III), (III′), (III″), (III-A), (IV), (V), (V-A), (V-B), (VI), or (IX), or a pharmaceutically acceptable salt, a stereoisomer, or a deuterated form thereof, R¹⁰ is —C₁-C₆ alkyl, —C₁-C₆ alkyl-OH, —C₁-C₁₀ haloalkyl, —C₁-C₁₀ haloalkyl-OH, —(C₁-C₃ alkylene)-O—(C₁-C₄ alkyl), —(C₁-C₃ alkylene)-O—(C₁-C₄ alkyl)-OH, —(C₁-C₃ alkylene)-O—(C₁-C₃ alkylene)-O—(C₁-C₄ alkyl), —(C₁-C₃ alkylene)-O—(C₁-C₃ alkylene)-O—(C₁-C₄ alkyl)-OH, —C₁-C₃ alkyl-NR⁷R⁸, or —(C₁-C₃ alkylene)-heterocycle. In some embodiments, R¹⁰ is —C₁-C₆ alkyl, —(C₁-C₃ alkylene)-O—(C₁-C₄ alkyl), —(C₁-C₃ alkylene)-O—(C₁-C₃ alkyl)-OH, —(C₁-C₃ alkylene)-O—(C₁-C₃ alkylene)-O—(C₁-C₃ alkyl), —(C₁-C₃ alkylene)-O—(C₁-C₃ alkylene)-O—(C₁-C₃ alkyl)-OH, —C₁-C₃ alkyl-N(C₁-C₃ alkyl)₂, or —(C₁-C₃ alkylene)-(6 membered heterocyclyl). In embodiments, R¹⁰ is —C₁-C₆ alkyl, —CH₂CH₂OCH₂CH₃, —CH₂CH₂OCH₂CH₂CH₃, —CH₂CH₂OCH₂CH₂CH₂CH₃, —CH₂CH₂OCH₂CH₂OH, —CH₂CH₂OCH₂CH₂OCH₃, —CH₂CH₂N(CH₃)₂, or

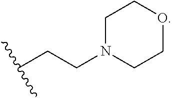 [SMILES: *CCN1CCOCC1]

In embodiments, R¹⁰ is —C₁-C₃ alkyl.

In embodiments of the compounds of formula (I), (I′), (II), (III), (III′), (III″), (III-A), (IV), or (IX) or a pharmaceutically acceptable salt, a stereoisomer, or a deuterated form thereof, R¹¹ and R¹² are each independently hydrogen, halogen, or —C₁-C₄ alkyl.

Another embodiment is a product obtainable by any of the processes or examples disclosed herein.

In embodiments, provided herein is a compound of formula (I), (I′), (II), (III), (III′), (III″), (IV), (V), (V-A), (V-B), (VI), or (IX), or a stereoisomer thereof, or a pharmaceutically acceptable salt thereof, including compounds of formula (I-A), (I-B), (I-C), (I-D), (I-E), (II-A), (II-B), (III-A), (V-A), and (V-B). When refereeing to compounds of formula (I), (I′), (II), (III), (III′), (III″), (IV), (V), (V-A), (V-B), (VI), or (IX), (or (I)-(IX)), compounds of formula (I-A), (I-B), (I-C), (I-D), (I-E), (II-A), (II-B), (III-A), (V-A), and (V-B) are encompassed unless explicitly excluded.

In embodiments, provided herein is a pharmaceutically acceptable salt of a compound of formula (I)-(IX). Further embodiments of the disclosure relate to a deuterated compound of formula (I)-(IX), or a pharmaceutically acceptable salt thereof.

In embodiments of the compound of formula (I)-(IX), or a pharmaceutically acceptable salt, a stereoisomer, or a deuterated form thereof, the compound is not a compound disclosed in WO2024/148308.

In embodiments, provided herein is a compound of formula (I) selected from Compounds A4-A52, or a pharmaceutically acceptable salt thereof, a deuterated form thereof, racemic form thereof, or stereoisomer thereof. In embodiments, the compound of formula (I) is selected from Compounds A4-A13, A15-A141, or A145-A172, or a pharmaceutically acceptable salt thereof, a deuterated form thereof, racemic form thereof, or stereoisomer thereof.

In embodiments, provided herein is a compound in Table A, or a pharmaceutically acceptable salt thereof, deuterated form thereof, racemic form thereof, or stereoisomer thereof.

In embodiments, provided herein is a compound in Table A, or a pharmaceutically acceptable salt thereof, or stereoisomer thereof.

In embodiments, provided herein is a compound in Table A, or a pharmaceutically acceptable salt thereof.

In one embodiment, provided herein is a compound set forth in Table A.

In embodiments, provided herein is a compound selected from Compounds A1-A3, or a pharmaceutically acceptable salt thereof, a deuterated form thereof, racemic form thereof, or stereoisomer thereof. In embodiments, the compound is selected from Compounds A1-A3, A14, or A142-A144, or a pharmaceutically acceptable salt thereof, a deuterated form thereof, racemic form thereof, or stereoisomer thereof.

In embodiments, the compound provided herein is Compound A8, or a pharmaceutically acceptable salt, a stereoisomer, or a deuterated form thereof.

In embodiments, the compound provided herein is Compound A10, or a pharmaceutically acceptable salt, a stereoisomer, or a deuterated form thereof.

In embodiments, the compound provided herein is Compound A27, or a pharmaceutically acceptable salt, a stereoisomer, or a deuterated form thereof.

In embodiments, the compound provided herein is Compound A28, or a pharmaceutically acceptable salt, a stereoisomer, or a deuterated form thereof.

In embodiments, the compound provided herein is Compound A38, or a pharmaceutically acceptable salt, a stereoisomer, or a deuterated form thereof.

In embodiments, the compound provided herein is Compound A43, or a pharmaceutically acceptable salt, a stereoisomer, or a deuterated form thereof.

In embodiments, the compound provided herein is Compound A47, or a pharmaceutically acceptable salt, a stereoisomer, or a deuterated form thereof.

In embodiments, the compound provided herein is Compound A88, or a pharmaceutically acceptable salt, a stereoisomer, or a deuterated form thereof.

In embodiments, the compound provided herein is Compound A92, or a pharmaceutically acceptable salt, a stereoisomer, or a deuterated form thereof.

In embodiments, the compound provided herein is Compound A105, or a pharmaceutically acceptable salt, a stereoisomer, or a deuterated form thereof.

In embodiments, the compound provided herein is Compound A109, or a pharmaceutically acceptable salt, a stereoisomer, or a deuterated form thereof.

In embodiments, the compound provided herein is Compound A111, or a pharmaceutically acceptable salt, a stereoisomer, or a deuterated form thereof.

In embodiments, the compound provided herein is Compound A117, or a pharmaceutically acceptable salt, a stereoisomer, or a deuterated form thereof.

In embodiments, the compound provided herein is Compound A164, or a pharmaceutically acceptable salt, a stereoisomer, or a deuterated form thereof.

In some embodiments, provided herein is a pharmaceutically acceptable salt of a compound in Table A.

TABLE A
Various compounds of the invention

| Compound | |
|---|---|
| No. | Structure |
| A1 | 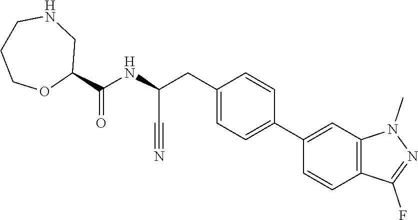 [SMILES: Cn1nc(F)c2ccc(-c3ccc(C[C@@H](C#N)NC(=O)[C@@H]4CNCCCO4)cc3)cc21] |
| A2 | 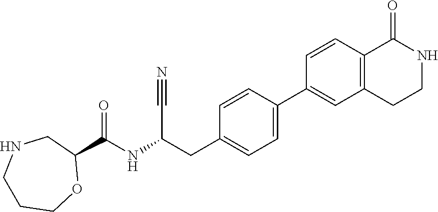 [SMILES: N#C[C@H](Cc1ccc(-c2ccc3c(c2)CCNC3=O)cc1)NC(=O)[C@@H]1CNCCCO1] |
| A3 | 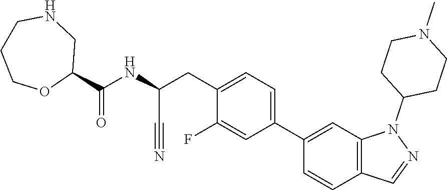 [SMILES: CN1CCC(n2ncc3ccc(-c4ccc(C[C@@H](C#N)NC(=O)[C@@H]5CNCCCO5)c(F)c4)cc32)CC1] |
| A4 | 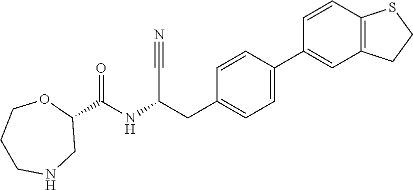 [SMILES: N#C[C@H](Cc1ccc(-c2ccc3c(c2)CCS3)cc1)NC(=O)[C@@H]1CNCCCO1] |
| A5 | 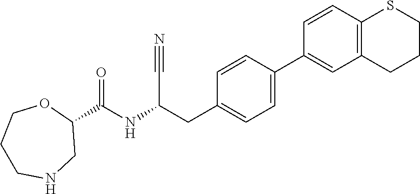 [SMILES: N#C[C@H](Cc1ccc(-c2ccc3c(c2)CCCS3)cc1)NC(=O)[C@@H]1CNCCCO1] |
| A6 | 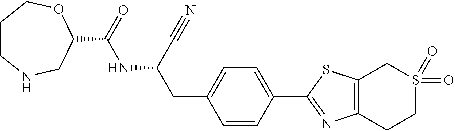 |
| A7 | 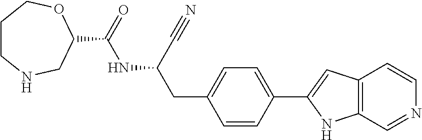 |
| A8 | 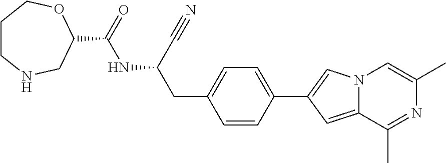 |
| A9 | 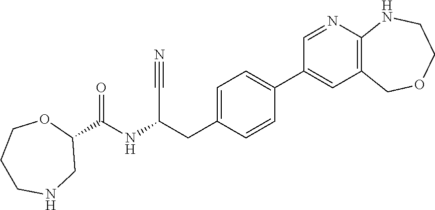 [SMILES: N#C[C@H](Cc1ccc(-c2cnc3c(c2)COCCN3)cc1)NC(=O)[C@@H]1CNCCCO1] |
| A10 | 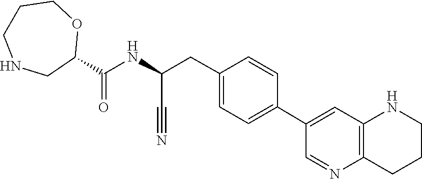 [SMILES: N#C[C@H](Cc1ccc(-c2cnc3c(c2)NCCC3)cc1)NC(=O)[C@@H]1CNCCCO1] |
| A11 | 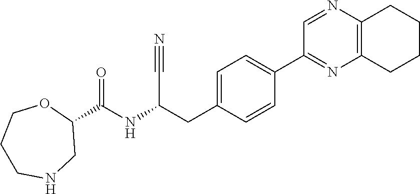 [SMILES: N#C[C@H](Cc1ccc(-c2cnc3c(n2)CCCC3)cc1)NC(=O)[C@@H]1CNCCCO1] |
| A12 | 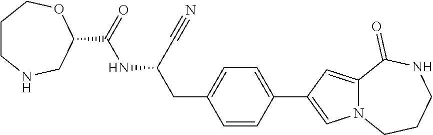 [SMILES: N#C[C@H](Cc1ccc(-c2cc3n(c2)CCCNC3=O)cc1)NC(=O)[C@@H]1CNCCCO1] |
| A13 | 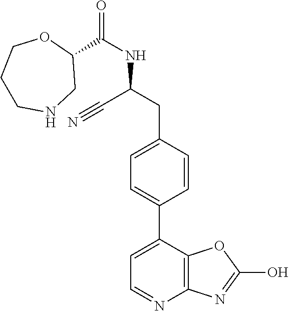 [SMILES: N#C[C@H](Cc1ccc(-c2ccnc3nc(O)oc23)cc1)NC(=O)[C@@H]1CNCCCO1] |
| A14 | 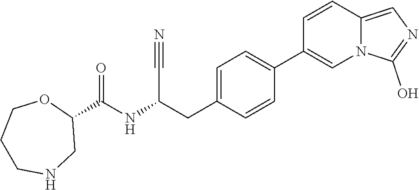 [SMILES: N#C[C@H](Cc1ccc(-c2ccc3cnc(O)n3c2)cc1)NC(=O)[C@@H]1CNCCCO1] |
| A15 | 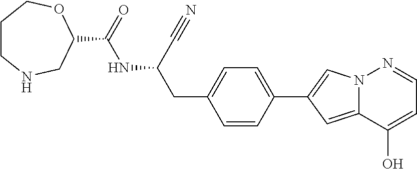 |
| A16 | 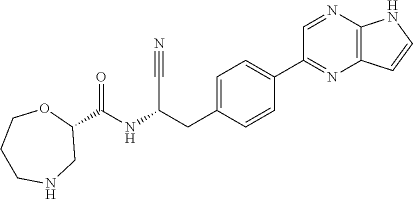 [SMILES: N#C[C@H](Cc1ccc(-c2cnc3[nH]ccc3n2)cc1)NC(=O)[C@@H]1CNCCCO1] |
| A17 | 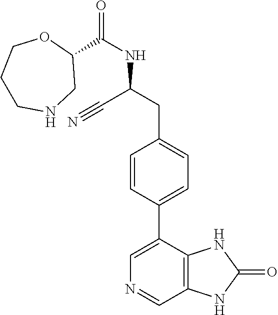 [SMILES: N#C[C@H](Cc1ccc(-c2cncc3[nH]c(=O)[nH]c23)cc1)NC(=O)[C@@H]1CNCCCO1] |
| A18 | 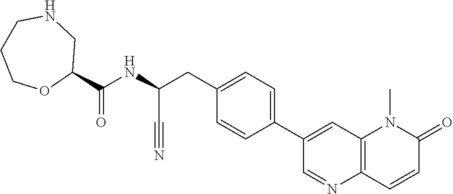 [SMILES: Cn1c(=O)ccc2ncc(-c3ccc(C[C@@H](C#N)NC(=O)[C@@H]4CNCCCO4)cc3)cc21] |
| A19 | 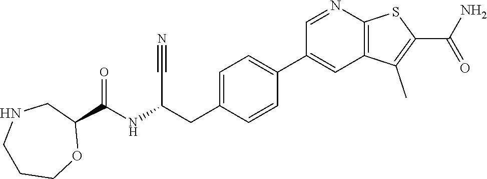 [SMILES: Cc1c(C(N)=O)sc2ncc(-c3ccc(C[C@@H](C#N)NC(=O)[C@@H]4CNCCCO4)cc3)cc12] |
| A20 | 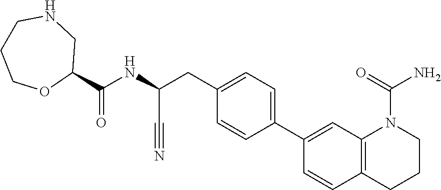 [SMILES: N#C[C@H](Cc1ccc(-c2ccc3c(c2)N(C(N)=O)CCC3)cc1)NC(=O)[C@@H]1CNCCCO1] |
| A21 | 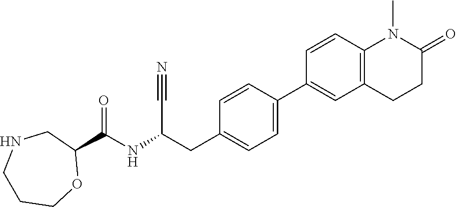 [SMILES: CN1C(=O)CCc2cc(-c3ccc(C[C@@H](C#N)NC(=O)[C@@H]4CNCCCO4)cc3)ccc21] |
| A22 | 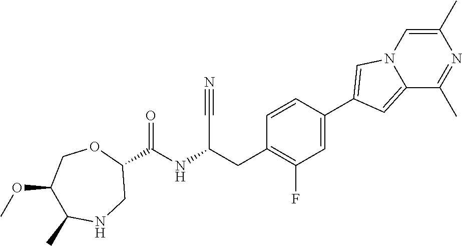 |
| A23 | 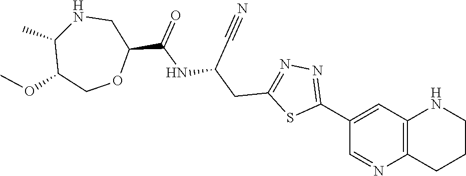 [SMILES: CO[C@H]1CO[C@H](C(=O)N[C@H](C#N)Cc2nnc(-c3cnc4c(c3)NCCC4)s2)CN[C@H]1C] |
| A24 | 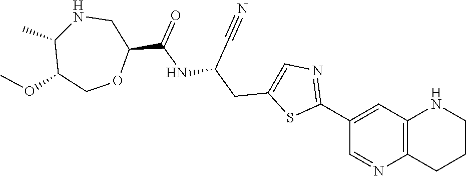 [SMILES: CO[C@H]1CO[C@H](C(=O)N[C@H](C#N)Cc2cnc(-c3cnc4c(c3)NCCC4)s2)CN[C@H]1C] |
| A25 | 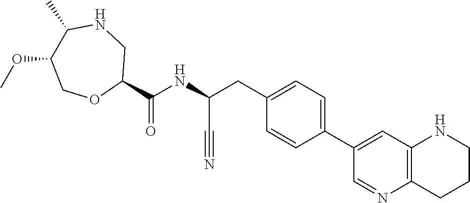 [SMILES: CO[C@H]1CO[C@H](C(=O)N[C@H](C#N)Cc2ccc(-c3cnc4c(c3)NCCC4)cc2)CN[C@H]1C] |
| A26 | 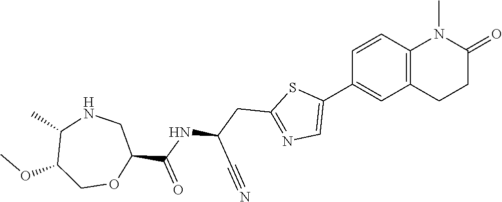 [SMILES: CO[C@H]1CO[C@H](C(=O)N[C@H](C#N)Cc2ncc(-c3ccc4c(c3)CCC(=O)N4C)s2)CN[C@H]1C] |
| A27 | 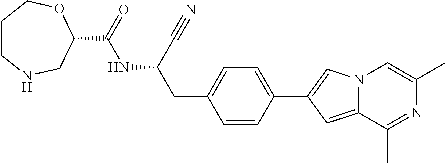 [SMILES: Cc1cn2cc(-c3ccc(C[C@@H](C#N)NC(=O)[C@@H]4CNCCCO4)cc3)cc2c(C)n1] |
| A28 | 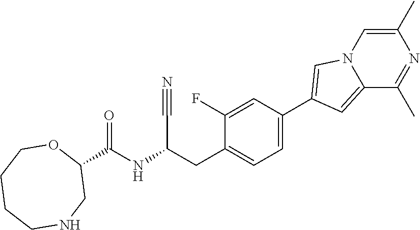 [SMILES: Cc1cn2cc(-c3ccc(C[C@@H](C#N)NC(=O)[C@@H]4CNCCCCO4)c(F)c3)cc2c(C)n1] |
| A29 | 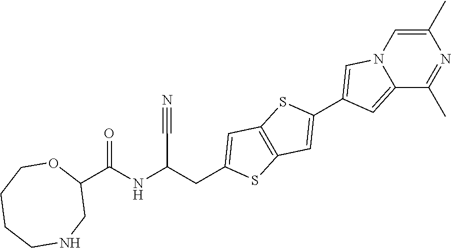 [SMILES: Cc1cn2cc(-c3cc4sc(CC(C#N)NC(=O)C5CNCCCCO5)cc4s3)cc2c(C)n1] |
| A30* | 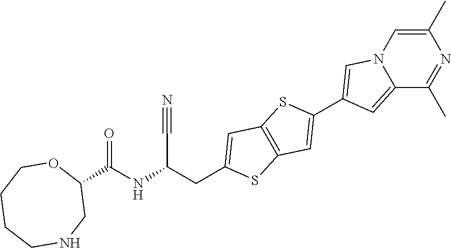 [SMILES: Cc1cn2cc(-c3cc4sc(C[C@@H](C#N)NC(=O)[C@@H]5CNCCCCO5)cc4s3)cc2c(C)n1] |
| A31* | 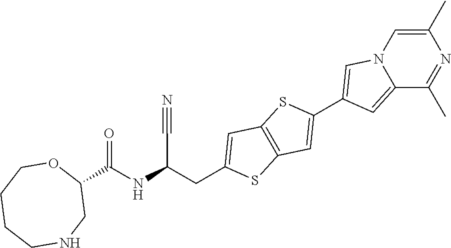 [SMILES: Cc1cn2cc(-c3cc4sc(C[C@H](C#N)NC(=O)[C@@H]5CNCCCCO5)cc4s3)cc2c(C)n1] |
| A32 | 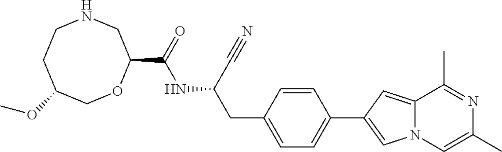 |
| A33 | 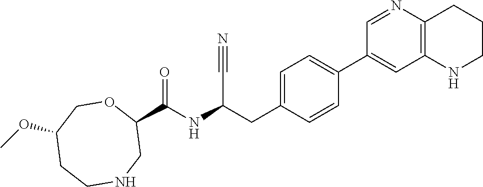 [SMILES: CO[C@H]1CCNC[C@H](C(=O)N[C@@H](C#N)Cc2ccc(-c3cnc4c(c3)NCCC4)cc2)OC1] |
| A34 | 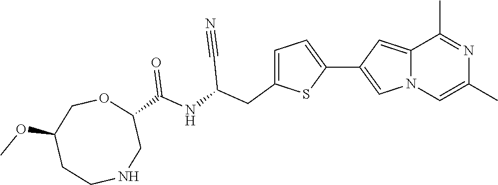 |
| A35 | 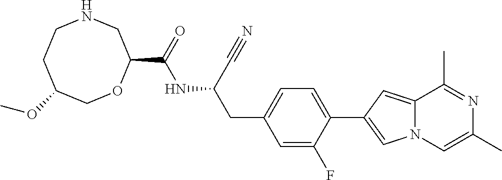 |
| A36 | 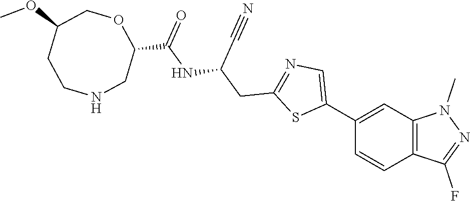 [SMILES: CO[C@@H]1CCNC[C@@H](C(=O)N[C@H](C#N)Cc2ncc(-c3ccc4c(F)nn(C)c4c3)s2)OC1] |
| A37 | 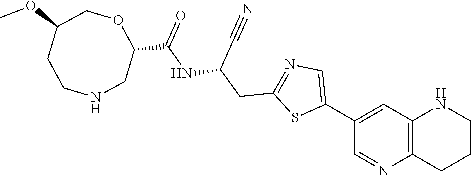 [SMILES: CO[C@@H]1CCNC[C@@H](C(=O)N[C@H](C#N)Cc2ncc(-c3cnc4c(c3)NCCC4)s2)OC1] |
| A38 | 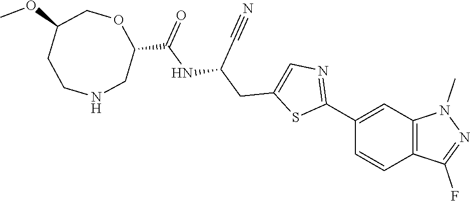 [SMILES: CO[C@@H]1CCNC[C@@H](C(=O)N[C@H](C#N)Cc2cnc(-c3ccc4c(F)nn(C)c4c3)s2)OC1] |
| A39 | 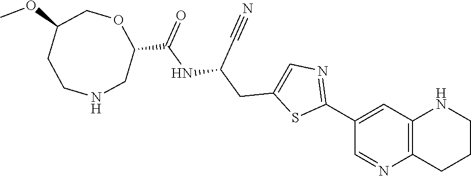 [SMILES: CO[C@@H]1CCNC[C@@H](C(=O)N[C@H](C#N)Cc2cnc(-c3cnc4c(c3)NCCC4)s2)OC1] |
| A40 | 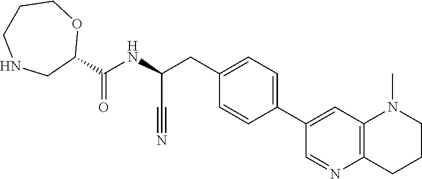 [SMILES: CN1CCCc2ncc(-c3ccc(C[C@@H](C#N)NC(=O)[C@@H]4CNCCCO4)cc3)cc21] |
| A41 | 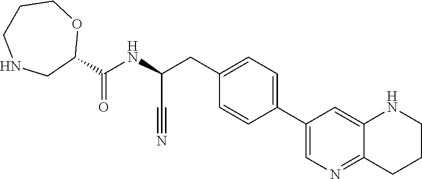 [SMILES: N#C[C@H](Cc1ccc(-c2cnc3c(c2)NCCC3)cc1)NC(=O)[C@@H]1CNCCCO1] |
| A42 | 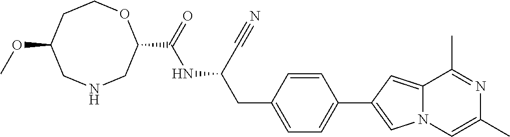 |
| A43 | 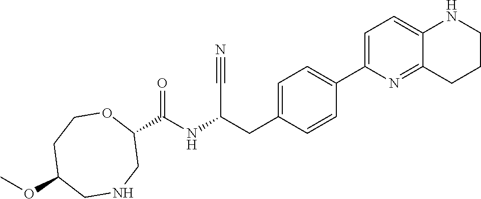 [SMILES: CO[C@H]1CCO[C@H](C(=O)N[C@H](C#N)Cc2ccc(-c3ccc4c(n3)CCCN4)cc2)CNC1] |
| A44 | 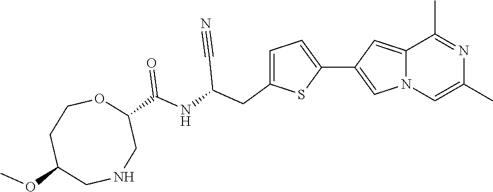 |
| A45* | 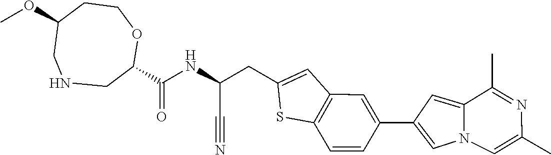 |
| A46* | 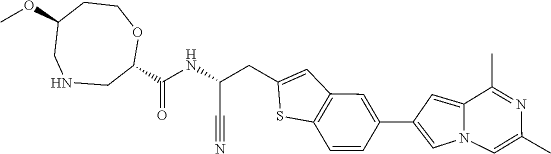 |
| A47 | 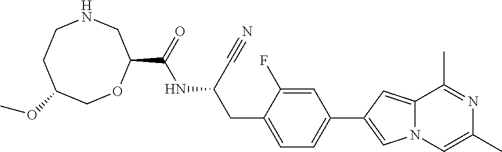 |
| A48 | 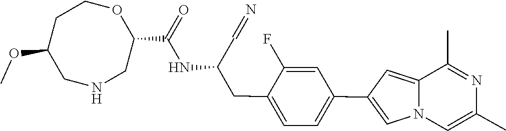 |
| A49 | 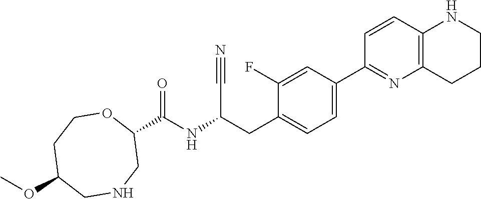 [SMILES: CO[C@H]1CCO[C@H](C(=O)N[C@H](C#N)Cc2ccc(-c3ccc4c(n3)CCCN4)cc2F)CNC1] |
| A50 | 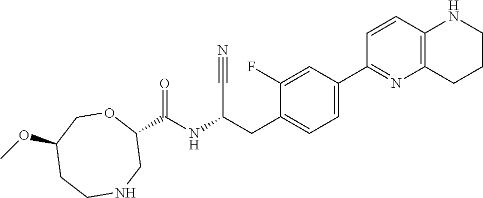 [SMILES: CO[C@@H]1CCNC[C@@H](C(=O)N[C@H](C#N)Cc2ccc(-c3ccc4c(n3)CCCN4)cc2F)OC1] |
| A51* | 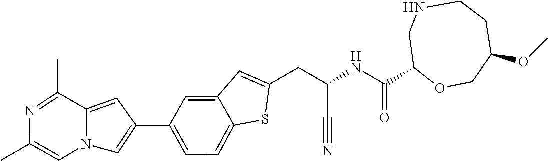 |
| A52* | 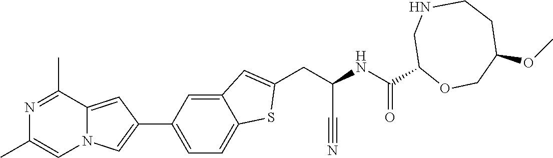 |
| A53 | 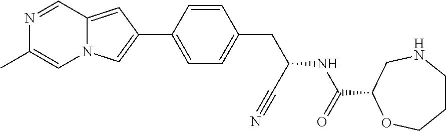 [SMILES: Cc1cn2cc(-c3ccc(C[C@@H](C#N)NC(=O)[C@@H]4CNCCCO4)cc3)cc2cn1] |
| A54 | 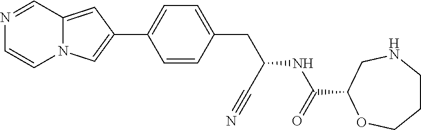 [SMILES: N#C[C@H](Cc1ccc(-c2cc3cnccn3c2)cc1)NC(=O)[C@@H]1CNCCCO1] |
| A55 | 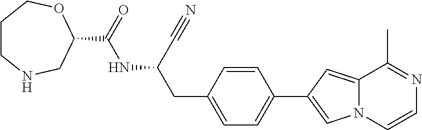 [SMILES: Cc1nccn2cc(-c3ccc(C[C@@H](C#N)NC(=O)[C@@H]4CNCCCO4)cc3)cc12] |
| A56 | 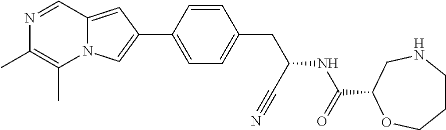 [SMILES: Cc1ncc2cc(-c3ccc(C[C@@H](C#N)NC(=O)[C@@H]4CNCCCO4)cc3)cn2c1C] |
| A57 | 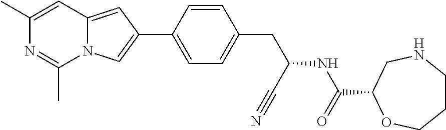 [SMILES: Cc1cc2cc(-c3ccc(C[C@@H](C#N)NC(=O)[C@@H]4CNCCCO4)cc3)cn2c(C)n1] |
| A58 | 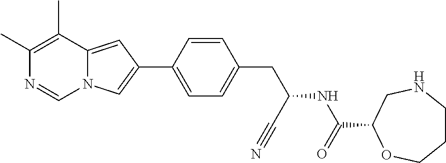 [SMILES: Cc1ncn2cc(-c3ccc(C[C@@H](C#N)NC(=O)[C@@H]4CNCCCO4)cc3)cc2c1C] |
| A59 | 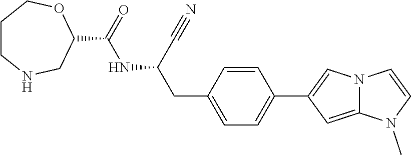 |
| A60 | 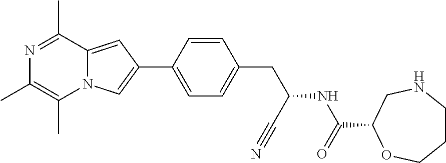 [SMILES: Cc1nc(C)c2cc(-c3ccc(C[C@@H](C#N)NC(=O)[C@@H]4CNCCCO4)cc3)cn2c1C] |
| A61 | 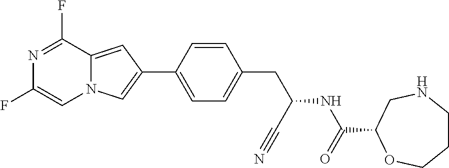 [SMILES: N#C[C@H](Cc1ccc(-c2cc3c(F)nc(F)cn3c2)cc1)NC(=O)[C@@H]1CNCCCO1] |
| A62 | 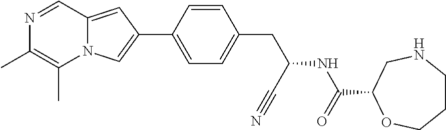 [SMILES: Cc1ncc2cc(-c3ccc(C[C@@H](C#N)NC(=O)[C@@H]4CNCCCO4)cc3)cn2c1C] |
| A63 | 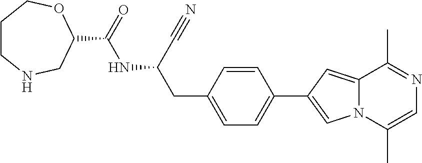 |
| A64 | 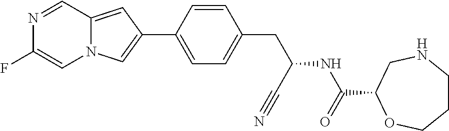 [SMILES: N#C[C@H](Cc1ccc(-c2cc3cnc(F)cn3c2)cc1)NC(=O)[C@@H]1CNCCCO1] |
| A65 | 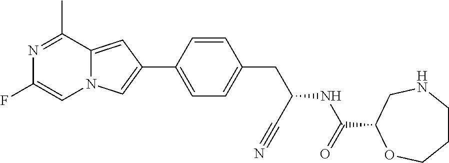 [SMILES: Cc1nc(F)cn2cc(-c3ccc(C[C@@H](C#N)NC(=O)[C@@H]4CNCCCO4)cc3)cc12] |
| A66 | 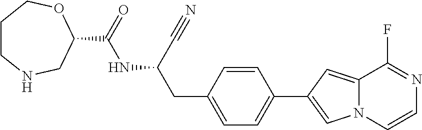 [SMILES: N#C[C@H](Cc1ccc(-c2cc3c(F)nccn3c2)cc1)NC(=O)[C@@H]1CNCCCO1] |
| A67 | 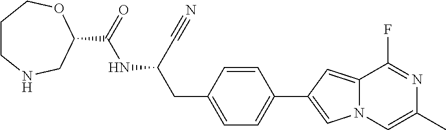 [SMILES: Cc1cn2cc(-c3ccc(C[C@@H](C#N)NC(=O)[C@@H]4CNCCCO4)cc3)cc2c(F)n1] |
| A68 | 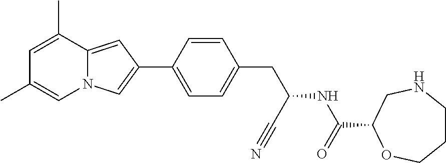 [SMILES: Cc1cc(C)c2cc(-c3ccc(C[C@@H](C#N)NC(=O)[C@@H]4CNCCCO4)cc3)cn2c1] |
| A69 | 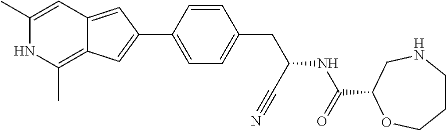 [SMILES: Cc1cc2cc(-c3ccc(C[C@@H](C#N)NC(=O)[C@@H]4CNCCCO4)cc3)cc-2c(C)[nH]1] |
| A70 | 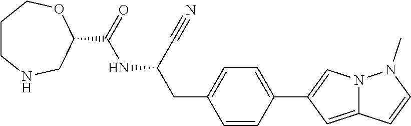 [SMILES: Cn1ccc2cc(-c3ccc(C[C@@H](C#N)NC(=O)[C@@H]4CNCCCO4)cc3)cn21] |
| A71 | 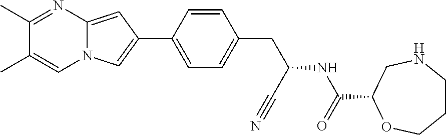 [SMILES: Cc1cn2cc(-c3ccc(C[C@@H](C#N)NC(=O)[C@@H]4CNCCCO4)cc3)cc2nc1C] |
| A72 | 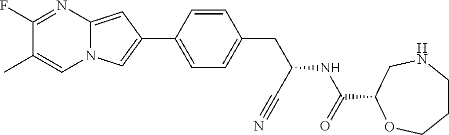 [SMILES: Cc1cn2cc(-c3ccc(C[C@@H](C#N)NC(=O)[C@@H]4CNCCCO4)cc3)cc2nc1F] |
| A73 | 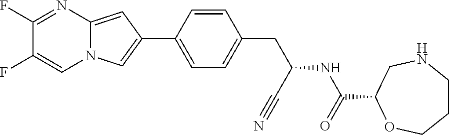 [SMILES: N#C[C@H](Cc1ccc(-c2cc3nc(F)c(F)cn3c2)cc1)NC(=O)[C@@H]1CNCCCO1] |
| A74 | 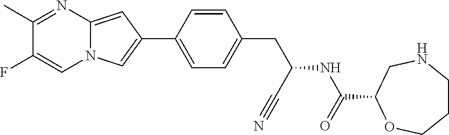 [SMILES: Cc1nc2cc(-c3ccc(C[C@@H](C#N)NC(=O)[C@@H]4CNCCCO4)cc3)cn2cc1F] |
| A75 | 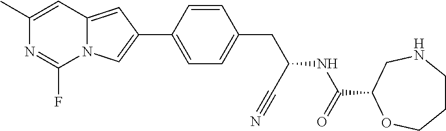 [SMILES: Cc1cc2cc(-c3ccc(C[C@@H](C#N)NC(=O)[C@@H]4CNCCCO4)cc3)cn2c(F)n1] |
| A76 | 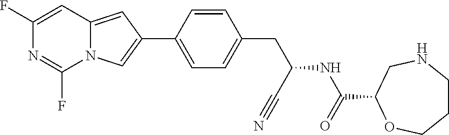 [SMILES: N#C[C@H](Cc1ccc(-c2cc3cc(F)nc(F)n3c2)cc1)NC(=O)[C@@H]1CNCCCO1] |
| A77 | 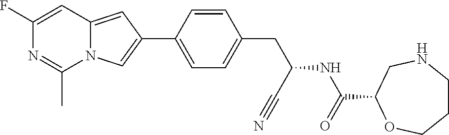 [SMILES: Cc1nc(F)cc2cc(-c3ccc(C[C@@H](C#N)NC(=O)[C@@H]4CNCCCO4)cc3)cn12] |
| A78 | 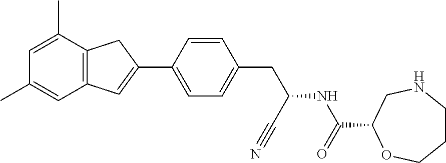 [SMILES: Cc1cc(C)c2c(c1)C=C(c1ccc(C[C@@H](C#N)NC(=O)[C@@H]3CNCCCO3)cc1)C2] |
| A79 | 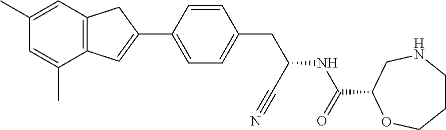 [SMILES: Cc1cc(C)c2c(c1)CC(c1ccc(C[C@@H](C#N)NC(=O)[C@@H]3CNCCCO3)cc1)=C2] |
| A80 | 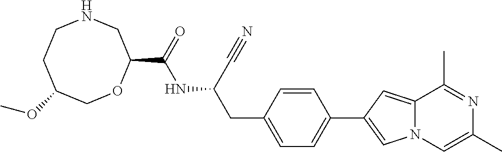 |
| A81 | 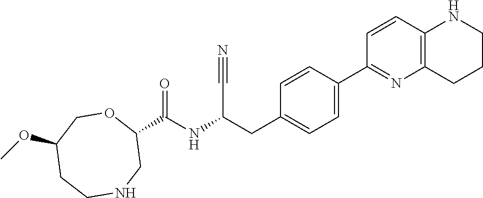 [SMILES: CO[C@@H]1CCNC[C@@H](C(=O)N[C@H](C#N)Cc2ccc(-c3ccc4c(n3)CCCN4)cc2)OC1] |
| A82 | 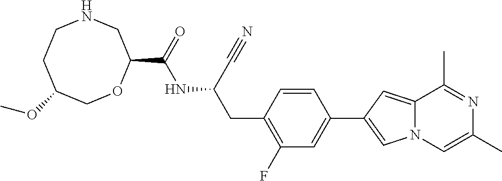 |
| A83 | 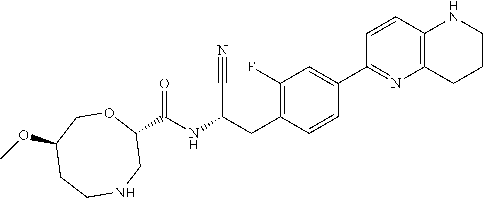 [SMILES: CO[C@@H]1CCNC[C@@H](C(=O)N[C@H](C#N)Cc2ccc(-c3ccc4c(n3)CCCN4)cc2F)OC1] |
| A84 | 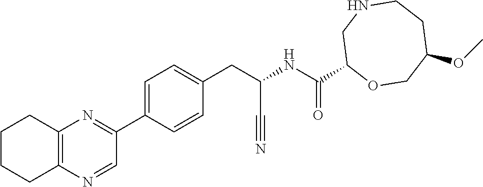 [SMILES: CO[C@@H]1CCNC[C@@H](C(=O)N[C@H](C#N)Cc2ccc(-c3cnc4c(n3)CCCC4)cc2)OC1] |
| A85 | 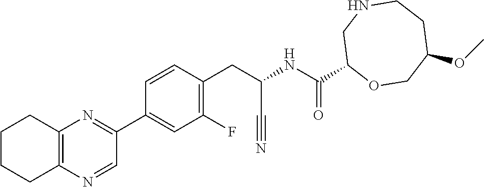 [SMILES: CO[C@@H]1CCNC[C@@H](C(=O)N[C@H](C#N)Cc2ccc(-c3cnc4c(n3)CCCC4)cc2F)OC1] |
| A86 | 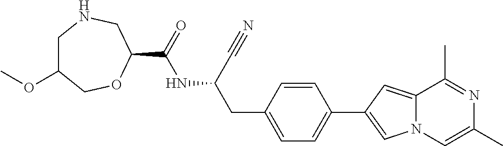 |
| A87 | 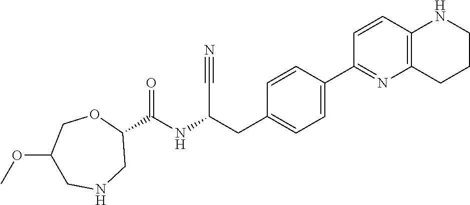 [SMILES: COC1CNC[C@@H](C(=O)N[C@H](C#N)Cc2ccc(-c3ccc4c(n3)CCCN4)cc2)OC1] |
| A88 | 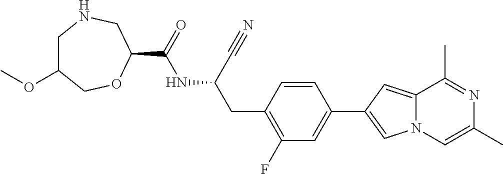 |
| A88-A | 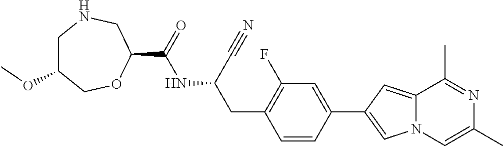 |
| A88-B | 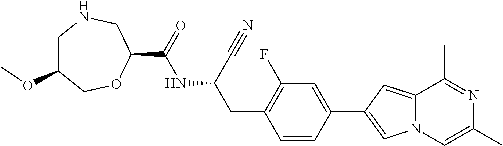 |
| A89 | 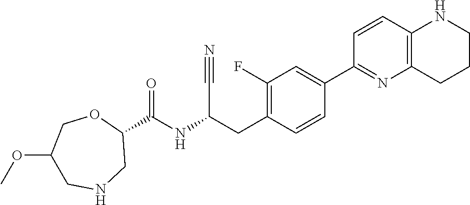 [SMILES: COC1CNC[C@@H](C(=O)N[C@H](C#N)Cc2ccc(-c3ccc4c(n3)CCCN4)cc2F)OC1] |
| A90 | 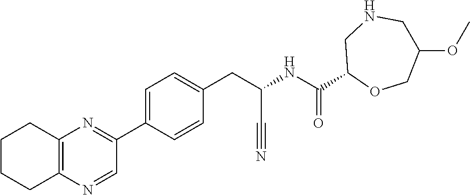 [SMILES: COC1CNC[C@@H](C(=O)N[C@H](C#N)Cc2ccc(-c3cnc4c(n3)CCCC4)cc2)OC1] |
| A91 | 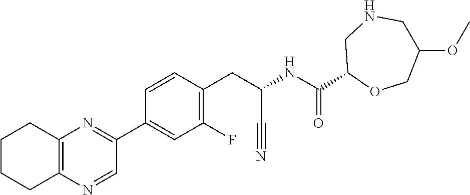 [SMILES: COC1CNC[C@@H](C(=O)N[C@H](C#N)Cc2ccc(-c3cnc4c(n3)CCCC4)cc2F)OC1] |
| A92 | 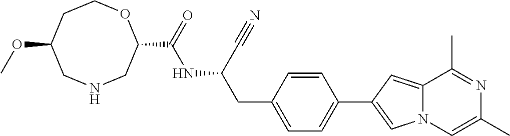 |
| A93 | 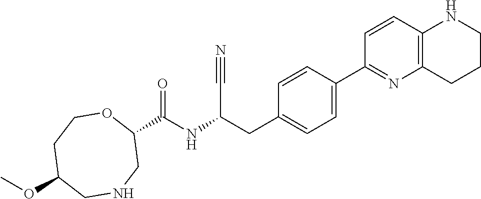 [SMILES: CO[C@H]1CCO[C@H](C(=O)N[C@H](C#N)Cc2ccc(-c3ccc4c(n3)CCCN4)cc2)CNC1] |
| A94 | 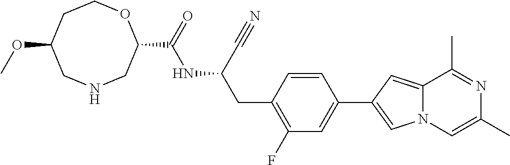 |
| A95 | 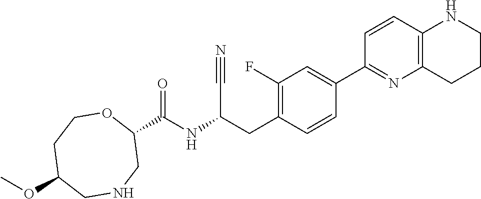 [SMILES: CO[C@H]1CCO[C@H](C(=O)N[C@H](C#N)Cc2ccc(-c3ccc4c(n3)CCCN4)cc2F)CNC1] |
| A96 | 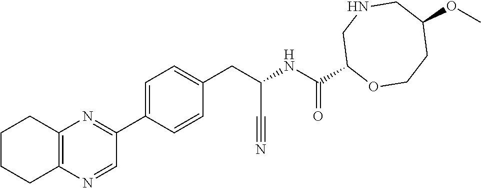 [SMILES: CO[C@H]1CCO[C@H](C(=O)N[C@H](C#N)Cc2ccc(-c3cnc4c(n3)CCCC4)cc2)CNC1] |
| A97 | 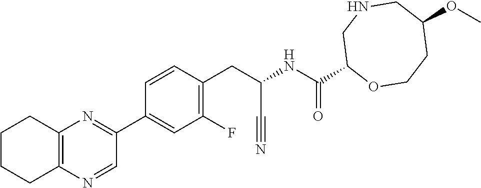 [SMILES: CO[C@H]1CCO[C@H](C(=O)N[C@H](C#N)Cc2ccc(-c3cnc4c(n3)CCCC4)cc2F)CNC1] |
| A98 | 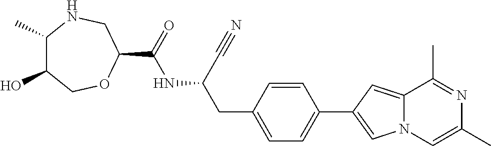 |
| A99 | 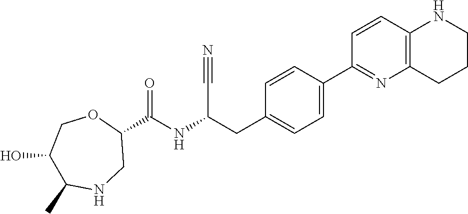 [SMILES: C[C@@H]1NC[C@@H](C(=O)N[C@H](C#N)Cc2ccc(-c3ccc4c(n3)CCCN4)cc2)OC[C@H]1O] |
| A100 | 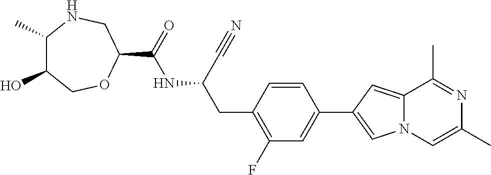 |
| A101 | 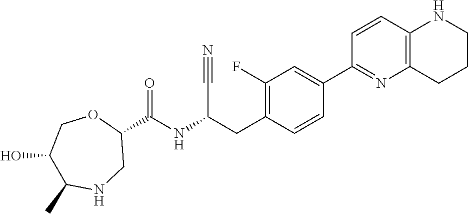 [SMILES: C[C@@H]1NC[C@@H](C(=O)N[C@H](C#N)Cc2ccc(-c3ccc4c(n3)CCCN4)cc2F)OC[C@H]1O] |
| A102 | 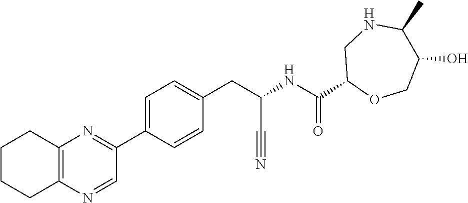 [SMILES: C[C@@H]1NC[C@@H](C(=O)N[C@H](C#N)Cc2ccc(-c3cnc4c(n3)CCCC4)cc2)OC[C@H]1O] |
| A103 | 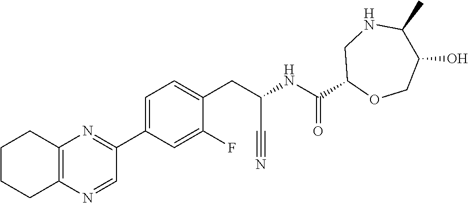 [SMILES: C[C@@H]1NC[C@@H](C(=O)N[C@H](C#N)Cc2ccc(-c3cnc4c(n3)CCCC4)cc2F)OC[C@H]1O] |
| A104 | 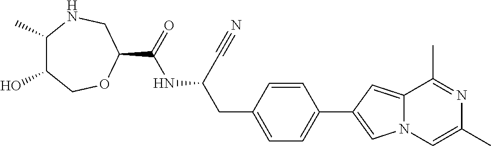 |
| A105 | 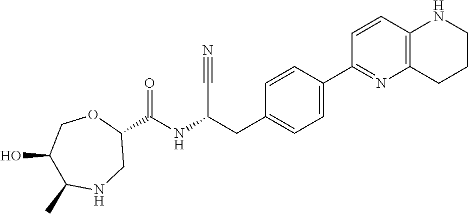 [SMILES: C[C@@H]1NC[C@@H](C(=O)N[C@H](C#N)Cc2ccc(-c3ccc4c(n3)CCCN4)cc2)OC[C@@H]1O] |
| A106 | 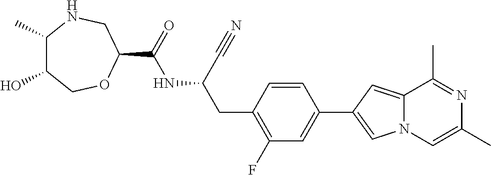 |
| A107 | 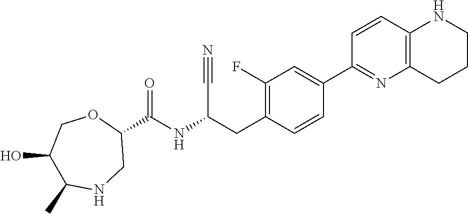 [SMILES: C[C@@H]1NC[C@@H](C(=O)N[C@H](C#N)Cc2ccc(-c3ccc4c(n3)CCCN4)cc2F)OC[C@@H]1O] |
| A108 | 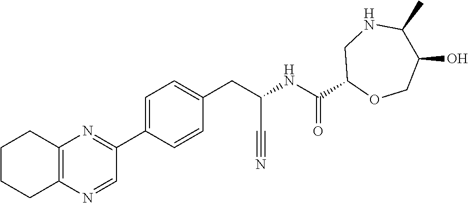 [SMILES: C[C@@H]1NC[C@@H](C(=O)N[C@H](C#N)Cc2ccc(-c3cnc4c(n3)CCCC4)cc2)OC[C@@H]1O] |
| A109 | 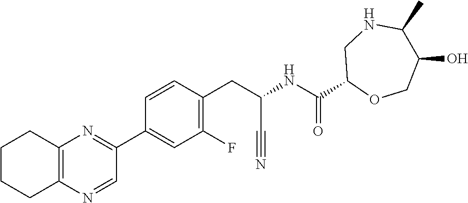 [SMILES: C[C@@H]1NC[C@@H](C(=O)N[C@H](C#N)Cc2ccc(-c3cnc4c(n3)CCCC4)cc2F)OC[C@@H]1O] |
| A110 | 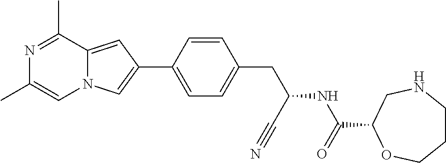 [SMILES: Cc1cn2cc(-c3ccc(C[C@@H](C#N)NC(=O)[C@@H]4CNCCCO4)cc3)cc2c(C)n1] |
| A111 | 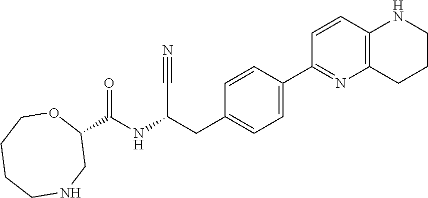 [SMILES: N#C[C@H](Cc1ccc(-c2ccc3c(n2)CCCN3)cc1)NC(=O)[C@@H]1CNCCCCO1] |
| A112 | 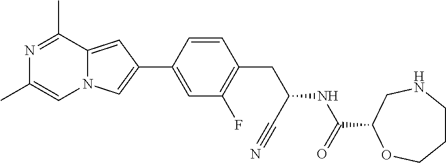 [SMILES: Cc1cn2cc(-c3ccc(C[C@@H](C#N)NC(=O)[C@@H]4CNCCCO4)c(F)c3)cc2c(C)n1] |
| A113 | 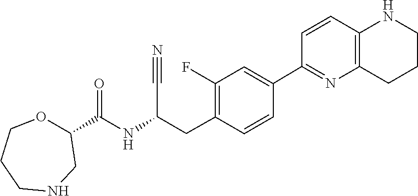 [SMILES: N#C[C@H](Cc1ccc(-c2ccc3c(n2)CCCN3)cc1F)NC(=O)[C@@H]1CNCCCO1] |
| A114 | 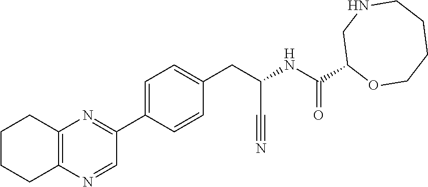 [SMILES: N#C[C@H](Cc1ccc(-c2cnc3c(n2)CCCC3)cc1)NC(=O)[C@@H]1CNCCCCO1] |
| A115 | 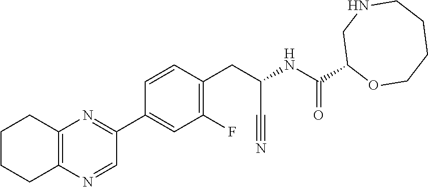 [SMILES: N#C[C@H](Cc1ccc(-c2cnc3c(n2)CCCC3)cc1F)NC(=O)[C@@H]1CNCCCCO1] |
| A116 | 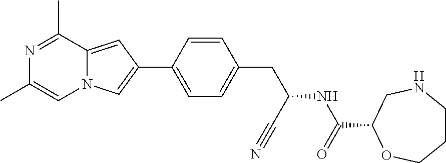 [SMILES: Cc1cn2cc(-c3ccc(C[C@@H](C#N)NC(=O)[C@@H]4CNCCCO4)cc3)cc2c(C)n1] |
| A117 | 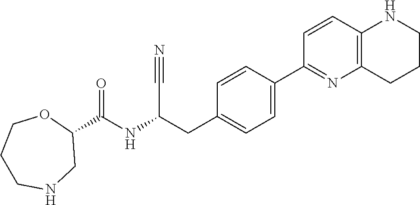 [SMILES: N#C[C@H](Cc1ccc(-c2ccc3c(n2)CCCN3)cc1)NC(=O)[C@@H]1CNCCCO1] |
| A118 | 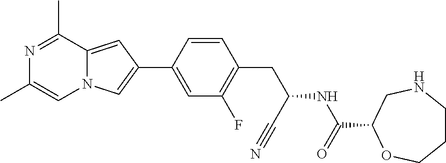 [SMILES: Cc1cn2cc(-c3ccc(C[C@@H](C#N)NC(=O)[C@@H]4CNCCCO4)c(F)c3)cc2c(C)n1] |
| A119 | 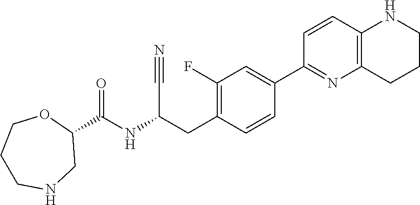 [SMILES: N#C[C@H](Cc1ccc(-c2ccc3c(n2)CCCN3)cc1F)NC(=O)[C@@H]1CNCCCO1] |
| A120 | 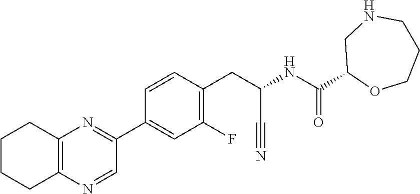 [SMILES: N#C[C@H](Cc1ccc(-c2cnc3c(n2)CCCC3)cc1F)NC(=O)[C@@H]1CNCCCO1] |
| A121 | 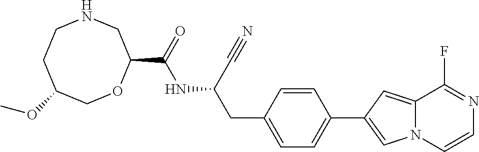 [SMILES: CO[C@@H]1CCNC[C@@H](C(=O)N[C@H](C#N)Cc2ccc(-c3cc4c(F)nccn4c3)cc2)OC1] |
| A122 | 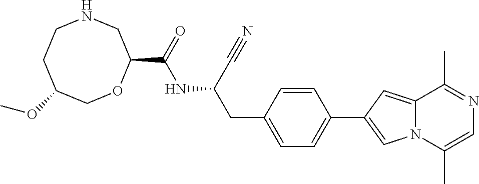 [SMILES: CO[C@@H]1CCNC[C@@H](C(=O)N[C@H](C#N)Cc2ccc(-c3cc4c(C)ncc(C)n4c3)cc2)OC1] |
| A123 | 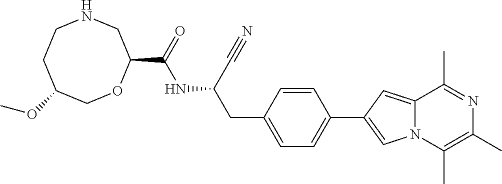 |
| A124 | 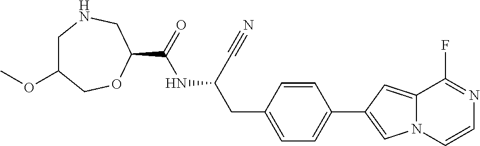 [SMILES: COC1CNC[C@@H](C(=O)N[C@H](C#N)Cc2ccc(-c3cc4c(F)nccn4c3)cc2)OC1] |
| A125 | 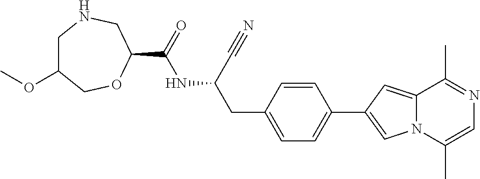 [SMILES: COC1CNC[C@@H](C(=O)N[C@H](C#N)Cc2ccc(-c3cc4c(C)ncc(C)n4c3)cc2)OC1] |
| A126 | 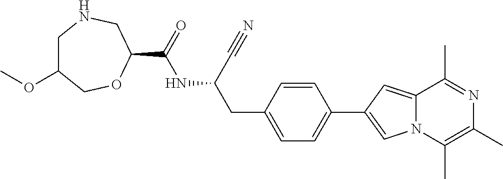 |
| A127 | 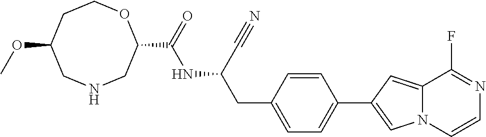 [SMILES: CO[C@H]1CCO[C@H](C(=O)N[C@H](C#N)Cc2ccc(-c3cc4c(F)nccn4c3)cc2)CNC1] |
| A128 | 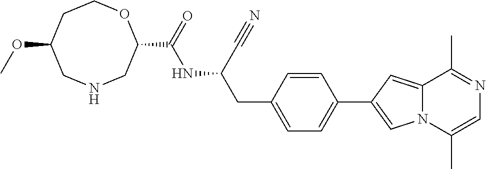 [SMILES: CO[C@H]1CCO[C@H](C(=O)N[C@H](C#N)Cc2ccc(-c3cc4c(C)ncc(C)n4c3)cc2)CNC1] |
| A129 | 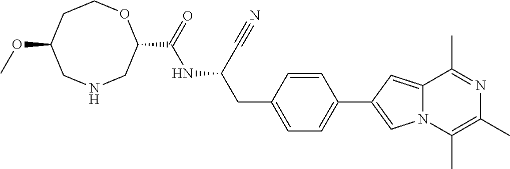 |
| A130 | 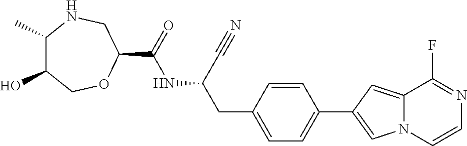 [SMILES: C[C@@H]1NC[C@@H](C(=O)N[C@H](C#N)Cc2ccc(-c3cc4c(F)nccn4c3)cc2)OC[C@H]1O] |
| A131 | 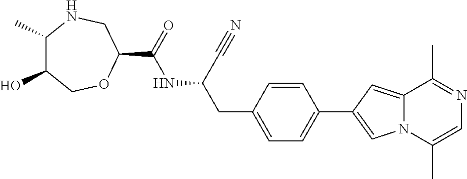 [SMILES: Cc1ncc(C)n2cc(-c3ccc(C[C@@H](C#N)NC(=O)[C@@H]4CN[C@@H](C)[C@H](O)CO4)cc3)cc12] |
| A132 | 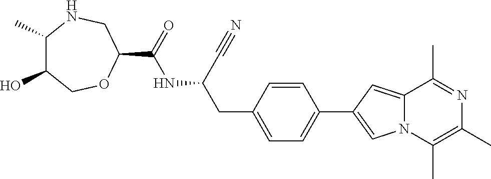 |
| A133 | 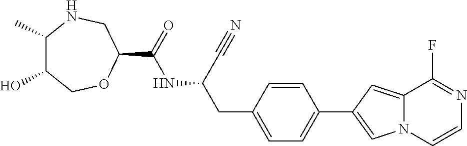 [SMILES: C[C@@H]1NC[C@@H](C(=O)N[C@H](C#N)Cc2ccc(-c3cc4c(F)nccn4c3)cc2)OC[C@@H]1O] |
| A134 | 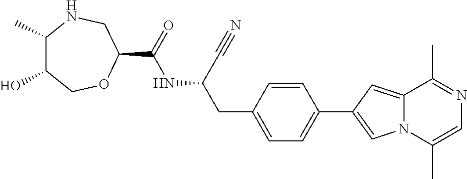 [SMILES: Cc1ncc(C)n2cc(-c3ccc(C[C@@H](C#N)NC(=O)[C@@H]4CN[C@@H](C)[C@@H](O)CO4)cc3)cc12] |
| A135 | 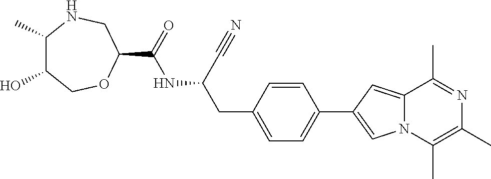 |
| A136 | 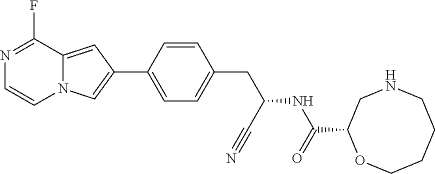 [SMILES: N#C[C@H](Cc1ccc(-c2cc3c(F)nccn3c2)cc1)NC(=O)[C@@H]1CNCCCCO1] |
| A137 | 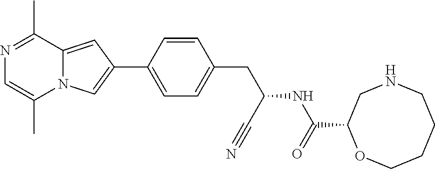 [SMILES: Cc1ncc(C)n2cc(-c3ccc(C[C@@H](C#N)NC(=O)[C@@H]4CNCCCCO4)cc3)cc12] |
| A138 | 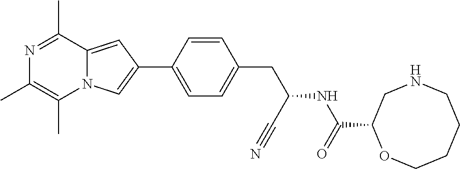 [SMILES: Cc1nc(C)c2cc(-c3ccc(C[C@@H](C#N)NC(=O)[C@@H]4CNCCCCO4)cc3)cn2c1C] |
| A139 | 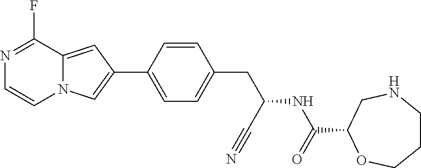 [SMILES: N#C[C@H](Cc1ccc(-c2cc3c(F)nccn3c2)cc1)NC(=O)[C@@H]1CNCCCO1] |
| A140 | 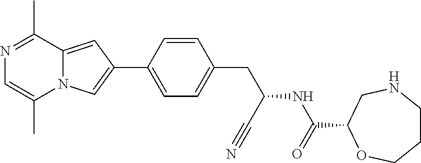 [SMILES: Cc1ncc(C)n2cc(-c3ccc(C[C@@H](C#N)NC(=O)[C@@H]4CNCCCO4)cc3)cc12] |
| A141 | 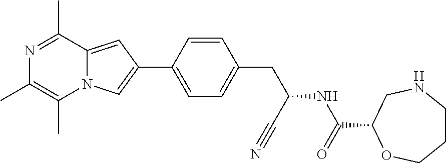 [SMILES: Cc1nc(C)c2cc(-c3ccc(C[C@@H](C#N)NC(=O)[C@@H]4CNCCCO4)cc3)cn2c1C] |
| A142 | 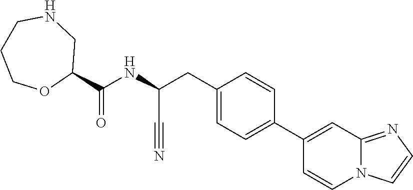 [SMILES: N#C[C@H](Cc1ccc(-c2ccn3ccnc3c2)cc1)NC(=O)[C@@H]1CNCCCO1] |
| A143 | 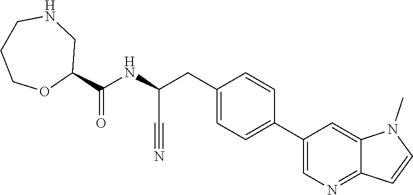 [SMILES: Cn1ccc2ncc(-c3ccc(C[C@@H](C#N)NC(=O)[C@@H]4CNCCCO4)cc3)cc21] |
| A144 | 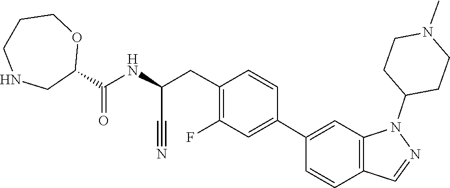 [SMILES: CN1CCC(n2ncc3ccc(-c4ccc(C[C@@H](C#N)NC(=O)[C@@H]5CNCCCO5)c(F)c4)cc32)CC1] |
| A145 | 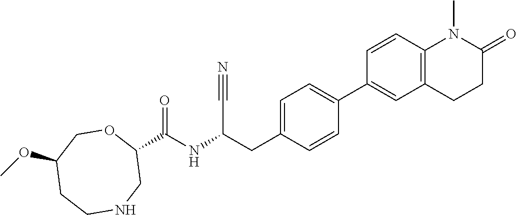 [SMILES: CO[C@@H]1CCNC[C@@H](C(=O)N[C@H](C#N)Cc2ccc(-c3ccc4c(c3)CCC(=O)N4C)cc2)OC1] |
| A146 | 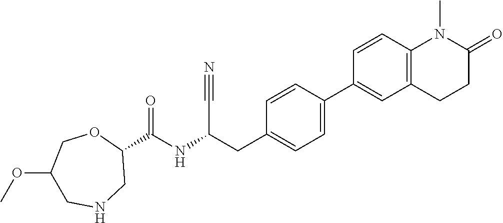 [SMILES: COC1CNC[C@@H](C(=O)N[C@H](C#N)Cc2ccc(-c3ccc4c(c3)CCC(=O)N4C)cc2)OC1] |
| A147 | 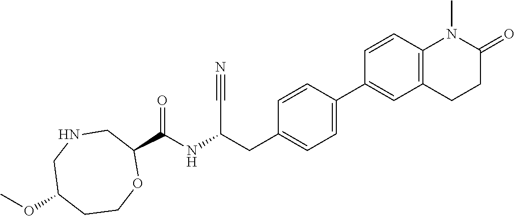 [SMILES: CO[C@H]1CCO[C@H](C(=O)N[C@H](C#N)Cc2ccc(-c3ccc4c(c3)CCC(=O)N4C)cc2)CNC1] |
| A148 | 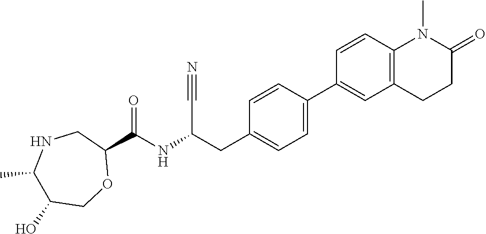 [SMILES: C[C@@H]1NC[C@@H](C(=O)N[C@H](C#N)Cc2ccc(-c3ccc4c(c3)CCC(=O)N4C)cc2)OC[C@@H]1O] |
| A149 | 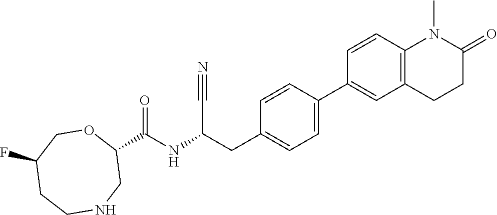 [SMILES: CN1C(=O)CCc2cc(-c3ccc(C[C@@H](C#N)NC(=O)[C@@H]4CNCC[C@@H](F)CO4)cc3)ccc21] |
| A150 | 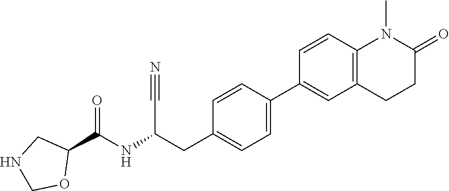 [SMILES: CN1C(=O)CCc2cc(-c3ccc(C[C@@H](C#N)NC(=O)[C@@H]4CNCO4)cc3)ccc21] |
| A151 | 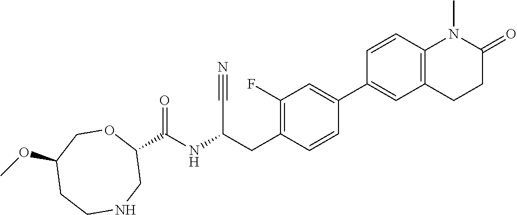 [SMILES: CO[C@@H]1CCNC[C@@H](C(=O)N[C@H](C#N)Cc2ccc(-c3ccc4c(c3)CCC(=O)N4C)cc2F)OC1] |
| A152 | 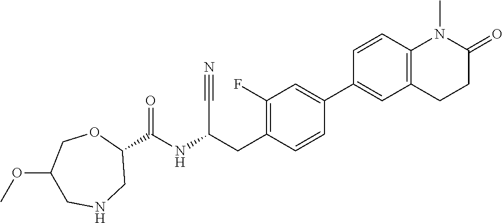 [SMILES: COC1CNC[C@@H](C(=O)N[C@H](C#N)Cc2ccc(-c3ccc4c(c3)CCC(=O)N4C)cc2F)OC1] |
| A153 | 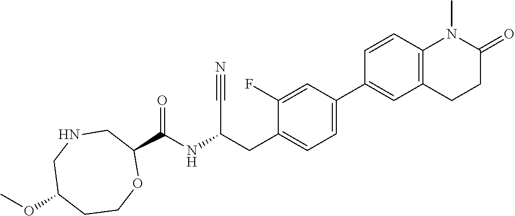 [SMILES: CO[C@H]1CCO[C@H](C(=O)N[C@H](C#N)Cc2ccc(-c3ccc4c(c3)CCC(=O)N4C)cc2F)CNC1] |
| A154 | 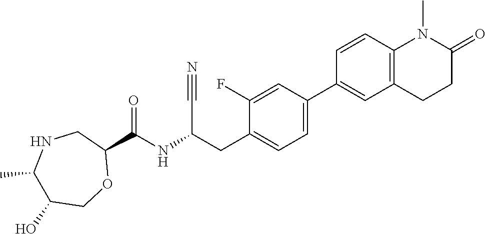 [SMILES: C[C@@H]1NC[C@@H](C(=O)N[C@H](C#N)Cc2ccc(-c3ccc4c(c3)CCC(=O)N4C)cc2F)OC[C@@H]1O] |
| A155 | 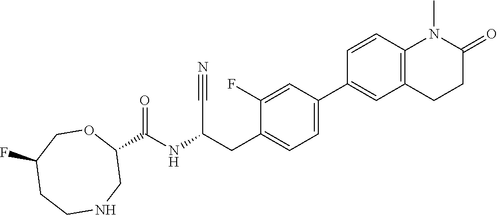 [SMILES: CN1C(=O)CCc2cc(-c3ccc(C[C@@H](C#N)NC(=O)[C@@H]4CNCC[C@@H](F)CO4)c(F)c3)ccc21] |
| A156 | 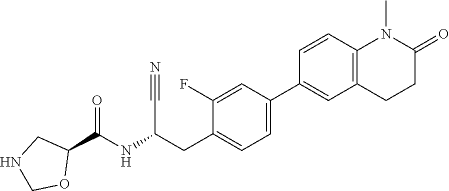 [SMILES: CN1C(=O)CCc2cc(-c3ccc(C[C@@H](C#N)NC(=O)[C@@H]4CNCO4)c(F)c3)ccc21] |
| A157 | 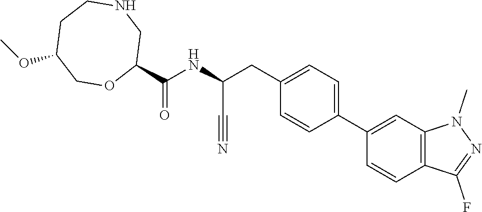 [SMILES: CO[C@@H]1CCNC[C@@H](C(=O)N[C@H](C#N)Cc2ccc(-c3ccc4c(F)nn(C)c4c3)cc2)OC1] |
| A158 | 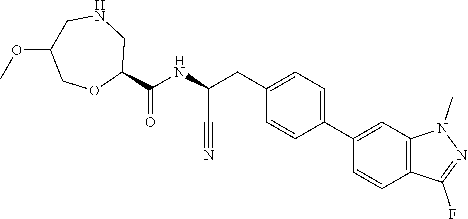 [SMILES: COC1CNC[C@@H](C(=O)N[C@H](C#N)Cc2ccc(-c3ccc4c(F)nn(C)c4c3)cc2)OC1] |
| A159 | 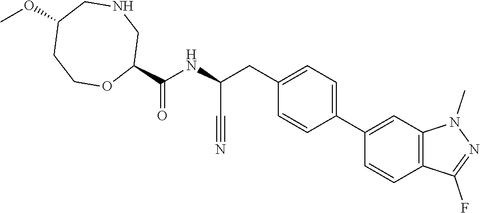 [SMILES: CO[C@H]1CCO[C@H](C(=O)N[C@H](C#N)Cc2ccc(-c3ccc4c(F)nn(C)c4c3)cc2)CNC1] |
| A160 | 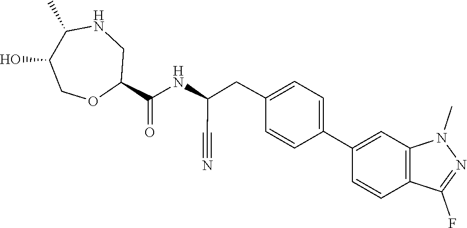 [SMILES: C[C@@H]1NC[C@@H](C(=O)N[C@H](C#N)Cc2ccc(-c3ccc4c(F)nn(C)c4c3)cc2)OC[C@@H]1O] |
| A161 | 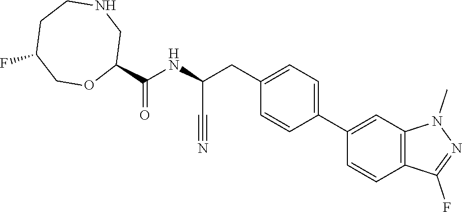 [SMILES: Cn1nc(F)c2ccc(-c3ccc(C[C@@H](C#N)NC(=O)[C@@H]4CNCC[C@@H](F)CO4)cc3)cc21] |
| A162 | 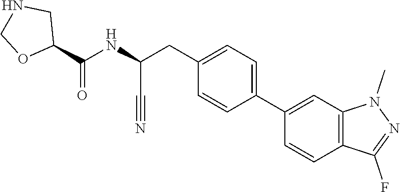 |
| A163 | 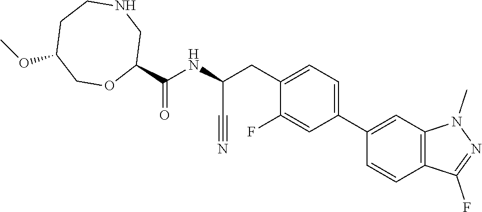 [SMILES: CO[C@@H]1CCNC[C@@H](C(=O)N[C@H](C#N)Cc2ccc(-c3ccc4c(F)nn(C)c4c3)cc2F)OC1] |
| A164 | 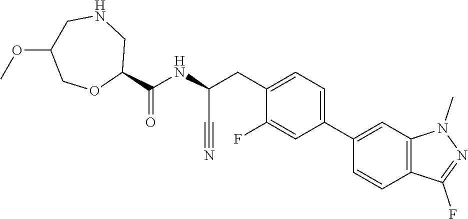 [SMILES: COC1CNC[C@@H](C(=O)N[C@H](C#N)Cc2ccc(-c3ccc4c(F)nn(C)c4c3)cc2F)OC1] |
| A165 | 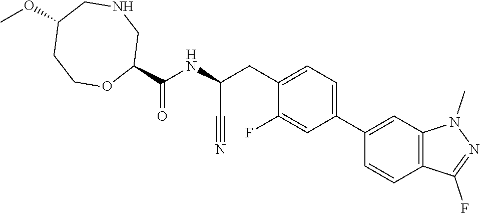 [SMILES: CO[C@H]1CCO[C@H](C(=O)N[C@H](C#N)Cc2ccc(-c3ccc4c(F)nn(C)c4c3)cc2F)CNC1] |
| A166 | 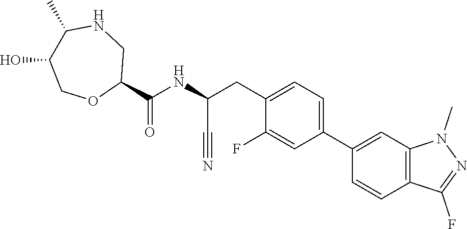 [SMILES: C[C@@H]1NC[C@@H](C(=O)N[C@H](C#N)Cc2ccc(-c3ccc4c(F)nn(C)c4c3)cc2F)OC[C@@H]1O] |
| A167 | 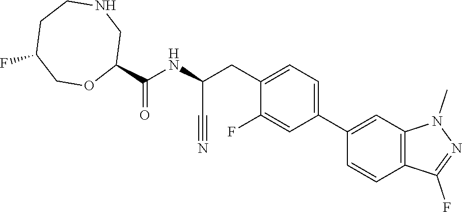 [SMILES: Cn1nc(F)c2ccc(-c3ccc(C[C@@H](C#N)NC(=O)[C@@H]4CNCC[C@@H](F)CO4)c(F)c3)cc21] |
| A168 | 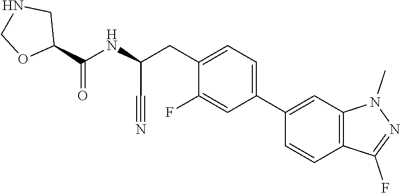 [SMILES: Cn1nc(F)c2ccc(-c3ccc(C[C@@H](C#N)NC(=O)[C@@H]4CNCO4)c(F)c3)cc21] |
| A169 | 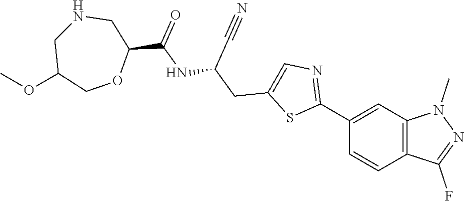 [SMILES: COC1CNC[C@@H](C(=O)N[C@H](C#N)Cc2cnc(-c3ccc4c(F)nn(C)c4c3)s2)OC1] |
| A170 | 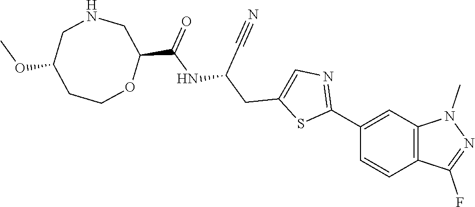 [SMILES: CO[C@H]1CCO[C@H](C(=O)N[C@H](C#N)Cc2cnc(-c3ccc4c(F)nn(C)c4c3)s2)CNC1] |
| A171 | 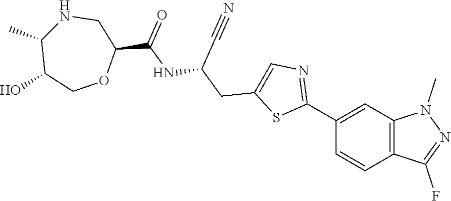 [SMILES: C[C@@H]1NC[C@@H](C(=O)N[C@H](C#N)Cc2cnc(-c3ccc4c(F)nn(C)c4c3)s2)OC[C@@H]1O] |
| A172 | 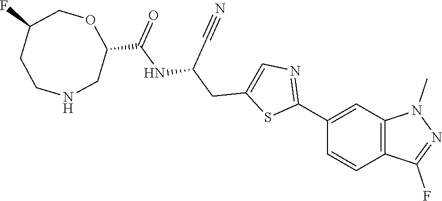 [SMILES: Cn1nc(F)c2ccc(-c3ncc(C[C@@H](C#N)NC(=O)[C@@H]4CNCC[C@@H](F)CO4)s3)cc21] |

*Represented stereochemistry is assumed.

In embodiments, provided herein is a compound of formula (II) selected from Compounds B7-B16 (includes B10-A and B10-B), or a pharmaceutically acceptable salt thereof, a deuterated form thereof, racemic form thereof, or stereoisomer thereof. In embodiments, a compound of formula (II) selected from Compounds B37-B330, or a pharmaceutically acceptable salt thereof, a deuterated form thereof, racemic form thereof, or stereoisomer thereof.

In embodiments, provided herein is a compound in Table B, or a pharmaceutically acceptable salt thereof, deuterated form thereof, racemic form thereof, or stereoisomer thereof.

In embodiments, provided herein is a compound in Table B, or a pharmaceutically acceptable salt thereof, or stereoisomer thereof.

In embodiments, provided herein is a compound in Table B, or a pharmaceutically acceptable salt thereof.

In one embodiment, provided herein is a compound set forth in Table B.

In some embodiments, provided herein is a pharmaceutically acceptable salt of a compound in Table B.

In embodiments, provided herein is a compound selected from Compounds B1, B3, or B4, or a pharmaceutically acceptable salt thereof, a deuterated form thereof, racemic form thereof, or stereoisomer thereof. In embodiments, provided herein is Compound B2, or a pharmaceutically acceptable salt thereof, or a deuterated form thereof. In embodiments, provided herein is a compound selected from Compound B31-B35, or a pharmaceutically acceptable salt thereof, a deuterated form thereof, racemic form thereof, or stereoisomer thereof.

In embodiments, the compound provided herein is Compound B9, or a pharmaceutically acceptable salt, a stereoisomer, or a deuterated form thereof.

In embodiments, the compound provided herein is Compound B10, or a pharmaceutically acceptable salt, a stereoisomer, or a deuterated form thereof.

In embodiments, the compound provided herein is Compound B11, or a pharmaceutically acceptable salt, a stereoisomer, or a deuterated form thereof

TABLE B
Various compounds of the invention

| Compound No. | Structure |
|---|---|
| B1 | 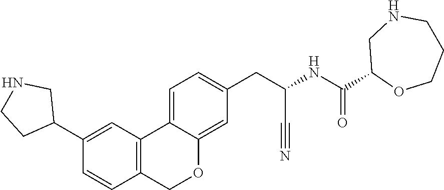 [SMILES: N#C[C@H](Cc1ccc2c(c1)OCc1ccc(C3CCNC3)cc1-2)NC(=O)[C@@H]1CNCCCO1] |
| B2 | 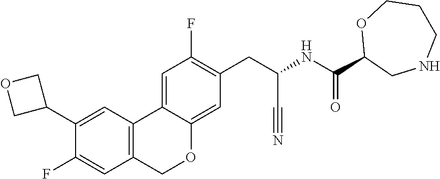 [SMILES: N#C[C@H](Cc1cc2c(cc1F)-c1cc(C3COC3)c(F)cc1CO2)NC(=O)[C@@H]1CNCCCO1] |
| B3 | 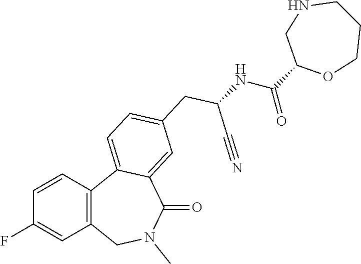 [SMILES: CN1Cc2cc(F)ccc2-c2ccc(C[C@@H](C#N)NC(=O)[C@@H]3CNCCCO3)cc2C1=O] |
| B4 | 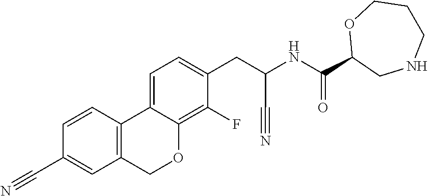 [SMILES: N#Cc1ccc2c(c1)COc1c-2ccc(CC(C#N)NC(=O)[C@@H]2CNCCCO2)c1F] |
| B7 | 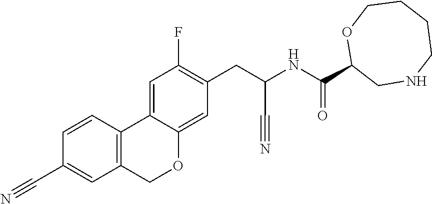 [SMILES: N#Cc1ccc2c(c1)COc1cc(CC(C#N)NC(=O)[C@@H]3CNCCCCO3)c(F)cc1-2] |
| B8* | 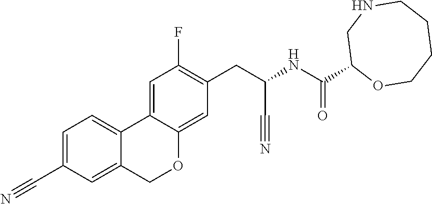 [SMILES: N#Cc1ccc2c(c1)COc1cc(C[C@@H](C#N)NC(=O)[C@@H]3CNCCCCO3)c(F)cc1-2] |
| B9* | 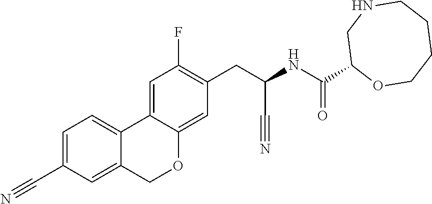 [SMILES: N#Cc1ccc2c(c1)COc1cc(C[C@H](C#N)NC(=O)[C@@H]3CNCCCCO3)c(F)cc1-2] |
| B10 | 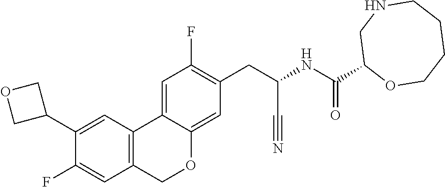 [SMILES: N#C[C@H](Cc1cc2c(cc1F)-c1cc(C3COC3)c(F)cc1CO2)NC(=O)[C@@H]1CNCCCCO1] |
| B10-A | 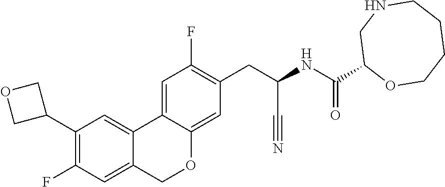 [SMILES: N#C[C@@H](Cc1cc2c(cc1F)-c1cc(C3COC3)c(F)cc1CO2)NC(=O)[C@@H]1CNCCCCO1] |
| B10-B | 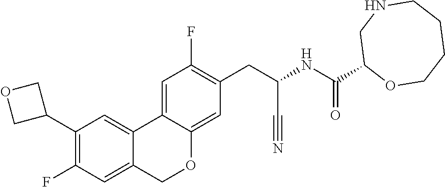 [SMILES: N#C[C@H](Cc1cc2c(cc1F)-c1cc(C3COC3)c(F)cc1CO2)NC(=O)[C@@H]1CNCCCCO1] |
| B11 | 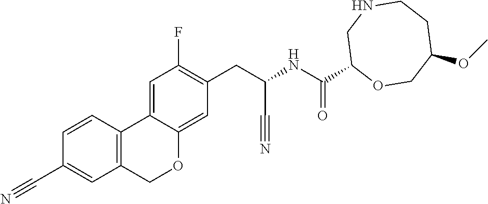 [SMILES: CO[C@@H]1CCNC[C@@H](C(=O)N[C@H](C#N)Cc2cc3c(cc2F)-c2ccc(C#N)cc2CO3)OC1] |
| B12 | 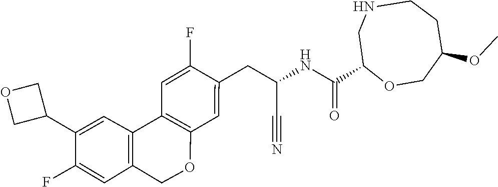 [SMILES: CO[C@@H]1CCNC[C@@H](C(=O)N[C@H](C#N)Cc2cc3c(cc2F)-c2cc(C4COC4)c(F)cc2CO3)OC1] |
| B13* | 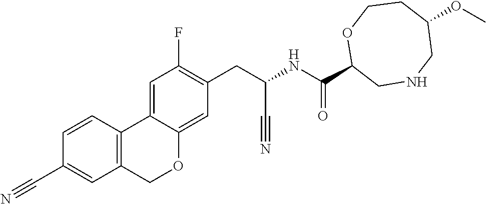 [SMILES: CO[C@H]1CCO[C@H](C(=O)N[C@H](C#N)Cc2cc3c(cc2F)-c2ccc(C#N)cc2CO3)CNC1] |
| B14* | 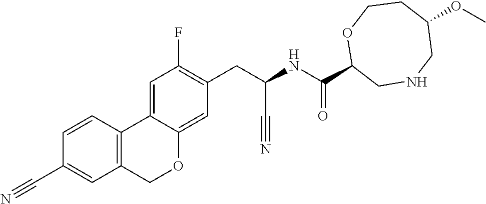 [SMILES: CO[C@H]1CCO[C@H](C(=O)N[C@@H](C#N)Cc2cc3c(cc2F)-c2ccc(C#N)cc2CO3)CNC1] |
| B15 | 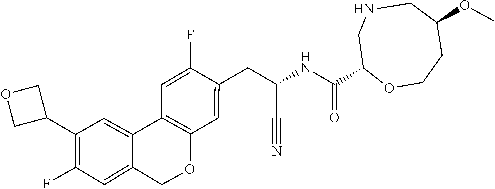 [SMILES: CO[C@H]1CCO[C@H](C(=O)N[C@H](C#N)Cc2cc3c(cc2F)-c2cc(C4COC4)c(F)cc2CO3)CNC1] |
| B16 | 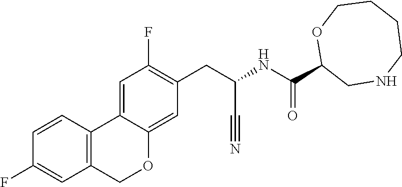 [SMILES: N#C[C@H](Cc1cc2c(cc1F)-c1ccc(F)cc1CO2)NC(=O)[C@@H]1CNCCCCO1] |
| B17 | 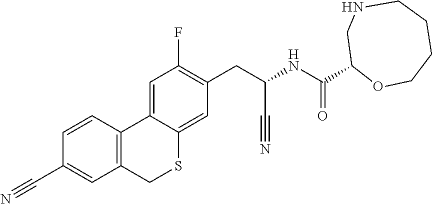 [SMILES: N#Cc1ccc2c(c1)CSc1cc(C[C@@H](C#N)NC(=O)[C@@H]3CNCCCCO3)c(F)cc1-2] |
| B18 | 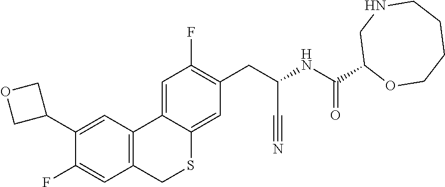 [SMILES: N#C[C@H](Cc1cc2c(cc1F)-c1cc(C3COC3)c(F)cc1CS2)NC(=O)[C@@H]1CNCCCCO1] |
| B19 | 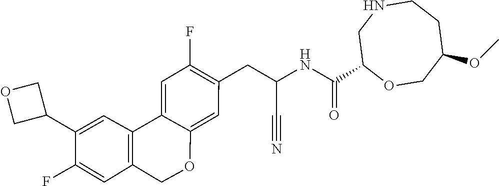 [SMILES: CO[C@@H]1CCNC[C@@H](C(=O)NC(C#N)Cc2cc3c(cc2F)-c2cc(C4COC4)c(F)cc2CO3)OC1] |
| B20 | 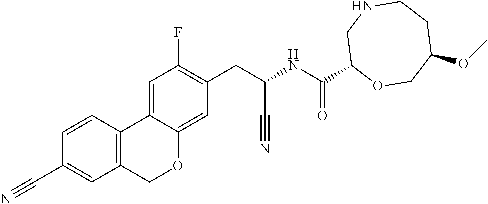 [SMILES: CO[C@@H]1CCNC[C@@H](C(=O)N[C@H](C#N)Cc2cc3c(cc2F)-c2ccc(C#N)cc2CO3)OC1] |
| B21 | 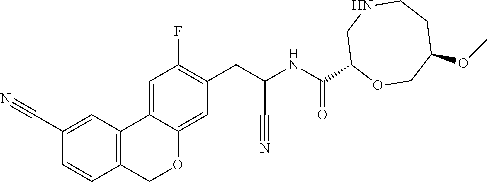 [SMILES: CO[C@@H]1CCNC[C@@H](C(=O)NC(C#N)Cc2cc3c(cc2F)-c2cc(C#N)ccc2CO3)OC1] |
| B22 | 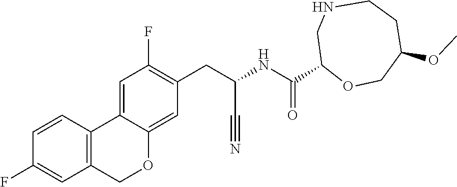 [SMILES: CO[C@@H]1CCNC[C@@H](C(=O)N[C@H](C#N)Cc2cc3c(cc2F)-c2ccc(F)cc2CO3)OC1] |
| B23 | 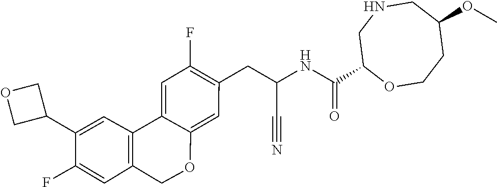 [SMILES: CO[C@H]1CCO[C@H](C(=O)NC(C#N)Cc2cc3c(cc2F)-c2cc(C4COC4)c(F)cc2CO3)CNC1] |
| B24 | 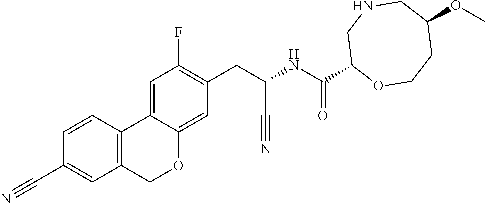 [SMILES: CO[C@H]1CCO[C@H](C(=O)N[C@H](C#N)Cc2cc3c(cc2F)-c2ccc(C#N)cc2CO3)CNC1] |
| B25 | 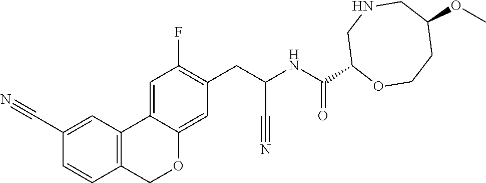 [SMILES: CO[C@H]1CCO[C@H](C(=O)NC(C#N)Cc2cc3c(cc2F)-c2cc(C#N)ccc2CO3)CNC1] |
| B26 | 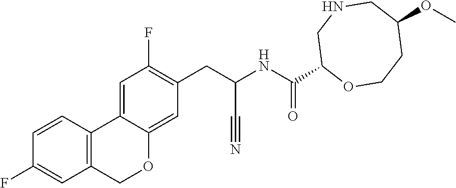 [SMILES: CO[C@H]1CCO[C@H](C(=O)NC(C#N)Cc2cc3c(cc2F)-c2ccc(F)cc2CO3)CNC1] |
| B27 | 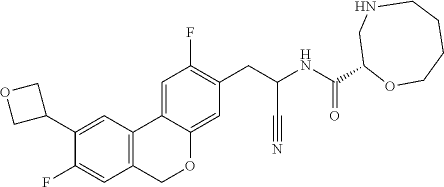 [SMILES: N#CC(Cc1cc2c(cc1F)-c1cc(C3COC3)c(F)cc1CO2)NC(=O)[C@@H]1CNCCCCO1] |
| B28 | 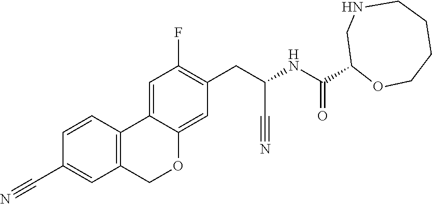 [SMILES: N#Cc1ccc2c(c1)COc1cc(C[C@@H](C#N)NC(=O)[C@@H]3CNCCCCO3)c(F)cc1-2] |
| B29 | 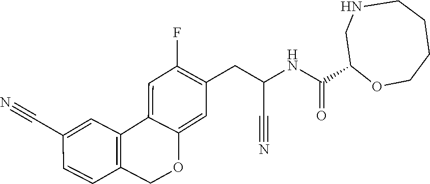 [SMILES: N#Cc1ccc2c(c1)-c1cc(F)c(CC(C#N)NC(=O)[C@@H]3CNCCCCO3)cc1OC2] |
| B30 | 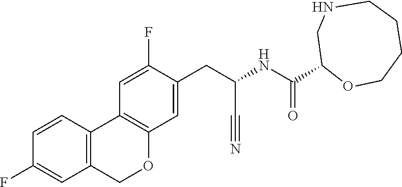 [SMILES: N#C[C@H](Cc1cc2c(cc1F)-c1ccc(F)cc1CO2)NC(=O)[C@@H]1CNCCCCO1] |
| B31 | 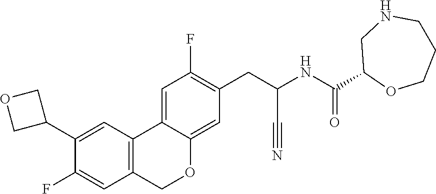 [SMILES: N#CC(Cc1cc2c(cc1F)-c1cc(C3COC3)c(F)cc1CO2)NC(=O)[C@@H]1CNCCCO1] |
| B32 | 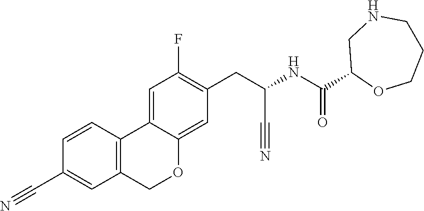 [SMILES: N#Cc1ccc2c(c1)COc1cc(C[C@@H](C#N)NC(=O)[C@@H]3CNCCCO3)c(F)cc1-2] |
| B33 | 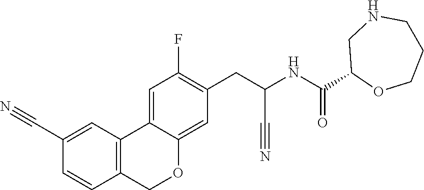 [SMILES: N#Cc1ccc2c(c1)-c1cc(F)c(CC(C#N)NC(=O)[C@@H]3CNCCCO3)cc1OC2] |
| B34 | 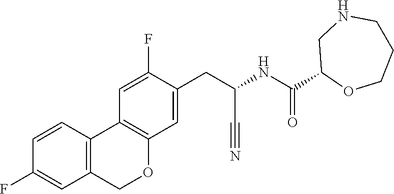 [SMILES: N#C[C@H](Cc1cc2c(cc1F)-c1ccc(F)cc1CO2)NC(=O)[C@@H]1CNCCCO1] |
| B35 | 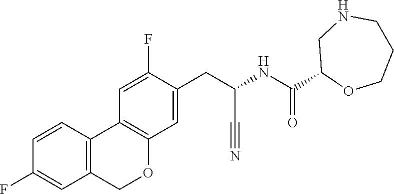 [SMILES: N#C[C@H](Cc1cc2c(cc1F)-c1ccc(F)cc1CO2)NC(=O)[C@@H]1CNCCCO1] |

*Represented stereochemistry is assumed.

In embodiments, provided herein is a compound of formula (III) selected from Compounds C₁-C₆, or a pharmaceutically acceptable salt thereof, a deuterated form thereof, racemic form thereof, or stereoisomer thereof. In embodiments, provided herein is a compound of formula (III) selected from Compounds C1-C34, or a pharmaceutically acceptable salt thereof, a deuterated form thereof, racemic form thereof, or stereoisomer thereof.

In embodiments, the compound provided herein is Compound C2, or a pharmaceutically acceptable salt, a stereoisomer, or a deuterated form thereof.

In embodiments, the compound provided herein is Compound C2-A, or a pharmaceutically acceptable salt, a stereoisomer, or a deuterated form thereof.

In embodiments, the compound provided herein is Compound C20, or a pharmaceutically acceptable salt, a stereoisomer, or a deuterated form thereof.

In embodiments, the compound provided herein is Compound C29, or a pharmaceutically acceptable salt, a stereoisomer, or a deuterated form thereof.

In embodiments, provided herein is a compound in Table C, or a pharmaceutically acceptable salt thereof, deuterated form thereof, racemic form thereof, or stereoisomer thereof.

In embodiments, provided herein is a compound in Table C, or a pharmaceutically acceptable salt thereof, or stereoisomer thereof.

In embodiments, provided herein is a compound in Table C, or a pharmaceutically acceptable salt thereof.

In one embodiment, provided herein is a compound set forth in Table C.

In some embodiments, provided herein is a pharmaceutically acceptable salt of a compound in Table C.

TABLE C
Various compounds of the invention

| Compound No. | Structure |
|---|---|
| C1 | 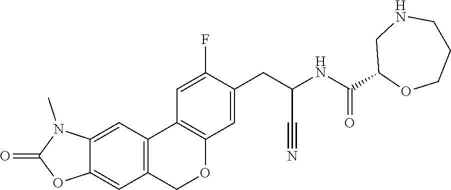 [SMILES: Cn1c(=O)oc2cc3c(cc21)-c1cc(F)c(CC(C#N)NC(=O)[C@@H]2CNCCCO2)cc1OC3] |
| C1-A | 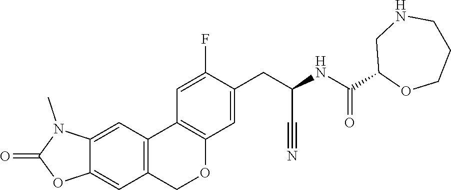 [SMILES: Cn1c(=O)oc2cc3c(cc21)-c1cc(F)c(C[C@H](C#N)NC(=O)[C@@H]2CNCCCO2)cc1OC3] |
| C1-B | 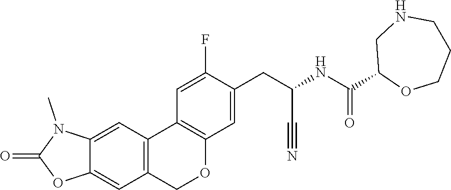 [SMILES: Cn1c(=O)oc2cc3c(cc21)-c1cc(F)c(C[C@@H](C#N)NC(=O)[C@@H]2CNCCCO2)cc1OC3] |
| C2 | 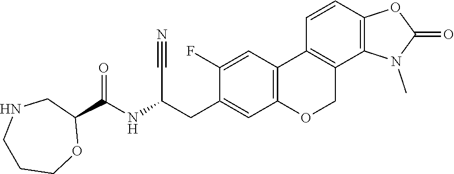 [SMILES: Cn1c(=O)oc2ccc3c(c21)COc1cc(C[C@@H](C#N)NC(=O)[C@@H]2CNCCCO2)c(F)cc1-3] |
| C2-A | 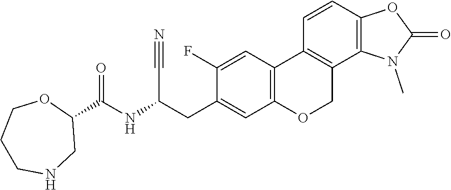 [SMILES: Cn1c(=O)oc2ccc3c(c21)COc1cc(C[C@@H](C#N)NC(=O)[C@@H]2CNCCCO2)c(F)cc1-3] |
| C2-B | 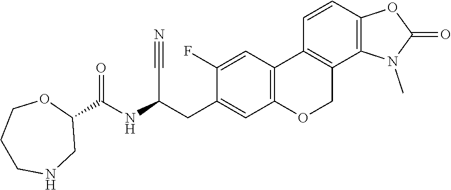 [SMILES: Cn1c(=O)oc2ccc3c(c21)COc1cc(C[C@H](C#N)NC(=O)[C@@H]2CNCCCO2)c(F)cc1-3] |
| C3 | 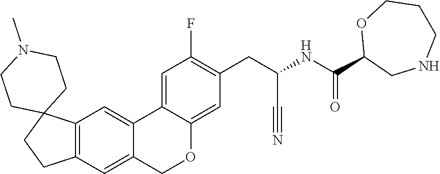 [SMILES: CN1CCC2(CCc3cc4c(cc32)-c2cc(F)c(C[C@@H](C#N)NC(=O)[C@@H]3CNCCCO3)cc2OC4)CC1] |
| C4 | 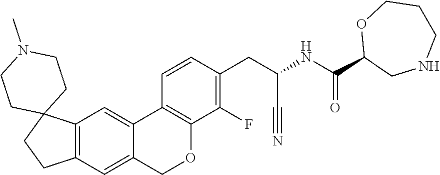 [SMILES: CN1CCC2(CCc3cc4c(cc32)-c2ccc(C[C@@H](C#N)NC(=O)[C@@H]3CNCCCO3)c(F)c2OC4)CC1] |
| C5* | 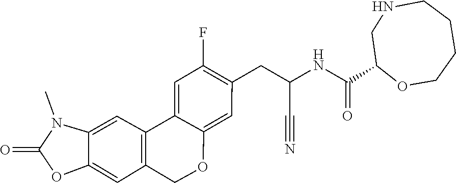 [SMILES: Cn1c(=O)oc2cc3c(cc21)-c1cc(F)c(CC(C#N)NC(=O)[C@@H]2CNCCCCO2)cc1OC3] |
| C6* | 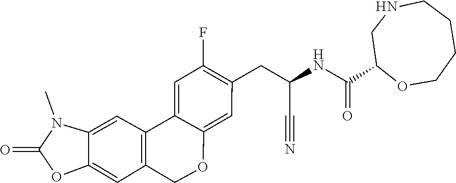 [SMILES: Cn1c(=O)oc2cc3c(cc21)-c1cc(F)c(C[C@H](C#N)NC(=O)[C@@H]2CNCCCCO2)cc1OC3] |
| C7 | 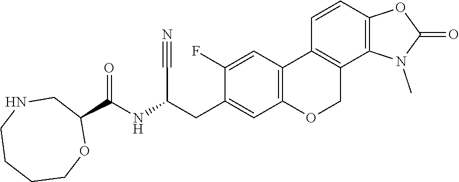 [SMILES: Cn1c(=O)oc2ccc3c(c21)COc1cc(C[C@@H](C#N)NC(=O)[C@@H]2CNCCCCO2)c(F)cc1-3] |
| C8 | 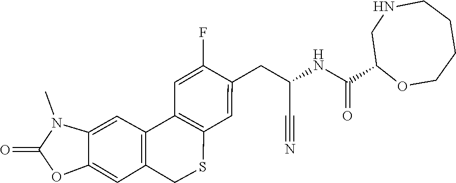 [SMILES: Cn1c(=O)oc2cc3c(cc21)-c1cc(F)c(C[C@@H](C#N)NC(=O)[C@@H]2CNCCCCO2)cc1SC3] |
| C9 | 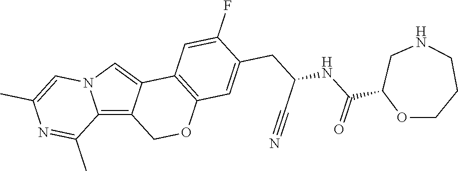 [SMILES: Cc1cn2cc3c(c2c(C)n1)COc1cc(C[C@@H](C#N)NC(=O)[C@@H]2CNCCCO2)c(F)cc1-3] |
| C10 | 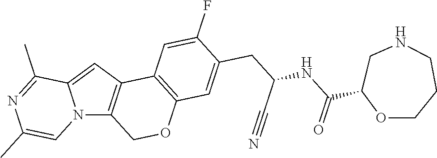 [SMILES: Cc1cn2c3c(cc2c(C)n1)-c1cc(F)c(C[C@@H](C#N)NC(=O)[C@@H]2CNCCCO2)cc1OC3] |
| C11 | 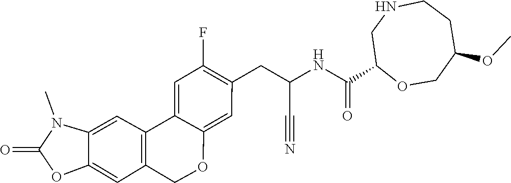 [SMILES: CO[C@@H]1CCNC[C@@H](C(=O)NC(C#N)Cc2cc3c(cc2F)-c2cc4c(cc2CO3)oc(=O)n4C)OC1] |
| C12 | 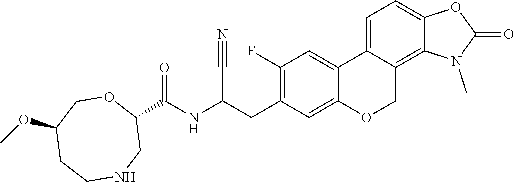 [SMILES: CO[C@@H]1CCNC[C@@H](C(=O)NC(C#N)Cc2cc3c(cc2F)-c2ccc4oc(=O)n(C)c4c2CO3)OC1] |
| C13 | 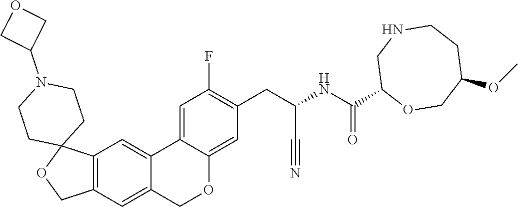 [SMILES: CO[C@@H]1CCNC[C@@H](C(=O)N[C@H](C#N)Cc2cc3c(cc2F)-c2cc4c(cc2CO3)COC42CCN(C3COC3)CC2)OC1] |
| C14 | 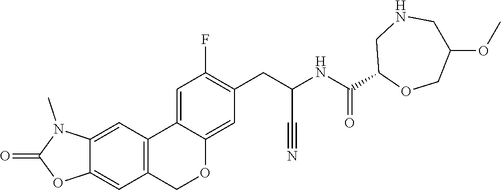 [SMILES: COC1CNC[C@@H](C(=O)NC(C#N)Cc2cc3c(cc2F)-c2cc4c(cc2CO3)oc(=O)n4C)OC1] |
| C15 | 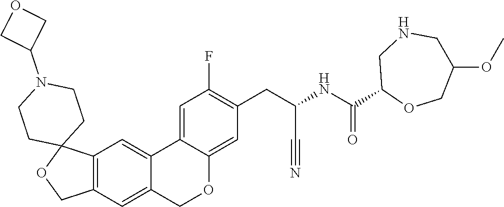 [SMILES: COC1CNC[C@@H](C(=O)N[C@H](C#N)Cc2cc3c(cc2F)-c2cc4c(cc2CO3)COC42CCN(C3COC3)CC2)OC1] |
| C16 | 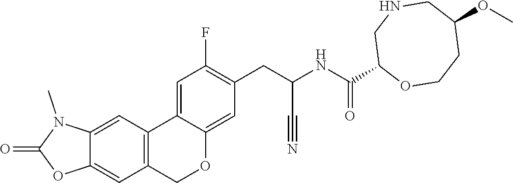 [SMILES: CO[C@H]1CCO[C@H](C(=O)NC(C#N)Cc2cc3c(cc2F)-c2cc4c(cc2CO3)oc(=O)n4C)CNC1] |
| C17 | 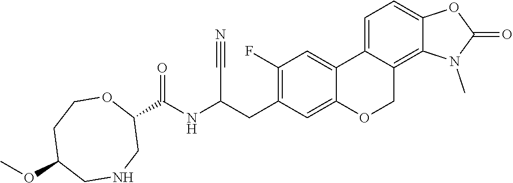 [SMILES: CO[C@H]1CCO[C@H](C(=O)NC(C#N)Cc2cc3c(cc2F)-c2ccc4oc(=O)n(C)c4c2CO3)CNC1] |
| C18 | 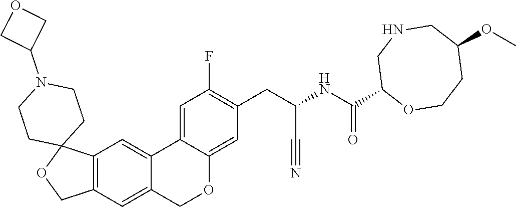 [SMILES: CO[C@H]1CCO[C@H](C(=O)N[C@H](C#N)Cc2cc3c(cc2F)-c2cc4c(cc2CO3)COC42CCN(C3COC3)CC2)CNC1] |
| C19 | 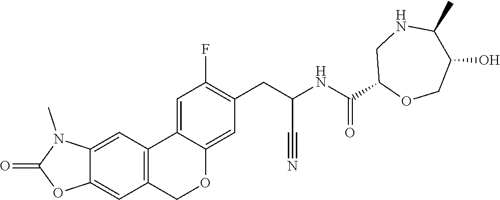 [SMILES: C[C@@H]1NC[C@@H](C(=O)NC(C#N)Cc2cc3c(cc2F)-c2cc4c(cc2CO3)oc(=O)n4C)OC[C@H]1O] |
| C20 | 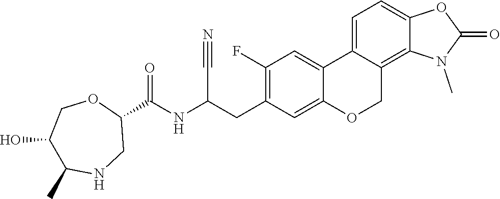 [SMILES: C[C@@H]1NC[C@@H](C(=O)NC(C#N)Cc2cc3c(cc2F)-c2ccc4oc(=O)n(C)c4c2CO3)OC[C@H]1O] |
| C21 | 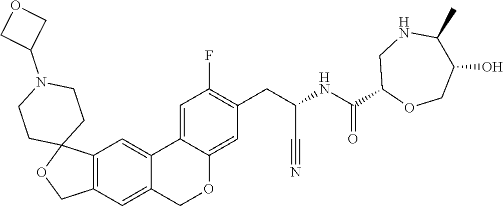 [SMILES: C[C@@H]1NC[C@@H](C(=O)N[C@H](C#N)Cc2cc3c(cc2F)-c2cc4c(cc2CO3)COC42CCN(C3COC3)CC2)OC[C@H]1O] |
| C22 | 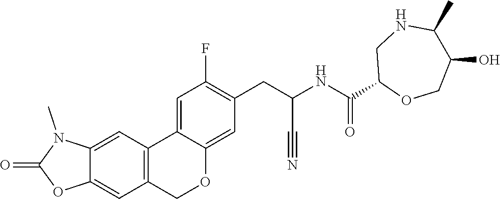 [SMILES: C[C@@H]1NC[C@@H](C(=O)NC(C#N)Cc2cc3c(cc2F)-c2cc4c(cc2CO3)oc(=O)n4C)OC[C@@H]1O] |
| C23 | 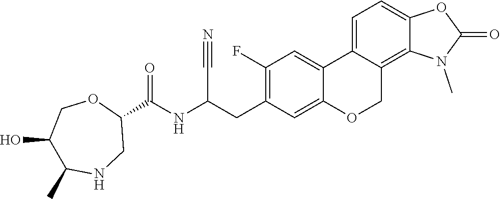 [SMILES: C[C@@H]1NC[C@@H](C(=O)NC(C#N)Cc2cc3c(cc2F)-c2ccc4oc(=O)n(C)c4c2CO3)OC[C@@H]1O] |
| C24 | 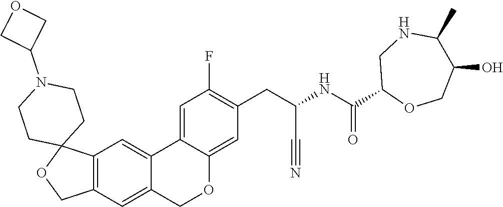 [SMILES: C[C@@H]1NC[C@@H](C(=O)N[C@H](C#N)Cc2cc3c(cc2F)-c2cc4c(cc2CO3)COC42CCN(C3COC3)CC2)OC[C@@H]1O] |
| C25 | 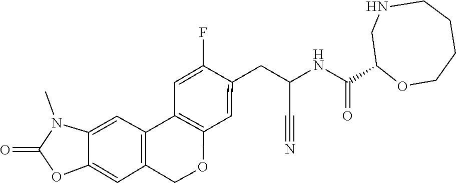 [SMILES: Cn1c(=O)oc2cc3c(cc21)-c1cc(F)c(CC(C#N)NC(=O)[C@@H]2CNCCCCO2)cc1OC3] |
| C26 | 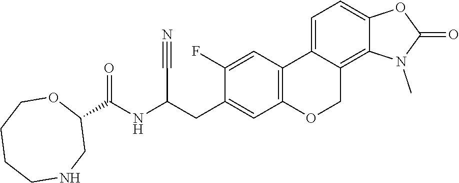 [SMILES: Cn1c(=O)oc2ccc3c(c21)COc1cc(CC(C#N)NC(=O)[C@@H]2CNCCCCO2)c(F)cc1-3] |
| C27 | 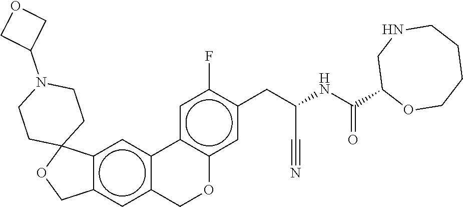 |
| C28 | 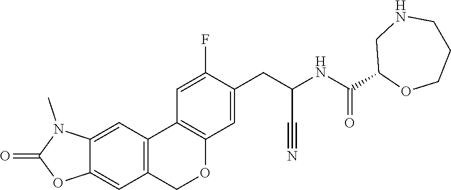 [SMILES: Cn1c(=O)oc2cc3c(cc21)-c1cc(F)c(CC(C#N)NC(=O)[C@@H]2CNCCCO2)cc1OC3] |
| C29 | 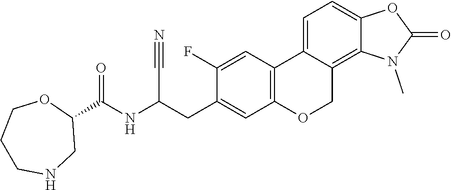 [SMILES: Cn1c(=O)oc2ccc3c(c21)COc1cc(CC(C#N)NC(=O)[C@@H]2CNCCCO2)c(F)cc1-3] |
| C30 | 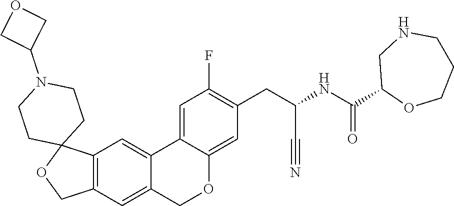 [SMILES: N#C[C@H](Cc1cc2c(cc1F)-c1cc3c(cc1CO2)COC31CCN(C2COC2)CC1)NC(=O)[C@@H]1CNCCCO1] |
| C31 | 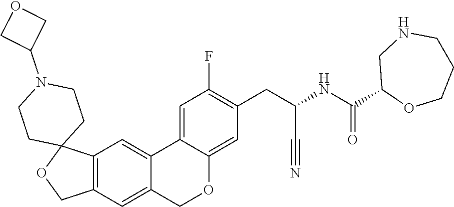 [SMILES: N#C[C@H](Cc1cc2c(cc1F)-c1cc3c(cc1CO2)COC31CCN(C2COC2)CC1)NC(=O)[C@@H]1CNCCCO1] |
| C32 | 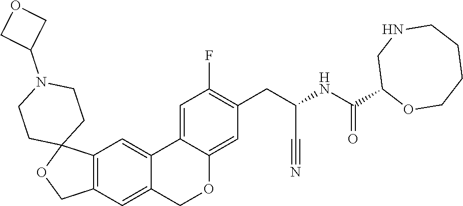 [SMILES: N#C[C@H](Cc1cc2c(cc1F)-c1cc3c(cc1CO2)COC31CCN(C2COC2)CC1)NC(=O)[C@@H]1CNCCCCO1] |
| C33 | 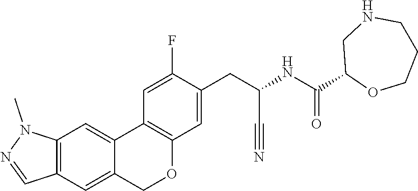 [SMILES: Cn1ncc2cc3c(cc21)-c1cc(F)c(C[C@@H](C#N)NC(=O)[C@@H]2CNCCCO2)cc1OC3] |
| C34 | 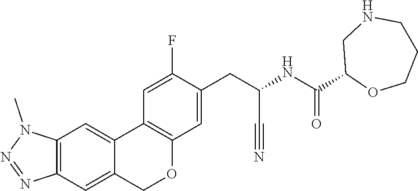 [SMILES: Cn1nnc2cc3c(cc21)-c1cc(F)c(C[C@@H](C#N)NC(=O)[C@@H]2CNCCCO2)cc1OC3] |

*Represented stereochemistry is assumed.

In embodiments, provided herein is a compound of formula (IV) selected from Compounds D1-D6, D8, or D9, or a pharmaceutically acceptable salt thereof, a deuterated form thereof, racemic form thereof, or stereoisomer thereof.

In embodiments, the compound provided herein is Compound D1, or a pharmaceutically acceptable salt, a stereoisomer, or a deuterated form thereof.

In embodiments, provided herein is a compound in Table D, or a pharmaceutically acceptable salt thereof, deuterated form thereof, racemic form thereof, or stereoisomer thereof.

In embodiments, provided herein is a compound in Table D, or a pharmaceutically acceptable salt thereof, or stereoisomer thereof.

In embodiments, provided herein is a compound in Table D, or a pharmaceutically acceptable salt thereof.

In one embodiment, provided herein is a compound set forth in Table D.

In some embodiments, provided herein is a pharmaceutically acceptable salt of a compound in Table D.

In embodiments, provided herein is Compound D7, or a pharmaceutically acceptable salt thereof, a deuterated form thereof, racemic form thereof, or stereoisomer thereof.

TABLE D
Various compounds of the invention

| Compound No. | Structure |
|---|---|
| D1 | 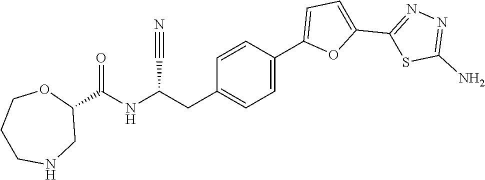 [SMILES: N#C[C@H](Cc1ccc(-c2ccc(-c3nnc(N)s3)o2)cc1)NC(=O)[C@@H]1CNCCCO1] |
| D2 | 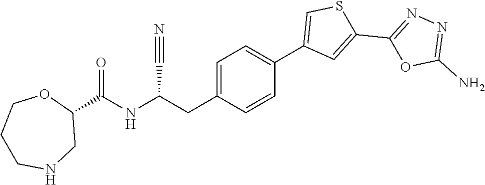 [SMILES: N#C[C@H](Cc1ccc(-c2csc(-c3nnc(N)o3)c2)cc1)NC(=O)[C@@H]1CNCCCO1] |
| D3 | 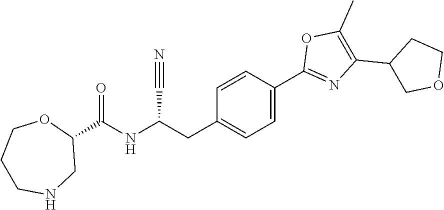 [SMILES: Cc1oc(-c2ccc(C[C@@H](C#N)NC(=O)[C@@H]3CNCCCO3)cc2)nc1C1CCOC1] |
| D4 | 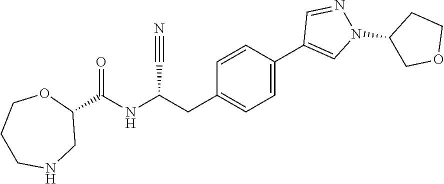 [SMILES: N#C[C@H](Cc1ccc(-c2cnn([C@@H]3CCOC3)c2)cc1)NC(=O)[C@@H]1CNCCCO1] |
| D5 | 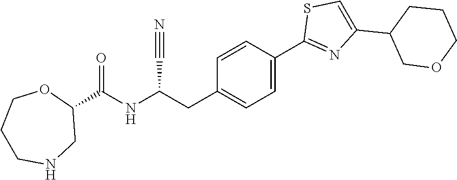 [SMILES: N#C[C@H](Cc1ccc(-c2nc(C3CCCOC3)cs2)cc1)NC(=O)[C@@H]1CNCCCO1] |
| D6 | 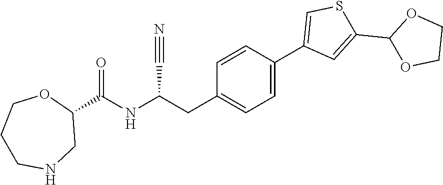 [SMILES: N#C[C@H](Cc1ccc(-c2csc(C3OCCO3)c2)cc1)NC(=O)[C@@H]1CNCCCO1] |
| D7 | 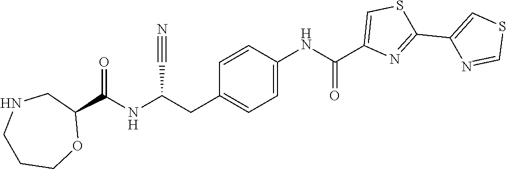 [SMILES: N#C[C@H](Cc1ccc(NC(=O)c2csc(-c3cscn3)n2)cc1)NC(=O)[C@@H]1CNCCCO1] |
| D8 | 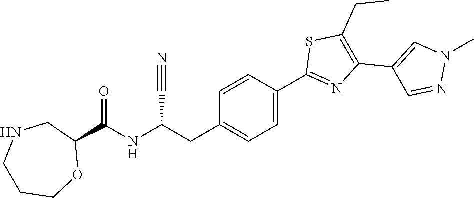 [SMILES: CCc1sc(-c2ccc(C[C@@H](C#N)NC(=O)[C@@H]3CNCCCO3)cc2)nc1-c1cnn(C)c1] |
| D9 | 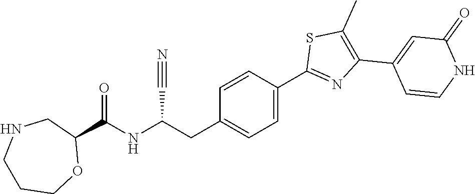 [SMILES: Cc1sc(-c2ccc(C[C@@H](C#N)NC(=O)[C@@H]3CNCCCO3)cc2)nc1-c1cc[nH]c(=O)c1] |

In embodiments, provided herein is a compound of formula (V) selected from Compounds E10, E12, E14, or E16, or a pharmaceutically acceptable salt thereof, a deuterated form thereof, racemic form thereof, or stereoisomer thereof.

In embodiments, the compound provided herein is Compound E12, or a pharmaceutically acceptable salt, a stereoisomer, or a deuterated form thereof.

In embodiments, provided herein is a compound in Table E, or a pharmaceutically acceptable salt thereof, deuterated form thereof, racemic form thereof, or stereoisomer thereof.

In embodiments, provided herein is a compound in Table E, or a pharmaceutically acceptable salt thereof, or stereoisomer thereof.

In embodiments, provided herein is a compound in Table E, or a pharmaceutically acceptable salt thereof.

In one embodiment, provided herein is a compound set forth in Table E.

In some embodiments, provided herein is a pharmaceutically acceptable salt of a compound in Table E.

TABLE E
Various compounds of the invention

| Compound No. | Structure |
|---|---|
| E10 | 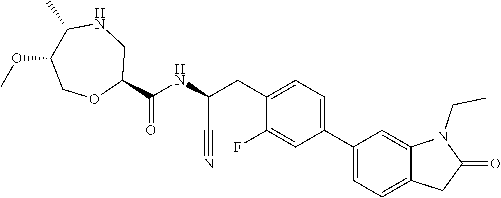 [SMILES: CCN1C(=O)Cc2ccc(-c3ccc(C[C@@H](C#N)NC(=O)[C@@H]4CN[C@@H](C)[C@@H](OC)CO4)c(F)c3)cc21] |
| E12 | 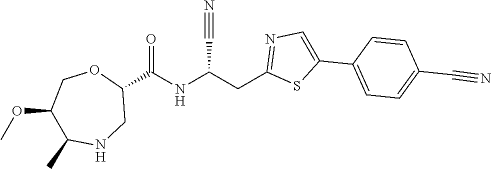 [SMILES: CO[C@H]1CO[C@H](C(=O)N[C@H](C#N)Cc2ncc(-c3ccc(C#N)cc3)s2)CN[C@H]1C] |
| E14 | 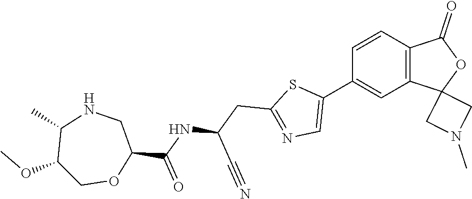 [SMILES: CO[C@H]1CO[C@H](C(=O)N[C@H](C#N)Cc2ncc(-c3ccc4c(c3)C3(CN(C)C3)OC4=O)s2)CN[C@H]1C] |
| E16 | 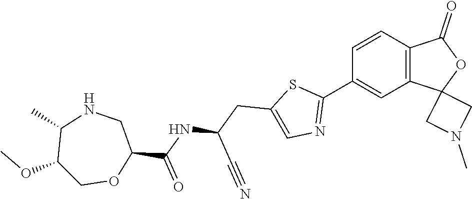 [SMILES: CO[C@H]1CO[C@H](C(=O)N[C@H](C#N)Cc2cnc(-c3ccc4c(c3)C3(CN(C)C3)OC4=O)s2)CN[C@H]1C] |

*Represented stereochemistry is assumed.

In embodiments, provided herein is a compound of formula (VI) selected from Compounds F1-F6, or a pharmaceutically acceptable salt thereof, a deuterated form thereof, racemic form thereof, or stereoisomer thereof. In embodiments, the compound is selected from Compounds F1-F30, or a pharmaceutically acceptable salt thereof, a deuterated form thereof, racemic form thereof, or stereoisomer thereof.

In embodiments, the compound provided herein is Compound F4, or a pharmaceutically acceptable salt, a stereoisomer, or a deuterated form thereof.

In embodiments, the compound provided herein is Compound F10, or a pharmaceutically acceptable salt, a stereoisomer, or a deuterated form thereof

In embodiments, provided herein is a compound in Table F, or a pharmaceutically acceptable salt thereof, deuterated form thereof, racemic form thereof, or stereoisomer thereof.

In embodiments, provided herein is a compound in Table F, or a pharmaceutically acceptable salt thereof, or stereoisomer thereof.

In embodiments, provided herein is a compound in Table F, or a pharmaceutically acceptable salt thereof.

In one embodiment, provided herein is a compound set forth in Table F.

In some embodiments, provided herein is a pharmaceutically acceptable salt of a compound in Table F.

Table F. Various compounds of the invention

TABLE F
Various compounds of the invention

| Compound No. | Structure |
|---|---|
| F1 | 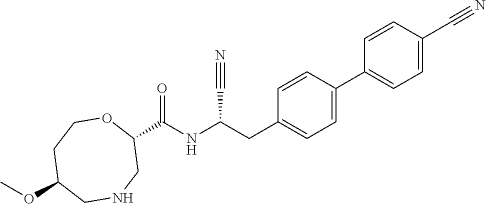 [SMILES: CO[C@H]1CCO[C@H](C(=O)N[C@H](C#N)Cc2ccc(-c3ccc(C#N)cc3)cc2)CNC1] |
| F2 | 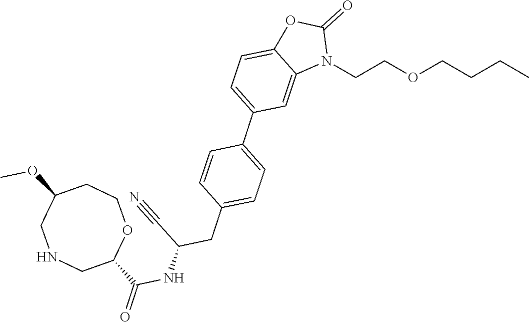 [SMILES: CCCCOCCn1c(=O)oc2ccc(-c3ccc(C[C@@H](C#N)NC(=O)[C@@H]4CNC[C@@H](OC)CCO4)cc3)cc21] |
| F3 | 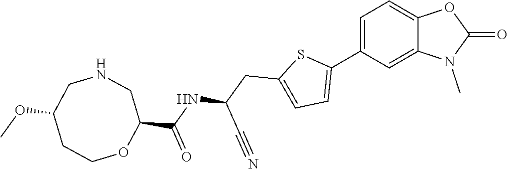 [SMILES: CO[C@H]1CCO[C@H](C(=O)N[C@H](C#N)Cc2ccc(-c3ccc4oc(=O)n(C)c4c3)s2)CNC1] |
| F4 | 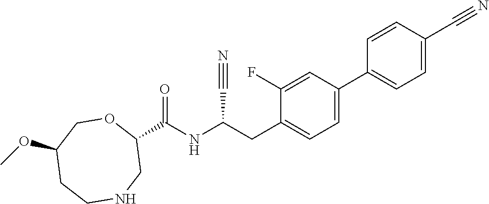 [SMILES: CO[C@@H]1CCNC[C@@H](C(=O)N[C@H](C#N)Cc2ccc(-c3ccc(C#N)cc3)cc2F)OC1] |
| F5 | 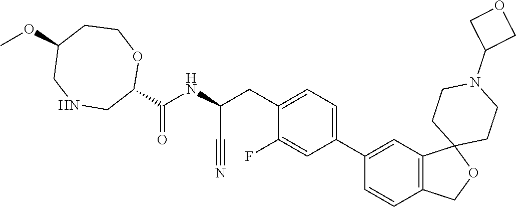 [SMILES: CO[C@H]1CCO[C@H](C(=O)N[C@H](C#N)Cc2ccc(-c3ccc4c(c3)C3(CCN(C5COC5)CC3)OC4)cc2F)CNC1] |
| F6 | 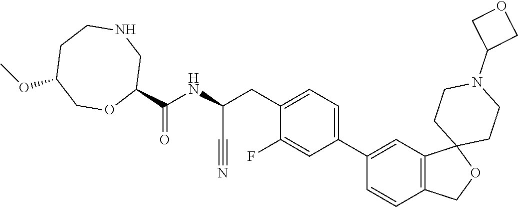 [SMILES: CO[C@@H]1CCNC[C@@H](C(=O)N[C@H](C#N)Cc2ccc(-c3ccc4c(c3)C3(CCN(C5COC5)CC3)OC4)cc2F)OC1] |
| F7 | 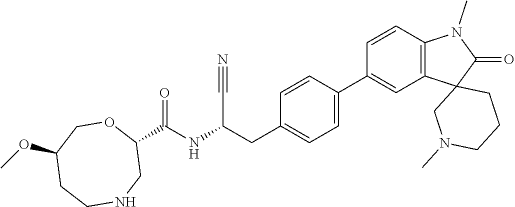 [SMILES: CO[C@@H]1CCNC[C@@H](C(=O)N[C@H](C#N)Cc2ccc(-c3ccc4c(c3)C3(CCCN(C)C3)C(=O)N4C)cc2)OC1] |
| F8 | 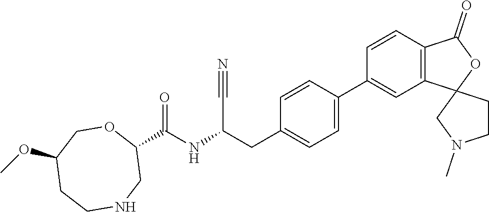 [SMILES: CO[C@@H]1CCNC[C@@H](C(=O)N[C@H](C#N)Cc2ccc(-c3ccc4c(c3)C3(CCN(C)C3)OC4=O)cc2)OC1] |
| F9 | 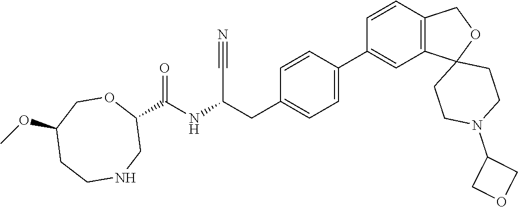 [SMILES: CO[C@@H]1CCNC[C@@H](C(=O)N[C@H](C#N)Cc2ccc(-c3ccc4c(c3)C3(CCN(C5COC5)CC3)OC4)cc2)OC1] |
| F10 | 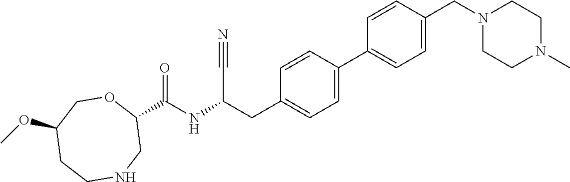 |
| F11 | 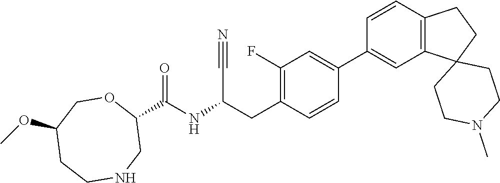 [SMILES: CO[C@@H]1CCNC[C@@H](C(=O)N[C@H](C#N)Cc2ccc(-c3ccc4c(c3)C3(CC4)CCN(C)CC3)cc2F)OC1] |
| F12 | 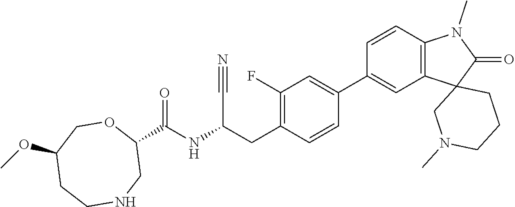 [SMILES: CO[C@@H]1CCNC[C@@H](C(=O)N[C@H](C#N)Cc2ccc(-c3ccc4c(c3)C3(CCCN(C)C3)C(=O)N4C)cc2F)OC1] |
| F13 | 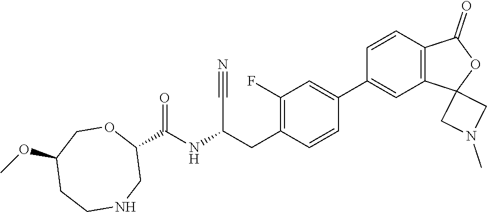 [SMILES: CO[C@@H]1CCNC[C@@H](C(=O)N[C@H](C#N)Cc2ccc(-c3ccc4c(c3)C3(CN(C)C3)OC4=O)cc2F)OC1] |
| F14 | 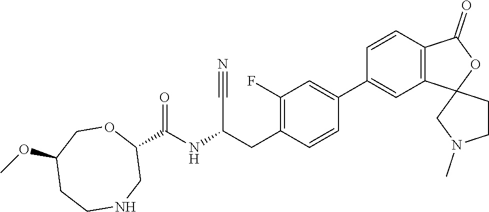 [SMILES: CO[C@@H]1CCNC[C@@H](C(=O)N[C@H](C#N)Cc2ccc(-c3ccc4c(c3)C3(CCN(C)C3)OC4=O)cc2F)OC1] |
| F15 | 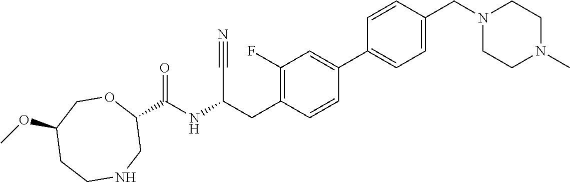 |
| F16 | 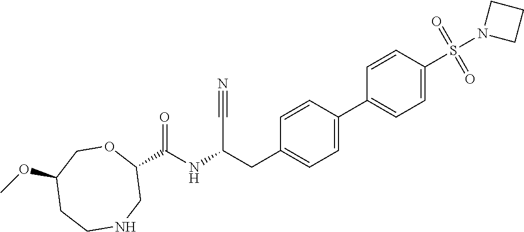 [SMILES: CO[C@@H]1CCNC[C@@H](C(=O)N[C@H](C#N)Cc2ccc(-c3ccc(S(=O)(=O)N4CCC4)cc3)cc2)OC1] |
| F17 | 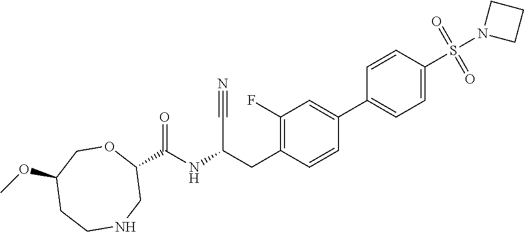 [SMILES: CO[C@@H]1CCNC[C@@H](C(=O)N[C@H](C#N)Cc2ccc(-c3ccc(S(=O)(=O)N4CCC4)cc3)cc2F)OC1] |
| F18 | 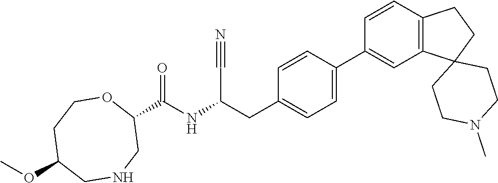 [SMILES: CO[C@H]1CCO[C@H](C(=O)N[C@H](C#N)Cc2ccc(-c3ccc4c(c3)C3(CC4)CCN(C)CC3)cc2)CNC1] |
| F19 | 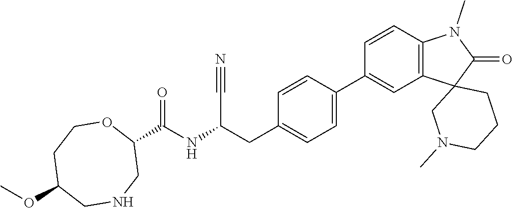 [SMILES: CO[C@H]1CCO[C@H](C(=O)N[C@H](C#N)Cc2ccc(-c3ccc4c(c3)C3(CCCN(C)C3)C(=O)N4C)cc2)CNC1] |
| F20 | 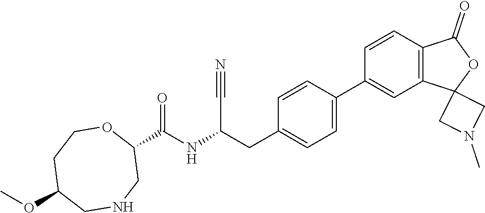 [SMILES: CO[C@H]1CCO[C@H](C(=O)N[C@H](C#N)Cc2ccc(-c3ccc4c(c3)C3(CN(C)C3)OC4=O)cc2)CNC1] |
| F21 | 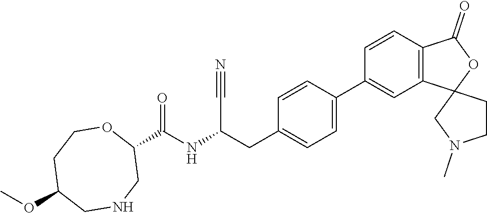 [SMILES: CO[C@H]1CCO[C@H](C(=O)N[C@H](C#N)Cc2ccc(-c3ccc4c(c3)C3(CCN(C)C3)OC4=O)cc2)CNC1] |
| F22 | 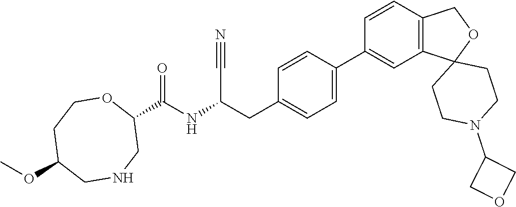 [SMILES: CO[C@H]1CCO[C@H](C(=O)N[C@H](C#N)Cc2ccc(-c3ccc4c(c3)C3(CCN(C5COC5)CC3)OC4)cc2)CNC1] |
| F23 | 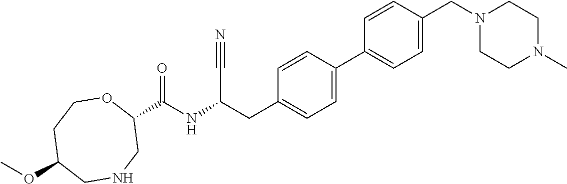 |
| F24 | 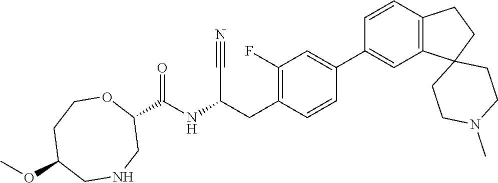 [SMILES: CO[C@H]1CCO[C@H](C(=O)N[C@H](C#N)Cc2ccc(-c3ccc4c(c3)C3(CC4)CCN(C)CC3)cc2F)CNC1] |
| F25 | 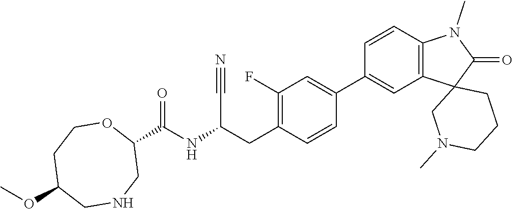 [SMILES: CO[C@H]1CCO[C@H](C(=O)N[C@H](C#N)Cc2ccc(-c3ccc4c(c3)C3(CCCN(C)C3)C(=O)N4C)cc2F)CNC1] |
| F26 | 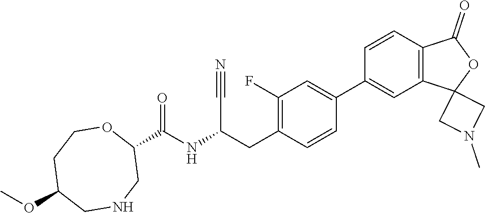 [SMILES: CO[C@H]1CCO[C@H](C(=O)N[C@H](C#N)Cc2ccc(-c3ccc4c(c3)C3(CN(C)C3)OC4=O)cc2F)CNC1] |
| F27 | 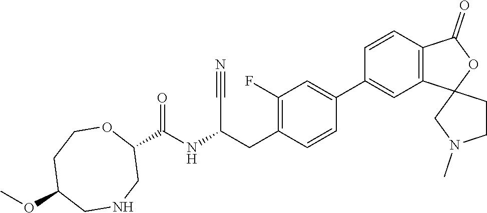 [SMILES: CO[C@H]1CCO[C@H](C(=O)N[C@H](C#N)Cc2ccc(-c3ccc4c(c3)C3(CCN(C)C3)OC4=O)cc2F)CNC1] |
| F28 | 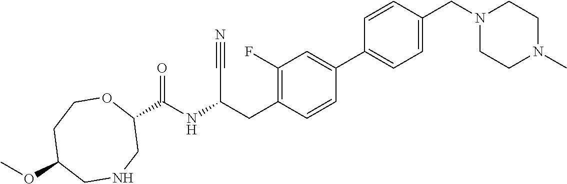 |
| F29 | 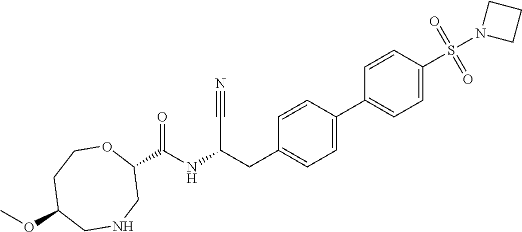 [SMILES: CO[C@H]1CCO[C@H](C(=O)N[C@H](C#N)Cc2ccc(-c3ccc(S(=O)(=O)N4CCC4)cc3)cc2)CNC1] |
| F30 | 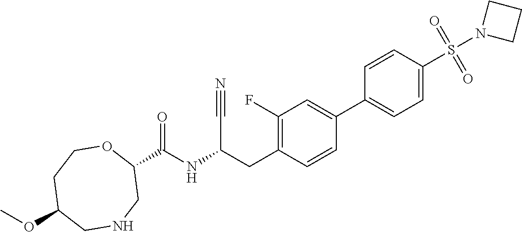 [SMILES: CO[C@H]1CCO[C@H](C(=O)N[C@H](C#N)Cc2ccc(-c3ccc(S(=O)(=O)N4CCC4)cc3)cc2F)CNC1] |

In embodiments, provided herein is a compound of formula (VII) selected from Compounds 32 or G1-G12, or a pharmaceutically acceptable salt thereof, a deuterated form thereof, racemic form thereof, or stereoisomer thereof.

In embodiments, the compound provided herein is Compound 32, or a pharmaceutically acceptable salt, a stereoisomer, or a deuterated form thereof.

In embodiments, the compound provided herein is Compound G2, or a pharmaceutically acceptable salt, a stereoisomer, or a deuterated form thereof.

In embodiments, the compound provided herein is Compound G3, or a pharmaceutically acceptable salt, a stereoisomer, or a deuterated form thereof.

In embodiments, the compound provided herein is Compound G9, or a pharmaceutically acceptable salt, a stereoisomer, or a deuterated form thereof.

In embodiments, the compound provided herein is Compound G10, or a pharmaceutically acceptable salt, a stereoisomer, or a deuterated form thereof.

In embodiments, the compound provided herein is Compound G12, or a pharmaceutically acceptable salt, a stereoisomer, or a deuterated form thereof.

In embodiments, provided herein is a compound in Table G, or a pharmaceutically acceptable salt thereof, deuterated form thereof, racemic form thereof, or stereoisomer thereof.

In embodiments, provided herein is a compound in Table G, or a pharmaceutically acceptable salt thereof, or stereoisomer thereof.

In embodiments, provided herein is a compound in Table G, or a pharmaceutically acceptable salt thereof.

In one embodiment, provided herein is a compound set forth in Table G.

In some embodiments, provided herein is a pharmaceutically acceptable salt of a compound in Table G.

TABLE G
Various compounds of the invention

| Compound No. | Structure |
|---|---|
| 32 | 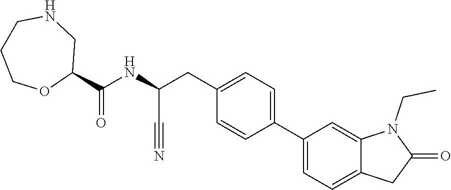 [SMILES: CCN1C(=O)Cc2ccc(-c3ccc(C[C@@H](C#N)NC(=O)[C@@H]4CNCCCO4)cc3)cc21] |
| G1 | 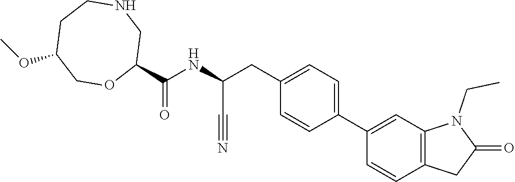 [SMILES: CCN1C(=O)Cc2ccc(-c3ccc(C[C@@H](C#N)NC(=O)[C@@H]4CNCC[C@@H](OC)CO4)cc3)cc21] |
| G2 | 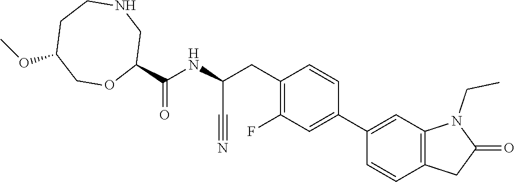 [SMILES: CCN1C(=O)Cc2ccc(-c3ccc(C[C@@H](C#N)NC(=O)[C@@H]4CNCC[C@@H](OC)CO4)c(F)c3)cc21] |
| G3 | 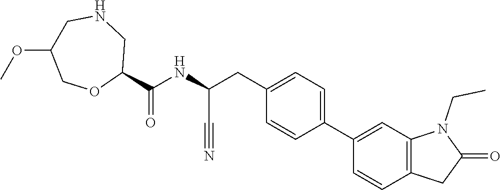 [SMILES: CCN1C(=O)Cc2ccc(-c3ccc(C[C@@H](C#N)NC(=O)[C@@H]4CNCC(OC)CO4)cc3)cc21] |
| G4 | 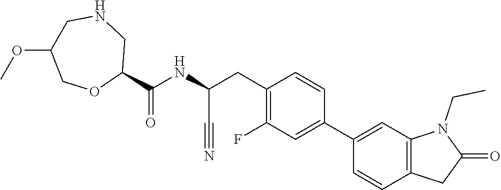 [SMILES: CCN1C(=O)Cc2ccc(-c3ccc(C[C@@H](C#N)NC(=O)[C@@H]4CNCC(OC)CO4)c(F)c3)cc21] |
| G5 | 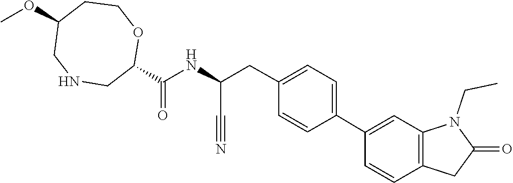 [SMILES: CCN1C(=O)Cc2ccc(-c3ccc(C[C@@H](C#N)NC(=O)[C@@H]4CNC[C@@H](OC)CCO4)cc3)cc21] |
| G6 | 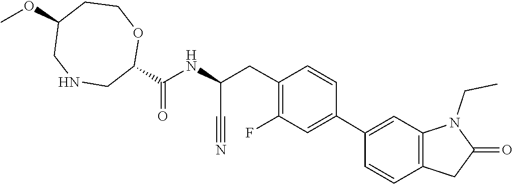 [SMILES: CCN1C(=O)Cc2ccc(-c3ccc(C[C@@H](C#N)NC(=O)[C@@H]4CNC[C@@H](OC)CCO4)c(F)c3)cc21] |
| G7 | 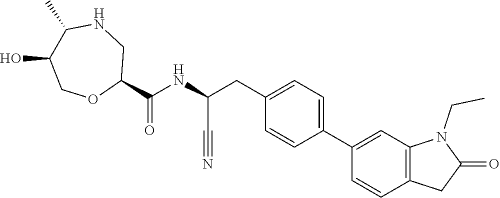 [SMILES: CCN1C(=O)Cc2ccc(-c3ccc(C[C@@H](C#N)NC(=O)[C@@H]4CN[C@@H](C)[C@H](O)CO4)cc3)cc21] |
| G8 | 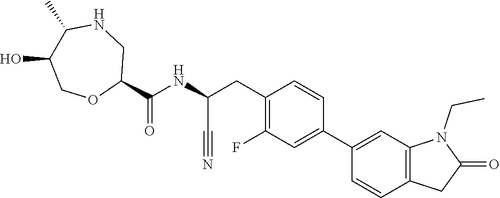 [SMILES: CCN1C(=O)Cc2ccc(-c3ccc(C[C@@H](C#N)NC(=O)[C@@H]4CN[C@@H](C)[C@H](O)CO4)c(F)c3)cc21] |
| G9 | 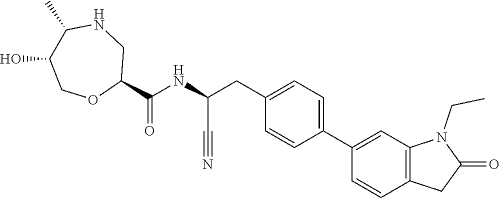 [SMILES: CCN1C(=O)Cc2ccc(-c3ccc(C[C@@H](C#N)NC(=O)[C@@H]4CN[C@@H](C)[C@@H](O)CO4)cc3)cc21] |
| G10 | 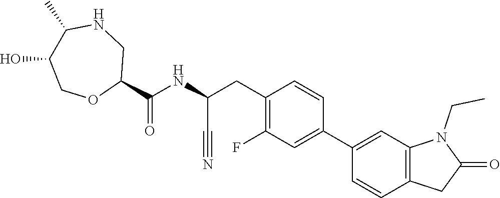 [SMILES: CCN1C(=O)Cc2ccc(-c3ccc(C[C@@H](C#N)NC(=O)[C@@H]4CN[C@@H](C)[C@@H](O)CO4)c(F)c3)cc21] |
| G11 | 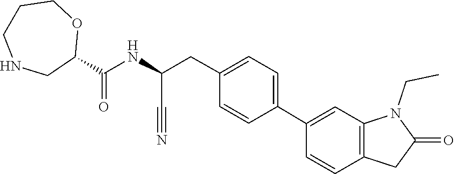 [SMILES: CCN1C(=O)Cc2ccc(-c3ccc(C[C@@H](C#N)NC(=O)[C@@H]4CNCCCO4)cc3)cc21] |
| G12 | 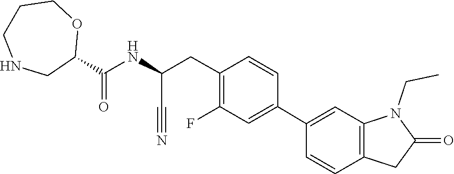 [SMILES: CCN1C(=O)Cc2ccc(-c3ccc(C[C@@H](C#N)NC(=O)[C@@H]4CNCCCO4)c(F)c3)cc21] |

In embodiments, provided herein is a compound of formula (VIII) selected from Compounds H1-H44, or a pharmaceutically acceptable salt thereof, a deuterated form thereof, racemic form thereof, or stereoisomer thereof.

In embodiments, the compound provided herein is Compound H5, or a pharmaceutically acceptable salt, a stereoisomer, or a deuterated form thereof.

In embodiments, the compound provided herein is Compound H7, or a pharmaceutically acceptable salt, a stereoisomer, or a deuterated form thereof.

In embodiments, the compound provided herein is Compound H12, or a pharmaceutically acceptable salt, a stereoisomer, or a deuterated form thereof.

In embodiments, the compound provided herein is Compound H29, or a pharmaceutically acceptable salt, a stereoisomer, or a deuterated form thereof.

In embodiments, the compound provided herein is Compound H31, or a pharmaceutically acceptable salt, a stereoisomer, or a deuterated form thereof.

In embodiments, the compound provided herein is Compound H36, or a pharmaceutically acceptable salt, a stereoisomer, or a deuterated form thereof.

In embodiments, provided herein is a compound in Table H, or a pharmaceutically acceptable salt thereof, deuterated form thereof, racemic form thereof, or stereoisomer thereof.

In embodiments, provided herein is a compound in Table H, or a pharmaceutically acceptable salt thereof, or stereoisomer thereof.

In embodiments, provided herein is a compound in Table H, or a pharmaceutically acceptable salt thereof.

In one embodiment, provided herein is a compound set forth in Table H.

In some embodiments, provided herein is a pharmaceutically acceptable salt of a compound in Table H.

TABLE H
Various compounds of the invention

| Compound No. | Structure |
|---|---|
| H1 | 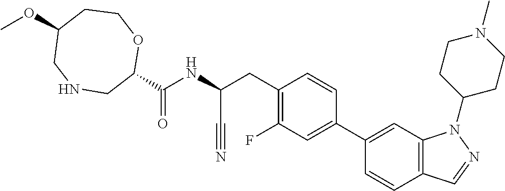 [SMILES: CO[C@H]1CCO[C@H](C(=O)N[C@H](C#N)Cc2ccc(-c3ccc4cnn(C5CCN(C)CC5)c4c3)cc2F)CNC1] |
| H2 | 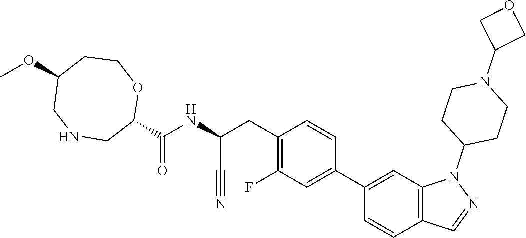 [SMILES: CO[C@H]1CCO[C@H](C(=O)N[C@H](C#N)Cc2ccc(-c3ccc4cnn(C5CCN(C6COC6)CC5)c4c3)cc2F)CNC1] |
| H3 | 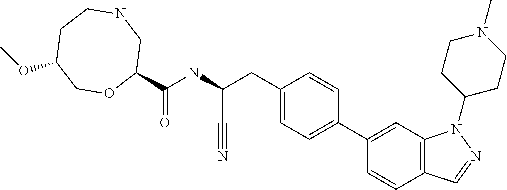 [SMILES: CO[C@@H]1CCNC[C@@H](C(=O)N[C@H](C#N)Cc2ccc(-c3ccc4cnn(C5CCN(C)CC5)c4c3)cc2)OC1] |
| H4 | 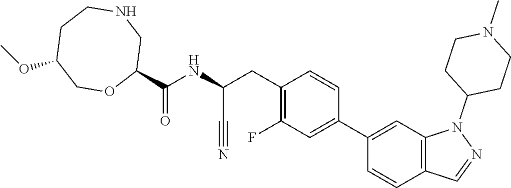 [SMILES: CO[C@@H]1CCNC[C@@H](C(=O)N[C@H](C#N)Cc2ccc(-c3ccc4cnn(C5CCN(C)CC5)c4c3)cc2F)OC1] |
| H5 | 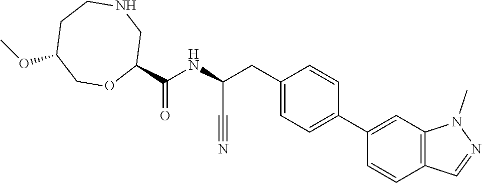 [SMILES: CO[C@@H]1CCNC[C@@H](C(=O)N[C@H](C#N)Cc2ccc(-c3ccc4cnn(C)c4c3)cc2)OC1] |
| H6 | 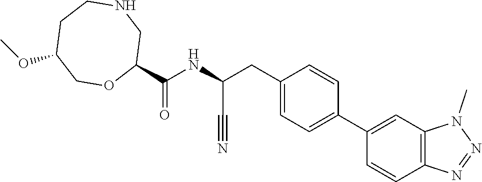 [SMILES: CO[C@@H]1CCNC[C@@H](C(=O)N[C@H](C#N)Cc2ccc(-c3ccc4nnn(C)c4c3)cc2)OC1] |
| H7 | 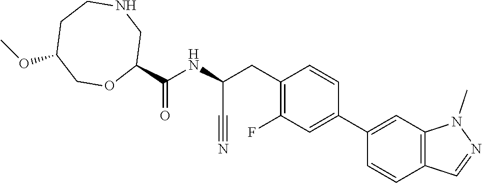 [SMILES: CO[C@@H]1CCNC[C@@H](C(=O)N[C@H](C#N)Cc2ccc(-c3ccc4cnn(C)c4c3)cc2F)OC1] |
| H8 | 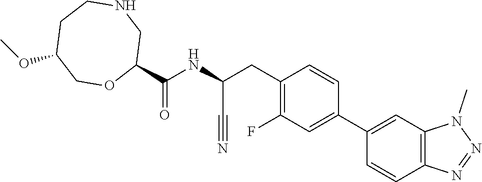 [SMILES: CO[C@@H]1CCNC[C@@H](C(=O)N[C@H](C#N)Cc2ccc(-c3ccc4nnn(C)c4c3)cc2F)OC1] |
| H9 | 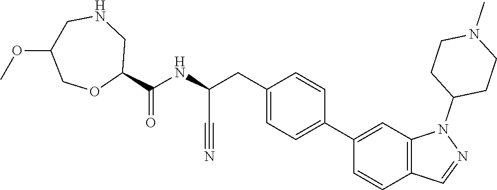 [SMILES: COC1CNC[C@@H](C(=O)N[C@H](C#N)Cc2ccc(-c3ccc4cnn(C5CCN(C)CC5)c4c3)cc2)OC1] |
| H10 | 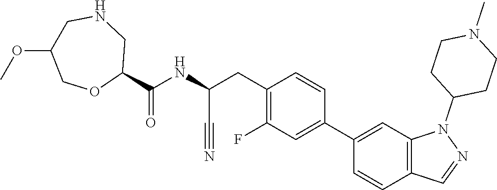 [SMILES: COC1CNC[C@@H](C(=O)N[C@H](C#N)Cc2ccc(-c3ccc4cnn(C5CCN(C)CC5)c4c3)cc2F)OC1] |
| H11 | 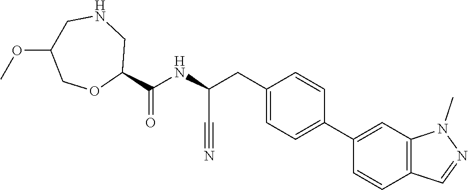 [SMILES: COC1CNC[C@@H](C(=O)N[C@H](C#N)Cc2ccc(-c3ccc4cnn(C)c4c3)cc2)OC1] |
| H12 | 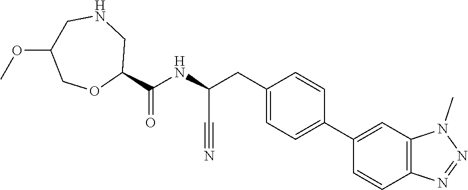 [SMILES: COC1CNC[C@@H](C(=O)N[C@H](C#N)Cc2ccc(-c3ccc4nnn(C)c4c3)cc2)OC1] |
| H12-A | 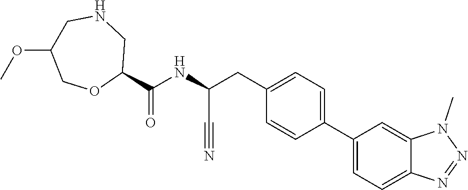 [SMILES: COC1CNC[C@@H](C(=O)N[C@H](C#N)Cc2ccc(-c3ccc4nnn(C)c4c3)cc2)OC1] |
| H12-B | 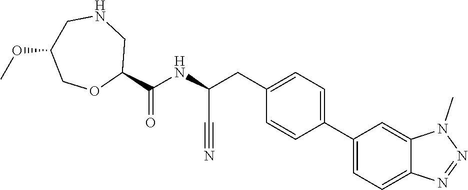 [SMILES: CO[C@@H]1CNC[C@@H](C(=O)N[C@H](C#N)Cc2ccc(-c3ccc4nnn(C)c4c3)cc2)OC1] |
| H13 | 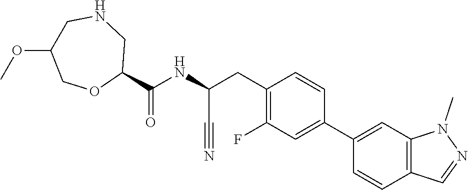 [SMILES: COC1CNC[C@@H](C(=O)N[C@H](C#N)Cc2ccc(-c3ccc4cnn(C)c4c3)cc2F)OC1] |
| H14 | 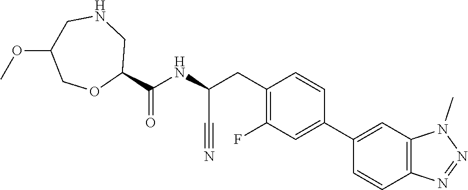 [SMILES: COC1CNC[C@@H](C(=O)N[C@H](C#N)Cc2ccc(-c3ccc4nnn(C)c4c3)cc2F)OC1] |
| H15 | 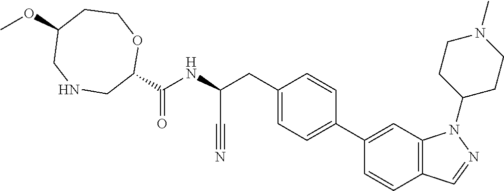 [SMILES: CO[C@H]1CCO[C@H](C(=O)N[C@H](C#N)Cc2ccc(-c3ccc4cnn(C5CCN(C)CC5)c4c3)cc2)CNC1] |
| H16 | 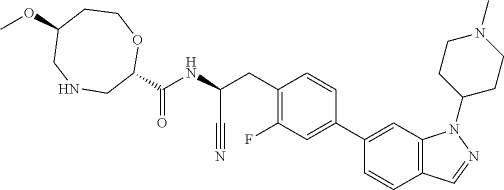 [SMILES: CO[C@H]1CCO[C@H](C(=O)N[C@H](C#N)Cc2ccc(-c3ccc4cnn(C5CCN(C)CC5)c4c3)cc2F)CNC1] |
| H17 | 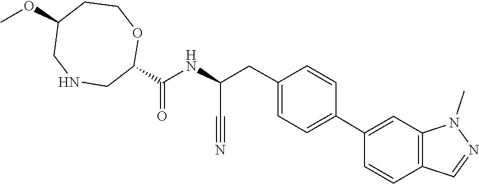 [SMILES: CO[C@H]1CCO[C@H](C(=O)N[C@H](C#N)Cc2ccc(-c3ccc4cnn(C)c4c3)cc2)CNC1] |
| H18 | 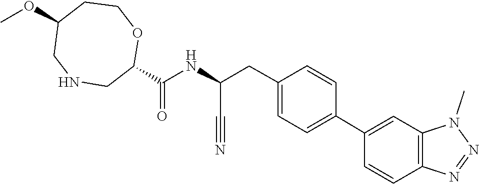 [SMILES: CO[C@H]1CCO[C@H](C(=O)N[C@H](C#N)Cc2ccc(-c3ccc4nnn(C)c4c3)cc2)CNC1] |
| H19 | 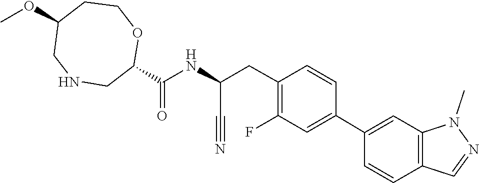 [SMILES: CO[C@H]1CCO[C@H](C(=O)N[C@H](C#N)Cc2ccc(-c3ccc4cnn(C)c4c3)cc2F)CNC1] |
| H20 | 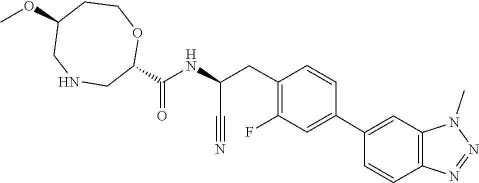 [SMILES: CO[C@H]1CCO[C@H](C(=O)N[C@H](C#N)Cc2ccc(-c3ccc4nnn(C)c4c3)cc2F)CNC1] |
| H21 | 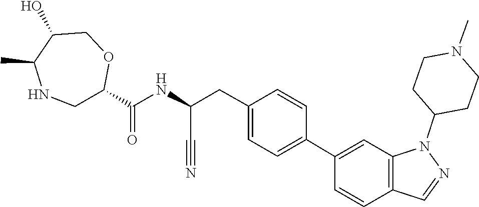 [SMILES: C[C@@H]1NC[C@@H](C(=O)N[C@H](C#N)Cc2ccc(-c3ccc4cnn(C5CCN(C)CC5)c4c3)cc2)OC[C@H]1O] |
| H22 | 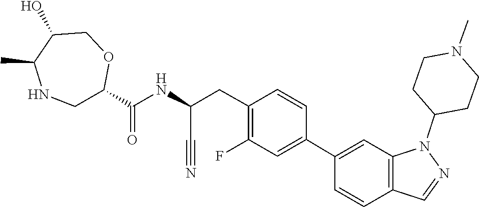 [SMILES: C[C@@H]1NC[C@@H](C(=O)N[C@H](C#N)Cc2ccc(-c3ccc4cnn(C5CCN(C)CC5)c4c3)cc2F)OC[C@H]1O] |
| H23 | 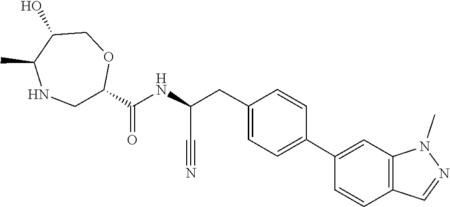 [SMILES: C[C@@H]1NC[C@@H](C(=O)N[C@H](C#N)Cc2ccc(-c3ccc4cnn(C)c4c3)cc2)OC[C@H]1O] |
| H24 | 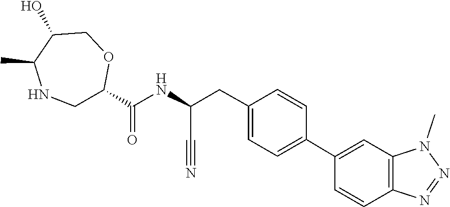 [SMILES: C[C@@H]1NC[C@@H](C(=O)N[C@H](C#N)Cc2ccc(-c3ccc4nnn(C)c4c3)cc2)OC[C@H]1O] |
| H25 | 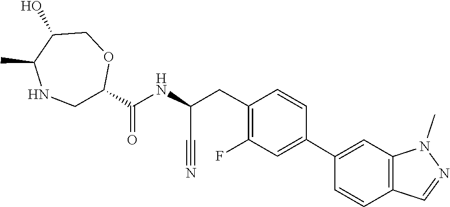 [SMILES: C[C@@H]1NC[C@@H](C(=O)N[C@H](C#N)Cc2ccc(-c3ccc4cnn(C)c4c3)cc2F)OC[C@H]1O] |
| H26 | 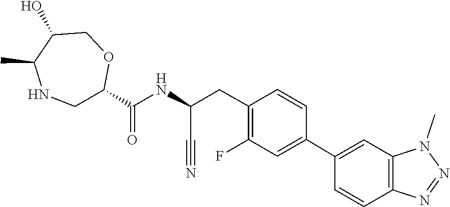 [SMILES: C[C@@H]1NC[C@@H](C(=O)N[C@H](C#N)Cc2ccc(-c3ccc4nnn(C)c4c3)cc2F)OC[C@H]1O] |
| H27 | 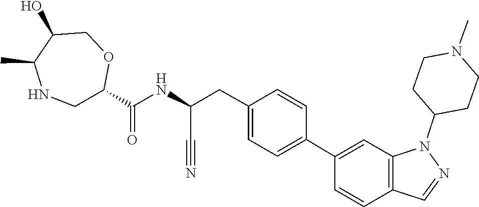 [SMILES: C[C@@H]1NC[C@@H](C(=O)N[C@H](C#N)Cc2ccc(-c3ccc4cnn(C5CCN(C)CC5)c4c3)cc2)OC[C@@H]1O] |
| H28 | 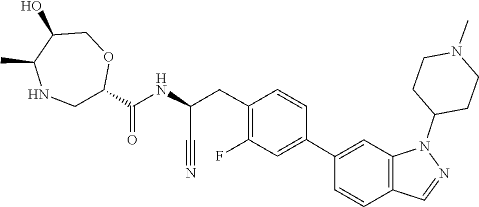 [SMILES: C[C@@H]1NC[C@@H](C(=O)N[C@H](C#N)Cc2ccc(-c3ccc4cnn(C5CCN(C)CC5)c4c3)cc2F)OC[C@@H]1O] |
| H29 | 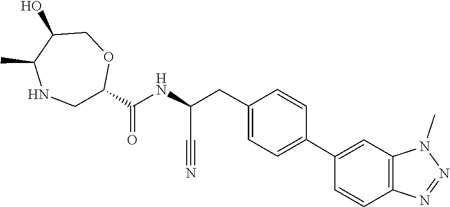 [SMILES: C[C@@H]1NC[C@@H](C(=O)N[C@H](C#N)Cc2ccc(-c3ccc4nnn(C)c4c3)cc2)OC[C@@H]1O] |
| H30 | 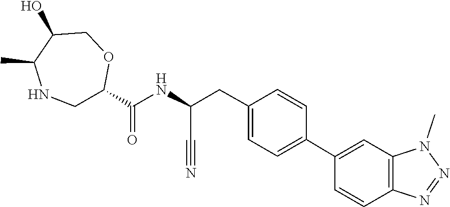 [SMILES: C[C@@H]1NC[C@@H](C(=O)N[C@H](C#N)Cc2ccc(-c3ccc4nnn(C)c4c3)cc2)OC[C@@H]1O] |
| H31 | 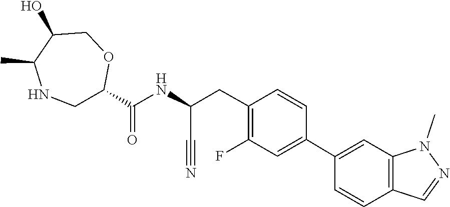 [SMILES: C[C@@H]1NC[C@@H](C(=O)N[C@H](C#N)Cc2ccc(-c3ccc4cnn(C)c4c3)cc2F)OC[C@@H]1O] |
| H32 | 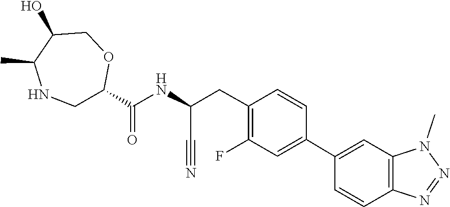 [SMILES: C[C@@H]1NC[C@@H](C(=O)N[C@H](C#N)Cc2ccc(-c3ccc4nnn(C)c4c3)cc2F)OC[C@@H]1O] |
| H33 | 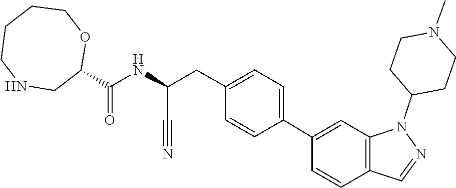 [SMILES: CN1CCC(n2ncc3ccc(-c4ccc(C[C@@H](C#N)NC(=O)[C@@H]5CNCCCCO5)cc4)cc32)CC1] |
| H34 | 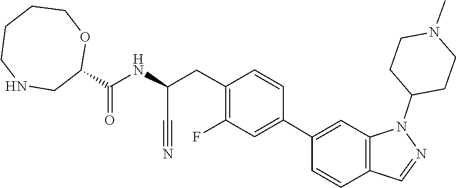 [SMILES: CN1CCC(n2ncc3ccc(-c4ccc(C[C@@H](C#N)NC(=O)[C@@H]5CNCCCCO5)c(F)c4)cc32)CC1] |
| H35 | 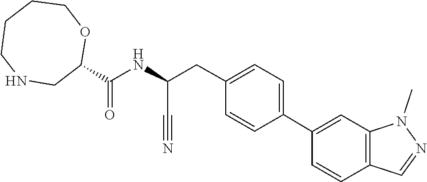 [SMILES: Cn1ncc2ccc(-c3ccc(C[C@@H](C#N)NC(=O)[C@@H]4CNCCCCO4)cc3)cc21] |
| H36 | 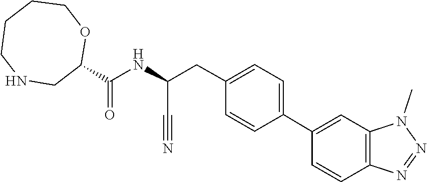 [SMILES: Cn1nnc2ccc(-c3ccc(C[C@@H](C#N)NC(=O)[C@@H]4CNCCCCO4)cc3)cc21] |
| H37 | 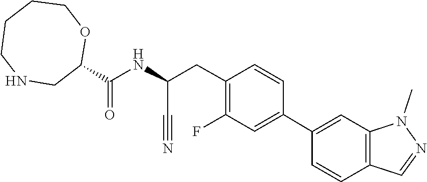 [SMILES: Cn1ncc2ccc(-c3ccc(C[C@@H](C#N)NC(=O)[C@@H]4CNCCCCO4)c(F)c3)cc21] |
| H38 | 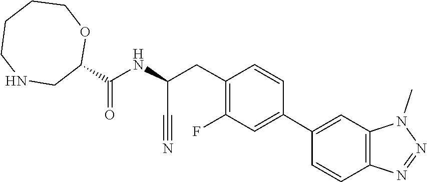 [SMILES: Cn1nnc2ccc(-c3ccc(C[C@@H](C#N)NC(=O)[C@@H]4CNCCCCO4)c(F)c3)cc21] |
| H39 | 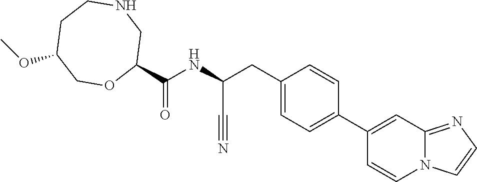 [SMILES: CO[C@@H]1CCNC[C@@H](C(=O)N[C@H](C#N)Cc2ccc(-c3ccn4ccnc4c3)cc2)OC1] |
| H40 | 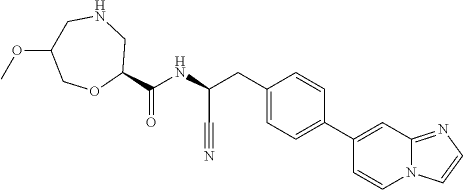 [SMILES: COC1CNC[C@@H](C(=O)N[C@H](C#N)Cc2ccc(-c3ccn4ccnc4c3)cc2)OC1] |
| H41 | 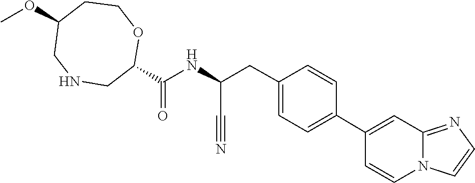 [SMILES: CO[C@H]1CCO[C@H](C(=O)N[C@H](C#N)Cc2ccc(-c3ccn4ccnc4c3)cc2)CNC1] |
| H42 | 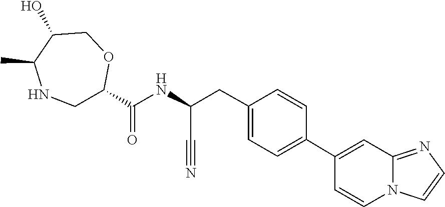 [SMILES: C[C@@H]1NC[C@@H](C(=O)N[C@H](C#N)Cc2ccc(-c3ccn4ccnc4c3)cc2)OC[C@H]1O] |
| H43 | 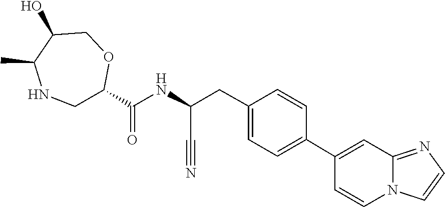 [SMILES: C[C@@H]1NC[C@@H](C(=O)N[C@H](C#N)Cc2ccc(-c3ccn4ccnc4c3)cc2)OC[C@@H]1O] |
| H44 | 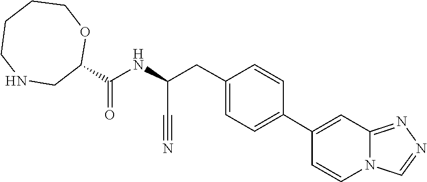 [SMILES: N#C[C@H](Cc1ccc(-c2ccn3cnnc3c2)cc1)NC(=O)[C@@H]1CNCCCCO1] |

In embodiments, provided herein is a compound of formula (IX) selected from Compounds I1-I20, or a pharmaceutically acceptable salt thereof, a deuterated form thereof, racemic form thereof, or stereoisomer thereof.

In embodiments, provided herein is a compound in Table I, or a pharmaceutically acceptable salt thereof, deuterated form thereof, racemic form thereof, or stereoisomer thereof.

In embodiments, provided herein is a compound in Table I, or a pharmaceutically acceptable salt thereof, or stereoisomer thereof.

In embodiments, provided herein is a compound in Table I, or a pharmaceutically acceptable salt thereof.

In one embodiment, provided herein is a compound set forth in Table I.

In some embodiments, provided herein is a pharmaceutically acceptable salt of a compound in Table I.

TABLE I
Various compounds of the invention

| Compound No. | Structure |
|---|---|
| I1 | 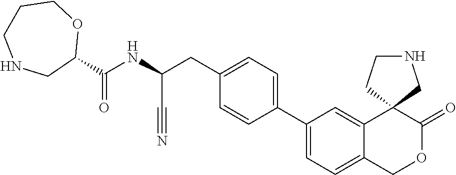 [SMILES: N#C[C@H](Cc1ccc(-c2ccc3c(c2)[C@]2(CCNC2)C(=O)OC3)cc1)NC(=O)[C@@H]1CNCCCO1] |
| I2 | 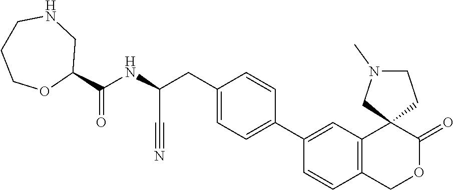 [SMILES: CN1CC[C@]2(C1)C(=O)OCc1ccc(-c3ccc(C[C@@H](C#N)NC(=O)[C@@H]4CNCCCO4)cc3)cc12] |
| I3 | 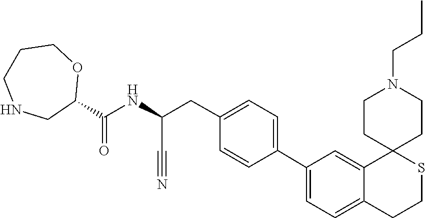 [SMILES: CCCN1CCC2(CC1)SCCc1ccc(-c3ccc(C[C@@H](C#N)NC(=O)[C@@H]4CNCCCO4)cc3)cc12] |
| I4 | 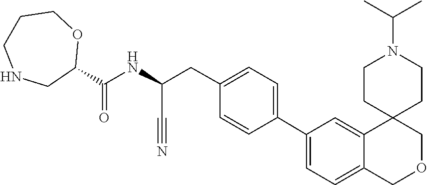 [SMILES: CC(C)N1CCC2(CC1)COCc1ccc(-c3ccc(C[C@@H](C#N)NC(=O)[C@@H]4CNCCCO4)cc3)cc12] |
| I5 | 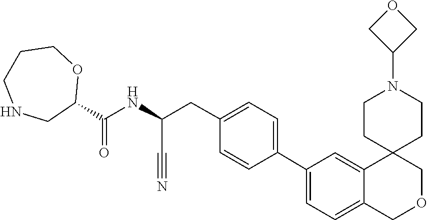 [SMILES: N#C[C@H](Cc1ccc(-c2ccc3c(c2)C2(CCN(C4COC4)CC2)COC3)cc1)NC(=O)[C@@H]1CNCCCO1] |
| I6 | 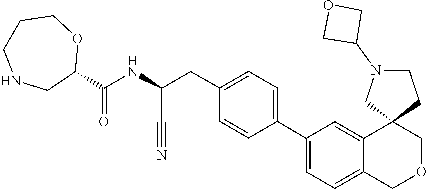 [SMILES: N#C[C@H](Cc1ccc(-c2ccc3c(c2)[C@@]2(CCN(C4COC4)C2)COC3)cc1)NC(=O)[C@@H]1CNCCCO1] |
| I7 | 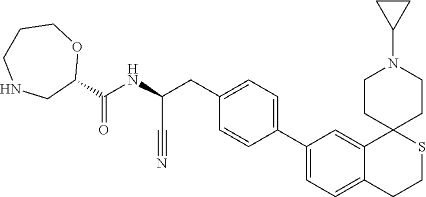 [SMILES: N#C[C@H](Cc1ccc(-c2ccc3c(c2)C2(CCN(C4CC4)CC2)SCC3)cc1)NC(=O)[C@@H]1CNCCCO1] |
| I8 | 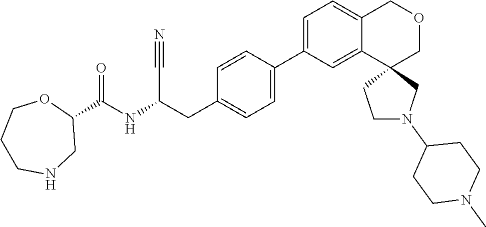 [SMILES: CN1CCC(N2CC[C@]3(COCc4ccc(-c5ccc(C[C@@H](C#N)NC(=O)[C@@H]6CNCCCO6)cc5)cc43)C2)CC1] |
| I9 | 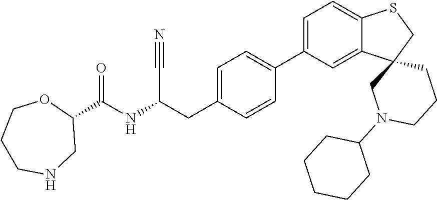 [SMILES: N#C[C@H](Cc1ccc(-c2ccc3c(c2)[C@@]2(CCCN(C4CCCCC4)C2)CS3)cc1)NC(=O)[C@@H]1CNCCCO1] |
| I10 | 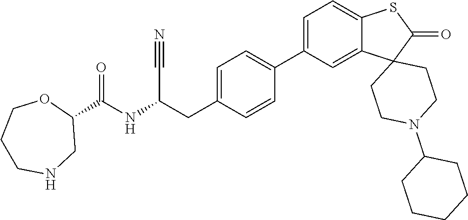 [SMILES: N#C[C@H](Cc1ccc(-c2ccc3c(c2)C2(CCN(C4CCCCC4)CC2)C(=O)S3)cc1)NC(=O)[C@@H]1CNCCCO1] |
| I11 | 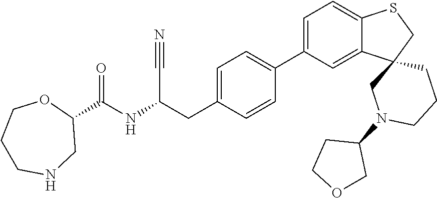 [SMILES: N#C[C@H](Cc1ccc(-c2ccc3c(c2)[C@@]2(CCCN([C@@H]4CCOC4)C2)CS3)cc1)NC(=O)[C@@H]1CNCCCO1] |
| I12 | 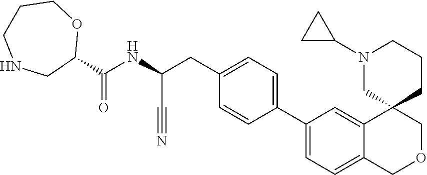 [SMILES: N#C[C@H](Cc1ccc(-c2ccc3c(c2)[C@@]2(CCCN(C4CC4)C2)COC3)cc1)NC(=O)[C@@H]1CNCCCO1] |
| I13 | 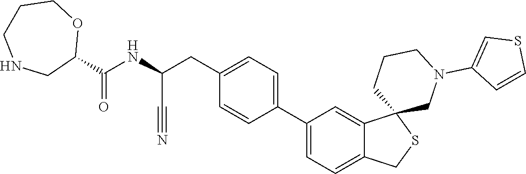 [SMILES: N#C[C@H](Cc1ccc(-c2ccc3c(c2)[C@]2(CCCN(c4ccsc4)C2)SC3)cc1)NC(=O)[C@@H]1CNCCCO1] |
| I14 | 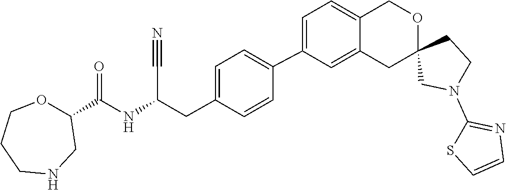 [SMILES: N#C[C@H](Cc1ccc(-c2ccc3c(c2)C[C@]2(CCN(c4nccs4)C2)OC3)cc1)NC(=O)[C@@H]1CNCCCO1] |
| I15 | 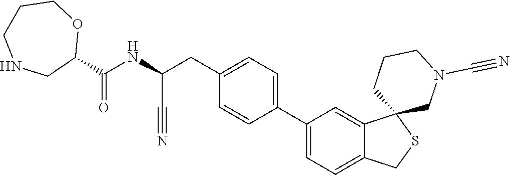 [SMILES: N#C[C@H](Cc1ccc(-c2ccc3c(c2)[C@]2(CCCN(C#N)C2)SC3)cc1)NC(=O)[C@@H]1CNCCCO1] |
| I16 | 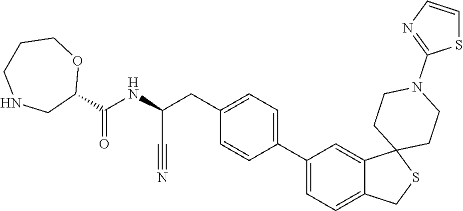 [SMILES: N#C[C@H](Cc1ccc(-c2ccc3c(c2)C2(CCN(c4nccs4)CC2)SC3)cc1)NC(=O)[C@@H]1CNCCCO1] |
| I17 | 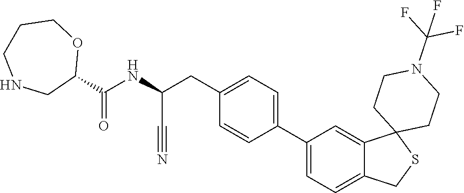 [SMILES: N#C[C@H](Cc1ccc(-c2ccc3c(c2)C2(CCN(C(F)(F)F)CC2)SC3)cc1)NC(=O)[C@@H]1CNCCCO1] |
| I18 | 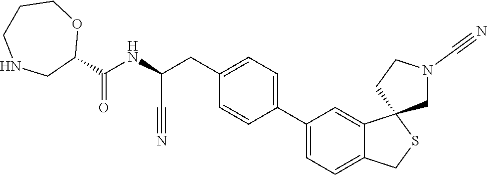 [SMILES: N#C[C@H](Cc1ccc(-c2ccc3c(c2)[C@]2(CCN(C#N)C2)SC3)cc1)NC(=O)[C@@H]1CNCCCO1] |
| I19 | 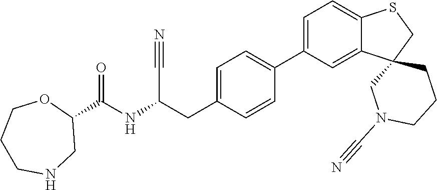 [SMILES: N#C[C@H](Cc1ccc(-c2ccc3c(c2)[C@]2(CCCN(C#N)C2)CS3)cc1)NC(=O)[C@@H]1CNCCCO1] |
| I20 | 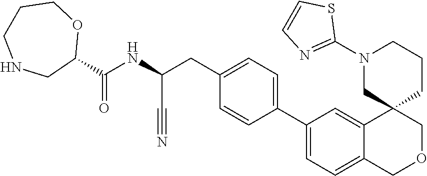 [SMILES: N#C[C@H](Cc1ccc(-c2ccc3c(c2)[C@]2(CCCN(c4nccs4)C2)COC3)cc1)NC(=O)[C@@H]1CNCCCO1] |

In embodiments, provided herein is a compound in Table J, or a pharmaceutically acceptable salt thereof, racemic form thereof, or stereoisomer thereof.

In embodiments, provided herein is a compound in Table J, or a pharmaceutically acceptable salt thereof, or stereoisomer thereof.

In embodiments, provided herein is a compound in Table J, or a pharmaceutically acceptable salt thereof.

In one embodiment, provided herein is a compound set forth in Table J.

In some embodiments, provided herein is a pharmaceutically acceptable salt of a compound in Table J.

In embodiments, provided herein is a compound selected from Compounds 1-5, 7-11, or 18-34, or a pharmaceutically acceptable salt thereof, a deuterated form thereof, racemic form thereof, or stereoisomer thereof. In embodiments, provided herein is a compound selected from Compounds 1-5, 7-11, or 18-35, or a pharmaceutically acceptable salt thereof, a deuterated form thereof, racemic form thereof, or stereoisomer thereof.

In embodiments, provided herein is a compound selected from Compounds 6 or 12-14, or a pharmaceutically acceptable salt thereof, or a deuterated form thereof.

In embodiments, the compound provided herein is Compound 32, or a pharmaceutically acceptable salt, a stereoisomer, or a deuterated form thereof.

Table J. Various compounds of the invention

TABLE J
Various compounds of the invention

| Compound No. | Structure |
|---|---|
| 1 | 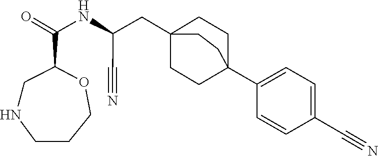 [SMILES: N#Cc1ccc(C23CCC(C[C@@H](C#N)NC(=O)[C@@H]4CNCCCO4)(CC2)CC3)cc1] |
| 2 | 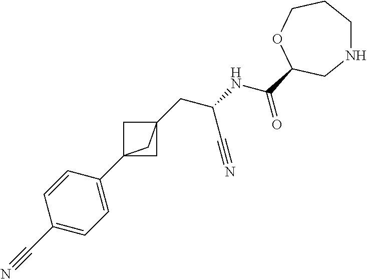 [SMILES: N#Cc1ccc(C23CC(C[C@@H](C#N)NC(=O)[C@@H]4CNCCCO4)(C2)C3)cc1] |
| 3 | 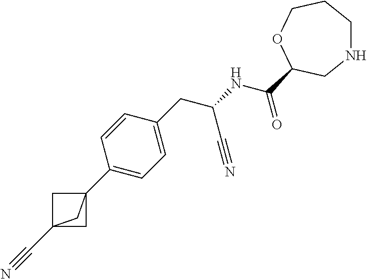 [SMILES: N#C[C@H](Cc1ccc(C23CC(C#N)(C2)C3)cc1)NC(=O)[C@@H]1CNCCCO1] |
| 4 | 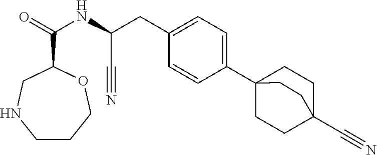 [SMILES: N#C[C@H](Cc1ccc(C23CCC(C#N)(CC2)CC3)cc1)NC(=O)[C@@H]1CNCCCO1] |
| 5 | 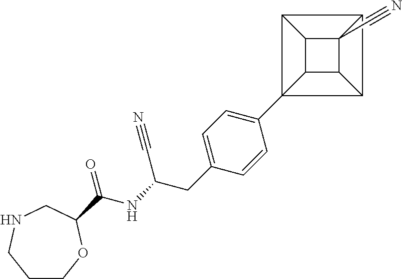 [SMILES: N#C[C@H](Cc1ccc(C23C4C5C2C2C3C4C52C#N)cc1)NC(=O)[C@@H]1CNCCCO1] |
| 6 | 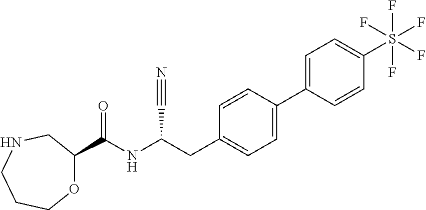 [SMILES: N#C[C@H](Cc1ccc(-c2ccc(S(F)(F)(F)(F)F)cc2)cc1)NC(=O)[C@@H]1CNCCCO1] |
| 7 | 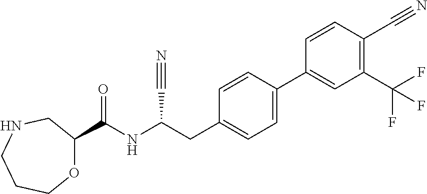 [SMILES: N#Cc1ccc(-c2ccc(C[C@@H](C#N)NC(=O)[C@@H]3CNCCCO3)cc2)cc1C(F)(F)F] |
| 8 | 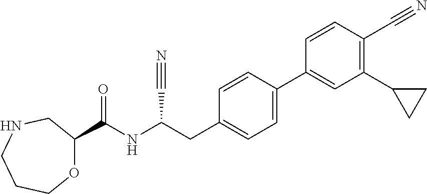 [SMILES: N#Cc1ccc(-c2ccc(C[C@@H](C#N)NC(=O)[C@@H]3CNCCCO3)cc2)cc1C1CC1] |
| 9 | 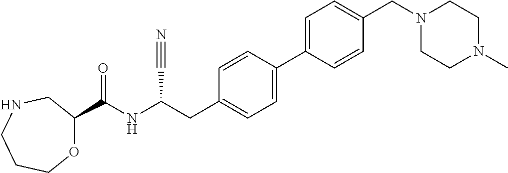 [SMILES: CN1CCN(Cc2ccc(-c3ccc(C[C@@H](C#N)NC(=O)[C@@H]4CNCCCO4)cc3)cc2)CC1] |
| 10 | 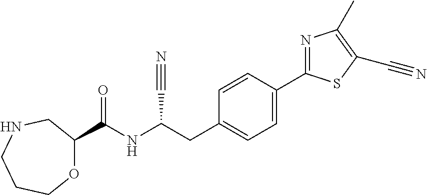 [SMILES: Cc1nc(-c2ccc(C[C@@H](C#N)NC(=O)[C@@H]3CNCCCO3)cc2)sc1C#N] |
| 11 | 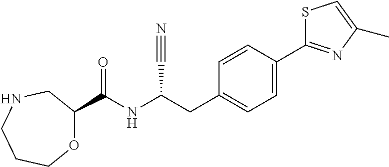 [SMILES: Cc1csc(-c2ccc(C[C@@H](C#N)NC(=O)[C@@H]3CNCCCO3)cc2)n1] |
| 12 | 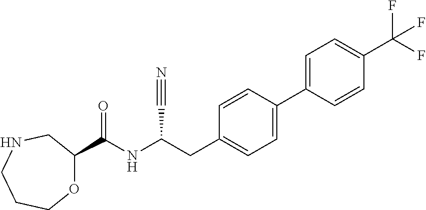 [SMILES: N#C[C@H](Cc1ccc(-c2ccc(C(F)(F)F)cc2)cc1)NC(=O)[C@@H]1CNCCCO1] |
| 13 | 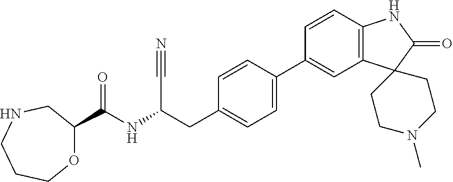 [SMILES: CN1CCC2(CC1)C(=O)Nc1ccc(-c3ccc(C[C@@H](C#N)NC(=O)[C@@H]4CNCCCO4)cc3)cc12] |
| 14 | 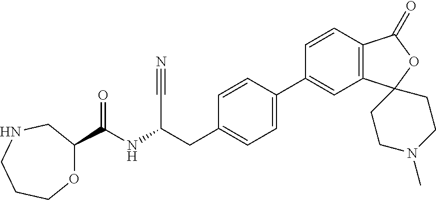 [SMILES: CN1CCC2(CC1)OC(=O)c1ccc(-c3ccc(C[C@@H](C#N)NC(=O)[C@@H]4CNCCCO4)cc3)cc12] |
| 18 | 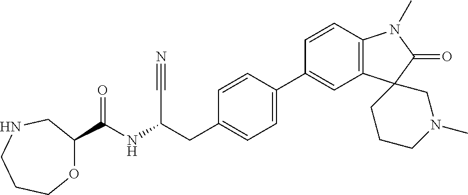 [SMILES: CN1CCCC2(C1)C(=O)N(C)c1ccc(-c3ccc(C[C@@H](C#N)NC(=O)[C@@H]4CNCCCO4)cc3)cc12] |
| 19 | 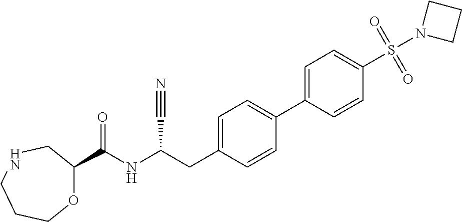 [SMILES: N#C[C@H](Cc1ccc(-c2ccc(S(=O)(=O)N3CCC3)cc2)cc1)NC(=O)[C@@H]1CNCCCO1] |
| 20 | 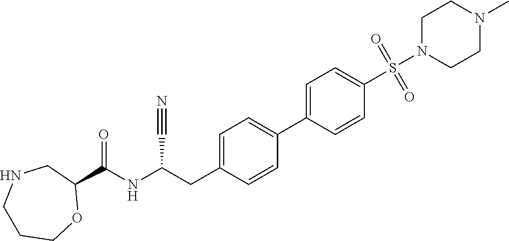 [SMILES: CN1CCN(S(=O)(=O)c2ccc(-c3ccc(C[C@@H](C#N)NC(=O)[C@@H]4CNCCCO4)cc3)cc2)CC1] |
| 21 | 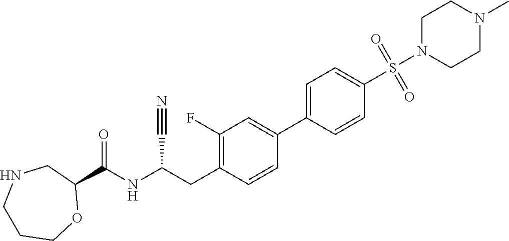 [SMILES: CN1CCN(S(=O)(=O)c2ccc(-c3ccc(C[C@@H](C#N)NC(=O)[C@@H]4CNCCCO4)c(F)c3)cc2)CC1] |
| 22 | 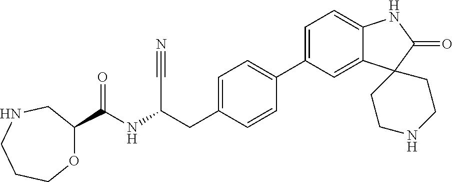 [SMILES: N#C[C@H](Cc1ccc(-c2ccc3c(c2)C2(CCNCC2)C(=O)N3)cc1)NC(=O)[C@@H]1CNCCCO1] |
| 23 | 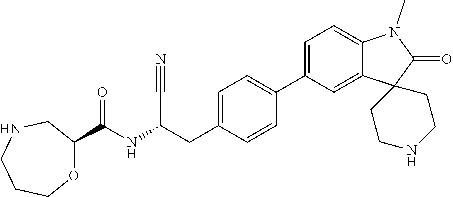 [SMILES: CN1C(=O)C2(CCNCC2)c2cc(-c3ccc(C[C@@H](C#N)NC(=O)[C@@H]4CNCCCO4)cc3)ccc21] |
| 24 | 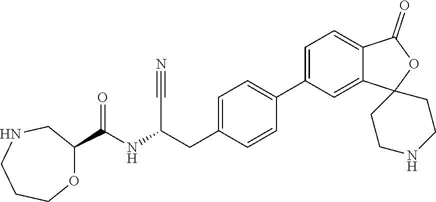 [SMILES: N#C[C@H](Cc1ccc(-c2ccc3c(c2)C2(CCNCC2)OC3=O)cc1)NC(=O)[C@@H]1CNCCCO1] |
| 25 | 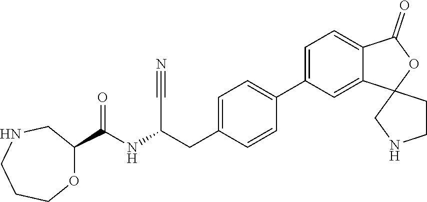 [SMILES: N#C[C@H](Cc1ccc(-c2ccc3c(c2)C2(CCNC2)OC3=O)cc1)NC(=O)[C@@H]1CNCCCO1] |
| 26 | 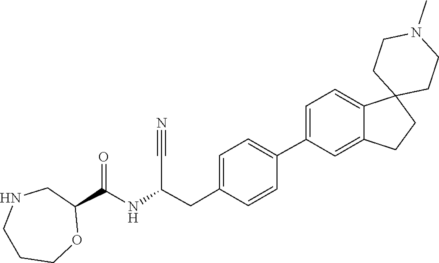 [SMILES: CN1CCC2(CCc3cc(-c4ccc(C[C@@H](C#N)NC(=O)[C@@H]5CNCCCO5)cc4)ccc32)CC1] |
| 27 | 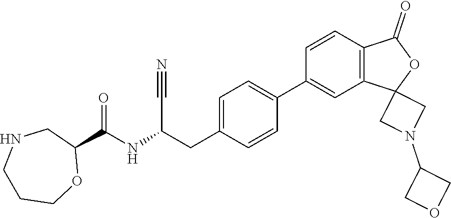 [SMILES: N#C[C@H](Cc1ccc(-c2ccc3c(c2)C2(CN(C4COC4)C2)OC3=O)cc1)NC(=O)[C@@H]1CNCCCO1] |
| 28 | 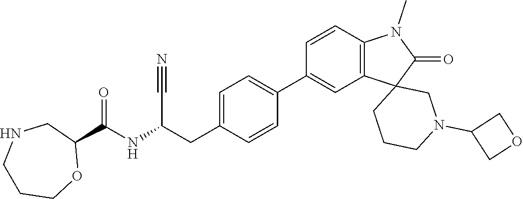 [SMILES: CN1C(=O)C2(CCCN(C3COC3)C2)c2cc(-c3ccc(C[C@@H](C#N)NC(=O)[C@@H]4CNCCCO4)cc3)ccc21] |
| 29 | 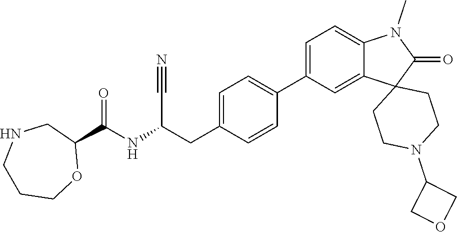 [SMILES: CN1C(=O)C2(CCN(C3COC3)CC2)c2cc(-c3ccc(C[C@@H](C#N)NC(=O)[C@@H]4CNCCCO4)cc3)ccc21] |
| 30 | 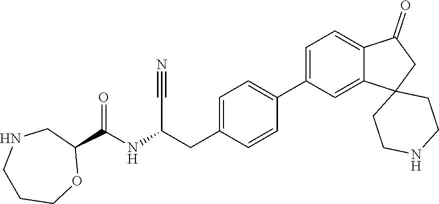 [SMILES: N#C[C@H](Cc1ccc(-c2ccc3c(c2)C2(CCNCC2)CC3=O)cc1)NC(=O)[C@@H]1CNCCCO1] |
| 31 | 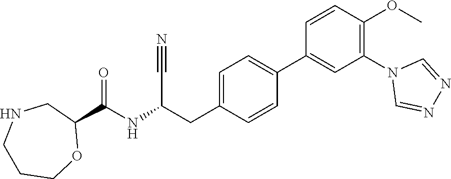 [SMILES: COc1ccc(-c2ccc(C[C@@H](C#N)NC(=O)[C@@H]3CNCCCO3)cc2)cc1-n1cnnc1] |
| 32 | 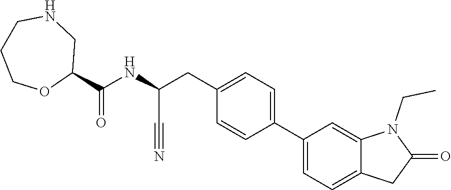 [SMILES: CCN1C(=O)Cc2ccc(-c3ccc(C[C@@H](C#N)NC(=O)[C@@H]4CNCCCO4)cc3)cc21] |
| 33 | 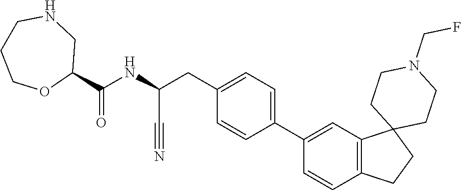 [SMILES: N#C[C@H](Cc1ccc(-c2ccc3c(c2)C2(CC3)CCN(CF)CC2)cc1)NC(=O)[C@@H]1CNCCCO1] |
| 34 | 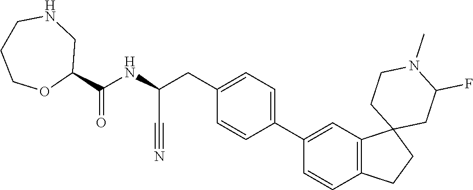 [SMILES: CN1CCC2(CCc3ccc(-c4ccc(C[C@@H](C#N)NC(=O)[C@@H]5CNCCCO5)cc4)cc32)CC1F] |
| 35 | 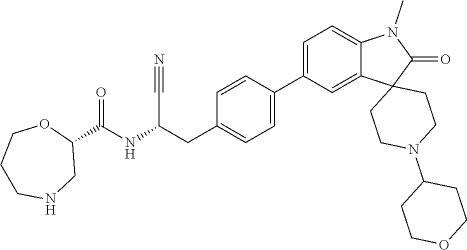 [SMILES: CN1C(=O)C2(CCN(C3CCOCC3)CC2)c2cc(-c3ccc(C[C@@H](C#N)NC(=O)[C@@H]4CNCCCO4)cc3)ccc21] |
| 36 | 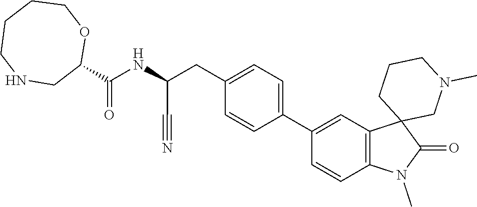 [SMILES: CN1CCCC2(C1)C(=O)N(C)c1ccc(-c3ccc(C[C@@H](C#N)NC(=O)[C@@H]4CNCCCCO4)cc3)cc12] |
| 37 | 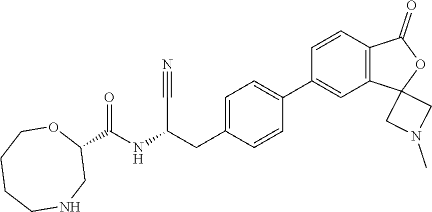 [SMILES: CN1CC2(C1)OC(=O)c1ccc(-c3ccc(C[C@@H](C#N)NC(=O)[C@@H]4CNCCCCO4)cc3)cc12] |
| 38 | 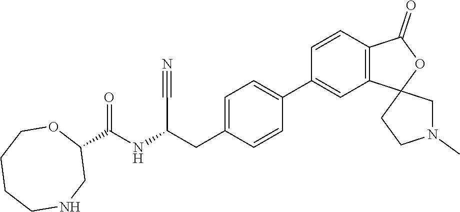 [SMILES: CN1CCC2(C1)OC(=O)c1ccc(-c3ccc(C[C@@H](C#N)NC(=O)[C@@H]4CNCCCCO4)cc3)cc12] |
| 39 | 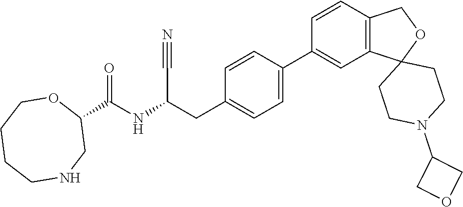 [SMILES: N#C[C@H](Cc1ccc(-c2ccc3c(c2)C2(CCN(C4COC4)CC2)OC3)cc1)NC(=O)[C@@H]1CNCCCCO1] |
| 40 | 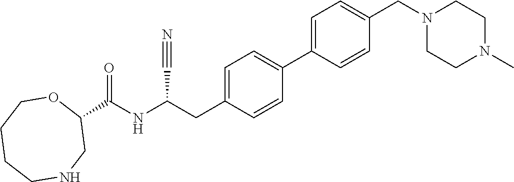 [SMILES: CN1CCN(Cc2ccc(-c3ccc(C[C@@H](C#N)NC(=O)[C@@H]4CNCCCCO4)cc3)cc2)CC1] |
| 41 | 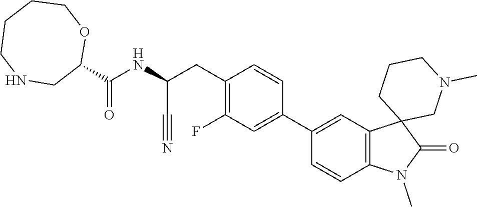 [SMILES: CN1CCCC2(C1)C(=O)N(C)c1ccc(-c3ccc(C[C@@H](C#N)NC(=O)[C@@H]4CNCCCCO4)c(F)c3)cc12] |
| 42 | 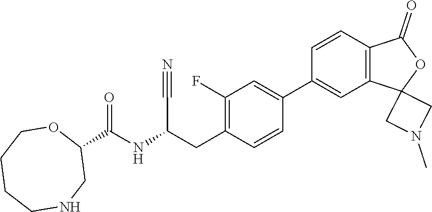 [SMILES: CN1CC2(C1)OC(=O)c1ccc(-c3ccc(C[C@@H](C#N)NC(=O)[C@@H]4CNCCCCO4)c(F)c3)cc12] |
| 43 | 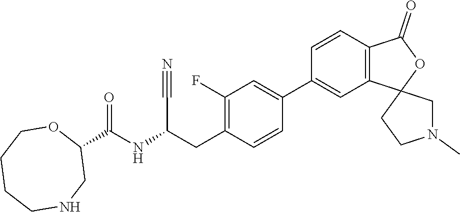 [SMILES: CN1CCC2(C1)OC(=O)c1ccc(-c3ccc(C[C@@H](C#N)NC(=O)[C@@H]4CNCCCCO4)c(F)c3)cc12] |
| 44 | 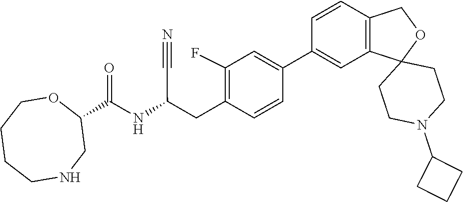 [SMILES: N#C[C@H](Cc1ccc(-c2ccc3c(c2)C2(CCN(C4CCC4)CC2)OC3)cc1F)NC(=O)[C@@H]1CNCCCCO1] |
| 45 | 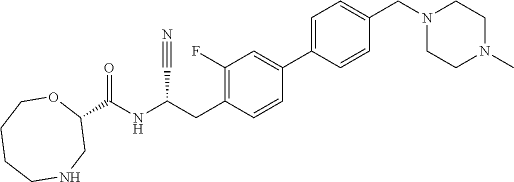 [SMILES: CN1CCN(Cc2ccc(-c3ccc(C[C@@H](C#N)NC(=O)[C@@H]4CNCCCCO4)c(F)c3)cc2)CC1] |
| 46 | 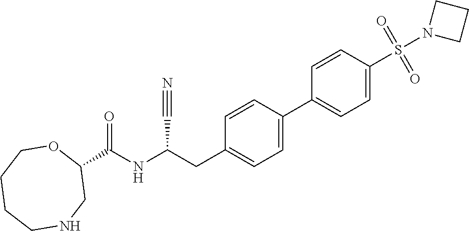 [SMILES: N#C[C@H](Cc1ccc(-c2ccc(S(=O)(=O)N3CCC3)cc2)cc1)NC(=O)[C@@H]1CNCCCCO1] |
| 47 | 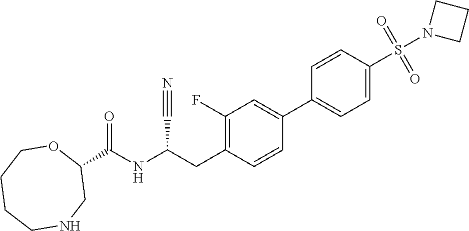 [SMILES: N#C[C@H](Cc1ccc(-c2ccc(S(=O)(=O)N3CCC3)cc2)cc1F)NC(=O)[C@@H]1CNCCCCO1] |
| 48 | 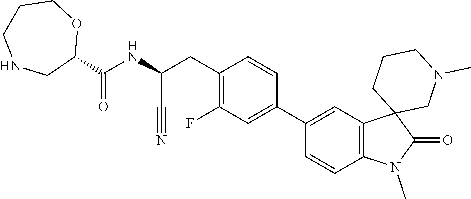 [SMILES: CN1CCCC2(C1)C(=O)N(C)c1ccc(-c3ccc(C[C@@H](C#N)NC(=O)[C@@H]4CNCCCO4)c(F)c3)cc12] |
| 49 | 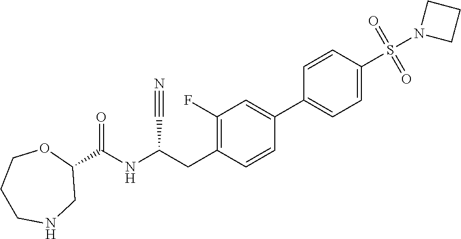 [SMILES: N#C[C@H](Cc1ccc(-c2ccc(S(=O)(=O)N3CCC3)cc2)cc1F)NC(=O)[C@@H]1CNCCCO1] |

In embodiments, provided herein is a compound in Table K, or a pharmaceutically acceptable salt thereof, racemic form thereof, or stereoisomer thereof.

In embodiments of the present disclosure, the compound is not a compound in Table K, or a pharmaceutically acceptable salt thereof, racemic form thereof, or stereoisomer thereof.

TABLE K
Compounds

| Compound No. | Structure |
|---|---|
| B5 | 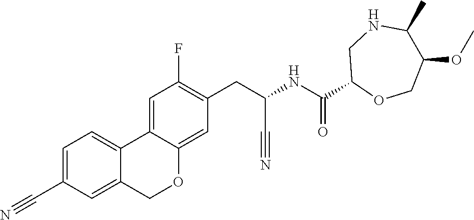 [SMILES: CO[C@H]1CO[C@H](C(=O)N[C@H](C#N)Cc2cc3c(cc2F)-c2ccc(C#N)cc2CO3)CN[C@H]1C] |
| B6 | 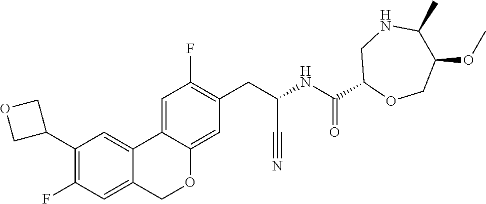 [SMILES: CO[C@H]1CO[C@H](C(=O)N[C@H](C#N)Cc2cc3c(cc2F)-c2cc(C4COC4)c(F)cc2CO3)CN[C@H]1C] |
| E1 | 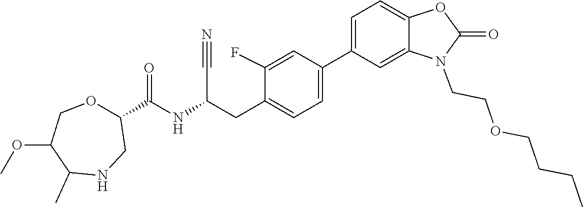 [SMILES: CCCCOCCn1c(=O)oc2ccc(-c3ccc(C[C@@H](C#N)NC(=O)[C@@H]4CNC(C)C(OC)CO4)c(F)c3)cc21] |
| E2 | 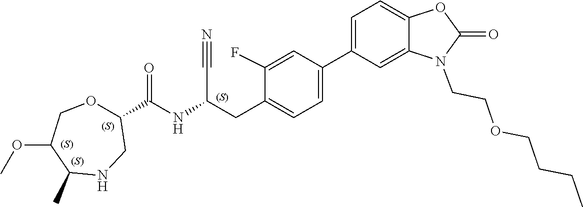 |
| E3 | 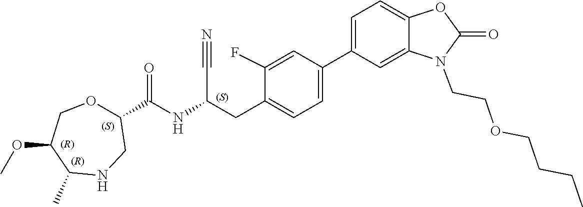 |
| E4 | 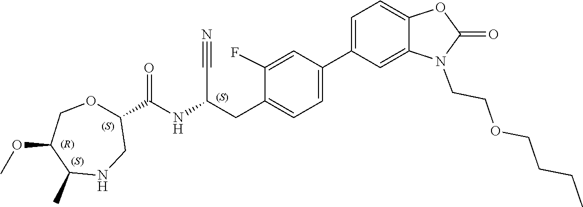 |
| E5 | 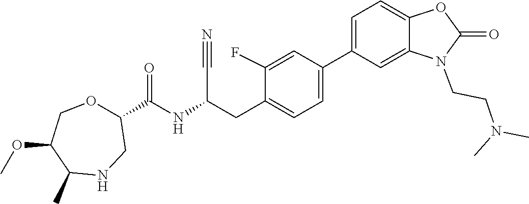 |
| E6 | 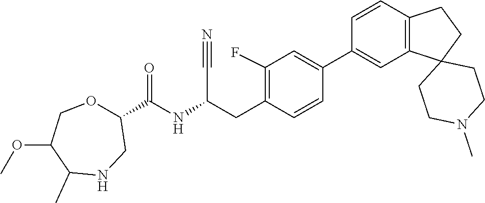 [SMILES: COC1CO[C@H](C(=O)N[C@H](C#N)Cc2ccc(-c3ccc4c(c3)C3(CC4)CCN(C)CC3)cc2F)CNC1C] |
| E7 | 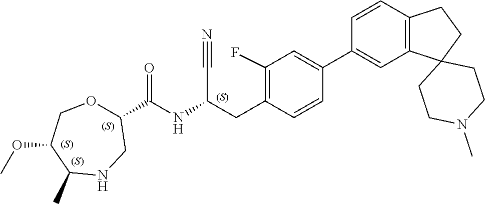 |
| E8 | 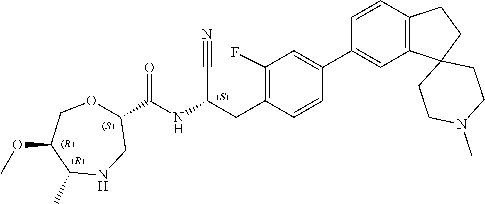 |
| E9 | 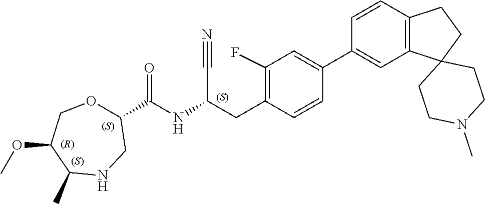 |
| E11 | 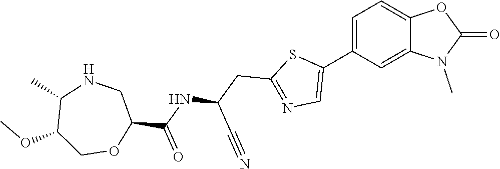 [SMILES: CO[C@H]1CO[C@H](C(=O)N[C@H](C#N)Cc2ncc(-c3ccc4oc(=O)n(C)c4c3)s2)CN[C@H]1C] |
| E13 | 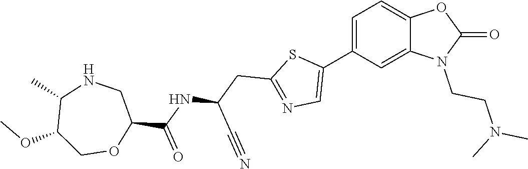 |
| E15 | 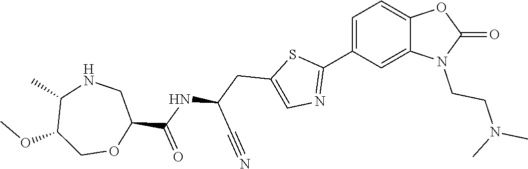 |
| E17 | 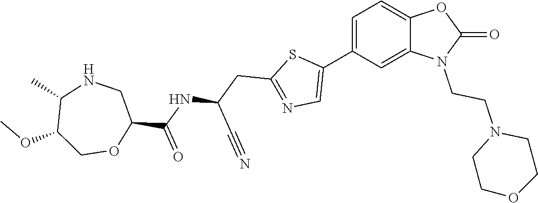 [SMILES: CO[C@H]1CO[C@H](C(=O)N[C@H](C#N)Cc2ncc(-c3ccc4oc(=O)n(CCN5CCOCC5)c4c3)s2)CN[C@H]1C] |

### Compositions

The compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or pharmaceutically acceptable salts thereof, or deuterated versions of the foregoing, may be used on their own but will generally be administered in the form of a pharmaceutical composition in which the compounds of formula (I), (I′), (I-A), (I-B), (I-C), (I-D), (I-E), (II), Tables A-K, or pharmaceutically acceptable salts thereof, or deuterated versions thereof (active ingredient) is in association with pharmaceutically acceptable adjuvant(s), diluents(s) or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

In embodiments, the present disclosure provides pharmaceutical composition(s) comprising a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt thereof, as hereinbefore defined in association with pharmaceutically acceptable adjuvant(s), diluent(s) or carrier(s).

The disclosure further provides a process for the preparation of a pharmaceutical composition of the disclosure which comprises mixing a compound of (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt thereof, as hereinbefore defined with a pharmaceutically acceptable adjuvant(s), diluents(s) or carrier(s).

The pharmaceutical compositions may be administered topically (e.g., to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g., by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatin or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatin, talcum and titanium dioxide.

For the preparation of soft gelatin capsules, the compound of the disclosure may be admixed with, for example, a vegetable oil or polyethylene glycol. Hard gelatin capsules may contain granules of the compound using pharmaceutical excipients like the abovementioned excipients for tablets. Additionally, liquid or semisolid formulations of the compound of the disclosure may be filled into hard gelatin capsules.

Liquid preparations for oral application may be in the form of syrups, solutions or suspensions. Solutions, for example may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

### Therapeutic Use

In embodiments, the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, and their pharmaceutically acceptable salts or deuterated form thereof, are DPP1 inhibitors, and thus may be used in any disease area where DPP1 plays a role. As such, in one aspect of the disclosure, a method of treatment is provided. The method of treatment, in one embodiment, comprises, administering to a subject in need thereof, a composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt thereof, a deuterated form thereof, a racemic form thereof, or a stereoisomer thereof. In embodiments, the composition is administered to the patient for an administration period.

In embodiments, a compound or composition of the present disclosure is administered to a patient in a method for treating a obstructive disease of the airway; chronic rhinosinusitis (CRS); hidradenitis suppurativa (HS); cancer (e.g., cancer metastasis); granulomatosis with polyangiitis (GPA); microscopic polyangiitis (MPA); giant cell arteritis; polyarteritis nodosa; anti-GBM disease (Goodpasture's); rheumatoid arthritis; lupus nephritis; systemic lupus erythematosus; systemic scleroderma; inflammatory bowel disease (IBD) (e.g., ulcerative colitis; Crohn's disease); diabetic nephropathy; diabetic neuropathy; diabetic retinopathy; diabetic ulcers; Duchenne muscular dystrophy; bronchiolitis obliterans; long covid)—prophylaxis of ILD; atopic dermatitis; pyoderma gangrenosum; sweet's syndrome; dermatomyositis/polymyositis; neutrophilic dermatoses; uveitis; Behcet's disease; thrombosis including deep vein thrombosis (DVT); bronchopulmonary dysplasia; amyotrophic lateral sclerosis; sickle cell anemia; psoriasis; ventilator-induced lung injury.

In embodiments, a compound or composition of the present disclosure is administered to a patient in a method for treating thrombosis. In embodiments, the thrombosis is deep vein thrombosis (DVT).

In embodiments, a compound or composition of the present disclosure is administered to a patient in a method for treating an obstructive disease of the airway. The obstructive disease of the airway, in one embodiment, is asthma (e.g., bronchial, allergic, intrinsic, extrinsic, exercise-induced, neutrophilic, drug-induced (including aspirin and NSAID-induced) asthma, dust-induced asthma, and both intermittent and persistent asthma and asthma of all severities) airway hyper-responsiveness, chronic obstructive pulmonary disease (COPD), bronchitis (e.g., infectious bronchitis, eosinophilic bronchitis), emphysema, cystic fibrosis (CF), bronchiectasis (e.g., non-CF bronchiectasis (NCFBE) and bronchiectasis associated with CF), cystic fibrosis; sarcoidosis; alpha-1 antitrypsin (A1AT) deficiency, farmer's lung and related diseases, hypersensitivity pneumonitis, interstitial lung disease, pulmonary fibrosis (also known as lung fibrosis) including idiopathic pulmonary fibrosis, cryptogenic fibrosing alveolitis, idiopathic interstitial pneumonias, fibrosis complicating anti-neoplastic therapy and chronic infection, including tuberculosis and aspergillosis and other fungal infections), complications of lung transplantation, vasculitic and thrombotic disorders of the lung vasculature, pulmonary hypertension (e.g., pulmonary arterial hypertension), antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus (e.g., respiratory syncytial virus, influenza, coronavirus (including SARS) and adenovirus), acute lung injury, acute respiratory distress syndrome (ARDS), as well as exacerbations of each of the foregoing respiratory tract disease states. In embodiments, asthma is neutrophilic asthma.

In embodiments, a compound or composition of the present disclosure is administered to a patient in a method for treating pulmonary hypertension. In some embodiment, pulmonary hypertension is pulmonary arterial hypertension. In some embodiments, pulmonary hypertension is pulmonary hypertension due to left heart disease. In some embodiments, pulmonary hypertension is pulmonary hypertension associated with chronic lung disease.

Cystic fibrosis (CF) is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. The inflammatory process is dominated by neutrophils that produce NE, as well as other destructive NSPs including CatG and PR3, that directly act upon extracellular matrix proteins and play a role in the host response to inflammation and infection (Dittrich et al., Eur Respir J. 2018; 51(3)). The methods provided herein employ reversible inhibitors of DPP1. Without wishing to be bound by theory, it is thought that the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof, administered via the methods provided herein have beneficial effects via inhibiting the activation of NSPs and decreasing inflammation, which in turn leads to a decrease in pulmonary exacerbations, a decrease in the rate of pulmonary exacerbations, and/or an improvement in lung function (e.g., forced expiratory volume in 1 second [FEV₁]) in CF patients.

In one embodiment, a method is provided for treating CF comprising administering to a CF patient in need of treatment, a composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof.

In one CF treatment method, a composition comprising an effective amount of a compound of (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof, is administered to a CF patient in need of treatment for an administration period. The method comprises improving the lung function of the patient during the administration period, as compared to the lung function of the patient prior to the administration period. The improvement in lung function in one embodiment, is measured by spirometry.

Improving the lung function of the patient, in one embodiment, comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75%)}), as compared to the respective value prior to the administration period. Increasing, in one embodiment, is by about 5%, by about 10%, by about 15%, by about 20%, by about 25%, by about 30%, by about 35%, by about 40%, by about 45% or by about 50% of the respective value. Increasing, in one embodiment, is by at least about 5%, by at least about 10%, by at least about 15%, by at least about 20%, by at least about 25%, by at least about 30%, by at least about 35%, by at least about 40%, by at least about 45% or by at least about 50%. In yet another embodiment, the increasing is by about 5% to about 50%, by about 5% to about 40%, by about 5% to about 30% or by about 5% to about 20%. In even another embodiment, increasing is by about 10% to about 50%, by about 15% to about 50%, by about 20% to about 50%, or by about 25% to about 50%.

In one embodiment of a method provided herein, a composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof, is administered to a bronchiectasis patient in need of treatment for an administration period. Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65(Suppl 1), incorporated by reference herein in its entirety for all purposes).

Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65(Suppl 1), incorporated by reference herein in its entirety for all purposes).

The methods provided herein employ reversible inhibitors of DPP1. Without wishing to be bound by theory, it is thought that the compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof, administered via the methods provided herein have beneficial effects via decreasing inflammation and mucus hypersecretion, which in some embodiments, leads to a decrease in pulmonary exacerbations, a decrease in the rate of pulmonary exacerbations, and/or an improvement in lung function (cough, sputum production, and forced expiratory volume in 1 second [FEV₁]) in bronchiectasis patients. Without wishing to be bound by theory, it is thought that the methods provided herein modify bronchiectasis progression by reducing the accelerated rate of lung function decline or lung tissue destruction.

In one embodiment, the bronchiectasis is non-CF bronchiectasis.

In one embodiment, the method for treating bronchiectasis comprises improving lung function of the patient during the administration period, as compared to the lung function of the patient prior to the administration period.

A pulmonary exacerbation, in one embodiment, is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis. In a further embodiment, the three or more symptoms result in a physician's decision to prescribe an antibiotic(s) to the patient exhibiting the symptoms.

In one embodiment of a method for treating bronchiectasis, the method comprises decreasing the rate of pulmonary exacerbation in the subject, compared to the rate of pulmonary exacerbation experienced by the subject prior to the administration period of the composition, or compared to a control subject with bronchiectasis that is not subject to the method of treatment. In a further embodiment, the bronchiectasis is non-CF bronchiectasis.

In another aspect, a method for treating chronic rhinosinusitis (CRS) in a subject in need thereof is provided. The method comprises in one embodiment, administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof.

The chronic rhinosinusitis is chronic rhinosinusitis without nasal polyps (CRSsNP), or chronic rhinosinusitis with nasal polyps (CRSwNP). In some embodiments, the chronic rhinosinusitis is chronic rhinosinusitis without nasal polyps (CRSsNP). In some embodiments, the chronic rhinosinusitis is chronic rhinosinusitis with nasal polyps (CRSwNP). In some embodiments, the chronic rhinosinusitis is refractory chronic rhinosinusitis. In some embodiments, the refractory chronic rhinosinusitis is refractory chronic rhinosinusitis without nasal polyps (CRSsNP). In some embodiments, the refractory chronic rhinosinusitis is refractory chronic rhinosinusitis with nasal polyps (CRSwNP).

In some embodiments, the subject exhibits one or more symptoms of CRS. In some embodiments, the one or more symptoms of CRS are: (a) nasal congestion; (b) nasal obstruction; (c) nasal discharge; (d) post-nasal drip; I facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; or (o) any combinations thereof. In some embodiments, obstruction of the middle meatus is mucosal obstruction, edematous obstruction, or a combination thereof.

In some embodiments, the administration of the pharmaceutical composition reduces, diminishes the severity of, delays the onset of, or eliminates one or more symptoms of CRS. In some embodiments, the one or more symptoms of CRS are: (a) nasal congestion; (b) nasal obstruction; (c) nasal discharge; (d) post-nasal drip; I facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; (o) or any combinations thereof. In some embodiments, the administration of the pharmaceutical composition enhances sinus drainage.

In some embodiments, the methods comprise reducing a composite severity score of one or more symptoms of CRS. As used herein, the “composite severity score” is a quantitative measure of all the symptoms of CRS exhibited by the subject. In some embodiments, the composite severity score is a sum total of all the daily symptoms exhibited by the subject. In some embodiments, the composite severity score is reduced during or subsequent to the administration period, as compared to the composite severity score measured prior to the administration period. In some embodiments, the one or more symptoms of CRS exhibited by the subject may be any symptoms described herein or known in the art to be associated with CRS. In some embodiments, the one or more symptoms of CRS are: nasal congestion, reduced smell, rhinorrhea, or any combination thereof. In some embodiments, the rhinorrhea is anterior rhinorrhea. In some embodiments, the rhinorrhea is posterior rhinorrhea.

In some embodiments, the methods comprise decreasing the Sino-Nasal Outcome Test-22 (SNOT-22) score of the subject during the administration period or subsequent to the administration period, compared to the SNOT-22 score of the subject prior to the administration period. As used herein, “SNOT-22” is a patient-reported measure of outcome developed for use in CRS with or without nasal polyps and contains 22 individual questions. The questions cover a broad range of health and health-related quality of life problems including physical problems, functional limitations and emotional consequences. The theoretical range of the SNOT-22 score is 0-110, with lower scores implying a better health-related quality of life. Further details of SNOT-22 are provided in Hopkins, et al., Clin. Otolaryngol. 2009, 34, 447-454, and Kennedy, et al., Ann Allergy Asthma Immunol. 2013 October; 111(4): 246-251, the contents of which are incorporated herein by reference in its entirety.

Hidradenitis suppurativa (HS) is a chronic relapsing inflammatory disorder. The symptoms include skin lesions that are often associated hair follicles, and may be painful, inflamed and/or swollen. In some cases, when the skin lesions heal, they can recur, and may lead to tunnels under the skin and progressive scarring. Since HS is a chronic condition, it can persist for many years and also, worsen over time, with serious effects on quality of life, psychological and emotional well-being. In fact, HS patients have increased rates of anxiety and depression with a risk of suicide two and a half times that of the general population.

HS patients are categorized according to disease severity, termed Hurley staging, as mild (Stage I), moderate (Stage II), or severe (Stage III). Although more than 200,000 cases of HS are diagnosed in the U.S. per year, this disease can be difficult to diagnose and requires specialized care. HS may be mistaken for an infection, an ingrown hair or other conditions. Moreover, current treatment options are limited and lack efficacy.

In one aspect, a method of treating HS in a subject in need thereof is provided. The method comprises in one embodiment, administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof. In a further embodiment, the method of treating HS comprises reducing neutrophilic inflammation in the subject.

The HS in one embodiment, is Hurley Stage I HS, Hurley Stage II HS or Hurley Stage III HS. In some embodiments, the HS is Hurley Stage I HS. In some embodiments, the HS is Hurley Stage II HS. In some embodiments, the HS is Hurley Stage III HS.

The disclosure provides methods of treating cancer in a subject in need thereof, comprising, administering to the subject, a pharmaceutical composition comprising an effective amount of any one of the compounds disclosed herein. The disclosure provides methods of treating cancer-induced pain in a subject having cancer, comprising, administering to the subject for an administration period, a pharmaceutical composition comprising an effective amount of any one of the compounds disclosed herein. In some embodiments, the cancer-induced pain is cancer-induced bone pain. The disclosure also provides methods of treating cancer-induced bone pain in a subject having cancer, comprising, administering to the subject for an administration period, a pharmaceutical composition comprising an effective amount of any one of the compounds disclosed herein.

In some embodiments, the cancer comprises a primary solid tumor. In some embodiments, the cancer is bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, or bone cancer.

In some embodiments, the cancer is bladder cancer. In some embodiments, the cancer is lung cancer. In some embodiments, the cancer is brain cancer. In some embodiments, the cancer is ovarian cancer. In some embodiments, the cancer is pancreatic cancer. In some embodiments, the cancer is colorectal cancer. In some embodiments, the cancer is prostate cancer. In some embodiments, the cancer is liver cancer. In some embodiments, the cancer is hepatocellular carcinoma. In some embodiments, the cancer is kidney cancer. In some embodiments, the cancer is stomach cancer. In some embodiments, the cancer is skin cancer. In some embodiments, the cancer is fibroid cancer. In some embodiments, the cancer is lymphoma. In some embodiments, the cancer Is virus-induced cancer. In some embodiments, the cancer is oropharyngeal cancer. In some embodiments, the cancer is testicular cancer. In some embodiments, the cancer is thymus cancer. In some embodiments, the cancer is thyroid cancer. In some embodiments, the cancer is melanoma. In some embodiments, the cancer is bone cancer. In some embodiments, the fibroid cancer is leiomyosarcoma.

In some embodiments, the breast cancer comprises ductal carcinoma, lobular carcinoma, medullary carcinoma, colloid carcinoma, tubular carcinoma, or inflammatory breast cancer. In some embodiments, the breast cancer comprises ductal carcinoma. In some embodiments, the breast cancer comprises lobular carcinoma. In some embodiments, the breast cancer comprises medullary carcinoma. In some embodiments, the breast cancer comprises colloid carcinoma. In some embodiments, the breast cancer comprises tubular carcinoma. In some embodiments, the breast cancer comprises inflammatory breast cancer.

In some embodiments, the breast cancer is triple-negative breast cancer. In some embodiments, the breast cancer does not respond to hormonal therapy or therapeutics that target the HER2 protein receptors.

In some embodiments, the lymphoma is Hodgkin's lymphoma, non-Hodgkin's lymphoma, diffuse large B-cell lymphoma, B-cell immunoblastic lymphoma, Natural Killer cell lymphoma, T-cell lymphoma, Burkitt lymphoma or Kaposi's Sarcoma. In some embodiments, the lymphoma is Hodgkin's lymphoma. In some embodiments, the lymphoma is non-Hodgkin's lymphoma. In some embodiments, the lymphoma is diffuse large B-cell lymphoma. In some embodiments, the lymphoma is B-cell immunoblastic lymphoma. In some embodiments, the lymphoma is Natural Killer cell lymphoma. In some embodiments, the lymphoma is T-cell lymphoma. In some embodiments, the lymphoma is Burkitt lymphoma. In some embodiments, the lymphoma is Kaposi's Sarcoma.

In some embodiments, the brain cancer is astrocytoma, anaplastic astrocytoma, glioblastoma multiforme, oligodendroglioma, ependymoma, meningioma, schwannoma, or medulloblastoma. In some embodiments, the brain cancer is astrocytoma. In some embodiments, the brain cancer is anaplastic astrocytoma. In some embodiments, the brain cancer is glioblastoma multiforme. In some embodiments, the brain cancer is oligodendroglioma. In some embodiments, the brain cancer is ependymoma. In some embodiments, the brain cancer is meningioma. In some embodiments, the brain cancer is schwannoma. In some embodiments, the brain cancer is medulloblastoma.

In some embodiments, the cancer is liquid tumor. In some embodiments, the liquid tumor is acute myeloid leukemia (AML), acute lymphoblastic leukemia, acute lymphocytic leukemia, acute promyelocytic leukemia, chronic myeloid leukemia, hairy cell leukemia, a myeloproliferative disorder, Natural Killer cell leukemia, blastic plasmacytoid dendritic cell neoplasm, chronic myelogenous leukemia (CML), mastocytosis, chronic lymphocytic leukemia (CLL), multiple myeloma (MM), or myelodysplastic syndrome (MDS). In some embodiments, the liquid tumor is acute myeloid leukemia (AML). In some embodiments, the liquid tumor is acute lymphoblastic leukemia. In some embodiments, the liquid tumor is acute lymphocytic leukemia. In some embodiments, the liquid tumor is acute promyelocytic leukemia. In some embodiments, the liquid tumor is chronic myeloid leukemia. In some embodiments, the liquid tumor is hairy cell leukemia. In some embodiments, the liquid tumor is a myeloproliferative disorder. In some embodiments, the liquid tumor is Natural Killer cell leukemia. In some embodiments, the liquid tumor is blastic plasmacytoid dendritic cell neoplasm. In some embodiments, the liquid tumor is chronic myelogenous leukemia (CML). In some embodiments, the liquid tumor is mastocytosis. In some embodiments, the liquid tumor is chronic lymphocytic leukemia (CLL). In some embodiments, the liquid tumor is multiple myeloma (MM). In some embodiments, the liquid tumor is myelodysplastic syndrome (MDS).

In some embodiments, the cancer is a pediatric cancer. In some embodiments, the pediatric cancer is neuroblastoma, Wilms tumor, rhabdomyosarcoma, retinoblastoma, osteosarcoma or Ewing sarcoma. In some embodiments, the pediatric cancer is neuroblastoma. In some embodiments, the pediatric cancer is Wilms tumor. In some embodiments, the pediatric cancer is rhabdomyosarcoma. In some embodiments, the pediatric cancer is retinoblastoma. In some embodiments, the pediatric cancer is osteosarcoma. In some embodiments, the pediatric cancer is Ewing sarcoma.

In some embodiments, the cancer is metastatic cancer. In some embodiments, the subject is at a risk for developing metastatic cancer. In some embodiments, the metastatic cancer comprises metastasis of breast cancer to the brain, bone, pancreas, lymph nodes, and/or liver. In some embodiments, the metastatic cancer comprises metastasis of bone cancer to the lung. In some embodiments, the metastatic cancer comprises metastasis of colorectal cancer to the peritoneum, the pancreas, the stomach, the lung, the liver, the kidney, and/or the spleen. In some embodiments, the metastatic cancer comprises metastasis of stomach cancer to the mesentery, the spleen, the pancreas, the lung, the liver, the adrenal gland, and/or the ovary. In some embodiments, the metastatic cancer comprises metastasis of leukemia to the lymph nodes, the lung, the liver, the hind limb, the brain, the kidney, and/or the spleen. In some embodiments, the metastatic cancer comprises metastasis of liver cancer to the intestine, the spleen, the pancreas, the stomach, the lung, and/or the kidney. In some embodiments, the metastatic cancer comprises metastasis of lymphoma to the kidney, the ovary, the liver, the bladder, and/or the spleen.

In some embodiments, the metastatic cancer comprises metastasis of hematopoietic cancer to the intestine, the lung, the liver, the spleen, the kidney, and/or the stomach. In some embodiments, the metastatic cancer comprises metastasis of melanoma to lymph nodes and/or the lung. In some embodiments, the metastatic cancer comprises metastasis of pancreatic cancer to the mesentery, the ovary, the kidney, the spleen, the lymph nodes, the stomach, and/or the liver. In some embodiments, the metastatic cancer comprises metastasis of prostate cancer to the lung, the pancreas, the kidney, the spleen, the intestine, the liver, the bone, and/or the lymph nodes. In some embodiments, the metastatic cancer comprises metastasis of ovarian cancer to the diaphragm, the liver, the intestine, the stomach, the lung, the pancreas, the spleen, the kidney, the lymph nodes, and/or the uterus. In some embodiments, the metastatic cancer comprises metastasis of myeloma to the bone.

In some embodiments, the metastatic cancer comprises metastasis of lung cancer to the bone, the brain, the lymph nodes, the liver, the ovary, and/or the intestine. In some embodiments, the metastatic cancer comprises metastasis of kidney cancer to the liver, the lung, the pancreas, the stomach, the brain, and/or the spleen. In some embodiments, the metastatic cancer comprises metastasis of bladder cancer to the bone, the liver and/or the lung. In some embodiments, the metastatic cancer comprises metastasis of thyroid cancer to the bone, the liver and/or the lung.

In some embodiments, the methods disclosed herein comprise treating cancer-induced bone pain (CIBP) in a subject having metastasis of a cancer to the bone. In some embodiments, the subject has metastasis of prostate cancer, breast cancer, lung cancer, or myeloma to the bone. In some embodiments, the subject is identified as having metastasis to the bone by the use of any one of the following methods: plain film radiography, computed tomography, technetium 99m bone scan, magnetic resonance imaging, fluorodeoxyglucose positron emission tomography, fluorine positron emission tomography, and/or choline positron emission tomography, but is not yet feeling cancer-induced bone pain. In some embodiments, the subject is suffering from cancer-induced bone pain, which is indicative of metastasis of a previously treated or untreated primary tumor to the bone. In some embodiments, the cancer has metastasized to vertebrae, pelvis, long bones, or ribs.

In some embodiments, administration of the composition diminishes the severity of, delays the onset of, or eliminates a symptom of cancer. In some embodiments, the symptom of cancer is cancer-induced bone pain (CIBP). In some embodiments, the CIBP is neuropathic pain. In some embodiments, the CIBP is inflammatory pain. In some embodiments, the CIBP is spontaneous pain. In some embodiments, the symptom of cancer is nociceptive hypersensitivity. In some embodiments, the symptom of cancer is allodynia. In some embodiments, the allodynia is tactile allodynia. In some embodiments, the tactile allodynia is static mechanical allodynia. In some embodiments, the tactile allodynia is dynamic mechanical allodynia. In some embodiments, the subject has bone cancer or metastasis to the bone.

In yet another embodiment of the present disclosure, a method for treating lupus nephritis (LN) in a subject in need thereof is provided. The method comprises administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof.

In embodiments of the present disclosure, a method for treating arthritis in a subject in need thereof is provided. The method comprises administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof. In embodiments, arthritis is osteoarthritis. In embodiments, arthritis is rheumatoid arthritis.

Osteoarthritis (OA) is typically not autoimmune in origin and is typically a gradual, degenerative joint disease due to age-related chronic use or injury of the joints leading to cartilage breakdown, bone changes and local non-resolving synovial inflammation. In embodiments, the present disclosure provides a method for treating osteoarthritis (OA) in a patient in need thereof, comprising administering to the patient an effective amount of a compound disclosed herein (e.g., a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof). In embodiments, the treating of osteoarthritis (OA) comprises improving weight loss and/or inflamed paw volume of the patient during the administration period, as compared to the weight loss and/or inflamed paw volume of the patient prior reducing weight loss and/or inflamed paw volume of the patient during the administration period, as compared to the weight loss and/or inflamed paw volume of the patient prior to the administration period.

Rheumatoid arthritis (RA) is characterized by inflammation and thickening of the joint capsule, together with an effect on the underlying bone and cartilage. Currently, the cause of RA is unknown and no satisfactory cure for RA is available. While a number of therapeutic agents have been developed and utilized to alleviate pain and inflammation associated with the disease, such as disease-modifying antirheumatic drugs (DMARDs) and non-steroidal anti-inflammatory agents (NSAIDs), they often produce intolerable side effects. To addresses this and other needs, the present disclosure, in one embodiment, provides a method for treating RA using reversible inhibitors of DPP1 of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof. In one embodiment, a method of for treating RA in a subject in need thereof is provided, and comprises administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof. In a further embodiment, the method comprises reducing neutrophilic inflammation in the subject.

Inflammatory bowel disease (IBD) is a group of inflammatory conditions that affect the colon and small intestine. The most common IBDs are Crohn's disease and ulcerative colitis. The present disclosure, in one embodiment, addresses the need for novel IBD therapies. Specifically, in one embodiment, a method for treating an inflammatory bowel disease (IBD) in a subject in need thereof is provided. The method comprises administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof.

In a further embodiment, the IBD is Crohn's disease or ulcerative colitis. In even a further embodiment, the method comprises reducing neutrophilic inflammation in the subject.

In embodiments, a compound or composition of the present disclosure is administered to a patient in a method for treating heart failure. In some embodiment, heart failure is heart failure with reduced ejection fraction. In some embodiments, heart failure is heart failure with preserved ejection fraction.

In yet another embodiment of the disclosure, a method for treating ischemia/reperfusion (IR) injury is provide, comprising administering to a patient in need of treatment, a compound or composition of the present disclosure to the patient in need of treatment. The IR injury, in one embodiment, is due to Heart transplantation (HTX). As such, in one embodiment, the patient is a heart transplant recipient. In a further embodiment, the patient is administered a compound or composition of the present disclosure during heart transplantation or subsequent to heart transplantation. In one embodiment of this method, the patient is administered one of the compounds set forth in Tables A-C. In yet even a further embodiment, the compound is present in an oral composition and is administered once daily to the patient in need of treatment.

Treating the IR injury in one embodiment, comprises improving left-ventricular (LV) graft function. Graft function can be measured, in one embodiment, by measuring LV systolic function, e.g., by measuring left-ventricular systolic pressure (LVSP), developed pressure, maximal slope of systolic pressure increment (dP/dtₘₐₓ), and/or rate pressure product (mmHg*bpm).

In one embodiment, treating IR injury comprises increasing the patient's LVSP (mmHg) during or subsequent to the administration period, as compared to the patient's LVSP (mmHg) prior to the administration period. In one embodiment, treating IR injury comprises increasing the patient's developed pressure (mmHg) during or subsequent to the administration period, as compared to the patient's developed pressure (mmHg) prior to the administration period. In yet another embodiment, treating IR injury in a patient in need of treatment comprises increasing the maximal slope of systolic pressure increment (dP/dtₘₐₓ) for the patient during or subsequent to the administration period, as compared to the maximal slope of systolic pressure increment (dP/dtₘₐₓ) for the patient prior to the administration period. In even yet another embodiment, treating IR injury in a patient in need of treatment comprises increasing the patient's rate pressure product during or subsequent to the administration period, as compared to the patient's rate pressure product prior to the administration period.

In embodiments, a compound or composition of the present disclosure is administered to a patient in a method for treating liver injury. The method comprises administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof. In embodiments, the liver injury is acute liver injury. In embodiments, the liver injury is drug-induced acute liver injury. In one embodiment, the liver injury is acetaminophen (APAP)-induced acute liver injury. In one embodiment, the liver injury is caused by acetaminophen overdose. In embodiment, the liver injury is caused by nonsteroidal anti-inflammatory drugs (NSAIDs), such as ibuprofen, diclofenac, and naproxen. In one embodiment, the treatment of ALI is a prophylactic treatment

In embodiments, a compound or composition of the present disclosure is administered to a patient in a method for treating sepsis. The method comprises administering to the subject for an administration period, a pharmaceutical composition comprising an effective amount of a compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof. In one embodiment, sepsis is a consequence of the patient's response to overwhelming bacterial infection. In one embodiment, the treatment of sepsis prevents organ dysfunction and death of the patient.

The length of the administration period in any given case may depend on the nature and severity of the condition being treated and/or prevented and be determined by the physician. In one embodiment, the administration period starts at about the time of condition/disease diagnosis and continues for the lifetime of the patient.

In some embodiments, the administration period is about 30 days, about 35 days, about 40 days, about 45 days, about 50 days, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 30 months, about 36 months, about 4 years, about 5 years, about 10 years, about 15 years or about 20 years. In some embodiments, the compounds or compositions disclosed herein may be administered for a period of about 24 weeks. In some embodiments, the compounds or compositions disclosed herein may be administered for a period of about 52 weeks. In yet another embodiment, the administration period is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 30 months, at least about 36 months, at least about 4 years, at least about 5 years, at least about 10 years, at least about 15 years or at least about 20 years.

In some embodiments, the administration period for the methods provided herein is at least about 30 days, at least about 35 days, at least about 40 days, at least about 45 days, at least about 50 days, at least about 2 months, at least about 3 months, at least about 4 months or at least about 6 months, at least about 7 months, at least about 8 months, at least about 9 months, at least about 10 months, at least about 11 months, at least about 1 year, at least about 2 years, at least about 3 years, at least about 4 years, at least about 5 years. The administration period for the methods provided herein, in another embodiment, is from about 30 days to about 180 days. In another embodiment, the administration period is from about 30 days to about 36 months, or from about 30 days to about 30 months, or from about 30 days to about 24 months, or from about 30 days to about 18 months, or from about 30 days to about 12 months, or from about 30 days to about 6 months, or from about 6 months to about 30 months, or from about 6 months to about 24 months, or from about 6 months to about 18 months, or from about 12 months to about 36 months, or from about 12 months to about 24 months.

In one embodiment, the administration period is from about 1 year to about 30 years. For example, the administration period, in one embodiment, is from about 1 year to about 25 years, 1 year to about 20 years, from about 1 year to about 15 years, from about 1 year to about 10 years, from about 1 year to about 5 years, from about 1 year to about 3 years, from about 1 year to about 2 years, from about 2 years to about 15 years, from about 2 year to about 10 years, from about 2 years to about 8 years, from about 2 year to about 5 years, from about 2 years to about 4 years, or from about 2 years to about 3 years.

In one embodiment of the method, the subject is administered the composition once daily during the administration period. In another embodiment, the patient is administered the composition twice daily, or every other day, or once a week during the administration period. In another embodiment, administration is every other day, every third day, 3 times per week or 4 times per week during the administration period.

In embodiments, an effective amount of the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof or the composition comprising an effective amount of the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated is administered orally

In one embodiment, the oral dosage form is administered once daily during the administration period. In a further embodiment, the oral dosage form is administered at approximately the same time every day, e.g., prior to breakfast. In another embodiment, the composition comprising an effective amount of the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof is administered once a day or twice a day during the administration period. In yet another embodiment, the composition comprising an effective amount of the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof is administered once per week, every other day, every third day, twice per week, three times per week, four times per week, or five times per week during the administration period.

Administration, in one embodiment, is via the oral route. In a further embodiment, the composition is administered once daily.

The dosage administered will vary with the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof employed, the mode of administration, and the treatment outcome desired. For example, in one embodiment, the daily dosage of the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof if inhaled, may be in the range from 0.05 micrograms per kilogram body weight (g/kg) to 100 micrograms per kilogram body weight (g/kg). Alternatively, in one embodiment, if the compound of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof is administered orally, then the daily dosage of the compound of the disclosure may be in the range from 0.01 micrograms per kilogram body weight (g/kg) to 100 milligrams per kilogram body weight (mg/kg).

The compounds of formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof may be used on their own but will generally be administered in the form of a pharmaceutical composition in which the formula (I), (I′), (I-A), (I-B), (I-C), (I-D), (I-E), (II), (II-A), (II-B) (III), (III′), (III″), (III-A), (IV), (V), (V-A), (V-B), (VI), (VII), (VIII), or (IX), or Tables A-K, or a pharmaceutically acceptable salt, a stereoisomer, or a deuterated form thereof is in association with pharmaceutically acceptable adjuvant(s), diluents(s) or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002.

### EXAMPLES

The present disclosure is further illustrated by reference to the following Examples. However, it should be noted that these Examples, like the embodiments described above, are illustrative and are not to be construed as restricting the scope of the disclosure in any way.

In embodiments, compounds of the present disclosure can be synthesized using the following methods. General reaction conditions are given, and reaction products can be purified by generally known methods including silica gel chromatography using various organic solvents such as hexane, dichloromethane, ethyl acetate, methanol and the like or preparative reverse phase high pressure liquid chromatography.

### Example 1: Synthesis of (S)—N—((S)-1-cyano-2-(4-(3-fluoro-1-methyl-1H-indazol-6-yl)phenyl)ethyl)-1,4-oxazepane-2-carboxamide (Compound A1)

### Step 1. Synthesis of 6-bromo-3-fluoro-1-methylindazole

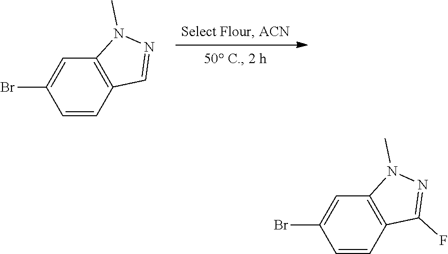

Into a 250 mL round bottom flask were added 6-bromo-1-methylindazole (6.0 g, 28.42 mmol, 1.0 equiv), Selectfluor (13.1 g, 36.95 mmol, 1.3 equiv) and ACN (60 mL) at room temperature. The resulting mixture was stirred for 2 h at 90° C. The reaction was cooled to room temperature, concentrated under reduced pressure to remove the solvent. The residue was purified by reversed-phase flash chromatography with the following conditions: column, C18-120 g, mobile phase, MeCN in Water (0.1% NH₃·H₂O), 10% to 80% gradient in 10 min; detector, UV 254 nm. This resulted in 6-bromo-3-fluoro-1-methylindazole (1.0 g, 15.3%) as white solid. LCMS (ES, m/z): [M+H]⁺: 229. ¹H NMR (400 MHz, DMSO-d₆) δ 8.03 (s, 1H), 7.68 (d, J=8.6 Hz, 1H), 7.32 (dd, J=8.7, 1.6 Hz, 1H), 3.92 (s, 3H).

### Step 2. Synthesis of tert-butyl (2S)-2-{[(1S)-1-cyano-2-[4-(3-fluoro-1-methylindazol-6-yl)phenyl]ethyl]carbamoyl}-1,4-oxazepane-4-carboxylate

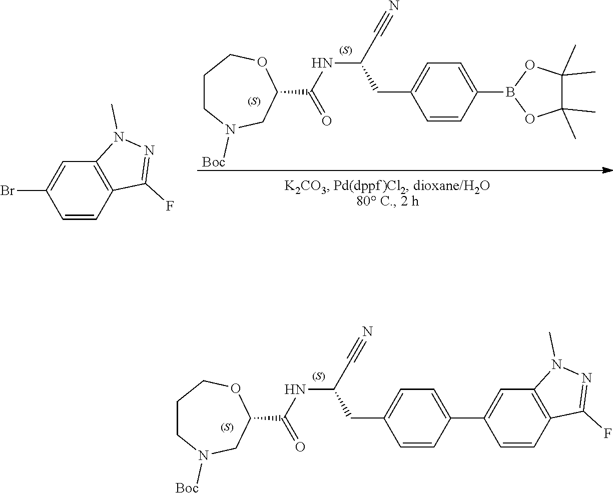

To a solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1.0 equiv), 6-bromo-3-fluoro-1-methylindazole (0.5 g, 2.00 mmol, 1.0 equiv) in 1,4-dioxane (15 ml) and H₂O (1.5 mL) were added Pd(dppf)Cl₂ (0.2 g, 0.20 mmol, 0.1 equiv) and K₂CO₃ (0.6 g, 4.00 mmol, 2.0 equiv) in sequence. The mixture was heated to 80° C. and stirred for 2 h under nitrogen gas atmosphere. The reaction was cooled to room temperature, concentrated under reduced pressure, the residue was purified by silica gel column chromatography, eluted with PE/THF (1:1) to afford tert-butyl (2S)-2-{[(1 S)-1-cyano-2-[4-(3-fluoro-1-methylindazol-6-yl)phenyl]ethyl]carbamoyl}-1,4-oxazepane-4-carboxylate (70 mg, 55.8%) as yellow oil. LCMS (ES, m/z): [M+H]⁺: 522.

### Step 3. Synthesis of (S)—N—((S)-1-cyano-2-(4-(3-fluoro-1-methyl-1H-indazol-6-yl)phenyl)ethyl)-1,4-oxazepane-2-carboxamide

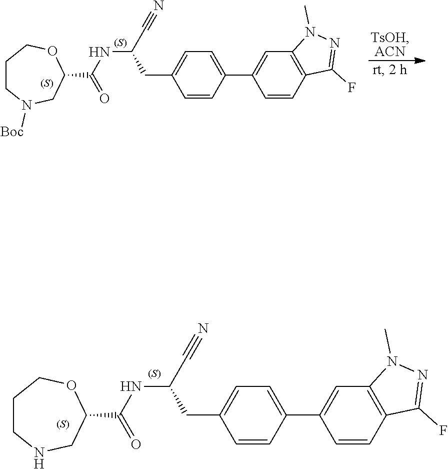

Into a 25 mL round-bottom flask were added tert-butyl (2S)-2-{[(1S)-1-cyano-2-[4-(3-fluoro-1-methylindazol-6-yl)phenyl]ethyl]carbamoyl}-1,4-oxazepane-4-carboxylate (600 mg, 1.15 mmol, 1.0 equiv) in ACN (18 mL) and TsOH (594 mg, 3.45 mmol, 3.0 equiv) at room temperature. The resulting mixture was stirred for 2 h at room temperature. The reaction solution was purified by reversed-phase flash chromatography with the following conditions: column, C18-120 g, mobile phase, MeCN in Water (0.1% NH₃·H₂O), 10% to 80% gradient in 10 min; detector, UV 254 nm. This resulted in Compound A1 (310.4 mg, 64%) as white solid. LCMS (ES, m/z): [M+H]⁺: 422.2. ¹H NMR (400 MHz, DMSO-d₆) δ 8.65 (d, J=8.5 Hz, 1H), 7.92 (s, 1H), 7.80-7.73 (m, 3H), 7.50 (dd, J=8.6, 1.4 Hz, 1H), 7.43 (d, J=8.0 Hz, 2H), 5.05 (q, J=8.2 Hz, 1H), 4.03 (dd, J=8.0, 3.7 Hz, 1H), 3.98 (s, 3H), 3.91-3.81 (m, 1H), 3.76-3.70 (m, 1H), 3.27-3.21 (m, 2H), 3.06 (dd, J=14.2, 3.6 Hz, 1H), 2.83-2.77 (m, 1H), 2.68-2.53 (m, 2H), 2.21 (br, 1H), 1.80-1.67 (m, 2H).

### Example 2: Synthesis of (2S)—N-[(1S)-1-cyano-2-[4-(1-oxo-3,4-dihydro-2H-isoquinolin-6-yl)phenyl]ethyl]-1,4-oxazepane-2-carboxamide (Compound A2)

### Step 1. Synthesis of tert-butyl (2S)-2-{[(1S)-1-cyano-2-[4-(1-oxo-3,4-dihydro-2H-isoquinolin-6-yl)phenyl]ethyl]carbamoyl}-1,4-oxazepane-4-carboxylate

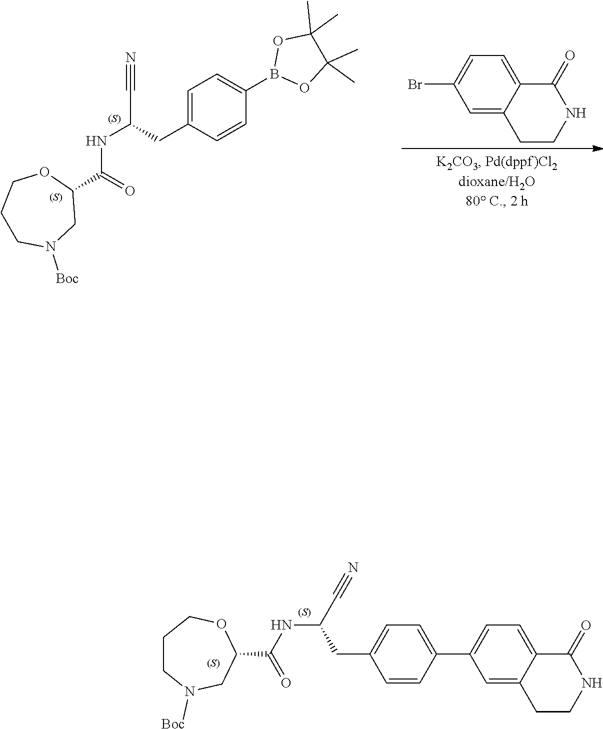

To a solution of 6-bromo-3,4-dihydro-2H-isoquinolin-1-one (54 mg, 0.24 mmol, 1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1 equiv) in dioxane (5 mL) and H₂O (0.5 mL) were added K₂CO₃ (66 mg, 0.48 mmol, 2 equiv) and Pd(dppf)Cl₂CH₂Cl₂ (20 mg, 0.024 mmol, 0.1 equiv). After stirring for 2 h at 80° C. under a nitrogen atmosphere, the resulting mixture was concentrated under reduced pressure. The residue was purified by silica gel column chromatography, eluted with PE/THF (2:1) to afford tert-butyl (2S)-2-{[(1S)-1-cyano-2-[4-(1-oxo-3,4-dihydro-2H-isoquinolin-6-yl)phenyl]ethyl]carbamoyl}-1,4-oxazepane-4-carboxylate (80 mg, 64.20%) as a light yellow oil. LCMS (ES, m/z): [M+H]⁺: 519.

### Step 2. Synthesis of (2S)—N-[(1S)-1-cyano-2-[4-(1-oxo-3,4-dihydro-2H-isoquinolin-6-yl)phenyl]ethyl]-1,4-oxazepane-2-carboxamide

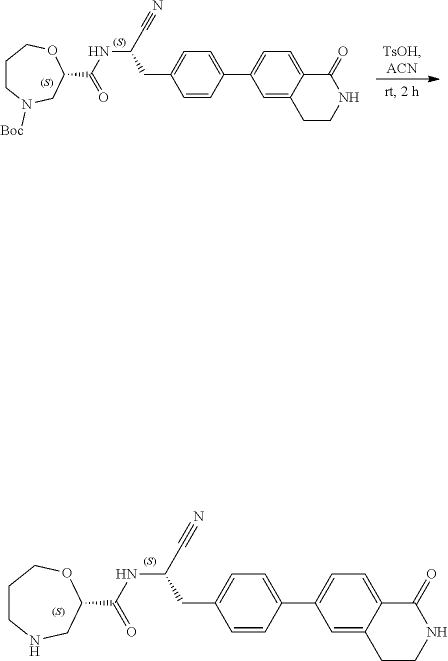

Into a 50 mL round-bottom flask were added tert-butyl (2S)-2-{[(1S)-1-cyano-2-[4-(1-oxo-3,4-dihydro-2H-isoquinolin-6-yl)phenyl]ethyl]carbamoyl}-1,4-oxazepane-4-carboxylate (100 mg, 0.19 mmol, 1 equiv), ACN (3 mL) and TsOH·H₂O (110 mg, 0.58 mmol, 3 equiv) at room temperature. The resulting mixture was stirred for 2 h at room temperature. The crude product was purified by Prep-HPLC with the following conditions (Prep-HPLC-013): Column, Atlantis Prep T3 OBD Column, 19*150 mm Sum; mobile phase, Water (0.05% NH₃·H₂O) and ACN (25% PhaseB up to 50% in 10 min) to afford Compound A2 (22.5 mg, 27.88%) as a white solid.

LCMS (ES, m/z): [M+H]⁺: 419. ¹H NMR (400 MHz, DMSO-d₆) δ 8.61 (d, J=8.5 Hz, 1H), 7.90 (d, J=8.2 Hz, 2H), 7.68 (d, J=7.9 Hz, 2H), 7.66-7.59 (m, 2H), 7.41 (d, J=7.9 Hz, 2H), 5.04 (q, J=8.2 Hz, 1H), 3.99 (dd, J=7.8, 3.6 Hz, 1H), 3.90-3.80 (m, 1H), 3.78-3.67 (m, 1H), 3.45-3.37 (m, 2H), 3.25-3.18 (m, 2H), 3.07-2.94 (m, 3H), 2.82-2.71 (m, 1H), 2.66-2.52 (m, 2H), 1.73 (s, 2H).

### Example 3: Synthesis of (2S)—N-[(1S)-1-cyano-2-{2-fluoro-4-[1-(1-methylpiperidin-4-yl)indazol-6-yl]phenyl}ethyl]-1,4-oxazepane-2-carboxamide (Compound A3)

### Step 1. Synthesis of tert-butyl (2S)-2-{[(1S)-1-cyano-2-{2-fluoro-4-[1-(1-methylpiperidin-4-yl)indazol-6-yl]phenyl}ethyl]carbamoyl}-1,4-oxazepane-4-carboxylate

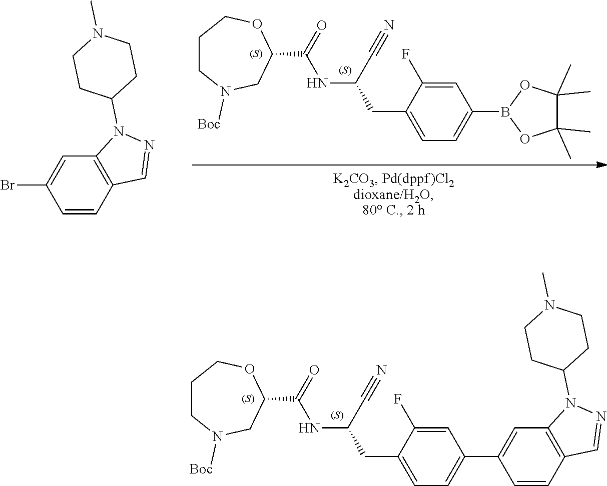

To a solution of tert-butyl (2S)-2-{[(1S)-1-cyano-2-[2-fluoro-4-(4,4,5,5-tetramethyl-1,3,2-dioxaborolan-2-yl)phenyl]ethyl]carbamoyl}-1,4-oxazepane-4-carboxylate (253 mg, 0.49 mmol, 1.2 equiv) and 6-bromo-1-(1-methylpiperidin-4-yl)indazole (120 mg, 0.41 mmol, 1 equiv) in dioxane (5 mL) and H₂O (0.5 mL) were added K₂CO₃ (113 mg, 0.82 mmol, 2 equiv) and Pd(dppf)Cl₂ (29.85 mg, 0.04 mmol, 0.1 equiv). After stirring for 2 h at 80° C. under a nitrogen atmosphere, the resulting mixture was concentrated under reduced pressure. The residue was purified by silica gel column chromatography, eluted with PE/THF (2:1) to afford tert-butyl (2S)-2-{[(1S)-1-cyano-2-{2-fluoro-4-[1-(1-methylpiperidin-4-yl)indazol-6-yl]phenyl}ethyl]carbamoyl}-1,4-oxazepane-4-carboxylate (100 mg, 40.54%) as a yellow oil. LCMS (ES, m/z): [M+H]⁺: 605.

### Step 2. Synthesis of (2S)—N-[(1S)-1-cyano-2-{2-fluoro-4-[1-(1-methylpiperidin-4-yl)indazol-6-yl]phenyl}ethyl]-1,4-oxazepane-2-carboxamide

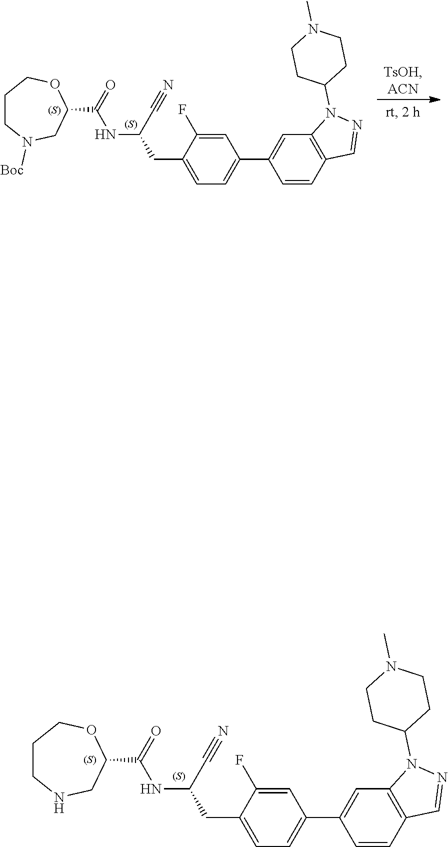

Into a 100 mL round-bottom flask were added tert-butyl (2S)-2-{[(1S)-1-cyano-2-{2-fluoro-4-[1-(1-methylpiperidin-4-yl)indazol-6-yl]phenyl}ethyl]carbamoyl}-1,4-oxazepane-4-carboxylate (100 mg, 0.165 mmol, 1 equiv), TsOH (85.43 mg, 0.495 mmol, 3 equiv) and ACN (3 mL) at room temperature. The resulting mixture was stirred for additional 2 h at room temperature. The reaction solution was purified by reversed-phase flash chromatography with the following conditions: C18-120 g column, mobile phase, MeCN in Water (0.05% NH₃·H₂O), 10% to 60% gradient in 10 min; detector, UV 254 nm. The fraction of the target was freezing dried, this resulted in Compound A3 (18 mg, 21.57%) as a white solid. LCMS (ES, m/z): [M+H]⁺: 505. ¹H NMR (400 MHz, DMSO-d6) δ 8.73 (d, J=8.5 Hz, 1H), 8.12-8.06 (m, 2H), 7.82 (d, J=8.4 Hz, 1H), 7.70 (d, J=11.7 Hz, 1H), 7.65 (d, J=7.9 Hz, 1H), 7.53-7.44 (m, 2H), 5.07 (q, J=8.2 Hz, 1H), 4.81-4.72 (m, 1H), 4.01 (dd, J=7.9, 3.6 Hz, 1H), 3.87 (dt, J=11.1, 5.2 Hz, 1H), 3.79-3.70 (m, 1H), 3.22 (dd, J=13.8, 8.5 Hz, 1H), 3.06 (dd, J=14.3, 3.7 Hz, 1H), 2.92 (d, J=6.8 Hz, 2H), 2.83-2.73 (m, 1H), 2.68-2.53 (m, 2H), 2.25 (s, 3H), 2.20-2.12 (m, 4H), 1.96-1.86 (m, 2H), 1.80-1.71 (m, 2H).

### Example 4: Synthesis of (2S)—N-[(1S)-1-cyano-2-[4-(2,3-dihydro-1-benzothiophen-5-yl)phenyl]ethyl]-1,4-oxazepane-2-carboxamide (Compound A4)

### Step 1. Synthesis of 1lambda6-benzothiophene-1,1-dione

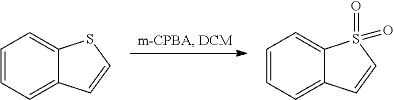 [SMILES: c1ccc2sccc2c1 ; O=S1(=O)C=Cc2ccccc21]

Into a 500 mL round-bottom flask were added benzothiophene (5.00 g, 37.25 mmol, 1.00 equiv) and DCM (200.00 mL) at room temperature. To the above mixture was added m-CPBA (16.07 g, 93.14 mmol, 2.50 equiv) in portions at 0° C. The resulting mixture was stirred for additional 2 h at room temperature. The reaction was quenched with sat. NaHCO₃ (aq.) at 0° C. The resulting mixture was extracted with CH₂Cl₂ (3×200 mL). The combined organic layers were dried over anhydrous Na₂SO₄. After filtration, the filtrate was concentrated under reduced pressure. This resulted in 1lambda6-benzothiophene-1,1-dione (5.50 g, 88.83%) as white solid. LCMS (ES) [M+1]⁺ m/z: 167.

### Step 2. Synthesis of 2,3-dihydro-1lambda6-benzothiophene-1,1-dione

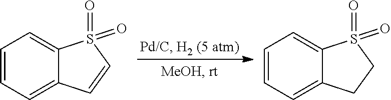 [SMILES: O=S1(=O)C=Cc2ccccc21 ; O=S1(=O)CCc2ccccc21]

Into a 250 mL pressure tank reactor were added 1lambda6-benzothiophene-1,1-dione (3.00 g, 18.05 mmol, 1.00 equiv), Pd/C (0.77 g, 7.22 mmol, 0.40 equiv) and MeOH (150.00 mL) at room temperature. The resulting mixture was stirred for 2 h at room temperature under hydrogen atmosphere. The resulting mixture was filtered, the filter cake was washed with MeOH (3×10 mL). The filtrate was concentrated under reduced pressure. This resulted in 2,3-dihydro-1lambda6-benzothiophene-1,1-dione (2.70 g, 88.92%) as off-white solid. LCMS (ES) [M+1]⁺ m/z: 169.

### Step 3. Synthesis of 2,3-dihydro-1-benzothiophen

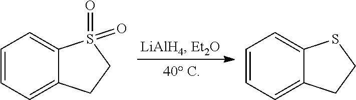 [SMILES: O=S1(=O)CCc2ccccc21 ; c1ccc2c(c1)CCS2]

Into a 500 mL 3-necked round-bottom flask were added LiAlH₄ (5.48 g, 144.45 mmol, 9.00 equiv) and Et₂O (130.00 mL) at room temperature. To the above mixture was added 2,3-dihydro-1lambda6-benzothiophene-1,1-dione (2.70 g, 16.05 mmol, 1.00 equiv) in Et₂O (130.00 mL) dropwise at 0° C. The resulting mixture was stirred for additional 3 h at 40° C. The reaction was quenched with Na₂SO₄·10H₂O at 0° C. The resulting mixture was filtered, the filter cake was washed with Et₂O (3×10 mL). The filtrate was concentrated under reduced pressure. The residue was purified by silica gel column chromatography, eluted with PE/EA (10:1) to afford 2,3-dihydro-1-benzothiophene (2.00 g, 91.48%) as colorless oil.

### Step 4. Synthesis of 5-bromo-2,3-dihydro-1-benzothiophene

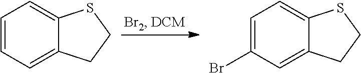 [SMILES: c1ccc2c(c1)CCS2 ; Brc1ccc2c(c1)CCS2]

Into a 100 mL round-bottom flask were added 2,3-dihydro-1-benzothiophene (800.00 mg, 5.87 mmol, 1.00 equiv) and DCM (15.00 mL) at room temperature. To the above mixture was added Br₂ (938.60 mg, 5.87 mmol, 1.00 equiv) dropwise at room temperature. The resulting mixture was stirred for additional 2 h at room temperature. The reaction was quenched with sat. NaHSO₃ (aq.) at 0° C. The resulting mixture was extracted with CH₂Cl₂ (3×20 mL). The combined organic layers were dried over anhydrous Na₂SO₄. After filtration, the filtrate was concentrated under reduced pressure. The residue was purified by silica gel column chromatography, eluted with PE/EA (10:1) to afford 5-bromo-2,3-dihydro-1-benzothiophene (900.00 mg, 71.24%) as Brown yellow oil.

### Step 5. Synthesis of tert-butyl (2S)-2-{[(1S)-1-cyano-2-[4-(2,3-dihydro-1-benzothiophen-5-yl)phenyl]ethyl]carbamoyl}-1,4-oxazepane-4-carboxylate

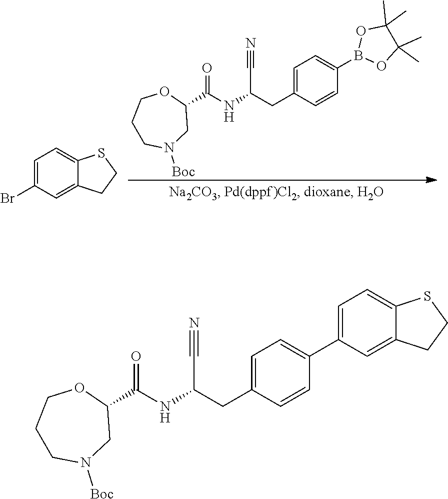 [SMILES: Brc1ccc2c(c1)CCS2 ; CC(C)(C)OC(=O)N1CCCO[C@H](C(=O)N[C@H](C#N)Cc2ccc(B3OC(C)(C)C(C)(C)O3)cc2)C1 ; CC(C)(C)OC(=O)N1CCCO[C@H](C(=O)N[C@H](C#N)Cc2ccc(-c3ccc4c(c3)CCS4)cc2)C1]

Into a 20 mL vial were added 5-bromo-2,3-dihydro-1-benzothiophene (64.61 mg, 0.30 mmol, 1.50 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (100.00 mg, 0.20 mmol, 1.00 equiv), Na₂CO₃ (42.44 mg, 0.40 mmol, 2.00 equiv), Pd(dppf)Cl₂ (14.65 mg, 0.02 mmol, 0.10 equiv), dioxane (5.00 mL) and H₂O (0.50 mL) at room temperature. The resulting mixture was stirred for 2 h at 100° C. under nitrogen atmosphere. The resulting mixture was concentrated under vacuum. The residue was purified by silica gel column chromatography, eluted with PE/EA (5:1) to afford tert-butyl (2S)-2-{[(1S)-1-cyano-2-[4-(2,3-dihydro-1-benzothiophen-5-yl)phenyl]ethyl]carbamoyl}-1,4-oxazepane-4-carboxylate (50.00 mg, 49.19%) as white solid. LCMS (ES) [M+1]⁺ m/z: 508.

### Step 6. Synthesis of (2S)—N-[(1S)-1-cyano-2-[4-(2,3-dihydro-1-benzothiophen-5-yl)phenyl]ethyl]-1,4-oxazepane-2-carboxamide

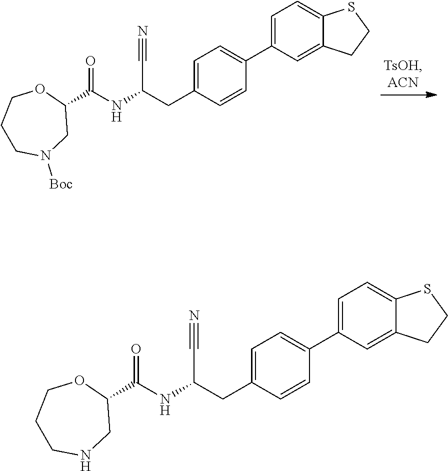 [SMILES: CC(C)(C)OC(=O)N1CCCO[C@H](C(=O)N[C@H](C#N)Cc2ccc(-c3ccc4c(c3)CCS4)cc2)C1 ; N#C[C@H](Cc1ccc(-c2ccc3c(c2)CCS3)cc1)NC(=O)[C@@H]1CNCCCO1]

Into a 20 mL vial were added tert-butyl (2S)-2-{[(1S)-1-cyano-2-[4-(2,3-dihydro-1-benzothiophen-5-yl)phenyl]ethyl]carbamoyl}-1,4-oxazepane-4-carboxylate (120.00 mg, 0.23 mmol, 1.00 equiv) and ACN (5.00 mL) at room temperature. To the above mixture was added TsOH (122.12 mg, 0.70 mmol, 3.00 equiv) in portions at 0° C. The resulting mixture was stirred for additional 2 h at room temperature. The residue was purified by reversed-phase flash chromatography with the following conditions: column, C18 silica gel; mobile phase, MeCN in Water (0.1% FA), 10% to 50% gradient in 10 min; detector, UV 254 nm. This resulted in Compound A4 (18.00 mg, 18.69%) as white solid. LCMS (ES) [M+1]⁺ m/z: 408. ¹H NMR (300 MHz, DMSO-d₆) δ 8.58 (d, J=8.5 Hz, 1H), 7.60-7.49 (m, 3H), 7.39-7.30 (m, 4H), 4.99 (m, 1H), 3.97 (dd, J=7.8, 3.6 Hz, 1H), 3.82 (dt, J=10.8, 5.1 Hz, 1H), 3.70 (ddd, J=12.1, 7.4, 4.3 Hz, 1H), 3.39 (dd, J=9.4, 6.8 Hz, 2H), 3.27 (d, J=7.7 Hz, 2H), 3.16 (dd, J=8.0, 2.9 Hz, 2H), 3.00 (dd, J=14.3, 3.7 Hz, 1H), 2.74 (dt, J=11.7, 5.6 Hz, 1H), 2.64-2.49 (m, 2H), 1.77-1.67 (m, 2H).

### Example 5: Synthesis (2S)—N-[(1S)-1-cyano-2-[4-(3,4-dihydro-2H-1-benzothiopyran-6-yl)phenyl]ethyl]-1,4-oxazepane-2-carboxamide (Compound A5)

### Step 1. Synthesis of 3-[(4-bromophenyl)sulfanyl]propanoic acid

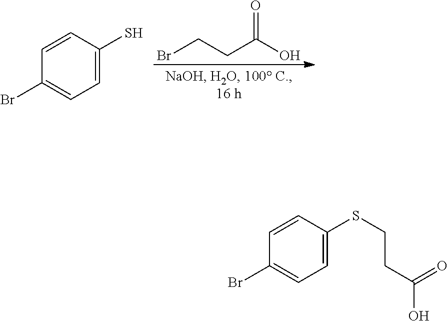 [SMILES: Sc1ccc(Br)cc1 ; O=C(O)CCBr ; O=C(O)CCSc1ccc(Br)cc1]

To a stirred solution of benzenethiol, 4-bromo- (7.7 g, 40.726 mmol, 1.0 equiv) and propanoic acid, 3-bromo- (10.59 g, 69.234 mmol, 1.7 equiv) in H₂O (100 mL) were added NaOH (3.91 g, 97.742 mmol, 2.4 equiv). The resulting mixture was stirred for 16 h at 100° C. The mixture was allowed to cool down to room temperature. The mixture was acidified to pH 5 with HCl (aq.) (1 MOL/L). The precipitated solids were collected by filtration and washed with water (2×20 mL). This resulted in 3-[(4-bromophenyl)sulfanyl]propanoic acid (9.7 g, 91.2%) as a white solid. LCMS (ES) [M-1]⁻ m/z: 259/261=1:1.

¹H NMR (300 MHz, DMSO-d6) δ 12.39 (s, 1H), 7.64-7.43 (m, 2H), 7.34-7.24 (m, 2H), 3.14 (t, J=7.0 Hz, 2H), 2.53 (t, J=7.0 Hz, 2H).

### Step 2. Synthesis of 6-bromo-2,3-dihydro-1-benzothiopyran-4-one

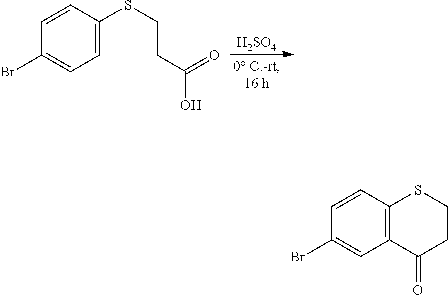 [SMILES: O=C(O)CCSc1ccc(Br)cc1 ; "H2SO4" ; O=C1CCSc2ccc(Br)cc21]

To a stirred sulfuric acid (50 mL) were added 3-[(4-bromophenyl)sulfanyl]propanoic acid (9.7 g, 37.146 mmol, 1.0 equiv) in portions at 0° C. under nitrogen atmosphere. The resulting mixture was stirred for 16 h at room temperature. The reaction mixture was poured into Water/Ice (300 mL). The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (20:1) to afford 6-bromo-2,3-dihydro-1-benzothiopyran-4-one (5.5 g, 60.9%) as a white solid. LCMS (ES) [M+1]m/z: 243.

¹H NMR (300 MHz, DMSO-d6) δ 8.01 (d, J=2.4 Hz, 1H), 7.64 (dd, J=8.5, 2.4 Hz, 1H), 7.36 (d, J=8.5 Hz, 1H), 3.39-3.27 (m, 2H), 2.96-2.85 (m, 2H).

### Step 3. Synthesis of 6-bromo-3,4-dihydro-2H-1-benzothiopyran

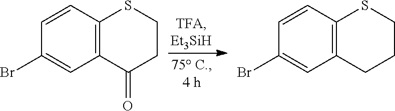 [SMILES: O=C1CCSc2ccc(Br)cc21 ; Brc1ccc2c(c1)CCCS2]

To a stirred solution of 6-bromo-2,3-dihydro-1-benzothiopyran-4-one (500 mg, 2.057 mmol, 1.0 equiv) in TFA (7 mL) was added Et₃SiH (478 mg, 4.114 mmol, 2.0 equiv). The resulting mixture was stirred for 4 h at 75° C. The mixture was allowed to cool down to room temperature. The resulting mixture was diluted with water (10 mL). The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (99:1) to afford 6-bromo-3,4-dihydro-2H-1-benzothiopyran (250 mg, 53.0%) as a light-yellow oil. ¹H NMR (400 MHz, DMSO-d6) δ 7.28 (dd, J=2.2, 1.1 Hz, 1H), 7.22 (dd, J=8.4, 2.3 Hz, 1H), 7.00 (d, J=8.4 Hz, 1H), 3.06-2.98 (m, 2H), 2.85-2.73 (m, 2H), 2.05-1.92 (m, 2H).

### Step 4. Synthesis of tert-butyl (2S)-2-{[(1S)-1-cyano-2-[4-(3,4-dihydro-2H-1-benzothiopyran-6-yl)phenyl]ethyl]carbamoyl}-1,4-oxazepane-4-carboxylate

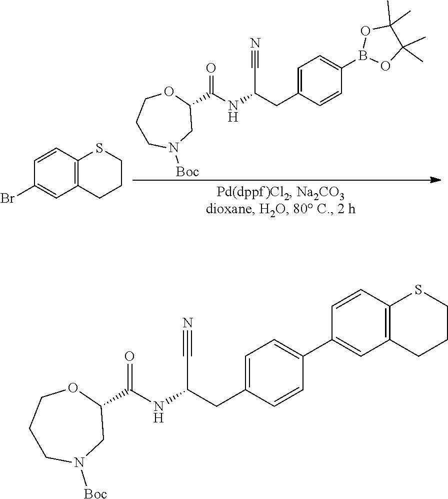

To a stirred solution of 6-bromo-3,4-dihydro-2H-1-benzothiopyran (65 mg, 0.286 mmol, 1.1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (130 mg, 0.260 mmol, 1.0 equiv) in dioxane (3 mL) and H₂O (0.3 mL) were added Na₂CO₃ (55 mg, 0.520 mmol, 2.0 equiv) and Pd(dppf)Cl₂ (19 mg, 0.026 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/EA (2:1) to afford tert-butyl (2S)-2-{[(1S)-1-cyano-2-[4-(3,4-dihydro-2H-1-benzothiopyran-6-yl)phenyl]ethyl]carbamoyl}-1,4-oxazepane-4-carboxylate (70 mg, 51.5%) as a light yellow solid. LCMS (ES) [M+1]⁺ m/z: 522.

### Step 5. Synthesis of (2S)—N-[(1S)-1-cyano-2-[4-(3,4-dihydro-2H-1-benzothiopyran-6-yl)phenyl]ethyl]-1,4-oxazepane-2-carboxamide

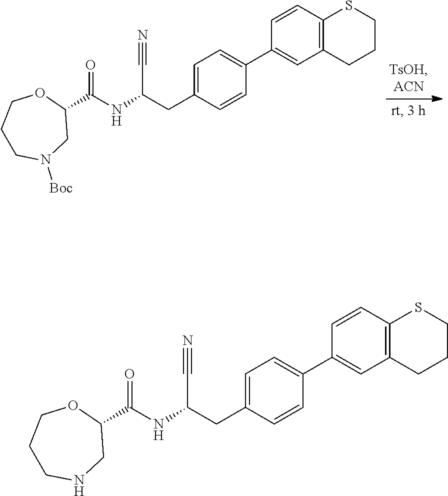 [SMILES: CC(C)(C)OC(=O)N1CCCO[C@H](C(=O)N[C@H](C#N)Cc2ccc(-c3ccc4c(c3)CCCS4)cc2)C1 ; N#C[C@H](Cc1ccc(-c2ccc3c(c2)CCCS3)cc1)NC(=O)[C@@H]1CNCCCO1]

To a stirred solution of tert-butyl (2S)-2-{[(1S)-1-cyano-2-[4-(3,4-dihydro-2H-1-benzothiopyran-6-yl)phenyl]ethyl]carbamoyl}-1,4-oxazepane-4-carboxylate (70 mg, 0.134 mmol, 1.0 equiv) in ACN (2 mL) was added TsOH (69 mg, 0.402 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (10 MMOL/L NH₄HCO₃) and ACN (10% PhaseB up to 80% in 20 min); Detector, UV 254 nm. This resulted in Compound A5 (18 mg, 31.8%) as a white solid. LCMS (ES) [M+1]⁺ m/z: 422. ¹H NMR (300 MHz, DMSO-d6) δ 8.62 (d, J=8.6 Hz, 1H), 7.62-7.53 (m, 2H), 7.40-7.30 (m, 4H), 7.11 (d, J=8.0 Hz, 1H), 5.01 (td, J=8.5, 7.2 Hz, 1H), 4.00 (dd, J=7.9, 3.7 Hz, 1H), 3.85 (ddd, J=12.3, 6.0, 4.6 Hz, 1H), 3.72 (ddd, J=12.2, 7.4, 4.3 Hz, 1H), 3.27-3.10 (m, 2H), 3.10-2.97 (m, 3H), 2.84 (t, J=6.1 Hz, 2H), 2.83-2.70 (m, 1H), 2.68-2.50 (m, 2H), 2.11-1.97 (m, 2H), 1.84-1.59 (m, 2H).

### Example 6: Synthesis of (2S)—N-[(1S)-1-cyano-2-(4-{1H-pyrrolo[2,3-c]pyridin-2-yl}phenyl)ethyl]-1,4-oxazepane-2-carboxamide (Compound A7)

### Step 1. Synthesis of tert-butyl pyrrolo[2,3-c]pyridine-1-carboxylate

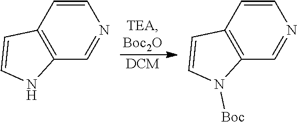

Into a 100 mL round-bottom flask were added 6-azaindole (1.00 g, 8.46 mmol, 1.00 equiv), di-tert-butyl dicarbonate (2.77 g, 12.69 mmol, 1.50 equiv), TEA (1.71 g, 16.93 mmol, 2.00 equiv) and DCM (20.00 mL) at room temperature. The resulting mixture was stirred for additional 3 h at room temperature. The reaction was quenched by the addition of Water/Ice (30 mL) at 0° C. The aqueous layer was extracted with EtOAc (3×30 mL). The resulting mixture was concentrated under reduced pressure. The residue was purified by silica gel column chromatography, eluted with PE/THF (5%) to afford tert-butyl pyrrolo[2,3-c]pyridine-1-carboxylate (1.8 g, 97.43%) as Brown yellow oil. LCMS (ES) [M+1]⁺ m/z: 219.

### Step 2. Synthesis of tert-butyl 2-iodopyrrolo[2,3-c]pyridine-1-carboxylate

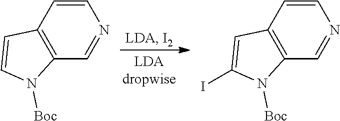 [SMILES: CC(C)(C)OC(=O)n1ccc2ccncc21 ; CC(C)(C)OC(=O)n1c(I)cc2ccncc21]

Into a 100 mL 3-necked round-bottom flask were added tert-butyl pyrrolo[2,3-c]pyridine-1-carboxylate (500.00 mg, 2.29 mmol, 1.00 equiv) and THF (10.00 mL) at room temperature. To the above mixture was added LDA (0.49 g, 4.58 mmol, 2.00 equiv) dropwise at −78° C. The resulting mixture was stirred for additional 1 h at −78° C. To the above mixture was added I₂ (0.70 g, 2.74 mmol, 1.20 equiv) dropwise at −78° C. The resulting mixture was stirred for additional 1 h at −78° C. The reaction was quenched with sat. NH₄Cl (aq.) at −40° C. The aqueous layer was extracted with EtOAc (3×30 mL). The resulting mixture was concentrated under reduced pressure. The residue was purified by silica gel column chromatography, eluted with PE/THF (10/1) to afford tert-butyl 2-iodopyrrolo[2,3-c]pyridine-1-carboxylate (750 mg, 95.13%) as yellow solid. LCMS (ES) [M+1]⁺ m/z: 345.

### Step 3. Synthesis of tert-butyl (2S)-2-{[(1S)-2-{4-[1-(tert-butoxycarbonyl)pyrrolo[2,3-c]pyridin-2-yl]phenyl}-1-cyanoethyl]carbamoyl}-1,4-oxazepane-4-carboxylate

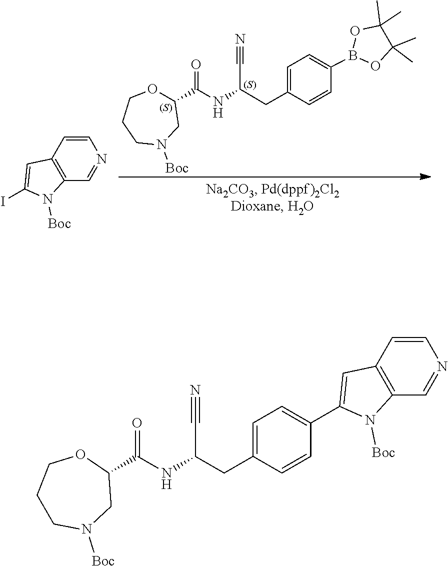

Into a 20 mL vial were added tert-butyl 2-iodopyrrolo[2,3-c]pyridine-1-carboxylate (68.91 mg, 0.20 mmol, 1.00 equiv), Na₂CO₃ (42.44 mg, 0.40 mmol, 2.00 equiv), Pd(dppf)Cl₂ (11.06 mg, 0.02 mmol, 0.10 equiv), dioxane (3.00 mL) and H₂O (0.30 mL) at room temperature. The resulting mixture was stirred for additional 2 h at 80° C. The resulting mixture was concentrated under reduced pressure. The residue was purified by silica gel column chromatography, eluted with PE/THF (1:1) to afford tert-butyl (2S)-2-{[(1S)-2-{4-[1-(tert-butoxycarbonyl)pyrrolo[2,3-c]pyridin-2-yl]phenyl}-1-cyanoethyl]carbamoyl}-1,4-oxazepane-4-carboxylate (95 mg, 80.46%) as colorless oil. LCMS (ES) [M+1]⁺ m/z: 590.

### Step 4. Synthesis of (2S)—N-[(1S)-1-cyano-2-(4-{1H-pyrrolo[2,3-c]pyridin-2-yl}phenyl)ethyl]-1,4-oxazepane-2-carboxamide

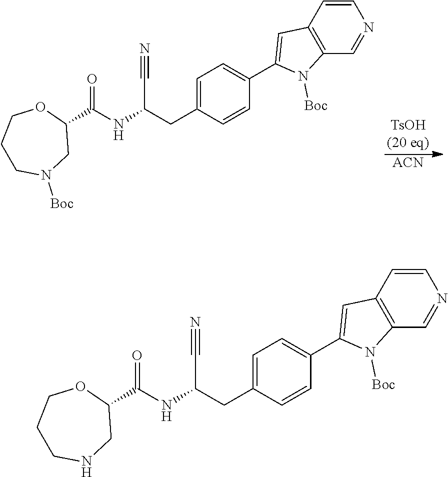 [SMILES: CC(C)(C)OC(=O)N1CCCO[C@H](C(=O)N[C@H](C#N)Cc2ccc(-c3cc4ccncc4n3C(=O)OC(C)(C)C)cc2)C1 ; "ACN" ; CC(C)(C)OC(=O)n1c(-c2ccc(C[C@@H](C#N)NC(=O)[C@@H]3CNCCCO3)cc2)cc2ccncc21]

Into a 8 mL vial were added tert-butyl (2S)-2-{[(1S)-2-{4-[1-(tert-butoxycarbonyl)pyrrolo[2,3-c]pyridin-2-yl]phenyl}-1-cyanoethyl]carbamoyl}-1,4-oxazepane-4-carboxylate (80.00 mg, 0.13 mmol, 1.00 equiv), ACN (4.00 mL) and TsOH (70.08 mg, 0.40 mmol, 3.00 equiv) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 2500 to 50% gradient in 10 min; detector, UV 254 nm. This resulted in Compound A7 (15.3 mg, 28.96%) as white solid. ¹H NMR (300 MHz, DMSO-d6) δ12.01 (s, 1H), 8.74 (s, 1H), 8.67 (d, J=8.6 Hz, 1H), 8.09 (d, J=5.5 Hz, 1H), 7.89 (d, J=7.8 Hz, 2H), 7.50 (d, J=5.5 Hz, 1H), 7.43 (d, J=8.0 Hz, 2H), 6.97 (s, 1H), 5.06 (d, J=8.1 Hz, 1H), 4.03 (d, J=5.2 Hz, 1H), 3.91-3.79 (m, 1H), 3.78-3.69 (m, 1H), 3.22 (d, J=8.4 Hz, 2H), 3.06 (d, J=10.9 Hz, 1H), 2.89-2.50 (m, 3H), 1.76 (in, 2H). L CMS (ES) [M+1]⁺ m/z: 390.

### Example 7: Synthesis of (2S)—N-[(1S)-1-cyano-2-(4-{1,3-dimethylpyrrolo[1,2-a]pyrazin-7-yl}phenyl)ethyl]-1,4-oxazepane-2-carboxamide (Compound A8)

### Step 1. Synthesis of 1-(4-bromo-1H-pyrrol-2-yl)ethanone

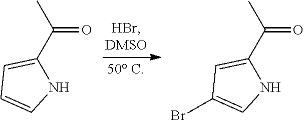

Into a 250 mL round-bottom flask were added 2-acetylpyrrole (4.50 g, 41.23 mmol, 1.00 equiv) and DMSO (45.00 mL) at room temperature. To the above mixture was added HBr in water (45.00 mL, 480) dropwise at 0° C. The resulting mixture was stirred for additional 2 h at room temperature. The mixture was allowed to cool down to room temperature. The mixture was neutralized to pH=7 with NaOH (4M). The resulting mixture was extracted with EtOAc (3×200 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (8:1) to afford 1-(4-bromo-1H-pyrrol-2-yl)ethanone (3.00 g, 38.69%) as yellow solid. LCMS (ES) [M+1]⁺ m/z: 188.

### Step 2. Synthesis of 1-(2-acetyl-4-bromopyrrol-1-yl)propan-2-one

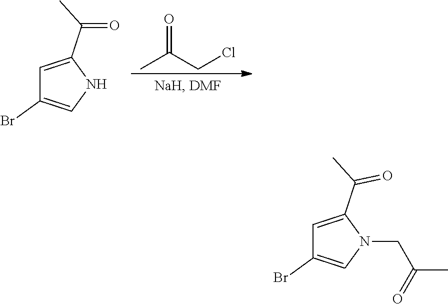

Into a 100 mL round-bottom flask were added 1-(4-bromo-1H-pyrrol-2-yl)ethanone (3.00 g, 15.95 mmol, 1.00 equiv) and DMF (40.00 mL) at room temperature. To the above mixture was added NaH (0.50 g, 20.74 mmol, 1.30 equiv) in portions at 0° C. The resulting mixture was stirred for additional 30 min at room temperature. To the above mixture was added 1-chloropropan-2-one (2.21 g, 23.93 mmol, 1.50 equiv) dropwise at 0° C. The resulting mixture was stirred for additional overnight at room temperature. The reaction was quenched with Water/Ice at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with THF/PE (15%) to afford 1-(2-acetyl-4-bromopyrrol-1-yl)propan-2-one (1.20 g, 30.81%) as yellow solid. LCMS (ES) [M+1]⁺ m/z: 244.

### Step 3. Synthesis of 7-bromo-1,3-dimethylpyrrolo[1,2-a]pyrazine

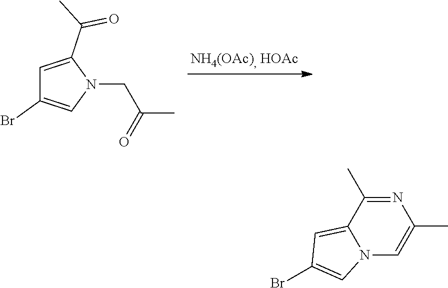

Into a 100 mL round-bottom flask were added 1-(2-acetyl-4-bromopyrrol-1-yl)propan-2-one (500.00 mg, 2.04 mmol, 1.00 equiv), NH₄OAc (3.16 g, 40.96 mmol, 20.00 equiv) and HOAc (20.00 mL) at room temperature. The resulting mixture was stirred for 2 h at 120° C. The resulting mixture was concentrated under reduced pressure. The mixture was neutralized to pH=7 with NaOH. The resulting mixture was extracted with EtOAc (3×20 mL). The combined organic layers were washed with brine (3×100 mL), dried over anhydrous Na₂SO₄. After filtration, the filtrate was concentrated under reduced pressure. The residue was purified by silica gel column chromatography, eluted with THF/PE (8.8%) to afford 7-bromo-1,3-dimethylpyrrolo[1,2-a]pyrazine (400.00 mg, 86.75%) as orange solid. LCMS (ES) [M+1]⁺ m/z: 225.

### Step 4. Synthesis of tert-butyl (2S)-2-{[(1S)-1-cyano-2-(4-{1,3-dimethylpyrrolo[1,2-a]pyrazin-7-yl}phenyl)ethyl]carbamoyl}-1,4-oxazepane-4-carboxylate

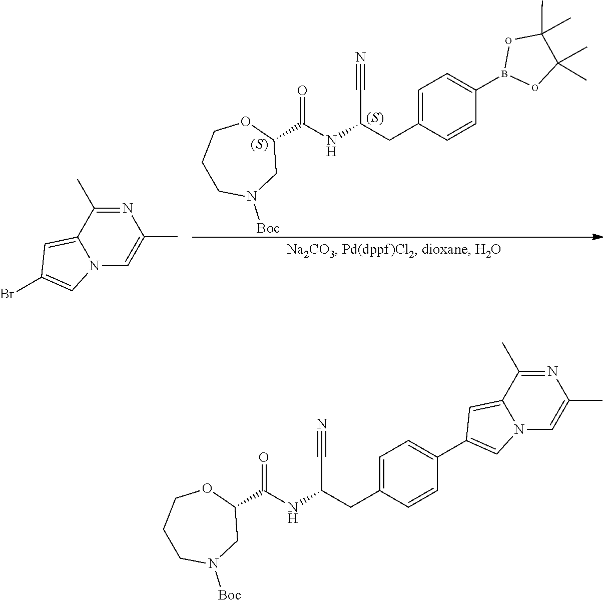

Into a 40 mL vial were added 7-bromo-1,3-dimethylpyrrolo[1,2-a]pyrazine (54.08 mg, 0.24 mmol, 1.20 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (100.00 mg, 0.20 mmol, 1.00 equiv), Na₂CO₃ (42.44 mg, 0.40 mmol, 2.00 equiv), Pd(dppf)Cl₂ (14.65 mg, 0.02 mmol, 0.10 equiv), dioxane (5.00 mL) and H₂O (0.50 mL) at room temperature. The resulting mixture was stirred for 2 h at 80° C. under nitrogen atmosphere. The resulting mixture was concentrated under reduced pressure. The residue was purified by silica gel column chromatography, eluted with THF/PE (32.5%) to afford tert-butyl (2S)-2-{[(1S)-1-cyano-2-(4-{1,3-dimethylpyrrolo[1,2-a]pyrazin-7-yl}phenyl)ethyl]carbamoyl}-1,4-oxazepane-4-carboxylate (90.00 mg, 86.83%) as light yellow solid. LCMS (ES) [M+1]⁺ m/z: 518.

### Step 5. Synthesis of (2S)—N-[(1S)-1-cyano-2-(4-{1,3-dimethylpyrrolo[1,2-a]pyrazin-7-yl}phenyl)ethyl]-1,4-oxazepane-2-carboxamide

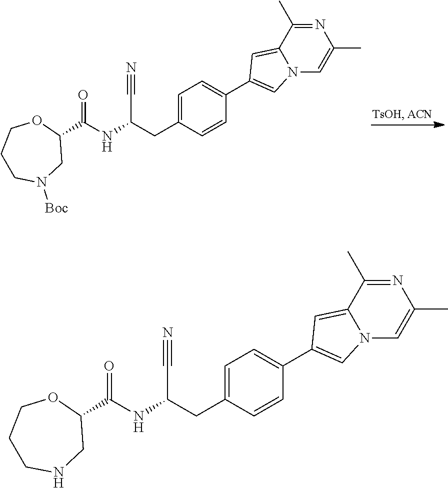 [SMILES: Cc1cn2cc(-c3ccc(C[C@@H](C#N)NC(=O)[C@@H]4CN(C(=O)OC(C)(C)C)CCCO4)cc3)cc2c(C)n1 ; Cc1cn2cc(-c3ccc(C[C@@H](C#N)NC(=O)[C@@H]4CNCCCO4)cc3)cc2c(C)n1]

Into a 8 mL vial were added tert-butyl (2S)-2-{[(1S)-1-cyano-2-(4-{1,3-dimethylpyrrolo[1,2-a]pyrazin-7-yl}phenyl)ethyl]carbamoyl}-1,4-oxazepane-4-carboxylate (80.00 mg, 0.15 mmol, 1.00 equiv), TsOH (79.84 mg, 0.46 mmol, 3.00 equiv) and ACN (3.00 mL) at room temperature. The final reaction mixture was irradiated with microwave radiation for 2 h at room temperature. The reaction solution was purified by Prep-HPLC with the following conditions XBridge Prep C18 OBD Column, 19*150 mm, 5 um; mobile phase, Water (10 mmol/L NH₄HCO₃) and ACN (30% Phase B up to 40% in 7 min); Detector, UV 254 nm. This resulted in Compound A8 (19.50 mg, 30.22%) as off-white solid. LCMS (ES) [M+1]⁺ m/z: 418. ¹H NMR (300 MHz, DMSO-d6) δ 8.62 (d, J=8.6 Hz, 1H), 8.03 (d, J=1.6 Hz, 1H), 7.91 (s, 1H), 7.71 (d, J=8.1 Hz, 2H), 7.33 (d, J=8.1 Hz, 2H), 7.18 (t, J=1.3 Hz, 1H), 5.01 (m, J=8.3 Hz, 1H), 4.01 (dd, J=8.0, 3.7 Hz, 1H), 3.93-3.80 (m, 1H), 3.73 (ddd, J=12.1, 7.4, 4.3 Hz, 1H), 3.21-3.13 (m, 2H), 3.04 (dd, J=14.3, 3.7 Hz, 1H), 2.79 (dd, J=12.9, 6.1 Hz, 1H), 2.69-2.59 (m, 1H), 2.57 (s, 3H), 2.54 (d, J=6.2 Hz, 1H), 2.28 (d, J=1.0 Hz, 3H), 1.79-1.69 (m, 2H).

### Example 8: Synthesis of (2S)—N-[(1S)-1-cyano-2-(4-{1H,2H,3H,5H-pyrido[2,3-e][1,4]oxazepin-7-yl}phenyl)ethyl]-1,4-oxazepane-2-carboxamide (Compound A9)

### Step 1. Synthesis of (2-amino-5-bromopyridin-3-yl)methanol hydrobromide

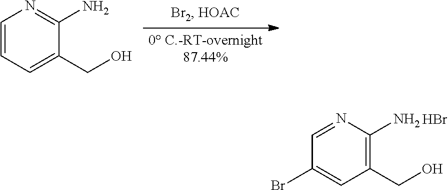 [SMILES: Nc1ncccc1CO ; Nc1ncc(Br)cc1CO ; Br]

Into a 250 mL 3-necked round-bottom flask were added (2-aminopyridin-3-yl)methanol (4.00 g, 32.22 mmol, 1.00 equiv) and HOAC (52.00 mL). To the above mixture was added Br₂ (5.20 g, 32.54 mmol, 1.01 equiv) dropwise at 0° C. The resulting mixture was stirred for additional overnight at room temperature. The mixture was allowed to cool down to room temperature. The precipitated solids were collected by filtration and washed with Et₂O (3×10 mL). The filtrate was concentrated under reduced pressure. This resulted in (2-amino-5-bromopyridin-3-yl)methanol hydrobromide (8.00 g, 87.44%) as a yellow solid. LCMS (ES, m z): [M-HBr+H]⁺: 203.

### Step 2. Synthesis of 5-bromo-3-(bromomethyl)pyridin-2-amine hydrobromide

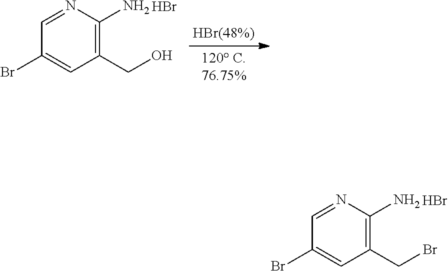 [SMILES: Nc1ncc(Br)cc1CO ; Br ; Nc1ncc(Br)cc1CBr ; Br]

Into a 250 mL round-bottom flask were added (2-amino-5-bromopyridin-3-yl)methanol hydrobromide (8.00 g, 28.17 mmol, 1.00 equiv) and HBr in water (80.00 mL, 48%). The resulting mixture was stirred for overnight at 120° C. The mixture was allowed to cool down to room temperature. The precipitated solids were collected by filtration and washed with EA (3×5 mL). This resulted in 5-bromo-3-(bromomethyl)pyridin-2-amine hydrobromide (7.50 g, 76.75%) as colorless crystal. LCMS (ES, m/z): [M-HBr+H]⁺: 265.

### Step 3. Synthesis of ethyl 2-[(2-amino-5-bromopyridin-3-yl)methoxy]acetate

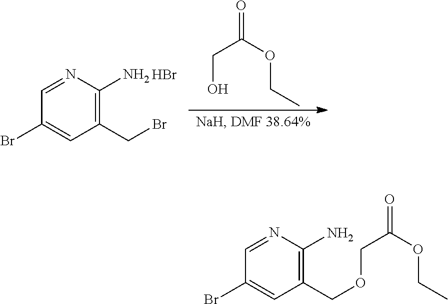

Into a 250 mL 3-necked round-bottom flask were added ethyl 2-hydroxyacetate (2.05 g, 19.69 mmol, 1.00 equiv) and DMF (50.00 mL). To the above mixture was added NaH (1.77 g, 29.54 mmol, 1.50 equiv, 40%) in portions at 0° C. The resulting mixture was stirred for additional 30 min at 0° C. To the above mixture was added 5-bromo-3-(bromomethyl)pyridin-2-amine hydrobromide (7.51 g, 21.66 mmol, 1.10 equiv) in portions at 0° C. The resulting mixture was stirred for additional 1 h at 0° C. The reaction was quenched with Water/Ice at 0° C. The resulting mixture was extracted with EA (3×50 mL). The combined organic layers were washed with brine (3×100 mL), dried over anhydrous Na₂SO₄. After filtration, the filtrate was concentrated under reduced pressure. The residue was purified by silica gel column chromatography, eluted with THF/PE (12%) to afford ethyl 2-[(2-amino-5-bromopyridin-3-yl)methoxy]acetate (2.20 g, 38.64%) as a yellow solid. LCMS (ES, m/z): [M+H]⁺: 289.

### Step 4. Synthesis of methyl 7-bromo-1H,3H,5H-pyrido[2,3-e][1,4]oxazepin-2-one

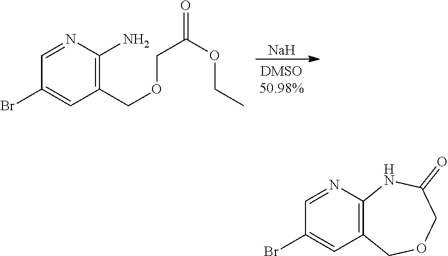

Into a 20 mL round-bottom flask were added ethyl 2-[(2-amino-5-bromopyridin-3-yl)methoxy]acetate (700.00 mg, 2.421 mmol, 1.00 equiv) and DMSO (6.00 mL). To the above mixture was added NaH (145.25 mg, 2.421 mmol, 1.00 equiv, 40%) in portions at 0° C. The resulting mixture was stirred for additional 5 h at room temperature. The reaction was quenched with Water/Ice. The precipitated solids were collected by filtration and washed with water/Ice (3×3 mL). The resulting mixture was concentrated under reduced pressure. This resulted in 7-bromo-1H,3H,5H-pyrido[2,3-e][1,4]oxazepin-2-one (300.00 mg, 50.98%) as an off-white solid. LCMS (ES, m/z): [M+H]⁺: 243.

### Step 5. Synthesis of 7-bromo-1H,2H,3H,5H-pyrido[2,3-e][1,4]oxazepane

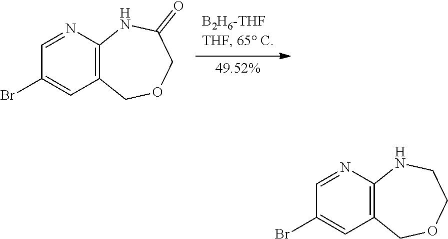 [SMILES: O=C1COCc2cc(Br)cnc2N1 ; Brc1cnc2c(c1)COCCN2]

Into a 50 mL vial were added 7-bromo-1H,3H,5H-pyrido[2,3-e][1,4]oxazepin-2-one (300.00 mg, 1.234 mmol, 1.00 equiv) and THF (12.00 mL). To the above mixture was added BH₃-THF (0.86 mL, 9.008 mmol, 7.30 equiv) dropwise at 0° C. The resulting mixture was stirred for additional overnight at 65° C. The mixture was allowed to cool down to room temperature. The reaction was quenched with Water/Ice (1 mL). The resulting mixture was concentrated under reduced pressure. To the above mixture was added NaOH (2M) (5 mL) and MeOH (10 mL). The resulting mixture was stirred for additional 4 h at 65° C. The resulting mixture was concentrated under reduced pressure. The resulting mixture was extracted with EtOAc (3×15 mL). The combined organic layers were dried over anhydrous Na₂SO₄. After filtration, the filtrate was concentrated under reduced pressure. The residue was purified by silica gel column chromatography, eluted with THF/PE (12%) to afford 7-bromo-1H,2H,3H,5H-pyrido[2,3-e][1,4]oxazepine (140.00 mg, 49.52%) as light yellow solid. LCMS (ES, m/z): [M+H]⁺: 229.

### Step 6. Synthesis of (2S)-2-{[(1S)-1-cyano-2-(4-{1H,2H,3H,5H-pyrido[2,3-e][1,4]oxazepin-7-yl}phenyl)ethyl]carbamoyl}-1,4-oxazepane-4-carboxylate

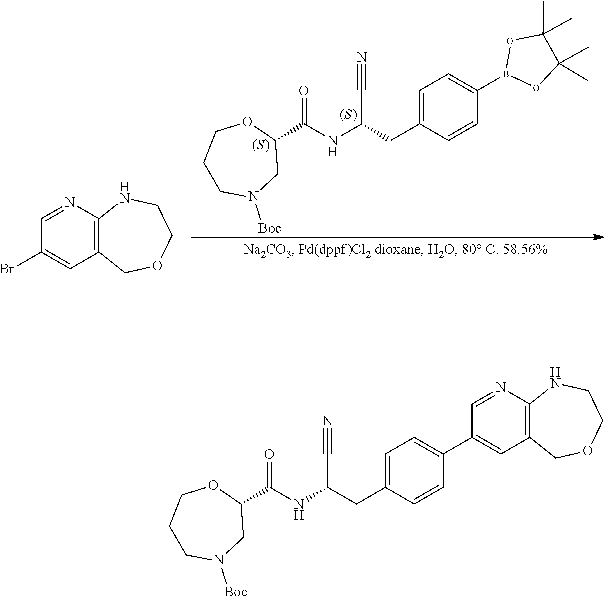

Into a 10 mL vial were added 7-bromo-1H,2H,3H,5H-pyrido[2,3-e][1,4]oxazepine (120.00 mg, 0.52 mmol, 1.00 equiv), dioxane (5.00 mL), H₂O (0.50 mL), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (313.94 mg, 0.63 mmol, 1.20 equiv), Na₂CO₃ (111.04 mg, 1.05 mmol, 2.00 equiv) and Pd(dppf)Cl₂ (38.33 mg, 0.05 mmol, 0.10 equiv). The resulting mixture was stirred for 2 h at 80° C. under nitrogen atmosphere. The mixture was allowed to cool down to room temperature. The residue was purified by silica gel column chromatography, eluted with THF/PE (44%) to afford tert-butyl (2S)-2-{[(1S)-1-cyano-2-(4-{1H,2H,3H,5H-pyrido[2,3-e][1,4]oxazepin-7-yl}phenyl)ethyl]carbamoyl}-1,4-oxazepane-4-carboxylate (160.00 mg, 58.56%) as light yellow solid. LCMS (ES, m/z): [M+H]⁺: 522.

### Step 7. Synthesis of (2S)—N-[(1S)-1-cyano-2-(4-{1H,2H,3H,5H-pyrido[2,3-e]|1,4]oxazepin-7-yl}phenyl)ethyl]-1,4-oxazepane-2-carboxamide

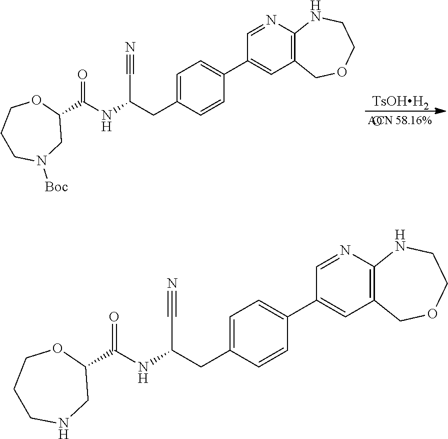

Into a 8 mL vial were added tert-butyl (2S)-2-{[(1S)-1-cyano-2-(4-{1H,2H,3H,5H-pyrido[2,3-e][1,4]oxazepin-7-yl}phenyl)ethyl]carbamoyl}-1,4-oxazepane-4-carboxylate (140.00 mg, 0.27 mmol, 1.00 equiv), ACN (4.00 mL) and TsOH-H₂O (153.15 mg, 0.80 mmol, 3.00 equiv). The resulting mixture was stirred for 2 h at room temperature. The reaction solution was purified by Prep-HPLC with the following conditions XBridge Prep C18 OBD Column, 19*150 mm, 5 um; mobile phase, Water (0.05% NH₃·H₂O) and ACN (35% Phase B up to 55% in 8.8 min); Detector, UV 254 nm. This resulted in Compound A9 (65.80 mg, 58.16%) as a white solid. LCMS (ES, m/z): [M+H]⁺: 422. ¹H NMR (300 MHz, DMSO-d₆) δ 8.60 (d, J=8.5 Hz, 1H), 8.31 (d, J=2.3 Hz, 1H), 7.80 (d, J=2.4 Hz, 1H), 7.64-7.54 (m, 2H), 7.34 (d, J=8.2 Hz, 2H), 6.44 (d, J=3.4 Hz, 1H), 5.01 (q, J=8.2 Hz, 1H), 4.55 (s, 2H), 3.99 (dd, J=7.8, 3.7 Hz, 1H), 3.85 (ddd, J=10.7, 6.0, 4.6 Hz, 1H), 3.78-3.64 (m, 3H), 3.17 (dq, J=6.8, 3.5, 2.9 Hz, 4H), 3.02 (dd, J=14.3, 3.7 Hz, 1H), 2.76 (dt, J=12.0, 5.4 Hz, 1H), 2.67-2.51 (m, 2H), 1.81-1.61 (m, 2H).

### Example 9: Synthesis of (2S)—N-[(1S)-1-cyano-2-[4-(5,6,7,8-tetrahydro-1,5-naphthyridin-2-yl)phenyl]ethyl]-1,4-oxazepane-2-carboxamide (Compound A10)

### Step 1. Synthesis of tert-butyl (2S)-2-{[(1S)-1-cyano-2-[4-(5,6,7,8-tetrahydro-1,5-naphthyridin-2-yl)phenyl]ethyl]carbamoyl}-1,4-oxazepane-4-carboxylate

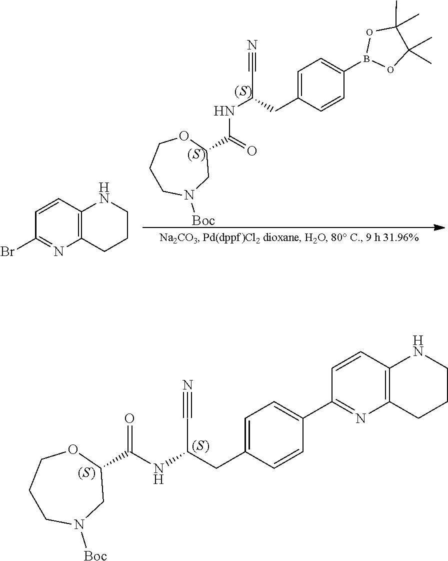

Into a 8 mL vial were added 2-bromo-5,6,7,8-tetrahydro-1,5-naphthyridine (120.00 mg, 0.56 mmol, 1.00 equiv), dioxane (5.00 mL), H₂O (0.50 mL), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (337.51 mg, 0.68 mmol, 1.20 equiv), Na₂CO₃ (119.38 mg, 1.13 mmol, 2.00 equiv) and Pd(dppf)Cl₂ (41.21 mg, 0.06 mmol, 0.10 equiv). The resulting mixture was stirred for 3 h at 80° C. under nitrogen atmosphere. The mixture was allowed to cool down to room temperature. The resulting mixture was concentrated under reduced pressure. The residue was purified by silica gel column chromatography, eluted with THF/PE (29%) to afford tert-butyl (2S)-2-{[(1S)-1-cyano-2-[4-(5,6,7,8-tetrahydro-1,5-naphthyridin-2-yl)phenyl]ethyl]carbamoyl}-1,4-oxazepane-4-carboxylate (140.00 mg, 31.96%) as light yellow solid. LCMS (ES, m/z): [M+H]⁺: 506.

### Step 2. Synthesis of (2S)—N-[(1S)-1-cyano-2-[4-(5,6,7,8-tetrahydro-1,5-naphthyridin-2-yl)phenyl]ethyl]-1,4-oxazepane-2-carboxamide

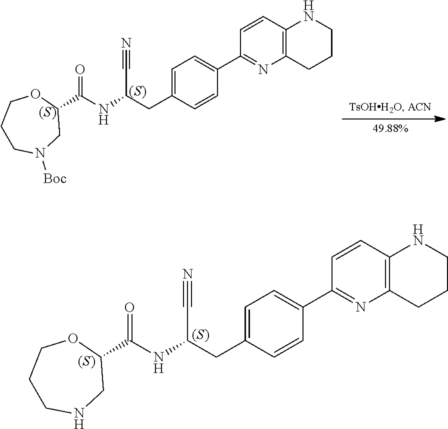

Into a 8 mL vial were added tert-butyl (2S)-2-{[(1S)-1-cyano-2-[4-(5,6,7,8-tetrahydro-1,5-naphthyridin-2-yl)phenyl]ethyl]carbamoyl}-1,4-oxazepane-4-carboxylate (100.00 mg, 0.13 mmol, 1.00 equiv, 65%), ACN (2.00 mL) and TsOH-H₂O (73.36 mg, 0.39 mmol, 3.00 equiv). The resulting mixture was stirred for 3 h at room temperature. The reaction solution was purified by reversed-phase flash chromatography with the following conditions: C18-120 g column, mobile phase, MeCN in Water (0.1% NH₃·H₂O), 20% to 50% gradient in 8 min; detector, UV 254 nm. The fraction of the target was freezing dried, this resulted in Compound A10 (26.00 mg, 49.88%) as white solid. LCMS (ES, m/z): [M+H]⁺: 406. ¹H NMR (300 MHz, DMSO-d₆) δ 8.59 (d, J=8.5 Hz, 1H), 7.88-7.79 (m, 2H), 7.46 (d, J=8.5 Hz, 1H), 7.28 (d, J=8.1 Hz, 2H), 6.83 (d, J=8.4 Hz, 1H), 6.01 (s, 1H), 4.98 (q, J=8.2 Hz, 1H), 3.99 (dd, J=8.0, 3.7 Hz, 1H), 3.86 (dt, J=10.7, 5.3 Hz, 1H), 3.79-3.65 (m, 1H), 3.26-3.10 (m, 4H), 3.03 (dd, J=14.3, 3.7 Hz, 1H), 2.84 (t, J=6.4 Hz, 2H), 2.75 (dd, J=12.7, 5.9 Hz, 1H), 2.69-2.51 (m, 2H), 1.92 (d, J=6.7 Hz, 2H), 1.85-1.65 (m, 2H).

### Example 10: Synthesis of (2S)—N-[(1S)-1-cyano-2-[4-(5,6,7,8-tetrahydroquinoxalin-2-yl)phenyl]ethyl]-1,4-oxazepane-2-carboxamide (Compound A11)

### Step 1. Synthesis of 5,6,7,8-tetrahydroquinoxalin-1-ium-1-olate

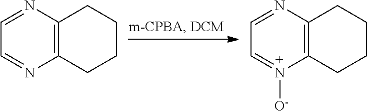

Into a 250 mL 3-necked round-bottom flask were added 5,6,7,8-tetrahydroquinoxaline (2.00 g, 14.90 mmol, 1.00 equiv) and DCM (140.00 mL) at room temperature. To the above mixture was added m-CPBA (3.09 g, 17.88 mmol, 1.20 equiv) in portions 0° C. The resulting mixture was stirred for additional overnight at room temperature. The mixture was neutralized to pH=8 with NaOH (1M). The resulting mixture was extracted with CH₂Cl₂ (3×200 mL). The combined organic layers were washed with brine (3×200 mL), dried over anhydrous Na₂SO₄. After filtration, the filtrate was concentrated under reduced pressure. The residue was purified by silica gel column chromatography, eluted with THF/PE (37%) to afford 5,6,7,8-tetrahydroquinoxalin-1-ium-1-olate (1.50 g, 67.01%) as light-yellow solid. LCMS (ES) [M+1]⁺ m/z: 151.

### Step 2. Synthesis of 2-bromo-5,6,7,8-tetrahydroquinoxaline

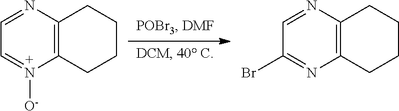

Into a 100 mL 3-necked round-bottom flask were added 5,6,7,8-tetrahydroquinoxalin-1-ium-1-olate (1.00 g, 6.65 mmol, 1.00 equiv) and DCM (35.00 mL) at room temperature. To the above mixture was added POBr₃ (5.73 g, 19.97 mmol, 3.00 equiv) in portions at 0° C. To the above mixture was added DMF (0.73 g, 9.98 mmol, 1.50 equiv) dropwise at 0° C. The resulting mixture was stirred for overnight at 40° C. under argon atmosphere. The mixture was neutralized to pH=7 with saturated Na₂CO₃ (aq.). The resulting mixture was extracted with CH₂Cl₂ (3×50 mL). The combined organic layers were washed with brine (3×200 mL), dried over anhydrous Na₂SO₄. After filtration, the filtrate was concentrated under reduced pressure. The residue was purified by silica gel column chromatography, eluted with THF/PE (15%) to afford 2-bromo-5,6,7,8-tetrahydroquinoxaline (500.00 mg, 35.24%) as yellow oil. LCMS (ES) [M+1]⁺ m/z: 213.

### Step 3. Synthesis of tert-butyl (2S)-2-{[(1S)-1-cyano-2-[4-(5,6,7,8-tetrahydroquinoxalin-2-yl)phenyl]ethyl]carbamoyl}-1,4-oxazepane-4-carboxylate

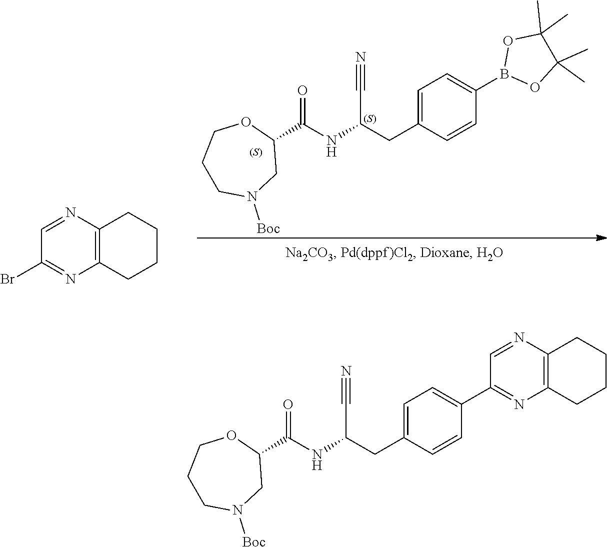

Into a 40 mL vial were added 2-bromo-5,6,7,8-tetrahydroquinoxaline (64.04 mg, 0.30 mmol, 1.20 equiv), (2S)—N-[(1S)-1-cyano-2-[4-(4,4,5,5-tetramethyl-1,3,2-dioxaborolan-2-yl)phenyl]ethyl]-1,4-oxazepane-2-carboxamide (100.00 mg, 0.25 mmol, 1.00 equiv), Na₂CO₃ (53.09 mg, 0.50 mmol, 2.00 equiv), Pd(dppf)Cl₂ (18.32 mg, 0.02 mmol, 0.10 equiv), dioxane (5.00 mL) and H₂O (0.50 mL) at room temperature. The resulting mixture was stirred for 2 h at 80° C. under nitrogen atmosphere. The resulting mixture was concentrated under reduced pressure. The residue was purified by silica gel column chromatography, eluted with THF/PE (25%) to afford tert-butyl (2S)-2-{[(1S)-1-cyano-2-[4-(5,6,7,8-tetrahydroquinoxalin-2-yl)phenyl]ethyl]carbamoyl}-1,4-oxazepane-4-carboxylate (90.00 mg, 71.08%) as off-white solid. LCMS (ES) [M+1]⁺ m/z: 506.

### Step 4. Synthesis of (2S)—N-[(1S)-1-cyano-2-[4-(5,6,7,8-tetrahydroquinoxalin-2-yl)phenyl]ethyl]-1,4-oxazepane-2-carboxamide

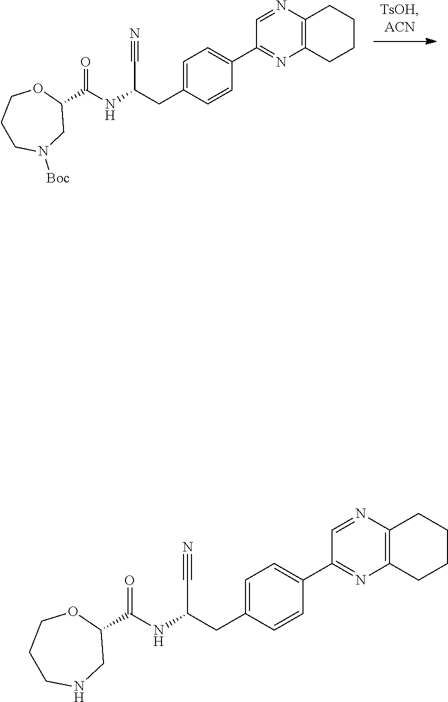 [SMILES: CC(C)(C)OC(=O)N1CCCO[C@H](C(=O)N[C@H](C#N)Cc2ccc(-c3cnc4c(n3)CCCC4)cc2)C1 ; N#C[C@H](Cc1ccc(-c2cnc3c(n2)CCCC3)cc1)NC(=O)[C@@H]1CNCCCO1]

Into a 8 mL vial were added tert-butyl (2S)-2-{[(1S)-1-cyano-2-[4-(5,6,7,8-tetrahydroquinoxalin-2-yl)phenyl]ethyl]carbamoyl}-1,4-oxazepane-4-carboxylate (80.00 mg, 0.15 mmol, 1.00 equiv), TsOH (81.74 mg, 0.47 mmol, 3.00 equiv) and ACN (4.00 mL) at room temperature. The resulting mixture was stirred for 2 h at room temperature. The reaction solution was purified by Prep-HPLC with the following conditions XBridge Prep C18 OBD Column, 19*150 mm, 5 um; mobile phase, Water (0.05% NH₃—H₂O) and ACN (25% Phase B up to 40% in 8 min); Detector, UV 254 nm. This resulted in Compound A11 (17.7 mg, 27.59%) as white solid. LCMS (ES) [M+1]⁺ m/z: 406. ¹H NMR (300 MHz, DMSO-d₆) δ 8.92 (s, 1H), 8.62 (d, J=8.6 Hz, 1H), 8.01 (d, J=8.2 Hz, 2H), 7.40 (d, J=8.0 Hz, 2H), 5.03 (q, J=8.2 Hz, 1H), 3.97 (dd, J=7.9, 3.6 Hz, 1H), 3.83 (dt, J=10.7, 5.1 Hz, 1H), 3.70 (ddd, J=12.1, 7.4, 4.2 Hz, 1H), 3.22-3.19 (m, 2H), 3.00 (dd, J=14.3, 3.7 Hz, 1H), 2.92-2.89 (m, 4H), 2.82-2.71 (m, 1H), 2.65-2.55 (m, 2H), 1.87 (p, J=3.2 Hz, 4H), 1.71 (t, J=5.9 Hz, 2H).

### Example 11: Synthesis of (2S)—N-[(1S)-1-cyano-2-(4-{1-oxo-2H,3H,4H,5H-pyrrolo[1,2-a][1,4]diazepin-8-yl}phenyl)ethyl]-1,4-oxazepane-2-carboxamide (Compound A12)

### Step 1. Synthesis of methyl 4-bromo-1-{3-[(tert-butoxycarbonyl)amino]propyl}pyrrole-2-carboxylate

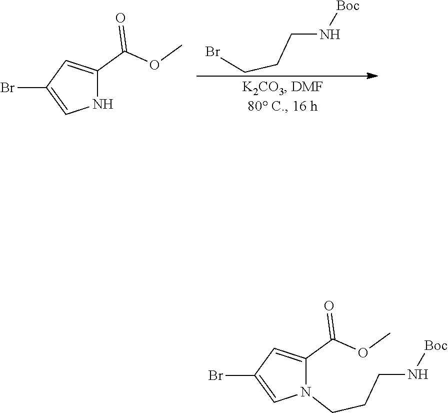

To solution of methyl 4-bromo-1H-pyrrole-2-carboxylate (0.6 g, 2.94 mmol, 1.0 equiv) in dimethylformamide (3.6 mL), was added tert-butyl N-(3-bromopropyl)carbamate (1.05 g, 4.41 mmol, 1.5 equiv) and K₂CO₃ (0.61 g, 4.41 mmol, 1.5 equiv). The resulting mixture was stirred for 16 h at 80° C. under N₂ atmosphere. The reaction was cooled to room temperature, the resulting mixture was extracted with EA (2×50 mL). The combined organic layers were washed with brine (50 mL), dried over anhydrous Na₂SO₄. After filtration, the filtrate was concentrated under reduced pressure. The residue was purified by silica gel column chromatography, eluted with EA in PE (Eluting with 0%˜40%) to afford methyl 4-bromo-1-{3-[(tert-butoxycarbonyl)amino]propyl}pyrrole-2-carboxylate (0.95 g, 89%) as a yellow solid. LCMS (ES, m/z): [M+H]⁺: 361.

### Step 2. Synthesis of methyl 1-(3-aminopropyl)-4-bromopyrrole-2-carboxylate

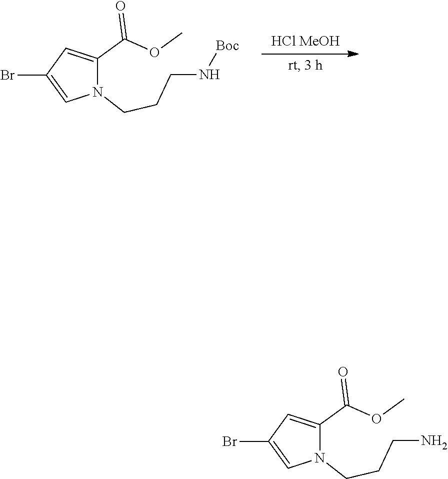 [SMILES: COC(=O)c1cc(Br)cn1CCCNC(=O)OC(C)(C)C ; COC(=O)c1cc(Br)cn1CCCN ; "HCl MeOH"]

To solution of methyl 4-bromo-1-{3-[(tert-butoxycarbonyl)amino]propyl}pyrrole-2-carboxylate (0.9 g, 2.49 mmol, 1.0 equiv) in methanol (5 mL) and HCl (c) (0.1 mL). The resulting mixture was stirred for 3 h at room temperature. The resulting mixture was concentrated under vacuum. The residue was adjusted to pH=8 with aqueous NaHCO₃ (30 mL), extracted DCM (2×50 mL). The combined organic layer was washed with brine (50 mL), dried over anhydrous Na₂SO₄. After filtration, the filtrate was concentrated under reduced pressure to afford methyl 1-(3-aminopropyl)-4-bromopyrrole-2-carboxylate (0.6 g, 92%) as a yellow solid and used to the next step without further purification. LCMS (ES, m/z): [M+H]⁺:261.

### Step 3. Synthesis of 8-bromo-2H,3H,4H,5H-pyrrolo[1,2-a][1,4]diazepin-1-one

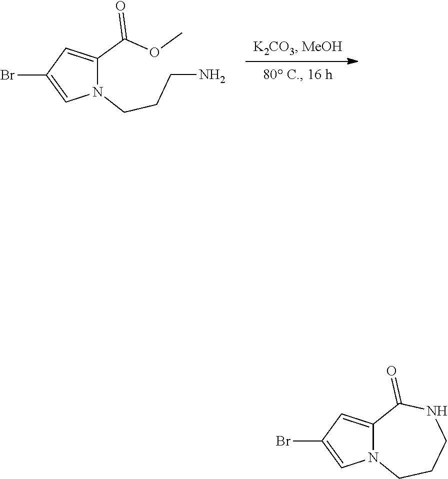

To a solution of methyl 1-(3-aminopropyl)-4-bromopyrrole-2-carboxylate (0.6 g, 2.29 mmol, 1.0 equiv) in methanol (6 mL), K₂CO₃ (0.95 g, 6.90 mmol, 3.0 equiv) was added at room temperature. The resulting mixture was stirred for 16 h at 80° C. The reaction was cooled to room temperature, then the K₂CO₃ was filtered off and the filtrate was concentrated under reduced pressure. The residue was purified by silica gel column chromatography, eluted with EA in PE (50%˜100%) to afford 8-bromo-2H,3H,4H,5H-pyrrolo[1,2-a][1,4]diazepin-1-one (0.5 g, 94%) as a yellow solid. LCMS (ES, m/z): [M+H]⁺: 229.

### Step 4. Synthesis of tert-butyl (2S)-2-{[(1S)-1-cyano-2-(4-{1-oxo-2H,3H,4H,5H-pyrrolo[1,2-a][1,4]diazepin-8-yl}phenyl)ethyl]carbamoyl}-1,4-oxazepane-4-carboxylate

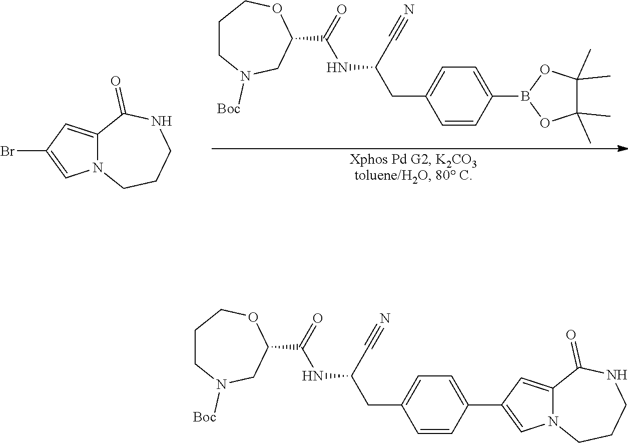 [SMILES: O=C1NCCCn2cc(Br)cc21 ; CC(C)(C)OC(=O)N1CCCO[C@H](C(=O)N[C@H](C#N)Cc2ccc(B3OC(C)(C)C(C)(C)O3)cc2)C1 ; "Xphos Pd G2, K2CO3toluene/H2O, 80° C." ; CC(C)(C)OC(=O)N1CCCO[C@H](C(=O)N[C@H](C#N)Cc2ccc(-c3cc4n(c3)CCCNC4=O)cc2)C1]

To a mixture of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1.0 equiv) and 8-bromo-2H,3H,4H,5H-pyrrolo[1,2-a][1,4]diazepin-1-one (66 mg, 0.28 mmol, 1.2 equiv) in toluene (5 mL), H₂O (0.5 mL), were added K₂CO₃ (66 mg, 0.48 mmol, 2.0 equiv), 2nd Generation XPhos Pd catalyst (19 mg, 0.02 mmol, 0.1 equiv). The mixture was stirred at 80° C. for 3 h under nitrogen atmosphere. The reaction was cooled to room temperature, concentrated to remove the solvent, the residue was purified by silica gel column chromatography, eluted with PE/THF (1:1) to afford tert-butyl (2S)-2-{[(1S)-1-cyano-2-(4-{1-oxo-2H,3H,4H,5H-pyrrolo[1,2-a][1,4]diazepin-8-yl}phenyl)ethyl]carbamoyl}-1,4-oxazepane-4-carboxylate (80 mg, 63.8%) as a light yellow oil. LCMS (ES, m/z): [M+H]⁺: 522.

### Step 5. Synthesis of (2S)—N-[(1S)-1-cyano-2-(4-{1-oxo-2H,3H,4H,5H-pyrrolo[1,2-a][1,4]diazepin-8-yl}phenyl)ethyl]-1,4-oxazepane-2-carboxamide

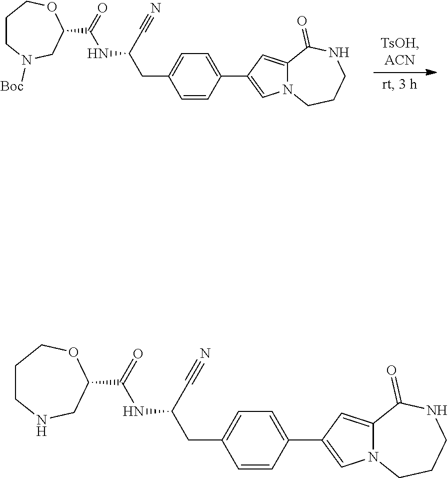 [SMILES: CC(C)(C)OC(=O)N1CCCO[C@H](C(=O)N[C@H](C#N)Cc2ccc(-c3cc4n(c3)CCCNC4=O)cc2)C1 ; N#C[C@H](Cc1ccc(-c2cc3n(c2)CCCNC3=O)cc1)NC(=O)[C@@H]1CNCCCO1]

Into a 8 mL vial were added tert-butyl (2S)-2-{[(1S)-1-cyano-2-(4-{1-oxo-2H,3H,4H,5H-pyrrolo[1,2-a][1,4]diazepin-8-yl}phenyl)ethyl]carbamoyl}-1,4-oxazepane-4-carboxylate (80 mg, 0.15 mmol, 1.0 equiv), ACN (3 mL) and TsOH·H₂O (87 mg, 0.45 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A12 (16.5 mg, 25.5%) as a white solid. LCMS (ES, m/z): [M+H]⁺: 422.1. ¹H NMR (400 MHz, DMSO-d₆) δ 8.57 (d, J=8.6 Hz, 1H), 7.70 (t, J=5.2 Hz, 1H), 7.53-7.45 (m, 2H), 7.41 (d, J=2.0 Hz, 1H), 7.24 (d, J=8.1 Hz, 2H), 6.96 (d, J=2.0 Hz, 1H), 4.97 (q, J=8.2 Hz, 1H), 4.18 (t, J=6.6 Hz, 2H), 3.98 (dd, J=7.9, 3.7 Hz, 1H), 3.93-3.81 (m, 1H), 3.76-3.69 (m, 1H), 3.17-3.00 (m, 4H), 3.03 (dd, J=14.2, 3.7 Hz, 1H), 2.82-2.71 (m, 1H), 2.65-2.54 (m, 2H), 2.07-1.99 (m, 2H), 1.83-1.60 (m, 2H).

### Example 12: Synthesis of (2S)—N-[(1S)-1-cyano-2-(4-{2-hydroxy-[1,3]oxazolo[4,5-b]pyridin-7-yl}phenyl)ethyl]-1,4-oxazepane-2-carboxamide (Compound A13)

### Step 1. Synthesis of 2-amino-4-bromopyridin-3-ol

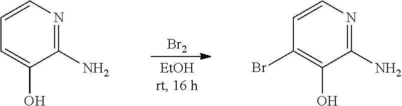 [SMILES: Nc1ncccc1O ; "Br2" ; Nc1nccc(Br)c1O]

To a solution of 2-amino-3-hydroxypyridine (1.0 g, 9.08 mmol) was dissolved in 5 ml of ethanol, the temperature was cooled to 0 C, liquid bromine (1.6 g, 9.98 mmol) was added slowly dropwise. After the addition, the temperature was raised to room temperature and the mixture was stirred overnight. The reaction solution was concentrated low temperature under vacuum. The residue was purified by silica gel column chromatography, eluted with MeOH in DCM (0%˜8%) to afford 2-amino-4-bromopyridin-3-ol (1.5 g, 43.69%) as a Brown yellow solid. LCMS (ES, m/z): [M+H]⁺: 189.

### Step 2. Synthesis of 7-bromo-[1,3]oxazolo[4,5-b]pyridin-2-ol

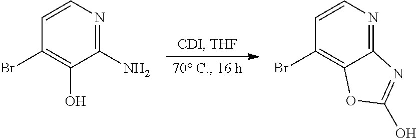

To a solution of 2-amino-4-bromopyridin-3-ol (1.1 g, 2.91 mmol, 1.00 equiv, 50%) in DMF (5.5 mL), was added CDI (0.94 g, 5.82 mmol, 2.00 equiv) and triethylamine (0.59 g, 5.820 mmol, 2.0 equiv) stirred for 4 h at 60° C. The mixture was allowed to cool down to room temperature. The residue was purified by reversed-phase flash chromatography with the following conditions: column, C18 silica gel; mobile phase, H₂O (0.05% FA) in MeCN, 10% to 40% gradient in 30 min; detector, UV 220 nm to afford 7-bromo-[1,3]oxazolo[4,5-b]pyridin-2-ol (200 mg, 31.97%) as a brown solid. LCMS (ES, m/z): [M+H]⁺: 215.

### Step 3. Synthesis of tert-butyl (2S)-2-{[(1S)-1-cyano-2-(4-{2-hydroxy-[1,3]oxazolo[4,5-b]pyridin-7-yl}phenyl)ethyl]carbamoyl}-1,4-oxazepane-4-carboxylate

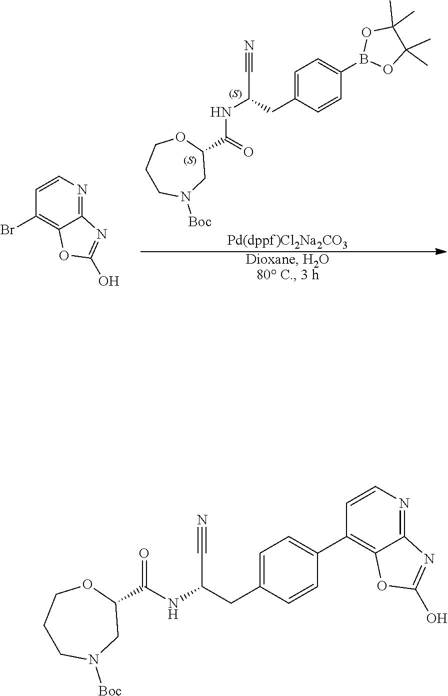

A solution of 7-bromo-[1,3]oxazolo[4,5-b]pyridin-2-ol (100 mg, 0.46 mmol, 1.00 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (278 mg, 0.55 mmol, 1.20 equiv), Na₂CO₃ (98 mg, 0.93 mmol, 2.00 equiv) and Pd(dppf)Cl₂ (34 mg, 0.04 mmol, 0.10 equiv) in dioxane (2 mL), H₂O (0.2 mL) was stirred for 2 h at 80° C. under nitrogen atmosphere. The reaction was concentrated and purified by silica gel column chromatography, eluted with PE/THF (4:1) to afford tert-butyl (2S)-2-{[(1S)-1-cyano-2-(4-{2-hydroxy-[1,3]oxazolo[4,5-b]pyridin-7-yl}phenyl)ethyl]carbamoyl}-1,4-oxazepane-4-carboxylate (52 mg, 22.03%) as a yellow semi-solid. LCMS (ES, m/z): [M+H]⁺: 508.

### Step 4. Synthesis of (2S)—N-[(1S)-1-cyano-2-(4-{2-hydroxy-[1,3]oxazolo[4,5-b]pyridin-7-yl}phenyl)ethyl]-1,4-oxazepane-2-carboxamide

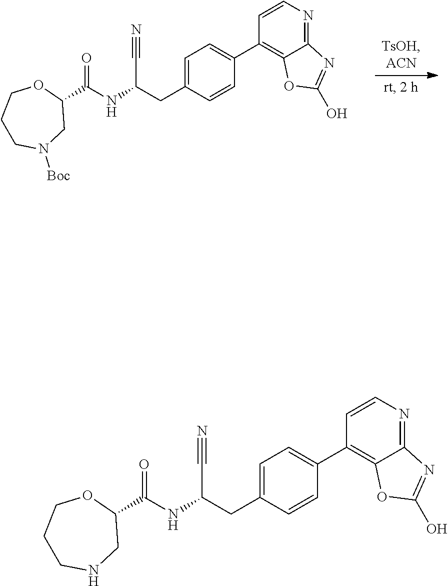 [SMILES: CC(C)(C)OC(=O)N1CCCO[C@H](C(=O)N[C@H](C#N)Cc2ccc(-c3ccnc4nc(O)oc34)cc2)C1 ; N#C[C@H](Cc1ccc(-c2ccnc3nc(O)oc23)cc1)NC(=O)[C@@H]1CNCCCO1]

Into a 8 mL vial were added tert-butyl (2S)-2-{[(1S)-1-cyano-2-(4-{2-hydroxy-[1,3]oxazolo[4,5-b]pyridin-7-yl}phenyl)ethyl]carbamoyl}-1,4-oxazepane-4-carboxylate (52 mg, 0.10 mmol, 1.00 equiv), TsOH (52.93 mg, 0.307 mmol, 3.00 equiv) and ACN (1 mL) at room temperature. The resulting mixture was stirred for additional 2 h at room temperature. The reaction mixture was purified by reversed-phase flash chromatography with the following conditions: column, C18 silica gel; mobile phase, MeCN in Water (0.1% NH3·H2O), 10% to 80% gradient in 10 min; detector, UV 254 nm. This resulted in Compound A13 (16 mg, 38.33%) as an off-white solid. LCMS (ES, m/z): [M+H]⁺: 408.2. ¹H NMR (400 MHz, DMSO-d6) δ 8.68 (d, J=8.6 Hz, 1H), 7.96 (d, J=5.6 Hz, 1H), 7.85 (d, J=8.2 Hz, 2H), 7.46 (d, J=8.0 Hz, 2H), 7.19 (d, J=5.7 Hz, 1H), 5.06 (q, J=8.2 Hz, 1H), 4.05 (dd, J=8.3, 3.6 Hz, 1H), 3.85 (dt, J=11.0, 5.2 Hz, 1H), 3.74 (td, J=7.7, 3.7 Hz, 1H), 3.19-3.16 (m, 2H), 3.07 (dd, J=14.3, 3.7 Hz, 1H), 2.85-2.81 (m, 1H), 2.73-2.63 (m, 1H), 2.57 (dd, J=14.2, 8.3 Hz, 1H), 1.76-1.73 (m, 2H).

### Example 13: Synthesis of (S)—N—((S)-1-cyano-2-(4-(4-hydroxypyrrolo[1,2-b]pyridazin-6-yl)phenyl)ethyl)-1,4-oxazepane-2-carboxamide (Compound A15)

### Step 1. Synthesis of (E)-1-(4-bromo-1H-pyrrol-2-yl)-3-(dimethylamino)prop-2-en-1-one

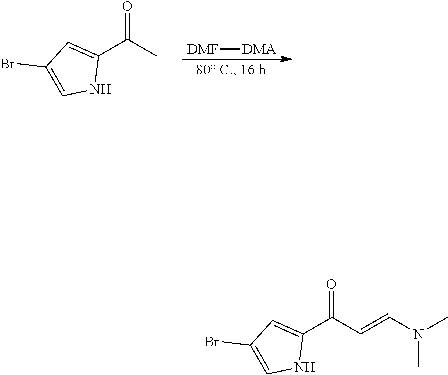

A solution of 1-(4-bromo-1H-pyrrol-2-yl)ethanone (2 g, 10.64 mmol, 1.0 equiv) in DMF-DMA (20 mL) was stirred at 80° C. for 16 h. The reaction was cooled to room temperature, quenched by the addition of water (30 mL). The aqueous layer was extracted with EtOAc (30 mL×2). The combined organic phase was washed with brine (20 mL×2), dried over anhydrous sodium sulfate. After filtration, the filtrate was concentrated, the resulting mixture was concentrated under vacuum. The residue was purified by silica gel column chromatography, eluted with PE/EA (5:1) to afford (2E)-1-(4-bromo-1H-pyrrol-2-yl)-3-(dimethylamino)prop-2-en-1-one (1 g, 38%) as a yellow oil. LCMS (ES) [M+1]⁺ m/z: 243.

### Step 2. Synthesis of 6-bromopyrrolo[1,2-b]pyridazin-4-ol

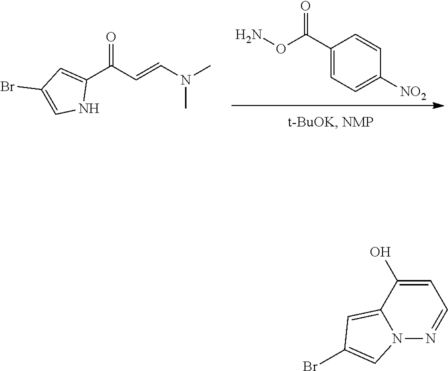

A solution of (2E)-1-(4-bromo-1H-pyrrol-2-yl)-3-(dimethylamino)prop-2-en-1-one (1 g, 4.11 mmol, 1.0 equiv) in NMP (30 mL) was treated with t-BuOK (0.69 g, 6.17 mmol, 1.5 equiv) at room temperature and stirred for 30 min. This was followed by the addition of amino 4-nitrobenzoate (1.12 g, 6.17 mmol, 1.5 equiv) at 0° C. The resulting mixture was stirred at 0° C. for 1 h, warmed to room temperature and stirred for additional 12 h. The mixture was acidified to pH 5 with HCl (aq.). The aqueous layer was extracted with EtOAc (20 mL×2). The residue was purified by silica gel column chromatography, eluted with PE/EA (2:1) to afford 6-bromopyrrolo[1,2-b]pyridazin-4-ol (400 mg, 46%) as a yellow solid. LCMS (ES) [M+1]⁺ m/z: 213.

### Step 3. Synthesis of tert-butyl (S)-2-(((S)-1-cyano-2-(4-(4-hydroxypyrrolo[1,2-b]pyridazin-6-yl)phenyl)ethyl)carbamoyl)-1,4-oxazepane-4-carboxylate

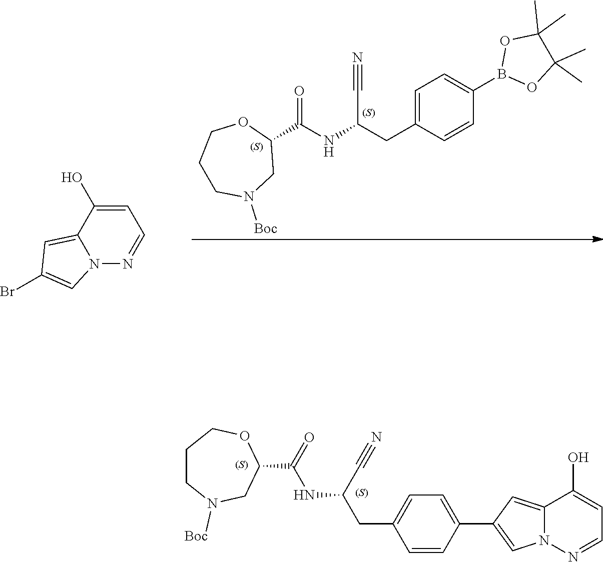

Into a 20 mL vial were added 6-bromopyrrolo[1,2-b]pyridazin-4-ol (400 mg, 1.88 mmol, 1.0 equiv), tert-butyl (S)-2-(((S)-1-cyano-2-(4-(4,4,5,5-tetramethyl-1,3,2-dioxaborolan-2-yl)phenyl)ethyl)carbamoyl)-1,4-oxazepane-4-carboxylate (938 mg, 1.88 mmol, 1.0 equiv), K₂CO₃ (519 mg, 3.76 mmol, 2.0 equiv), 4-{di-tert-butyl[dichloro({di-tert-butyl[4-(dimethylamino)phenyl]-lambda5-phosphanyl})palladio]-lambda5-phosphanyl}-N,N-dimethylaniline (133 mg, 0.19 mmol, 0.1 equiv) and dioxane (4 mL), H₂O (0.5 mL) at room temperature. The resulting mixture was stirred at 80° C. for 3 h under nitrogen atmosphere. The resulting mixture was concentrated under vacuum. The residue was purified by silica gel column chromatography, eluted with PE/THF (1:1) to afford tert-butyl (2S)-2-{[(1S)-1-cyano-2-(4-{4-hydroxypyrrolo[1,2-b]pyridazin-6-yl}phenyl)ethyl]carbamoyl}-1,4-oxazepane-4-carboxylate (180 mg, 19%) as a off-white solid. LCMS (ES) [M+1]⁺ m/z: 506.

### Step 4. Synthesis of (S)—N—((S)-1-cyano-2-(4-(4-hydroxypyrrolo[1,2-b]pyridazin-6-yl)phenyl)ethyl)-1,4-oxazepane-2-carboxamide

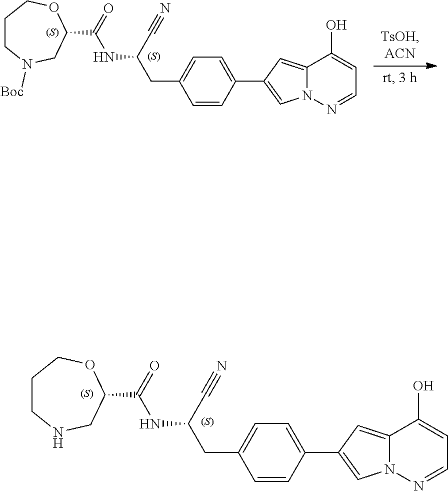

Into a 8 mL vial were added tert-butyl (2S)-2-{[(1S)-1-cyano-2-(4-{4-hydroxypyrrolo[1,2-b]pyridazin-6-yl}phenyl)ethyl]carbamoyl}-1,4-oxazepane-4-carboxylate (150 mg, 0.30 mmol, 1.0 equiv), acetonitrile (3 mL) and TsOH (265 mg, 0.89 mmol, 3.0 equiv) at room temperature. The resulting mixture was stirred at room temperature for 3 h. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A15 (19.5 mg, 16%) as off-white solid. LCMS (ES) [M+1]⁺ m/z: 406.4. ¹H NMR (300 MHz, DMSO-d₆) δ 8.61 (d, J=8.5 Hz, 1H), 8.17 (d, J=1.9 Hz, 1H), 7.87 (d, J=5.4 Hz, 1H), 7.70 (d, J=8.0 Hz, 2H), 7.30 (d, J=8.0 Hz, 2H), 6.91 (d, J=1.9 Hz, 1H), 5.96 (d, J=5.4 Hz, 1H), 5.00 (q, J=8.1 Hz, 1H), 4.01 (dd, J=7.9, 3.6 Hz, 1H), 3.90-3.82 (m, 1H), 3.77-3.68 (m, 1H), 3.19-3.13 (m, 2H), 3.09-3.02 (m, 1H), 2.83-2.72 (m, 1H), 2.70-2.58 (m, 2H), 1.91-1.67 (m, 2H).

### Example 14: Synthesis of (2S)—N-[(1S)-1-cyano-2-(4-{5H-pyrrolo[2,3-b]pyrazin-2-yl}phenyl)ethyl]-1,4-oxazepane-2-carboxamide (Compound A16)

### Step 1. Synthesis of tert-butyl (2S)-2-{[(1S)-1-cyano-2-(4-{5H-pyrrolo[2,3-b]pyrazin-2-yl}phenyl)ethyl]carbamoyl}-1,4-oxazepane-4-carboxylate

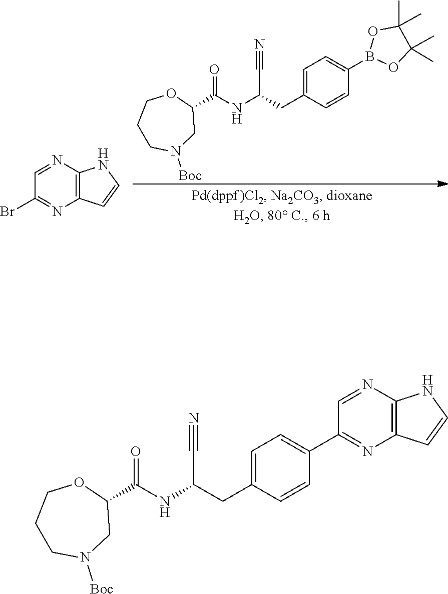 [SMILES: Brc1cnc2[nH]ccc2n1 ; CC(C)(C)OC(=O)N1CCCO[C@H](C(=O)N[C@H](C#N)Cc2ccc(B3OC(C)(C)C(C)(C)O3)cc2)C1 ; CC(C)(C)OC(=O)N1CCCO[C@H](C(=O)N[C@H](C#N)Cc2ccc(-c3cnc4[nH]ccc4n3)cc2)C1]

To a stirred solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (300 mg, 0.601 mmol, 1.0 equiv) and 2-bromo-5H-pyrrolo[2,3-b]pyrazine (130 mg, 0.661 mmol, 1.1 equiv) in dioxane (6 mL) H₂O (0.6 mL) were added Na₂CO₃ (127 mg, 1.202 mmol, 2.0 equiv) and Pd(dppf)Cl₂ (43 mg, 0.060 mmol, 0.1 equiv). The resulting mixture was stirred at 80° C. for 6 h under nitrogen atmosphere. The mixture was allowed to cool down to room temperature. The resulting mixture was concentrated under reduced pressure. The residue was purified by silica gel column chromatography, eluted with PE/THF (1:1) to afford tert-butyl (2S)-2-{[(1S)-1-cyano-2-(4-{5H-pyrrolo[2,3-b]pyrazin-2-yl}phenyl)ethyl]carbamoyl}-1,4-oxazepane-4-carboxylate (150 mg, 50.9%) as an off-white solid. LCMS (ES) [M+1]⁺ m/z: 491.

### Step 2. Synthesis of (2S)—N-[(1S)-1-cyano-2-(4-{5H-pyrrolo[2,3-b]pyrazin-2-yl}phenyl)ethyl]-1,4-oxazepane-2-carboxamide

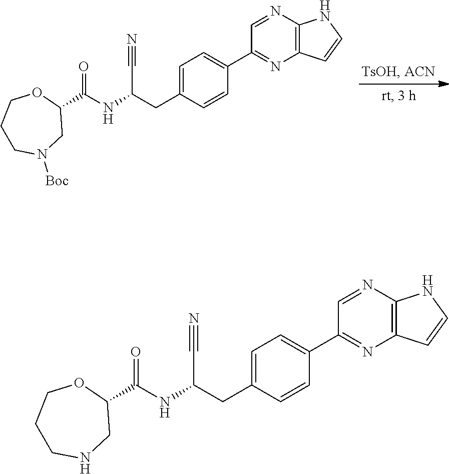 [SMILES: CC(C)(C)OC(=O)N1CCCO[C@H](C(=O)N[C@H](C#N)Cc2ccc(-c3cnc4[nH]ccc4n3)cc2)C1 ; N#C[C@H](Cc1ccc(-c2cnc3[nH]ccc3n2)cc1)NC(=O)[C@@H]1CNCCCO1]

To a stirred solution of tert-butyl (2S)-2-{[(1S)-1-cyano-2-(4-{5H-pyrrolo[2,3-b]pyrazin-2-yl}phenyl)ethyl]carbamoyl}-1,4-oxazepane-4-carboxylate (100 mg, 0.204 mmol, 1.0 equiv) in ACN (2 mL) was added TsOH (105 mg, 0.612 mmol, 3.0 equiv). The resulting mixture was stirred at room temperature for 3 h. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound A16 (30 mg, 37.6%) as a white solid. LCMS (ES) [M+1]⁺ m/z: 391. ¹H NMR (300 MHz, DMSO-d6) δ 12.08 (s, 1H), 8.84 (s, 1H), 8.63 (d, J=8.6 Hz, 1H), 8.13-8.04 (m, 2H), 7.90 (d, J=3.6 Hz, 1H), 7.43 (d, J=8.2 Hz, 2H), 6.68 (d, J=3.5 Hz, 1H), 5.12-4.98 (m, 1H), 4.00 (dd, J=7.9, 3.7 Hz, 1H), 3.93-3.80 (m, 1H), 3.73 (ddd, J=12.1, 7.6, 4.2 Hz, 1H), 3.28-3.14 (m, 2H), 3.03 (dd, J=14.3, 3.7 Hz, 1H), 2.76 (dt, J=11.6, 5.3 Hz, 1H), 2.68-2.52 (m, 2H), 2.02-1.51 (m, 2H).

### Example 15: Synthesis of (2S)—N-[(1S)-1-cyano-2-(4-{2-oxo-1H,3H-imidazo[4,5-c]pyridin-7-yl}phenyl)ethyl]-1,4-oxazepane-2-carboxamide (Compound A17)

### Step 1. Synthesis of 7-bromo-1H,3H-imidazo[4,5-c]pyridin-2-one

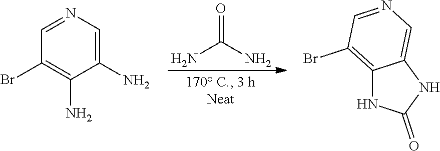

To a stirred mixture of pyridine, 3-bromo-4,5-diamino- (1.5 g, 7.97 mmol, 1.0 equiv) and urea (1.44 g, 23.93 mmol, 3.0 equiv) in a sealed tube. The resulting mixture was stirred for 3 h at 170° C. The resulting mixture was cooled to room temperature and washed with H₂O (10 mL), re-crystallized from EtOAc (8 mL) to afford 7-bromo-1H,3H-imidazo[4,5-c]pyridin-2-one (1.2 g, 70.2%) as a light yellow solid. LCMS (ES) [M+1]⁺ m/z: 214.

### Step 2. Synthesis of tert-butyl (2S)-2-{[(1S)-1-cyano-2-(4-{2-oxo-1H,3H-imidazo[4,5-c]pyridin-7-yl}phenyl)ethyl]carbamoyl}-1,4-oxazepane-4-carboxylate

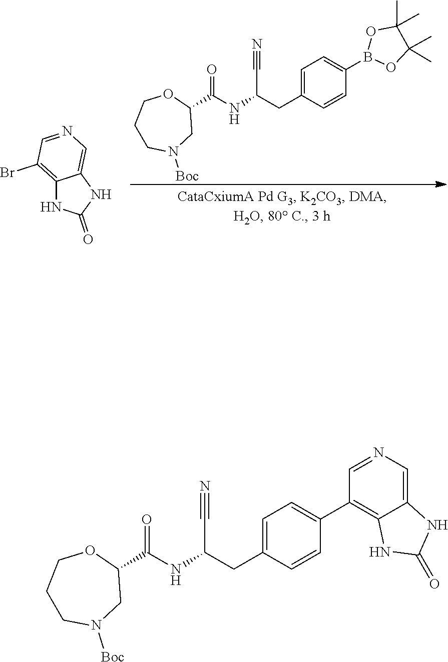 [SMILES: O=c1[nH]c2cncc(Br)c2[nH]1 ; CC(C)(C)OC(=O)N1CCCO[C@H](C(=O)N[C@H](C#N)Cc2ccc(B3OC(C)(C)C(C)(C)O3)cc2)C1 ; CC(C)(C)OC(=O)N1CCCO[C@H](C(=O)N[C@H](C#N)Cc2ccc(-c3cncc4[nH]c(=O)[nH]c34)cc2)C1]

To a stirred solution of 7-bromo-1H,3H-imidazo[4,5-c]pyridin-2-one (111 mg, 0.520 mmol, 1.3 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (200 mg, 0.400 mmol, 1.0 equiv) in DMA (3 mL) and H₂O (0.6 mL) were added cataCXium-A-Pd-G3 (58 mg, 0.080 mmol, 0.2 equiv) and K₂CO₃ (166 mg, 1.200 mmol, 3.0 equiv). The resulting mixture was stirred for 3 h at 80° C. under nitrogen atmosphere. The mixture was allowed to cool down to room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in tert-butyl (2S)-2-{[(1S)-1-cyano-2-(4-{2-oxo-1H,3H-imidazo[4,5-c]pyridin-7-yl}phenyl)ethyl]carbamoyl}-1,4-oxazepane-4-carboxylate (80 mg, 39.4%) as a white solid. LCMS (ES) [M+1]⁺ m/z: 507.

### Step 3. Synthesis of (2S)—N-[(1S)-1-cyano-2-(4-{2-oxo-1H,3H-imidazo[4,5-c]pyridin-7-yl}phenyl)ethyl]-1,4-oxazepane-2-carboxamide

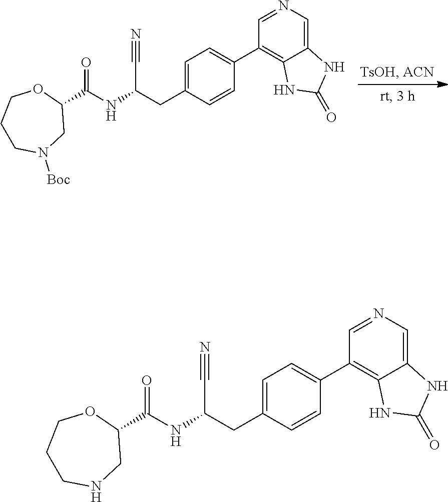 [SMILES: CC(C)(C)OC(=O)N1CCCO[C@H](C(=O)N[C@H](C#N)Cc2ccc(-c3cncc4[nH]c(=O)[nH]c34)cc2)C1 ; N#C[C@H](Cc1ccc(-c2cncc3[nH]c(=O)[nH]c23)cc1)NC(=O)[C@@H]1CNCCCO1]

To a stirred solution of tert-butyl (2S)-2-{[(1S)-1-cyano-2-(4-{2-oxo-1H,3H-imidazo[4,5-c]pyridin-7-yl}phenyl)ethyl]carbamoyl}-1,4-oxazepane-4-carboxylate (100 mg, 0.197 mmol, 1.0 equiv) in ACN (2 mL) was added TsOH (81 mg, 0.474 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound A17 (20 mg, 31.1%) as a white solid. LCMS (ES) [M+1]⁺ m/z: 407. ¹H NMR (300 MHz, DMSO-d6) δ 8.64 (dd, J=8.5, 3.3 Hz, 1H), 8.22-8.11 (m, 2H), 7.56 (dd, J=8.0, 5.9 Hz, 2H), 7.47-7.38 (m, 2H), 5.15-4.92 (m, 1H), 4.06-3.80 (m, 2H), 3.72 (ddd, J=12.1, 7.6, 4.0 Hz, 1H), 3.29-3.12 (m, 2H), 3.17-2.97 (m, 1H), 2.88-2.61 (m, 2H), 2.51-2.41 (m, 1H), 1.82-1.66 (m, 2H).

### Example 16: Synthesis of (2S)—N-[(1S)-1-cyano-2-[4-(5-methyl-6-oxo-1,5-naphthyridin-3-yl)phenyl]ethyl]-1,4-oxazepane-2-carboxamide (Compound A18)

### Step 1. Synthesis of 7-bromo-1,5-naphthyridin-1-ium-1-olate

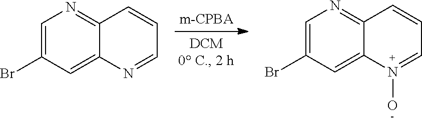

To a stirred solution of 3-bromo-1,5-naphthyridine (1.0 g, 4.78 mmol, 1.0 equiv) in DCM (20 mL) were added m-CPBA (0.99 g, 5.74 mmol, 1.2 equiv) in portions at 0° C. The resulting mixture was stirred for additional 2 h at room temperature. The resulting mixture was diluted with DCM (20 mL). The residue was washed with sat. Na₂SO₃ (aq.) (2×30 mL), sat, NaHCO₃ (aq.) (2×30 mL), brine (2×30 mL), dried over anhydrous Na₂SO₄. After filtration, the filtrate was concentrated under reduced pressure. The residue was purified by silica gel column chromatography, eluted with PE/THF (2:3) to afford 7-bromo-1,5-naphthyridin-1-ium-1-olate (0.7 g, 65.02%) as a white solid. LCMS (ES, m/z): [M+H]⁺: 225. ¹H NMR (300 MHz, Chloroform-d) δ 9.26 (s, 1H), 9.07 (s, 1H), 8.60 (s, 1H), 8.03 (d, J=8.6 Hz, 1H), 7.61 (d, J=8.3 Hz, 1H).

### Step 2. Synthesis of 7-bromo-1H-1,5-naphthyridin-2-one

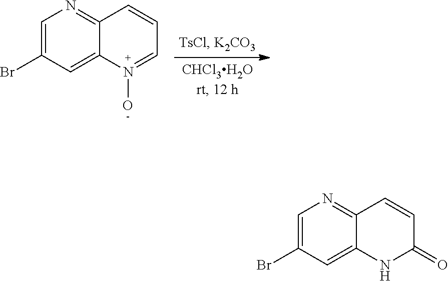

To a stirred mixture of 7-bromo-1,5-naphthyridin-1-ium-1-olate (0.7 g, 3.11 mmol, 1.0 equiv) and K₂CO₃ (1.46 g, 10.57 mmol, 3.4 equiv) in CHCl₃ (14 mL) and H₂O (4 mL) were added TsCl (0.71 g, 3.73 mmol, 1.2 equiv) in portions at room temperature. The resulting mixture was stirred for additional overnight at room temperature. The resulting mixture was diluted with water (20 mL). The precipitated solids were collected by filtration and washed with water (3×10 mL). The resulting solid was dried under infrared light. This resulted in 7-bromo-1H-1,5-naphthyridin-2-one (0.6 g, 85.71%) as a light yellow solid. LCMS (ES, m/z): [M+H]⁺: 225. ¹H NMR (300 MHz, DMSO-d6) δ 11.97 (s, 1H), 8.56 (d, J=2.0 Hz, 1H), 7.93 (d, J=9.8 Hz, 1H), 7.86 (d, J=2.1 Hz, 1H), 6.79 (d, J=9.8 Hz, 1H).

### Step 3. Synthesis of 7-bromo-1-methyl-1,5-naphthyridin-2-one

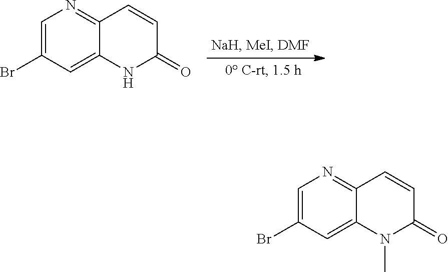

To a stirred solution of 7-bromo-1H-1,5-naphthyridin-2-one (400 mg, 1.78 mmol, 1.0 equiv) in DMF (10 mL) were added NaH (85.31 mg, 3.55 mmol, 2.0 equiv) in portions at 0° C. The resulting mixture was stirred for additional 30 min at 0° C. To the above mixture was added CH₃I (302.74 mg, 2.13 mmol, 1.2 equiv) dropwise at 0° C. The resulting mixture was stirred for additional 1 h at room temperature. The reaction was quenched by the addition of water/ice (10 mL) at 0° C. The resulting mixture was diluted with water (20 mL). The resulting mixture was extracted with EtOAc (3×15 mL). The combined organic layers were washed with water (2×20 mL) and brine (2×20 mL), dried over anhydrous Na₂SO₄. After filtration, the filtrate was concentrated under reduced pressure. This resulted in 7-bromo-1-methyl-1,5-naphthyridin-2-one (300 mg, 70.60%) as a light yellow solid. LCMS (ES, m/z): [M+H]⁺: 239. ¹H NMR (300 MHz, DMSO-d6) δ 8.62 (d, J=1.8 Hz, 1H), 8.28 (d, J=2.0 Hz, 1H), 7.91 (d, J=9.7 Hz, 1H), 6.90 (d, J=9.8 Hz, 1H), 3.60 (s, 3H).

### Step 4. Synthesis of tert-butyl (2S)-2-{[(1S)-1-cyano-2-[4-(5-methyl-6-oxo-1,5-naphthyridin-3-yl)phenyl]ethyl]carbamoyl}-1,4-oxazepane-4-carboxylate

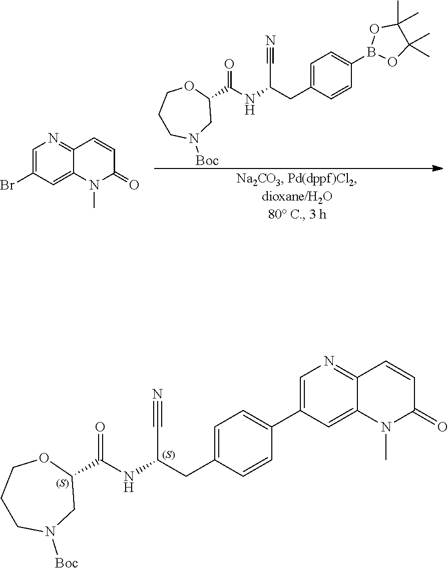

To a stirred mixture of 7-bromo-1-methyl-1,5-naphthyridin-2-one (80 mg, 0.34 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (217.26 mg, 0.44 mmol, 1.3 equiv) in dioxane (4 mL) and H₂O (0.4 mL) were added Na₂CO₃ (70.93 mg, 0.67 mmol, 2.0 equiv) and Pd(dppf)Cl₂·CH₂Cl₂ (27.26 mg, 0.03 mmol, 0.1 equiv) in portion at room temperature under nitrogen atmosphere. The resulting mixture was stirred for additional 3 h at 80° C. The resulting mixture was filtered, the filter cake was washed with EtOAc (3×10 mL). The filtrate was concentrated under reduced pressure. The residue was purified by silica gel column chromatography, eluted with PE/THF (2:3) to afford tert-butyl (2S)-2-{[(1S)-1-cyano-2-[4-(5-methyl-6-oxo-1,5-naphthyridin-3-yl)phenyl]ethyl]carbamoyl}-1,4-oxazepane-4-carboxylate (115 mg, 64.65%) as a white semi-solid. LCMS (ES, m/z): [M+H]⁺: 532.

### Step 5. Synthesis of (2S)—N-[(1S)-1-cyano-2-[4-(5-methyl-6-oxo-1,5-naphthyridin-3-yl)phenyl]ethyl]-1,4-oxazepane-2-carboxamide

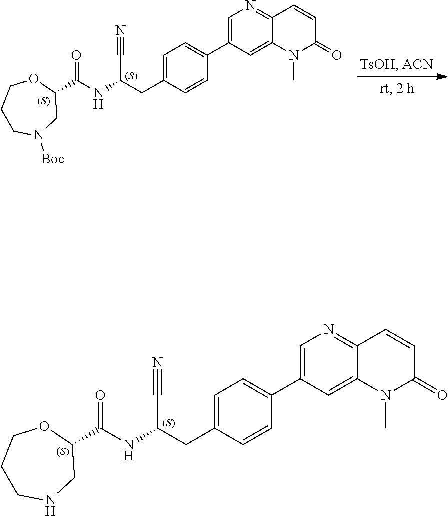

To a stirred solution of tert-butyl (2S)-2-{[(1S)-1-cyano-2-[4-(5-methyl-6-oxo-1,5-naphthyridin-3-yl)phenyl]ethyl]carbamoyl}-1,4-oxazepane-4-carboxylate (110 mg, 0.21 mmol, 1.0 equiv) in ACN (3 mL) were added TsOH·H₂O (118.07 mg, 0.62 mmol, 3.0 equiv) in portions at room temperature. The resulting mixture was stirred for additional 2 h at room temperature. The crude product was purified by Prep-HPLC with the following conditions (column, C18 silica gel; mobile phase, MeCN in Water (0.1% NH₃·H₂O), 20% to 40% gradient in 10 min; detector, UV 254 nm.) to afford Compound A18 (26 mg, 29.12%) as a white solid. LCMS (ES, m/z): [M+H]⁺: 432.4. ¹H NMR (300 MHz, DMSO-d₆) δ 8.86 (d, J=1.8 Hz, 1H), 8.64 (d, J=8.5 Hz, 1H), 8.14 (d, J=1.9 Hz, 1H), 7.97 (d, J=9.7 Hz, 1H), 7.90 (dd, J=8.4, 2.1 Hz, 2H), 7.49 (d, J=7.9 Hz, 2H), 6.88 (d, J=9.7 Hz, 1H), 5.07 (q, J=8.2 Hz, 1H), 4.00 (dd, J=7.9, 3.6 Hz, 1H), 3.90-3.81 (m, 1H), 3.77-3.68 (m, 4H), 3.65-3.25 (m, 1H), 3.28-3.21 (m, 2H), 3.03 (dd, J=14.3, 3.7 Hz, 1H), 2.81-2.71 (m, 1H), 2.66-2.55 (m, 2H), 1.84-1.63 (m, 2H).

### Example 17: Synthesis of (2S)—N-[(1S)-2-(4-{2-carbamoyl-3-methylthieno[2,3-b]pyridin-5-yl}phenyl)-1-cyanoethyl]-1,4-oxazepane-2-carboxamide (Compound A19)

### Step 1. Synthesis of methyl 5-bromo-3-methylthieno[2,3-b]pyridine-2-carboxylate

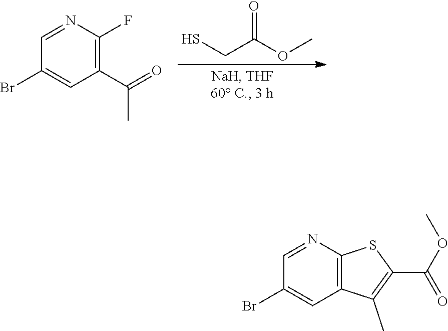

To a stirred solution of 1-(5-bromo-2-fluoropyridin-3-yl)ethanone (3.0 g, 13.76 mmol, 1.0 equiv) and methyl thioglycolate (1.8 g, 16.51 mmol, 1.2 equiv) in THF (30 mL) was added NaH (1.1 g, 27.52 mmol, 2.0 equiv, 60% in mineral oil) in portions at 0° C. under nitrogen atmosphere. The resulting mixture was stirred for 5 min at room temperature. The resulting mixture was stirred for additional 3 h at 60° C. This resulted in methyl 5-bromo-3-methylthieno[2,3-b]pyridine-2-carboxylate (3.0 g, 76.2%) as yellow oil. The crude product mixture was used in the next step directly without further purification. LCMS (ES, m/z): [M+H]⁺: 286.

### Step 2. Synthesis of 5-bromo-3-methylthieno[2,3-b]pyridine-2-carboxylic acid

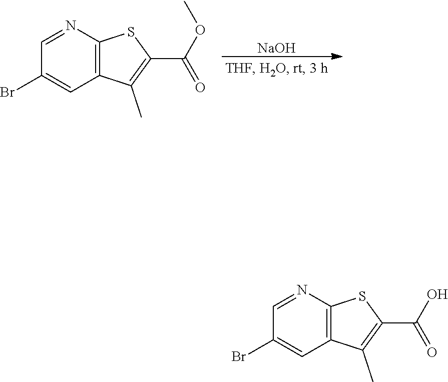

A solution of methyl 5-bromo-3-methylthieno[2,3-b]pyridine-2-carboxylate (3.0 g, 10.48 mmol, 1.0 equiv) and NaOH (0.8 g, 20.96 mmol, 2.0 equiv) in THF (30 mL) was stirred for 3 h at room temperature. The mixture was acidified to pH 4 with HCl (1 M). The resulting mixture was extracted with EtOAc (3×30 mL). The combined organic layer was washed with brine (3×30 mL), dried over anhydrous Na₂SO₄. After filtration, the filtrate was concentrated under reduced pressure. This resulted in 5-bromo-3-methylthieno[2,3-b]pyridine-2-carboxylic acid (3 g, crude) as yellow oil. LCMS (ES, m/z): [M+H]⁺: 272.

### Step 3. Synthesis of 5-bromo-3-methylthieno[2,3-b]pyridine-2-carboxamide

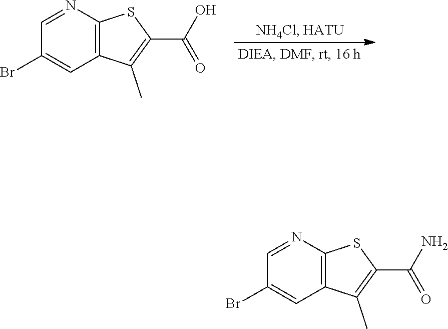

A solution of 5-bromo-3-methylthieno[2,3-b]pyridine-2-carboxylic acid (3.0 g, 11.02 mmol, 1.0 equiv) in DMF (30 mL) was treated with NH₄Cl (0.6 g, 11.02 mmol, 1.0 equiv) and DIEA (4.3 g, 33.07 mmol, 3.0 equiv). This was followed by the addition of HATU (5.0 g, 13.23 mmol, 1.2 equiv) in portions at 0° C. The resulting mixture was stirred for 16 h at room temperature. The reaction was quenched with water (40 mL), extracted with ethyl acetate (40 mL×2). The combined organic phase was washed with brine (30 mL×3), dried over anhydrous sodium sulfate. After filtered, the filtrate was concentrated under reduced pressure. The residue was purified by silica gel column chromatography, eluted with PE/THF (1:1) to afford 5-bromo-3-methylthieno[2,3-b]pyridine-2-carboxamide (100 mg, 78%) as white semi-solid. LCMS (ES, m/z): [M+H]⁺: 271.

### Step 4. Synthesis of (2S)-2-{[(1S)-2-(4-{2-carbamoyl-3-methylthieno[2,3-b]pyridin-5-yl}phenyl)-1-cyanoethyl]carbamoyl}-1,4-oxazepane-4-carboxylate

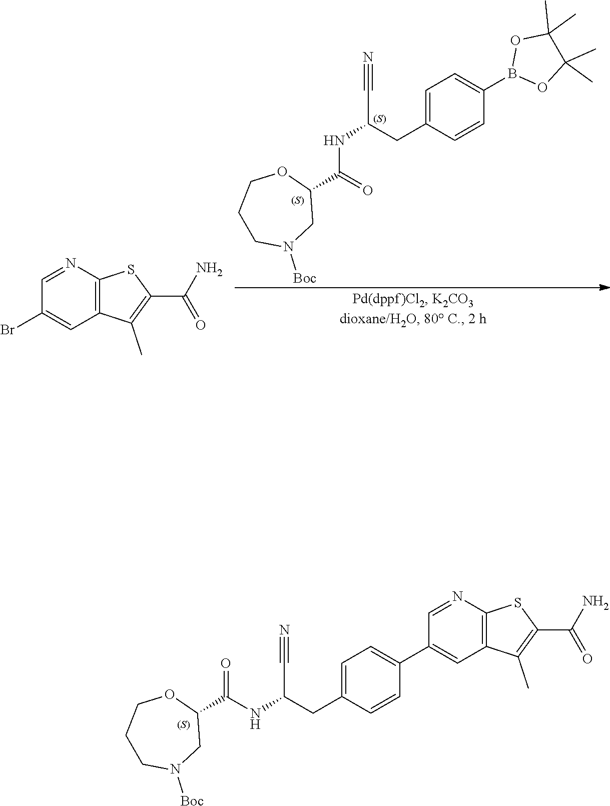

To a solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (170 mg, 0.34 mmol, 1.0 equiv) and 5-bromo-3-methylthieno[2,3-b]pyridine-2-carboxamide (184 mg, 0.68 mmol, 2.0 equiv) in 1,4-dioxane (5 mL) and H₂O (0.5 mL). This was followed by the addition of K₂CO₃ (94 mg, 0.68 mmol, 2.0 equiv), Pd(dppf)Cl₂ (24 mg, 0.03 mmol, 0.1 equiv) in sequence. The mixture was stirred for 2 h at 80° C. under nitrogen atmosphere. The residue was purified by silica gel column chromatography, eluted with PE/THF (1:1) to afford tert-butyl (2S)-2-{[(1S)-2-(4-{2-carbamoyl-3-methylthieno[2,3-b]pyridin-5-yl}phenyl)-1-cyanoethyl]carbamoyl}-1,4-oxazepane-4-carboxylate (120 mg, 62.5%) as white semi-solid. LCMS (ES, m/z): [M+H]⁺: 564.

### Step 5. Synthesis of (2S)—N-[(1S)-2-(4-{2-carbamoyl-3-methylthieno[2,3-b]pyridin-5-yl}phenyl)-1-cyanoethyl]-1,4-oxazepane-2-carboxamide

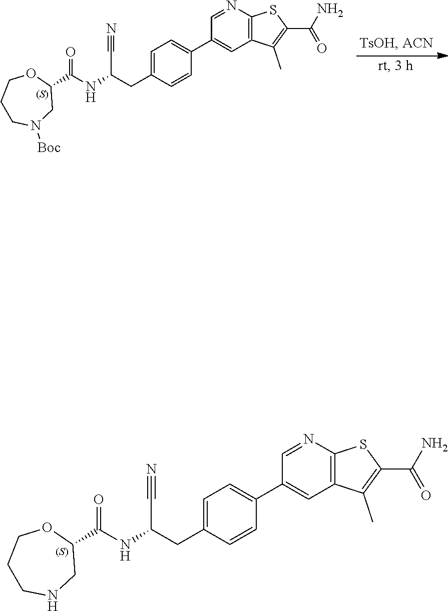

Into a 25 mL round-bottom flask were added tert-butyl (2S)-2-{[(1S)-2-(4-{2-carbamoyl-3-methylthieno[2,3-b]pyridin-5-yl}phenyl)-1-cyanoethyl]carbamoyl}-1,4-oxazepane-4-carboxylate (120 mg, 0.21 mmol, 1.0 equiv), ACN (3 mL) and TsOH·H₂O (121 mg, 0.63 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A19 (20.3 mg, 20.6%) as white solid. LCMS (ES, m/z): [M+H]⁺: 464.1. ¹H NMR (400 MHz, DMSO-d₆) δ 8.97 (d, J=2.3 Hz, 1H), 8.64 (d, J=8.5 Hz, 1H), 8.53 (d, J=2.2 Hz, 1H), 7.87-7.78 (m, 4H), 7.47 (d, J=8.2 Hz, 2H), 5.06 (q, J=8.2 Hz, 1H), 4.00 (dd, J=7.8, 3.7 Hz, 1H), 3.89-3.83 (m, 1H), 3.76-3.70 (m, 1H), 3.27-3.23 (m, 2H), 3.04 (dd, J=14.3, 3.7 Hz, 1H), 2.80-2.73 (m, 1H), 2.68 (s, 3H), 2.65-2.53 (m, 2H), 1.80-1.69 (m, 2H).

### Example 18: Synthesis of 7-{4-[(2S)-2-cyano-2-[(2S)-1,4-oxazepan-2-ylformamido]ethyl]phenyl}-3,4-dihydro-2H-quinoline-1-carboxamide (Compound A20)

### Step 1. Synthesis of 7-bromo-3,4-dihydro-2H-quinoline-1-carboxamide

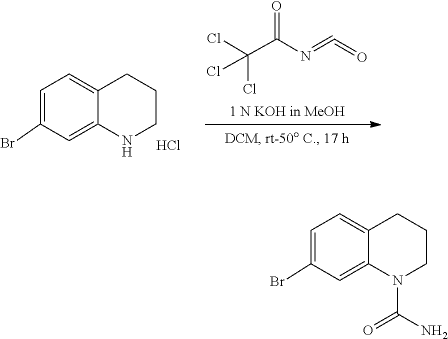 [SMILES: Brc1ccc2c(c1)NCCC2 ; O=C=NC(=O)C(Cl)(Cl)Cl ; Cl ; NC(=O)N1CCCc2ccc(Br)cc21]

To a stirred solution of 7-bromo-1,2,3,4-tetrahydroquinoline hydrochloride (400 mg, 1.609 mmol, 1.0 equiv) and DIEA (416 mg, 3.218 mmol, 2.0 equiv) in DCM (8 mL) were added trichloroethanecarbonyl isocyanate (454 mg, 2.413 mmol, 1.5 equiv). The resulting mixture was stirred for 1 h at room temperature. To the above mixture was added KOH (451 mg, 8.045 mmol, 5.0 equiv) in MeOH (8 mL). The resulting mixture was stirred for 16 h at 50° C. The mixture was allowed to cool down to room temperature. The resulting mixture was diluted with water (30 mL). The resulting mixture was extracted with CH₂Cl₂ (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/THF (2:1) to afford 7-bromo-3,4-dihydro-2H-quinoline-1-carboxamide (300 mg, 73.0%) as a white solid. LCMS (ES) [M+1]⁺ m/z: 255.

### Step 2. Synthesis of tert-butyl (2S)-2-{[(1S)-2-[4-(1-carbamoyl-3,4-dihydro-2H-quinolin-7-yl)phenyl]-1-cyanoethyl]carbamoyl}-1,4-oxazepane-4-carboxylate

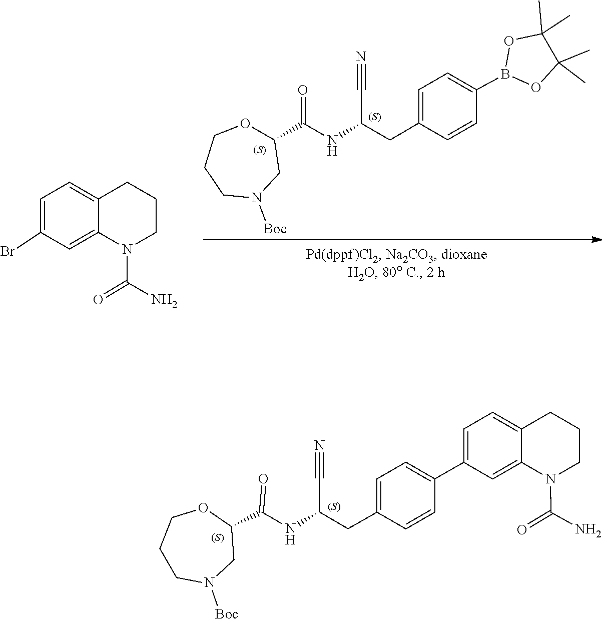

To a stirred solution of 7-bromo-3,4-dihydro-2H-quinoline-1-carboxamide (80 mg, 0.314 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (156 mg, 0.314 mmol, 1.0 equiv) in dioxane (2 mL) and H₂O (0.2 mL) were added Na₂CO₃ (66 mg, 0.628 mmol, 2.0 equiv) and Pd(dppf)Cl₂ (22 mg, 0.031 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (1:1) to afford tert-butyl (2S)-2-{[(1S)-2-[4-(1-carbamoyl-3,4-dihydro-2H-quinolin-7-yl)phenyl]-1-cyanoethyl]carbamoyl}-1,4-oxazepane-4-carboxylate (110 mg, 64.0%) as a light yellow oil. LCMS (ES) [M+1]⁺ m/z: 548.

### Step 3. Synthesis of 7-{4-[(2S)-2-cyano-2-[(2S)-1,4-oxazepan-2-ylformamido]ethyl]phenyl}-3,4-dihydro-2H-quinoline-1-carboxamide

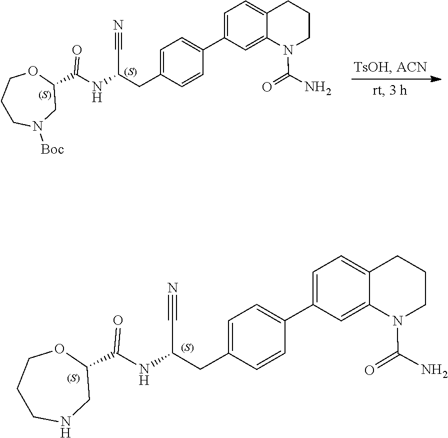

To a stirred solution of tert-butyl (2S)-2-{[(1S)-2-[4-(1-carbamoyl-3,4-dihydro-2H-quinolin-7-yl)phenyl]-1-cyanoethyl]carbamoyl}-1,4-oxazepane-4-carboxylate (110 mg, 0.201 mmol, 1.0 equiv) in ACN (2 mL) was added TsOH (103 mg, 0.603 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound A20 (22 mg, 24.4%) as a white solid. LCMS (ES) [M+1]⁺ m/z: 448. ¹H NMR (300 MHz, DMSO-d6) δ 8.60 (d, J=8.5 Hz, 1H), 7.79 (d, J=1.6 Hz, 1H), 7.53 (d, J=8.1 Hz, 2H), 7.35 (d, J=8.1 Hz, 2H), 7.25-7.12 (m, 2H), 6.33 (s, 2H), 5.01 (q, J=8.2 Hz, 1H), 3.99 (dd, J=7.9, 3.7 Hz, 1H), 3.91-3.78 (m, 1H), 3.72 (ddd, J=12.0, 7.4, 4.2 Hz, 1H), 3.60 (t, J=6.0 Hz, 2H), 3.24-3.14 (m, 2H), 3.02 (dd, J=14.3, 3.8 Hz, 1H), 2.83-2.67 (m, 3H), 2.67-2.51 (m, 2H), 1.86 (p, J=6.2 Hz, 2H), 1.80-1.65 (m, 2H).

### Example 19: Synthesis of (2S)—N-[(1S)-1-cyano-2-[4-(1-methyl-2-oxo-3,4-dihydroquinolin-6-yl)phenyl]ethyl]-1,4-oxazepane-2-carboxamide (Compound A21)

### Step 1. Synthesis of tert-butyl (2S)-2-{[(1S)-1-cyano-2-[4-(1-methyl-2-oxo-3,4-dihydroquinolin-6-yl)phenyl]ethyl]carbamoyl}-1,4-oxazepane-4-carboxylate

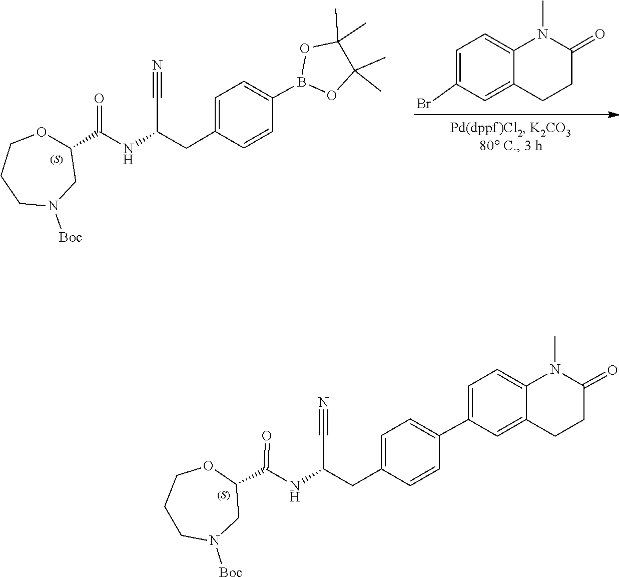

To a solution of 6-bromo-1-methyl-3,4-dihydroquinolin-2-one (60 mg, 0.25 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (162 mg, 0.32 mmol, 1.3 equiv), in 1,4-dioxane (5 mL) and H₂O (0.5 mL). Pd(dppf)Cl₂ (18 mg, 0.02 mmol, 0.1 equiv) and K₂CO₃ (69 mg, 0.50 mmol, 2.0 equiv) were added in sequence. The mixture was stirred for 2 h at 80° C. under nitrogen atmosphere. The reaction was cooled to room temperature, concentrated to remove the solvent, the residue was purified by silica gel column chromatography, eluted with PE/THF (1:1) to afford tert-butyl (2S)-2-{[(1S)-1-cyano-2-[4-(1-methyl-2-oxo-3,4-dihydroquinolin-6-yl)phenyl]ethyl]carbamoyl}-1,4-oxazepane-4-carboxylate (100 mg, 75%) as white foam solid. LCMS (ES, m/z): [M+H]⁺: 533.

### Step 2. Synthesis of (2S)—N-[(1S)-1-cyano-2-[4-(1-methyl-2-oxo-3,4-dihydroquinolin-6-yl)phenyl]ethyl]-1,4-oxazepane-2-carboxamide

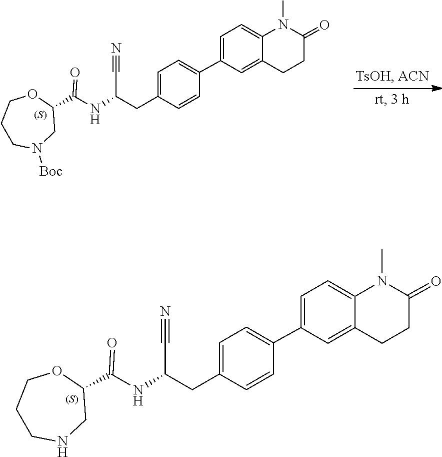

Into a 25 mL round-bottom flask were added tert-butyl (2S)-2-{[(1S)-1-cyano-2-[4-(1-methyl-2-oxo-3,4-dihydroquinolin-6-yl)phenyl]ethyl]carbamoyl}-1,4-oxazepane-4-carboxylate (100 mg, 0.18 mmol, 1.0 equiv) ACN (3 mL) and TsOH·H₂O (107 mg, 0.56 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A21 (17.3 mg, 21%) as white solid. LCMS (ES, m/z): [M+H]⁺: 433.4. ¹H NMR (400 MHz, DMSO-d₆) δ 8.61 (d, J=8.5 Hz, 1H), 7.62 (d, J=8.1 Hz, 2H), 7.58-7.55 (m, 2H), 7.36 (d, J=8.0 Hz, 2H), 7.17 (d, J=8.3 Hz, 1H), 5.02 (q, J=8.2 Hz, 1H), 3.99 (dd, J=7.9, 3.7 Hz, 1H), 3.90-3.80 (m, 1H), 3.76-3.72 (m, 1H), 3.29 (s, 3H), 3.23-3.18 (m, 2H), 3.03 (dd, J=14.3, 3.8 Hz, 1H), 2.94 (t, J=7.4 Hz, 2H), 2.79-2.73 (m, 1H), 2.65-2.54 (m, 4H), 1.77-1.68 (m, 2H).

### Example 20: Synthesis of (2S)—N-[(1S)-1-cyano-2-(4-{1,3-dimethylpyrrolo[1,2-a]pyrazin-7-yl}phenyl)ethyl]-1,4-oxazocane-2-carboxamide (Compound A27)

### Step 1. Synthesis of tert-butyl N-[(1S)-1-cyano-2-(4-{1,3-dimethylpyrrolo[1,2-a]pyrazin-7-yl}phenyl)ethyl]carbamate

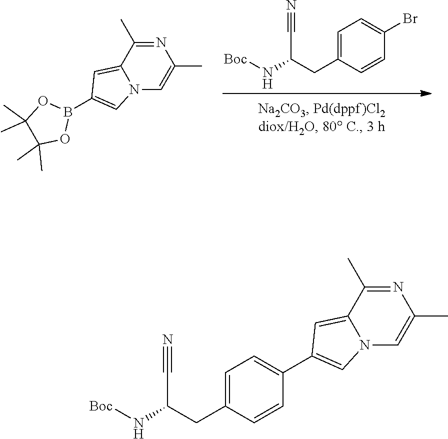

To a stirred solution of tert-butyl N-[(1S)-2-(4-bromophenyl)-1-cyanoethyl]carbamate (350 mg, 1.07 mmol, 1.0 equiv) and 1,3-dimethyl-7-(4,4,5,5-tetramethyl-1,3,2-dioxaborolan-2-yl)pyrrolo[1,2-a]pyrazine (380 mg, 1.39 mmol, 1.3 equiv) in dioxane (6 mL) and H₂O (0.6 mL) were added Na₂CO₃ (228 mg, 2.15 mmol, 2.0 equiv) and Pd(dppf)Cl₂ (78 mg, 0.10 mmol, 0.1 equiv). The resulting mixture was stirred for 3 h at 80° C. under nitrogen atmosphere. The mixture was allowed to cool down to room temperature. The resulting mixture was filtered, the filter cake was washed with EA (10 mL). The filtrate was concentrated under reduced pressure. The residue was purified by silica gel column chromatography, eluted with PE/THF (2:1) to afford tert-butyl N-[(1S)-1-cyano-2-(4-{1,3-dimethylpyrrolo[1,2-a]pyrazin-7-yl}phenyl)ethyl]carbamate (300 mg, 71.4%) as colorless oil. LCMS (ES, m/z): [M+H]⁺: 391.

### Step 2. Synthesis of (2S)-2-amino-3-(4-{1,3-dimethylpyrrolo[1,2-a]pyrazin-7-yl}phenyl)propanenitrile

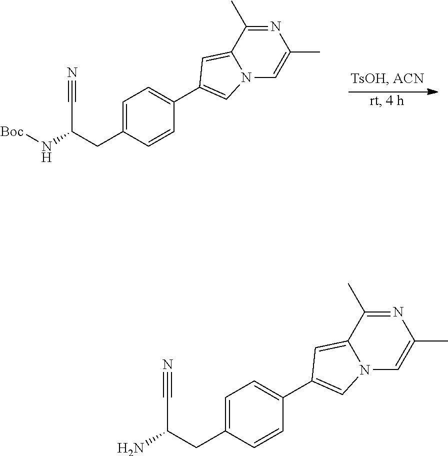 [SMILES: Cc1cn2cc(-c3ccc(C[C@@H](C#N)NC(=O)OC(C)(C)C)cc3)cc2c(C)n1 ; Cc1cn2cc(-c3ccc(C[C@H](N)C#N)cc3)cc2c(C)n1]

To a stirred solution of tert-butyl N-[(1S)-1-cyano-2-(4-{1,3-dimethylpyrrolo[1,2-a]pyrazin-7-yl}phenyl)ethyl]carbamate (280 mg, 0.72 mmol, 1.0 equiv) in ACN (5 mL) was added TsOH·H₂O (409 mg, 2.15 mmol, 3.0 equiv) at room temperature. The resulting mixture was stirred for 4 h at room temperature. The mixture was acidified to pH 8 with saturated NaHCO₃ (aq.). The aqueous layer was extracted with EtOAc (3×30 mL). The combined organic layer was dried over anhydrous Na₂SO₄. After filtration, the filtrate was concentrated under reduced pressure. This resulted in (2S)-2-amino-3-(4-{1,3-dimethylpyrrolo[1,2-a]pyrazin-7-yl}phenyl)propanenitrile (180 mg, 86.5%) as light yellow oil. LCMS (ES, m/z): [M+H]⁺: 291.

### Step 3. Synthesis of tert-butyl (2S)-2-{[(1S)-1-cyano-2-(4-{1,3-dimethylpyrrolo[1,2-a]pyrazin-7-yl}phenyl)ethyl]carbamoyl}-1,4-oxazocane-4-carboxylate

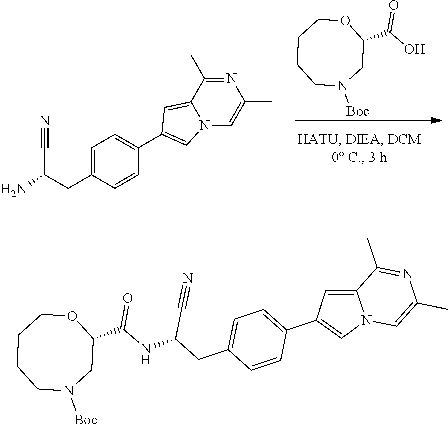 [SMILES: Cc1cn2cc(-c3ccc(C[C@H](N)C#N)cc3)cc2c(C)n1 ; CC(C)(C)OC(=O)N1CCCCO[C@H](C(=O)O)C1 ; Cc1cn2cc(-c3ccc(C[C@@H](C#N)NC(=O)[C@@H]4CN(C(=O)OC(C)(C)C)CCCCO4)cc3)cc2c(C)n1]

To a stirred mixture of (2S)-2-amino-3-(4-{1,3-dimethylpyrrolo[1,2-a]pyrazin-7-yl}phenyl)propanenitrile (102 mg, 0.35 mmol, 1.3 equiv), (2S)-4-(tert-butoxycarbonyl)-1,4-oxazocane-2-carboxylic acid (70 mg, 0.27 mmol, 1.0 equiv) and DIEA (70 mg, 0.54 mmol, 2.0 equiv) in DCM (5 mL) were added HATU (123 mg, 0.32 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S)-2-{[(1S)-1-cyano-2-(4-{1,3-dimethylpyrrolo[1,2-a]pyrazin-7-yl}phenyl)ethyl]carbamoyl}-1,4-oxazocane-4-carboxylate (110 mg, 76.6%) as light yellow oil. LCMS (ES, m/z): [M+H]⁺: 532.

### Step 4. Synthesis of (2S)—N-[(1S)-1-cyano-2-(4-{1,3-dimethylpyrrolo[1,2-a]pyrazin-7-yl}phenyl)ethyl]-1,4-oxazocane-2-carboxamide

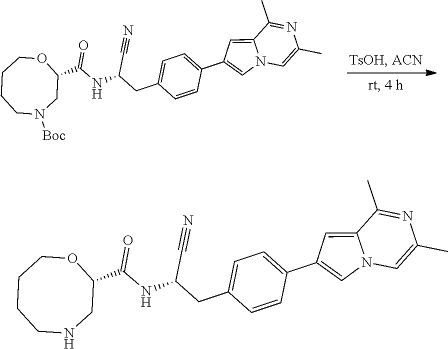 [SMILES: Cc1cn2cc(-c3ccc(C[C@@H](C#N)NC(=O)[C@@H]4CN(C(=O)OC(C)(C)C)CCCCO4)cc3)cc2c(C)n1 ; Cc1cn2cc(-c3ccc(C[C@@H](C#N)NC(=O)[C@@H]4CNCCCCO4)cc3)cc2c(C)n1]

Into a 25 mL round-bottom flask were added tert-butyl (2S)-2-{[(1S)-1-cyano-2-(4-{1,3-dimethylpyrrolo[1,2-a]pyrazin-7-yl}phenyl)ethyl]carbamoyl}-1,4-oxazocane-4-carboxylate (110 mg, 0.21 mmol, 1.0 equiv), TsOH·H₂O (118 mg, 0.62 mmol, 3.0 equiv) and ACN (4 mL) at room temperature. The resulting mixture was stirred for 4 h at room temperature. The reaction solution was purified by reversed-phase flash chromatography with the following conditions: column, C18 silica gel-120 g; mobile phase, MeCN in Water (0.1% NH₃·H₂O), 10% to 60% gradient in 12 min; detector, UV 254 nm. This resulted in Compound A27 (16 mg, 18%) as white solid. LCMS (ES, m/z): [M+H]⁺: 432.1. ¹H NMR (400 MHz, DMSO-d₆): δ 8.63 (d, J=8.6 Hz, 1H), 8.03 (s, 1H), 7.91 (s, 1H), 7.70 (d, J=7.9 Hz, 2H), 7.31 (d, J=7.9 Hz, 2H), 7.18 (s, 1H), 4.99 (q, J=8.1 Hz, 1H), 4.03-3.91 (m, 1H), 3.86 (dd, J=9.7, 2.9 Hz, 1H), 3.71-3.55 (m, 1H), 3.23-3.09 (m, 2H), 2.99-2.90 (m, 2H), 2.62-2.55 (m, 4H), 2.28 (s, 3H), 2.25-2.17 (m, 1H), 1.96-1.82 (m, 1H), 1.59-1.48 (m, 3H).

### Example 21: Synthesis of (2S)—N-[(1S)-1-cyano-2-(4-{1,3-dimethylpyrrolo[1,2-a]pyrazin-7-yl}-2-fluorophenyl)ethyl]-1,4-oxazocane-2-carboxamide (Compound A28)

### Step 1. Synthesis of 1,3-dimethyl-7-(4,4,5,5-tetramethyl-1,3,2-dioxaborolan-2-yl)pyrrolo[1,2-a]pyrazine

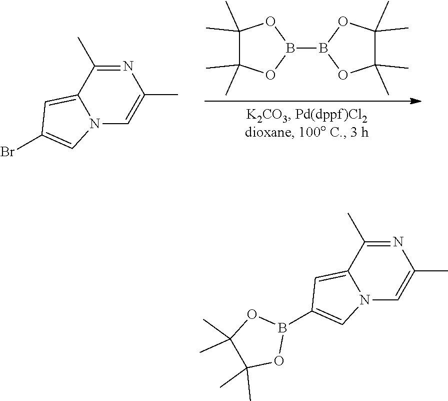

To a stirred solution of 7-bromo-1,3-dimethylpyrrolo[1,2-a]pyrazine (3.0 g, 13.328 mmol, 1.0 equiv) and bis(pinacolato)diboron (4.06 g, 15.994 mmol, 1.2 equiv) in dioxane (30 mL) were added AcOK (2.62 g, 26.656 mmol, 2.0 equiv) and Pd(dppf)Cl₂ (0.98 g, 1.333 mmol, 0.1 equiv). The resulting mixture was stirred for 3 h at 100° C. under nitrogen atmosphere. The mixture was allowed to cool down to room temperature. The resulting mixture was concentrated under reduced pressure. The residue was purified by silica gel column chromatography, eluted with PE/EA (3:1) to afford 1,3-dimethyl-7-(4,4,5,5-tetramethyl-1,3,2-dioxaborolan-2-yl)pyrrolo[1,2-a]pyrazine (2.9 g, 79.95%) as a light brown oil. LCMS (ES) [M+H]⁺ m/z: 273.

### Step 2. Synthesis of tert-butyl N-[(1S)-1-cyano-2-(4-{1,3-dimethylpyrrolo[1,2-a]pyrazin-7-yl}-2-fluorophenyl)ethyl]carbamate

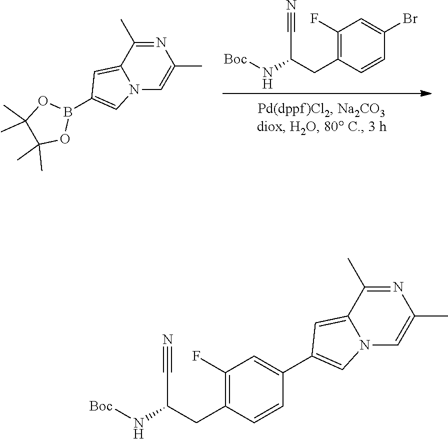

To a stirred solution of 1,3-dimethyl-7-(4,4,5,5-tetramethyl-1,3,2-dioxaborolan-2-yl)pyrrolo[1,2-a]pyrazine (1.03 g, 3.788 mmol, 1.3 equiv) and tert-butyl N-[(1S)-2-(4-bromo-2-fluorophenyl)-1-cyanoethyl]carbamate (1.0 g, 2.914 mmol, 1.0 equiv) in dioxane (15 mL) and H₂O (1.5 mL) were added Na₂CO₃ (0.62 g, 5.828 mmol, 2.0 equiv) and Pd(dppf)Cl₂ (0.21 g, 0.291 mmol, 0.1 equiv). The resulting mixture was stirred for 3 h at 80° C. under nitrogen atmosphere. The resulting mixture was concentrated under reduced pressure. The residue was purified by silica gel column chromatography, eluted with PE/THF (1:1) to afford tert-butyl N-[(1S)-1-cyano-2-(4-{1,3-dimethylpyrrolo[1,2-a]pyrazin-7-yl}-2-fluorophenyl)ethyl]carbamate (1.0 g, 84.02%) as an off-white solid. LCMS (ES) [M+H]⁺ m/z: 409.

### Step 3. Synthesis of (2S)-2-amino-3-(4-{1,3-dimethylpyrrolo[1,2-a]pyrazin-7-yl}-2-fluorophenyl)propanenitrile

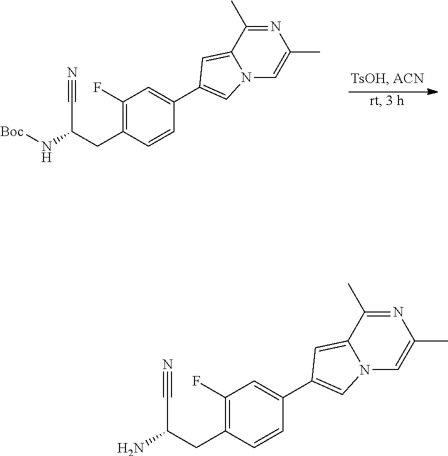 [SMILES: Cc1cn2cc(-c3ccc(C[C@@H](C#N)NC(=O)OC(C)(C)C)c(F)c3)cc2c(C)n1 ; Cc1cn2cc(-c3ccc(C[C@H](N)C#N)c(F)c3)cc2c(C)n1]

To a stirred solution of tert-butyl N-[(1S)-1-cyano-2-(4-{1,3-dimethylpyrrolo[1,2-a]pyrazin-7-yl}-2-fluorophenyl)ethyl]carbamate (1.0 g, 2.448 mmol, 1.0 equiv) in ACN (15 mL) was added TsOH (1.26 g, 7.344 mmol, 3.0 equiv). The resulting mixture was stirred for 5 h at room temperature. The mixture was basified to pH 8 with saturated NaHCO₃ (aq.). The resulting mixture was extracted with EtOAc (3×30 mL). The combined organic layers were washed with brine (50 mL), dried over anhydrous Na₂SO₄. After filtration, the filtrate was concentrated under reduced pressure. The residue was purified by silica gel column chromatography, eluted with PE/THF (1:2) to afford (2S)-2-amino-3-(4-{1,3-dimethylpyrrolo[1,2-a]pyrazin-7-yl}-2-fluorophenyl)propanenitrile (600 mg, 79.48%) as a light yellow solid. LCMS (ES) [M+H]⁺ m/z: 309.

### Step 4. Synthesis of tert-butyl (2S)-2-{[(1S)-1-cyano-2-(4-{1,3-dimethylpyrrolo[1,2-a]pyrazin-7-yl}-2-fluorophenyl)ethyl]carbamoyl}-1,4-oxazocane-4-carboxylate

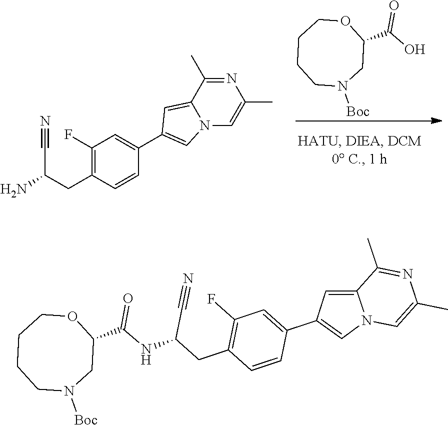 [SMILES: Cc1cn2cc(-c3ccc(C[C@H](N)C#N)c(F)c3)cc2c(C)n1 ; CC(C)(C)OC(=O)N1CCCCO[C@H](C(=O)O)C1 ; Cc1cn2cc(-c3ccc(C[C@@H](C#N)NC(=O)[C@@H]4CN(C(=O)OC(C)(C)C)CCCCO4)c(F)c3)cc2c(C)n1]

To a stirred solution of (2S)-2-amino-3-(4-{1,3-dimethylpyrrolo[1,2-a]pyrazin-7-yl}-2-fluorophenyl)propanenitrile (85 mg, 0.277 mmol, 1.2 equiv) and (2S)-4-(tert-butoxycarbonyl)-1,4-oxazocane-2-carboxylic acid (60 mg, 0.231 mmol, 1.0 equiv) in DCM (2 mL) were added DIEA (89 mg, 0.693 mmol, 3.0 equiv) and HATU (131 mg, 0.347 mmol, 1.5 equiv) in portions at 0° C. The resulting mixture was stirred for 2 h at 0° C. The resulting mixture was concentrated under reduced pressure. The residue was purified by silica gel column chromatography, eluted with PE/THF (3:1) to afford tert-butyl (2S)-2-{[(1S)-1-cyano-2-(4-{1,3-dimethylpyrrolo[1,2-a]pyrazin-7-yl}-2-fluorophenyl)ethyl]carbamoyl}-1,4-oxazocane-4-carboxylate (100 mg, 78.63%) as an off-white solid. LCMS (ES) [M+H]⁺ m/z: 550.

### Step 5. Synthesis of (2S)—N-[(1S)-1-cyano-2-(4-{1,3-dimethylpyrrolo[1,2-a]pyrazin-7-yl}-2-fluorophenyl)ethyl]-1,4-oxazocane-2-carboxamide

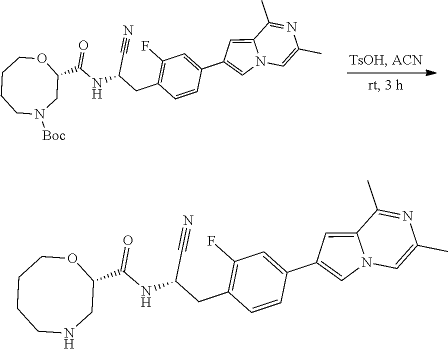 [SMILES: Cc1cn2cc(-c3ccc(C[C@@H](C#N)NC(=O)[C@@H]4CN(C(=O)OC(C)(C)C)CCCCO4)c(F)c3)cc2c(C)n1 ; Cc1cn2cc(-c3ccc(C[C@@H](C#N)NC(=O)[C@@H]4CNCCCCO4)c(F)c3)cc2c(C)n1]

To a stirred solution of tert-butyl (2S)-2-{[(1S)-1-cyano-2-(4-{1,3-dimethylpyrrolo[1,2-a]pyrazin-7-yl}-2-fluorophenyl)ethyl]carbamoyl}-1,4-oxazocane-4-carboxylate (100 mg, 0.182 mmol, 1.0 equiv) in ACN (2 mL) was added TsOH (93 mg, 0.546 mmol, 3.0 equiv). The resulting mixture was stirred for 4 h at room temperature. The mixture was purified by reversed-phase flash chromatography with the following conditions: column, C18 silica gel; mobile phase, MeCN in Water (0.1% NH₃·H₂O), 10% to 50% gradient in 15 min; detector, UV 254 nm. This resulted in Compound A28 (25 mg, 30.57%) as a white solid. LCMS (ES) [M+H]⁺ m/z: 450. ¹H NMR (400 MHz, DMSO-d₆) δ 8.71 (d, J=8.6 Hz, 1H), 8.10 (d, J=1.6 Hz, 1H), 7.90 (s, 1H), 7.65-7.54 (m, 2H), 7.37 (t, J=7.9 Hz, 1H), 7.26 (s, 1H), 5.02 (q, J=8.1 Hz, 1H), 3.97 (td, J=9.7, 9.0, 4.8 Hz, 1H), 3.87 (dd, J=9.6, 2.8 Hz, 1H), 3.67 (dd, J=11.6, 6.1 Hz, 1H), 3.26 (dd, J=13.7, 7.4 Hz, 1H), 3.16 (dd, J=13.8, 8.6 Hz, 1H), 2.96 (ddd, J=18.2, 11.4, 3.9 Hz, 2H), 2.65-2.57 (m, 1H), 2.57 (s, 3H), 2.28 (s, 3H), 2.32-2.22 (m, 1H), 1.94-1.82 (m, 1H), 1.56-1.50 (m, 3H).

### Example 22: Synthesis of (2S)—N-[(1S)-1-cyano-2-(5-{1,3-dimethylpyrrolo[1,2-a]pyrazin-7-yl}thieno[3,2-b]thiophen-2-yl)ethyl]-1,4-oxazocane-2-carboxamide (Compound A30) and (2S)—N-[(1R)-1-cyano-2-(5-{1,3-dimethylpyrrolo[1,2-a]pyrazin-7-yl}thieno[3,2-b]thiophen-2-yl)ethyl]-1,4-oxazocane-2-carboxamide (Compound A31)

### Step 1. Synthesis of 3-(5-{1,3-dimethylpyrrolo[1,2-a]pyrazin-7-yl}thieno[3,2-b]thiophen-2-yl)-2-[(diphenylmethylidene)amino]propanenitrile

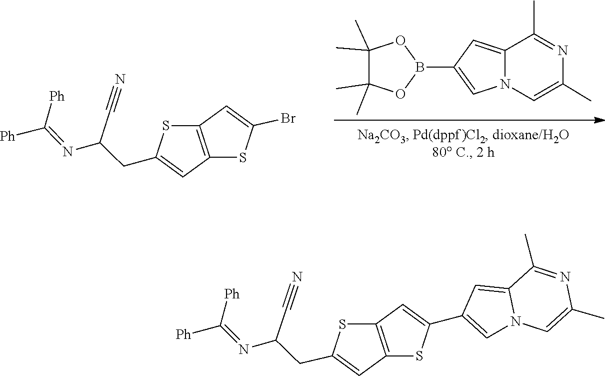

To a stirred solution of 3-{5-bromothieno[3,2-b]thiophen-2-yl}-2-[(diphenylmethylidene)amino]propanenitrile (400 mg, 0.89 mmol, 1.0 equiv) and 1,3-dimethyl-7-(4,4,5,5-tetramethyl-1,3,2-dioxaborolan-2-yl)pyrrolo[1,2-a]pyrazine (289 mg, 1.06 mmol, 1.2 equiv) in dioxane (5 mL) and H₂O (0.6 mL) were added Na₂CO₃ (187 mg, 1.77 mmol, 2.0 equiv) and Pd(dppf)Cl₂ (64 mg, 0.09 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (3:1) to afford 3-(5-{1,3-dimethylpyrrolo[1,2-a]pyrazin-7-yl}thieno[3,2-b]thiophen-2-yl)-2-[(diphenylmethylidene)amino]propanenitrile (250 mg, 54.6%) as light yellow solid. LCMS (ES) [M+1]⁺ m/z: 517.

### Step 2. Synthesis of 2-amino-3-(5-{1,3-dimethylpyrrolo[1,2-a]pyrazin-7-yl}thieno[3,2-b]thiophen-2-yl)propanenitrile

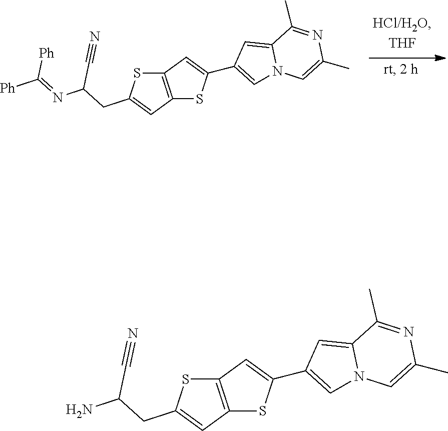 [SMILES: Cc1cn2cc(-c3cc4sc(CC(C#N)N=C(c5ccccc5)c5ccccc5)cc4s3)cc2c(C)n1 ; Cc1cn2cc(-c3cc4sc(CC(N)C#N)cc4s3)cc2c(C)n1]

To a stirred solution of 3-(5-{1,3-dimethylpyrrolo[1,2-a]pyrazin-7-yl}thieno[3,2-b]thiophen-2-yl)-2-[(diphenylmethylidene)amino]propanenitrile (250 mg, 0.48 mmol, 1.0 equiv) in THF (10 mL) and H₂O (1 mL) were added HCl (1 M) (0.6 mL). The resulting mixture was stirred for 2 h at room temperature. The resulting mixture was concentrated under reduced pressure. The residue was diluted with water (20 mL), extracted with ethyl ether (2×10 mL). The aqueous layer was basified to pH 10 with NaOH (aq.) (1 M), extracted with CH₂Cl₂ (3×10 mL). The combined organic layer was washed with brine (10 mL), dried over anhydrous Na₂SO₄. After filtration, the filtrate was concentrated under reduced pressure. This resulted in 2-amino-3-(5-{1,3-dimethylpyrrolo[1,2-a]pyrazin-7-yl}thieno[3,2-b]thiophen-2-yl)propanenitrile (140 mg, 82%) as light yellow solid. LCMS (ES) [M+1]⁺ m/z: 353.

### Step 3. Synthesis of tert-butyl (2S)-2-{[1-cyano-2-(5-{1,3-dimethylpyrrolo[1,2-a]pyrazin-7-yl}thieno[3,2-b]thiophen-2-yl)ethyl]carbamoyl}-1,4-oxazocane-4-carboxylate

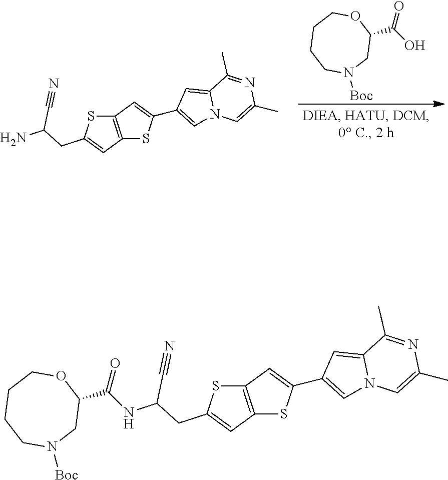 [SMILES: Cc1cn2cc(-c3cc4sc(CC(N)C#N)cc4s3)cc2c(C)n1 ; CC(C)(C)OC(=O)N1CCCCO[C@H](C(=O)O)C1 ; Cc1cn2cc(-c3cc4sc(CC(C#N)NC(=O)[C@@H]5CN(C(=O)OC(C)(C)C)CCCCO5)cc4s3)cc2c(C)n1]

To a stirred solution of 2-amino-3-(5-{1,3-dimethylpyrrolo[1,2-a]pyrazin-7-yl}thieno[3,2-b]thiophen-2-yl)propanenitrile (134 mg, 0.38 mmol, 1.1 equiv) and (2S)-4-(tert-butoxycarbonyl)-1,4-oxazocane-2-carboxylic acid (90 mg, 0.35 mmol, 1.0 equiv) in DCM (2 mL) were added DIEA (134 mg, 1.04 mmol, 3.0 equiv) and HATU (158 mg, 0.42 mmol, 1.2 equiv) in portions at 0° C. under nitrogen atmosphere. The resulting mixture was stirred for 2 h at 0° C. Concentrated under reduced pressure to remove the solvent, the residue was purified by silica gel column chromatography, eluted with PE/THF (1:1) to afford tert-butyl (2S)-2-{[1-cyano-2-(5-{1,3-dimethylpyrrolo[1,2-a]pyrazin-7-yl}thieno[3,2-b]thiophen-2-yl)ethyl]carbamoyl}-1,4-oxazocane-4-carboxylate (180 mg, 87%) as white solid. LCMS (ES) [M+1]⁺ m/z: 594.

### Step 4. Synthesis of (2S)—N-[l-cyano-2-(5-{1,3-dimethylpyrrolo[1,2-a]pyrazin-7-yl}thieno[3,2-b]thiophen-2-yl)ethyl]-1,4-oxazocane-2-carboxamide

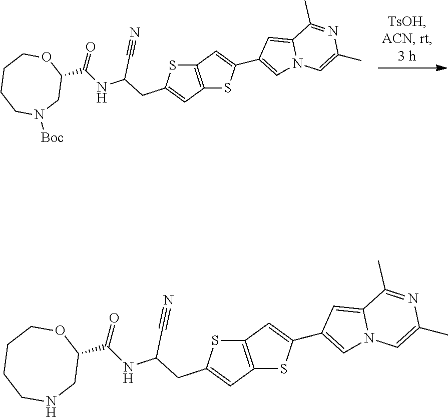 [SMILES: Cc1cn2cc(-c3cc4sc(CC(C#N)NC(=O)[C@@H]5CN(C(=O)OC(C)(C)C)CCCCO5)cc4s3)cc2c(C)n1 ; Cc1cn2cc(-c3cc4sc(CC(C#N)NC(=O)[C@@H]5CNCCCCO5)cc4s3)cc2c(C)n1]

To a stirred solution of tert-butyl (2S)-2-{[1-cyano-2-(5-{1,3-dimethylpyrrolo[1,2-a]pyrazin-7-yl}thieno[3,2-b]thiophen-2-yl)ethyl]carbamoyl}-1,4-oxazocane-4-carboxylate (180 mg, 0.30 mmol, 1.0 equiv) in ACN (3 mL) was added TsOH (156 mg, 0.90 mmol, 3.0 equiv). The resulting mixture was stirred for 3 h at room temperature. The reaction solution was purified by Prep-HPLC with the following conditions (Column, XBridge Prep C18 OBD Column, 19*150 mm, 5 um; mobile phase, Water (0.1% NH₃H₂O) and ACN (10% Phase B up to 80% in 20 min); Detector, UV 254 nm. This resulted in (2S)—N-[1-cyano-2-(5-{1,3-dimethylpyrrolo[1,2-a]pyrazin-7-yl}thieno[3,2-b]thiophen-2-yl)ethyl]-1,4-oxazocane-2-carboxamide (90 mg, 60%) as white solid. LCMS (ES) [M+1]⁺ m/z: 494.

### Step 5. Synthesis of (2S)—N-[(1S)-1-cyano-2-(5-{1,3-dimethylpyrrolo[1,2-a]pyrazin-7-yl}thieno[3,2-b]thiophen-2-yl)ethyl]-1,4-oxazocane-2-carboxamide and (2S)—N-[(1R)-1-cyano-2-(5-{1,3-dimethylpyrrolo[1,2-a]pyrazin-7-yl}thieno[3,2-b]thiophen-2-yl)ethyl]-1,4-oxazocane-2-carboxamide

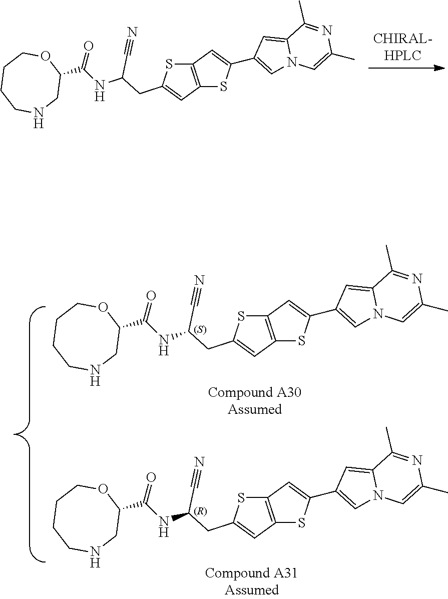

The product (2S)—N-[1-cyano-2-(5-{1,3-dimethylpyrrolo[1,2-a]pyrazin-7-yl}thieno[3,2-b]thiophen-2-yl)ethyl]-1,4-oxazocane-2-carboxamide (80 mg, 0.16 mmol, 1.0 equiv) was purified by Prep-CHIRAL-HPLC with the following conditions: Column: CHIRALPAK IC, 2*25 cm, 5 m; Mobile Phase A: Hex:DCM=1:1-HPLC, Mobile Phase B: EtOH (0.1% 2M NH₃-MeOH); Flow rate: 25 mL/min; Gradient: isocratic 20; Wave Length: 254 nm; RT1(min): 6.1; RT2(min): 8.1; Sample Solvent: EtOH:DCM=1:1-HPLC; Injection Volume: 1.5 mL; Number Of Runs: 6. This resulted in Compound A30 (25 mg, 31%) and Compound A31 (25 mg, 31%) as white solid.

Compound A30: LCMS (ES) [M+H]⁺ m/z: 494.0. ¹H NMR (300 MHz, DMSO-d₆) δ 8.74 (d, J=8.6 Hz, 1H), 7.96 (d, J=1.6 Hz, 1H), 7.93 (s, 1H), 7.68 (s, 1H), 7.30 (s, 1H), 7.05 (t, J=1.4 Hz, 1H), 5.06-4.97 (m, 1H), 4.08-3.85 (m, 2H), 3.72-3.65 (m, 1H), 3.57-3.36 (m, 2H), 3.07-2.89 (m, 2H), 2.64-2.57 (m, 4H), 2.37-2.24 (m, 4H), 1.95-1.83 (m, 1H), 1.61-1.47 (m, 3H).

Compound A31: LCMS (ES) [M+H]⁺ m/z: 494.0. ¹H NMR (300 MHz, DMSO-d₆) δ 8.77 (d, J=8.4 Hz, 1H), 7.96 (d, J=1.6 Hz, 1H), 7.93 (s, 1H), 7.69 (s, 1H), 7.31 (s, 1H), 7.05 (t, J=1.3 Hz, 1H), 5.04-4.90 (m, 1H), 4.04-3.95 (m, 1H), 3.83 (dd, J=9.8, 2.8 Hz, 1H), 3.68-3.60 (m, 1H), 3.57-3.42 (m, 2H), 3.12 (dd, J=14.0, 2.9 Hz, 1H), 3.06-2.94 (m, 1H), 2.66-2.56 (m, 4H), 2.48-2.40 (m, 1H), 2.28 (d, J=1.0 Hz, 3H), 1.97-1.84 (m, 1H), 1.60-1.45 (m, 3H).

### Example 23: Synthesis of (2S,7R)—N-[(1S)-1-cyano-2-(4-{1,3-dimethylpyrrolo[1,2-a]pyrazin-7-yl}phenyl)ethyl]-7-methoxy-1,4-oxazocane-2-carboxamide (Compound A32)

### Step 1. Synthesis of tert-butyl (2S,7R)-2-{[(1S)-1-cyano-2-(4-{1,3-dimethylpyrrolo[1,2-a]pyrazin-7-yl}phenyl)ethyl]carbamoyl}-7-methoxy-1,4-oxazocane-4-carboxylate

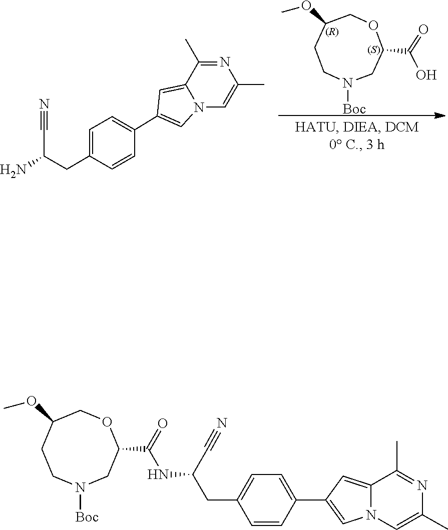

To a stirred solution of (2S,7R)-4-(tert-butoxycarbonyl)-7-methoxy-1,4-oxazocane-2-carboxylic acid (90 mg, 0.31 mmol, 1.0 equiv), (2S)-2-amino-3-(4-{1,3-dimethylpyrrolo[1,2-a]pyrazin-7-yl}phenyl)propanenitrile (90 mg, 0.31 mmol, 1.0 equiv) and DIEA (120 mg, 0.93 mmol, 3.0 equiv) in DCM (3 mL) was added HATU (142 mg, 0.37 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S,7R)-2-{[(1S)-1-cyano-2-(4-{1,3-dimethylpyrrolo[1,2-a]pyrazin-7-yl}phenyl)ethyl]carbamoyl}-7-methoxy-1,4-oxazocane-4-carboxylate (130 mg, 74.4%) as a yellow semi-solid. LCMS (ES, m/z): [M+H]⁺: 562.

### Step 2. Synthesis of (2S,7R)—N-[(1S)-1-cyano-2-(4-{1,3-dimethylpyrrolo[1,2-a]pyrazin-7-yl}phenyl)ethyl]-7-methoxy-1,4-oxazocane-2-carboxamide

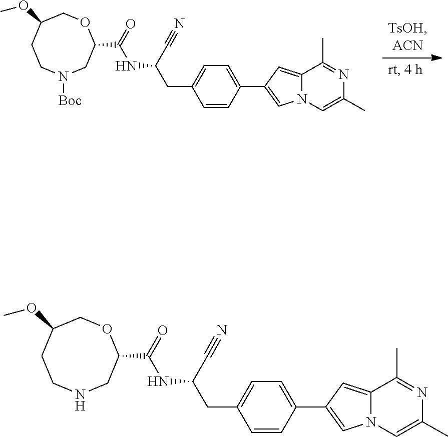

Into a 25 mL round-bottom flask were added tert-butyl (2S,7R)-2-{[(1S)-1-cyano-2-(4-{1,3-dimethylpyrrolo[1,2-a]pyrazin-7-yl}phenyl)ethyl]carbamoyl}-7-methoxy-1,4-oxazocane-4-carboxylate (120 mg, 0.21 mmol, 1.0 equiv), ACN (3 mL) and TsOH·H₂O (122 mg, 0.64 mmol, 3.0 equiv) at room temperature. The resulting mixture was stirred for 4 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A32 (20.5 mg, 21%) as a white solid. LCMS (ES, m/z): [M+H]⁺: 462.1. ¹H NMR (400 MHz, DMSO-d₆) δ 8.63 (d, J=8.5 Hz, 1H), 8.03 (d, J=1.6 Hz, 1H), 7.91 (s, 1H), 7.70 (d, J=7.8 Hz, 2H), 7.32 (d, J=7.9 Hz, 2H), 7.17 (s, 1H), 5.00 (q, J=8.2 Hz, 1H), 4.10 (dd, J=11.4, 4.4 Hz, 1H), 3.91 (dd, J=9.0, 3.0 Hz, 1H), 3.58 (dd, J=11.5, 8.6 Hz, 1H), 3.40-3.34 (m, 1H), 3.26 (s, 3H), 3.22-3.14 (m, 2H), 3.03-2.96 (m, 2H), 2.57 (s, 3H), 2.49-2.46 (m, 1H), 2.33 (dd, J=14.4, 9.0 Hz, 1H), 2.28 (s, 3H), 2.00-1.84 (m, 1H), 1.70-1.51 (m, 1H).

### Example 24: Synthesis of (2S,7R)—N—((S)-1-cyano-2-(4-(5,6,7,8-tetrahydro-1,5-naphthyridin-2-yl)phenyl)ethyl)-7-methoxy-1,4-oxazocane-2-carboxamide (Compound A33)

### Step 1. Synthesis of tert-butyl (S)-(1-cyano-2-(4-(5,6,7,8-tetrahydro-1,5-naphthyridin-2-yl)phenyl)ethyl)carbamate

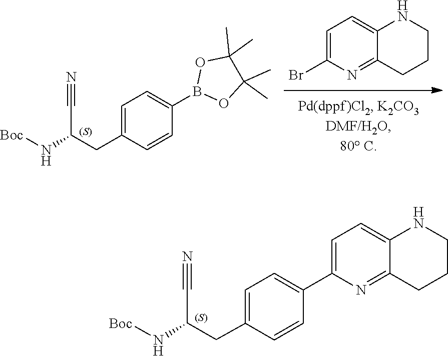

To a mixture of tert-butyl N-[(1S)-1-cyano-2-[4-(4,4,5,5-tetramethyl-1,3,2-dioxaborolan-2-yl)phenyl]ethyl]carbamate (600 mg, 1.61 mmol, 1.0 equiv), 6-bromo-1,2,3,4-tetrahydro-1,5-naphthyridine (341 mg, 1.61 mmol, 1.0 equiv), K₂CO₃ (446 mg, 3.22 mmol, 2.0 equiv) in DMF (5 mL), H₂O (0.5 mL) was added with Pd(dppf)Cl₂·CH₂Cl₂ (131 mg, 0.16 mmol, 0.1 equiv). The reaction was heated to 80° C. and stirred for 3 h under nitrogen atmosphere. The reaction was cooled to room temperature, diluted with water (15 mL), extracted with ethyl acetate (30 mL×2). The combined organic phase was washed with brine (15 mL×3), dried over anhydrous sodium sulfate. After filtration, the filtrate was concentrated under vacuum. The residue was purified by silica gel column chromatography, eluted with PE/EA (1:1) to afford tert-butyl N-[(1S)-1-cyano-2-[4-(5,6,7,8-tetrahydro-1,5-naphthyridin-2-yl)phenyl]ethyl]carbamate (450 mg, 74%) as a yellow oil. LCMS (ES) [M+1]⁺ m/z: 379.

### Step 2. Synthesis of (S)-2-amino-3-(4-(5,6,7,8-tetrahydro-1,5-naphthyridin-2-yl)phenyl)propanenitrile

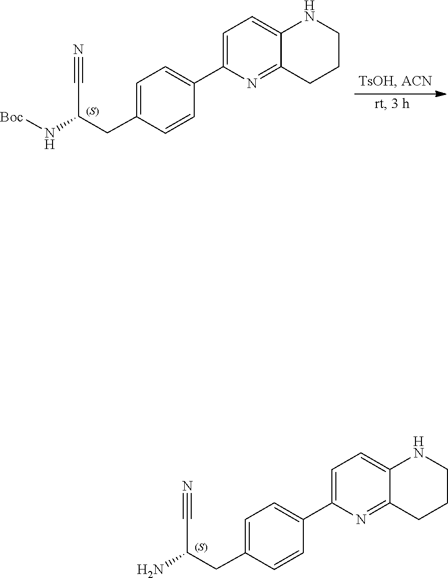

A solution of tert-butyl N-[(1S)-1-cyano-2-[4-(5,6,7,8-tetrahydro-1,5-naphthyridin-2-yl)phenyl]ethyl]carbamate (110 mg, 0.29 mmol, 1.0 equiv) and TsOH·H₂O (166 mg, 0.87 mmol, 3.0 equiv) in CH₃CN (5 mL) was stirred for 3 h at room temperature. The mixture was basified to pH 8 with saturated NaHCO₃(aq.). The aqueous layer was extracted with EtOAc (15 mL×2). The combined organic layer was dried over anhydrous Na₂SO₄. After filtration, the filtrate was concentrated under reduced pressure. The residue was purified by silica gel column chromatography, eluted with PE/THF (1:4) to afford (2S)-2-amino-3-[4-(5,6,7,8-tetrahydro-1,5-naphthyridin-2-yl)phenyl]propanenitrile (80 mg, 98%) as a yellow solid. LCMS (ES) [M+1]⁺ m/z: 279.

### Step 3. Synthesis of tert-butyl (2S,7R)-2-(((S)-1-cyano-2-(4-(5,6,7,8-tetrahydro-1,5-naphthyridin-2-yl)phenyl)ethyl)carbamoyl)-7-methoxy-1,4-oxazocane-4-carboxylate

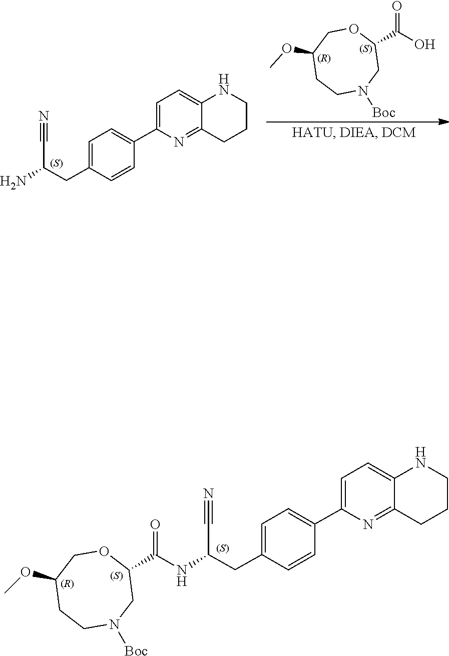

Into a 8 mL vial were added (2S)-2-amino-3-[4-(5,6,7,8-tetrahydro-1,5-naphthyridin-2-yl)phenyl]propanenitrile (67 mg, 0.24 mmol, 1.0 equiv), (2S,7R)-4-(tert-butoxycarbonyl)-7-methoxy-1,4-oxazocane-2-carboxylic acid (77 mg, 0.26 mmol, 1.1 equiv), DCM (3 mL), DIEA (93 mg, 0.72 mmol, 3.0 equiv). This was followed by the addition of HATU (110 mg, 0.29 mmol, 1.2 equiv) at 0° C. The resulting mixture was stirred for 2 h at 0° C. Concentrated under reduced pressure to remove the solvent, the residue was purified by silica gel column chromatography, eluted with PE/EA (1:3) to afford tert-butyl (2S,7R)-2-{[(1S)-1-cyano-2-[4-(5,6,7,8-tetrahydro-1,5-naphthyridin-2-yl)phenyl]ethyl]carbamoyl}-7-methoxy-1,4-oxazocane-4-carboxylate (74 mg, 56%) as a yellow solid. LCMS (ES) [M+1]⁺ m/z: 550.

### Step 4. Synthesis of (2S,7R)—N—((S)-1-cyano-2-(4-(5,6,7,8-tetrahydro-1,5-naphthyridin-2-yl)phenyl)ethyl)-7-methoxy-1,4-oxazocane-2-carboxamide

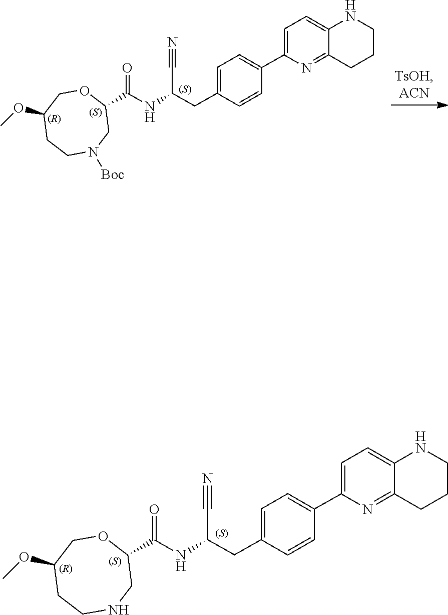

Into a 8 mL vial were added tert-butyl (2S,7R)-2-{[(1S)-1-cyano-2-[4-(5,6,7,8-tetrahydro-1,5-naphthyridin-2-yl)phenyl]ethyl]carbamoyl}-7-methoxy-1,4-oxazocane-4-carboxylate (74 mg, 0.14 mmol, 1.0 equiv), CH₃CN (2 mL) and TsOH·H₂O (77 mg, 0.41 mmol, 3.0 equiv) at room temperature. The resulting mixture was stirred for 2 h at room temperature. The reaction solution was purified by reverse phase flash with the following conditions: C18 silica column-120 g, mobile phase, MeCN in Water (0.1% NH₃·H₂O), 10% to 70% gradient in 10 min to afford Compound A33 (18 mg, 30%) as a white solid. LCMS (ES) [M+1]⁺ m/z: 450.2. ¹H NMR (300 MHz, DMSO-d₆) δ 8.62 (d, J=8.5 Hz, 1H), 7.88-7.80 (m, 2H), 7.46 (d, J=8.4 Hz, 1H), 7.33-7.23 (m, 2H), 6.83 (d, J=8.4 Hz, 1H), 6.00 (brs, 1H), 4.97 (q, J=8.1 Hz, 1H), 4.09 (dd, J=11.5, 4.1 Hz, 1H), 3.91 (dd, J=9.1, 3.1 Hz, 1H), 3.58 (dd, J=11.5, 8.5 Hz, 1H), 3.40-3.34 (m, 1H), 3.26 (s, 3H), 3.25-3.11 (m, 4H), 3.05-2.93 (m, 2H), 2.84 (t, J=6.4 Hz, 2H), 2.49-2.46 (m, 1H), 2.34 (dd, J=14.4, 9.1 Hz, 1H), 1.96-1.87 (m, 3H), 1.67-1.51 (m, 1H).

### Example 25: Synthesis of (2S,7R)—N-[(1S)-1-cyano-2-(5-{1,3-dimethylpyrrolo[1,2-a]pyrazin-7-yl}thiophen-2-yl)ethyl]-7-methoxy-1,4-oxazocane-2-carboxamide (Compound A34)

### Step 1. Synthesis of tert-butyl N-[(1S)-1-cyano-2-(5-{1,3-dimethylpyrrolo[1,2-a]pyrazin-7-yl}thiophen-2-yl)ethyl]carbamate

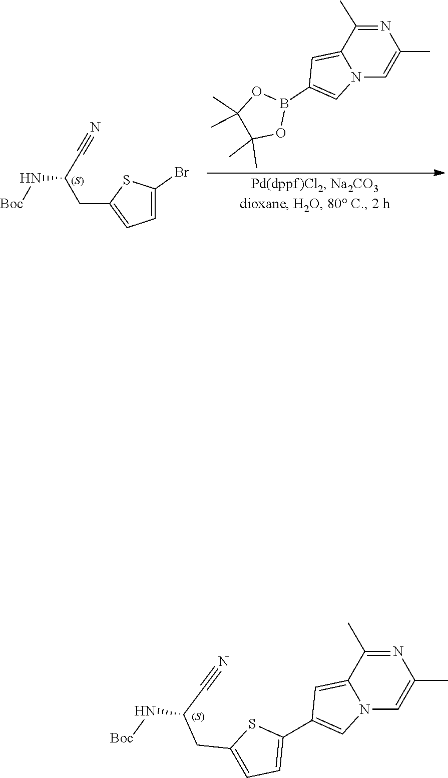

To a stirred solution of tert-butyl N-[(1S)-2-(5-bromothiophen-2-yl)-1-cyanoethyl]carbamate (400 mg, 1.208 mmol, 1.0 equiv) and 1,3-dimethyl-7-(4,4,5,5-tetramethyl-1,3,2-dioxaborolan-2-yl)pyrrolo[1,2-a]pyrazine (427 mg, 1.570 mmol, 1.3 equiv) in dioxane (8 mL) and H₂O (1 mL) were added Na₂CO₃ (255 mg, 2.416 mmol, 2.0 equiv) and Pd(dppf)Cl₂ (88 mg, 0.121 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (2:1) to afford tert-butyl N-[(1S)-1-cyano-2-(5-{1,3-dimethylpyrrolo[1,2-a]pyrazin-7-yl}thiophen-2-yl)ethyl]carbamate (270 mg, 56.3%) as a light yellow solid. LCMS (ES) [M+1]⁺ m/z: 397.

### Step 2. Synthesis of (2S)-2-amino-3-(5-{1,3-dimethylpyrrolo[1,2-a]pyrazin-7-yl}thiophen-2-yl)propanenitrile

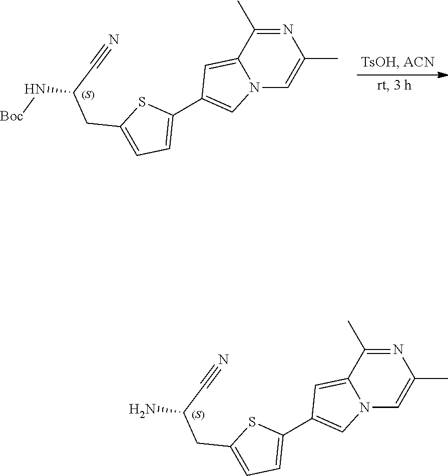

To a stirred solution of tert-butyl N-[(1S)-1-cyano-2-(5-{1,3-dimethylpyrrolo[1,2-a]pyrazin-7-yl}thiophen-2-yl)ethyl]carbamate (260 mg, 0.656 mmol, 1.0 equiv) in ACN (5 mL) was added TsOH (338 mg, 1.968 mmol, 3.0 equiv). The resulting mixture was stirred for 3 h at room temperature under nitrogen atmosphere. The resulting mixture was diluted with water (20 mL). The mixture was basified to pH 8 with saturated NaHCO₃ (aq.). The resulting mixture was extracted with EtOAc (3×20 mL). The combined organic layers were washed with brine (20 mL), dried over anhydrous Na₂SO₄. After filtration, the filtrate was concentrated under reduced pressure. The crude product (2S)-2-amino-3-(5-{1,3-dimethylpyrrolo[1,2-a]pyrazin-7-yl}thiophen-2-yl)propanenitrile (200 mg) was used in the next step directly without further purification. LCMS (ES) [M+1]⁺ m/z: 297.

### Step 3. Synthesis of tert-butyl (2S,7R)-2-{[(1S)-1-cyano-2-(5-{1,3-dimethylpyrrolo[1,2-a]pyrazin-7-yl}thiophen-2-yl)ethyl]carbamoyl}-7-methoxy-1,4-oxazocane-4-carboxylate

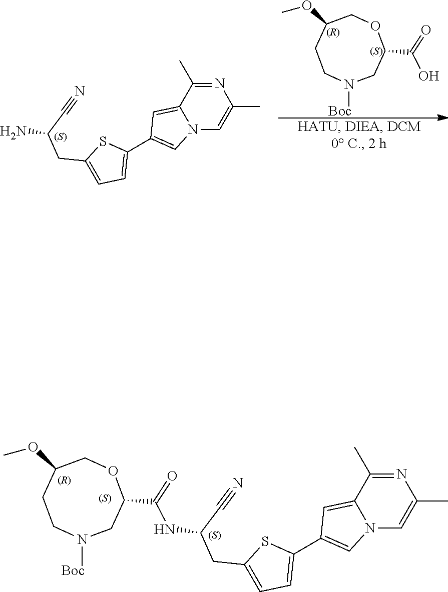

To a stirred solution of (2S)-2-amino-3-(5-{1,3-dimethylpyrrolo[1,2-a]pyrazin-7-yl}thiophen-2-yl)propanenitrile (114 mg, 0.388 mmol, 1.4 equiv) and (2S,7R)-4-(tert-butoxycarbonyl)-7-methoxy-1,4-oxazocane-2-carboxylic acid (80 mg, 0.277 mmol, 1.0 equiv) in DCM (2 mL) were added DIEA (107 mg, 0.831 mmol, 3.0 equiv) and HATU (126 mg, 0.332 mmol, 1.2 equiv) in portions at 0° C. under nitrogen atmosphere. The resulting mixture was stirred for 2 h at 0° C. The residue was purified by silica gel column chromatography, eluted with PE/THF (1:1) to afford tert-butyl (2S,7R)-2-{[(1S)-1-cyano-2-(5-{1,3-dimethylpyrrolo[1,2-a]pyrazin-7-yl}thiophen-2-yl)ethyl]carbamoyl}-7-methoxy-1,4-oxazocane-4-carboxylate (120 mg, 76.4%) as a white solid. LCMS (ES) [M+1]⁺ m/z: 568.

### Step 4. Synthesis of (2S,7R)—N-[(1S)-1-cyano-2-(5-{1,3-dimethylpyrrolo[1,2-a]pyrazin-7-yl}thiophen-2-yl)ethyl]-7-methoxy-1,4-oxazocane-2-carboxamide

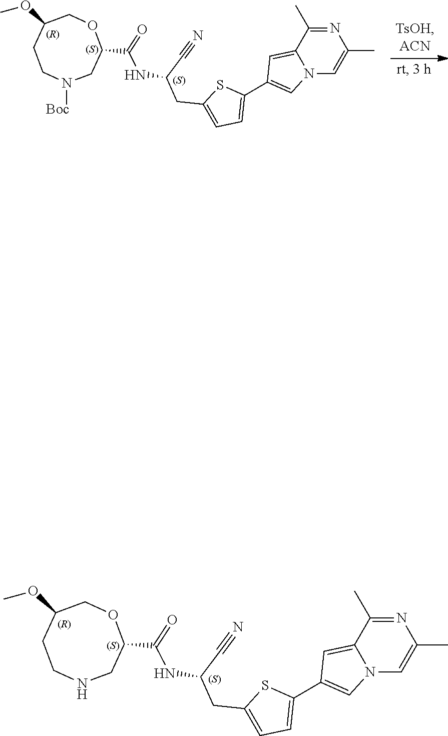

To a stirred solution of tert-butyl (2S,7R)-2-{[(1S)-1-cyano-2-(5-{1,3-dimethylpyrrolo[1,2-a]pyrazin-7-yl}thiophen-2-yl)ethyl]carbamoyl}-7-methoxy-1,4-oxazocane-4-carboxylate (100 mg, 0.176 mmol, 1.0 equiv) in ACN (2 mL) was added TsOH (91 mg, 0.528 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound A34 (35 mg, 42.4%) as a white solid. LCMS (ES) [M+1]⁺ m/z: 468. ¹H NMR (300 MHz, DMSO-d6) δ 8.72 (d, J=8.6 Hz, 1H), 7.91 (s, 1H), 7.86 (d, J=1.6 Hz, 1H), 7.23 (d, J=3.6 Hz, 1H), 6.98-6.91 (m, 2H), 5.05-4.91 (m, 1H), 4.13 (dd, J=11.4, 4.3 Hz, 1H), 3.95 (dd, J=9.1, 3.0 Hz, 1H), 3.62 (dd, J=11.5, 8.6 Hz, 1H), 3.51-3.30 (m, 3H), 3.26 (s, 3H), 3.11-2.95 (m, 2H), 2.55 (s, 3H), 2.44 (dd, J=14.3, 9.0 Hz, 1H), 2.27 (s, 3H), 2.01-1.88 (m, 1H), 1.68-1.52 (m, 1H).

### Example 26: Synthesis of (2S,7R)—N-[(1S)-1-cyano-2-(4-{1,3-dimethylpyrrolo[1,2-a]pyrazin-7-yl}-3-fluorophenyl)ethyl]-7-methoxy-1,4-oxazocane-2-carboxamide (Compound A35)

### Step 1. Synthesis of methyl (2S)-2-[(tert-butoxycarbonyl)amino]-3-(4-chloro-3-fluorophenyl)propanoate

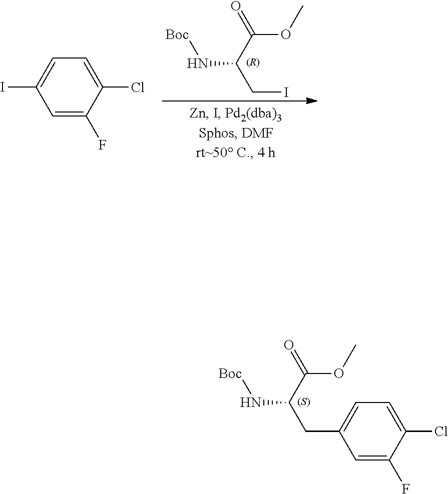

To a stirred mixture of Zn (3.4 g, 52.65 mmol, 3.0 equiv) and DMF (100 mL) was added I₂ (0.67 g, 2.63 mmol, 0.15 equiv) at room temperature under nitrogen atmosphere. The resulting mixture was stirred for 40 min at room temperature under nitrogen atmosphere. To the above mixture was added methyl (2R)-2-[(tert-butoxycarbonyl)amino]-3-iodopropanoate (5.78 g, 17.55 mmol, 1 equiv) and I₂ (0.67 g, 2.63 mmol, 0.15 equiv) at room temperature under nitrogen atmosphere. The resulting mixture was stirred for 80 min at room temperature under nitrogen atmosphere. To the above mixture was added 1-chloro-2-fluoro-4-iodobenzene (4.5 g, 17.55 mmol, 1.0 equiv), Pd₂(dba)₃ (0.80 g, 0.88 mmol, 0.05 equiv), SPhos (0.72 g, 1.76 mmol, 0.1 equiv) in DMF (100 mL) at room temperature under nitrogen atmosphere. The resulting mixture was stirred for additional 3 h at 50° C. The resulting mixture was cooled to room temperature, quenched with water (200 mL), extracted with EtOAc (2×200 mL). The combined organic layer was washed with brine (3×100 mL), dried over anhydrous Na₂SO₄. After filtration, the filtrate was concentrated under reduced pressure. The residue was purified by silica gel column chromatography, eluted with PE/EA (3:1) to afford methyl (2S)-2-[(tert-butoxycarbonyl)amino]-3-(4-chloro-3-fluorophenyl)propanoate (4.9 g, 84%) as a light yellow solid. LCMS (ES, m/z): [M+H]⁺: 332.

### Step 2. Synthesis of tert-butyl N-[(1S)-1-carbamoyl-2-(4-chloro-3-fluorophenyl)ethyl]carbamate

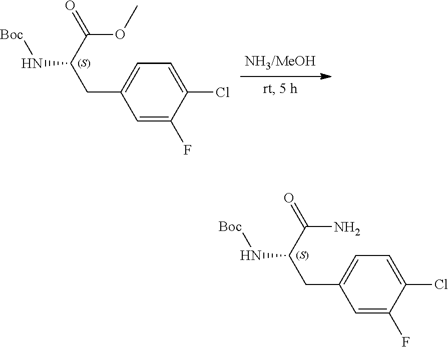

A solution of methyl (2S)-2-[(tert-butoxycarbonyl)amino]-3-(4-chloro-3-fluorophenyl)propanoate (4.4 g, 13.26 mmol, 1.0 equiv) and NH₃(g) in MeOH (44 mL) was stirred for 5 h at room temperature. The resulting mixture was concentrated under reduced pressure to remove the solvent. The residue was purified by trituration with PE/EA=10:1 (20 mL). The precipitated solid was collected by filtration and washed with PE (3×5 mL). This resulted in tert-butyl N-[(1S)-1-carbamoyl-2-(4-chloro-3-fluorophenyl)ethyl]carbamate (3.8 g, 90.4%) as a yellow solid. LCMS (ES, m/z): [M+H]⁺: 317.

### Step 3. Synthesis of tert-butyl N-[(1S)-2-(4-chloro-3-fluorophenyl)-1-cyanoethyl]carbamate

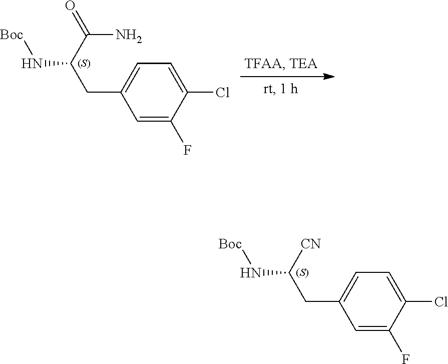

To a solution of tert-butyl N-[(1S)-1-carbamoyl-2-(4-chloro-3-fluorophenyl)ethyl]carbamate (1.5 g, 4.73 mmol, 1.0 equiv) and TEA (1.9 g, 18.94 mmol, 4.0 equiv) in DCM (15 mL) was added TFAA (2.0 g, 9.47 mmol, 2.0 equiv) at 0° C. After addition, the mixture was stirred for 1 h at room temperature. The reaction was quenched with water (30 mL), extracted with CH₂Cl₂ (3×50 mL). The combined organic layer was washed with brine (3×50 mL), dried over anhydrous Na₂SO₄. After filtration, the filtrate was concentrated under reduced pressure. The residue was purified by trituration with PE/EA=10:1 (20 mL). This resulted in tert-butyl N-[(1S)-2-(4-chloro-3-fluorophenyl)-1-cyanoethyl]carbamate (1.2 g, 85%) as a yellow solid. LCMS (ES, m/z): [M+H]⁺: 299.

### Step 4. Synthesis of tert-butyl N-[(1S)-1-cyano-2-(4-{1,3-dimethylpyrrolo[1,2-a]pyrazin-7-yl}-3-fluorophenyl)ethyl]carbamate

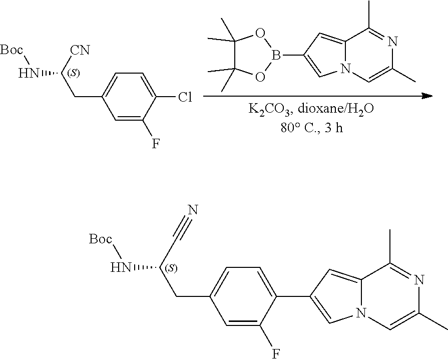

To a solution of tert-butyl N-[(1S)-2-(4-chloro-3-fluorophenyl)-1-cyanoethyl]carbamate (350 mg, 1.17 mmol, 1.0 equiv) and 1,3-dimethyl-7-(4,4,5,5-tetramethyl-1,3,2-dioxaborolan-2-yl)pyrrolo[1,2-a]pyrazine (414 mg, 1.52 mmol, 1.3 equiv) in 1,4-dioxane (5 mL), H₂O (0.5 mL), K₂CO₃ (324 mg, 2.34 mmol, 2.0 equiv), SPhos (96 mg, 0.23 mmol, 0.2 equiv) and Pd(OAc)₂ (26 mg, 0.11 mmol, 0.1 equiv) were added in sequence. The mixture was stirred for 3 h at 80° C. under nitrogen atmosphere. The reaction was cooled to room temperature, concentrated to remove the solvent. The residue was purified by silica gel column chromatography, eluted with PE/THF (1:1) to afford tert-butyl N-[(1S)-1-cyano-2-(4-{1,3-dimethylpyrrolo[1,2-a]pyrazin-7-yl}-3-fluorophenyl)ethyl]carbamate (420 mg, 87.7%) as a yellow solid. LCMS (ES, m/z): [M+H]⁺: 409.

### Step 5. Synthesis of (2S)-2-amino-3-(4-{1,3-dimethylpyrrolo[1,2-a]pyrazin-7-yl}-3-fluorophenyl)propanenitrile

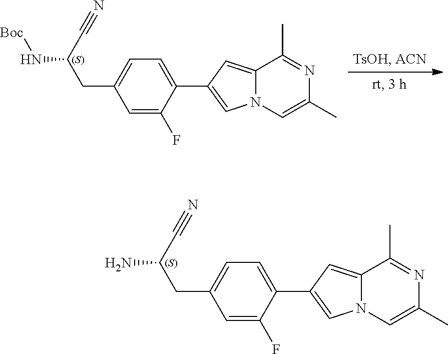

Into a 50 mL round-bottom flask were added tert-butyl N-[(1S)-1-cyano-2-(4-{1,3-dimethylpyrrolo[1,2-a]pyrazin-7-yl}-3-fluorophenyl)ethyl]carbamate (400 mg, 0.97 mmol, 1.0 equiv), ACN (12 mL) and TsOH·H₂O (506 mg, 2.93 mmol, 3.0 equiv) at room temperature. The resulting mixture was stirred for 3 h at room temperature. The mixture was basified to pH 8 with saturated NaHCO₃ (aq.). The resulting mixture was extracted with EtOAc (3×20 mL). The combined organic layer was washed with brine (3×20 mL), dried over anhydrous Na₂SO₄. After filtration, the filtrate was concentrated under reduced pressure. The residue was purified by silica gel column chromatography, eluted with PE/THF (1:2) to afford (2S)-2-amino-3-(4-{1,3-dimethylpyrrolo[1,2-a]pyrazin-7-yl}-3-fluorophenyl)propanenitrile (200 mg, 66%) as a white solid. LCMS (ES, m/z): [M+H]⁺: 309.

### Step 6. Synthesis of tert-butyl (2S,7R)-2-{[(1S)-1-cyano-2-(4-{1,3-dimethylpyrrolo[1,2-a]pyrazin-7-yl}-3-fluorophenyl)ethyl]carbamoyl}-7-methoxy-1,4-oxazocane-4-carboxylate

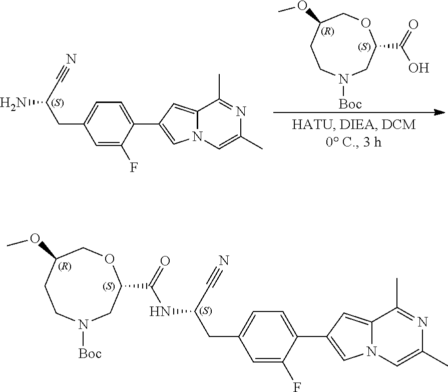

To a stirred solution of (2S,7R)-4-(tert-butoxycarbonyl)-7-methoxy-1,4-oxazocane-2-carboxylic acid (70 mg, 0.24 mmol, 1.0 equiv) and (2S)-2-amino-3-(4-{1,3-dimethylpyrrolo[1,2-a]pyrazin-7-yl}-3-fluorophenyl)propanenitrile (89 mg, 0.29 mmol, 1.2 equiv), DIEA (93 mg, 0.72 mmol, 3.0 equiv) in DCM (3 mL) was added HATU (110 mg, 0.29 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S,7R)-2-{[(1S)-1-cyano-2-(4-{1,3-dimethylpyrrolo[1,2-a]pyrazin-7-yl}-3-fluorophenyl)ethyl]carbamoyl}-7-methoxy-1,4-oxazocane-4-carboxylate (110 mg, 78.4%) as a white semi-solid. LCMS (ES, m/z): [M+H]⁺: 580.

### Step 7. Synthesis of (2S,7R)—N-[(1S)-1-cyano-2-(4-{1,3-dimethylpyrrolo[1,2-a]pyrazin-7-yl}-3-fluorophenyl)ethyl]-7-methoxy-1,4-oxazocane-2-carboxamide

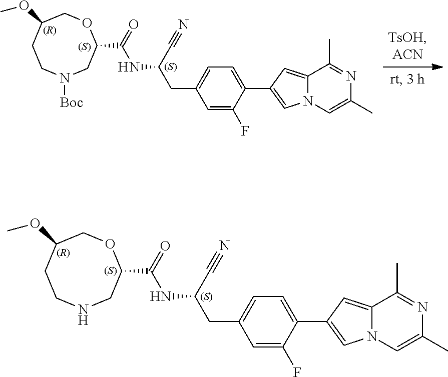

Into a 25 mL round-bottom flask were added tert-butyl (2S,7R)-2-{[(1S)-1-cyano-2-(4-{1,3-dimethylpyrrolo[1,2-a]pyrazin-7-yl}-3-fluorophenyl)ethyl]carbamoyl}-7-methoxy-1,4-oxazocane-4-carboxylate (110 mg, 0.19 mmol, 1.0 equiv), ACN (3 mL) and TsOH·H₂O (108 mg, 0.57 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 45% gradient in 15 min; detector, UV 254 nm. This resulted in Compound A35 (20.5 mg, 22.5%) as white solid. LCMS (ES, m/z): [M+H]⁺: 480.2. ¹H NMR (400 MHz, DMSO-d₆) δ 8.64 (d, J=8.7 Hz, 1H), 8.01 (t, J=2.1 Hz, 1H), 7.98 (s, 1H), 7.82 (t, J=8.1 Hz, 1H), 7.29-7.14 (m, 3H), 5.11-5.01 (m, 1H), 4.09 (dd, J=11.5, 4.4 Hz, 1H), 3.90 (dd, J=9.1, 3.0 Hz, 1H), 3.59 (dd, J=11.5, 8.6 Hz, 1H), 3.39-3.35 (m, 1H), 3.26 (s, 3H), 3.24-3.13 (m, 2H), 3.02-2.94 (m, 2H), 2.58 (s, 3H), 2.49-2.43 (m, 1H), 2.32-2.25 (m, 4H), 1.96-1.86 (m, 1H), 1.65-1.52 (m, 1H).

### Example 27: Synthesis of (2S,7R)—N-[(1S)-1-cyano-2-[5-(3-fluoro-1-methylindazol-6-yl)-1,3-thiazol-2-yl]ethyl]-7-methoxy-1,4-oxazocane-2-carboxamide (Compound A36)

### Step 1. Synthesis of 3-fluoro-1-methyl-6-(4,4,5,5-tetramethyl-1,3,2-dioxaborolan-2-yl)indazole

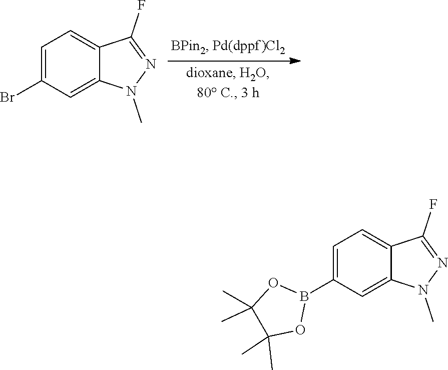 [SMILES: Cn1nc(F)c2ccc(Br)cc21 ; Cn1nc(F)c2ccc(B3OC(C)(C)C(C)(C)O3)cc21]

A mixture of 6-bromo-3-fluoro-1-methylindazole (240 mg, 1.05 mmol, 1.0 equiv) and bis(pinacolato)diboron (320 mg, 1.26 mmol, 1.2 equiv), KOAc (206 mg, 2.10 mmol, 2.0 equiv), Pd(dppf)Cl₂·CH₂Cl₂ (86 mg, 0.11 mmol, 0.1 equiv) in 1,4-dioxane (5 mL) was stirred for 3 h at 80° C. under nitrogen atmosphere. The mixture was cooled to room temperature, concentrated to remove the solvent. The residue was purified by silica gel column chromatography, eluted with PE/THF (10:1) to afford 3-fluoro-1-methyl-6-(4,4,5,5-tetramethyl-1,3,2-dioxaborolan-2-yl)indazole (250 mg, 86%) as a yellow oil. LCMS (ES, m/z): [M+H]⁺: 277.

### Step 2. Synthesis of tert-butyl N-[(1S)-1-cyano-2-[5-(3-fluoro-1-methylindazol-6-yl)-1,3-thiazol-2-yl]ethyl]carbamate

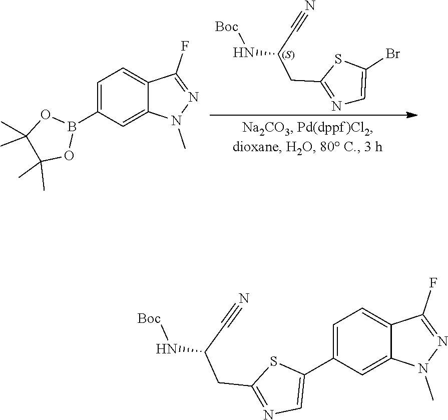

To a mixture of tert-butyl N-[(1S)-2-(5-bromo-1,3-thiazol-2-yl)-1-cyanoethyl]carbamate (300 mg, 0.90 mmol, 1.0 equiv) and 3-fluoro-1-methyl-6-(4,4,5,5-tetramethyl-1,3,2-dioxaborolan-2-yl)indazole (249 mg, 0.90 mmol, 1.0 equiv), Na₂CO₃ (191 mg, 1.80 mmol, 2.0 equiv) in 1,4-dioxane (5 mL), H₂O (0.5 mL), Pd(dppf)Cl₂·CH₂Cl₂ (74 mg, 0.09 mmol, 0.1 equiv) was added. The resulting mixture was heated to 80° C. and stirred for 3 h under nitrogen atmosphere. The reaction was cooled to room temperature, concentrated to remove the solvent. The residue was purified by silica gel column chromatography, eluted with PE/THF (2:1) to afford tert-butyl N-[(1S)-1-cyano-2-[5-(3-fluoro-1-methylindazol-6-yl)-1,3-thiazol-2-yl]ethyl]carbamate (320 mg, 89%) as a light yellow semi-solid. LCMS (ES, m/z): [M+H]⁺: 402.

### Step 3. Synthesis of (2S)-2-amino-3-[5-(3-fluoro-1-methylindazol-6-yl)-1,3-thiazol-2-yl]propanenitrile

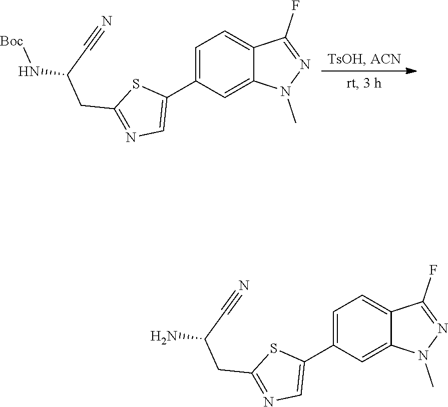 [SMILES: Cn1nc(F)c2ccc(-c3cnc(C[C@@H](C#N)NC(=O)OC(C)(C)C)s3)cc21 ; Cn1nc(F)c2ccc(-c3cnc(C[C@H](N)C#N)s3)cc21]

Into a 50 mL round-bottom flask were added tert-butyl N-[(1S)-1-cyano-2-[5-(3-fluoro-1-methylindazol-6-yl)-1,3-thiazol-2-yl]ethyl]carbamate (320 mg, 0.80 mmol, 1.0 equiv), ACN (12 mL) and TsOH·H₂O (514 mg, 2.98 mmol, 3.0 equiv) at room temperature. The resulting mixture was stirred for 3 h at room temperature. The mixture was basified to pH 8 with saturated K₂CO₃ (aq.). The resulting mixture was extracted with EtOAc (3×50 mL). The combined organic layer was washed with brine (3×50 mL), dried over anhydrous Na₂SO₄. After filtration, the filtrate was concentrated under reduced pressure. The residue was purified by silica gel column chromatography, eluted with PE/THF (1:1) to afford (2S)-2-amino-3-[5-(3-fluoro-1-methylindazol-6-yl)-1,3-thiazol-2-yl]propanenitrile (170 mg, 56.6%) as a light yellow solid. LCMS (ES, m/z): [M+H]⁺: 302.

### Step 4. Synthesis of tert-butyl (2S,7R)-2-{[(1S)-1-cyano-2-[5-(3-fluoro-1-methylindazol-6-yl)-1,3-thiazol-2-yl]ethyl]carbamoyl}-7-methoxy-1,4-oxazocane-4-carboxylate

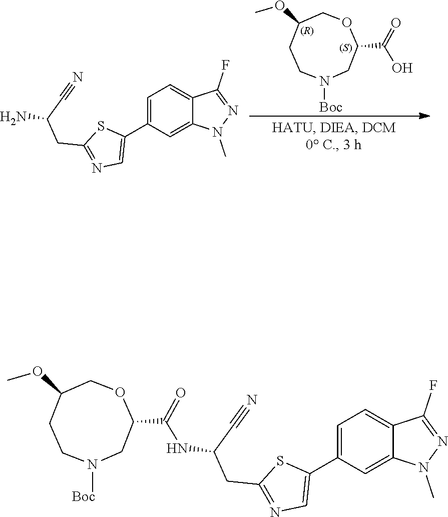

To a stirred solution of (2S,7R)-4-(tert-butoxycarbonyl)-7-methoxy-1,4-oxazocane-2-carboxylic acid (80 mg, 0.27 mmol, 1.0 equiv) and (2S)-2-amino-3-[5-(3-fluoro-1-methylindazol-6-yl)-1,3-thiazol-2-yl]propanenitrile (92 mg, 0.31 mmol, 1.1 equiv), DIEA (107 mg, 0.83 mmol, 3.0 equiv) in DCM (3 mL) was added HATU (126 mg, 0.33 mmol, 1.2 equiv) in portions at 0° C. The resulting mixture was stirred for 3 h at 0° C. Concentrated to remove the solvent, the residue was purified by silica gel column chromatography, eluted with PE/THF (1:1) to afford tert-butyl (2S,7R)-2-{[(1S)-1-cyano-2-[5-(3-fluoro-1-methylindazol-6-yl)-1,3-thiazol-2-yl]ethyl]carbamoyl}-7-methoxy-1,4-oxazocane-4-carboxylate (140 mg, 90%) as a white semi-solid. LCMS (ES, m/z): [M+H]⁺: 573.

### Step 5. Synthesis of (2S,7R)—N-[(1S)-1-cyano-2-[5-(3-fluoro-1-methylindazol-6-yl)-1,3-thiazol-2-yl]ethyl]-7-methoxy-1,4-oxazocane-2-carboxamide

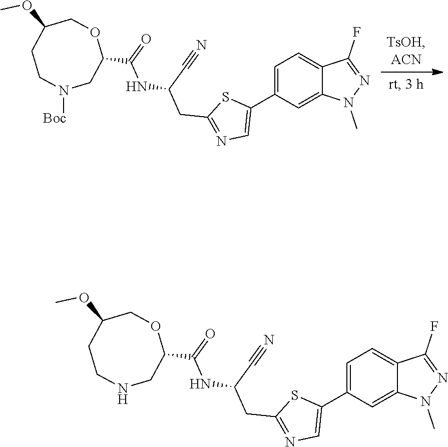 [SMILES: CO[C@@H]1CCN(C(=O)OC(C)(C)C)C[C@@H](C(=O)N[C@H](C#N)Cc2ncc(-c3ccc4c(F)nn(C)c4c3)s2)OC1 ; CO[C@@H]1CCNC[C@@H](C(=O)N[C@H](C#N)Cc2ncc(-c3ccc4c(F)nn(C)c4c3)s2)OC1]

Into a 25 mL round-bottom flask were added tert-butyl (2S,7R)-2-{[(1S)-1-cyano-2-[5-(3-fluoro-1-methylindazol-6-yl)-1,3-thiazol-2-yl]ethyl]carbamoyl}-7-methoxy-1,4-oxazocane-4-carboxylate (140 mg, 0.24 mmol, 1.0 equiv), ACN (4 mL) and TsOH·H₂O (139 mg, 0.73 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A36 (17.1 mg, 15%) as a white solid. LCMS (ES, m/z): [M+H]⁺: 473.0. ¹H NMR (400 MHz, DMSO-d₆) δ 8.81 (d, J=8.6 Hz, 1H), 8.32 (s, 1H), 7.95 (s, 1H), 7.77 (d, J=8.5 Hz, 1H), 7.48 (d, J=8.4 Hz, 1H), 5.25 (q, J=7.8 Hz, 1H), 4.13 (dd, J=11.4, 4.3 Hz, 1H), 3.99-3.96 (m, 4H), 3.75-3.55 (m, 3H), 3.40-3.34 (m, 1H), 3.26 (s, 3H), 3.09 (dd, J=14.4, 3.1 Hz, 1H), 3.05-2.97 (m, 1H), 2.50-2.41 (m, 2H), 1.97-1.87 (m, 1H), 1.64-1.55 (m, 1H).

### Example 28: Synthesis of (2S,7R)—N—((S)-1-cyano-2-(5-(5,6,7,8-tetrahydro-1,5-naphthyridin-2-yl)thiazol-2-yl)ethyl)-7-methoxy-1,4-oxazocane-2-carboxamide (Compound A37)

### Step 1. Synthesis of tert-butyl (S)-(1-cyano-2-(5-(4,4,5,5-tetramethyl-1,3,2-dioxaborolan-2-yl)thiazol-2-yl)ethyl)carbamate

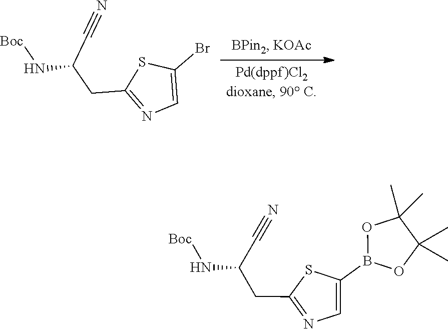 [SMILES: CC(C)(C)OC(=O)N[C@H](C#N)Cc1ncc(Br)s1 ; CC(C)(C)OC(=O)N[C@H](C#N)Cc1ncc(B2OC(C)(C)C(C)(C)O2)s1]

To a solution of tert-butyl N-[(1S)-2-(5-bromo-1,3-thiazol-2-yl)-1-cyanoethyl]carbamate (200 mg, 0.60 mmol, 1.0 equiv), bis(pinacolato)diboron (184 mg, 0.72 mmol, 1.2 equiv) in dioxane (2 mL) was treated with KOAc (118 mg, 1.20 mmol, 2.0 equiv) and Pd(dppf)Cl₂ (44 mg, 0.06 mmol, 0.1 equiv) at room temperature under nitrogen atmosphere. The resulting mixture was stirred for 2 h at 90° C. under nitrogen atmosphere. The reaction was cooled to room temperature, the precipitated solid was filtered by filtration and washed with dioxane (2 mL). The filtrate was concentrated under vacuum. The crude product was used in the next step directly without further purification. LCMS (ES) [M+1]⁺ m/z: 380.

### Step 2. Synthesis of tert-butyl (S)-(1-cyano-2-(5-(5,6,7,8-tetrahydro-1,5-naphthyridin-2-yl)thiazol-2-yl)ethyl)carbamate

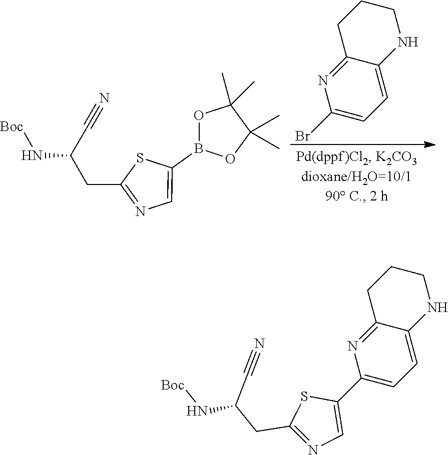

To a solution of tert-butyl N-[(1S)-1-cyano-2-[5-(4,4,5,5-tetramethyl-1,3,2-dioxaborolan-2-yl)-1,3-thiazol-2-yl]ethyl]carbamate (330 mg, 0.87 mmol, 1.0 equiv, crude of last step), 2-bromo-5,6,7,8-tetrahydro-1,5-naphthyridine (111 mg, 0.52 mmol, 0.6 equiv), in dioxane (5 mL), H₂O (0.5 mL) were added K₂CO₃ (241 mg, 1.74 mmol, 2.0 equiv) and Pd(dppf)Cl₂·CH₂Cl₂ (70.88 mg, 0.09 mmol, 0.1 equiv) in sequence. The mixture was stirred for 2 h at 90° C. under nitrogen atmosphere. The reaction was cooled to room temperature, concentrated under vacuum. The residue was purified by silica gel column chromatography, eluted with PE/EA (1:2) to afford tert-butyl N-[(1S)-1-cyano-2-[5-(5,6,7,8-tetrahydro-1,5-naphthyridin-2-yl)-1,3-thiazol-2-yl]ethyl]carbamate (130 mg, 39%) as a yellow oil. LCMS (ES) [M+1]⁺ m/z: 386.

### Step 3. Synthesis of (S)-2-amino-3-(5-(5,6,7,8-tetrahydro-1,5-naphthyridin-2-yl)thiazol-2-yl)propanenitrile

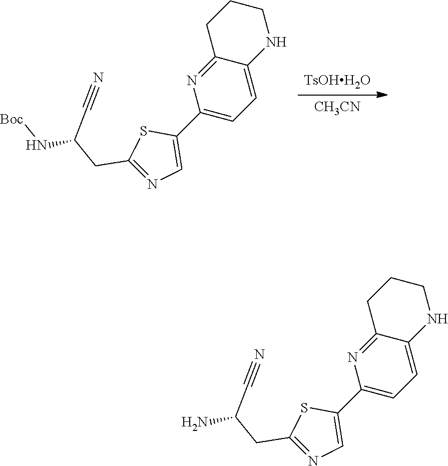

A solution of tert-butyl N-[(1S)-1-cyano-2-[5-(5,6,7,8-tetrahydro-1,5-naphthyridin-2-yl)-1,3-thiazol-2-yl]ethyl]carbamate (120 mg, 0.31 mmol, 1.0 equiv) and TsOH·H₂O (177 mg, 0.93 mmol, 3.0 equiv) in CH₃CN (2 mL) was stirred for 2 h at room temperature. The mixture was basified to pH 8 with saturated NaHCO₃ (aq.). The aqueous layer was extracted with EtOAc (20 mL×2). The resulting mixture was dried over anhydrous sodium sulfate. After filtration, the filtrate was concentrated under vacuum. The residue was purified by silica gel column chromatography, eluted with PE/EA to afford (2S)-2-amino-3-[5-(5,6,7,8-tetrahydro-1,5-naphthyridin-2-yl)-1,3-thiazol-2-yl]propanenitrile (70 mg, 79%) as a yellow solid. LCMS (ES) [M+1]⁺ m/z: 286.

### Step 4. Synthesis of tert-butyl (2S,7R)-2-(((S)-1-cyano-2-(5-(5,6,7,8-tetrahydro-1,5-naphthyridin-2-yl)thiazol-2-yl)ethyl)carbamoyl)-7-methoxy-1,4-oxazocane-4-carboxylate

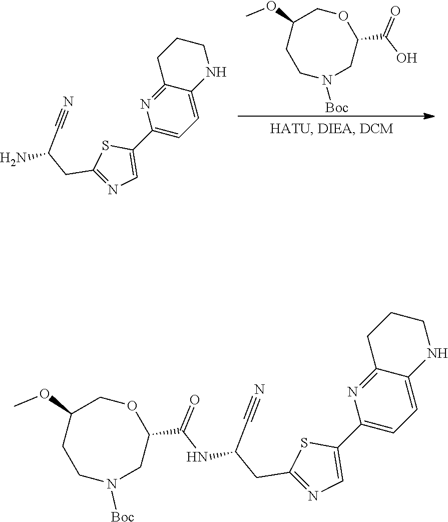

Into a 8 mL vial were added (2S)-2-amino-3-[5-(5,6,7,8-tetrahydro-1,5-naphthyridin-2-yl)-1,3-thiazol-2-yl]propanenitrile (70 mg, 0.25 mmol, 1.0 equiv), (2S,7R)-4-(tert-butoxycarbonyl)-7-methoxy-1,4-oxazocane-2-carboxylic acid (78 mg, 0.27 mmol, 1.1 equiv), DCM (2 mL), DIEA (95 mg, 0.74 mmol, 3.0 equiv). To the mixture, HATU (112 mg, 0.29 mmol, 1.2 equiv) was added at 0° C. The resulting mixture was stirred for 2 h at 0° C. Concentrated to remove the solvent, the residue was purified by silica gel column chromatography, eluted with PE/EA (1:1) to afford tert-butyl (2S,7R)-2-{[(1S)-1-cyano-2-[5-(5,6,7,8-tetrahydro-1,5-naphthyridin-2-yl)-1,3-thiazol-2-yl]ethyl]carbamoyl}-7-methoxy-1,4-oxazocane-4-carboxylate (90 mg, 66%) as a yellow oil. LCMS (ES) [M+1]⁺ m/z: 557.

### Step 5. Synthesis of (2S,7R)—N—((S)-1-cyano-2-(5-(5,6,7,8-tetrahydro-1,5-naphthyridin-2-yl)thiazol-2-yl)ethyl)-7-methoxy-1,4-oxazocane-2-carboxamide

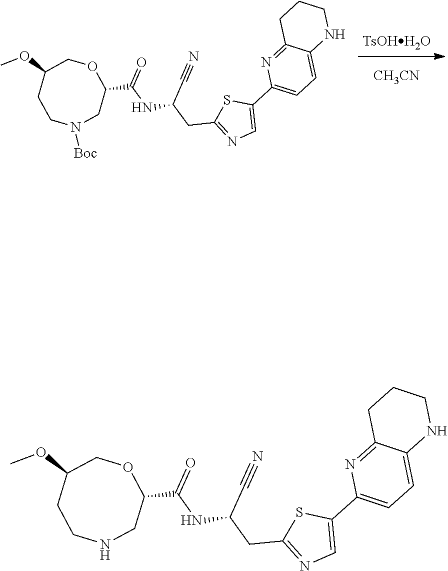

Into a 8 mL vial were added tert-butyl (2S,7R)-2-{[(1S)-1-cyano-2-[5-(5,6,7,8-tetrahydro-1,5-naphthyridin-2-yl)-1,3-thiazol-2-yl]ethyl]carbamoyl}-7-methoxy-1,4-oxazocane-4-carboxylate (80 mg, 0.14 mmol, 1.0 equiv), CH₃CN (3 mL) and TsOH·H₂O (82 mg, 0.43 mmol, 3.0 equiv) at room temperature. The resulting mixture was stirred for 3 h at room temperature. The reaction solution was purified by reverse phase flash with the following conditions: mobile phase, MeCN in Water (0.1% NH₃·H₂O), 10% to 70% gradient in 10 min. This resulted in Compound A37 (15.7 mg, 24%) as a yellow solid. LCMS (ES) [M+1]⁺ m/z: 457.1. ¹H NMR (300 MHz, DMSO-d₆) δ 8.76 (d, J=8.5 Hz, 1H), 8.00 (d, J=3.1 Hz, 1H), 7.43 (d, J=8.4 Hz, 1H), 6.79 (d, J=8.4 Hz, 1H), 6.16 (s, 1H), 5.15 (q, J=7.7 Hz, 1H), 4.10 (dd, J=11.5, 4.1 Hz, 1H), 3.93 (dd, J=9.0, 3.0 Hz, 1H), 3.65-3.40 (m, 3H), 3.39-3.30 (m, 4H), 3.28-3.13 (m, 2H), 3.11-2.92 (m, 2H), 2.75 (t, J=6.5 Hz, 2H), 2.48-2.37 (m, 2H), 1.86 (t, J=6.1 Hz, 3H), 1.56 (dt, J=15.5, 8.7 Hz, 1H).

### Example 29: Synthesis of (2S,7R)—N-{1-cyano-2-[2-(3-fluoro-1-methylindazol-6-yl)-1,3-thiazol-5-yl]ethyl}-7-methoxy-1,4-oxazocane-2-carboxamide (Compound A38)

### Step 1. Synthesis of tert-butyl (2S,7R)-2-{[(1S)-1-cyano-2-[2-(3-fluoro-1-methylindazol-6-yl)-1,3-thiazol-5-yl]ethyl]carbamoyl}-7-methoxy-1,4-oxazocane-4-carboxylate

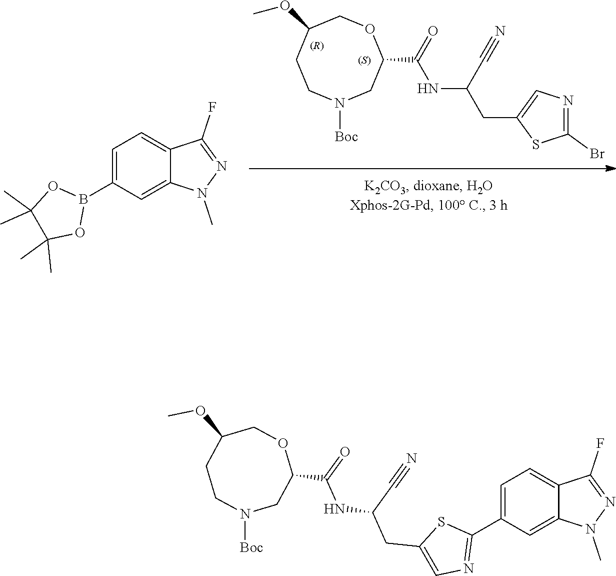

To a solution of 3-fluoro-1-methyl-6-(4,4,5,5-tetramethyl-1,3,2-dioxaborolan-2-yl)indazole (110 mg, 0.39 mmol, 1.0 equiv) and tert-butyl (2S,7R)-2-{[2-(2-bromo-1,3-thiazol-5-yl)-1-cyanoethyl]carbamoyl}-7-methoxy-1,4-oxazocane-4-carboxylate (200 mg, 0.39 mmol, 1.0 equiv) in 1,4-dioxane (5 mL) and H₂O (0.5 mL) was added XPhos Pd G2 (31 mg, 0.04 mmol, 0.1 equiv) and K₂CO₃ (110 mg, 0.79 mmol, 2.0 equiv). The mixture was stirred at 100° C. for 3 h under nitrogen atmosphere. The reaction was cooled to room temperature, concentrated to remove the solvent. The residue was purified by silica gel column chromatography, eluted with PE/THF (1:1) to afford tert-butyl (2S,7R)-2-{[(1S)-1-cyano-2-[2-(3-fluoro-1-methylindazol-6-yl)-1,3-thiazol-5-yl]ethyl]carbamoyl}-7-methoxy-1,4-oxazocane-4-carboxylate (100 mg, 43.8% yield, 95% purity) as a colorless oil. LCMS (ES, m/z): [M+H]⁺: 573.

### Step 2. Synthesis of (2S,7R)—N-{1-cyano-2-[2-(3-fluoro-1-methylindazol-6-yl)-1,3-thiazol-5-yl]ethyl}-7-methoxy-1,4-oxazocane-2-carboxamide

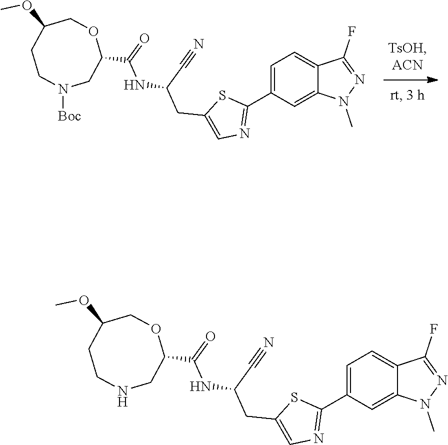 [SMILES: CO[C@@H]1CCN(C(=O)OC(C)(C)C)C[C@@H](C(=O)N[C@H](C#N)Cc2cnc(-c3ccc4c(F)nn(C)c4c3)s2)OC1 ; CO[C@@H]1CCNC[C@@H](C(=O)N[C@H](C#N)Cc2cnc(-c3ccc4c(F)nn(C)c4c3)s2)OC1]

Into a 25 mL round-bottom flask were added tert-butyl (2S,7R)-2-({1-cyano-2-[2-(3-fluoro-1-methylindazol-6-yl)-1,3-thiazol-5-yl]ethyl}carbamoyl)-7-methoxy-1,4-oxazocane-4-carboxylate (100 mg, 0.17 mmol, 1.0 equiv), acetonitrile (3 mL) and TsOH (90 mg, 0.52 mmol, 3.0 equiv) at room temperature. The resulting mixture was stirred at room temperature for 3 h. The reaction solution was purified by reversed-phase flash chromatography with the following conditions: column, C18 silica gel-120 g; mobile phase, MeCN in Water (0.1% NH₃·H₂O), 10% to 50% gradient in 15 min; detector, UV 254 nm. This resulted in Compound A38 (25 mg, 30%) as a white solid. LCMS (ES, m/z): [M+H]⁺: 473.1. ¹H NMR (400 MHz, DMSO-d₆) δ 8.81 (dd, J=11.8, 8.6 Hz, 1H), 8.19-8.13 (m, 1H), 7.86-7.78 (m, 2H), 7.74-7.69 (m, 1H), 5.10-4.99 (m, 1H), 4.20-4.10 (m, 1H), 4.00 (s, 3H), 3.98-3.90 (m, 1H), 3.67-3.45 (m, 3H), 3.40-3.35 (m, 1H), 3.26 (s, 3H), 3.18-2.93 (m, 2H), 2.60-2.52 (m, 2H), 1.97-1.91 (m, 1H), 1.64-1.54 (m, 1H).

### Example 30: Synthesis of (2S,7R)—N-{1-cyano-2-[2-(5,6,7,8-tetrahydro-1,5-naphthyridin-2-yl)-1,3-thiazol-5-yl]ethyl}-7-methoxy-1,4-oxazocane-2-carboxamide (Compound A39)

### Step 1. Synthesis of 2-(trimethylstannyl)-5,6,7,8-tetrahydro-1,5-naphthyridine

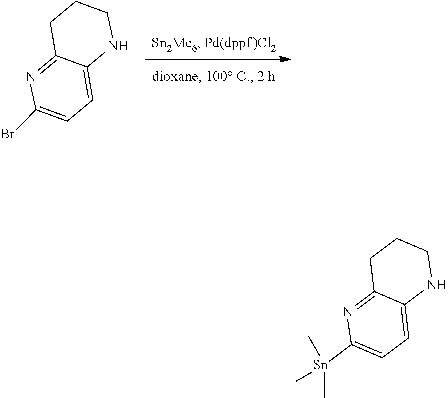

To a solution of 2-bromo-5,6,7,8-tetrahydro-1,5-naphthyridine (800 mg, 3.75 mmol, 1.00 equiv) and Sn₂Me₆ (1.23 g, 3.75 mmol, 1.00 equiv) in dioxane (10 mL) was added Pd(dppf)Cl₂ (274 mg, 0.37 mmol, 0.10 equiv) at room temperature. The resulting mixture was stirred for 2 h at 100° C. under nitrogen atmosphere. The residue was filtered and concentrated to give 2-(trimethylstannyl)-5,6,7,8-tetrahydro-1,5-naphthyridine (950 mg, 85.20%) as a yellow oil, which was used for next step without further purification. LCMS (ES, m/z): [M+H]⁺: 299.

### Step 2. Synthesis of 2-[(diphenylmethylidene)amino]-3-[2-(5,6,7,8-tetrahydro-1,5-naphthyridin-2-yl)-1,3-thiazol-5-yl]propanenitrile

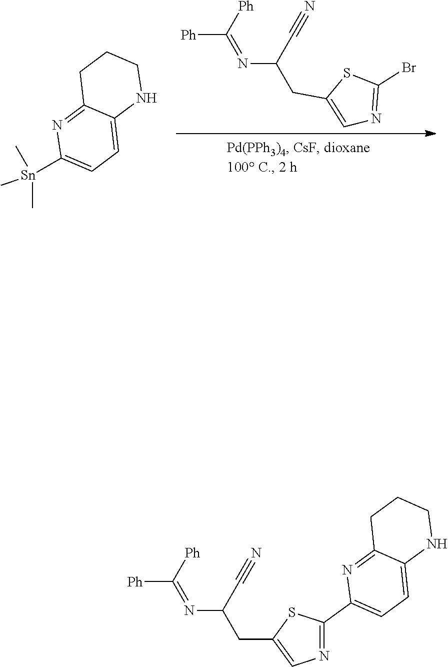 [SMILES: [CH3][Sn]([CH3])([CH3])[c]1ccc2c(n1)CCCN2 ; N#CC(Cc1cnc(Br)s1)N=C(c1ccccc1)c1ccccc1 ; N#CC(Cc1cnc(-c2ccc3c(n2)CCCN3)s1)N=C(c1ccccc1)c1ccccc1]

To a solution of 2-(trimethylstannyl)-5,6,7,8-tetrahydro-1,5-naphthyridine (950 mg, 3.19 mmol, 1.0 equiv), 3-(2-bromo-1,3-thiazol-5-yl)-2-[(diphenylmethylidene)amino]propanenitrile (1.01 g, 2.55 mmol, 0.80 equiv) and CsF (48 mg, 0.32 mmol, 0.10 equiv) in dioxane (12 mL) was added Pd(PPh₃)₄ (369 mg, 0.32 mmol, 0.10 equiv) at room temperature. The resulting mixture was stirred for 2 h at 100° C. under nitrogen atmosphere. The reaction was concentrated. The residue was purified by silica gel column chromatography, eluted with PE/THF (1:1) to afford 2-[(diphenylmethylidene)amino]-3-[2-(5,6,7,8-tetrahydro-1,5-naphthyridin-2-yl)-1,3-thiazol-5-yl]propanenitrile (600 mg, 41.72%) as a yellow oil. LCMS (ES, m/z): [M+H]⁺: 450.

### Step 3. Synthesis of 2-amino-3-[2-(5,6,7,8-tetrahydro-1,5-naphthyridin-2-yl)-1,3-thiazol-5-yl]propanenitrile

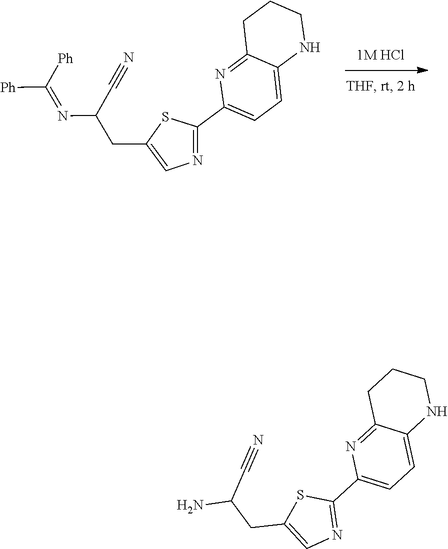

Into a 50 mL round-bottom flask were added 2-[(diphenylmethylidene)amino]-3-[2-(5,6,7,8-tetrahydro-1,5-naphthyridin-2-yl)-1,3-thiazol-5-yl]propanenitrile (700 mg, 1.55 mmol, 1.0 equiv) in THF (40 mL) and H₂O (4 mL) was added HCl (1M) (2 mL) at room temperature. The resulting mixture was stirred for additional 2 h at room temperature. The mixture was basified to Ph=10 with saturated Na₂CO₃ (aq.). The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/THF (1:1) to afford 2-amino-3-[2-(5,6,7,8-tetrahydro-1,5-naphthyridin-2-yl)-1,3-thiazol-5-yl]propanenitrile (200 mg, 45.01%) as a white semi-solid. LCMS (ES, m/z): [M+H]⁺: 286.

### Step 4. Synthesis of tert-butyl (2S,7R)-2-({1-cyano-2-[2-(5,6,7,8-tetrahydro-1,5-naphthyridin-2-yl)-1,3-thiazol-5-yl]ethyl}carbamoyl)-7-methoxy-1,4-oxazocane-4-carboxylate

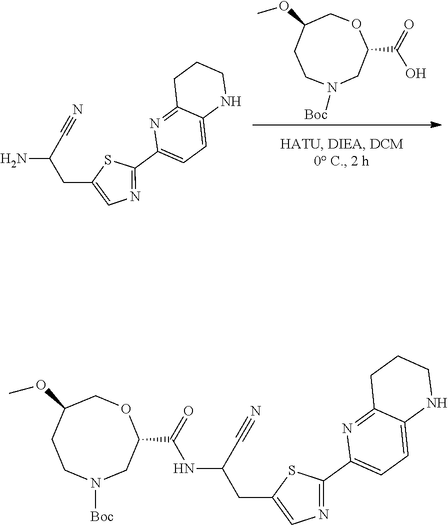 [SMILES: N#CC(N)Cc1cnc(-c2ccc3c(n2)CCCN3)s1 ; CO[C@@H]1CCN(C(=O)OC(C)(C)C)C[C@@H](C(=O)O)OC1 ; CO[C@@H]1CCN(C(=O)OC(C)(C)C)C[C@@H](C(=O)NC(C#N)Cc2cnc(-c3ccc4c(n3)CCCN4)s2)OC1]

To a solution of 2-amino-3-[2-(5,6,7,8-tetrahydro-1,5-naphthyridin-2-yl)-1,3-thiazol-5-yl]propanenitrile (120 mg, 0.42 mmol, 1.0 equiv), (2S,7R)-4-(tert-butoxycarbonyl)-7-methoxy-1,4-oxazocane-2-carboxylic acid (121 mg, 0.42 mmol, 1.0 equiv) and DIEA (163 mg, 1.26 mmol, 3.00 equiv) in DCM (3 mL) was added HATU (239 mg, 0.63 mmol, 1.5 equiv) at 0° C. The resulting mixture was stirred for 2 h at 0° C. The reaction was concentrated. The residue was purified by silica gel column chromatography, eluted with PE/THF (1:1) to afford tert-butyl (2S,7R)-2-({1-cyano-2-[2-(5,6,7,8-tetrahydro-1,5-naphthyridin-2-yl)-1,3-thiazol-5-yl]ethyl}carbamoyl)-7-methoxy-1,4-oxazocane-4-carboxylate (120 mg, 51.26%) as a white semi-solid. LCMS (ES, m/z): [M+H]⁺: 557.

### Step 5. Synthesis of (2S,7R)—N-{1-cyano-2-[2-(5,6,7,8-tetrahydro-1,5-naphthyridin-2-yl)-1,3-thiazol-5-yl]ethyl}-7-methoxy-1,4-oxazocane-2-carboxamide

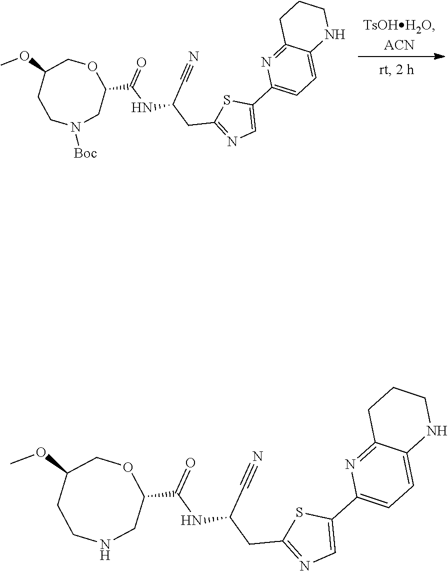

Into a 8 mL vial were added tert-butyl (2S,7R)-2-({1-cyano-2-[2-(5,6,7,8-tetrahydro-1,5-naphthyridin-2-yl)-1,3-thiazol-5-yl]ethyl}carbamoyl)-7-methoxy-1,4-oxazocane-4-carboxylate (120 mg, 0.21 mmol, 1.00 equiv), TsOH·H₂O (111 mg, 0.64 mmol, 3.00 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 80% gradient in 10 min; detector, UV 254 nm. This resulted in Compound A39 (25 mg, 25.5% yield) as a white solid. LCMS (ES, m/z): [M+H]⁺: 457.5. ¹H NMR (400 MHz, DMSO-d₆) δ 8.74 (dd, J=8.6, 4.1 Hz, 1H), 7.60 (dd, J=8.5, 1.9 Hz, 1H), 7.55 (d, J=1.8 Hz, 1H), 6.83 (dd, J=8.4, 1.5 Hz, 1H), 6.45 (d, J=3.1 Hz, 1H), 5.01-4.88 (m, 1H), 4.21-4.08 (m, 1H), 3.97-3.85 (m, 1H), 3.67-3.52 (m, 1H), 3.47-3.34 (m, 3H), 3.27-3.20 (m, 5H), 3.15-2.96 (m, 2H), 2.80 (t, J=6.3 Hz, 2H), 2.60-2.53 (m, 1H), 2.47-2.40 (m, 1H), 1.99-1.86 (m, 3H), 1.65-1.53 (m, 1H).

### Example 31: Synthesis of (2S)—N-[(1S)-1-cyano-2-[4-(5-methyl-7,8-dihydro-6H-1,5-naphthyridin-3-yl)phenyl]ethyl]-1,4-oxazepane-2-carboxamide (Compound A40)

### Step 1. Synthesis of 7-bromo-1-methyl-3,4-dihydro-2H-1,5-naphthyridine

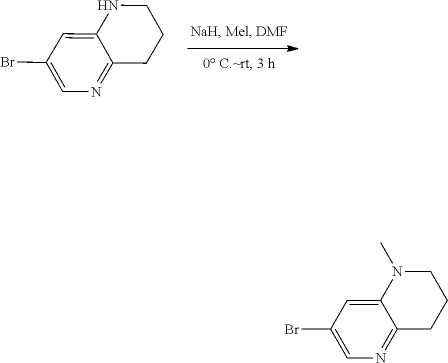

A solution of 3-bromo-5,6,7,8-tetrahydro-1,5-naphthyridine (90 mg, 0.42 mmol, 1.0 equiv) in DMF (5 mL) was treated with NaH (20 mg, 0.50 mmol, 1.2 equiv, 60%) at 0° C. under nitrogen atmosphere and stirred for 30 min. This was followed by the addition of MeI (72 mg, 0.51 mmol, 1.2 equiv) dropwise at room temperature. The resulting mixture was stirred for additional 3 h at room temperature. The reaction was quenched with water (15 mL), extracted with EtOAc (3×20 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (2:1) to afford 7-bromo-1-methyl-3,4-dihydro-2H-1,5-naphthyridine (40 mg, 42%) as a colorless oil. LCMS (ES, m/z): [M+H]⁺: 227.

### Step 2. Synthesis of (2S)-2-{[(1S)-1-cyano-2-[4-(5-methyl-7,8-dihydro-6H-1,5-naphthyridin-3-yl)phenyl]ethyl]carbamoyl}-1,4-oxazepane-4-carboxylate

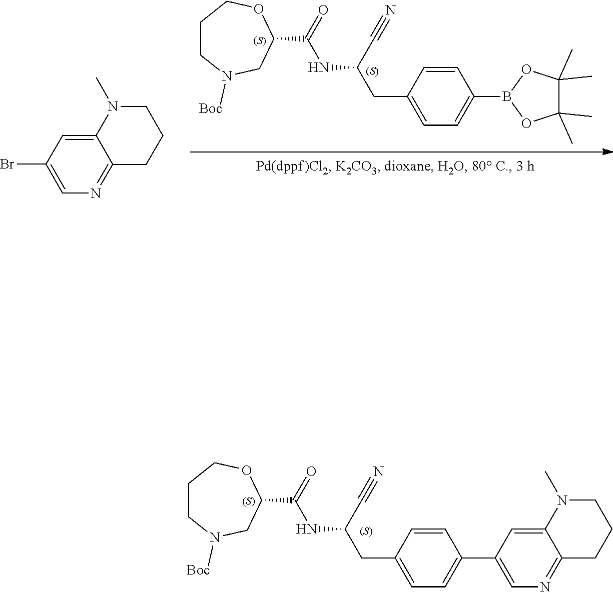

To a solution of 7-bromo-1-methyl-3,4-dihydro-2H-1,5-naphthyridine (40 mg, 0.17 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (105 mg, 0.21 mmol, 1.2 equiv) in 1,4-dioxane (5 mL), H₂O (0.5 mL) were added Pd(dppf)Cl₂ (12 mg, 0.01 mmol, 0.1 equiv) and K₂CO₃ (49 mg, 0.35 mmol, 2.0 equiv). The mixture was stirred for 3 h at 80° C. under nitrogen atmosphere. The reaction was cooled to room temperature, concentrated under reduced pressure. The residue was purified by silica gel column chromatography, eluted with PE/THF (1:1) to afford tert-butyl (2S)-2-{[(1S)-1-cyano-2-[4-(5-methyl-7,8-dihydro-6H-1,5-naphthyridin-3-yl)phenyl]ethyl]carbamoyl}-1,4-oxazepane-4-carboxylate (80 mg, 87.4%) as a foam solid. LCMS (ES, m/z): [M+H]⁺: 520.

### Step 3. Synthesis of (2S)—N-[(1S)-1-cyano-2-[4-(5-methyl-7,8-dihydro-6H-1,5-naphthyridin-3-yl)phenyl]ethyl]-1,4-oxazepane-2-carboxamide

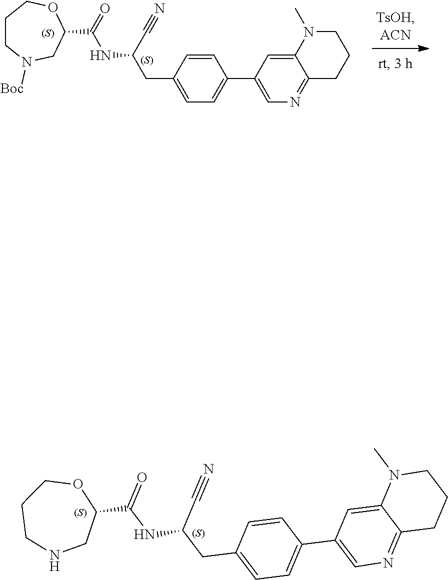

Into a 25 mL round-bottom flask were added tert-butyl (2S)-2-{[(1S)-1-cyano-2-[4-(5-methyl-7,8-dihydro-6H-1,5-naphthyridin-3-yl)phenyl]ethyl]carbamoyl}-1,4-oxazepane-4-carboxylate (80 mg, 0.15 mmol, 1.0 equiv), ACN (3 mL) and TsOH·H₂O (87 mg, 0.46 mmol, 3.0 equiv) at room temperature. The mixture was stirred for 3 h. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A40 (18.6 mg, 28.8%) as a white solid. LCMS (ES, m/z): [M+H]⁺: 420.1. ¹H NMR (400 MHz, DMSO-d₆) δ 8.60 (d, J=8.5 Hz, 1H), 7.96 (d, J=1.9 Hz, 1H), 7.62 (d, J=7.9 Hz, 2H), 7.38 (d, J=7.9 Hz, 2H), 7.04 (d, J=1.9 Hz, 1H), 5.02 (q, J=8.2 Hz, 1H), 4.01 (dd, J=8.0, 3.6 Hz, 1H), 3.88-3.81 (m, 1H), 3.76-3.69 (m, 1H), 3.26-3.15 (m, 4H), 3.05 (dd, J=14.2, 3.7 Hz, 1H), 2.92 (s, 3H), 2.85-2.75 (m, 3H), 2.68-2.55 (m, 2H), 2.03-1.96 (m, 2H), 1.82-1.64 (m, 2H).

### Example 32: Synthesis of (2S)—N-[(1S)-1-cyano-2-[4-(5,6,7,8-tetrahydro-1,5-naphthyridin-3-yl)phenyl]ethyl]-1,4-oxazepane-2-carboxamide (Compound A41)

### Step 1. Synthesis of tert-butyl (2S)-2-{[(1S)-1-cyano-2-[4-(5,6,7,8-tetrahydro-1,5-naphthyridin-3-yl)phenyl]ethyl]carbamoyl}-1,4-oxazepane-4-carboxylate

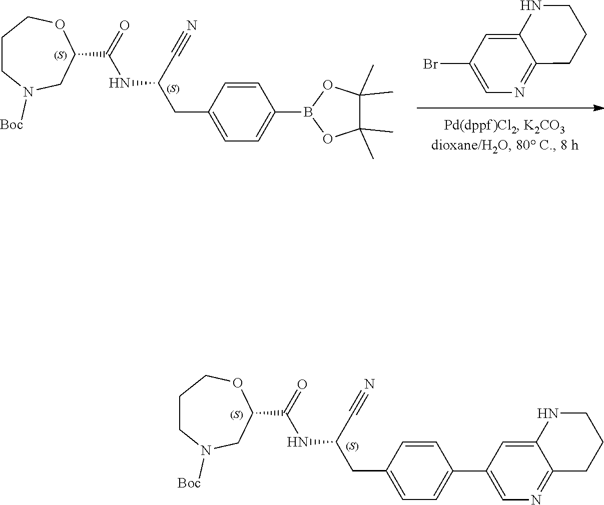

To a solution of 3-bromo-5,6,7,8-tetrahydro-1,5-naphthyridine (80 mg, 0.37 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (225 mg, 0.45 mmol, 1.2 equiv) in 1,4-dioxane (5 mL) and H₂O (0.5 mL), K₂CO₃ (104 mg, 0.75 mmol, 2.0 equiv), Pd(dppf)Cl₂ (27 mg, 0.03 mmol, 0.1 equiv) were added in sequence. The mixture was stirred for 8 h at 80° C. under nitrogen atmosphere. The reaction was cooled to room temperature, concentrated to remove the solvent, the residue was purified by silica gel column chromatography, eluted with PE/THF (1:1.5) to afford tert-butyl (2S)-2-{[(1S)-1-cyano-2-[4-(5,6,7,8-tetrahydro-1,5-naphthyridin-3-yl)phenyl]ethyl]carbamoyl}-1,4-oxazepane-4-carboxylate (140 mg, 73.7%) as white semi-solid. LCMS (ES, m/z): [M+H]⁺: 506.

### Step 2. Synthesis of (2S)—N-[(1S)-1-cyano-2-[4-(5,6,7,8-tetrahydro-1,5-naphthyridin-3-yl)phenyl]ethyl]-1,4-oxazepane-2-carboxamide

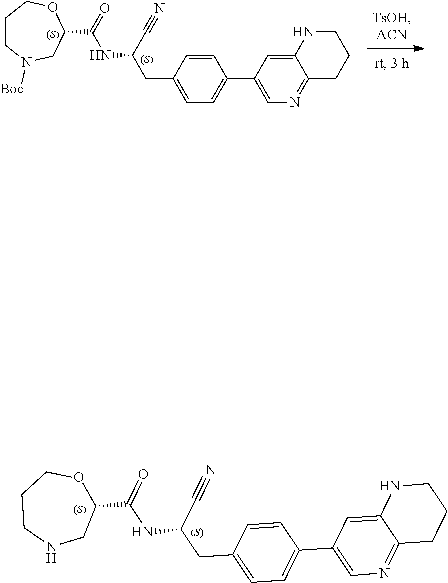

Into a 25 mL round-bottom flask were added tert-butyl (2S)-2-{[(1S)-1-cyano-2-[4-(5,6,7,8-tetrahydro-1,5-naphthyridin-3-yl)phenyl]ethyl]carbamoyl}-1,4-oxazepane-4-carboxylate (100 mg, 0.19 mmol, 1.0 equiv) and TsOH·H₂O (113 mg, 0.59 mmol, 3.0 equiv) at room temperature. The resulting mixture was stirred for 3 hat room temperature. The reaction solution was purified by reversed-phase flash chromatography with the following conditions: column, C18 silica gel-120 g; mobile phase, MeCN in Water (0.1% NH₃·H₂O), 10% to 80% gradient in 15 min; detector, UV 254 nm. This resulted in Compound A41 (16.5 mg, 20.6%) as white solid. LCMS (ES, m/z): [M+H]⁺: 406.1. ¹H NMR (400 MHz, DMSO-d₆) δ 8.60 (d, J=8.5 Hz, 1H), 7.90 (d, J=2.0 Hz, 1H), 7.51 (d, J=7.9 Hz, 2H), 7.36 (d, J=8.0 Hz, 2H), 6.96 (d, J=2.0 Hz, 1H), 5.91 (d, J=2.9 Hz, 1H), 5.02 (q, J=8.3 Hz, 1H), 3.99 (dd, J=7.8, 3.7 Hz, 1H), 3.87-3.80 (m, 1H), 3.75-3.69 (m, 1H), 3.26-3.12 (m, 4H), 3.02 (dd, J=14.3, 3.8 Hz, 1H), 2.81-2.72 (m, 3H), 2.63-2.55 (m, 2H), 1.93-1.87 (m, 2H), 1.76-1.67 (m, 2H).

### Example 33: Synthesis of (2S,6S)—N-[(1S)-1-cyano-2-(4-{1,3-dimethylpyrrolo[1,2-a]pyrazin-7-yl}phenyl)ethyl]-6-methoxy-1,4-oxazocane-2-carboxamide (Compound A42)

### Step 1. Synthesis of tert-butyl (2S,6S)-2-{[(1S)-1-cyano-2-(4-{1,3-dimethylpyrrolo[1,2-a]pyrazin-7-yl}phenyl)ethyl]carbamoyl}-6-methoxy-1,4-oxazocane-4-carboxylate

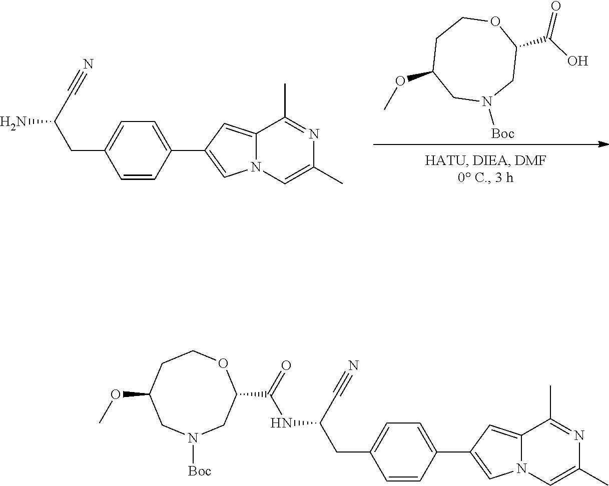

To a stirred solution of (2S,6S)-4-(tert-butoxycarbonyl)-6-methoxy-1,4-oxazocane-2-carboxylic acid (70 mg, 0.24 mmol, 1.0 equiv) and (2S)-2-amino-3-(4-{1,3-dimethylpyrrolo[1,2-a]pyrazin-7-yl}phenyl)propanenitrile (70 mg, 0.24 mmol, 1.0 equiv), DIEA (93 mg, 0.72 mmol, 3.0 equiv) in DMF (3 mL) was added HATU (110 mg, 0.29 mmol, 1.2 equiv) in portions at room temperature. The resulting mixture was stirred at 0° C. for 3 h. The resulting mixture was diluted with water (10 mL), extracted with EtOAc (3×30 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (1:1) to afford tert-butyl (2S,6S)-2-{[(1S)-1-cyano-2-(4-{1,3-dimethylpyrrolo[1,2-a]pyrazin-7-yl}phenyl)ethyl]carbamoyl}-6-methoxy-1,4-oxazocane-4-carboxylate (70 mg, 51%) as a colorless semi-solid. LCMS (ES, m/z): [M+H]⁺: 562.

### Step 2. Synthesis of (2S,6S)—N-[(1S)-1-cyano-2-(4-{1,3-dimethylpyrrolo[1,2-a]pyrazin-7-yl}phenyl)ethyl]-6-methoxy-1,4-oxazocane-2-carboxamide

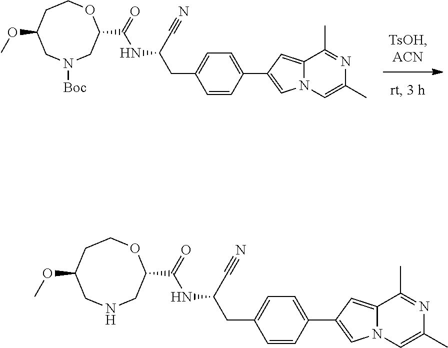

Into a 25 mL round-bottom flask were added tert-butyl (2S,6S)-2-{[(1S)-1-cyano-2-(4-{1,3-dimethylpyrrolo[1,2-a]pyrazin-7-yl}phenyl)ethyl]carbamoyl}-6-methoxy-1,4-oxazocane-4-carboxylate (70 mg, 0.12 mmol, 1.0 equiv) in ACN (3 mL) and TsOH·H₂O (71 mg, 0.37 mmol, 3.0 equiv) at room temperature. The resulting mixture was stirred at room temperature for 3 h. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A42 (13.4 mg, 23.3%) as a white solid. LCMS (ES, m/z): [M+H]f: 462.1. ¹H NMR (400 MHz, DMSO-d₆) δ 8.61 (d, J=8.5 Hz, 1H), 8.03 (d, J=1.6 Hz, 1H), 7.91 (s, 1H), 7.70 (d, J=8.1 Hz, 2H), 7.30 (d, J=8.0 Hz, 2H), 7.18 (d, J=1.7 Hz, 1H), 4.97 (q, J=8.3 Hz, 1H), 3.91 (dd, J=10.1, 2.5 Hz, 1H), 3.87-3.77 (m, 1H), 3.73-3.65 (m, 1H), 3.27-3.19 (m, 1H), 3.20 (s, 3H), 3.17-3.10 (m, 3H), 2.98 (dd, J=13.7, 2.4 Hz, 1H), 2.71-2.65 (m, 1H), 2.57 (s, 3H), 2.28 (s, 3H), 2.21-2.06 (m, 2H), 1.68-1.59 (m, 1H).

### Example 34: Synthesis of (2S,6S)—N—((S)-1-cyano-2-(4-(5,6,7,8-tetrahydro-1,5-naphthyridin-2-yl)phenyl)ethyl)-6-methoxy-1,4-oxazocane-2-carboxamide (Compound A43)

### Step 1. Synthesis of tert-butyl (2S,6S)-2-(((S)-1-cyano-2-(4-(5,6,7,8-tetrahydro-1,5-naphthyridin-2-yl)phenyl)ethyl)carbamoyl)-6-methoxy-1,4-oxazocane-4-carboxylate

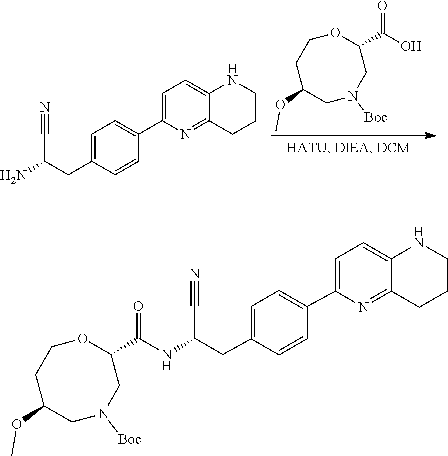

To a solution of (2S)-2-amino-3-[4-(5,6,7,8-tetrahydro-1,5-naphthyridin-2-yl)phenyl]propanenitrile (80 mg, 0.29 mmol, 1.0 equiv), (2S,6S)-4-(tert-butoxycarbonyl)-6-methoxy-1,4-oxazocane-2-carboxylic acid (91 mg, 0.31 mmol, 1.1 equiv), DIEA (112 mg, 0.86 mmol, 3.0 equiv) in DCM (3 mL) was treated with HATU (131 mg, 0.34 mmol, 1.2 equiv) at 0° C. The resulting mixture was stirred at 0° C. for 2 h. The reaction was quenched by the addition of water (10 mL). The aqueous layer was extracted with CH₂Cl₂ (15 mL×2). The resulting mixture was concentrated under vacuum. The residue was purified by silica gel column chromatography, eluted with PE/EA (1:3) to afford tert-butyl (2S,6S)-2-{[(1S)-1-cyano-2-[4-(5,6,7,8-tetrahydro-1,5-naphthyridin-2-yl)phenyl]ethyl]carbamoyl}-6-methoxy-1,4-oxazocane-4-carboxylate (120 mg, 76% yield) as a yellow solid. LCMS (ES) [M+1]⁺ m/z: 550.

### Step 2. Synthesis of (2S,6S)—N—((S)-1-cyano-2-(4-(5,6,7,8-tetrahydro-1,5-naphthyridin-2-yl)phenyl)ethyl)-6-methoxy-1,4-oxazocane-2-carboxamide

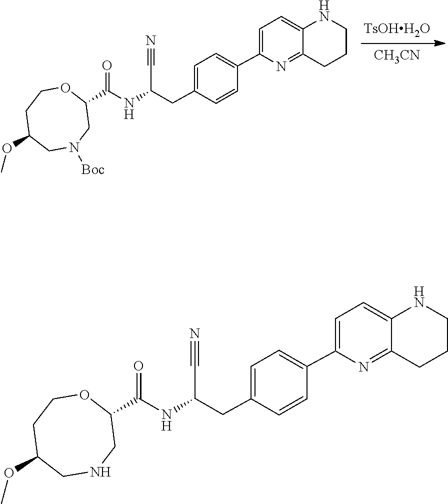

Into a 8 mL vial were added tert-butyl (2S,6S)-2-{[(1S)-1-cyano-2-[4-(5,6,7,8-tetrahydro-1,5-naphthyridin-2-yl)phenyl]ethyl]carbamoyl}-6-methoxy-1,4-oxazocane-4-carboxylate (90 mg, 0.16 mmol, 1.0 equiv), CH₃CN (3 mL) and TsOH·H₂O (93 mg, 0.49 mmol, 3.0 equiv) at room temperature. The resulting mixture was stirred at room temperature for 2 h. The reaction was purified by reverse phase flash with the following conditions: column C18 silica-120 g, mobile phase, MeCN in Water (0.1% NH₃·H₂O), 10% to 70% gradient in 10 min; detector, UV 254 nm. This resulted in Compound A43 (17 mg, 23%) as a white solid. LCMS (ES) [M+1]⁺ m/z: 450.2. ¹H NMR (300 MHz, DMSO-d₆) δ 8.60 (d, J=8.5 Hz, 1H), 7.83 (d, J=8.0 Hz, 2H), 7.46 (d, J=8.4 Hz, 1H), 7.26 (d, J=8.1 Hz, 2H), 6.83 (d, J=8.4 Hz, 1H), 6.00 (d, J=2.7 Hz, 1H), 4.95 (q, J=8.2 Hz, 1H), 3.90 (dd, J=10.1, 2.6 Hz, 1H), 3.82 (td, J=11.6, 2.9 Hz, 1H), 3.74-3.63 (m, 1H), 3.29-3.04 (m, 9H), 2.98 (dd, J=13.8, 2.6 Hz, 1H), 2.84 (t, J=6.4 Hz, 2H), 2.67 (dd, J=14.7, 3.7 Hz, 1H), 2.19 (brs, 1H), 2.18-2.08 (m, 2H), 1.92 (q, J=5.9 Hz, 2H), 1.62 (dd, J=14.8, 3.5 Hz, 1H).

### Example 35: Synthesis of (2S,6S)—N-[(1S)-1-cyano-2-(5-{1,3-dimethylpyrrolo[1,2-a]pyrazin-7-yl}thiophen-2-yl)ethyl]-6-methoxy-1,4-oxazocane-2-carboxamide (Compound A44)

### Step 1. Synthesis of tert-butyl (2S,6S)-2-{[(1S)-1-cyano-2-(5-{1,3-dimethylpyrrolo[1,2-a]pyrazin-7-yl}thiophen-2-yl)ethyl]carbamoyl}-6-methoxy-1,4-oxazocane-4-carboxylate

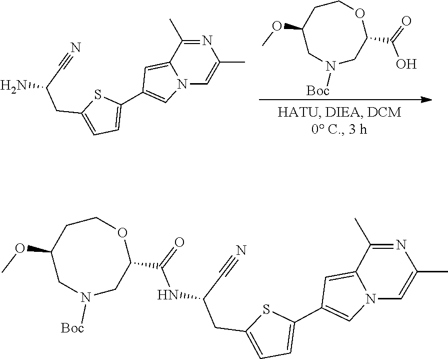

To a stirred solution of (2S)-2-amino-3-(5-{1,3-dimethylpyrrolo[1,2-a]pyrazin-7-yl}thiophen-2-yl)propanenitrile (72 mg, 0.24 mmol, 1.0 equiv) and (2S,6S)-4-(tert-butoxycarbonyl)-6-methoxy-1,4-oxazocane-2-carboxylic acid (70 mg, 0.24 mmol, 1.0 equiv), DIEA (94 mg, 0.72 mmol, 3.0 equiv) in DCM (3 mL) was added HATU (110 mg, 0.2 mmol, 1.2 equiv) in portions at 0° C. The resulting mixture was stirred at 0° C. for 3 h. Concentrated to remove the solvent, the residue was purified by silica gel column chromatography, eluted with PE/THF (1:1) to afford tert-butyl (2S,6S)-2-{[(1S)-1-cyano-2-(5-{1,3-dimethylpyrrolo[1,2-a]pyrazin-7-yl}thiophen-2-yl)ethyl]carbamoyl}-6-methoxy-1,4-oxazocane-4-carboxylate (120 mg, 87%) as a white semi-solid. LCMS (ES, m/z): [M+H]⁺: 568.

### Step 2. Synthesis of (2S,6S)—N-[(1S)-1-cyano-2-(5-{1,3-dimethylpyrrolo[1,2-a]pyrazin-7-yl}thiophen-2-yl)ethyl]-6-methoxy-1,4-oxazocane-2-carboxamide

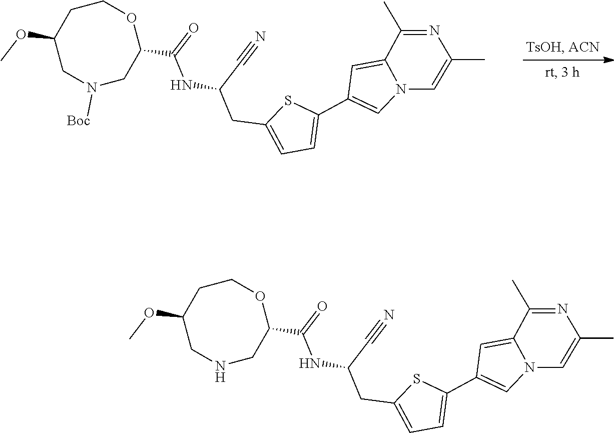

Into a 25 mL round-bottom flask were added tert-butyl (2S,6S)-2-{[(1S)-1-cyano-2-(5-{1,3-dimethylpyrrolo[1,2-a]pyrazin-7-yl}thiophen-2-yl)ethyl]carbamoyl}-6-methoxy-1,4-oxazocane-4-carboxylate (120 mg, 0.21 mmol, 1.0 equiv), ACN (3 mL) and TsOH·H₂O (120 mg, 0.63 mmol, 3.0 equiv) at room temperature. The resulting mixture was stirred at room temperature for 3 h. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A44 (17.6 mg, 17.8%) as a white solid. LCMS (ES, m/z): [M+H]⁺: 468.2. ¹H NMR (400 MHz, DMSO-d₆) δ 8.69 (d, J=8.6 Hz, 1H), 7.91 (s, 1H), 7.86 (d, J=1.6 Hz, 1H), 7.23 (d, J=3.6 Hz, 1H), 6.95 (t, J=1.3 Hz, 1H), 6.93 (d, J=3.6 Hz, 1H), 5.01-4.90 (m, 1H), 3.95 (dd, J=10.0, 2.6 Hz, 1H), 3.89-3.78 (m, 1H), 3.75-3.67 (m, 1H), 3.46-3.34 (m, 2H), 3.28-3.23 (m, 1H), 3.20 (s, 3H), 3.16 (d, J=14.3 Hz, 1H), 3.08-3.03 (m, 1H), 2.75-2.66 (m, 1H), 2.55 (s, 3H), 2.31-2.13 (m, 5H), 1.68-1.60 (m, 1H).

### Example 36: Synthesis of (2S,6S)—N-[(1S)-1-cyano-2-(5-{1,3-dimethylpyrrolo[1,2-a]pyrazin-7-yl}-1-benzothiophen-2-yl)ethyl]-6-methoxy-1,4-oxazocane-2-carboxamide (Compound A45) and (2S,6S)—N-[(1R)-1-cyano-2-(5-{1,3-dimethylpyrrolo[1,2-a]pyrazin-7-yl}-1-benzothiophen-2-yl)ethyl]-6-methoxy-1,4-oxazocane-2-carboxamide (Compound A46)

### Step 1. Synthesis of (2S,6S)-2-{[(1S)-1-cyano-2-(5-{1,3-dimethylpyrrolo[1,2-a]pyrazin-7-yl}-1-benzothiophen-2-yl)ethyl]carbamoyl}-6-methoxy-1,4-oxazocane-4-carboxylate

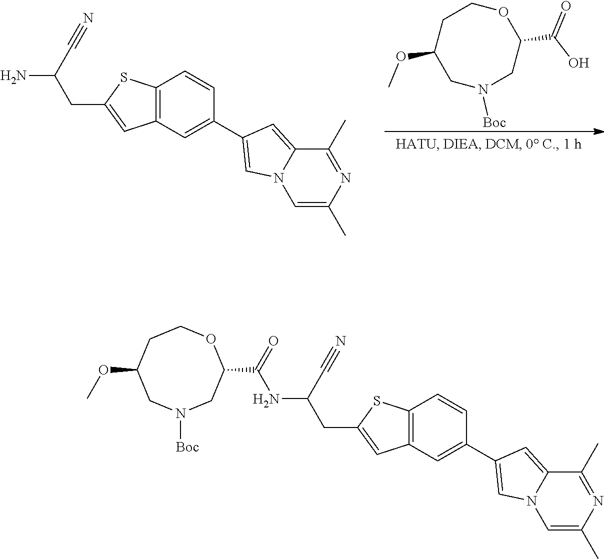

To a stirred solution of (2S,6S)-4-(tert-butoxycarbonyl)-6-methoxy-1,4-oxazocane-2-carboxylic acid (60 mg, 0.21 mmol, 1.0 equiv) and (2S)-2-amino-3-(5-{1,3-dimethylpyrrolo[1,2-a]pyrazin-7-yl}-1-benzothiophen-2-yl)propanenitrile (75 mg, 0.22 mmol, 1.05 equiv) in DCM (1 mL) were added DIEA (80 mg, 0.62 mmol, 3.0 equiv) and HATU (118 mg, 0.31 mmol, 1.5 equiv) in portions at 0° C. The resulting mixture was stirred at 0° C. for 1 h. The resulting mixture was concentrated under reduced pressure. The residue was purified by silica gel column chromatography, eluted with PE/THF (1:1) to afford tert-butyl (2S,6S)-2-{[(1S)-1-cyano-2-(5-{1,3-dimethylpyrrolo[1,2-a]pyrazin-7-yl}-1-benzothiophen-2-yl)ethyl]carbamoyl}-6-methoxy-1,4-oxazocane-4-carboxylate (100 mg, 78%) as a light yellow solid. LCMS (ES) [M+H]⁺ m/z: 618.

### Step 2. Synthesis of (2S,6S)—N-[(1S)-1-cyano-2-(5-{1,3-dimethylpyrrolo[1,2-a]pyrazin-7-yl}-1-benzothiophen-2-yl)ethyl]-6-methoxy-1,4-oxazocane-2-carboxamide

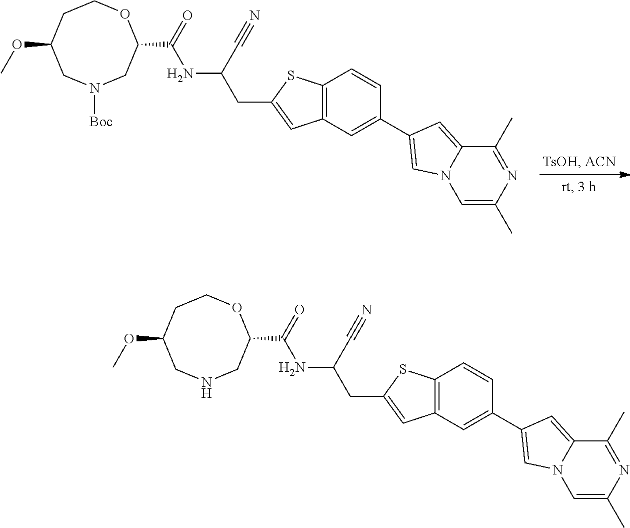

To a stirred solution of tert-butyl (2S,6S)-2-{[(1S)-1-cyano-2-(5-{1,3-dimethylpyrrolo[1,2-a]pyrazin-7-yl}-1-benzothiophen-2-yl)ethyl]carbamoyl}-6-methoxy-1,4-oxazocane-4-carboxylate (100 mg, 0.16 mmol, 1.0 equiv) in ACN (2 mL) was added TsOH (83 mg, 0.48 mmol, 3.0 equiv). The resulting mixture was stirred at room temperature for 3 h. The mixture was purified by reversed-phase flash chromatography with the following conditions: column, C18 silica gel-120 g; mobile phase, MeCN in Water (0.1% NH₃·H₂O), 10% to 60% gradient in 15 min; detector, UV 254 nm. This resulted in (2S,6S)—N-[(1S)-1-cyano-2-(5-{1,3-dimethylpyrrolo[1,2-a]pyrazin-7-yl}-1-benzothiophen-2-yl)ethyl]-6-methoxy-1,4-oxazocane-2-carboxamide (25 mg, 30%) as a white solid. LCMS (ES) [M+H]⁺ m/z: 518.

### Step 3. Synthesis of (2S,6S)—N-[(1S)-1-cyano-2-(5-{1,3-dimethylpyrrolo[1,2-a]pyrazin-7-yl}-1-benzothiophen-2-yl)ethyl]-6-methoxy-1,4-oxazocane-2-carboxamide and (2S,6S)—N-[(1R)-1-cyano-2-(5-{1,3-dimethylpyrrolo[1,2-a]pyrazin-7-yl}-1-benzothiophen-2-yl)ethyl]-6-methoxy-1,4-oxazocane-2-carboxamide

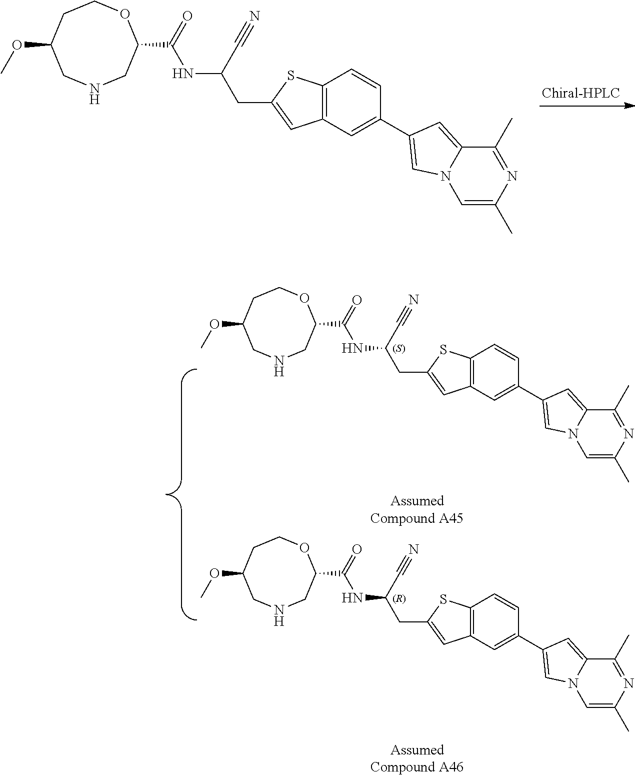

(2S,6S)—N-[1-cyano-2-(5-{1,3-dimethylpyrrolo[1,2-a]pyrazin-7-yl}-1-benzothiophen-2-yl)ethyl]-6-methoxy-1,4-oxazocane-2-carboxamide (35 mg, 0.07 mmol, 1.0 equiv) was separated by Prep-Chiral-HPLC with the following conditions (Column: XA-CHIRALPAK IE, 3*25 cm, 5 m; Mobile Phase A: HEX:DCM=3:1-HPLC, Mobile Phase B: EtOH (0.1% DEA); Flow rate: 35 mL/min; Gradient: isocratic 50; Wave Length: 254 nm; RT1(min): 24.8; RT2(min): 31.3; Sample Solvent: EtOH:DCM=1:1-HPLC; Injection Volume: 1.5 mL; Number Of Runs: 5) to afford Compound A45 (11 mg, 31%) as a white solid and Compound A46 (11 mg, 31%) as a white solid.

Compound A45: LCMS (ES) [M+H]⁺ m/z: 518.5. ¹H NMR (400 MHz, DMSO-d₆) δ 8.75 (d, J=8.5 Hz, 1H), 8.17 (d, J=1.7 Hz, 1H), 8.10 (d, J=1.6 Hz, 1H), 7.96-7.93 (m, 2H), 7.73 (dd, J=8.4, 1.8 Hz, 1H), 7.31 (s, 1H), 7.26 (t, J=1.3 Hz, 1H), 5.11-5.04 (m, 1H), 3.96 (dd, J=10.1, 2.6 Hz, 1H), 3.88-3.78 (m, 1H), 3.76-3.67 (m, 1H), 3.58-3.44 (m, 2H), 3.27-3.23 (m, 1H), 3.20 (s, 3H), 3.15 (dd, J=14.3, 2.2 Hz, 1H), 3.03 (dd, J=13.6, 2.7 Hz, 1H), 2.69 (dd, J=14.7, 3.8 Hz, 1H), 2.59 (s, 3H), 2.29 (s, 3H), 2.26-2.12 (m, 2H), 1.64 (dd, J=14.3, 3.5 Hz, 1H).

Compound A46: LCMS (ES) [M+H]⁺ m/z: 518.5. ¹H NMR (400 MHz, DMSO-d₆) δ 8.79 (d, J=8.5 Hz, 1H), 8.18 (d, J=1.8 Hz, 1H), 8.10 (d, J=1.6 Hz, 1H), 7.98-7.90 (m, 2H), 7.73 (dd, J=8.4, 1.7 Hz, 1H), 7.32 (s, 1H), 7.26 (t, J=1.3 Hz, 1H), 5.08-5.01 (m, 1H), 3.92-3.87 (m, 1H), 3.84 (dd, J=11.9, 2.9 Hz, 1H), 3.68 (dt, J=12.2, 4.0 Hz, 1H), 3.60-3.44 (m, 2H), 3.29-3.10 (m, 6H), 2.73 (dd, J=14.7, 3.6 Hz, 1H), 2.59 (s, 3H), 2.39 (dd, J=13.7, 10.2 Hz, 1H), 2.29 (s, 3H), 2.26-2.11 (m, 1H), 1.67-1.62 (m, 1H).

### Example 37: Synthesis of (2S,7R)—N-[(1S)-1-cyano-2-(4-{1,3-dimethylpyrrolo[1,2-a]pyrazin-7-yl}-2-fluorophenyl)ethyl]-7-methoxy-1,4-oxazocane-2-carboxamide (Compound A47)

### Step 1. Synthesis of tert-butyl (2S,7R)-2-{[(1S)-1-cyano-2-(4-{1,3-dimethylpyrrolo[1,2-a]pyrazin-7-yl}-2-fluorophenyl)ethyl]carbamoyl}-7-methoxy-1,4-oxazocane-4-carboxylate

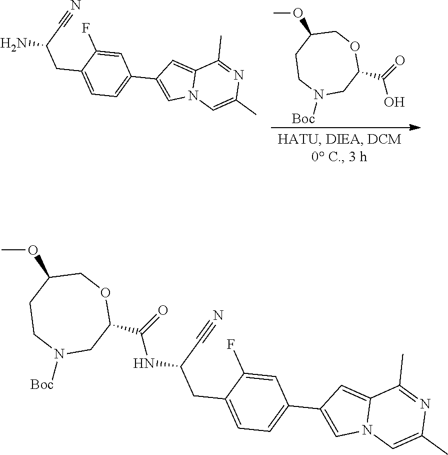

To a stirred mixture of (2S,7R)-4-(tert-butoxycarbonyl)-7-methoxy-1,4-oxazocane-2-carboxylic acid (101 mg, 0.35 mmol, 1.2 equiv), (2S)-2-amino-3-(4-{1,3-dimethylpyrrolo[1,2-a]pyrazin-7-yl}-2-fluorophenyl)propanenitrile (90 mg, 0.29 mmol, 1.0 equiv) and DIEA (113 mg, 0.87 mmol, 3.0 equiv) in DCM (5 mL) were added HATU (133 mg, 0.35 mmol, 1.2 equiv) in portions at 0° C. The resulting mixture was stirred for additional 3 h at 0° C. The resulting mixture was concentrated under reduced pressure. The residue was purified by silica gel column chromatography, eluted with PE/THF (1:1) to afford tert-butyl (2S,7R)-2-{[(1S)-1-cyano-2-(4-{1,3-dimethylpyrrolo[1,2-a]pyrazin-7-yl}-2-fluorophenyl)ethyl]carbamoyl}-7-methoxy-1,4-oxazocane-4-carboxylate (150 mg) as a light yellow oil. LCMS (ES, m/z): [M+H]⁺: 580.

### Step 2. Synthesis of (2S,7R)—N-[(1S)-1-cyano-2-(4-{1,3-dimethylpyrrolo[1,2-a]pyrazin-7-yl}-2-fluorophenyl)ethyl]-7-methoxy-1,4-oxazocane-2-carboxamide

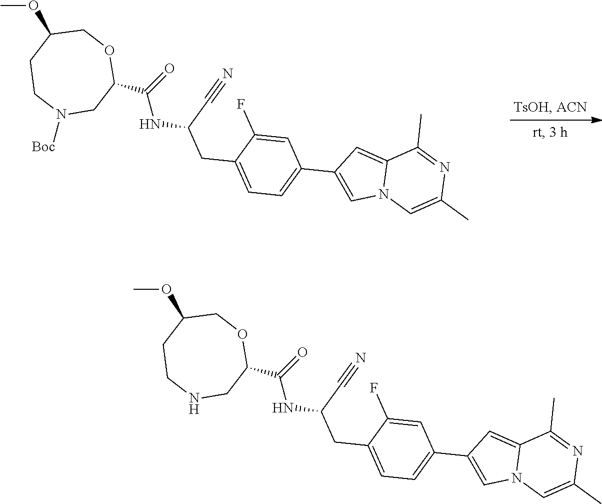

Into a 25 mL round-bottom flask were added tert-butyl (2S,7R)-2-{[(1S)-1-cyano-2-(4-{1,3-dimethylpyrrolo[1,2-a]pyrazin-7-yl}-2-fluorophenyl)ethyl]carbamoyl}-7-methoxy-1,4-oxazocane-4-carboxylate (140 mg, 0.24 mmol, 1.0 equiv), TsOH (124 mg, 0.72 mmol, 3.0 equiv) and ACN (5 mL) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 60% gradient in 12 min; detector, UV 254 nm. This resulted in Compound A47 (20 mg, 17%) as a white solid. LCMS (ES, m/z): [M+H]⁺: 480.2. ¹H NMR (400 MHz, DMSO-d₆): δ 8.72 (d, J=8.6 Hz, 1H), 8.10 (d, J=1.6 Hz, 1H), 7.90 (s, 1H), 7.65-7.54 (m, 2H), 7.38 (t, J=7.9 Hz, 1H), 7.26 (t, J=1.4 Hz, 1H), 5.03 (q, J=8.3 Hz, 1H), 4.11 (dd, J=11.4, 4.3 Hz, 1H), 3.92 (dd, J=9.0, 3.0 Hz, 1H), 3.59 (dd, J=11.5, 8.6 Hz, 1H), 3.42-3.33 (m, 1H), 3.30-3.23 (m, 4H), 3.16 (dd, J=13.6, 8.4 Hz, 1H), 3.01 (dd, J=14.5, 3.4 Hz, 2H), 2.57 (s, 3H), 2.49-2.44 (m, 1H), 2.40-2.30 (m, 1H), 2.28 (d, J=1.0 Hz, 3H), 1.97-1.87 (m, 1H), 1.65-1.53 (m, 1H).

### Example 38: Synthesis of (2S,6S)—N-[(1S)-1-cyano-2-(4-{1,3-dimethylpyrrolo[1,2-a]pyrazin-7-yl}-2-fluorophenyl)ethyl]-6-methoxy-1,4-oxazocane-2-carboxamide (Compound A48)

### Step 1. Synthesis of tert-butyl (2S,6S)-2-{[(1S)-1-cyano-2-(4-{1,3-dimethylpyrrolo[1,2-a]pyrazin-7-yl}-2-fluorophenyl)ethyl]carbamoyl}-6-methoxy-1,4-oxazocane-4-carboxylate

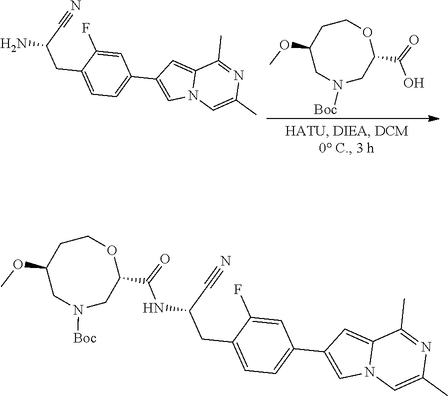

To a stirred solution of (2S)-2-amino-3-(4-{1,3-dimethylpyrrolo[1,2-a]pyrazin-7-yl}-2-fluorophenyl)propanenitrile (75 mg, 0.24 mmol, 1.0 equiv) and (2S,6S)-4-(tert-butoxycarbonyl)-6-methoxy-1,4-oxazocane-2-carboxylic acid (70 mg, 0.24 mmol, 1.0 equiv) in DCM (3 mL) was added HATU (110 mg, 0.29 mmol, 1.2 equiv) in portions at 0° C. The resulting mixture was stirred at 0° C. for 3 h. The reaction was concentrated to remove the solvent, the residue was purified by silica gel column chromatography, eluted with PE/THF (1:1) to afford tert-butyl (2S,6S)-2-{[(1S)-1-cyano-2-(4-{1,3-dimethylpyrrolo[1,2-a]pyrazin-7-yl}-2-fluorophenyl)ethyl]carbamoyl}-6-methoxy-1,4-oxazocane-4-carboxylate (140 mg, 99.3%) as a colorless semi-solid. LCMS (ES, m/z): [M+H]⁺: 580.

### Step 2. Synthesis of (2S,6S)—N-[(1S)-1-cyano-2-(4-{1,3-dimethylpyrrolo[1,2-a]pyrazin-7-yl}-2-fluorophenyl)ethyl]-6-methoxy-1,4-oxazocane-2-carboxamide

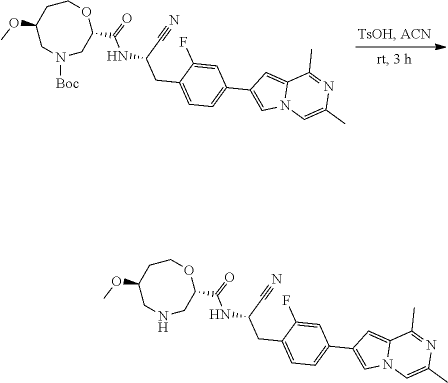

Into a 25 mL round-bottom flask were added tert-butyl (2S,6S)-2-{[(1S)-1-cyano-2-(4-{1,3-dimethylpyrrolo[1,2-a]pyrazin-7-yl}-2-fluorophenyl)ethyl]carbamoyl}-6-methoxy-1,4-oxazocane-4-carboxylate (140 mg, 0.24 mmol, 1.0 equiv), ACN (5 mL) and TsOH·H₂O (137 mg, 0.72 mmol, 3.0 equiv) at room temperature. The resulting mixture was stirred at room temperature for additional 3 h. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A48 (20.5 mg, 17.7%) as a white solid. LCMS (ES, m/z): [M+H]⁺: 480.2. ¹H NMR (400 MHz, DMSO-d₆) δ 8.70 (d, J=8.6 Hz, 1H), 8.10 (d, J=1.6 Hz, 1H), 7.90 (s, 1H), 7.64-7.54 (m, 2H), 7.36 (t, J=8.0 Hz, 1H), 7.26 (t, J=1.4 Hz, 1H), 5.00 (q, J=8.2 Hz, 1H), 3.92 (dd, J=10.1, 2.6 Hz, 1H), 3.87-3.76 (m, 1H), 3.73-3.64 (m, 1H), 3.29-3.21 (m, 2H), 3.20 (s, 3H), 3.17-3.11 (m, 2H), 3.01 (dd, J=13.6, 2.6 Hz, 1H), 2.68 (dd, J=14.7, 3.7 Hz, 1H), 2.57 (s, 3H), 2.28 (s, 3H), 2.23-2.14 (m, 2H), 1.66-1.60 (m, 1H).

### Example 39: Synthesis of (2S,6S)—N-[(1S)-1-cyano-2-[2-fluoro-4-(5,6,7,8-tetrahydro-1,5-naphthyridin-2-yl)phenyl]ethyl]-6-methoxy-1,4-oxazocane-2-carboxamide (Compound A49)

### Step 1. Synthesis of tert-butyl (2S,6S)-2-{[(1S)-1-cyano-2-[2-fluoro-4-(5,6,7,8-tetrahydro-1,5-naphthyridin-2-yl)phenyl]ethyl]carbamoyl}-6-methoxy-1,4-oxazocane-4-carboxylate

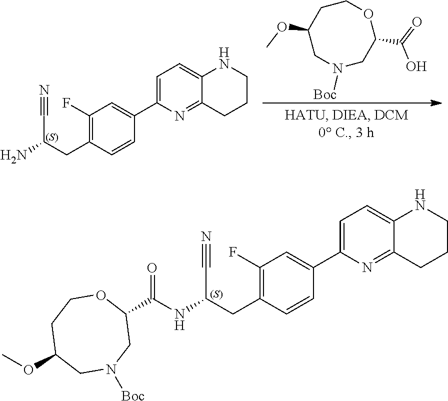

To a stirred solution of (2S,6S)-4-(tert-butoxycarbonyl)-6-methoxy-1,4-oxazocane-2-carboxylic acid (80 mg, 0.28 mmol, 1.0 equiv) and (2S)-2-amino-3-[2-fluoro-4-(5,6,7,8-tetrahydro-1,5-naphthyridin-2-yl)phenyl]propanenitrile (81 mg, 0.28 mmol, 1.0 equiv), DIEA (107 mg, 0.83 mmol, 3.0 equiv) in DCM (3 mL) was added HATU (126 mg, 0.33 mmol, 1.2 equiv) in portions at 0° C. The resulting mixture was stirred at 0° C. for 3 h. The residue was purified by silica gel column chromatography, eluted with PE/THF (1:1) to afford tert-butyl (2S,6S)-2-{[(1S)-1-cyano-2-[2-fluoro-4-(5,6,7,8-tetrahydro-1,5-naphthyridin-2-yl)phenyl]ethyl]carbamoyl}-6-methoxy-1,4-oxazocane-4-carboxylate (120 mg, 76.4%) as a white semi-solid. LCMS (ES, m/z): [M+H]⁺: 568.

### Step 2. Synthesis of (2S,6S)—N-[(1S)-1-cyano-2-[2-fluoro-4-(5,6,7,8-tetrahydro-1,5-naphthyridin-2-yl)phenyl]ethyl]-6-methoxy-1,4-oxazocane-2-carboxamide

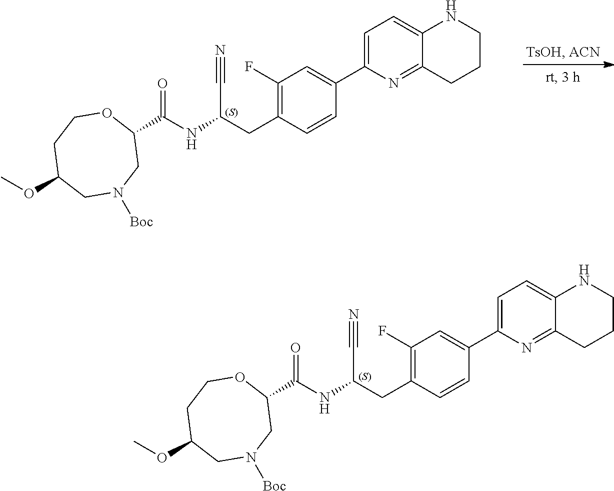

Into a 25 mL round-bottom flask were added tert-butyl (2S,6S)-2-{[(1S)-1-cyano-2-[2-fluoro-4-(5,6,7,8-tetrahydro-1,5-naphthyridin-2-yl)phenyl]ethyl]carbamoyl}-6-methoxy-1,4-oxazocane-4-carboxylate (120 mg, 0.21 mmol, 1.0 equiv) ACN (3 mL) and TsOH·H₂O (120 mg, 0.63 mmol, 3.0 equiv) at room temperature. The resulting mixture was stirred at room temperature for 3 h. The residue was purified by reversed-phase flash chromatography with the following conditions: column, C18 silica gel-120 g; mobile phase, MeCN in water (0.1% NH₃·H₂O), 10% to 50% gradient in 10 min; detector, UV 254 nm. This resulted in Compound A49 (18.9 mg, 19%) as a white solid. LCMS (ES, m/z): [M+H]⁺: 468.4. ¹H NMR (400 MHz, DMSO-d₆) δ 8.69 (d, J=8.6 Hz, 1H), 7.73-7.64 (m, 2H), 7.52 (d, J=8.4 Hz, 1H), 7.32 (t, J=8.2 Hz, 1H), 6.83 (d, J=8.4 Hz, 1H), 6.12 (t, J=2.3 Hz, 1H), 4.99 (q, J=8.2 Hz, 1H), 3.91 (dd, J=10.1, 2.6 Hz, 1H), 3.82 (td, J=11.9, 3.1 Hz, 1H), 3.69 (dt, J=12.1, 4.0 Hz, 1H), 3.29-3.08 (m, 9H), 3.00 (dd, J=13.6, 2.6 Hz, 1H), 2.84 (t, J=6.4 Hz, 2H), 2.68 (dd, J=14.7, 3.7 Hz, 1H), 2.25-2.14 (m, 2H), 1.95-1.90 (m, 2H), 1.66-1.59 (m, 1H).

### Example 40: Synthesis of (2S,7R)—N-[(1S)-1-cyano-2-[2-fluoro-4-(5,6,7,8-tetrahydro-1,5-naphthyridin-2-yl)phenyl]ethyl]-7-methoxy-1,4-oxazocane-2-carboxamide (Compound A50)

### Step 1. Synthesis of tert-butyl (2S,7R)-2-{[(1S)-1-cyano-2-[2-fluoro-4-(5,6,7,8-tetrahydro-1,5-naphthyridin-2-yl)phenyl]ethyl]carbamoyl}-7-methoxy-1,4-oxazocane-4-carboxylate

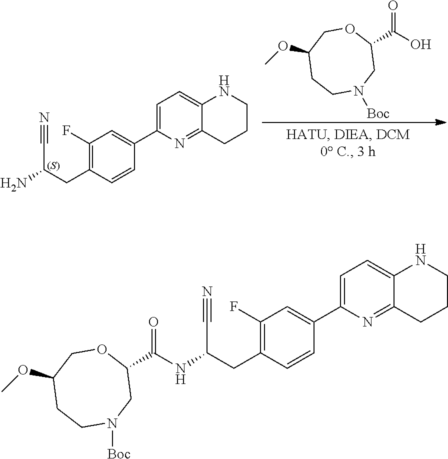

To a stirred solution of (2S,7R)-4-(tert-butoxycarbonyl)-7-methoxy-1,4-oxazocane-2-carboxylic acid (80 mg, 0.28 mmol, 1.0 equiv) and (2S)-2-amino-3-[2-fluoro-4-(5,6,7,8-tetrahydro-1,5-naphthyridin-2-yl)phenyl]propanenitrile (81 mg, 0.28 mmol, 1.0 equiv), DIEA (107 mg, 0.83 mmol, 3.0 equiv) in DCM (3 mL) was added HATU (126 mg, 0.33 mmol, 1.2 equiv) in portions at 0° C. The resulting mixture was stirred at 0° C. for 3 h. Concentrated to remove the solvent, the residue was purified by silica gel column chromatography, eluted with PE/THF (1:1) to afford tert-butyl (2S,7R)-2-{[(1S)-1-cyano-2-[2-fluoro-4-(5,6,7,8-tetrahydro-1,5-naphthyridin-2-yl)phenyl]ethyl]carbamoyl}-7-methoxy-1,4-oxazocane-4-carboxylate (130 mg, 82.8%) as a white semi-solid. LCMS (ES, m/z): [M+H]⁺: 568.

### Step 2. Synthesis of (2S,7R)—N-[(1S)-1-cyano-2-[2-fluoro-4-(5,6,7,8-tetrahydro-1,5-naphthyridin-2-yl)phenyl]ethyl]-7-methoxy-1,4-oxazocane-2-carboxamide

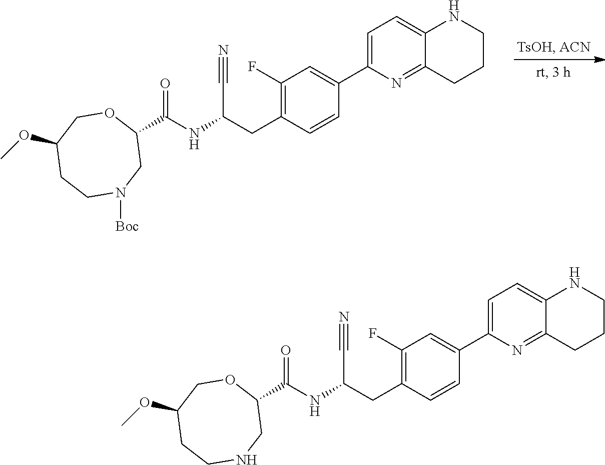 [SMILES: CO[C@@H]1CCN(C(=O)OC(C)(C)C)C[C@@H](C(=O)N[C@H](C#N)Cc2ccc(-c3ccc4c(n3)CCCN4)cc2F)OC1 ; CO[C@@H]1CCNC[C@@H](C(=O)N[C@H](C#N)Cc2ccc(-c3ccc4c(n3)CCCN4)cc2F)OC1]

Into a 25 mL round-bottom flask were added tert-butyl (2S,7R)-2-{[(1S)-1-cyano-2-[2-fluoro-4-(5,6,7,8-tetrahydro-1,5-naphthyridin-2-yl)phenyl]ethyl]carbamoyl}-7-methoxy-1,4-oxazocane-4-carboxylate (130 mg, 0.23 mmol, 1.0 equiv), ACN (3 mL) and TsOH·H₂O (130 mg, 0.69 mmol, 3.0 equiv) at room temperature. The resulting mixture was stirred at room temperature for 3 h. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound A50 (20.5 mg, 19%) as a white solid. LCMS (ES, m/z): [M+H]⁺: 468.2. ¹H NMR (400 MHz, DMSO-d₆) δ 8.70 (d, J=8.5 Hz, 1H), 7.75-7.64 (m, 2H), 7.56-7.49 (m, 1H), 7.33 (t, J=8.1 Hz, 1H), 6.83 (d, J=8.4 Hz, 1H), 6.12 (t, J=2.3 Hz, 1H), 5.01 (q, J=8.2 Hz, 1H), 4.11 (dd, J=11.6, 4.3 Hz, 1H), 3.92 (dd, J=9.1, 3.0 Hz, 1H), 3.63-3.49 (m, 1H), 3.40-3.34 (m, 1H), 3.28-3.08 (m, 7H), 3.07-2.96 (m, 2H), 2.84 (t, J=6.4 Hz, 2H), 2.49-32.46 (m, 1H), 2.35 (dd, J=14.4, 9.1 Hz, 1H), 1.97-1.86 (m, 3H), 1.63-1.54 (m, 1H).

### Example 41: Synthesis of (2S,7R)—N-[(1S)-1-cyano-2-(5-{1,3-dimethylpyrrolo[1,2-a]pyrazin-7-yl}-1-benzothiophen-2-yl)ethyl]-7-methoxy-1,4-oxazocane-2-carboxamide (Compound A51) and (2S,7R)—N-[(1R)-1-cyano-2-(5-{1,3-dimethylpyrrolo[1,2-a]pyrazin-7-yl}-1-benzothiophen-2-yl)ethyl]-7-methoxy-1,4-oxazocane-2-carboxamide (Compound A52)

### Step 1. Synthesis of 3-(5-{1,3-dimethylpyrrolo[1,2-a]pyrazin-7-yl}-1-benzothiophen-2-yl)-2-[(diphenylmethylidene)amino]propanenitrile

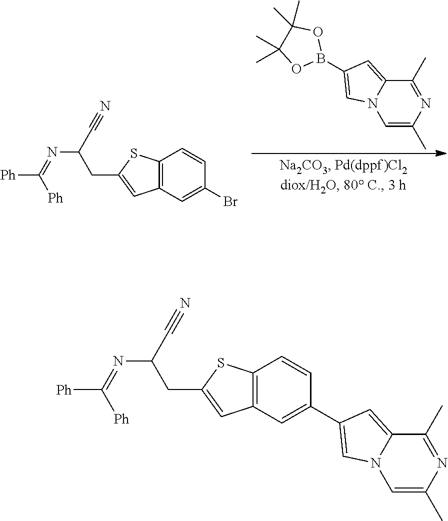

To a stirred solution of 3-(5-bromo-1-benzothiophen-2-yl)-2-[(diphenylmethylidene)amino]propanenitrile (550 mg, 1.24 mmol, 1.0 equiv) and 1,3-dimethyl-7-(4,4,5,5-tetramethyl-1,3,2-dioxaborolan-2-yl)pyrrolo[1,2-a]pyrazine (436 mg, 1.61 mmol, 1.3 equiv) in dioxane (6 mL) and H₂O (0.6 mL) were added Na₂CO₃ (261 mg, 2.47 mmol, 2.0 equiv) and Pd(dppf)Cl₂ (90 mg, 0.12 mmol, 0.1 equiv). The resulting mixture was stirred for 3 h at 80° C. under nitrogen atmosphere. The mixture was allowed to cool down to room temperature. The resulting mixture was concentrated under reduced pressure. The residue was purified by silica gel column chromatography, eluted with PE/EA (1:1) to afford 3-(5-{1,3-dimethylpyrrolo[1,2-a]pyrazin-7-yl}-1-benzothiophen-2-yl)-2-[(diphenylmethylidene)amino]propanenitrile (500 mg, 79%) as a light brown solid. LCMS (ES) [M+H]⁺ m/z: 511.

### Step 2. Synthesis of 2-amino-3-(5-{1,3-dimethylpyrrolo[1,2-a]pyrazin-7-yl}-1-benzothiophen-2-yl)propanenitrile

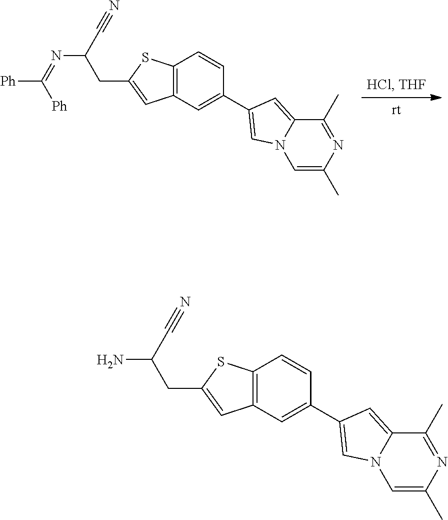 [SMILES: Cc1cn2cc(-c3ccc4sc(CC(C#N)N=C(c5ccccc5)c5ccccc5)cc4c3)cc2c(C)n1 ; Cc1cn2cc(-c3ccc4sc(CC(N)C#N)cc4c3)cc2c(C)n1]

To a stirred solution of 3-(5-{1,3-dimethylpyrrolo[1,2-a]pyrazin-7-yl}-1-benzothiophen-2-yl)-2-[(diphenylmethylidene)amino]propanenitrile (500 mg, 0.98 mmol, 1.0 equiv) in THF (10 mL) was added HCl (6M) (1 mL) dropwise at room temperature. The resulting mixture was stirred for 2 h at room temperature. The resulting mixture was extracted with Et₂O (2×10 mL). The aqueous layer was basified to pH 8 with saturated NaHCO₃ (aq.). The resulting mixture was extracted with EtOAc (3×20 mL). The combined organic layer was washed with brine (20 mL), dried over anhydrous Na₂SO₄. After filtration, the filtrate was concentrated under reduced pressure. This resulted in 2-amino-3-(5-{1,3-dimethylpyrrolo[1,2-a]pyrazin-7-yl}-1-benzothiophen-2-yl)propanenitrile (280 mg, 82%) as a light yellow solid. LCMS (ES) [M+H]⁺ m/z: 347.

### Step 3. Synthesis of tert-butyl (2S,7R)-2-{[1-cyano-2-(5-{1,3-dimethylpyrrolo[1,2-a]pyrazin-7-yl}-1-benzothiophen-2-yl)ethyl]carbamoyl}-7-methoxy-1,4-oxazocane-4-carboxylate

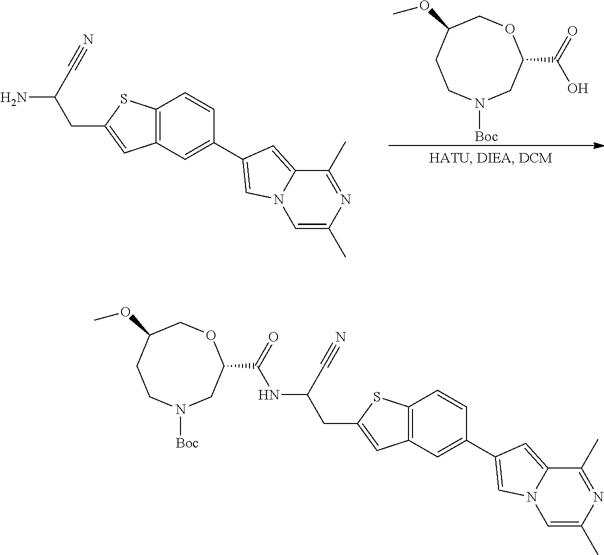

To a stirred solution of (2S,7R)-4-(tert-butoxycarbonyl)-7-methoxy-1,4-oxazocane-2-carboxylic acid (100 mg, 0.35 mmol, 1.0 equiv) and 2-amino-3-(5-{1,3-dimethylpyrrolo[1,2-a]pyrazin-7-yl}-1-benzothiophen-2-yl)propanenitrile (119 mg, 0.346 mmol, 1.0 equiv) in DCM (3 mL) were added DIEA (134 mg, 1.04 mmol, 3.0 equiv) and HATU (197 mg, 0.52 mmol, 1.5 equiv) in portions at 0° C. The resulting mixture was stirred for 1 h at 0° C. The resulting mixture was concentrated under reduced pressure. The residue was purified by silica gel column chromatography, eluted with PE/THF (2:1) to afford tert-butyl (2S,7R)-2-{[1-cyano-2-(5-{1,3-dimethylpyrrolo[1,2-a]pyrazin-7-yl}-1-benzothiophen-2-yl)ethyl]carbamoyl}-7-methoxy-1,4-oxazocane-4-carboxylate (180 mg, 84%) as a light yellow solid. (290 mg, 56%) as a colorless oil. LCMS (ES) [M+H]⁺ m/z: 618.

### Step 4. Synthesis of (2S,7R)—N-[1-cyano-2-(5-{1,3-dimethylpyrrolo[1,2-a]pyrazin-7-yl}-1-benzothiophen-2-yl)ethyl]-7-methoxy-1,4-oxazocane-2-carboxamide

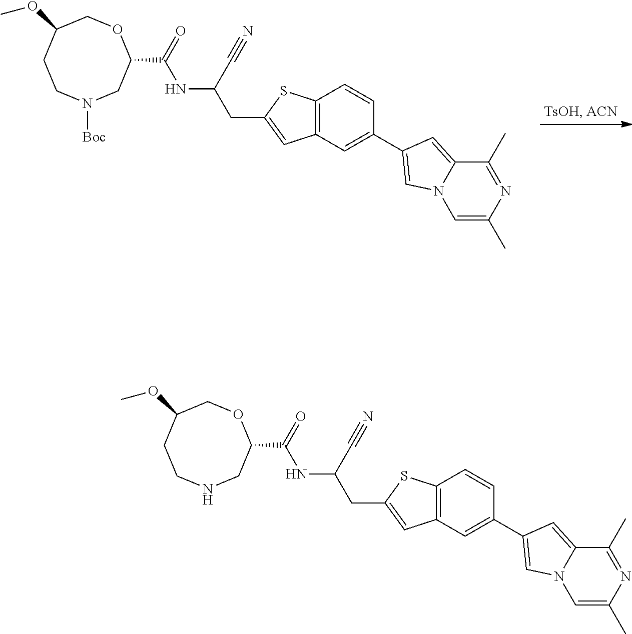

To a stirred solution of tert-butyl (2S,7R)-2-{[1-cyano-2-(5-{1,3-dimethylpyrrolo[1,2-a]pyrazin-7-yl}-1-benzothiophen-2-yl)ethyl]carbamoyl}-7-methoxy-1,4-oxazocane-4-carboxylate (180 mg, 0.29 mmol, 1.0 equiv) in ACN (3 mL) was added TsOH (150 mg, 0.87 mmol, 3.0 equiv). The resulting mixture was stirred for 3 h at room temperature. The mixture was purified by reversed-phase flash chromatography with the following conditions: column, C18 silica gel-120 g; mobile phase, MeCN in Water (0.1% NH₃·H₂O), 10% to 55% gradient in 15 min; detector, UV 254 nm. This resulted in (2S,7R)—N-[1-cyano-2-(5-{1,3-dimethylpyrrolo[1,2-a]pyrazin-7-yl}-1-benzothiophen-2-yl)ethyl]-7-methoxy-1,4-oxazocane-2-carboxamide (90 mg, 60%) as a white solid. LCMS (ES) [M+H]⁺ m/z: 518.

### Step 5. Synthesis of (2S,7R)—N-[(1S)-1-cyano-2-(5-{1,3-dimethylpyrrolo[1,2-a]pyrazin-7-yl}-1-benzothiophen-2-yl)ethyl]-7-methoxy-1,4-oxazocane-2-carboxamide and (2S,7R)—N-[(1R)-1-cyano-2-(5-{1,3-dimethylpyrrolo[1,2-a]pyrazin-7-yl}-1-benzothiophen-2-yl)ethyl]-7-methoxy-1,4-oxazocane-2-carboxamide

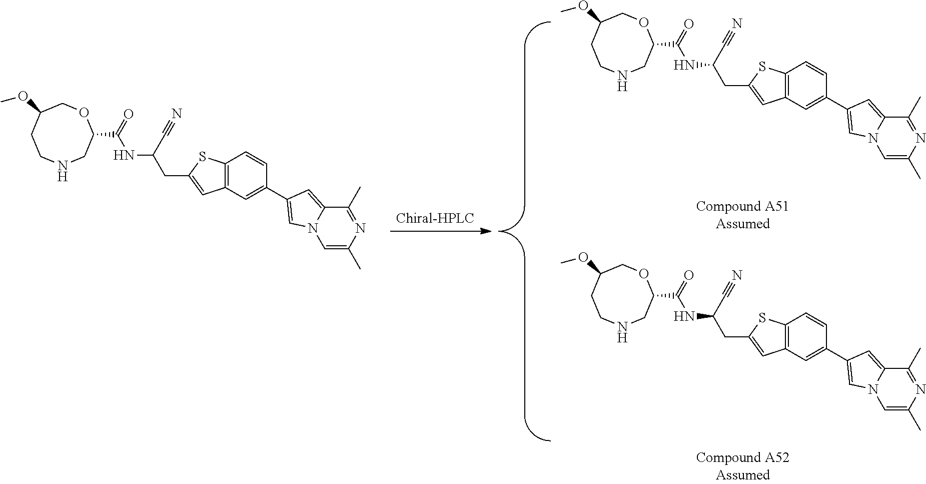

The (2S,7R)—N-[1-cyano-2-(5-{1,3-dimethylpyrrolo[1,2-a]pyrazin-7-yl}-1-benzothiophen-2-yl)ethyl]-7-methoxy-1,4-oxazocane-2-carboxamide (80 mg, 0.16 mmol, 1.0 equiv) was separated by Chiral-Prep-HPLC with the following conditions: Column: CHIRALPAK IE, 3*25 cm, 5 m; Mobile Phase A: Hex:DCM=2:1-HPLC, Mobile Phase B: EtOH:MeOH=1:2-HPLC; Flow rate: 35 mL/min; Gradient: isocratic 50; Wave Length: 254 nm; RT1(min): 15.4; RT2(min): 21.4; Sample Solvent: ETOH; Injection Volume: 1 mL; Number Of Runs: 8) to afford Compound A51 (30 mg, 37%) as a white solid and Compound A52 (30 mg, 37%) as a white solid. LCMS (ES) [M+H]⁺ m/z: 518.

Compound A51: ¹H NMR (300 MHz, DMSO-d₆) δ 8.77 (d, J=8.6 Hz, 1H), 8.17 (d, J=1.7 Hz, 1H), 8.10 (d, J=1.5 Hz, 1H), 7.98-7.90 (m, 2H), 7.73 (dd, J=8.4, 1.7 Hz, 1H), 7.32 (s, 1H), 7.29-7.23 (m, 1H), 5.10 (q, J=8.1 Hz, 1H), 4.10 (dd, J=11.6, 4.3 Hz, 1H), 3.94 (dd, J=9.1, 2.9 Hz, 1H), 3.66-3.42 (m, 3H), 3.40-3.35 (m, 1H), 3.25 (s, 3H), 3.07-2.91 (m, 2H), 2.59 (s, 3H), 2.50-2.30 (m, 2H), 2.29 (s, 3H), 1.97-1.84 (m, 1H), 1.66-1.50 (m, 1H).

Compound A52: ¹H NMR (400 MHz, DMSO-d₆) δ 8.78 (d, J=8.4 Hz, 1H), 8.18 (d, J=1.7 Hz, 1H), 8.10 (d, J=1.5 Hz, 1H), 7.98-7.90 (m, 2H), 7.73 (dd, J=8.5, 1.7 Hz, 1H), 7.33 (s, 1H), 7.25 (s, 1H), 5.06 (q, J=8.0 Hz, 1H), 4.15 (dd, J=11.4, 4.1 Hz, 1H), 3.90 (dd, J=9.2, 3.0 Hz, 1H), 3.61-3.44 (m, 3H), 3.39-3.31 (m, 1H), 3.25 (s, 3H), 3.14 (dd, J=14.5, 3.0 Hz, 1H), 3.10-3.00 (m, 1H), 2.59 (s, 3H), 2.59-2.53 (m, 1H), 2.29 (s, 3H), 2.00-1.84 (m, 1H), 1.64-1.52 (m, 1H).

### Example 42: Synthesis of (2S)—N-[(1S)-1-cyano-2-[9-(pyrrolidin-3-yl)-6H-benzo[c]chromen-3-yl]ethyl]-1,4-oxazepane-2-carboxamide (Compound B1)

### Step 1. Synthesis of methyl 3-[(2-bromo-4-chlorophenyl)methoxy]benzoate

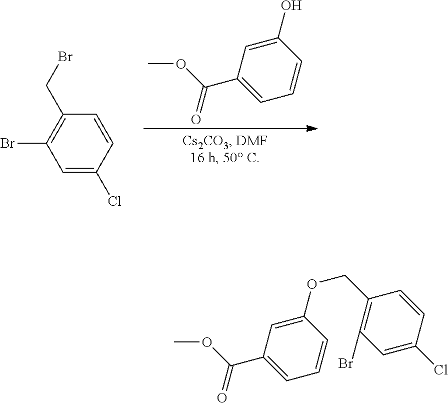 [SMILES: Clc1ccc(CBr)c(Br)c1 ; COC(=O)c1cccc(O)c1 ; COC(=O)c1cccc(OCc2ccc(Cl)cc2Br)c1]

To a stirred mixture of 2-bromo-1-(bromomethyl)-4-chlorobenzene (5.5 g, 19.34 mmol, 1.0 equiv) and methyl M-hydroxybenzoate (2.94 g, 19.34 mmol, 1.0 equiv) in DMF (70 mL) was added Cs₂CO₃ (12 g, 38.68 mmol, 2.0 equiv) in portions at room temperature. The resulting mixture was stirred for 16 h at 50° C. The mixture was allowed to cool down to room temperature. The resulting mixture was poured into the ice water (300 mL) slowly. The precipitated solid was collected by filtration to afford methyl 3-[(2-bromo-4-chlorophenyl)methoxy]benzoate (3.8 g, 55%) as a light yellow solid.

### Step 2. Synthesis of methyl 9-chloro-6H-benzo[c]chromene-3-carboxylate

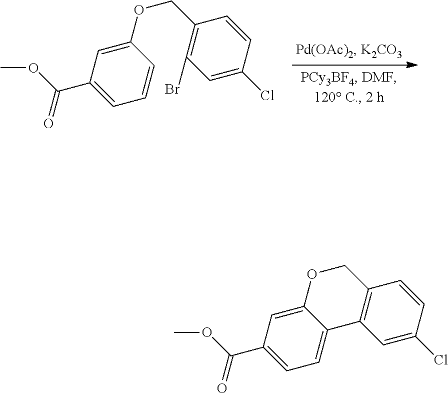 [SMILES: COC(=O)c1cccc(OCc2ccc(Cl)cc2Br)c1 ; COC(=O)c1ccc2c(c1)OCc1ccc(Cl)cc1-2]

Into a 100 mL round bottom flask were added methyl 3-[(2-bromo-4-chlorophenyl)methoxy]benzoate (5.5 g, 15.46 mmol, 1.0 equiv), DMF (60 mL), K₂CO₃ (4.28 g, 30.93 mmol, 2.0 equiv), PCy₃·HBF₄ (0.57 g, 1.54 mmol, 0.1 equiv) and Pd(OAc)₂ (0.35 g, 1.54 mmol, 0.10 equiv). The resulting mixture was stirred for 2 h at 120° C. under nitrogen atmosphere. The mixture was allowed to cool down to room temperature. The reaction was quenched with Water (80 mL) at 0° C. The resulting mixture was extracted with EA (3×100 mL). The combined organic layer was washed with brine (3×50 mL), dried over anhydrous Na₂SO₄. After filtration, the filtrate was concentrated under reduced pressure. The residue was purified by silica gel column chromatography, eluted with THF/PE (6%) to afford methyl 9-chloro-6H-benzo[c]chromene-3-carboxylate (3.3 g, 77.7%) as a light yellow solid.

### Step 3. Synthesis of methyl 9-chloro-6H-benzo[c]chromene-3-carboxylate

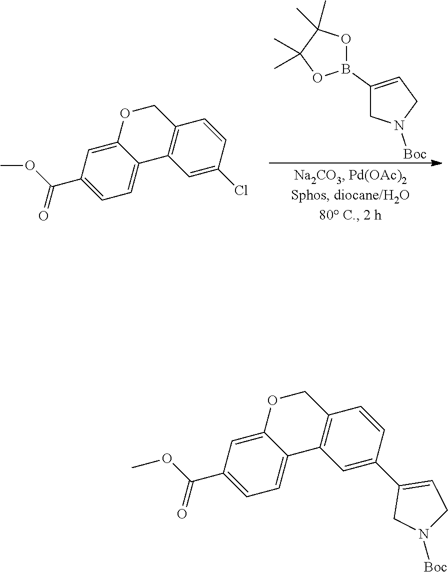 [SMILES: COC(=O)c1ccc2c(c1)OCc1ccc(Cl)cc1-2 ; CC(C)(C)OC(=O)N1CC=C(B2OC(C)(C)C(C)(C)O2)C1 ; COC(=O)c1ccc2c(c1)OCc1ccc(C3=CCN(C(=O)OC(C)(C)C)C3)cc1-2]

To a solution of methyl 9-chloro-6H-benzo[c]chromene-3-carboxylate (3.1 g, 11.28 mmol, 1.0 equiv) and tert-butyl 3-(4,4,5,5-tetramethyl-1,3,2-dioxaborolan-2-yl)-2,5-dihydropyrrole-1-carboxylate (3.33 g, 11.28 mmol, 1.0 equiv) in dioxane (50 mL) and H₂O (5 mL) were added Na₂CO₃ (2.39 g, 22.57 mmol, 2.0 equiv) and Pd(OAc)₂ (0.25 g, 1.12 mmol, 0.1 equiv) and SPhos (0.93 g, 2.25 mmol, 0.2 equiv). After stirring for 2 h at 80° C. under nitrogen atmosphere. The resulting mixture was concentrated under reduced pressure. The residue was purified by silica gel column chromatography, eluted with PE/EA (1:1) to afford tert-butyl 3-[3-(methoxycarbonyl)-6H-benzo[c]chromen-9-yl]-2,5-dihydropyrrole-1-carboxylate (3.5 g, 76%) as a light yellow solid. LCMS (ES, m/z): [M+H]⁺: 408.

### Step 4. Synthesis of tert-butyl 3-[3-(methoxycarbonyl)-6H-benzo[c]chromen-9-yl]pyrrolidine-1-carboxylate

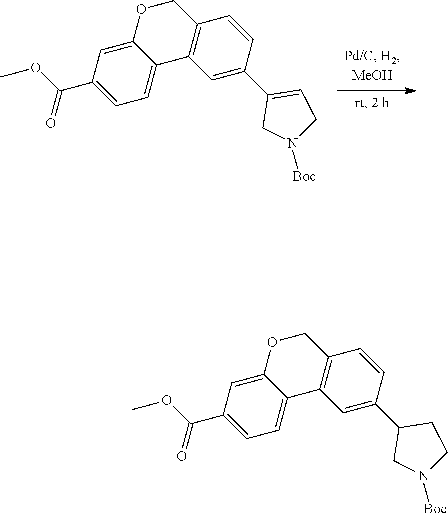 [SMILES: COC(=O)c1ccc2c(c1)OCc1ccc(C3=CCN(C(=O)OC(C)(C)C)C3)cc1-2 ; COC(=O)c1ccc2c(c1)OCc1ccc(C3CCN(C(=O)OC(C)(C)C)C3)cc1-2]

To a solution of tert-butyl 3-[3-(methoxycarbonyl)-6H-benzo[c]chromen-9-yl]-2,5-dihydropyrrole-1-carboxylate (3.4 g, 8.34 mmol, 1.0 equiv) in MeOH (50 mL) was added Pd/C (700 mg) in a pressure tank. The mixture was hydrogenated at room temperature under 10 atm of hydrogen pressure for 2 h. Filtered through a Celite pad and concentrated under reduced pressure. This resulted in tert-butyl 3-[3-(methoxycarbonyl)-6H-benzo[c]chromen-9-yl]pyrrolidine-1-carboxylate (3.2 g, 93%) as a light yellow solid and used to the next step without further purification. LCMS (ES, m/z): [M+H]⁺: 410.

### Step 5. Synthesis of tert-butyl 3-[3-(hydroxymethyl)-6H-benzo[c]chromen-9-yl]pyrrolidine-1-carboxylate

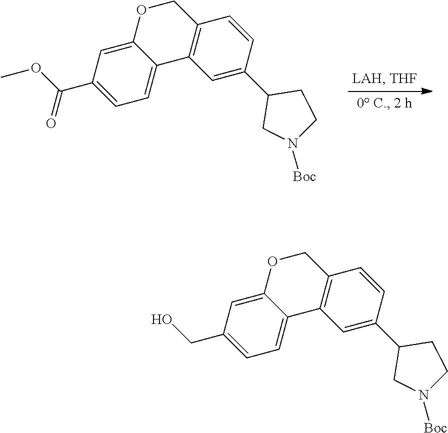 [SMILES: COC(=O)c1ccc2c(c1)OCc1ccc(C3CCN(C(=O)OC(C)(C)C)C3)cc1-2 ; CC(C)(C)OC(=O)N1CCC(c2ccc3c(c2)-c2ccc(CO)cc2OC3)C1]

To a stirred solution of tert-butyl 3-[3-(methoxycarbonyl)-6H-benzo[c]chromen-9-yl]pyrrolidine-1-carboxylate (2.2 g, 5.37 mmol, 1.0 equiv) and THF (30 mL) was added LAH (5.4 mL) (5.37 mL, 10.74 mmol, 2.0 equiv) (1M in THF) dropwise at 0° C. under nitrogen atmosphere. The resulting mixture was stirred for 2 h at 0° C. under nitrogen atmosphere. The reaction was quenched with Na₂SO₄·10H₂O at 0° C. The resulting mixture was filtered, the filter cake was washed with THF (3×20 mL). The filtrate was concentrated under reduced pressure. This resulted in tert-butyl 3-[3-(hydroxymethyl)-6H-benzo[c]chromen-9-yl]pyrrolidine-1-carboxylate (1.8 g, 87.8%) as a light yellow oil. LCMS (ES, m/z): [M+H]⁺: 382.

### Step 6. Synthesis of tert-butyl 3-[3-(bromomethyl)-6H-benzo[c]chromen-9-yl]pyrrolidine-1-carboxylate

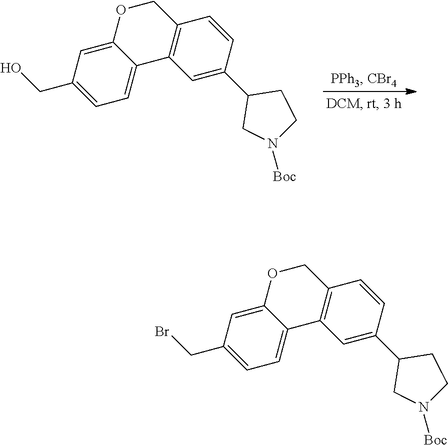 [SMILES: CC(C)(C)OC(=O)N1CCC(c2ccc3c(c2)-c2ccc(CO)cc2OC3)C1 ; CC(C)(C)OC(=O)N1CCC(c2ccc3c(c2)-c2ccc(CBr)cc2OC3)C1]

A solution of [(2-bromo-7-fluoro-1-benzothiophen-6-yl)methoxy](tert-butyl)dimethylsilane (2 g, 5.32 mmol, 1.0 equiv) in DCM (30 mL) was followed by the addition of CBr₄ (1.88 g, 5.66 mmol, 1.2 equiv) and PPh₃ (1.49 g, 5.66 mmol, 1.2 equiv) at room temperature. The resulting mixture was stirred for additional 3 h at room temperature. The reaction was quenched by the addition of Water (30 mL) at 0° C. The resulting mixture was extracted with DCM (3×50 mL). The combined organic layer was washed with brine (50 mL), dried over anhydrous Na₂SO₄. After filtration, the filtrate was concentrated under reduced pressure. The residue was purified by silica gel column chromatography, eluted with PE/EA (5:1) to afford tert-butyl 3-[3-(bromomethyl)-6H-benzo[c]chromen-9-yl]pyrrolidine-1-carboxylate (1.2 g, 57%) as a light yellow oil. LCMS (ES, m/z): [M+H]⁺: 444.

### Step 7. Synthesis of tert-butyl 3-(3-{2-cyano-2-[(diphenylmethylidene)amino]ethyl}-6H-benzo[c]chromen-9-yl)pyrrolidine-1-carboxylate

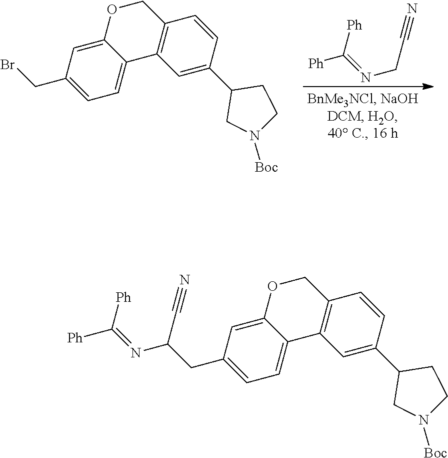 [SMILES: CC(C)(C)OC(=O)N1CCC(c2ccc3c(c2)-c2ccc(CBr)cc2OC3)C1 ; N#CCN=C(c1ccccc1)c1ccccc1 ; CC(C)(C)OC(=O)N1CCC(c2ccc3c(c2)-c2ccc(CC(C#N)N=C(c4ccccc4)c4ccccc4)cc2OC3)C1]

A solution of tert-butyl 3-[3-(bromomethyl)-6H-benzo[c]chromen-9-yl]pyrrolidine-1-carboxylate (1.2 g, 2.70 mmol, 1.0 equiv) in DCM (20 mL) was added 2-[(diphenylmethylidene)amino]acetonitrile (0.59 g, 2.70 mmol, 1.0 equiv), benzyltrimethylazanium chloride (0.05 g, 0.27 mmol, 0.1 equiv), NaOH (0.22 g, 5.40 mmol, 2.0 equiv) in H₂O (2 mL) was stirred for 16 h at 40° C. The resulting mixture was extracted with CH₂Cl₂ (3×100 mL). The combined organic layer was washed with brine (3×50 mL), dried over anhydrous Na₂SO₄. After filtration, the filtrate was concentrated under reduced pressure. The residue was purified by silica gel column chromatography, eluted with PE/EA (5:1) to afford tert-butyl 3-(3-{2-cyano-2-[(diphenylmethylidene)amino]ethyl}-6H-benzo[c]chromen-9-yl)pyrrolidine-1-carboxylate (1.5 g, 95%) as a light yellow solid. LCMS (ES, m/z): [M+H]⁺: 584.

### Step 8. Synthesis of tert-butyl 3-[3-(2-amino-2-cyanoethyl)-6H-benzo[c]chromen-9-yl]pyrrolidine-1-carboxylate

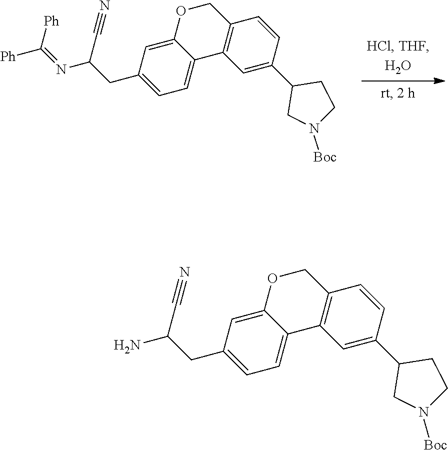 [SMILES: CC(C)(C)OC(=O)N1CCC(c2ccc3c(c2)-c2ccc(CC(C#N)N=C(c4ccccc4)c4ccccc4)cc2OC3)C1 ; CC(C)(C)OC(=O)N1CCC(c2ccc3c(c2)-c2ccc(CC(N)C#N)cc2OC3)C1]

Into a 100 mL round-bottom flask were added tert-butyl 3-(3-{2-cyano-2-[(diphenylmethylidene)amino]ethyl}-6H-benzo[c]chromen-9-yl)pyrrolidine-1-carboxylate (800 mg, 1.37 mmol, 1.0 equiv), THF (36 mL), H₂O (3.6 mL) and HCl (2 mL, 1 N in water) at room temperature. The resulting mixture was stirred for additional 2 h at room temperature. The mixture was basified to pH 12 with NaOH (2 N). The resulting mixture was extracted with EtOAc (3×50 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (1:2) to afford tert-butyl 3-[3-(2-amino-2-cyanoethyl)-6H-benzo[c]chromen-9-yl]pyrrolidine-1-carboxylate (500 mg, 87%) as a white solid. LCMS (ES, m/z): [M+H]⁺: 420.

### Step 9. Synthesis of tert-butyl (2S)-2-{[(1S)-2-{9-[1-(tert-butoxycarbonyl)pyrrolidin-3-yl]-6H-benzo[c]chromen-3-yl}-1-cyanoethyl]carbamoyl}-1,4-oxazepane-4-carboxylate

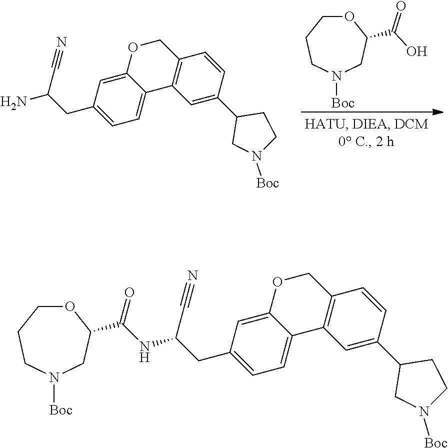 [SMILES: CC(C)(C)OC(=O)N1CCC(c2ccc3c(c2)-c2ccc(CC(N)C#N)cc2OC3)C1 ; CC(C)(C)OC(=O)N1CCCO[C@H](C(=O)O)C1 ; CC(C)(C)OC(=O)N1CCC(c2ccc3c(c2)-c2ccc(C[C@@H](C#N)NC(=O)[C@@H]4CN(C(=O)OC(C)(C)C)CCCO4)cc2OC3)C1]

To a stirred mixture of tert-butyl 3-[3-(2-amino-2-cyanoethyl)-6H-benzo[c]chromen-9-yl]pyrrolidine-1-carboxylate (123 mg, 0.29 mmol, 1.2 equiv), (2S)-4-(tert-butoxycarbonyl)-1,4-oxazepane-2-carboxylic acid (60 mg, 0.24 mmol, 1.0 equiv) and DIEA (94 mg, 0.75 mmol, 3.0 equiv) in DCM (5 mL) were added HATU (111 mg, 0.29 mmol, 1.2 equiv) in portions at 0° C. The resulting mixture was stirred for 2 h at 0° C. Concentrated to remove the solvent, the residue was purified by silica gel column chromatography, eluted with PE/THF (1:1) to afford tert-butyl (2S)-2-{[(1S)-2-{9-[1-(tert-butoxycarbonyl)pyrrolidin-3-yl]-6H-benzo[c]chromen-3-yl}-1-cyanoethyl]carbamoyl}-1,4-oxazepane-4-carboxylate (150 mg, 94%) as a white semi-solid. LCMS (ES, m/z): [M+H]⁺: 647.

### Step 10. Synthesis of (2S)—N-[(1S)-1-cyano-2-[9-(pyrrolidin-3-yl)-6H-benzo[c]chromen-3-yl]ethyl]-1,4-oxazepane-2-carboxamide

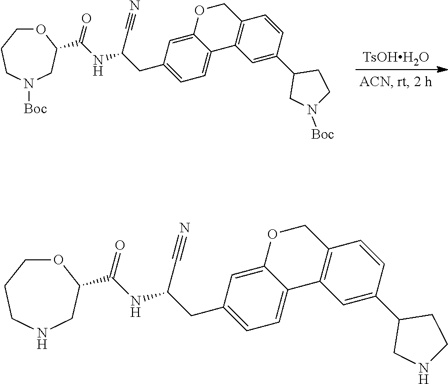 [SMILES: CC(C)(C)OC(=O)N1CCC(c2ccc3c(c2)-c2ccc(C[C@@H](C#N)NC(=O)[C@@H]4CN(C(=O)OC(C)(C)C)CCCO4)cc2OC3)C1 ; "TsOH·H2O" ; N#C[C@H](Cc1ccc2c(c1)OCc1ccc(C3CCNC3)cc1-2)NC(=O)[C@@H]1CNCCCO1]

Into a 25 mL round-bottom flask were added tert-butyl (2S)-2-[(2-{9-[1-(tert-butoxycarbonyl)pyrrolidin-3-yl]-6H-benzo[c]chromen-3-yl}-1-cyanoethyl)carbamoyl]-1,4-oxazepane-4-carboxylate (150 mg, 0.23 mmol, 1.0 equiv), TsOH·H₂O (176 mg, 0.92 mmol, 4.0 equiv) and ACN (4 mL) at room temperature. The resulting mixture was stirred for 2 h at room temperature. The reaction was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound B1 (27 mg, 26%) as white solid. LCMS (ES, m/z): [M+H]⁺: 447.5. ¹H NMR (400 MHz, DMSO-d₆) δ 8.56 (t, J=7.7 Hz, 1H), 7.84-7.66 (m, 2H), 7.21-7.13 (m, 2H), 6.97 (d, J=8.0 Hz, 1H), 6.89 (s, 1H), 5.08-4.88 (m, 3H), 4.02-3.63 (m, 4H), 3.19-3.06 (m, 3H), 3.04-2.57 (m, 6H), 2.22-1.96 (m, 2H), 1.77-1.64 (m, 3H).

### Example 43: Synthesis of (2S)—N-{1-cyano-2-[2,8-difluoro-9-(oxetan-3-yl)-6H-benzo[c]chromen-3-yl]ethyl}-1,4-oxazepane-2-carboxamide (Compound B2)

### Step 1. Synthesis of methyl 4-(benzyloxy)-2-bromo-5-fluorobenzoate

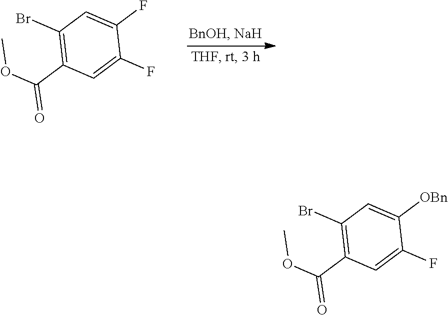 [SMILES: COC(=O)c1cc(F)c(F)cc1Br ; COC(=O)c1cc(F)c(OCc2ccccc2)cc1Br]

To a stirred solution of phenylmethanol (23.69 g, 219.100 mmol, 1.1 equiv) and in THF (500 mL) was added NaH (8.76 g, 219.100 mmol, 1.1 equiv, 60%). The resulting mixture was stirred for 2 h at 80° C. To the above mixture was added methyl 2-bromo-4,5-difluorobenzoate (50 g, 199.182 mmol, 1.0 equiv) in portions at 0° C. The resulting mixture was stirred for additional 3 h at room temperature. The reaction was quenched by the addition of Water/Ice (500 mL) at 0° C. The resulting mixture was extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (1:1) to afford methyl 4-(benzyloxy)-2-bromo-5-fluorobenzoate (36 g, 53.2%) as an off-white solid. ¹H NMR (300 MHz, DMSO-d6) δ 7.74 (d, J=11.7 Hz, 1H), 7.65 (d, J=7.8 Hz, 1H), 7.56-7.28 (m, 5H), 5.30 (s, 2H), 3.83 (s, 3H).

### Step 2. Synthesis of methyl 2-bromo-5-fluoro-4-hydroxybenzoate

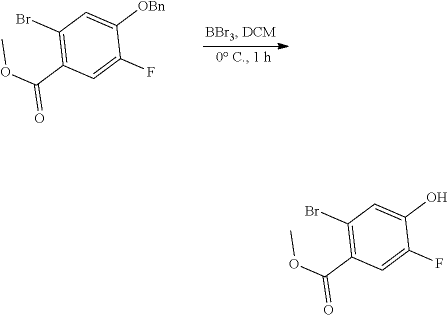 [SMILES: COC(=O)c1cc(F)c(OCc2ccccc2)cc1Br ; COC(=O)c1cc(F)c(O)cc1Br]

To a stirred solution of methyl 4-(benzyloxy)-2-bromo-5-fluorobenzoate (30 g, 88.454 mmol, 1.0 equiv) in DCM (500 mL) was added BBr₃ (97.30 mL, 97.299 mmol, 1.1 equiv) dropwise at 0° C. under nitrogen atmosphere. The resulting mixture was stirred for 1 h at 0° C. under nitrogen atmosphere. The reaction was quenched by the addition of Water/Ice (500 mL) at 0° C. The resulting mixture was extracted with CH₂Cl₂ (3×300 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (9:1) to afford methyl 2-bromo-5-fluoro-4-hydroxybenzoate (14 g, 63.5%) as a white solid. LCMS (ES, m/z): [M−H]⁻: 247. ¹H NMR (300 MHz, DMSO-d6) δ 11.24 (s, 1H), 7.69 (d, J=11.7 Hz, 1H), 7.27 (d, J=8.0 Hz, 1H), 3.80 (s, 3H).

### Step 3. Synthesis of methyl 2-bromo-5-fluoro-4-(methoxymethoxy)benzoate

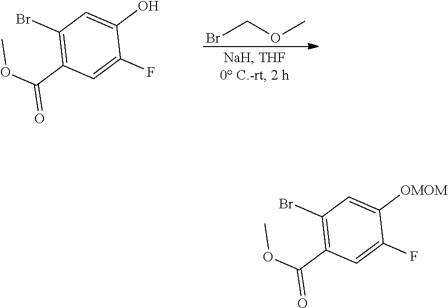

To a stirred solution of methyl 2-bromo-5-fluoro-4-hydroxybenzoate (13 g, 52.201 mmol, 1.0 equiv) in THF (200 mL) was added NaH (2.71 g, 67.861 mmol, 1.3 equiv, 60%) in portions at 0° C. under nitrogen atmosphere. The resulting mixture was stirred for 1 h at room temperature under nitrogen atmosphere. To the above mixture was added methane, bromomethoxy- (8.48 g, 67.861 mmol, 1.3 equiv) dropwise at 0° C. The resulting mixture was stirred for additional 1 h at room temperature. The reaction was quenched by the addition of Water/Ice (200 mL) at 0° C. The resulting mixture was extracted with EtOAc (3×200 mL). The combined organic layers were washed with brine (200 mL), dried over anhydrous Na₂SO₄. After filtration, the filtrate was concentrated under reduced pressure. The residue was purified by silica gel column chromatography, eluted with PE/EA (9:1) to afford methyl 2-bromo-5-fluoro-4-(methoxymethoxy)benzoate (14 g, 91.5%) as an off-white solid. ¹H NMR (300 MHz, Chloroform-d) δ 7.69 (d, J=11.4 Hz, 1H), 7.52 (d, J=7.5 Hz, 1H), 5.29 (s, 2H), 3.93 (s, 3H), 3.54 (s, 3H).

### Step 4. Synthesis of [2-bromo-5-fluoro-4-(methoxymethoxy)phenyl]methanol

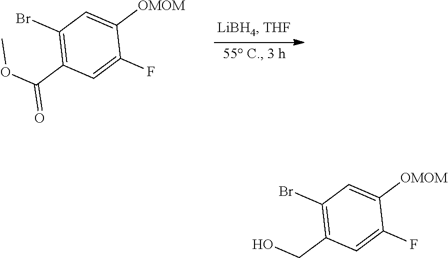

To a stirred solution of methyl 2-bromo-5-fluoro-4-(methoxymethoxy)benzoate (14 g, 47.767 mmol, 1.0 equiv) in THF (160 mL) and MeOH (40 mL) was added LiBH₄ (4.16 g, 191.068 mmol, 4.0 equiv) in portions at room temperature. The resulting mixture was stirred for 3 h at 55° C. The mixture was allowed to cool down to 0° C. The reaction was quenched by the addition of Water/Ice (200 mL) at 0° C. The resulting mixture was extracted with EtOAc (3×200 mL). The combined organic layers were washed with brine (200 mL), dried over anhydrous Na₂SO₄. After filtration, the filtrate was concentrated under reduced pressure. The residue was purified by silica gel column chromatography, eluted with PE/EA (1:1) to afford [2-bromo-5-fluoro-4-(methoxymethoxy)phenyl]methanol (9 g, 71.0%) as an off-white solid. ¹H NMR (300 MHz, DMSO-d6) δ 7.47 (d, J=7.7 Hz, 1H), 7.34 (d, J=12.2 Hz, 1H), 5.50 (t, J=5.6 Hz, 1H), 5.27 (s, 2H), 4.43 (d, J=5.6 Hz, 2H), 3.41 (s, 3H).

### Step 5. Synthesis of 1-bromo-2-(chloromethyl)-4-fluoro-5-(methoxymethoxy)benzene

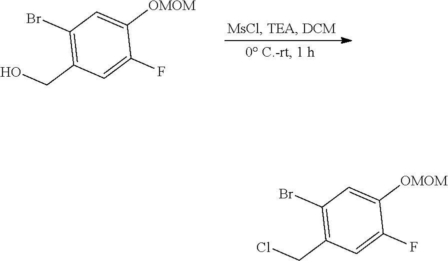

To a stirred solution of [2-bromo-5-fluoro-4-(methoxymethoxy)phenyl]methanol (9 g, 33.952 mmol, 1.0 equiv) and TEA (6.87 g, 67.904 mmol, 2.0 equiv) in DCM (150 mL) was added MsCl (4.67 g, 40.742 mmol, 1.2 equiv) dropwise at 0° C. under nitrogen atmosphere. The resulting mixture was stirred for 1 h at room temperature under nitrogen atmosphere. The resulting mixture was diluted with water (200 mL). The resulting mixture was extracted with CH₂Cl₂ (3×200 mL). The combined organic layers were washed with brine (200 mL), dried over anhydrous Na₂SO₄. After filtration, the filtrate was concentrated under reduced pressure. The crude product 1-bromo-2-(chloromethyl)-4-fluoro-5-(methoxymethoxy)benzene (12 g) was used in the next step directly without further purification.

### Step 6. Synthesis of methyl 5-{[2-bromo-5-fluoro-4-(methoxymethoxy)phenyl]methoxy}-2-fluorobenzoate

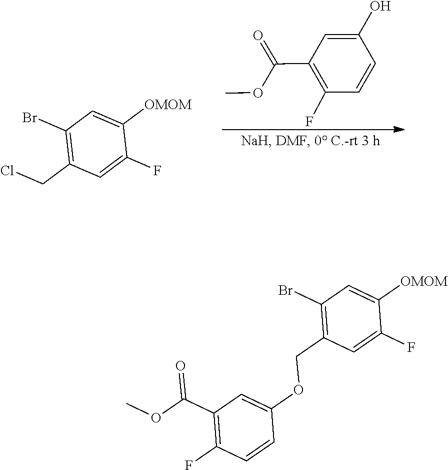

To a stirred solution of methyl 2-fluoro-5-hydroxybenzoate (12.96 g, 76.185 mmol, 1.8 equiv) in DMF (150 mL) was added NaH (2.20 g, 55.023 mmol, 1.3 equiv, 60%) in portions at 0° C. under nitrogen atmosphere. The resulting mixture was stirred for 1 h at room temperature under nitrogen atmosphere. To the above mixture was added 1-bromo-2-(chloromethyl)-4-fluoro-5-(methoxymethoxy)benzene (12 g, 42.325 mmol, 1.0 equiv) in portions at 0° C. The resulting mixture was stirred for additional 2 h at room temperature. The reaction was quenched by the addition of Water/Ice (500 mL) at 0° C. The resulting mixture was extracted with EtOAc (3×200 mL). The combined organic layers were washed with brine (2×200 mL), dried over anhydrous Na₂SO₄. After filtration, the filtrate was concentrated under reduced pressure. The residue was purified by trituration with PE:EA=: 1 (50 mL). This resulted in methyl 5-{[2-bromo-5-fluoro-4-(methoxymethoxy)phenyl]methoxy}-2-fluorobenzoate (9 g, 50.9%) as an off-white solid. LCMS (ES, m/z): [M+H]⁺: 417.

### Step 7. Synthesis of methyl 2,8-difluoro-9-(methoxymethoxy)-6H-benzo[c]chromene-3-carboxylate

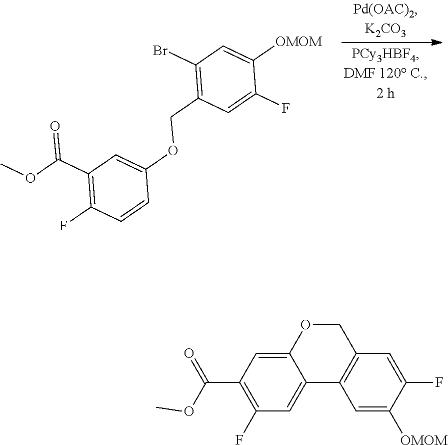

To a stirred solution of methyl 5-{[2-bromo-5-fluoro-4-(methoxymethoxy)phenyl]methoxy}-2-fluorobenzoate (9 g, 21.572 mmol, 1.0 equiv) and K₂CO₃ (5.96 g, 43.144 mmol, 2.0 equiv) in DMF (130 mL) were added PCy₃HBF₄ (0.60 g, 2.157 mmol, 0.1 equiv) and Pd(OAc)₂ (0.48 g, 2.157 mmol, 0.1 equiv). The resulting mixture was stirred for 2 h at 120° C. under nitrogen atmosphere. The mixture was allowed to cool down to room temperature. The resulting mixture was concentrated under reduced pressure. The residue was purified by silica gel column chromatography, eluted with PE/THF (19:1) to afford methyl 2,8-difluoro-9-(methoxymethoxy)-6H-benzo[c]chromene-3-carboxylate (6.4 g, 88.2%) as a light-yellow solid. LCMS (ES, m/z): [M+H]⁺: 337.

### Step 8. Synthesis of methyl 2,8-difluoro-9-hydroxy-6H-benzo[c]chromene-3-carboxylate

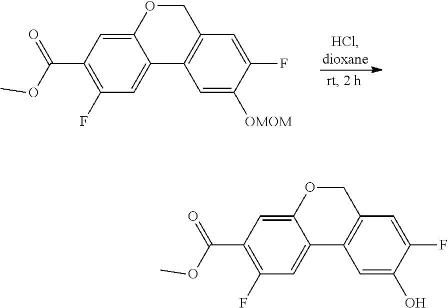

To a stirred solution of methyl 2,8-difluoro-9-(methoxymethoxy)-6H-benzo[c]chromene-3-carboxylate (6.4 g, 19.031 mmol, 1 equiv) in dioxane (60 mL) was added HCl (gas) in 1,4-dioxane (60 mL). The resulting mixture was stirred for 2 h at room temperature. The resulting mixture was concentrated under reduced pressure. The residue was purified by trituration with PE:EA=10:1 (30 mL). This resulted in methyl 2,8-difluoro-9-hydroxy-6H-benzo[c]chromene-3-carboxylate (5.2 g, 93.5%) as a white solid. LCMS (ES, m/z): [M+H]⁺: 293.

### Step 9. Synthesis of methyl 2,8-difluoro-9-(trifluoromethanesulfonyloxy)-6H-benzo[c]chromene-3-carboxylate

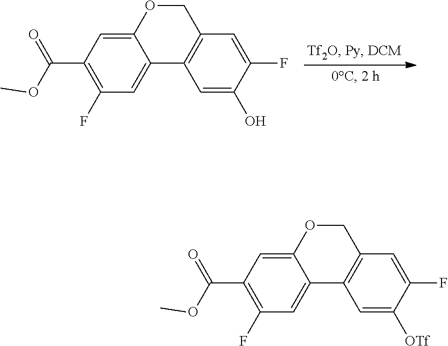 [SMILES: COC(=O)c1cc2c(cc1F)-c1cc(O)c(F)cc1CO2 ; COC(=O)c1cc2c(cc1F)-c1cc(OS(=O)(=O)C(F)(F)F)c(F)cc1CO2]

To a stirred solution of methyl 2,8-difluoro-9-hydroxy-6H-benzo[c]chromene-3-carboxylate (5.2 g, 17.794 mmol, 1 equiv) and Pyridine (5.63 g, 71.176 mmol, 4.0 equiv) in DCM (60 mL) were added Tf₂O (7.53 g, 26.691 mmol, 1.5 equiv) at 0° C. under nitrogen atmosphere. The resulting mixture was stirred for 2 h at 0° C. under nitrogen atmosphere. The resulting mixture was diluted with water (100 mL). The resulting mixture was extracted with CH₂Cl₂ (3×100 mL). The combined organic layers were washed with brine (100 mL), dried over anhydrous Na₂SO₄. After filtration, the filtrate was concentrated under reduced pressure. The residue was purified by trituration with PE:EA=20:1 (30 mL). This resulted in methyl 2,8-difluoro-9-(trifluoromethanesulfonyloxy)-6H-benzo[c]chromene-3-carboxylate (6.8 g, 90.0%) as a light-yellow solid. LCMS (ES, m/z): [M+H]⁺: 425.

### Step 10. Synthesis of methyl 2,8-difluoro-9-(4,4,5,5-tetramethyl-1,3,2-dioxaborolan-2-yl)-6H-benzo[c]chromene-3-carboxylate

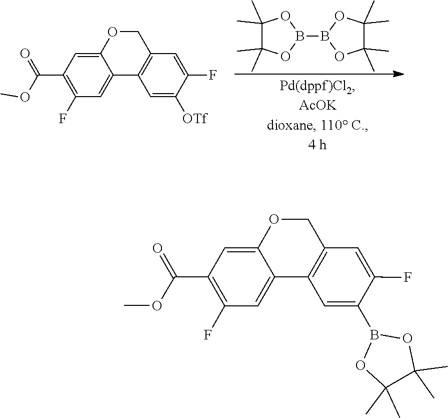

To a stirred solution of methyl 2,8-difluoro-9-(trifluoromethanesulfonyloxy)-6H-benzo[c]chromene-3-carboxylate (6.6 g, 15.555 mmol, 1.0 equiv) and bis(pinacolato)diboron (4.74 g, 18.666 mmol, 1.2 equiv) in dioxane (100 mL) were added Pd(dppf)Cl₂ (1.14 g, 1.556 mmol, 0.1 equiv) and KOAc (3.05 g, 31.110 mmol, 2.0 equiv). The resulting mixture was stirred for 4 h at 110° C. under nitrogen atmosphere. The mixture was allowed to cool down to room temperature. The resulting mixture was concentrated under reduced pressure. The residue was purified by silica gel column chromatography, eluted with PE/THF (15:1) to afford methyl 2,8-difluoro-9-(4,4,5,5-tetramethyl-1,3,2-dioxaborolan-2-yl)-6H-benzo[c]chromene-3-carboxylate (5.8 g, 92.7%) as a white solid. LCMS (ES, m/z): [M+H]⁺: 403.

### Step 11. Synthesis of methyl 9-bromo-2,8-difluoro-6H-benzo[c]chromene-3-carboxylate

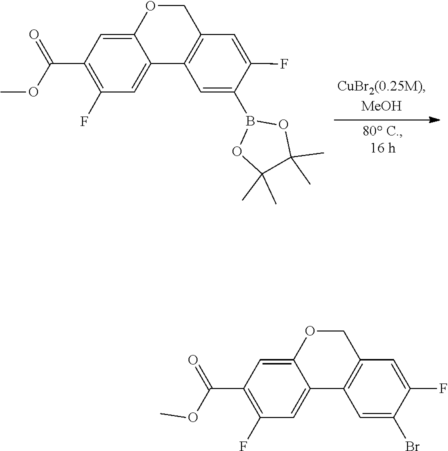

To a stirred solution of methyl 2,8-difluoro-9-(4,4,5,5-tetramethyl-1,3,2-dioxaborolan-2-yl)-6H-benzo[c]chromene-3-carboxylate (5.8 g, 14.421 mmol, 1.0 equiv) in MeOH (170 mL) were added CuBr₂ (9.66 g, 43.263 mmol, 3.0 equiv) in H₂O (165 mL). The resulting mixture was stirred for 16 h at 80° C. under nitrogen atmosphere. The mixture was allowed to cool down to room temperature. The resulting mixture was concentrated under reduced pressure. The resulting mixture was diluted with water (100 mL). The resulting mixture was extracted with EtOAc (3×60 mL). The combined organic layers were washed with brine (100 mL), dried over anhydrous Na₂SO₄. After filtration, the filtrate was concentrated under reduced pressure. This resulted in methyl 9-bromo-2,8-difluoro-6H-benzo[c]chromene-3-carboxylate (3.7 g, 72.2%) as a light brown solid. LCMS (ES, m/z): [M+H]⁺: 355.

### Step 12. Synthesis of methyl 2,8-difluoro-9-(oxetan-3-yl)-6H-benzo[c]chromene-3-carboxylate

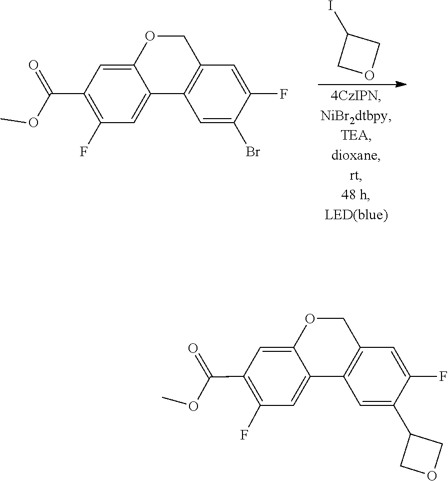 [SMILES: COC(=O)c1cc2c(cc1F)-c1cc(Br)c(F)cc1CO2 ; IC1COC1 ; COC(=O)c1cc2c(cc1F)-c1cc(C3COC3)c(F)cc1CO2]

To a stirred solution of methyl 9-bromo-2,8-difluoro-6H-benzo[c]chromene-3-carboxylate (3.2 g, 9.011 mmol, 1.0 equiv) and 3-iodooxetane (3.32 g, 18.022 mmol, 2.0 equiv) in dioxane (60 mL) were added TEA (2.74 g, 27.033 mmol, 3.0 equiv) and NiBr₂dtbpy (0.44 g, 0.901 mmol, 0.1 equiv) and 4CzIPN (0.36 g, 0.451 mmol, 0.05 equiv). The resulting mixture was stirred for 48 h at room temperature under nitrogen atmosphere and blue LED light. The resulting mixture was diluted with water (60 mL). The resulting mixture was extracted with EtOAc (3×40 mL). The combined organic layers were washed with brine (30 mL), dried over anhydrous Na₂SO₄. After filtration, the filtrate was concentrated under reduced pressure. The residue was purified by silica gel column chromatography, eluted with PE/THF (9:1) to afford methyl 2,8-difluoro-9-(oxetan-3-yl)-6H-benzo[c]chromene-3-carboxylate (1.6 g, 53.4%) as a white solid. LCMS (ES, m/z): [M+H]⁺: 333.

### Step 13. Synthesis of [2,8-difluoro-9-(oxetan-3-yl)-6H-benzo[c]chromen-3-yl]methanol

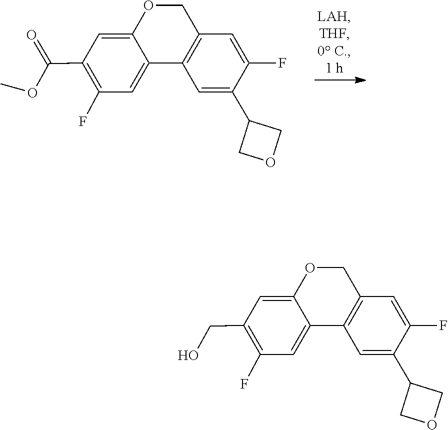 [SMILES: COC(=O)c1cc2c(cc1F)-c1cc(C3COC3)c(F)cc1CO2 ; OCc1cc2c(cc1F)-c1cc(C3COC3)c(F)cc1CO2]

To a stirred solution of methyl 2,8-difluoro-9-(oxetan-3-yl)-6H-benzo[c]chromene-3-carboxylate (1.6 g, 4.815 mmol, 1.0 equiv) in THF (30 mL) was added LAH (2.89 mL, 5.778 mmol, 1.2 equiv) dropwise 0° C. under nitrogen atmosphere. The resulting mixture was stirred for 1 h at 0° C. under nitrogen atmosphere. The resulting mixture was diluted with THF (30 mL). The reaction was quenched with Na₂SO₄·10H₂O at 0° C. The resulting mixture was filtered, the filter cake was washed with THF (3×10 mL). The filtrate was concentrated under reduced pressure. This resulted in [2,8-difluoro-9-(oxetan-3-yl)-6H-benzo[c]chromen-3-yl]methanol (1.1 g, 75.0%) as a light yellow solid. LCMS (ES, m/z): [M+H]⁺: 305.

### Step 14. Synthesis of [2,8-difluoro-9-(oxetan-3-yl)-6H-benzo[c]chromen-3-yl]methyl methanesulfonate

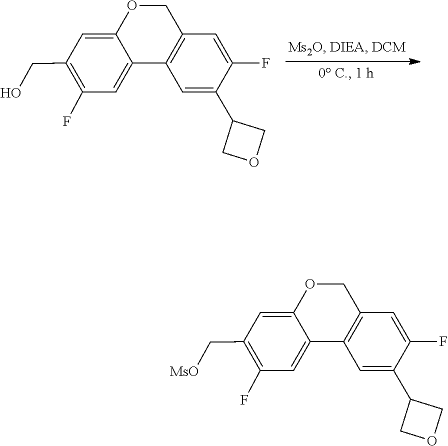 [SMILES: OCc1cc2c(cc1F)-c1cc(C3COC3)c(F)cc1CO2 ; CS(=O)(=O)OCc1cc2c(cc1F)-c1cc(C3COC3)c(F)cc1CO2]

To a stirred solution of [2,8-difluoro-9-(oxetan-3-yl)-6H-benzo[c]chromen-3-yl]methanol (1.1 g, 3.615 mmol, 1.0 equiv) and DIEA (1.87 g, 14.460 mmol, 4.0 equiv) in DCM (20 mL) were added methanesulfonyl methanesulfonate (1.26 g, 7.230 mmol, 2.0 equiv) in portions at 0° C. under nitrogen atmosphere. The resulting mixture was stirred for 1 h at 0° C. under nitrogen atmosphere. The resulting mixture was diluted with water (30 mL). The resulting mixture was extracted with CH₂Cl₂ (3×20 mL). The combined organic layers were washed with brine (20 mL), dried over anhydrous Na₂SO₄. After filtration, the filtrate was concentrated under reduced pressure. This resulted in [2,8-difluoro-9-(oxetan-3-yl)-6H-benzo[c]chromen-3-yl]methyl methanesulfonate (1.3 g, 94.0%) as a light yellow solid.

### Step 15. Synthesis of 3-[3-(bromomethyl)-2,8-difluoro-6H-benzo[c]chromen-9-yl]oxetane

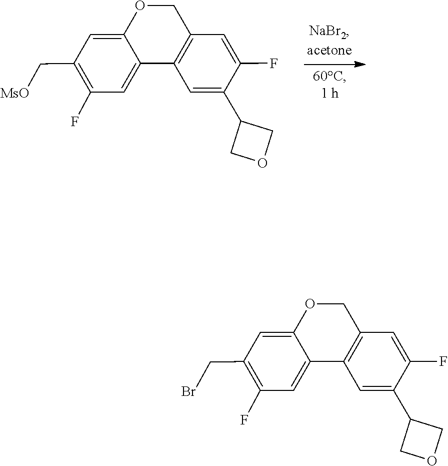 [SMILES: CS(=O)(=O)OCc1cc2c(cc1F)-c1cc(C3COC3)c(F)cc1CO2 ; Fc1cc2c(cc1CBr)OCc1cc(F)c(C3COC3)cc1-2]

To a stirred solution of [2,8-difluoro-9-(oxetan-3-yl)-6H-benzo[c]chromen-3-yl]methyl methanesulfonate (1.3 g, 3.400 mmol, 1.0 equiv) in acetone (30 mL) was added NaBr (3.50 g, 34.000 mmol, 10.0 equiv). The resulting mixture was stirred for 1 h at 60° C. The mixture was allowed to cool down to room temperature. The resulting mixture was diluted with water (30 mL). The resulting mixture was extracted with EtOAc (3×20 mL). The combined organic layers were washed with brine (20 mL), dried over anhydrous Na₂SO₄. After filtration, the filtrate was concentrated under reduced pressure. This resulted in 3-[3-(bromomethyl)-2,8-difluoro-6H-benzo[c]chromen-9-yl]oxetane (1.1 g, 88.1%) as a light yellow solid. ¹H NMR (400 MHz, Chloroform-d) δ 7.71 (d, J=6.9 Hz, 1H), 7.43 (d, J=10.1 Hz, 1H), 7.04 (d, J=6.5 Hz, 1H), 6.90 (d, J=9.5 Hz, 1H), 5.13 (dd, J=8.5, 6.0 Hz, 2H), 5.08 (s, 2H), 4.89 (t, J=6.5 Hz, 2H), 4.58 (t, J=7.8 Hz, 1H), 4.52 (s, 2H).

### Step 16. Synthesis of 3-[2,8-difluoro-9-(oxetan-3-yl)-6H-benzo[c]chromen-3-yl]-2-[(diphenylmethylidene)amino]propanenitrile

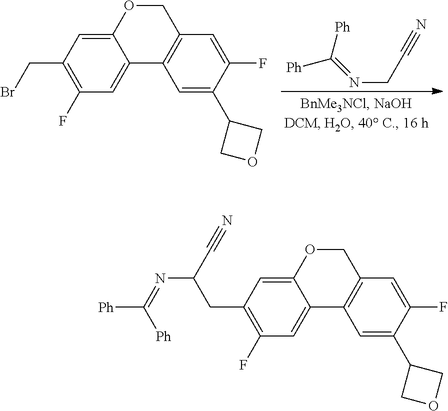

To a stirred solution of 3-[3-(bromomethyl)-2,8-difluoro-6H-benzo[c]chromen-9-yl]oxetane (1.1 g, 2.996 mmol, 1.0 equiv) and 2-[(diphenylmethylidene)amino]acetonitrile (0.66 g, 2.996 mmol, 1.0 equiv) in DCM (20 mL) and H₂O (2 mL) were added NaOH (0.24 g, 5.992 mmol, 2.0 equiv) and benzyltrimethylazanium chloride (0.06 g, 0.300 mmol, 0.1 equiv). The resulting mixture was stirred for 16 h at 40° C. The mixture was allowed to cool down to room temperature. The resulting mixture was diluted with water (30 mL). The resulting mixture was extracted with CH₂Cl₂ (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/THF (7:1) to afford 3-[2,8-difluoro-9-(oxetan-3-yl)-6H-benzo[c]chromen-3-yl]-2-[(diphenylmethylidene)amino]propanenitrile (1.1 g, 72.4%) as a light yellow solid. LCMS (ES, m/z): [M+H]⁺: 507.

### Step 17. Synthesis of 2-amino-3-[2,8-difluoro-9-(oxetan-3-yl)-6H-benzo[c]chromen-3-yl]propanenitrile

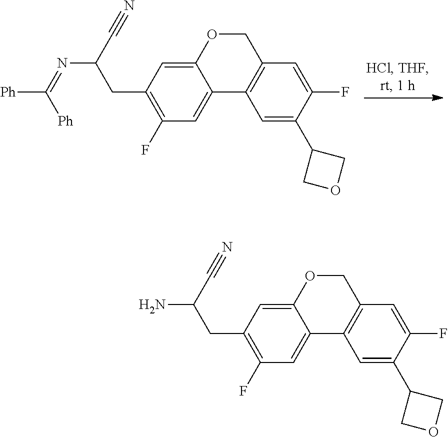 [SMILES: N#CC(Cc1cc2c(cc1F)-c1cc(C3COC3)c(F)cc1CO2)N=C(c1ccccc1)c1ccccc1 ; N#CC(N)Cc1cc2c(cc1F)-c1cc(C3COC3)c(F)cc1CO2]

To a stirred solution of 3-[2,8-difluoro-9-(oxetan-3-yl)-6H-benzo[c]chromen-3-yl]-2-[(diphenylmethylidene)amino]propanenitrile (1.1 g, 2.172 mmol, 1.0 equiv) in THF (50 mL) was added HCl (2.5 mL). The resulting mixture was stirred for 1 h at room temperature. The resulting mixture was diluted with water (50 mL). The resulting mixture was extracted with EtOEt (2×20 mL). The aqueous layer was basified to pH 10 with NaOH (aq.) (1N). The resulting mixture was extracted with CH₂Cl₂ (3×30 mL). The combined organic layers were washed with brine (20 mL), dried over anhydrous Na₂SO₄. After filtration, the filtrate was concentrated under reduced pressure. This resulted in 2-amino-3-[2,8-difluoro-9-(oxetan-3-yl)-6H-benzo[c]chromen-3-yl]propanenitrile (550 mg, 73.9%) as a white solid. LCMS (ES, m/z): [M+H]⁺: 343.

### Step 18. Synthesis of tert-butyl (2S)-2-({1-cyano-2-[2,8-difluoro-9-(oxetan-3-yl)-6H-benzo[c]chromen-3-yl]ethyl}carbamoyl)-1,4-oxazepane-4-carboxylate

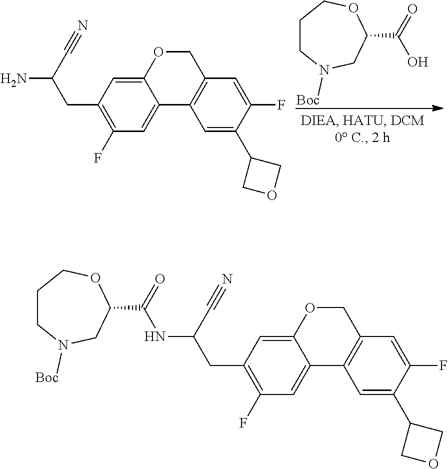

To a stirred solution of 2-amino-3-[2,8-difluoro-9-(oxetan-3-yl)-6H-benzo[c]chromen-3-yl]propanenitrile (550 mg, 1.607 mmol, 1.0 equiv) and (2S)-4-(tert-butoxycarbonyl)-1,4-oxazepane-2-carboxylic acid (394 mg, 1.607 mmol, 1.00 equiv) in DCM (10 mL) were added DIEA (622 mg, 4.821 mmol, 3.0 equiv) and HATU (733 mg, 1.928 mmol, 1.2 equiv) in portions at 0° C. under nitrogen atmosphere. The resulting mixture was stirred for 2 h at 0° C. under nitrogen atmosphere. The residue was purified by silica gel column chromatography, eluted with PE/THF (2:1) to afford tert-butyl (2S)-2-({1-cyano-2-[2,8-difluoro-9-(oxetan-3-yl)-6H-benzo[c]chromen-3-yl]ethyl}carbamoyl)-1,4-oxazepane-4-carboxylate (900 mg, 90.1%) as a colorless oil. LCMS (ES, m/z): [M+H]⁺: 570.

### Step 19. Synthesis of (2S)—N-{1-cyano-2-[2,8-difluoro-9-(oxetan-3-yl)-6H-benzo[c]chromen-3-yl]ethyl}-1,4-oxazepane-2-carboxamide

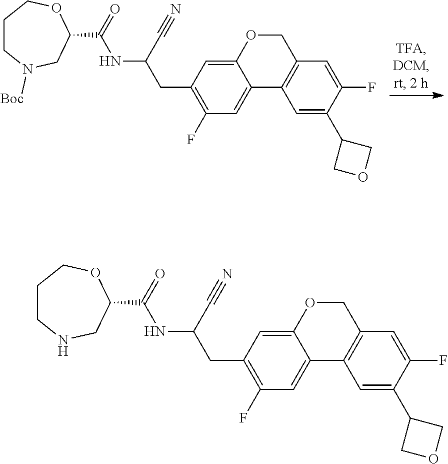 [SMILES: CC(C)(C)OC(=O)N1CCCO[C@H](C(=O)NC(C#N)Cc2cc3c(cc2F)-c2cc(C4COC4)c(F)cc2CO3)C1 ; N#CC(Cc1cc2c(cc1F)-c1cc(C3COC3)c(F)cc1CO2)NC(=O)[C@@H]1CNCCCO1]

To a stirred solution of tert-butyl (2S)-2-({1-cyano-2-[2,8-difluoro-9-(oxetan-3-yl)-6H-benzo[c]chromen-3-yl]ethyl}carbamoyl)-1,4-oxazepane-4-carboxylate (800 mg, 1.404 mmol, 1.0 equiv) in DCM (15 mL) was added TFA (1.5 mL). The resulting mixture was stirred for 2 h at room temperature. The mixture was basified to pH 8 with saturated NaHCO₃ (aq.). The resulting mixture was extracted with CH₂Cl₂ (3×20 mL). The combined organic layers were washed with brine (20 mL), dried over anhydrous Na₂SO₄. After filtration, the filtrate was concentrated under reduced pressure. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound B2 (300 mg, 45.5%) as a white solid. LCMS (ES, m/z): [M+H]⁺: 470. ¹H NMR (300 MHz, DMSO-d6) δ 8.68 (t, J=8.8 Hz, 1H), 8.02-7.89 (m, 2H), 7.23-7.13 (m, 1H), 6.99 (dd, J=6.6, 1.6 Hz, 1H), 5.17-5.03 (m, 2H), 5.09-4.87 (m, 3H), 4.85 (dd, J=7.4, 5.8 Hz, 2H), 4.62-4.45 (m, 1H), 4.05-3.80 (m, 2H), 3.72 (dtd, J=12.1, 7.7, 4.2 Hz, 1H), 3.31-2.98 (m, 3H), 2.89-2.52 (m, 3H), 1.82-1.62 (m, 2H).

### Example 44: Synthesis of (2S)—N-[(1S)-1-cyano-2-{13-fluoro-9-methyl-8-oxo-9-azatricyclo[9.4.0.0\hat{ }{2,7}1]pentadeca-1(11),2,4,6,12,14-hexaen-5-yl}ethyl]-1,4-oxazepane-2-carboxamide (Compound B3)

### Step 1. Synthesis of 4-fluoro-2-[(methylamino)methyl]phenylboronic acid

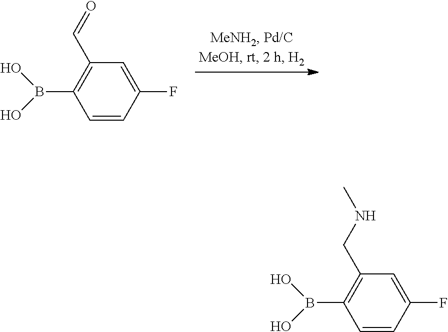

To a stirred solution of 4-fluoro-2-formylphenylboronic acid (5 g, 29.77 mmol, 1.0 equiv) in MeOH (70 mL) was added methylamine (45 mL, 89.32 mmol, 3.0 equiv) (2 M in methanol). The resulting mixture was stirred for 2 h at room temperature. To the above mixture was added Pd/C (1.5 g). The resulting mixture was stirred for 2 h at room temperature under hydrogen (5 atm) atmosphere. The resulting mixture was filtered, the filter cake was washed with MeOH (3×50 mL). The filtrate was concentrated under reduced pressure. This resulted in 4-fluoro-2-[(methylamino)methyl]phenylboronic acid (4 g, 73.4%) as white solid. LCMS (ES) [M+1]⁺ m/z: 184.

### Step 2. Synthesis of 5-bromo-13-fluoro-9-methyl-9-azatricyclo[9.4.0.0\hat{ }{2,7}]pentadeca-1(11),2,4,6,12,14-hexaen-8-one

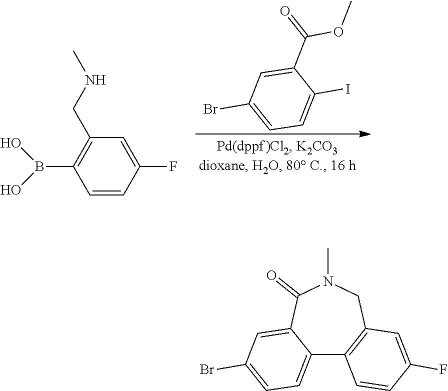 [SMILES: CNCc1cc(F)ccc1B(O)O ; COC(=O)c1cc(Br)ccc1I ; CN1Cc2cc(F)ccc2-c2ccc(Br)cc2C1=O]

To a stirred solution of 4-fluoro-2-[(methylamino)methyl]phenylboronic acid (4 g, 21.86 mmol, 1.0 equiv) and methyl 5-bromo-2-iodobenzoate (7.45 g, 21.86 mmol, 1.0 equiv) in dioxane (50 mL) and H₂O (5 mL) were added K₂CO₃ (6.04 g, 43.72 mmol, 2.0 equiv) and Pd(dppf)Cl₂ (1.60 g, 2.19 mmol, 0.1 equiv). The resulting mixture was stirred for 16 h at 80° C. under nitrogen atmosphere. The mixture was allowed to cool down to room temperature. The resulting mixture was concentrated under reduced pressure. The residue was purified by silica gel column chromatography, eluted with PE/THF (7:1) to afford 5-bromo-13-fluoro-9-methyl-9-azatricyclo[9.4.0.0\hat{ }{2,7}]pentadeca-1(11),2,4,6,12,14-hexaen-8-one (0.8 g, 11.4%) as light yellow solid. LCMS (ES) [M+1]⁺ m/z: 320.

### Step 3. Synthesis of methyl (2S)-2-[(tert-butoxycarbonyl)amino]-3-{13-fluoro-9-methyl-8-oxo-9-azatricyclo[9.4.0.0\hat{ }{2,7}]pentadeca-1(11),2,4,6,12,14-hexaen-5-yl}propanoate

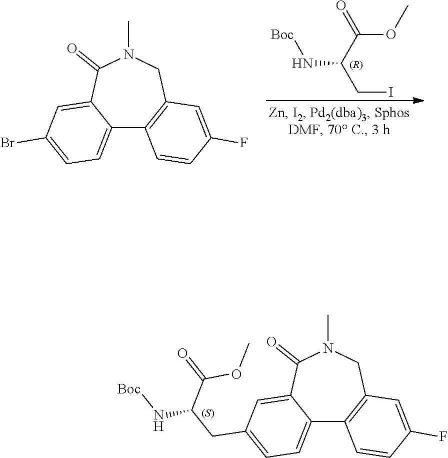

To a stirred mixture of Zn (490 mg, 7.50 mmol, 3.0 equiv) in DMF (12 mL) were added I₂ (95 mg, 0.38 mmol, 0.15 equiv) and methyl (2R)-2-[(tert-butoxycarbonyl)amino]-3-iodopropanoate (822 mg, 2.50 mmol, 1.0 equiv) at room temperature under nitrogen atmosphere. To the above mixture was added I₂ (95 mg, 0.38 mmol, 0.15 equiv), 5-bromo-13-fluoro-9-methyl-9-azatricyclo[9.4.0.0\hat{ }{2,7}]pentadeca-1(11),2,4,6,12,14-hexaen-8-one (800 mg, 2.50 mmol, 1.0 equiv), Pd₂(dba)₃ (114 mg, 0.12 mmol, 0.05 equiv) and Sphos (102 mg, 0.25 mmol, 0.1 equiv). The resulting mixture was stirred for 3 h at 70° C. under nitrogen atmosphere. The mixture was allowed to cool down to room temperature. The resulting mixture was diluted with water (40 mL). The resulting mixture was extracted with EtOAc (3×20 mL). The combined organic layer was washed with brine (2×20 mL), dried over anhydrous Na₂SO₄. After filtration, the filtrate was concentrated under reduced pressure. The residue was purified by silica gel column chromatography, eluted with PE/THF (2:1) to afford methyl (2S)-2-[(tert-butoxycarbonyl)amino]-3-{13-fluoro-9-methyl-8-oxo-9-azatricyclo[9.4.0.0\hat{ }{2,7}]pentadeca-1(11),2,4,6,12,14-hexaen-5-yl}propanoate (800 mg, 72.3%) as light yellow solid. LCMS (ES) [M+1]⁺ m/z: 443.

### Step 4. Synthesis of tert-butyl N-[(1S)-1-carbamoyl-2-{13-fluoro-9-methyl-8-oxo-9-azatricyclo[9.4.0.0\hat{ }{2,7}]pentadeca-1(11),2,4,6,12,14-hexaen-5-yl}ethyl]carbamate

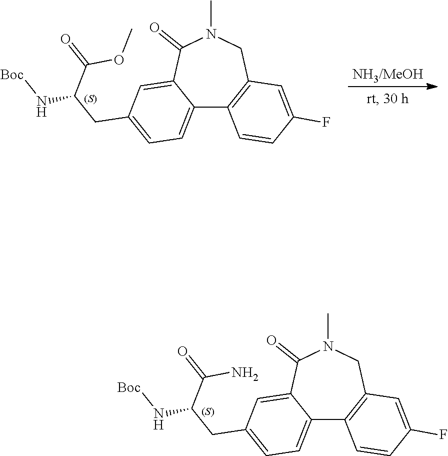

To a stirred solution of NH₃(g) in MeOH (30 mL) was added methyl (2S)-2-[(tert-butoxycarbonyl)amino]-3-{13-fluoro-9-methyl-8-oxo-9-azatricyclo[9.4.0.0\hat{ }{2,7}]pentadeca-1(11),2,4,6,12,14-hexaen-5-yl}propanoate (800 mg, 1.81 mmol, 1.0 equiv). The resulting mixture was stirred for 30 h at room temperature. The resulting mixture was concentrated under reduced pressure. This resulted in tert-butyl N-[(1S)-1-carbamoyl-2-{13-fluoro-9-methyl-8-oxo-9-azatricyclo[9.4.0.0\hat{ }{2,7}]pentadeca-1(11),2,4,6,12,14-hexaen-5-yl}ethyl]carbamate (700 mg, 90.5%) as light yellow solid. LCMS (ES) [M+1]⁺ m/z: 428.

### Step 5. Synthesis of tert-butyl N-[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]carbamate

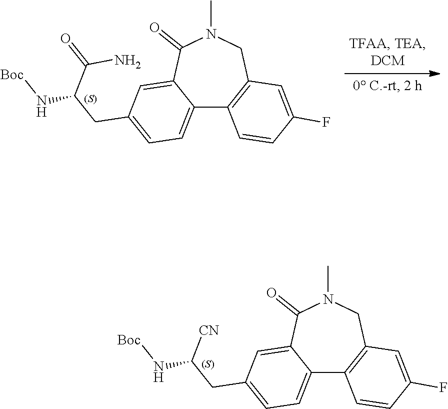

To a stirred solution of tert-butyl N-[(1S)-1-carbamoyl-2-{13-fluoro-9-methyl-8-oxo-9-azatricyclo[9.4.0.0\hat{ }{2,7}]pentadeca-1(11),2,4,6,12,14-hexaen-5-yl}ethyl]carbamate (700 mg, 1.64 mmol, 1.0 equiv) and TEA (662 mg, 6.55 mmol, 4.0 equiv) in DCM (15 mL) was added TFAA (687 mg, 3.28 mmol, 2.0 equiv) dropwise at 0° C. under nitrogen atmosphere. The resulting mixture was stirred for 2 h at room temperature. The resulting mixture was diluted with water (30 mL), extracted with CH₂Cl₂ (3×20 mL). The combined organic layer was washed with brine (20 mL), dried over anhydrous Na₂SO₄. After filtration, the filtrate was concentrated under reduced pressure. This resulted in tert-butyl N-[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]carbamate (600 mg, 90%) as light yellow solid. LCMS (ES) [M+1]⁺ m/z: 410.

### Step 6. Synthesis of (2S)-2-amino-3-{13-fluoro-9-methyl-8-oxo-9-azatricyclo[9.4.0.0\hat{ }{2,7}]pentadeca-1(11),2,4,6,12,14-hexaen-5-yl}propanenitrile

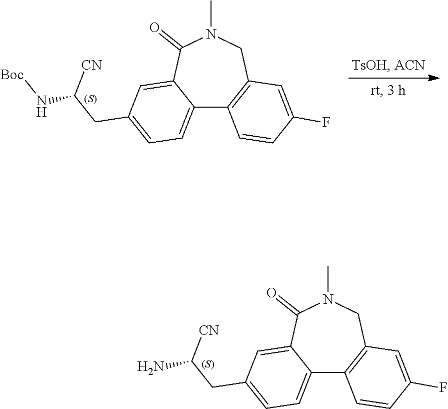

To a stirred solution tert-butyl N-[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]carbamate (600 mg, 1.46 mmol, 1.0 equiv) in ACN (12 mL) was added TsOH (757 mg, 4.40 mmol, 3.0 equiv). The resulting mixture was stirred for 3 h at room temperature. The reaction solution was purified by Prep-HPLC with the following conditions (Column, XBridge Prep C18 OBD Column, 19*150 mm, 5 um; mobile phase, Water (0.1% NH₃H₂O) and ACN (10% Phase B up to 80% in 20 min); Detector, UV 254 nm. This resulted in (2S)-2-amino-3-{13-fluoro-9-methyl-8-oxo-9-azatricyclo[9.4.0.0\hat{ }{2,7}]pentadeca-1(11),2,4,6,12,14-hexaen-5-yl}propanenitrile (350 mg, 77.2%) as white solid. LCMS (ES) [M+1]⁺ m/z: 310.

### Step 7. Synthesis of tert-butyl (2S)-2-{[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]carbamoyl}-1,4-oxazepane-4-carboxylate

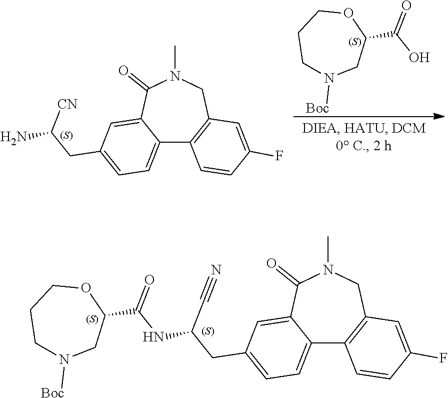

To a stirred solution of (2S)-2-amino-3-{13-fluoro-9-methyl-8-oxo-9-azatricyclo[9.4.0.0\hat{ }{2,7}]pentadeca-1(11),2,4,6,12,14-hexaen-5-yl}propanenitrile (97 mg, 0.31 mmol, 1.1 equiv) and (2S)-4-(tert-butoxycarbonyl)-1,4-oxazepane-2-carboxylic acid (70 mg, 0.28 mmol, 1.0 equiv) in DCM (2 mL) were added DIEA (110 mg, 0.86 mmol, 3.0 equiv) and HATU (130 mg, 0.34 mmol, 1.2 equiv) in portions at 0° C. under nitrogen atmosphere. The resulting mixture was stirred for 2 h at 0° C. Concentrated under reduced pressure to remove the solvent, the residue was purified by silica gel column chromatography, eluted with PE/THF (1:1) to afford tert-butyl (2S)-2-{[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]carbamoyl}-1,4-oxazepane-4-carboxylate (110 mg, 71.8%) as white solid. LCMS (ES) [M+1]⁺ m/z: 537.

### Step 8. Synthesis of (2S)—N-[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]-1,4-oxazepane-2-carboxamide

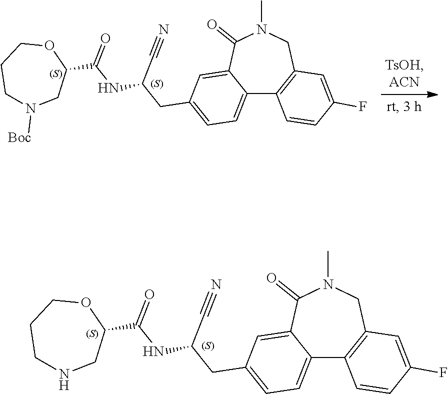

To a stirred solution of tert-butyl (2S)-2-{[(1S)-1-cyano-2-{13-fluoro-9-methyl-8-oxo-9-azatricyclo[9.4.0.0\hat{ }{2,7}]pentadeca-1(11),2,4,6,12,14-hexaen-5-yl}ethyl]carbamoyl}-1,4-oxazepane-4-carboxylate (100 mg, 0.18 mmol, 1.0 equiv) in ACN (2 mL) was added TsOH (96 mg, 0.56 mmol, 3.0 equiv). The resulting mixture was stirred for 3 h at room temperature. The reaction solution was purified by Prep-HPLC with the following conditions: Column, XBridge Prep C18 OBD Column, 19*150 mm, 5 um; mobile phase, Water (0.1% NH₃H₂O) and ACN (10% Phase B up to 80% in 20 min); Detector, UV 254 nm. This resulted in Compound B3 (30 mg, 36.8%) as white solid. LCMS (ES) [M+1]⁺ m/z: 437.1. ¹H NMR (300 MHz, DMSO-d₆) δ 8.62 (t, J=8.4 Hz, 1H), 7.74-7.69 (m, 2H), 7.62-7.51 (m, 2H), 7.48 (dd, J=9.0, 2.7 Hz, 1H), 7.40-7.28 (m, 1H), 5.04-4.93 (m, 1H), 4.24-4.13 (m, 2H), 3.93-3.83 (m, 2H), 3.74-3.65 (m, 1H), 3.27 (d, J=8.1 Hz, 2H), 3.15-3.08 (m, 1H), 3.07 (s, 3H), 2.87-2.77 (m, 1H), 2.77-2.57 (m, 2H), 1.86-1.57 (m, 2H).

### Example 45: Synthesis of (2S)—N-(1-cyano-2-{8-cyano-4-fluoro-6H-benzo[c]chromen-3-yl}ethyl)-1,4-oxazepane-2-carboxamide (Compound B4)

### Step 1. Synthesis of methyl 3-[(2-bromo-5-cyanophenyl)methoxy]-2-fluorobenzoate

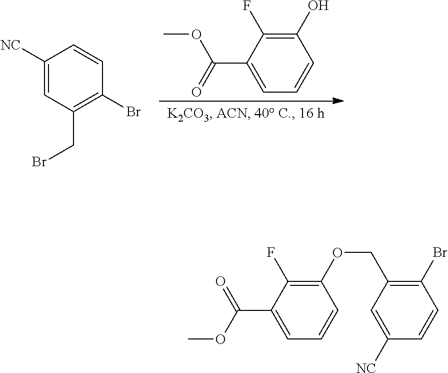 [SMILES: N#Cc1ccc(Br)c(CBr)c1 ; COC(=O)c1cccc(O)c1F ; COC(=O)c1cccc(OCc2cc(C#N)ccc2Br)c1F]

To a solution of 4-bromo-3-(bromomethyl)benzonitrile (8.5 g, 30.91 mmol, 1.0 equiv) and methyl 2-fluoro-3-hydroxybenzoate (5.26 g, 30.91 mmol, 1.0 equiv) in acetone (70 mL) was added K₂CO₃ (8.5 g, 61.82 mmol, 2.0 equiv). The mixture was stirred for 16 h at 40° C. The resulting mixture was diluted with H₂O (50 mL). The precipitated solid was collected by filtration and washed with H₂O (3×20 mL). This resulted in methyl 3-[(2-bromo-5-cyanophenyl)methoxy]-2-fluorobenzoate (11 g, 97.7%) as an off-white solid. ¹H NMR (400 MHz, DMSO-d₆) δ 8.07 (d, J=2.1 Hz, 1H), 7.96 (d, J=8.3 Hz, 1H), 7.83 (dd, J=8.3, 2.1 Hz, 1H), 7.58 (td, J=8.1, 1.6 Hz, 1H), 7.47 (ddd, J=7.9, 6.1, 1.6 Hz, 1H), 7.29 (td, J=8.1, 1.4 Hz, 1H), 5.27 (s, 2H), 3.86 (s, 3H).

### Step 2. Synthesis of methyl 8-cyano-4-fluoro-6H-benzo[c]chromene-3-carboxylate

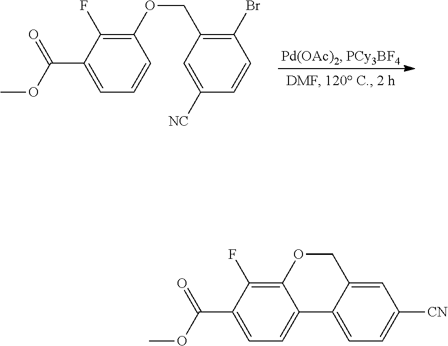 [SMILES: COC(=O)c1cccc(OCc2cc(C#N)ccc2Br)c1F ; COC(=O)c1ccc2c(c1F)OCc1cc(C#N)ccc1-2]

To a mixture of methyl 3-[(2-bromo-5-cyanophenyl)methoxy]-2-fluorobenzoate (11 g, 30.21 mmol, 1.0 equiv), K₂CO₃ (8.35 g, 60.41 mmol, 2.0 equiv) and PCy₃HBF₄ (1.1 g, 3.02 mmol, 0.1 equiv) in DMF (90 mL) was added Pd(OAc)₂ (338 mg, 1.51 mmol, 0.05 equiv) under nitrogen gas atmosphere. The mixture was stirred for 2 h at 120° C. under nitrogen atmosphere. The reaction was cooled to room temperature, diluted with water (100 mL). The resulting mixture was extracted with EtOAc (3×300 mL). The combined organic layer was washed with brine (3×500 mL), dried over anhydrous Na₂SO₄. After filtration, the filtrate was concentrated under reduced pressure. This resulted in methyl 8-cyano-4-fluoro-6H-benzo[c]chromene-3-carboxylate (6 g, 70.1%) as a black solid. ¹H NMR (300 MHz, DMSO-d₆) δ 8.11 (d, J=8.1 Hz, 1H), 7.98-7.88 (m, 2H), 7.86 (s, 1H), 7.55 (dd, J=8.4, 6.6 Hz, 1H), 5.33 (s, 2H), 3.88 (s, 3H).

### Step 3. Synthesis of 4-fluoro-3-(hydroxymethyl)-6H-benzo[c]chromene-8-carbonitrile

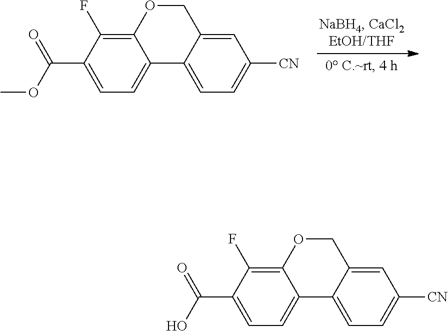 [SMILES: COC(=O)c1ccc2c(c1F)OCc1cc(C#N)ccc1-2 ; N#Cc1ccc2c(c1)COc1c-2ccc(C(=O)O)c1F]

To a stirred solution of methyl 8-cyano-4-fluoro-6H-benzo[c]chromene-3-carboxylate (6 g, 21.18 mmol, 1.0 equiv) in mixed solvent EtOH/THF (1 v/1 v) (100 mL) was added CaCl₂ (0.47 g, 4.23 mmol, 0.2 equiv) and NaBH₄ (3.21 g, 84.78 mmol, 4.0 equiv) at 0° C. The resulting mixture was stirred for additional 4 h at room temperature. The reaction was quenched with water (200 mL), extracted with EtOAc (3×100 mL). The combined organic layer was washed with brine (3×100 mL), dried over anhydrous Na₂SO₄. After filtration, the filtrate was concentrated under reduced pressure. This resulted in 4-fluoro-3-(hydroxymethyl)-6H-benzo[c]chromene-8-carbonitrile (3 g, 55.5%) as a light yellow solid. ¹H NMR (400 MHz, DMSO-d₆) δ 8.04 (d, J=8.1 Hz, 1H), 7.92-7.85 (m, 1H), 7.82 (s, 1H), 7.81-7.76 (m, 1H), 7.19 (t, J=7.4 Hz, 1H), 5.37 (t, J=5.7 Hz, 1H), 5.26 (s, 2H), 4.58 (d, J=6.0 Hz, 2H).

### Step 4. Synthesis of 3-(bromomethyl)-4-fluoro-6H-benzo[c]chromene-8-carbonitrile

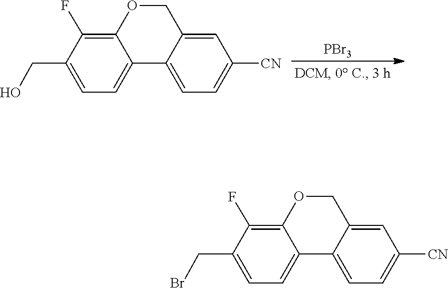 [SMILES: N#Cc1ccc2c(c1)COc1c-2ccc(CO)c1F ; "PBr3" ; N#Cc1ccc2c(c1)COc1c-2ccc(CBr)c1F]

To a stirred solution of 4-fluoro-3-(hydroxymethyl)-6H-benzo[c]chromene-8-carbonitrile (1.4 g, 5.48 mmol, 1.0 equiv) in DCM (15 mL) was added phosphorus tribromide (0.74 g, 2.74 mmol, 0.5 equiv) dropwise at 0° C. The resulting mixture was stirred for additional 3 h at 0° C. The reaction was quenched with water (20 mL), extracted with CH₂Cl₂ (3×30 mL). The combined organic layer was washed with brine (3×30 mL), dried over anhydrous Na₂SO₄. After filtration, the filtrate was concentrated under reduced pressure to afford 3-(bromomethyl)-4-fluoro-6H-benzo[c]chromene-8-carbonitrile (1.4 g, 80.2%) as a yellow solid and used to the next step directly without further purification. ¹H NMR (300 MHz, DMSO-d₆) δ 8.07 (d, J=8.2 Hz, 1H), 7.91 (d, J=8.2 Hz, 1H), 7.88-7.77 (m, 2H), 7.26 (t, J=7.6 Hz, 1H), 5.30 (s, 2H), 4.74 (s, 2H).

### Step 5. Synthesis of 3-{2-cyano-2-[(diphenylmethylidene)amino]ethyl}-4-fluoro-6H-benzo[c]chromene-8-carbonitrile

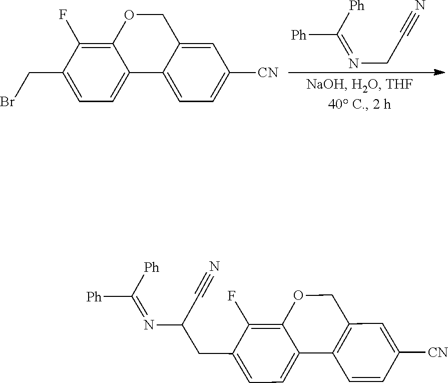 [SMILES: N#Cc1ccc2c(c1)COc1c-2ccc(CBr)c1F ; N#CCN=C(c1ccccc1)c1ccccc1 ; N#Cc1ccc2c(c1)COc1c-2ccc(CC(C#N)N=C(c2ccccc2)c2ccccc2)c1F]

A solution of 3-(bromomethyl)-4-fluoro-6H-benzo[c]chromene-8-carbonitrile (1.4 g, 4.40 mmol, 1.0 equiv) in THF (15 mL) was added 2-[(diphenylmethylidene)amino]acetonitrile (0.97 g, 4.40 mmol, 1.0 equiv), benzyltrimethylazanium chloride (81 mg, 0.44 mmol, 0.1 equiv), NaOH (352 mg, 8.80 mmol, 2.0 equiv) in H₂O (1.5 mL) was stirred for 2 h at 40° C. The resulting mixture was extracted with EtOAc (3×50 mL). The combined organic layer was washed with brine (3×100 mL), dried over anhydrous Na₂SO₄. After filtration, the filtrate was concentrated under reduced pressure. The residue was purified by silica gel column chromatography, eluted with PE/THF (5:1) to afford 3-{2-cyano-2-[(diphenylmethylidene)amino]ethyl}-4-fluoro-6H-benzo[c]chromene-8-carbonitrile (1.1 g, 54.64%) as a light yellow solid. LCMS (ES, m/z): [M+H]⁺: 458.

### Step 6. Synthesis of 3-(2-amino-2-cyanoethyl)-4-fluoro-6H-benzo[c]chromene-8-carbonitrile

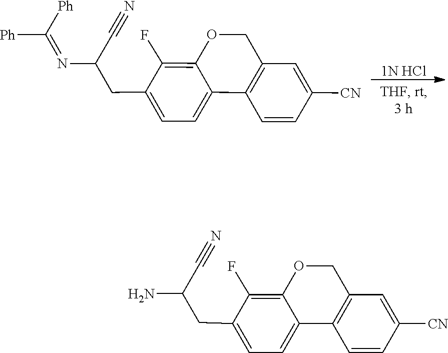

Into a 100 mL round-bottom flask were added 3-{2-cyano-2-[(diphenylmethylidene)amino]ethyl}-4-fluoro-6H-benzo[c]chromene-8-carbonitrile (1 g, 2.19 mmol, 1.0 equiv) and HCl (1 M) (2.5 mL), THF (50 mL), H₂O (5 mL) at room temperature. The resulting mixture was stirred for 3 h at room temperature. The mixture was basified to pH 12 with K₂CO₃(aq). The resulting mixture was extracted with EtOAc (3×30 mL). The combined organic layer was washed with brine (3×10 mL), dried over anhydrous Na₂SO₄. After filtration, the filtrate was concentrated under reduced pressure. The residue was purified by silica gel column chromatography, eluted with PE/THF (1:2) to afford 3-(2-amino-2-cyanoethyl)-4-fluoro-6H-benzo[c]chromene-8-carbonitrile (500 mg, 78%) as a yellow solid. LCMS (ES, m/z): [M+H]⁺: 294.

### Step 7. Synthesis of tert-butyl (2S)-2-[(1-cyano-2-{8-cyano-4-fluoro-6H-benzo[c]chromen-3-yl}ethyl)carbamoyl]-1,4-oxazepane-4-carboxylate

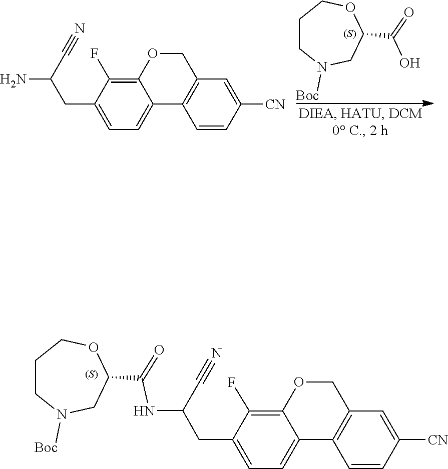

To a stirred solution of 3-(2-amino-2-cyanoethyl)-4-fluoro-6H-benzo[c]chromene-8-carbonitrile (115 mg, 0.39 mmol, 1.0 equiv) and (2S)-4-(tert-butoxycarbonyl)-1,4-oxazepane-2-carboxylic acid (96 mg, 0.39 mmol, 1.0 equiv), DIEA (152 mg, 1.17 mmol, 3.0 equiv) in DMF (5 mL) were added HATU (178 mg, 0.47 mmol, 1.2 equiv) in portions at 0° C. The resulting mixture was stirred for additional 2 h at 0° C. The reaction was quenched with water (15 mL), extracted with EtOAc (2×20 mL). The combined organic layer was washed with brine (3×10 mL), dried over anhydrous Na₂SO₄. After filtration, the filtrate was concentrated under reduced pressure. The residue was purified by silica gel column chromatography, eluted with PE/THF (1:1) to afford tert-butyl (2S)-2-[(1-cyano-2-{8-cyano-4-fluoro-6H-benzo[c]chromen-3-yl}ethyl)carbamoyl]-1,4-oxazepane-4-carboxylate (130 mg, 63.7%) as a light yellow oil. LCMS (ES, m/z): [M+H]⁺: 521.

### Step 8. Synthesis of (2S)—N-(1-cyano-2-{8-cyano-4-fluoro-6H-benzo[c]chromen-3-yl}ethyl)-1,4-oxazepane-2-carboxamide

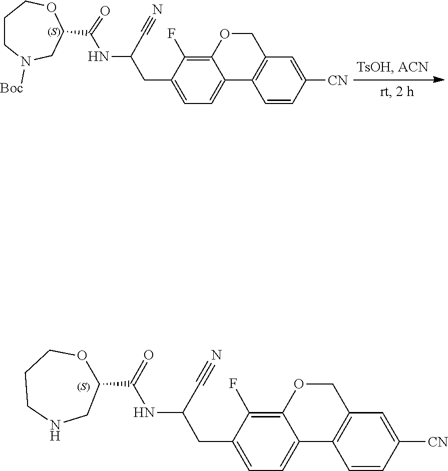

Into a 25 mL round-bottom flask were added tert-butyl (2S)-2-[(1-cyano-2-{8-cyano-4-fluoro-6H-benzo[c]chromen-3-yl}ethyl)carbamoyl]-1,4-oxazepane-4-carboxylate (130 mg, 0.25 mmol, 1.0 equiv), TsOH·H₂O (142 mg, 0.75 mmol, 3.0 equiv) and ACN (4 mL) at room temperature. The resulting mixture was stirred for additional 2 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound B4 (20 mg, 19.0%) as a white solid. LCMS (ES, m/z): [M+H]⁺: 421.1. ¹H NMR (400 MHz, DMSO-d₆): δ 8.71-8.67 (m, 1H), 8.05 (dd, J=8.3, 2.5 Hz, 1H), 7.89 (d, J=8.1 Hz, 1H), 7.82 (s, 1H), 7.78 (dd, J=8.4, 2.8 Hz, 1H), 7.12-7.06 (m, 1H), 5.31-5.23 (m, 2H), 5.10-4.97 (m, 1H), 4.01-3.92 (m, 1H), 3.90-3.83 (m, 1H), 3.76-3.67 (m, 1H), 3.30-3.17 (m, 2H), 3.16-3.01 (m, 1H), 2.86-2.53 (m, 4H), 1.88-1.58 (m, 2H).

### Example 46: Synthesis of (2S)—N-(1-cyano-2-(8-cyano-2-fluoro-6H-benzo[c]chromen-3-yl)ethyl)-1,4-oxazocane-2-carboxamide (Compound B7), (2S)—N-[(1S)-1-cyano-2-{8-cyano-2-fluoro-6H-benzo[c]chromen-3-yl}ethyl]-1,4-oxazocane-2-carboxamide (Compound B8) and (2S)—N-[(1R)-1-cyano-2-{8-cyano-2-fluoro-6H-benzo[c]chromen-3-yl}ethyl]-1,4-oxazocane-2-carboxamide (Compound B9)

### Step 1: Synthesis of 4-bromo-3-(bromomethyl)benzonitrile

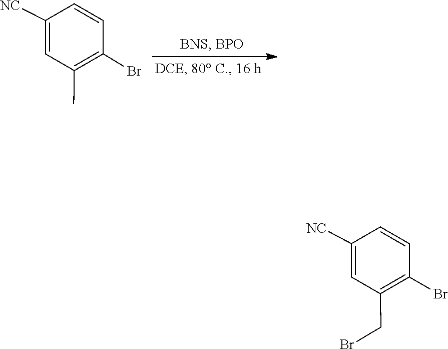

A solution of 4-bromo-3-methylbenzonitrile (8 g, 40.81 mmol, 1.0 equiv), NBS (8.72 g, 48.97 mmol, 1.2 equiv) and BPO (3.14 g, 12.24 mmol, 0.3 equiv) in DCE (100 mL) was stirred for 16 h at 80° C. under nitrogen atmosphere. The reaction was cooled to room temperature, diluted with water (100 mL), extracted with CH₂Cl₂ (2×80 mL). The combined organic layer was washed with brine (2×80 mL), dried over anhydrous Na₂SO₄. After filtration, the filtrate was concentrated under reduced pressure. The residue was purified by silica gel column chromatography, eluted with PE/EA (5:1) to afford 4-bromo-3-(bromomethyl) benzo nitrile (6.5 g, 58%) as a white solid.

### Step 2: Synthesis of (S)—N—((S)-1-cyano-2-(4-(3-oxoisoindolin-5-yl) phenyl) ethyl)-1,4-oxazepane-2-carboxamide

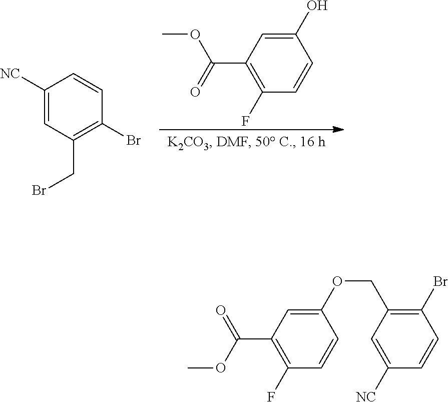 [SMILES: N#Cc1ccc(Br)c(CBr)c1 ; COC(=O)c1cc(O)ccc1F ; COC(=O)c1cc(OCc2cc(C#N)ccc2Br)ccc1F]

A mixture of 4-bromo-3-(bromomethyl) benzo nitrile (6.5 g, 23.64 mmol, 1.0 equiv), K₂CO₃ (9.80 g, 70.92 mmol, 3.0 equiv) and 4-bromo-3-(bromomethyl) benzo nitrile (6.5 g, 23.64 mmol, 1.0 equiv) in DMF (70 mL) was stirred for 16 h at 50° C. The resulting mixture was diluted with water (300 mL). The precipitated solid was collected by filtration and washed with water (2×80 mL), dried under infrared lamp for 5 h. This resulted in methyl 5-[(2-bromo-5-cyanophenyl) methoxy]-2-fluorobenzoate (3.7 g, 43%) as light-yellow solid.

### Step 3: Synthesis of methyl 8-cyano-2-fluoro-6H-benzo[c]chromene-3-carboxylate

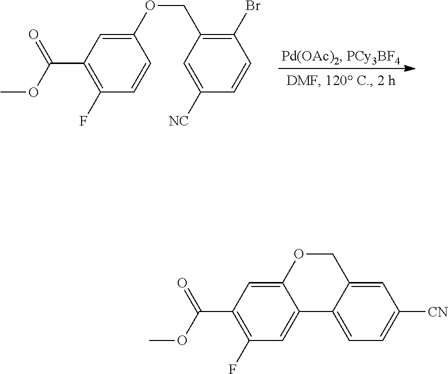 [SMILES: COC(=O)c1cc(OCc2cc(C#N)ccc2Br)ccc1F ; COC(=O)c1cc2c(cc1F)-c1ccc(C#N)cc1CO2]

To a stirred solution of methyl 5-[(2-bromo-5-cyanophenyl) methoxy]-2-fluorobenzoate (3.7 g, 10.16 mmol, 1.0 equiv), PCy₃HBF₄ (0.37 g, 1.02 mmol, 0.1 equiv) and K₂CO₃ (4.21 g, 30.48 mmol, 3.0 equiv) in DMF (40 mL) was added Pd(OAc)₂ (0.23 g, 1.02 mmol, 0.1 equiv) in portions at room temperature under nitrogen atmosphere. The resulting mixture was stirred for 2 h at 120° C. under nitrogen atmosphere. The reaction was cooled to room temperature, diluted with water (100 mL). The resulting mixture was extracted with EtOAc (4×100 mL). The combined organic layer was washed with water (3×100 mL), dried over anhydrous Na₂SO₄. After filtration, the filtrate was concentrated under reduced pressure. The residue was purified by silica gel column chromatography, eluted with PE/EA (3:1) to afford methyl 8-cyano-2-fluoro-6H-benzo[c]chromene-3-carboxylate (1.7 g, 59%) as light-yellow solid.

### Step 4: Synthesis of 2-fluoro-3-(hydroxymethyl)-6H-benzo[c]chromene-8-carbonitrile

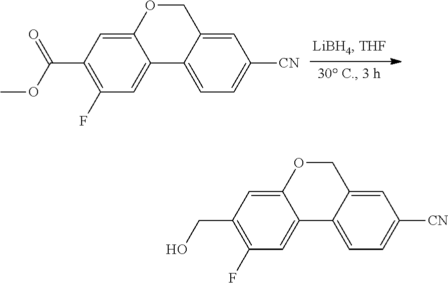 [SMILES: COC(=O)c1cc2c(cc1F)-c1ccc(C#N)cc1CO2 ; N#Cc1ccc2c(c1)COc1cc(CO)c(F)cc1-2]

To a stirred solution of methyl 8-cyano-2-fluoro-6H-benzo[c]chromene-3-carboxylate (1.7 g, 6.00 mmol, 1.0 equiv) in THF (20 mL) was added LiBH₄ (0.14 g, 6.60 mmol, 1.1 equiv) dropwise at 0° C. under nitrogen atmosphere. The resulting mixture was stirred for 3 h at 30° C. under nitrogen atmosphere. The resulting mixture was extracted with CH₂Cl₂ (3×20 mL). The combined organic layer was washed with water (2×20 mL), dried over anhydrous Na₂SO₄. After filtration, the filtrate was concentrated under reduced pressure. The residue was purified by silica gel column chromatography, eluted with PE/EA (4:1) to afford 2-fluoro-3-(hydroxymethyl)-6H-benzo[c]chromene-8-carbonitrile (750 mg, 49%) as a white solid. LCMS (ES) [M-H₂O+1]⁺ m/z: 238.

### Step 5: Synthesis of 3-(bromomethyl)-2-fluoro-6H-benzo[c]chromene-8-carbonitrile

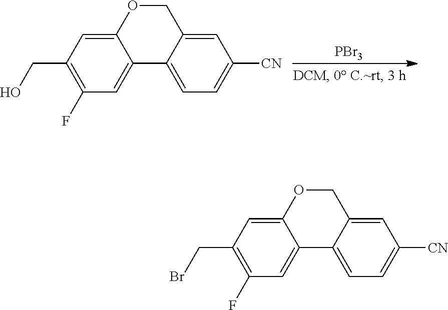 [SMILES: N#Cc1ccc2c(c1)COc1cc(CO)c(F)cc1-2 ; "PBr3" ; N#Cc1ccc2c(c1)COc1cc(CBr)c(F)cc1-2]

To a stirred solution of 2-fluoro-3-(hydroxymethyl)-6H-benzo[c]chromene-8-carbonitrile (750 mg, 2.94 mmol, 1.0 equiv) in DCM (10 mL) was added phosphorus tribromide (795 mg, 2.94 mmol, 1.0 equiv) dropwise at 0° C. The resulting mixture was stirred for 3 h at room temperature. The resulting mixture was diluted with water 10 mL, extracted with CH₂Cl₂ (3×20 mL). The combined organic layer was washed with water (3×10 mL), dried over anhydrous Na₂SO₄. After filtration, the filtrate was concentrated under reduced pressure. This resulted in 3-(bromomethyl)-2-fluoro-6H-benzo[c]chromene-8-carbonitrile (580 mg, 62%) as white solid.

### Step 6: Synthesis of 3-{2-cyano-2-[(diphenylmethylidene)amino]ethyl}-2-fluoro-6H-benzo[c]chromene-8-carbonitrile

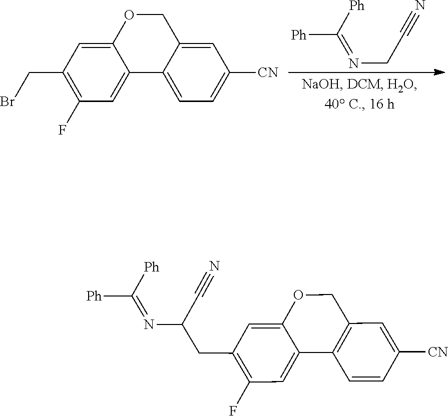 [SMILES: N#Cc1ccc2c(c1)COc1cc(CBr)c(F)cc1-2 ; N#CCN=C(c1ccccc1)c1ccccc1 ; N#Cc1ccc2c(c1)COc1cc(CC(C#N)N=C(c3ccccc3)c3ccccc3)c(F)cc1-2]

To a stirred solution of 3-(bromomethyl)-2-fluoro-6H-benzo[c]chromene-8-carbonitrile (580 mg, 1.82 mmol, 1.0 equiv) and 2-[(diphenylmethylidene)amino]acetonitrile (401 mg, 1.82 mmol, 1.0 equiv) in DCM (10 mL) and H₂O (1 mL) was added NaOH (146 mg, 3.65 mmol, 2.0 equiv) in portions at 0° C. The resulting mixture was stirred for 16 h at 40° C. The resulting mixture was diluted with water (20 mL), extracted with DCM (2×20 mL). The combined organic layer was washed with water (2×10 mL), dried over anhydrous Na₂SO₄. After filtration, the filtrate was concentrated under reduced pressure. The residue was purified by silica gel column chromatography, eluted with PE/EA (3:1) to afford 3-{2-cyano-2-[(diphenylmethylidene)amino]ethyl}-2-fluoro-6H-benzo[c]chromene-8-carbonitrile (400 mg, 48%) as white solid. LCMS (ES) [M+1]⁺ m/z: 458.

### Step 7: Synthesis of 3-(2-amino-2-cyanoethyl)-2-fluoro-6H-benzo[c]chromene-8-carbonitrile

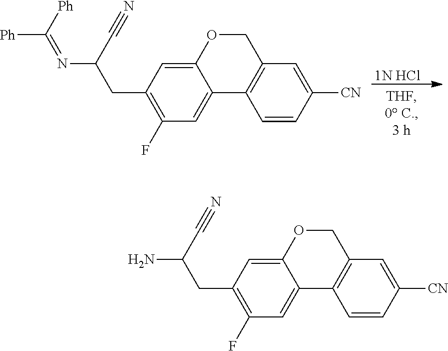

To a stirred solution of 3-{2-cyano-2-[(diphenylmethylidene)amino]ethyl}-2-fluoro-6H-benzo[c]chromene-8-carbonitrile (400 mg, 0.87 mmol, 1.0 equiv) in tetrahydrofuran (4 mL) was added HCl (1M) (1 mL) dropwise at 0° C. The resulting mixture was stirred for 3 h at room temperature. The resulting mixture was diluted with water (20 mL). The resulting mixture was extracted with diethyl ether (3×10 mL). The aqueous phase was neutralized to pH 7 with saturated NaHCO₃ (aq.). The aqueous layer was extracted with EtOAc (3×20 mL). The combined organic layer was washed with water (2×10 mL), dried over anhydrous Na₂SO₄. After filtration, the filtrate was concentrated under reduced pressure. This resulted in 3-(2-amino-2-cyanoethyl)-2-fluoro-6H-benzo[c]chromene-8-carbonitrile (170 mg, 66.3%) as light-yellow solid. LCMS (ES) [M+1]⁺ m/z: 294.

### Step 8: Synthesis of tert-butyl (2S)-2-{[(1S)-1-cyano-2-{8-cyano-2-fluoro-6H-benzo[c]chromen-3-yl}ethyl]carbamoyl}-1,4-oxazocane-4-carboxylate

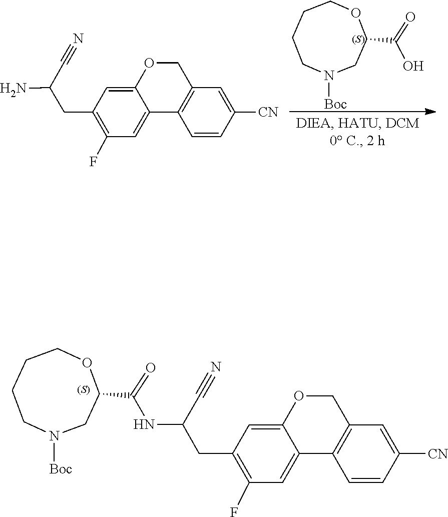

To a stirred solution of 3-(2-amino-2-cyanoethyl)-2-fluoro-6H-benzo[c]chromene-8-carbonitrile (70 mg, 0.24 mmol, 1.0 equiv) and DIEA (92 mg, 0.72 mmol, 3.0 equiv) in DCM (1 mL) was added HATU (108 mg, 0.29 mmol, 1.2 equiv) in portions at 0° C. The resulting mixture was stirred for 2 h at 0° C. The resulting mixture was concentrated under vacuum. The residue was purified by silica gel column chromatography, eluted with PE/EA (1:1) to afford tert-butyl (2S)-2-{[(1S)-1-cyano-2-{8-cyano-2-fluoro-6H-benzo[c]chromen-3-yl}ethyl]carbamoyl}-1,4-oxazocane-4-carboxylate (105 mg, 82%) as a white solid. LCMS (ES) [M+1]⁺ m/z: 535.

### Step 9: Synthesis of (2S)—N-[(1S)-1-cyano-2-{8-cyano-2-fluoro-6H-benzo[c]chromen-3-yl}ethyl]-1,4-oxazocane-2-carboxamide

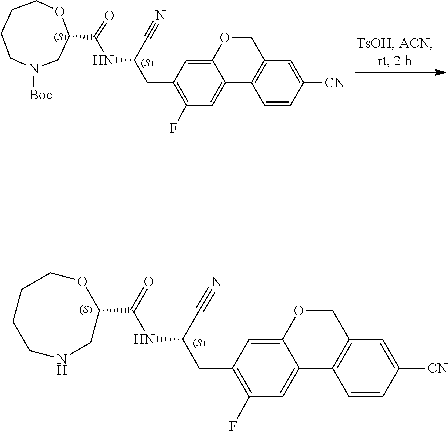

To a stirred solution of tert-butyl (2S)-2-{[(1S)-1-cyano-2-{8-cyano-2-fluoro-6H-benzo[c]chromen-3-yl}ethyl]carbamoyl}-1,4-oxazocane-4-carboxylate (100 mg, 0.19 mmol, 1.0 equiv) in ACN (1.5 mL) was added TsOH (112 mg, 0.65 mmol, 3.5 equiv) in one portion at room temperature. The resulting mixture was stirred for 2 h at room temperature. The reaction solution was purified by reversed-phase flash chromatography with the following conditions: column, C18 silica gel-120 g; mobile phase, MeCN in Water (0.1% NH₃·H₂O), 10% to 55% gradient in 10 min; detector, UV 254 nm. This resulted in Compound B7 (20 mg, 24%) as white solid. LCMS (ES) [M+1]⁺ m/z: 435. ¹H NMR (400 MHz, DMSO-d₆) δ 8.72 (dd, J=8.6, 4.8 Hz, 1H), 8.06 (dd, J=8.3, 2.5 Hz, 1H), 7.93-7.85 (m, 2H), 7.81 (d, J=1.6 Hz, 1H), 7.06-7.00 (m, 1H), 5.21-5.12 (m, 2H), 5.10-4.91 (m, 1H), 4.00-3.91 (m, 1H), 3.87-3.75 (m, 1H), 3.69-3.56 (m, 1H), 3.29-2.90 (m, 4H), 2.62-2.56 (m, 1H), 2.45-2.20 (m, 1H), 1.93-1.82 (m, 1H), 1.58-1.43 (m, 3H).

### Step 10: Synthesis of (2S)—N-[(1S)-1-cyano-2-{8-cyano-2-fluoro-6H-benzo[c]chromen-3-yl}ethyl]-1,4-oxazocane-2-carboxamide and (2S)—N-[(1R)-1-cyano-2-{8-cyano-2-fluoro-6H-benzo[c]chromen-3-yl}ethyl]-1,4-oxazocane-2-carboxamide

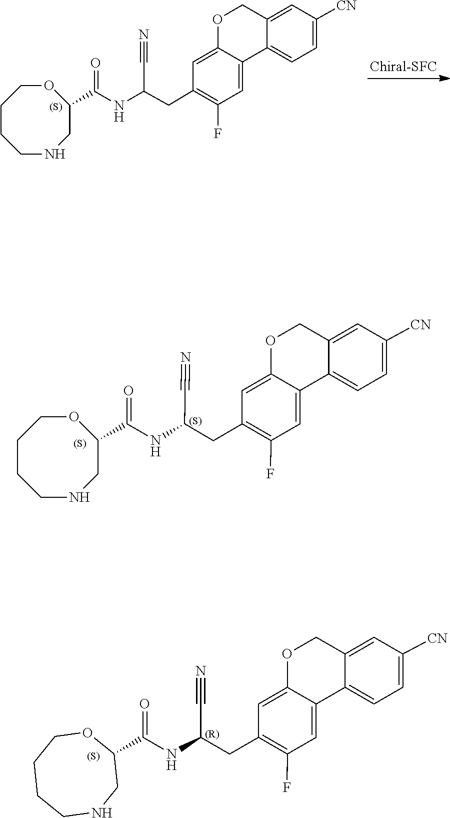

The product of (2S)—N-(1-cyano-2-{8-cyano-2-fluoro-6H-benzo[c]chromen-3-yl}ethyl)-1,4-oxazocane-2-carboxamide (80 mg, 0.18 mmol, 1.0 equiv) was purified by Prep-CHIRAL-SFC-150 with the following conditions: Column: XA-CHIRAL ART Cellulose-SB, 3*25 cm, 5 m; Mobile Phase A: CO₂, Mobile Phase B: MeOH (0.1% 2 M NH₃-MeOH); Flow rate: 80 mL/min; Gradient: isocratic 40% B; Column Temperature (° C.): 35; Back Pressure (bar): 100; Wave Length: 254 nm; RT1(min): 5.54; RT2(min): 6.22; Sample Solvent: MeOH; Injection Volume: 2.5 mL. This resulted in Compound B8 (25 mg, 31%) as white solid and Compound B9 (25 mg, 31%) as white solid. LCMS (ES) [M+1]⁺ m/z: 435.

Compound B8: LCMS (ES) [M+1]⁺ m/z: 435.0. ¹H NMR (300 MHz, DMSO-d₆) δ 8.73 (d, J=8.5 Hz, 1H), 8.06 (d, J=8.2 Hz, 1H), 7.94-7.84 (m, 2H), 7.80 (d, J=1.7 Hz, 1H), 7.04 (d, J=6.5 Hz, 1H), 5.17 (s, 2H), 5.03-4.89 (m, 1H), 4.00-3.92 (m, 1H), 3.77 (dd, J=9.8, 2.9 Hz, 1H), 3.63-3.57 (m, 1H), 3.28-3.18 (m, 2H), 3.08 (dd, J=13.9, 2.8 Hz, 1H), 3.02-2.94 (m, 1H), 2.61-2.55 (m, 1H), 2.41 (dd, J=13.9, 9.9 Hz, 1H), 1.94-1.83 (m, 1H), 1.60-1.45 (m, 3H).

Compound B9: LCMS (ES) [M+1]⁺ m/z: 435.0. ¹H NMR (300 MHz, DMSO-d₆) δ 8.71 (d, J=8.7 Hz, 1H), 8.05 (d, J=8.2 Hz, 1H), 7.95-7.84 (m, 2H), 7.80 (d, J=1.7 Hz, 1H), 7.01 (d, J=6.5 Hz, 1H), 5.16 (d, J=3.8 Hz, 2H), 5.13-4.97 (m, 1H), 3.99-3.91 (m, 1H), 3.85 (dd, J=9.7, 2.9 Hz, 1H), 3.69-3.62 (m, 1H), 3.32-3.08 (m, 2H), 3.01-2.87 (m, 2H), 2.62-2.55 (m, 1H), 2.23 (dd, J=14.0, 9.7 Hz, 1H), 1.92-1.76 (m, 1H), 1.57-1.46 (m, 3H).

### Example 47: Synthesis of (2S)—N-{1-cyano-2-[2,8-difluoro-9-(oxetan-3-yl)-6H-benzo[c]chromen-3-yl]ethyl}-1,4-oxazocane-2-carboxamide (Compound B10)

### Step 1. Synthesis of tert-butyl N-[(2S)-2,3-dihydroxypropyl]carbamate

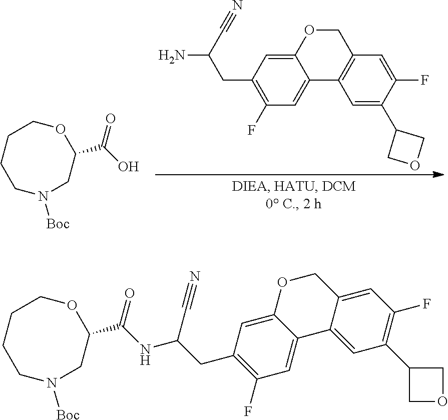 [SMILES: CC(C)(C)OC(=O)N1CCCCO[C@H](C(=O)O)C1 ; N#CC(N)Cc1cc2c(cc1F)-c1cc(C3COC3)c(F)cc1CO2 ; CC(C)(C)OC(=O)N1CCCCO[C@H](C(=O)NC(C#N)Cc2cc3c(cc2F)-c2cc(C4COC4)c(F)cc2CO3)C1]

To a stirred solution of (2S)-4-(tert-butoxycarbonyl)-1,4-oxazocane-2-carboxylic acid (80 mg, 0.309 mmol, 1.0 equiv) and 2-amino-3-[2,8-difluoro-9-(oxetan-3-yl)-6H-benzo[c]chromen-3-yl]propanenitrile (126 mg, 0.371 mmol, 1.2 equiv) in DCM (1.5 mL) were added DIEA (119 mg, 0.927 mmol, 3.0 equiv) and HATU (140 mg, 0.371 mmol, 1.2 equiv) in portions at 0° C. under nitrogen atmosphere. The resulting mixture was stirred at 0° C. for 2 h. The residue was purified by silica gel column chromatography, eluted with PE/THF (3:1) to afford tert-butyl (2S)-2-({1-cyano-2-[2,8-difluoro-9-(oxetan-3-yl)-6H-benzo[c]chromen-3-yl]ethyl}carbamoyl)-1,4-oxazocane-4-carboxylate (140 mg, 77.7%) as a white solid. LCMS (ES) [M+1]⁺ m/z: 584.

### Step 2. Synthesis of (2S)—N-{1-cyano-2-[2,8-difluoro-9-(oxetan-3-yl)-6H-benzo[c]chromen-3-yl]ethyl}-1,4-oxazocane-2-carboxamide

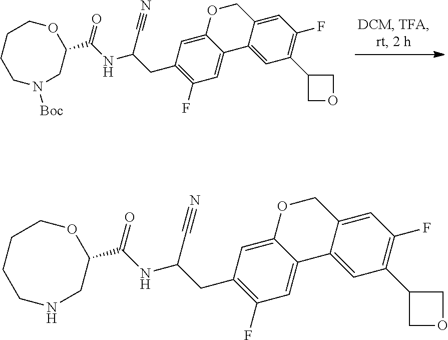 [SMILES: CC(C)(C)OC(=O)N1CCCCO[C@H](C(=O)NC(C#N)Cc2cc3c(cc2F)-c2cc(C4COC4)c(F)cc2CO3)C1 ; N#CC(Cc1cc2c(cc1F)-c1cc(C3COC3)c(F)cc1CO2)NC(=O)[C@@H]1CNCCCCO1]

To a stirred solution of tert-butyl (2S)-2-({1-cyano-2-[2,8-difluoro-9-(oxetan-3-yl)-6H-benzo[c]chromen-3-yl]ethyl}carbamoyl)-1,4-oxazocane-4-carboxylate (100 mg, 0.171 mmol, 1.0 equiv) in DCM (2 mL) was added TFA (0.2 mL). The resulting mixture was stirred at room temperature for 2 h. The residue was basified to pH 8 with saturated NaHCO₃ (aq.). The resulting mixture was extracted with CH₂Cl₂ (3×10 mL). The combined organic layers were washed with brine (10 mL), dried over anhydrous Na₂SO₄. After filtration, the filtrate was concentrated under reduced pressure. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound B10 (30 mg, 36.2%) as a white solid. LCMS (ES) [M+1]⁺ m/z: 484. ¹H NMR (300 MHz, DMSO-d6) δ 8.70 (dd, J=8.6, 3.3 Hz, 1H), 8.02-7.89 (m, 2H), 7.18 (d, J=10.2 Hz, 1H), 6.97 (t, J=6.9 Hz, 1H), 5.09 (d, J=3.2 Hz, 2H), 5.10-4.80 (m, 5H), 4.53 (p, J=8.0 Hz, 1H), 4.03-3.90 (m, 1H), 3.89-3.74 (m, 1H), 3.73-3.56 (m, 1H), 3.29-2.87 (m, 4H), 2.59 (dq, J=13.4, 4.8, 4.3 Hz, 1H), 2.42-2.26 (m, 1H), 2.03-1.76 (m, 1H), 1.55 (d, J=13.8 Hz, 3H).

### Example 48: Synthesis of (2S,7R)—N-(1-cyano-2-{8-cyano-2-fluoro-6H-benzo[c]chromen-3-yl}ethyl)-7-methoxy-1,4-oxazocane-2-carboxamide (Compound B11)

### Step 1. Synthesis of tert-butyl (2S,7R)-2-[(1-cyano-2-{8-cyano-2-fluoro-6H-benzo[c]chromen-3-yl}ethyl)carbamoyl]-7-methoxy-1,4-oxazocane-4-carboxylate

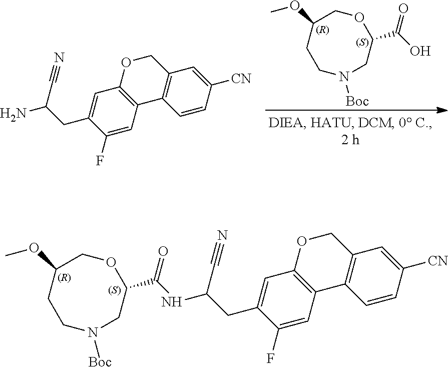

To a stirred solution of 3-(2-amino-2-cyanoethyl)-2-fluoro-6H-benzo[c]chromene-8-carbonitrile (80 mg, 0.273 mmol, 1.0 equiv) and (2S,7R)-4-(tert-butoxycarbonyl)-7-methoxy-1,4-oxazocane-2-carboxylic acid (86 mg, 0.300 mmol, 1.1 equiv) in DCM (2 mL) were added DIEA (105 mg, 0.819 mmol, 3.0 equiv) and HATU (124 mg, 0.328 mmol, 1.2 equiv) in portions at 0° C. under nitrogen atmosphere. The resulting mixture was stirred for 2 h at 0° C. under nitrogen atmosphere. The residue was purified by silica gel column chromatography, eluted with PE/THF (2:1) to afford tert-butyl (2S,7R)-2-[(1-cyano-2-{8-cyano-2-fluoro-6H-benzo[c]chromen-3-yl}ethyl)carbamoyl]-7-methoxy-1,4-oxazocane-4-carboxylate (120 mg, 77.9%) as a white solid. LCMS (ES) [M+1]⁺ m/z: 565.

### Step 2. Synthesis of (2S,7R)—N-(1-cyano-2-{8-cyano-2-fluoro-6H-benzo[c]chromen-3-yl}ethyl)-7-methoxy-1,4-oxazocane-2-carboxamide

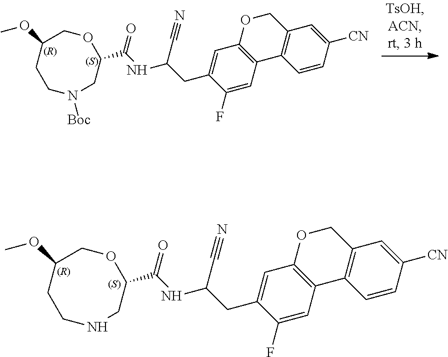

To a stirred solution of tert-butyl (2S,7R)-2-[(1-cyano-2-{8-cyano-2-fluoro-6H-benzo[c]chromen-3-yl}ethyl)carbamoyl]-7-methoxy-1,4-oxazocane-4-carboxylate (100 mg, 0.177 mmol, 1.0 equiv) in ACN (2 mL) was added TsOH (91 mg, 0.531 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound Bit (30 mg, 36.4%) as a white solid. LCMS (ES) [M+1]⁺ m/z: 465.1. ¹H NMR (300 MHz, DMSO-d6) δ 8.80-8.70 (m, 1H), 8.06 (dd, J=8.2, 2.4 Hz, 1H), 7.89 (dd, J=9.7, 3.4 Hz, 2H), 7.81 (s, 1H), 7.04 (t, J=6.8 Hz, 1H), 5.17 (d, J=5.1 Hz, 2H), 5.14-4.90 (m, 1H), 4.11 (td, J=12.1, 4.1 Hz, 1H), 3.87 (ddd, J=20.7, 9.3, 3.0 Hz, 1H), 3.65-3.45 (m, 1H), 3.31-2.89 (m, 7H), 2.32 (dd, J=14.4, 9.0 Hz, 1H), 2.04-1.82 (m, 1H), 1.56 (s, 1H).

### Example 49: Synthesis of (2S,7R)—N-{1-cyano-2-[2,8-difluoro-9-(oxetan-3-yl)-6H-benzo[c]chromen-3-yl]ethyl}-7-methoxy-1,4-oxazocane-2-carboxamide (Compound B12)

### Step 1. Synthesis of tert-butyl (2S,7R)-2-({1-cyano-2-[2,8-difluoro-9-(oxetan-3-yl)-6H-benzo[c]chromen-3-yl]ethyl}carbamoyl)-7-methoxy-1,4-oxazocane-4-carboxylate

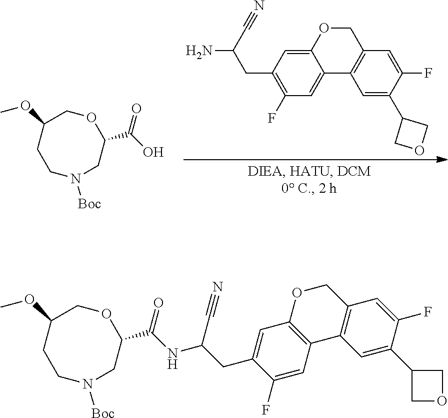 [SMILES: CO[C@@H]1CCN(C(=O)OC(C)(C)C)C[C@@H](C(=O)O)OC1 ; N#CC(N)Cc1cc2c(cc1F)-c1cc(C3COC3)c(F)cc1CO2 ; CO[C@@H]1CCN(C(=O)OC(C)(C)C)C[C@@H](C(=O)NC(C#N)Cc2cc3c(cc2F)-c2cc(C4COC4)c(F)cc2CO3)OC1]

To a stirred solution of (2S,7R)-4-(tert-butoxycarbonyl)-7-methoxy-1,4-oxazocane-2-carboxylic acid (80 mg, 0.277 mmol, 1.0 equiv) and 2-amino-3-[2,8-difluoro-9-(oxetan-3-yl)-6H-benzo[c]chromen-3-yl]propanenitrile (113 mg, 0.332 mmol, 1.2 equiv) in DCM (1.5 mL) were added DIEA (107 mg, 0.831 mmol, 3.0 equiv) and HATU (126 mg, 0.332 mmol, 1.2 equiv) in portions at 0° C. under nitrogen atmosphere. The resulting mixture was stirred at 0° C. for 2 h. The residue was purified by silica gel column chromatography, eluted with PE/THF (2:1) to afford tert-butyl (2S,7R)-2-({1-cyano-2-[2,8-difluoro-9-(oxetan-3-yl)-6H-benzo[c]chromen-3-yl]ethyl}carbamoyl)-7-methoxy-1,4-oxazocane-4-carboxylate (120 mg, 70.7%) as a white solid. LCMS (ES) [M+1]⁺ m/z: 614.

### Step 2. Synthesis of (2S,7R)—N-{1-cyano-2-[2,8-difluoro-9-(oxetan-3-yl)-6H-benzo[c]chromen-3-yl]ethyl}-7-methoxy-1,4-oxazocane-2-carboxamide

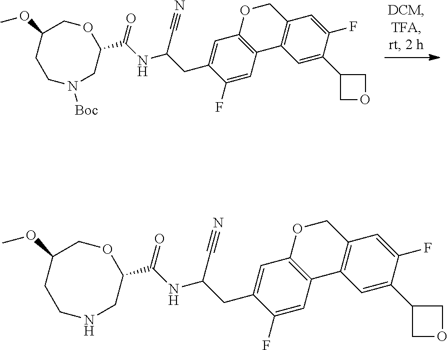 [SMILES: CO[C@@H]1CCN(C(=O)OC(C)(C)C)C[C@@H](C(=O)NC(C#N)Cc2cc3c(cc2F)-c2cc(C4COC4)c(F)cc2CO3)OC1 ; CO[C@@H]1CCNC[C@@H](C(=O)NC(C#N)Cc2cc3c(cc2F)-c2cc(C4COC4)c(F)cc2CO3)OC1]

To a stirred solution of tert-butyl (2S,7R)-2-({1-cyano-2-[2,8-difluoro-9-(oxetan-3-yl)-6H-benzo[c]chromen-3-yl]ethyl}carbamoyl)-7-methoxy-1,4-oxazocane-4-carboxylate (100 mg, 0.163 mmol, 1.0 equiv) in DCM (2 mL) was added TFA (0.2 mL). The resulting mixture was stirred at room temperature for 2 h. The mixture was basified to pH 8 with saturated NaHCO₃ (aq.). The resulting mixture was extracted with CH₂Cl₂ (3×10 mL). The combined organic layers were washed with brine (10 mL), dried over anhydrous Na₂SO₄. After filtration, the filtrate was concentrated under reduced pressure. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound B12 (30 mg, 35.8%) as a white solid. LCMS (ES) [M+1]⁺ m/z: 514. ¹H NMR (300 MHz, DMSO-d6) δ 8.72 (dd, J=8.6, 1.6 Hz, 1H), 8.02-7.89 (m, 2H), 7.18 (dd, J=10.2, 1.2 Hz, 1H), 6.98 (t, J=6.7 Hz, 1H), 5.20-4.71 (m, 7H), 4.52 (d, J=8.0 Hz, 1H), 4.13 (td, J=11.7, 4.2 Hz, 1H), 3.88 (ddd, J=21.0, 9.2, 2.9 Hz, 1H), 3.56 (ddd, J=20.5, 11.5, 8.6 Hz, 1H), 3.42-3.29 (m, 1H), 3.25 (s, 3H), 3.29-2.92 (m, 4H), 2.53-2.50 (m, 1H), 2.35 (dd, J=14.5, 9.1 Hz, 1H), 1.96-1.89 (m, 1H), 1.69-1.46 (m, 1H).

### Example 50: Synthesis of (2S,6S)—N-[(1S)-1-cyano-2-{8-cyano-2-fluoro-6H-benzo[c]chromen-3-yl}ethyl]-6-methoxy-1,4-oxazocane-2-carboxamide (Compound B13) and (2S,6S)—N-[(1R)-1-cyano-2-{8-cyano-2-fluoro-6H-benzo[c]chromen-3-yl}ethyl]-6-methoxy-1,4-oxazocane-2-carboxamide (Compound B14)

### Step 1. Synthesis of tert-butyl (2S,6S)-2-[(1-cyano-2-{8-cyano-2-fluoro-6H-benzo[c]chromen-3-yl}ethyl)carbamoyl]-6-methoxy-1,4-oxazocane-4-carboxylate

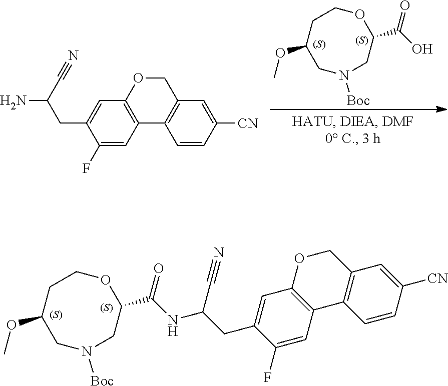

To a stirred solution of 3-(2-amino-2-cyanoethyl)-2-fluoro-6H-benzo[c]chromene-8-carbonitrile (130 mg, 0.44 mmol, 1.0 equiv) and (2S,6S)-4-(tert-butoxycarbonyl)-6-methoxy-1,4-oxazocane-2-carboxylic acid (128 mg, 0.44 mmol, 1.0 equiv), DIEA (171 mg, 1.33 mmol, 3.0 equiv) in DMF (5 mL) was added HATU (202 mg, 0.53 mmol, 1.2 equiv) in portions at 0° C. The resulting mixture was stirred for 3 h at 0° C. The reaction was quenched with water (15 mL), extracted with EtOAc (3×20 mL). The combined organic layer was washed with brine (3×30 mL), dried over anhydrous Na₂SO₄. After filtration, the filtrate was concentrated under reduced pressure. The residue was purified by silica gel column chromatography, eluted with PE/THF (1:1) to afford tert-butyl (2S,6S)-2-[(1-cyano-2-{8-cyano-2-fluoro-6H-benzo[c]chromen-3-yl}ethyl)carbamoyl]-6-methoxy-1,4-oxazocane-4-carboxylate (180 mg, 72%) as a white semi-solid. LCMS (ES, m/z): [M+H]⁺: 565.

### Step 2. Synthesis of (2S,6S)—N-(1-cyano-2-{8-cyano-2-fluoro-6H-benzo[c]chromen-3-yl}ethyl)-6-methoxy-1,4-oxazocane-2-carboxamide

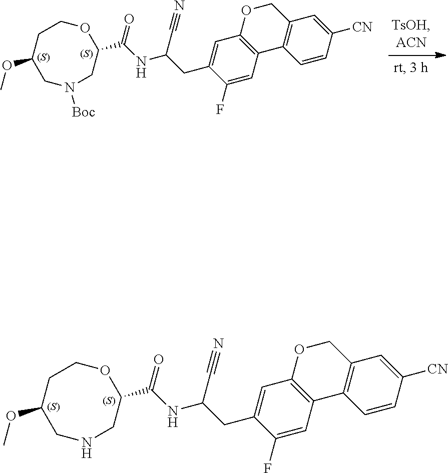

Into a 25 mL round-bottom flask were added tert-butyl (2S,6S)-2-[(1-cyano-2-{8-cyano-2-fluoro-6H-benzo[c]chromen-3-yl}ethyl)carbamoyl]-6-methoxy-1,4-oxazocane-4-carboxylate (180 mg, 0.32 mmol, 1.0 equiv), ACN (6 mL) and TsOH·H₂O (164 mg, 0.96 mmol, 3.0 equiv) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in (2S,6S)—N-(1-cyano-2-{8-cyano-2-fluoro-6H-benzo[c]chromen-3-yl}ethyl)-6-methoxy-1,4-oxazocane-2-carboxamide (120 mg, 81%) as a white solid. LCMS (ES, m/z): [M+H]⁺: 465.

### Step 3. Synthesis of (2S,6S)—N-[(1S)-1-cyano-2-{8-cyano-2-fluoro-6H-benzo[c]chromen-3-yl}ethyl]-6-methoxy-1,4-oxazocane-2-carboxamide and (2S,6S)—N-[(1R)-1-cyano-2-{8-cyano-2-fluoro-6H-benzo[c]chromen-3-yl}ethyl]-6-methoxy-1,4-oxazocane-2-carboxamide

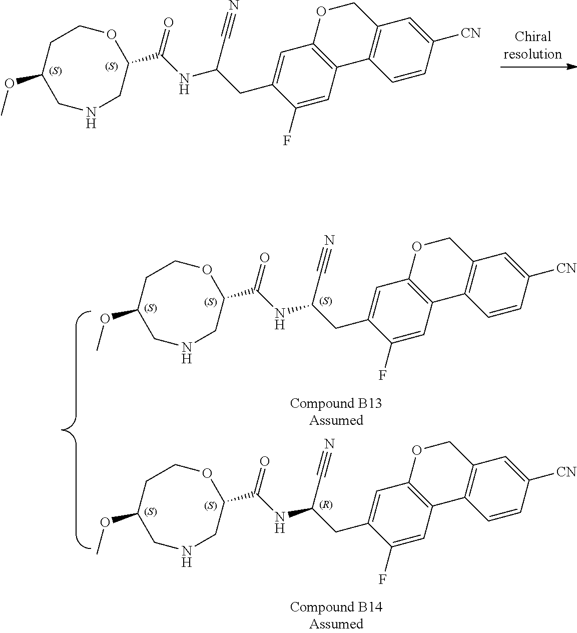

(2S,6S)—N-(1-cyano-2-{8-cyano-2-fluoro-6H-benzo[c]chromen-3-yl}ethyl)-6-methoxy-1,4-oxazocane-2-carboxamide (100 mg, 0.21 mmol, 1.0 equiv) was separated by Prep-HPLC with the following conditions Column: (R, R)-WHELK-01-Kromasil, 3*25 cm, 5 m; Mobile Phase A: CO₂, Mobile Phase B: MeOH:DCM=2:1 (0.1% 2 M NH₃-MeOH); Flow rate: 80 mL/min; Gradient: isocratic 35% B; Column Temperature (° C.): 35; BackPressure (bar): 100; Wave Length: 254 nm; RT1(min): 8.45; RT2(min): 9.85; Sample Solvent: MeOH; Injection Volume: 6 mL. This resulted in Compound B13 (25.5 mg, 25%) as a white solid and Compound B14 (18.5 mg, 18%) as a white solid. LCMS (ES, m/z): [M+H]⁺: 465.1.

Compound B13: ¹H NMR (400 MHz, DMSO-d₆) δ 8.71 (d, J=8.6 Hz, 1H), 8.06 (d, J=8.1 Hz, 1H), 7.88 (d, J=9.3 Hz, 2H), 7.80 (s, 1H), 7.01 (d, J=6.3 Hz, 1H), 5.23-5.10 (m, 2H), 5.03 (q, J=8.2 Hz, 1H), 3.91 (d, J=9.9 Hz, 1H), 3.82 (t, J=11.1 Hz, 1H), 3.69 (dt, J=12.2, 4.1 Hz, 1H), 3.28-3.09 (m, 7H), 2.98 (d, J=13.6 Hz, 1H), 2.69 (dd, J=14.9, 3.7 Hz, 1H), 2.16 (dd, J=13.8, 10.3 Hz, 2H), 1.68-1.59 (m, 1H).

Compound B14: ¹H NMR (400 MHz, DMSO-d₆) δ 8.81 (d, J=8.5 Hz, 1H), 8.06 (d, J=8.2 Hz, 1H), 7.88 (d, J=9.5 Hz, 2H), 7.81 (s, 1H), 7.04 (d, J=6.4 Hz, 1H), 5.18 (s, 2H), 4.98 (q, J=8.1 Hz, 1H), 3.96-3.89 (m, 1H), 3.89-3.79 (m, 1H), 3.70-3.61 (m, 1H), 3.40-3.30 (m, 1H), 3.28-3.11 (m, 7H), 2.84 (d, J=14.3 Hz, 1H), 2.16 (q, J=10.8 Hz, 1H), 1.72 (dd, J=18.9, 15.1 Hz, 1H).

### Example 51: Synthesis of (2S,6S)—N-{1-cyano-2-[2,8-difluoro-9-(oxetan-3-yl)-6H-benzo[c]chromen-3-yl]ethyl}-6-methoxy-1,4-oxazocane-2-carboxamide (Compound B15)

### Step 1. Synthesis of tert-butyl (2S,6S)-2-({1-cyano-2-[2,8-difluoro-9-(oxetan-3-yl)-6H-benzo[c]chromen-3-yl]ethyl}carbamoyl)-6-methoxy-1,4-oxazocane-4-carboxylate

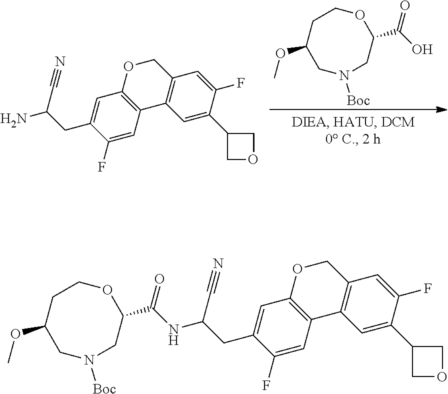

To a stirred solution of 2-amino-3-[2,8-difluoro-9-(oxetan-3-yl)-6H-benzo[c]chromen-3-yl]propanenitrile (113 mg, 0.332 mmol, 1.2 equiv) and (2S,6S)-4-(tert-butoxycarbonyl)-6-methoxy-1,4-oxazocane-2-carboxylic acid (80 mg, 0.277 mmol, 1.0 equiv) in DCM (2 mL) were added DIEA (107 mg, 0.831 mmol, 3.0 equiv) and HATU (126 mg, 0.332 mmol, 1.2 equiv) in portions at 0° C. under nitrogen atmosphere. The resulting mixture was stirred at 0° C. for 2 h. The residue was purified by silica gel column chromatography, eluted with PE/THF (2:1) to afford tert-butyl (2S,6S)-2-({1-cyano-2-[2,8-difluoro-9-(oxetan-3-yl)-6H-benzo[c]chromen-3-yl]ethyl}carbamoyl)-6-methoxy-1,4-oxazocane-4-carboxylate (120 mg, 70.7%) as a white solid. LCMS (ES) [M+1]⁺ m/z: 614.

### Step 2. Synthesis of (2S,6S)—N-{1-cyano-2-[2,8-difluoro-9-(oxetan-3-yl)-6H-benzo[c]chromen-3-yl-]ethyl}-6-methoxy-1,4-oxazocane-2-carboxamide

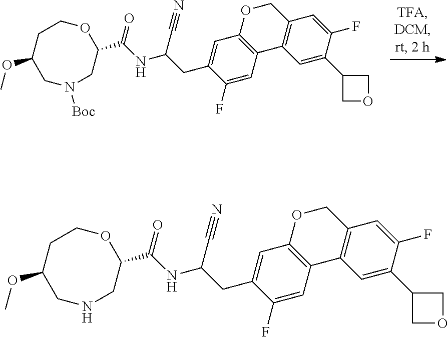 [SMILES: CO[C@H]1CCO[C@H](C(=O)NC(C#N)Cc2cc3c(cc2F)-c2cc(C4COC4)c(F)cc2CO3)CN(C(=O)OC(C)(C)C)C1 ; CO[C@H]1CCO[C@H](C(=O)NC(C#N)Cc2cc3c(cc2F)-c2cc(C4COC4)c(F)cc2CO3)CNC1]

To a stirred solution of tert-butyl (2S,6S)-2-({1-cyano-2-[2,8-difluoro-9-(oxetan-3-yl)-6H-benzo[c]chromen-3-yl]ethyl}carbamoyl)-6-methoxy-1,4-oxazocane-4-carboxylate (100 mg, 0.163 mmol, 1.0 equiv) in DCM (2 mL) was added TFA (0.2 mL). The resulting mixture was stirred at room temperature for 2 h. The mixture was basified to pH 8 with saturated NaHCO₃ (aq.). The resulting mixture was extracted with CH₂Cl₂ (3×5 mL). The combined organic layers were washed with brine (5 mL), dried over anhydrous Na₂SO₄. After filtration, the filtrate was concentrated under reduced pressure. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound B15 (30 mg, 35.8%) as a white solid. LCMS (ES) [M+1]⁺ m/z: 514. ¹H NMR (300 MHz, DMSO-d6) δ 8.70 (dd, J=8.6, 2.7 Hz, 1H), 8.02-7.88 (m, 2H), 7.18 (d, J=10.2 Hz, 1H), 6.96 (t, J=7.0 Hz, 1H), 5.09 (d, J=3.3 Hz, 2H), 5.03-4.80 (m, 5H), 4.53 (p, J=8.0 Hz, 1H), 4.09-3.73 (m, 2H), 3.74-3.59 (m, 1H), 3.32 (s, 3H), 3.28-2.87 (m, 5H), 2.69 (dt, J=14.7, 3.9 Hz, 1H), 2.41-2.07 (m, 3H), 1.69-1.55 (m, 1H).

### Example 52: Synthesis of (2S)—N-(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}ethyl)-1,4-oxazepane-2-carboxamide (Compound C1)

### Step 1. Synthesis of 2-amino-4-bromo-5-methylphenol

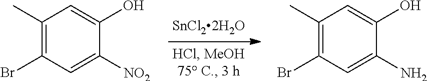

To a solution of 4-bromo-5-methyl-2-nitrophenol (10 g, 43.09 mmol, 1.0 equiv) in MeOH (100 mL) was added HCl (c) (9.2 mL). This was followed by the addition of SnCl₂·2H₂O (39.3 g, 172.38 mmol, 4.0 equiv). The mixture was stirred for 3 h at 75° C. The reaction was cooled to room temperature, the resulting mixture was concentrated under reduced pressure. The mixture was basified to pH 14 with NH₃·H₂O extracted with EtOAc (3×300 mL). The combined organic layer was washed with brine (3×300 mL), dried over anhydrous Na₂SO₄. After filtration, the filtrate was concentrated under reduced pressure. This resulted in 2-amino-4-bromo-5-methylphenol (8.0 g, 91.8%) as a yellow solid. LCMS (ES, m/z): [M+H]⁺: 202. ¹H NMR (400 MHz, DMSO-d₆) δ 9.15 (brs, 1H), 6.76 (s, 1H), 6.58 (s, 1H), 4.57 (brs, 2H), 2.11 (s, 3H).

### Step 2. Synthesis of 5-bromo-6-methyl-3H-1,3-benzoxazol-2-one

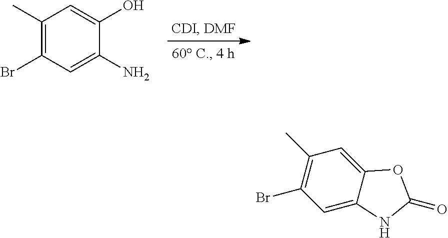

To a stirred solution of 2-amino-4-bromo-5-methylphenol (8.0 g, 39.59 mmol, 1.0 equiv) in DMF (80 mL) was added CDI (7.7 g, 47.51 mmol, 1.2 equiv) in DMF (50 mL) dropwise at 0° C. under nitrogen atmosphere. The resulting mixture was stirred for additional 3 h at 60° C. The reaction was cooled to room temperature, diluted with water (150 mL), extracted with EtOAc (3×300 mL). The combined organic layer was washed with brine (3×500 mL), dried over anhydrous Na₂SO₄. After filtration, the filtrate was concentrated under reduced pressure. This resulted in 5-bromo-6-methyl-3H-1,3-benzoxazol-2-one (8.0 g, 88.6%) as a yellow solid. LCMS (ES, m/z): [M+H]⁺: 228. ¹H NMR (400 MHz, DMSO-d₆) δ 7.33 (s, 1H), 7.26 (s, 1H), 2.33 (s, 3H).

### Step 3. Synthesis of 5-bromo-3,6-dimethyl-1,3-benzoxazol-2-one

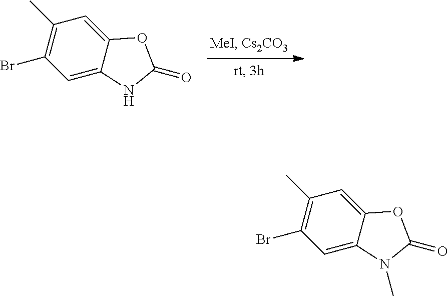

To a stirred mixture of 5-bromo-6-methyl-3H-1,3-benzoxazol-2-one (8.0 g, 35.08 mmol, 1.0 equiv) and Cs₂CO₃ (22.8 g, 70.16 mmol, 2.0 equiv) in DMF (80 mL) was added MeI (9.9 g, 70.16 mmol, 2.0 equiv) dropwise at 0° C. The resulting mixture was stirred for 3 h at room temperature. The resulting mixture was filtered, the filter cake was washed with EtOAc (3×100 mL). The filtrate was diluted with water (100 mL), extracted with EtOAc (2×300 mL). The combined organic layer was washed with brine (3×300 mL), dried over anhydrous Na₂SO₄. After filtration, the filtrate was concentrated under reduced pressure. The residue was purified by trituration with PE/EA=10:1 (50 mL). This resulted in 5-bromo-3,6-dimethyl-1,3-benzoxazol-2-one (6.6 g, 77.7%) as a yellow solid. LCMS (ES, m/z): [M+H]⁺: 242. ¹H NMR (300 MHz, DMSO-d₆) δ 7.57 (s, 1H), 7.39 (s, 1H), 3.32 (s, 3H), 2.36 (s, 3H).

### Step 4. Synthesis of 5-bromo-6-(bromomethyl)-3-methyl-1,3-benzoxazol-2-one

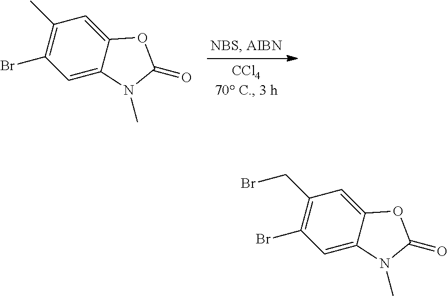

A solution of 5-bromo-3,6-dimethyl-1,3-benzoxazol-2-one (6.6 g, 27.26 mmol, 1.0 equiv) and NBS (5.8 g, 32.71 mmol, 1.2 equiv), AIBN (0.5 g, 2.72 mmol, 0.1 equiv) in CCl₄ (70 mL) was stirred for 3 h at 70° C. The resulting mixture was concentrated under reduced pressure. The residue was purified by silica gel column chromatography, eluted with PE/EA (10:1) to afford 5-bromo-6-(bromomethyl)-3-methyl-1,3-benzoxazol-2-one (6.2 g, 70.8%) as a yellow solid. LCMS (ES, m/z): [M+H]⁺: 320. ¹H NMR (400 MHz, DMSO-d₆) δ 7.66 (s, 1H), 7.65 (s, 1H), 4.79 (s, 2H), 3.33 (s, 3H).

### Step 5. Synthesis of methyl 5-[(5-bromo-3-methyl-2-oxo-1,3-benzoxazol-6-yl)methoxy]-2-fluorobenzoate

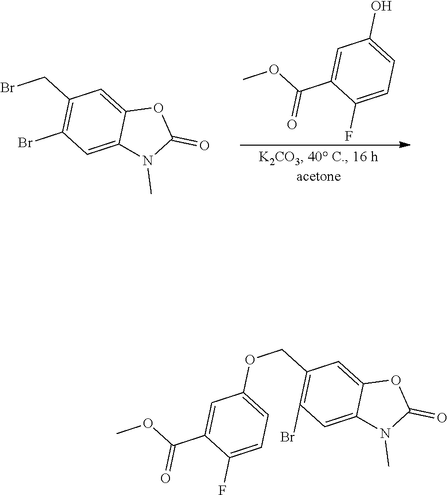 [SMILES: Cn1c(=O)oc2cc(CBr)c(Br)cc21 ; COC(=O)c1cc(O)ccc1F ; COC(=O)c1cc(OCc2cc3oc(=O)n(C)c3cc2Br)ccc1F]

A mixture of 5-bromo-6-(bromomethyl)-3-methyl-1,3-benzoxazol-2-one (6.2 g, 19.31 mmol, 1.0 equiv), methyl 2-fluoro-5-hydroxybenzoate (3.0 g, 19.31 mmol, 1.0 equiv) and K₂CO₃ (5.3 g, 38.62 mmol, 2.0 equiv) in acetone (70 mL) was stirred for 16 h at 40° C. The resulting mixture was diluted with H₂O (50 mL). The precipitated solid were collected by filtration and washed with H₂O (3×20 mL), dried under infrared lamp for 4 h. This resulted in methyl 5-[(5-bromo-3-methyl-2-oxo-1,3-benzoxazol-6-yl)methoxy]-2-fluorobenzoate (9 g, crude) as a white solid. LCMS (ES, m/z): [M+H]⁺: 410. ¹H NMR (400 MHz, DMSO-d₆) δ 7.69 (s, 1H), 7.62 (s, 1H), 7.47-7.44 (m, 1H), 7.38-7.29 (m, 2H), 5.16 (s, 2H), 3.86 (s, 3H), 3.35 (s, 3H).

### Step 6. Synthesis of methyl 4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaene-5-carboxylate

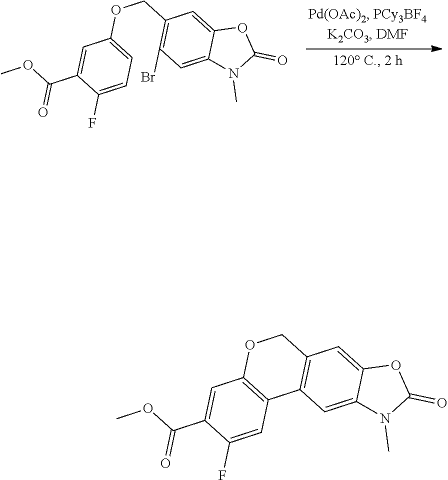 [SMILES: COC(=O)c1cc(OCc2cc3oc(=O)n(C)c3cc2Br)ccc1F ; COC(=O)c1cc2c(cc1F)-c1cc3c(cc1CO2)oc(=O)n3C]

A mixture of methyl 5-[(5-bromo-3-methyl-2-oxo-1,3-benzoxazol-6-yl)methoxy]-2-fluorobenzoate (9.0 g, 21.94 mmol, 1.0 equiv) and Pd(OAc)₂ (0.5 g, 2.19 mmol, 0.1 equiv), K₂CO₃ (6.1 g, 43.88 mmol, 2.0 equiv), PCy₃BF₄ (1.6 g, 4.38 mmol, 0.2 equiv) in DMF (90 mL) was stirred for 2 h at 120° C. under nitrogen atmosphere. The reaction was cooled to room temperature, diluted with water (100 mL), extracted with EtOAc (3×300 mL). The combined organic layer was washed with brine (3×500 mL), dried over anhydrous Na₂SO₄. After filtration, the filtrate was concentrated under reduced pressure. This resulted in methyl 4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaene-5-carboxylate (4 g, 55.3%) as a dark grey solid. LCMS (ES, m/z): [M+H]⁺: 330. ¹H NMR (400 MHz, DMSO-d₆) δ 7.96 (d, J=11.7 Hz, 1H), 7.93 (s, 1H), 7.39 (d, J=6.2 Hz, 1H), 7.35 (s, 1H), 5.19 (s, 2H), 3.86 (s, 3H), 3.40 (s, 3H).

### Step 7. Synthesis of 4-fluoro-5-(hydroxymethyl)-15-methyl-8,13-dioxa-15-azatetracyclo[8.7.0.0\hat{ }{2,7}0.0\hat{ }{12,16}]heptadeca-1(10),2(7),3,5,11,16-hexaen-14-one

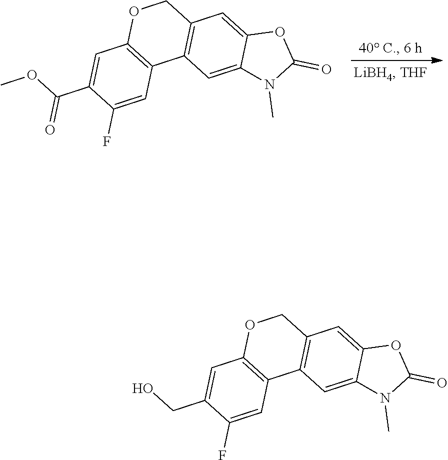 [SMILES: COC(=O)c1cc2c(cc1F)-c1cc3c(cc1CO2)oc(=O)n3C ; Cn1c(=O)oc2cc3c(cc21)-c1cc(F)c(CO)cc1OC3]

To a stirred solution of methyl 4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaene-5-carboxylate (3.5 g, 10.62 mmol, 1.0 equiv) in THF (40 mL) was added LiBH₄ (0.7 g, 31.88 mmol, 3.0 equiv) at 0° C. The resulting mixture was stirred for additional 6 h at room temperature. The residue was purified by trituration with DMF/H₂O=1:3 (5.6 mL). This resulted in 4-fluoro-5-(hydroxymethyl)-15-methyl-8,13-dioxa-15-azatetracyclo[8.7.0.0\hat{ }{2,7}0.0\hat{ }{12,16}]heptadeca-1(10),2(7),3,5,11,16-hexaen-14-one (900 mg, 28.1%) as a dark grey solid. ¹H NMR (400 MHz, DMSO-d₆) δ 7.82 (s, 1H), 7.77 (d, J=10.9 Hz, 1H), 7.31 (s, 1H), 7.04 (d, J=6.4 Hz, 1H), 5.32 (brs, 1H), 5.11 (s, 2H), 4.54 (d, J=3.9 Hz, 2H), 3.40 (s, 3H).

### Step 8. Synthesis of 5-(bromomethyl)-4-fluoro-15-methyl-8,13-dioxa-15-azatetracyclo[8.7.0.0\hat{ }{2,7}0.0\hat{ }{12,16}]heptadeca-1(10),2(7),3,5,11,16-hexaen-14-one

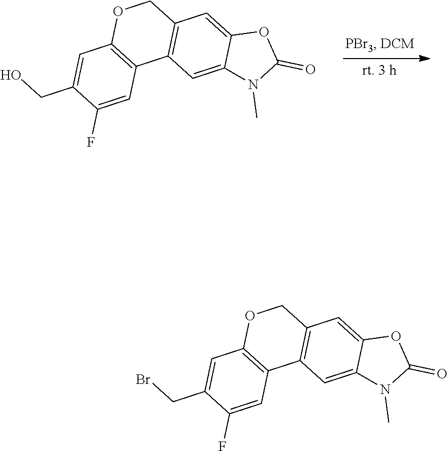 [SMILES: Cn1c(=O)oc2cc3c(cc21)-c1cc(F)c(CO)cc1OC3 ; Cn1c(=O)oc2cc3c(cc21)-c1cc(F)c(CBr)cc1OC3]

To a stirred solution of 4-fluoro-5-(hydroxymethyl)-15-methyl-8,13-dioxa-15-azatetracyclo[8.7.0.0\hat{ }{2,7}0.0\hat{ }{12,16}]heptadeca-1(10),2(7),3,5,11,16-hexaen-14-one (900 mg, 2.98 mmol, 1.0 equiv) in DCM (9 mL) was added PBr₃ (404 mg, 1.49 mmol, 0.5 equiv) dropwise at 0° C. The resulting mixture was stirred for 3 h at room temperature. The reaction was quenched with water (20 mL), extracted with CH₂Cl₂ (3×30 mL). The combined organic layer was washed with brine (3×30 mL), dried over anhydrous Na₂SO₄. After filtration, the filtrate was concentrated under reduced pressure, to afford 5-(bromomethyl)-4-fluoro-15-methyl-8,13-dioxa-15-azatetracyclo[8.7.0.0\hat{ }{2,7}0.0\hat{ }{12,16}]heptadeca-1(10),2(7),3,5,11,16-hexaen-14-one (800 mg, 73.5%) as a yellow solid and used to the next step directly without further purification. ¹H NMR (300 MHz, DMSO-d₆) δ 7.89-7.81 (m, 2H), 7.31 (s, 1H), 7.16 (d, J=6.7 Hz, 1H), 5.12 (s, 2H), 4.67 (s, 2H), 3.38 (s, 3H).

### Step 9. Synthesis of 2-[(diphenylmethylidene)amino]-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile

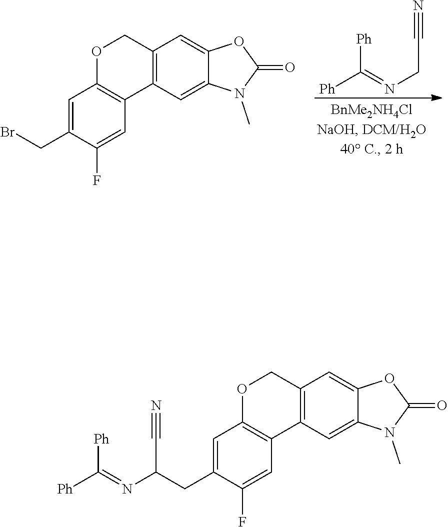

A solution of 5-(bromomethyl)-4-fluoro-15-methyl-8,13-dioxa-15-azatetracyclo[8.7.0.0\hat{ }{2,7}0.0\hat{ }{12,16}]heptadeca-1(10),2(7),3,5,11,16-hexaen-14-one (800 mg, 2.19 mmol, 1.0 equiv) in DCM (8 mL) was added 2-[(diphenylmethylidene)amino]acetonitrile (387 mg, 1.75 mmol, 0.8 equiv), benzyltrimethylazanium chloride (41 mg, 0.22 mmol, 0.1 equiv), NaOH (176 mg, 4.39 mmol, 2.0 equiv) in H₂O (0.8 mL) was stirred for 2 h at 40° C. The resulting mixture was extracted with CH₂Cl₂ (3×20 mL). The combined organic layer was washed with brine (3×20 mL), dried over anhydrous Na₂SO₄. After filtration, the filtrate was concentrated under reduced pressure. The residue was purified by silica gel column chromatography, eluted with PE/THF (5:1) to afford 2-[(diphenylmethylidene)amino]-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile (80 mg, 7%) as a yellow solid. LCMS (ES, m/z): [M+H]⁺: 504.

### Step 10. Synthesis of 2-amino-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile

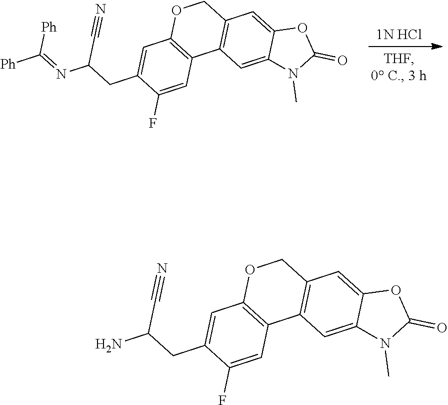

Into a 8 mL vial were added 2-[(diphenylmethylidene)amino]-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile (80 mg, 0.16 mmol, 1.0 equiv) and HCl (1 M) (0.18 mL), THF (0.4 mL)/H₂O (0.04 mL) at room temperature. The resulting mixture was stirred for 3 h at room temperature. The mixture was basified to pH 12 with K₂CO₃(aq). The resulting mixture was extracted with EtOAc (3×10 mL). The combined organic layer was washed with brine (3×10 mL), dried over anhydrous Na₂SO₄. After filtration, the filtrate was concentrated under reduced pressure. The residue was purified by silica gel column chromatography, eluted with PE/THF (1:1) to afford 2-amino-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile (35 mg, 65%) as a yellow solid. LCMS (ES, m/z): [M+H]⁺: 340.

### Step 11. Synthesis of tert-butyl (2S)-2-[(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}ethyl)carbamoyl]-1,4-oxazepane-4-carboxylate

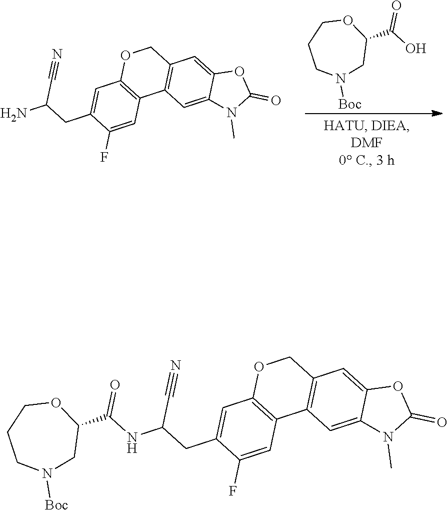 [SMILES: Cn1c(=O)oc2cc3c(cc21)-c1cc(F)c(CC(N)C#N)cc1OC3 ; CC(C)(C)OC(=O)N1CCCO[C@H](C(=O)O)C1 ; Cn1c(=O)oc2cc3c(cc21)-c1cc(F)c(CC(C#N)NC(=O)[C@@H]2CN(C(=O)OC(C)(C)C)CCCO2)cc1OC3]

To a stirred solution of (2S)-4-(tert-butoxycarbonyl)-1,4-oxazepane-2-carboxylic acid (19 mg, 0.07 mmol, 1.0 equiv) and 2-amino-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile (34 mg, 0.10 mmol, 1.3 equiv), DIEA (30 mg, 0.23 mmol, 3.0 equiv) in DMF (2 mL) were added HATU (35 mg, 0.09 mmol, 1.2 equiv) in portions at 0° C. The resulting mixture was stirred for 3 h at 0° C. The resulting mixture was extracted with EtOAc (3×10 mL). The combined organic layer was washed with brine (3×10 mL), dried over anhydrous Na₂SO₄. After filtration, the filtrate was concentrated under reduced pressure. The residue was purified by silica gel column chromatography, eluted with PE/THF (1:1) to afford tert-butyl (2S)-2-[(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}ethyl)carbamoyl]-1,4-oxazepane-4-carboxylate (20 mg, 45.6%) as a light yellow solid. LCMS (ES, m/z): [M+H]⁺: 567.

### Step 12. Synthesis of (2S)—N-(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}ethyl)-1,4-oxazepane-2-carboxamide

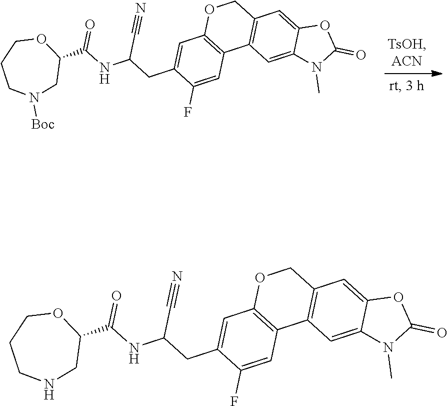 [SMILES: Cn1c(=O)oc2cc3c(cc21)-c1cc(F)c(CC(C#N)NC(=O)[C@@H]2CN(C(=O)OC(C)(C)C)CCCO2)cc1OC3 ; Cn1c(=O)oc2cc3c(cc21)-c1cc(F)c(CC(C#N)NC(=O)[C@@H]2CNCCCO2)cc1OC3]

Into a 8 mL vial were added tert-butyl (2S)-2-[(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}ethyl)carbamoyl]-1,4-oxazepane-4-carboxylate (20 mg, 0.03 mmol, 1.0 equiv) in ACN (1 mL) and TsOH·H₂O (20 mg, 0.10 mmol, 3.0 equiv) at room temperature. The resulting mixture was stirred for 3 h at room temperature. The residue was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound C1 (4.8 mg, 29%) as a white solid. LCMS (ES, m/z): [M+H]⁺: 467.1. ¹H NMR (400 MHz, DMSO-d₆) δ 8.68 (dd, J=12.0, 8.5 Hz, 1H), 7.86-7.78 (m, 2H), 7.32 (s, 1H), 6.99 (d, J=6.1 Hz, 1H), 5.12 (d, J=4.0 Hz, 2H), 5.08-4.94 (m, 1H), 4.04-3.81 (m, 2H), 3.77-3.71 (m, 1H), 3.43 (s, 3H), 3.25-3.00 (m, 3H), 2.85-2.56 (m, 4H), 1.92-1.66 (m, 2H).

### Example 53: Synthesis of (2S)—N-(1-cyano-2-(8-fluoro-3-methyl-2-oxo-3,4-dihydro-2H-benzo[3,4]isochromeno[8,7-d]oxazol-7-yl)ethyl)-1,4-oxazepane-2-carboxamide (Compound C2)

### Step 1. Synthesis of 2-amino-4-bromo-3-methylphenol

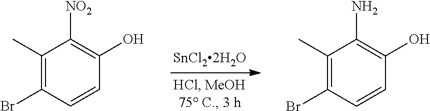

To a solution of 4-bromo-3-methyl-2-nitrophenol (8.7 g, 37.49 mmol, 1.0 equiv) and SnCl₂·2H₂O (34.1 g, 149.98 mmol, 4.0 equiv) in MeOH (90 mL) was added HCl (c) (8.7 mL). The reaction was stirred for 3 h at 75° C. The resulting mixture was concentrated under reduced pressure. The residue was basified to pH 14 with NH₃·H₂O. The resulting mixture was extracted with EtOAc (3×300 mL). The combined organic layer was washed with brine (3×300 mL), dried over anhydrous Na₂SO₄. After filtration, the filtrate was concentrated under reduced pressure. This resulted in 2-amino-4-bromo-3-methylphenol (6.7 g, 88.5%) as a yellow solid. LCMS (ES, m/z): [M+H]⁺: 202. ¹H NMR (300 MHz, DMSO-d₆) δ 9.35 (brs, 1H), 6.65 (d, J=8.4 Hz, 1H), 6.49 (d, J=8.4 Hz, 1H), 4.57 (br, 2H), 2.15 (s, 3H).

### Step 2. Synthesis of 5-bromo-4-methyl-3H-1,3-benzoxazol-2-one

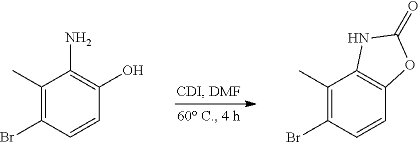

To a stirred solution of 2-amino-4-bromo-3-methylphenol (6.7 g, 33.16 mmol, 1.0 equiv) in DMF (120 mL) was added CDI (6.45 g, 39.79 mmol, 1.2 equiv) in DMF (120 mL) dropwise at 0° C. The resulting mixture was stirred for additional 4 h at 60° C. The reaction was cooled to room temperature, diluted with water (300 mL), extracted with EtOAc (3×300 mL). The combined organic layer was washed with brine (3×500 mL), dried over anhydrous Na₂SO₄. After filtration, the filtrate was concentrated under reduced pressure. This resulted in 5-bromo-4-methyl-3H-1,3-benzoxazol-2-one (6.7 g, 88.6%) as a yellow solid. LCMS (ES, m/z): [M+H]⁺: 228. ¹H NMR (300 MHz, DMSO-d₆) δ 11.94 (br, 1H), 7.30 (d, J=8.5 Hz, 1H), 7.09 (d, J=8.5 Hz, 1H), 2.32 (s, 3H).

### Step 3. Synthesis of 5-bromo-3,4-dimethyl-1,3-benzoxazol-2-one

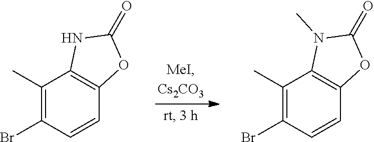

To a stirred mixture of 5-bromo-4-methyl-3H-1,3-benzoxazol-2-one (6.7 g, 29.38 mmol, 1.0 equiv) and Cs₂CO₃ (19.2 g, 58.76 mmol, 2.0 equiv) in DMF (70 mL) was added MeI (8.34 g, 58.76 mmol, 2.0 equiv) dropwise at 0° C. The resulting mixture was stirred for additional 3 h at room temperature. The resulting mixture was filtered, the filter cake was washed with EtOAc (3×100 mL). The filtrate was concentrated under reduced pressure. The resulting mixture was extracted with EtOAc (3×300 mL). The combined organic layer was washed with brine (3×500 mL), dried over anhydrous Na₂SO₄. After filtration, the filtrate was concentrated under reduced pressure. The residue was purified by trituration with PE/EA=10:1 (50 mL). This resulted in 5-bromo-3,4-dimethyl-1,3-benzoxazol-2-one (6 g, 84.4%) as a yellow solid. LCMS (ES, m/z): [M+H]⁺: 242. ¹H NMR (300 MHz, DMSO-d₆) δ 7.38 (d, J=8.5 Hz, 1H), 7.16 (d, J=8.5 Hz, 1H), 3.56 (s, 3H), 2.59 (s, 3H).

### Step 4. Synthesis of 5-bromo-4-(bromomethyl)-3-methyl-1,3-benzoxazol-2-one

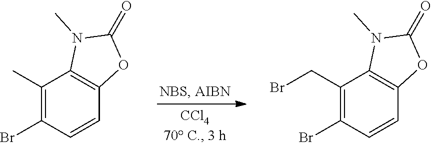

A solution of 5-bromo-3,4-dimethyl-1,3-benzoxazol-2-one (6 g, 24.78 mmol, 1.0 equiv) and NBS (5.29 g, 29.74 mmol, 1.2 equiv), AIBN (0.41 g, 2.47 mmol, 0.1 equiv) in CCl₄ (60 mL) was stirred for 3 h at 70° C. The resulting mixture was concentrated under reduced pressure. The residue was purified by silica gel column chromatography, eluted with PE/EA (10:1) to afford 5-bromo-4-(bromomethyl)-3-methyl-1,3-benzoxazol-2-one (7 g, 88%) as a yellow solid. LCMS (ES, m/z): [M+H]⁺: 320. ¹H NMR (300 MHz, DMSO-d₆) δ 7.46 (d, J=8.5 Hz, 1H), 7.32 (d, J=8.5 Hz, 1H), 4.98 (s, 2H), 3.69 (s, 3H).

### Step 5. Synthesis of methyl 5-[(5-bromo-3-methyl-2-oxo-1,3-benzoxazol-4-yl)methoxy]-2-fluorobenzoate

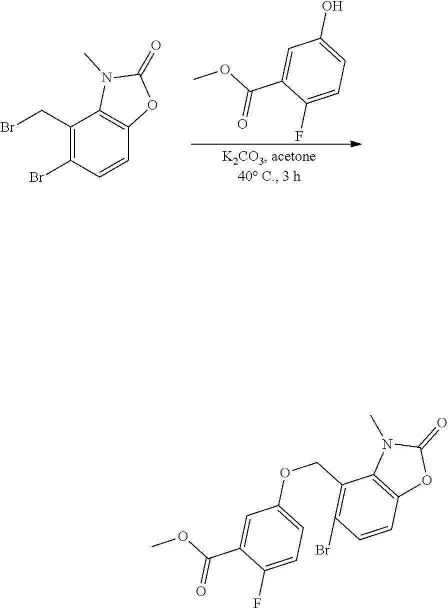 [SMILES: Cn1c(=O)oc2ccc(Br)c(CBr)c21 ; COC(=O)c1cc(O)ccc1F ; COC(=O)c1cc(OCc2c(Br)ccc3oc(=O)n(C)c23)ccc1F]

A mixture of 5-bromo-4-(bromomethyl)-3-methyl-1,3-benzoxazol-2-one (7 g, 21.81 mmol, 1.0 equiv) and methyl 2-fluoro-5-hydroxybenzoate (3.71 g, 21.80 mmol, 1.0 equiv) and K₂CO₃ (6.03 g, 43.61 mmol, 2.0 equiv) in acetone (70 mL) was stirred for 3 h at 40° C. The resulting mixture was diluted with H₂O (50 mL). The precipitated solid was collected by filtration and washed with H₂O (3×20 mL). This resulted in methyl 5-[(5-bromo-3-methyl-2-oxo-1,3-benzoxazol-4-yl)methoxy]-2-fluorobenzoate (8.1 g, 90.5%) as a white solid. LCMS (ES, m/z): [M+H]⁺: 410.

### Step 6. Synthesis of 3-{2-cyano-2-[(diphenylmethylidene)amino]ethyl}-4-fluoro-6H-benzo[c]chromene-8-carbonitrile

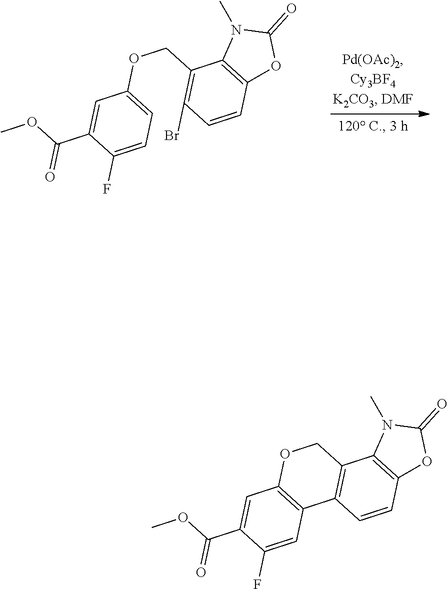 [SMILES: COC(=O)c1cc(OCc2c(Br)ccc3oc(=O)n(C)c23)ccc1F ; COC(=O)c1cc2c(cc1F)-c1ccc3oc(=O)n(C)c3c1CO2]

To a mixture of methyl 5-[(5-bromo-3-methyl-2-oxo-1,3-benzoxazol-4-yl)methoxy]-2-fluorobenzoate (8.1 g, 19.75 mmol, 1.0 equiv), PCy₃HBF₄ (0.73 g, 1.98 mmol, 0.1 equiv), K₂CO₃ (5.46 g, 39.50 mmol, 2.0 equiv) in DMF (80 mL) was added and Pd(OAc)₂ (0.44 g, 1.98 mmol, 0.1 equiv) under Nitrogen gas. The reaction was stirred for 3 h at 120° C. under nitrogen atmosphere. The reaction was cooled to room temperature, diluted with water (100 mL). The resulting mixture was extracted with EtOAc (3×300 mL). The combined organic layer was washed with brine (3×500 mL), dried over anhydrous Na₂SO₄. After filtration, the filtrate was concentrated under reduced pressure. This resulted in 3-{2-cyano-2-[(diphenylmethylidene)amino]ethyl}-4-fluoro-6H-benzo[c]chromene-8-carbonitrile (4.7 g, 72.3%) as a dark grey solid. LCMS (ES, m/z): [M+H]⁺: 330.

### Step 7. Synthesis of 5-(bromomethyl)-4-fluoro-12-methyl-8,14-dioxa-12-azatetracyclo[8.7.0.0\hat{ }{2,7}0.0\hat{ }{11,15}]heptadeca-1(10),2(7),3,5,11(15),16-hexaen-13-one

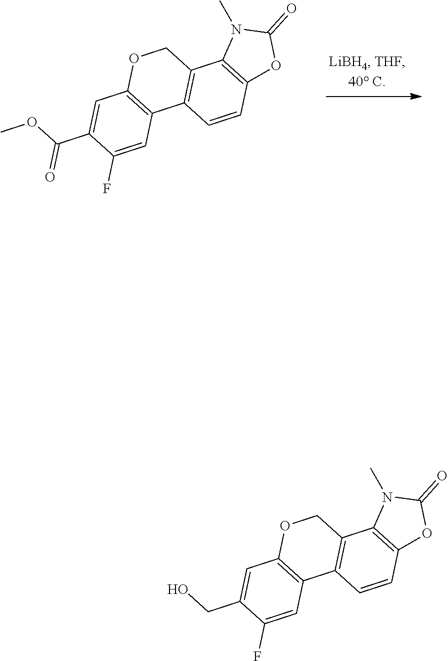 [SMILES: COC(=O)c1cc2c(cc1F)-c1ccc3oc(=O)n(C)c3c1CO2 ; Cn1c(=O)oc2ccc3c(c21)COc1cc(CO)c(F)cc1-3]

To a stirred solution of 3-{2-cyano-2-[(diphenylmethylidene)amino]ethyl}-4-fluoro-6H-benzo[c]chromene-8-carbonitrile (3.5 g, 10.62 mmol, 1.0 equiv) in THF (40 mL) was added LiBH₄ (0.20 g, 9.11 mmol, 3.0 equiv) dropwise at 0° C. The resulting mixture was stirred for additional 6 h at 40° C. The reaction was cooled to 0° C., quenched with water (50 mL). The resulting mixture was extracted with EtOAc (3×100 mL). The combined organic layer was washed with brine (3×100 mL), dried over anhydrous Na₂SO₄. After filtration, the filtrate was concentrated under reduced pressure. This resulted in 4-fluoro-5-(hydroxymethyl)-12-methyl-8,14-dioxa-12-azatetracyclo[8.7.0.0\hat{ }{2,7}0.0\hat{ }{11,15}]heptadeca-1(10),2(7),3,5,11(15),16-hexaen-13-one (700 mg, 76.51%) as a light yellow solid. LCMS (ES, m/z): [M+H+CH₃CN]⁺: 343. ¹H NMR (300 MHz, DMSO-d₆) δ 7.69 (d, J=10.9 Hz, 1H), 7.64 (d, J=8.4 Hz, 1H), 7.38 (d, J=8.3 Hz, 1H), 7.07 (d, J=6.5 Hz, 1H), 5.47 (s, 2H), 5.32 (t, J=5.8 Hz, 1H), 4.54 (d, J=5.7 Hz, 2H), 3.54 (s, 3H).

### Step 8. Synthesis of 5-(bromomethyl)-4-fluoro-12-methyl-8,14-dioxa-12-azatetracyclo[8.7.0.0\hat{ }{2,7}0.0\hat{ }{11,15}]heptadeca-1(10),2(7),3,5,11(15),16-hexaen-13-one

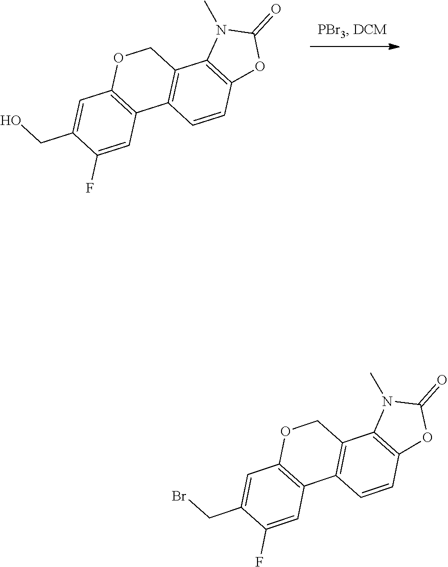 [SMILES: Cn1c(=O)oc2ccc3c(c21)COc1cc(CO)c(F)cc1-3 ; Cn1c(=O)oc2ccc3c(c21)COc1cc(CBr)c(F)cc1-3]

To a stirred solution of 4-fluoro-5-(hydroxymethyl)-12-methyl-8,14-dioxa-12-azatetracyclo[8.7.0.0\hat{ }{2,7}0.0\hat{ }{11,15}]heptadeca-1(10),2(7),3,5,11(15),16-hexaen-13-one (700 mg, 2.32 mmol, 1.0 equiv) in DCM (10 mL) was added PBr₃ (314 mg, 1.16 mmol, 0.5 equiv) dropwise at 0° C. The resulting mixture was stirred for additional 3 h at room temperature. The reaction was quenched with water (20 mL), The resulting mixture was extracted with EtOAc (3×20 mL). The combined organic layer was washed with brine (3×30 mL), dried over anhydrous Na₂SO₄. After filtration, the filtrate was concentrated under reduced pressure to afford 5-(bromomethyl)-4-fluoro-12-methyl-8,14-dioxa-12-azatetracyclo[8.7.0.0\hat{ }{2,7}0.0\hat{ }{11,15}]heptadeca-1(10),2(7),3,5,11(15),16-hexaen-13-one (0.8 g, 94.6%) as a yellow solid which was used to the next step without further purification (no MS signal).

### Step 9. Synthesis of 2-[(diphenylmethylidene)amino]-3-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}propanenitrile

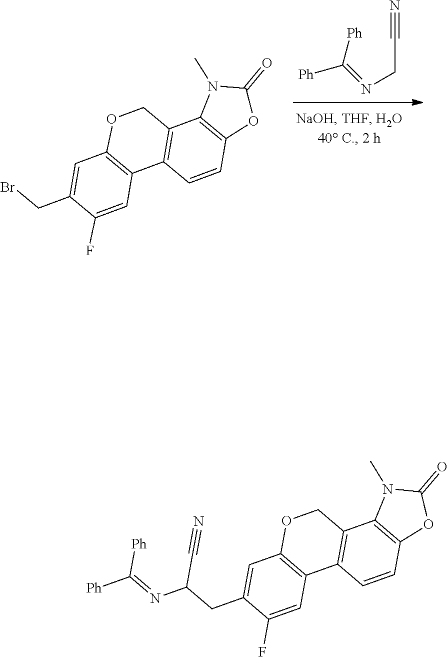

A solution of 5-(bromomethyl)-4-fluoro-12-methyl-8,14-dioxa-12-azatetracyclo[8.7.0.0\hat{ }{2,7}0.0\hat{ }{11,15}]heptadeca-1(10),2(7),3,5,11(15),16-hexaen-13-one (800 mg, 2.19 mmol, 1.0 equiv) in THF (15 mL) was added 2-[(diphenylmethylidene)amino]acetonitrile (483 mg, 2.19 mmol, 1.0 equiv), benzyltrimethylazanium chloride (41 mg, 0.22 mmol, 0.1 equiv), NaOH (175 mg, 4.39 mmol, 2.0 equiv) in H₂O (1.5 mL) was stirred for 2 h at 40° C. The resulting mixture was extracted with EtOAc (3×50 mL). The combined organic layer was washed with brine (3×100 mL), dried over anhydrous Na₂SO₄. After filtration, the filtrate was concentrated under reduced pressure. The residue was purified by silica gel column chromatography, eluted with PE/THF (5:1) to afford 2-[(diphenylmethylidene)amino]-3-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}propanenitrile (300 mg, 27%) as a light yellow solid. LCMS (ES, m/z): [M+H]⁺: 504.

### Step 10. Synthesis of 2-amino-3-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}propanenitrile

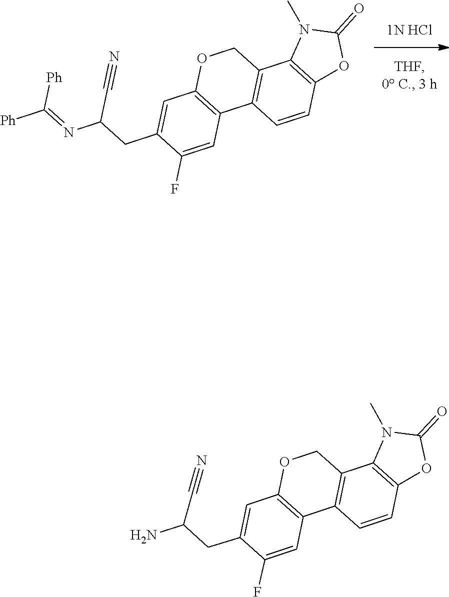

Into a 50 mL round-bottom flask were added 2-amino-3-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}propanenitrile (200 mg, 0.40 mmol, 1.0 equiv) and THF (15 mL), H₂O (1.5 mL), HCl (1 M) (0.75 mL) at room temperature. The resulting mixture was stirred for additional 3 h at room temperature. The mixture was basified to pH 12 with K₂CO₃(aq). The resulting mixture was extracted with EtOAc (3×20 mL). The combined organic layers were washed with brine (3×10 mL), dried over anhydrous Na₂SO₄. After filtration, the filtrate was concentrated under reduced pressure. The residue was purified by silica gel column chromatography, eluted with PE/THF (1:2) to afford 2-amino-3-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}propanenitrile (110 mg, 81%) as a light yellow solid. LCMS (ES, m/z): [M+H]⁺: 340.

### Step 11. Synthesis of tert-butyl 2-[(1-cyano-2-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}ethyl)carbamoyl]-1,4-oxazepane-4-carboxylate

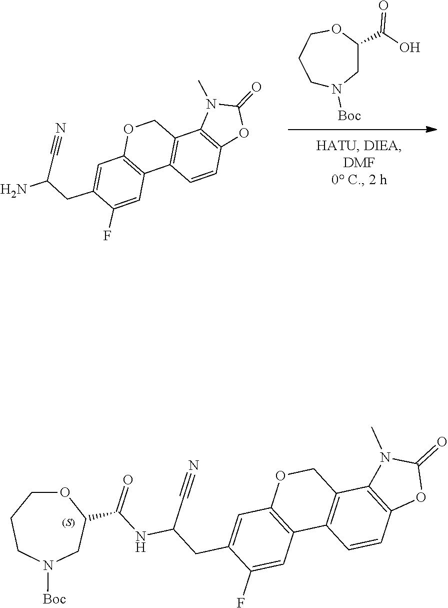

To a stirred solution of 2-amino-3-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}propanenitrile (110 mg, 0.32 mmol, 1.0 equiv), (2S)-4-(tert-butoxycarbonyl)-1,4-oxazepane-2-carboxylic acid (79 mg, 0.32 mmol, 1 equiv) and DIEA (125 mg, 0.97 mmol, 3.0 equiv) in DMF (5 mL) were added HATU (147 mg, 0.38 mmol, 1.2 equiv) in portions at 0° C. The resulting mixture was stirred for additional 3 h at 0° C. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/THF (1:1) to afford tert-butyl 2-[(1-cyano-2-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}ethyl)carbamoyl]-1,4-oxazepane-4-carboxylate (140 mg, 76.2%) as a light yellow oil. LCMS (ES, m/z): [M+H]⁺: 567.

### Step 12. Synthesis of N-(1-cyano-2-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}ethyl)-1,4-oxazepane-2-carboxamide

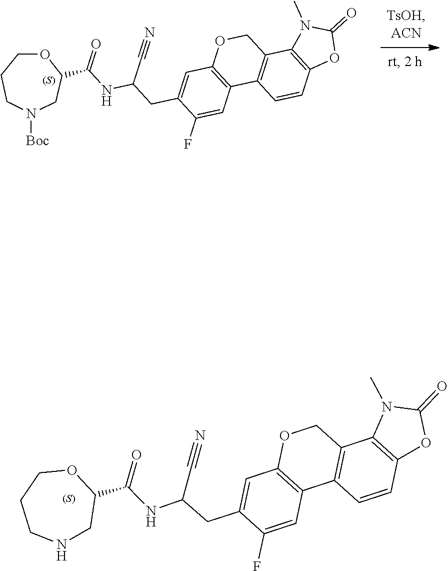

Into a 25 mL round-bottom flask were added tert-butyl 2-[(1-cyano-2-{4-fluoro-12-methyl-13-oxo-8,14-dioxa-12-azatetracyclo[8.7.0.0\hat{ }{2,7}0.0\hat{ }{11,15}]heptadeca-1(10),2(7),3,5,11(15),16-hexaen-5-yl}ethyl)carbamoyl]-1,4-oxazepane-4-carboxylate (140 mg, 0.24 mmol, 1.0 equiv), TsOH·H₂O (220 mg, 0.74 mmol, 3.0 equiv) and ACN (4 mL) at room temperature. The resulting mixture was stirred for additional 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound C2 (20 mg, 17.4%) as a white solid. LCMS (ES, m/z): [M+H]⁺: 467.1. ¹H NMR (400 MHz, DMSO-d₆): δ 8.65 (dd, J=11.4, 8.5 Hz, 1H), 7.72 (dd, J=10.8, 3.6 Hz, 1H), 7.63 (dd, J=8.5, 2.5 Hz, 1H), 7.36 (d, J=8.3 Hz, 1H), 6.99 (d, J=6.4 Hz, 1H), 5.49-5.40 (m, 2H), 5.06-4.92 (m, 1H), 4.02-3.79 (m, 2H), 3.77-3.66 (m, 1H), 3.51 (s, 3H), 3.19-2.97 (m, 3H), 2.84-2.50 (m, 3H), 1.82-1.61 (m, 2H).

### Example 54: Synthesis (2S)—N-(1-cyano-2-{2-fluoro-1′-methyl-8,9-dihydro-6H-spiro[indeno[5,6-c]chromene-10,4′-piperidin]-3-yl}ethyl)-1,4-oxazepane-2-carboxamide (Compound C3)

### Step 1. Synthesis of 6-chloro-1′-methyl-2,3-dihydrospiro[indene-1,4′-piperidine]-5-carbaldehyde and 5-chloro-1′-methyl-2,3-dihydrospiro[indene-1,4′-piperidine]-6-carbaldehyde

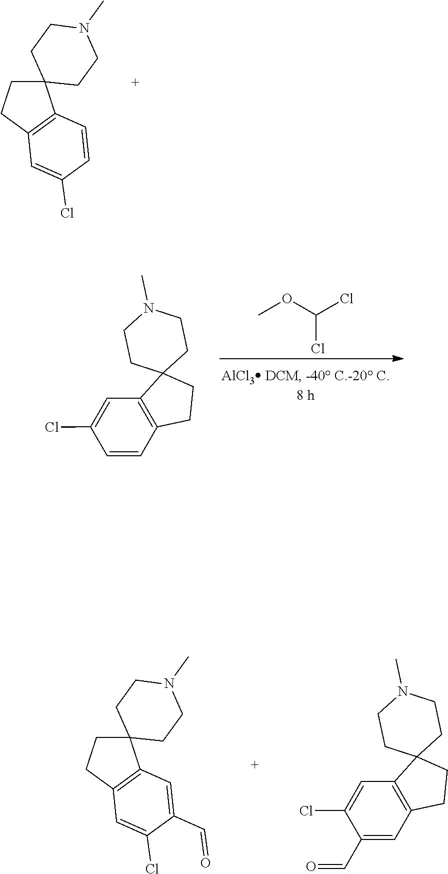

To a stirred mixture of 6-chloro-1′-methyl-2,3-dihydrospiro[indene-1,4′-piperidine] and 5-chloro-1′-methyl-2,3-dihydrospiro[indene-1,4′-piperidine](6.00 g, 25.450 mmol, 1.0 equiv) in DCM (120 mL) were added AlCl₃ (6.79 g, 50.900 mmol, 2.00 equiv) and dichloromethyl methyl ether (5.85 g, 50.900 mmol, 2.0 equiv) in portions at −40° C. under nitrogen atmosphere. The resulting mixture was stirred at −20° C. for 8 h under nitrogen atmosphere. The reaction was quenched with sat. NH₄Cl (aq.) at 0° C. The resulting mixture was extracted with CH₂Cl₂ (8×150 mL). The combined organic layers were washed with brine (100 mL), dried over anhydrous Na₂SO₄. After filtration, the filtrate was concentrated under reduced pressure. This resulted in 6-chloro-1′-methyl-2,3-dihydrospiro[indene-1,4′-piperidine]-5-carbaldehyde and 5-chloro-1′-methyl-2,3-dihydrospiro[indene-1,4′-piperidine]-6-carbaldehyde (6 g, 89.3%) as a light-yellow oil. LCMS (ES) [M+1]⁺ m/z: 264.

### Step 2. Synthesis of 6-chloro-1′-methyl-2,3-dihydrospiro[indene-1,4′-piperidin]-5-ylmethanol and 5-chloro-1′-methyl-2,3-dihydrospiro[indene-1,4′-piperidin]-6-ylmethanol

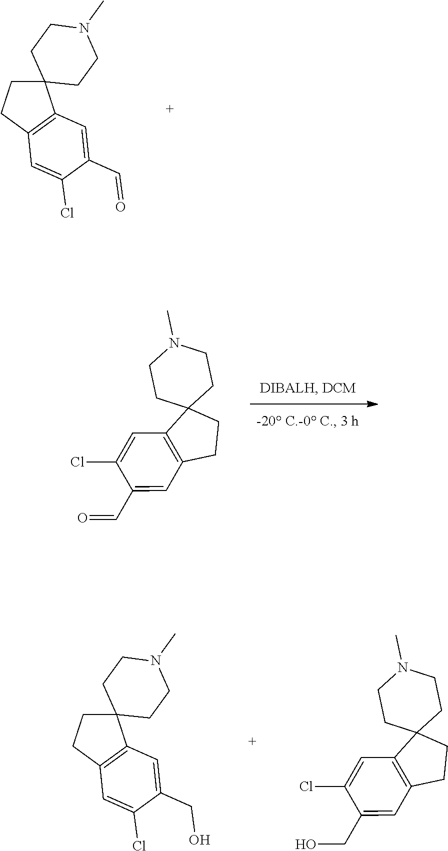 [SMILES: CN1CCC2(CCc3cc(Cl)c(C=O)cc32)CC1 ; CN1CCC2(CCc3cc(C=O)c(Cl)cc32)CC1 ; CN1CCC2(CCc3cc(Cl)c(CO)cc32)CC1 ; CN1CCC2(CCc3cc(CO)c(Cl)cc32)CC1]

To a stirred mixture of 6-chloro-1′-methyl-2,3-dihydrospiro[indene-1,4′-piperidine]-5-carbaldehyde and 5-chloro-1′-methyl-2,3-dihydrospiro[indene-1,4′-piperidine]-6-carbaldehyde (6.00 g, 22.747 mmol, 1.0 equiv) in DCM (120 mL) were added DIBALH (30 mL, 45.496 mmol, 2.0 equiv) dropwise at −20° C. under nitrogen atmosphere. The resulting mixture was stirred at 0° C. for 3 h under nitrogen atmosphere. The reaction was quenched with potassium sodium tartrate (aq.) at 0° C. The resulting mixture was extracted with CH₂C12 (8×150 mL). The combined organic layers were washed with brine (100 mL), dried over anhydrous Na₂SO₄. After filtration, the filtrate was concentrated under reduced pressure. The crude product was purified by Prep-HPLC with the following conditions (Column, Kinetex EVO C18 Column, 21.2*150.5 um; mobile phase, Water (0.1% TFA) and ACN (10% PhaseB up to 50% in 15 min); Detector, UV 254 nm. This resulted in 6-chloro-1′-methyl-2,3-dihydrospiro[indene-1,4′-piperidin]-5-ylmethanol (2.5 g, 41.35%) and 5-chloro-1′-methyl-2,3-dihydrospiro[indene-1,4′-piperidin]-6-ylmethanol (2.5 g, 41.3%) as a colorless oil. LCMS (ES) [M+1]⁺ m/z: 266.

### Step 3. Synthesis of 4-bromo-2-fluoro-5-hydroxybenzoic acid

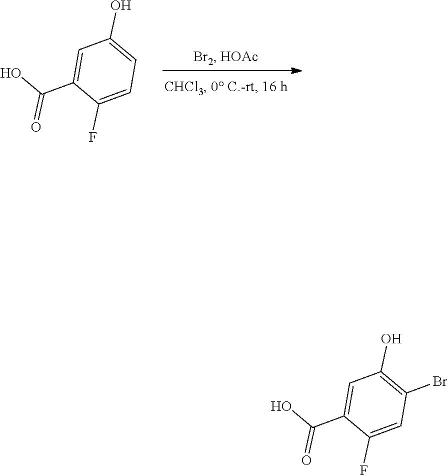 [SMILES: O=C(O)c1cc(O)ccc1F ; O=C(O)c1cc(O)c(Br)cc1F]

To a stirred solution of 2-fluoro-5-hydroxybenzoic acid (27 g, 172.953 mmol, 1.0 equiv) in CHCl₃ (300 mL) were added Br₂ (82.92 g, 518.859 mmol, 3.0 equiv) in HOAc (300 mL) dropwise at 0° C. under nitrogen atmosphere. The resulting mixture was stirred at room temperature for 16 h. The reaction was quenched with sat. sodium hyposulfite (aq.) at 0° C. The resulting mixture was extracted with CH₂Cl₂ (3×500 mL). The combined organic layers were washed with brine (300 mL), dried over anhydrous Na₂SO₄. After filtration, the filtrate was concentrated under reduced pressure. The residue was purified by silica gel column chromatography, eluted with PE/THF (6:1) to afford 4-bromo-2-fluoro-5-hydroxybenzoic acid (18 g, 44.2%) as a white solid. LCMS (ES) [M-1]⁻ m/z: 233.

### Step 4. Synthesis of methyl 4-bromo-2-fluoro-5-hydroxybenzoate

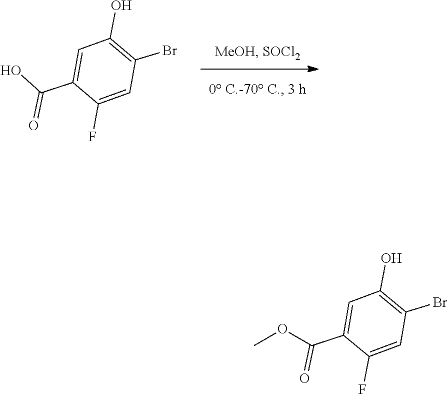

To a stirred solution of 4-bromo-2-fluoro-5-hydroxybenzoic acid (18 g, 76.593 mmol, 1.0 equiv) in MeOH (200 mL) was added SOCl₂ (27.33 g, 229.779 mmol, 3.0 equiv) dropwise at 0° C. under nitrogen atmosphere. The resulting mixture was stirred at 70° C. for 3 h. The mixture was allowed to cool down to room temperature. The resulting mixture was concentrated under reduced pressure. The residue was purified by silica gel column chromatography, eluted with PE/EA (9:1) to afford methyl 4-bromo-2-fluoro-5-hydroxybenzoate (16 g, 83.8%) as a white solid. LCMS (ES) [M-1]⁻ m/z: 247.

### Step 5. Synthesis of methyl 4-bromo-2-fluoro-5-[(4-methoxyphenyl)methoxy]benzoate

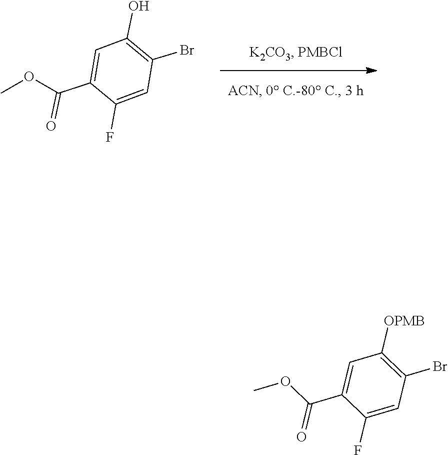

To a stirred solution of methyl 4-bromo-2-fluoro-5-hydroxybenzoate (5 g, 20.077 mmol, 1.0 equiv) and K₂CO₃ (5.55 g, 40.154 mmol, 2.0 equiv) in ACN (70 mL) were added PMBCl (3.46 g, 22.085 mmol, 1.1 equiv) in portions at 0° C. under nitrogen atmosphere. The resulting mixture was stirred at 80° C. for 3 h. The mixture was allowed to cool down to room temperature. The resulting mixture was diluted with water (100 mL). The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (15:1) to afford methyl 4-bromo-2-fluoro-5-[(4-methoxyphenyl)methoxy]benzoate (6 g, 80.9%) as a white solid.

### Step 6. Synthesis of methyl 2-fluoro-5-[(4-methoxyphenyl)methoxy]-4-(4,4,5,5-tetramethyl-1,3,2-dioxaborolan-2-yl)benzoate

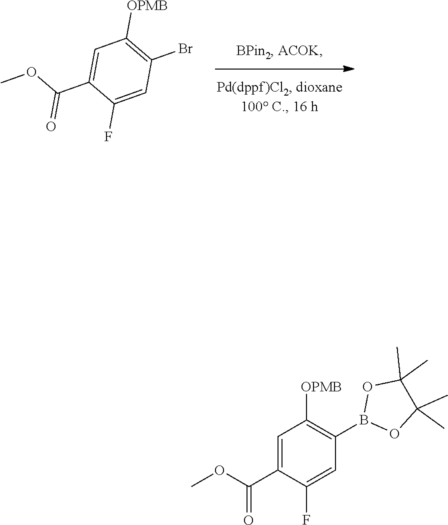

To a stirred solution of methyl 4-bromo-2-fluoro-5-[(4-methoxyphenyl)methoxy]benzoate (6 g, 16.252 mmol, 1.0 equiv) and bis(pinacolato)diboron (4.95 g, 19.502 mmol, 1.2 equiv) in dioxane (120 mL) were added KOAc (3.19 g, 32.504 mmol, 2.0 equiv) and Pd(dppf)Cl₂ (1.19 g, 1.625 mmol, 0.1 equiv). The resulting mixture was stirred at 100° C. for 16 h under nitrogen atmosphere. The mixture was allowed to cool down to room temperature. The resulting mixture was concentrated under reduced pressure. The residue was purified by silica gel column chromatography, eluted with PE/EA (9:1) to afford methyl 2-fluoro-5-[(4-methoxyphenyl)methoxy]-4-(4,4,5,5-tetramethyl-1,3,2-dioxaborolan-2-yl)benzoate (4.8 g, 70.9%) as a white solid. LCMS (ES) [M+1]⁺ m/z: 417.

### Step 7. Synthesis of methyl 2-fluoro-4-[5-(hydroxymethyl)-1′-methyl-2,3-dihydrospiro[indene-1,4′-piperidin]-6-yl]-5-[(4-methoxyphenyl)methoxy]benzoate

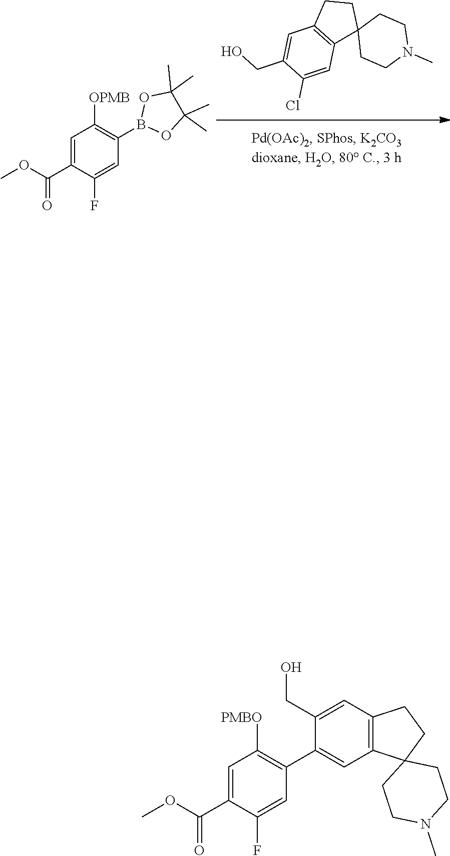

To a stirred solution of methyl 2-fluoro-5-[(4-methoxyphenyl)methoxy]-4-(4,4,5,5-tetramethyl-1,3,2-dioxaborolan-2-yl)benzoate (5.17 g, 12.417 mmol, 1.1 equiv) and 6-chloro-1′-methyl-2,3-dihydrospiro[indene-1,4′-piperidin]-5-ylmethanol (3 g, 11.288 mmol, 1.0 equiv) in dioxane (60 mL) and H₂O (6 mL) were added K₂CO₃ (3.12 g, 22.576 mmol, 2.0 equiv) and Sphos (0.93 g, 2.258 mmol, 0.2 equiv) and Pd(OAc)₂ (0.25 g, 1.129 mmol, 0.1 equiv). The resulting mixture was stirred at 80° C. for 3 h under nitrogen atmosphere. The mixture was allowed to cool down to room temperature. The resulting mixture was concentrated under reduced pressure. The residue was purified by silica gel column chromatography, eluted with CH₂Cl₂/MeOH (9:1) to afford methyl 2-fluoro-4-[5-(hydroxymethyl)-1′-methyl-2,3-dihydrospiro[indene-1,4′-piperidin]-6-yl]-5-[(4-methoxyphenyl)methoxy]benzoate (2.5 g, 42.6%) as an off-white solid. LCMS (ES) [M+1]⁺ m/z: 520.

### Step 8. Synthesis of methyl 2-fluoro-5-hydroxy-4-[5-(hydroxymethyl)-1′-methyl-2,3-dihydrospiro[indene-1,4′-piperidin]-6-yl]benzoate

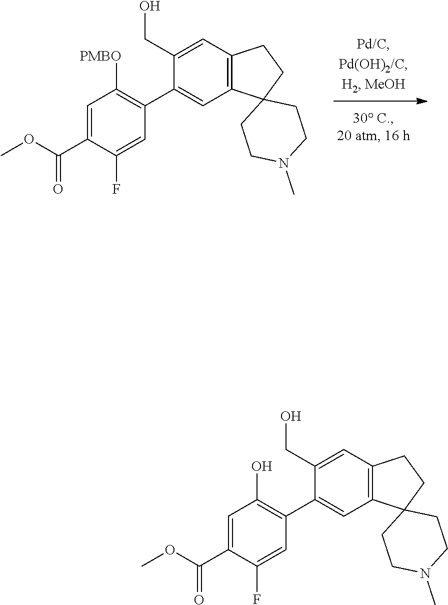

To a solution of methyl 2-fluoro-4-[5-(hydroxymethyl)-1′-methyl-2,3-dihydrospiro[indene-1,4′-piperidin]-6-yl]-5-[(4-methoxyphenyl)methoxy]benzoate (2.5 g, 4.811 mmol, 1.0 equiv) in MeOH (50 mL) was added Pd/C (500 mg, 10%) and Pd(OH)₂/C (500 mg, 20%) in a pressure tank. The mixture was hydrogenated at 30° C. under 20 atm of hydrogen pressure for 16 h, filtered through a Celite pad and concentrated under reduced pressure. The residue was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in methyl 2-fluoro-5-hydroxy-4-[5-(hydroxymethyl)-1′-methyl-2,3-dihydrospiro[indene-1,4′-piperidin]-6-yl]benzoate (1 g, 52.0%) as a white solid. LCMS (ES) [M+1]⁺ m/z: 400.

### Step 9. Synthesis of methyl 2-fluoro-1′-methyl-8,9-dihydro-6H-spiro[indeno[5,6-c]chromene-10,4′-piperidine]-3-carboxylate

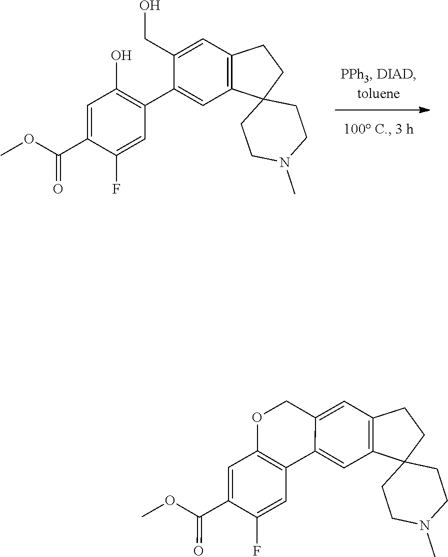 [SMILES: COC(=O)c1cc(O)c(-c2cc3c(cc2CO)CCC32CCN(C)CC2)cc1F ; COC(=O)c1cc2c(cc1F)-c1cc3c(cc1CO2)CCC31CCN(C)CC1]

To a stirred solution of methyl 2-fluoro-5-hydroxy-4-[5-(hydroxymethyl)-1′-methyl-2,3-dihydrospiro[indene-1,4′-piperidin]-6-yl]benzoate (1 g, 2.503 mmol, 1.0 equiv) in toluene (20 mL) were added PPh₃ (0.98 g, 3.755 mmol, 1.5 equiv) and DIAD (0.76 g, 3.755 mmol, 1.5 equiv) dropwise under nitrogen atmosphere. The resulting mixture was stirred at 100° C. for 3 h under nitrogen atmosphere. The mixture was allowed to cool down to room temperature. The resulting mixture was concentrated under reduced pressure. The residue was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in methyl 2-fluoro-1′-methyl-8,9-dihydro-6H-spiro[indeno[5,6-c]chromene-10,4′-piperidine]-3-carboxylate (580 mg, 60.7%) as a white solid. LCMS (ES) [M+1]⁺ m/z: 382.

### Step 10. Synthesis of 2-fluoro-1′-methyl-8,9-dihydro-6H-spiro[indeno[5,6-c]chromene-10,4′-piperidin]-3-ylmethanol

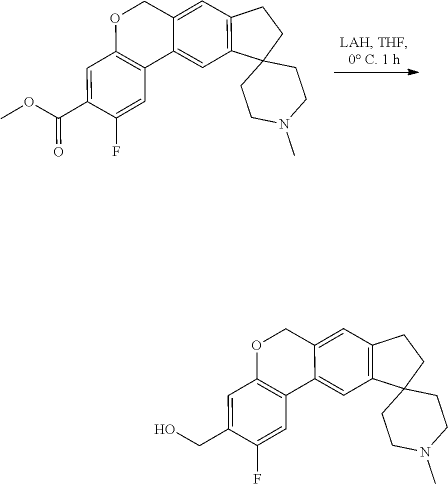 [SMILES: COC(=O)c1cc2c(cc1F)-c1cc3c(cc1CO2)CCC31CCN(C)CC1 ; CN1CCC2(CCc3cc4c(cc32)-c2cc(F)c(CO)cc2OC4)CC1]

To a stirred solution of methyl 2-fluoro-1′-methyl-8,9-dihydro-6H-spiro[indeno[5,6-c]chromene-10,4′-piperidine]-3-carboxylate (580 mg, 1.521 mmol, 1.0 equiv) in THF (15 mL) was added LAH (0.9 mL, 1.840 mmol, 1.2 equiv) dropwise at 0° C. under nitrogen atmosphere. The resulting mixture was stirred at 0° C. for 1 h under nitrogen atmosphere. The resulting mixture was diluted with THF (15 mL). The reaction was quenched with Na₂SO₄·10H₂O at 0° C. The resulting mixture was filtered, the filter cake was washed with THF (3×10 mL). The filtrate was concentrated under reduced pressure. This resulted in 2-fluoro-1′-methyl-8,9-dihydro-6H-spiro[indeno[5,6-c]chromene-10,4′-piperidin]-3-ylmethanol (450 mg, 83.7%) as a white solid. LCMS (ES) [M+1]⁺ m/z: 354.

### Step 11. Synthesis of 3-(bromomethyl)-2-fluoro-1′-methyl-8,9-dihydro-6H-spiro[indeno[5,6-c]chromene-10,4′-piperidine]

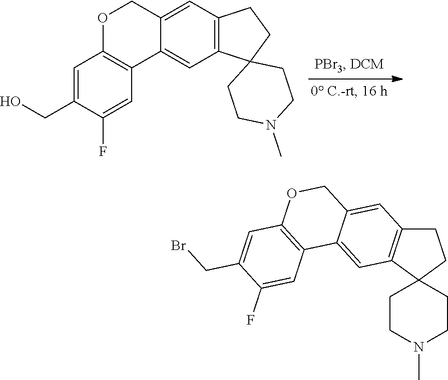

To a stirred solution of 2-fluoro-1′-methyl-8,9-dihydro-6H-spiro[indeno[5,6-c]chromene-10,4′-piperidin]-3-ylmethanol (450 mg, 1.273 mmol, 1.0 equiv) in DCM (10 mL) was added PBr₃ (241 mg, 0.891 mmol, 0.7 equiv) dropwise at 0° C. under nitrogen atmosphere. The resulting mixture was stirred at room temperature for 16 h. The resulting mixture was diluted with water (20 mL). The resulting mixture was extracted with CH₂Cl₂ (3×20 mL). The combined organic layers were dried over anhydrous Na₂SO₄. After filtration, the filtrate was concentrated under reduced pressure. This resulted in 3-(bromomethyl)-2-fluoro-1′-methyl-8,9-dihydro-6H-spiro[indeno[5,6-c]chromene-10,4′-piperidine](400 mg, 75.4%) as a white solid. LCMS (ES) [M+1]⁺ m/z: 416.

### Step 12. Synthesis of 2-[(diphenylmethylidene)amino]-3-{2-fluoro-1′-methyl-8,9-dihydro-6H-spiro[indeno[5,6-c]chromene-10,4′-piperidin]-3-yl}propanenitrile

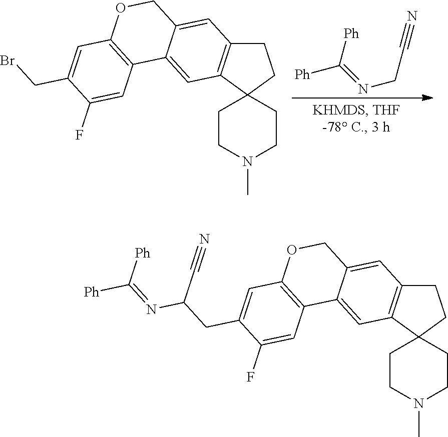

To a stirred solution of 2-[(diphenylmethylidene)amino]acetonitrile (253 mg, 1.153 mmol, 1.2 equiv) in THF (40 mL) was added KHMDS (1.4 mL, 1.442 mmol, 1.5 equiv) dropwise at −78° C. under nitrogen atmosphere. The resulting mixture was stirred at −78° C. for 0.5 h under nitrogen atmosphere. To the above mixture was added 3-(bromomethyl)-2-fluoro-1′-methyl-8,9-dihydro-6H-spiro[indeno[5,6-c]chromene-10,4′-piperidine](400 mg, 0.961 mmol, 1 equiv) in portions at −78° C. The resulting mixture was stirred at −78° C. for additional 3 h. The reaction was quenched with sat. NH₄Cl (aq.) at 0° C. The resulting mixture was diluted with water (50 mL). The resulting mixture was extracted with EtOAc (3×30 mL). The combined organic layers were washed with brine (30 mL), dried over anhydrous Na₂SO₄. After filtration, the filtrate was concentrated under reduced pressure. The residue was purified by silica gel column chromatography, eluted with PE/THF (1:3) to afford 2-[(diphenylmethylidene)amino]-3-{2-fluoro-1′-methyl-8,9-dihydro-6H-spiro[indeno[5,6-c]chromene-10,4′-piperidin]-3-yl}propanenitrile (200 mg, 37.4%) as a light yellow solid. LCMS (ES) [M+1]⁺ m/z: 556.

### Step 13. Synthesis of 2-amino-3-{2-fluoro-1′-methyl-8,9-dihydro-6H-spiro[indeno[5,6-c]chromene-10,4′-piperidin]-3-yl}propanenitrile

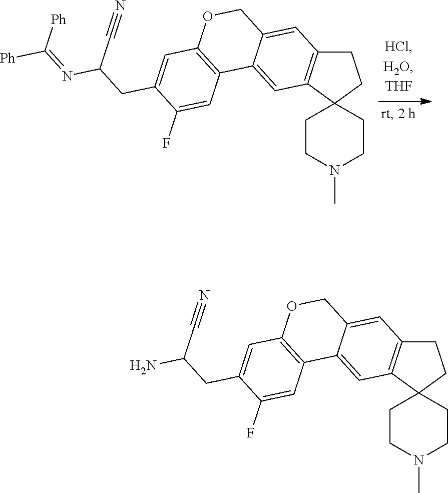 [SMILES: CN1CCC2(CCc3cc4c(cc32)-c2cc(F)c(CC(C#N)N=C(c3ccccc3)c3ccccc3)cc2OC4)CC1 ; CN1CCC2(CCc3cc4c(cc32)-c2cc(F)c(CC(N)C#N)cc2OC4)CC1]

To a stirred solution of 2-[(diphenylmethylidene)amino]-3-{2-fluoro-1′-methyl-8,9-dihydro-6H-spiro[indeno[5,6-c]chromene-10,4′-piperidin]-3-yl}propanenitrile (200 mg, 0.360 mmol, 1.0 equiv) in THF (10 mL) and H₂O (1 mL) was added HCl (1N) (0.5 mL). The resulting mixture was stirred at room temperature for 2 h. The resulting mixture was concentrated under reduced pressure. The resulting mixture was diluted with water (20 mL). The aqueous layer was extracted with EtOAc (2×10 mL). The aqueous layer was basified to pH 10 with NaOH (1 N) (aq.). The aqueous layer was extracted with CH₂Cl₂ (3×10 mL). The combined organic layers were washed with brine (10 mL), dried over anhydrous Na₂SO₄. After filtration, the filtrate was concentrated under reduced pressure. This resulted in 2-amino-3-{2-fluoro-1′-methyl-8,9-dihydro-6H-spiro[indeno[5,6-c]chromene-10,4′-piperidin]-3-yl}propanenitrile (80 mg, 56.7%) as a light yellow solid. LCMS (ES) [M+1]⁺ m/z: 392.

### Step 14. Synthesis of tert-butyl (2S)-2-[(1-cyano-2-{2-fluoro-1′-methyl-8,9-dihydro-6H-spiro[indeno[5,6-c]chromene-10,4′-piperidin]-3-yl}ethyl)carbamoyl]-1,4-oxazepane-4-carboxylate

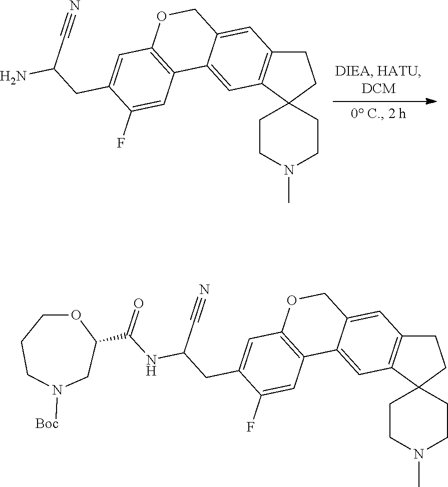 [SMILES: CN1CCC2(CCc3cc4c(cc32)-c2cc(F)c(CC(N)C#N)cc2OC4)CC1 ; CN1CCC2(CCc3cc4c(cc32)-c2cc(F)c(CC(C#N)NC(=O)[C@@H]3CN(C(=O)OC(C)(C)C)CCCO3)cc2OC4)CC1]

To a stirred solution of 2-amino-3-{2-fluoro-1′-methyl-8,9-dihydro-6H-spiro[indeno[5,6-c]chromene-10,4′-piperidin]-3-yl}propanenitrile (70 mg, 0.179 mmol, 1.0 equiv) and (2S)-4-(tert-butoxycarbonyl)-1,4-oxazepane-2-carboxylic acid (48 mg, 0.197 mmol, 1.1 equiv) in DCM (2 mL) were added DIEA (69 mg, 0.537 mmol, 3.0 equiv) and HATU (81 mg, 0.215 mmol, 1.2 equiv) in portions at 0° C. under nitrogen atmosphere. The resulting mixture was stirred at 0° C. for 2 h. The resulting mixture was concentrated under reduced pressure. The crude product was purified by Prep-HPLC with the following conditions (Column, XBridge Prep C18 OBD Column, 19*150 mm Sum; mobile phase, Water (10 MMOL/L NH₄HCO₃) and ACN (10% PhaseB up to 80% in 20 min); Detector, UV 254 nm. This resulted in tert-butyl (2S)-2-[(1-cyano-2-{2-fluoro-1′-methyl-8,9-dihydro-6H-spiro[indeno[5,6-c]chromene-10,4′-piperidin]-3-yl}ethyl)carbamoyl]-1,4-oxazepane-4-carboxylate (60 mg, 54.2%) as a white solid. LCMS (ES) [M+1]⁺ m/z: 619.

### Step 15. Synthesis of (2S)—N-(1-cyano-2-{2-fluoro-1′-methyl-8,9-dihydro-6H-spiro[indeno[5,6-c]chromene-10,4′-piperidin]-3-yl}ethyl)-1,4-oxazepane-2-carboxamide

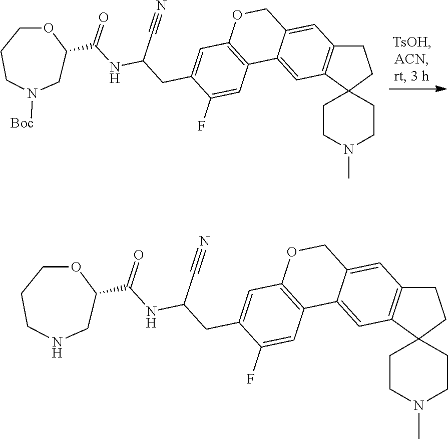 [SMILES: CN1CCC2(CCc3cc4c(cc32)-c2cc(F)c(CC(C#N)NC(=O)[C@@H]3CN(C(=O)OC(C)(C)C)CCCO3)cc2OC4)CC1 ; CN1CCC2(CCc3cc4c(cc32)-c2cc(F)c(CC(C#N)NC(=O)[C@@H]3CNCCCO3)cc2OC4)CC1]

To a stirred solution of tert-butyl (2S)-2-[(1-cyano-2-{2-fluoro-1′-methyl-8,9-dihydro-6H-spiro[indeno[5,6-c]chromene-10,4′-piperidin]-3-yl}ethyl)carbamoyl]-1,4-oxazepane-4-carboxylate (60 mg, 0.097 mmol, 1.0 equiv) in ACN (2 mL) was added TsOH (50 mg, 0.291 mmol, 3.0 equiv). The resulting mixture was stirred at room temperature for 3 h. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound C3 (20 mg, 39.7%) as a white solid. LCMS (ES) [M+1]⁺ m/z: 519. ¹H NMR (300 MHz, DMSO-d6) δ 8.66 (t, J=8.8 Hz, 1H), 7.83 (dd, J=10.8, 2.4 Hz, 1H), 7.71 (s, 1H), 7.09 (s, 1H), 6.93 (d, J=6.5 Hz, 1H), 5.04 (s, 2H), 4.96 (dt, J=16.1, 7.7 Hz, 1H), 4.03-3.64 (m, 3H), 3.27-2.97 (m, 3H), 2.85 (t, J=7.3 Hz, 2H), 2.81-2.53 (m, 5H), 2.22 (s, 3H), 2.14-1.90 (m, 6H), 1.77-1.70 (m, 2H), 1.42 (d, J=11.0 Hz, 2H).

### Example 55: Synthesis (2S)—N-(1-cyano-2-{8-fluoro-1′-methyl-2,4-dihydro-1H-spiro[indeno[4,5-c]chromene-3,4′-piperidin]-7-yl}ethyl)-1,4-oxazepane-2-carboxamide (Compound C4)

### Step 1. Synthesis of tert-butyl 7-bromospiro[indene-1,4′-piperidine]-1′-carboxylate

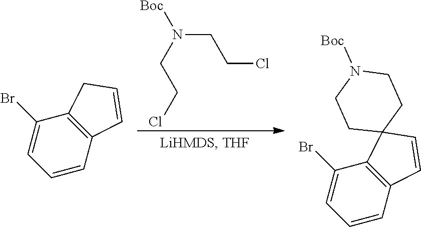

To a stirred solution of 4-bromo-3H-indene (10 g, 51.26 mmol, 1.00 equiv) in THF (150 mL) was added LiHMDS (21 g, 128.17 mmol, 2.50 equiv) dropwise at 0° C. under nitrogen atmosphere. After the reaction stirred at 0 for 1 h. tert-butyl N,N-bis(2-chloroethyl)carbamate (14.9 g, 61.52 mmol, 1.20 equiv) was added to the reaction. The resulting mixture was stirred for 16 h at 0° C. to room temperature under nitrogen atmosphere. The reaction was quenched by the addition of sat. NH₄Cl (aq.) (100 mL) at 0° C. The resulting mixture was extracted with EtOAc (3×250 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (50:1 to 1:1) followed by Pre-HPLC (with the following conditions: column, C18 silica gel; mobile phase, MeCN in Water (0.1% NH₃·H₂O), 10% to 85% gradient in 28 min; detector, UV 254 nm). to afford tert-butyl 7-bromospiro[indene-1,4′-piperidine]-1′-carboxylate (8.9 g, 47.66%) as a light yellow oil. LCMS (ES, m/z): [M+H]⁺: 364/366.

### Step 2. Synthesis of 1′-tert-butyl 7-methyl spiro[indene-1,4′-piperidine]-1′,7-dicarboxylate

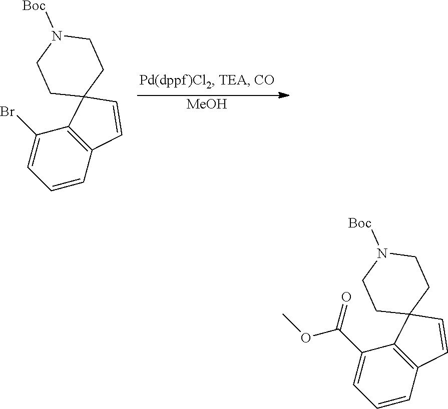 [SMILES: "MeOH" ; CC(C)(C)OC(=O)N1CCC2(C=Cc3cccc(Br)c32)CC1 ; COC(=O)c1cccc2c1C1(C=C2)CCN(C(=O)OC(C)(C)C)CC1]

To a solution of tert-butyl 7-bromospiro[indene-1,4′-piperidine]-1′-carboxylate (4.0 g, 10.98 mmol, 1.00 equiv) in MeOH (100 mL) were added TEA (5.5 g, 54.90 mmol, 5.00 equiv) and Pd(dppf)Cl₂·CH₂Cl₂ (1.79 g, 2.19 mmol, 0.20 equiv) in a pressure tank. The mixture was purged with CO for 3 times and then was pressurized to 30 atm of CO. The resulting mixture was stirred at 130° C. for 16 h. The reaction mixture was concentrated under reduced pressure. The residue was purified by silica gel column chromatography, eluted with PE/THF (1:1) to afford 1′-tert-butyl 7-methyl spiro[indene-1,4′-piperidine]-1′,7-dicarboxylate (2.7 g, 71.60%) as a light yellow semi solid. LCMS (ES, m/z): [M+H]⁺: 344.

### Step 3. Synthesis of 1′-tert-butyl 7-methyl 2,3-dihydrospiro[indene-1,4′-piperidine]-1′,7-dicarboxylate

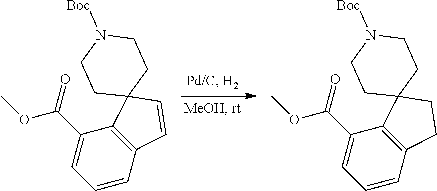 [SMILES: COC(=O)c1cccc2c1C1(C=C2)CCN(C(=O)OC(C)(C)C)CC1 ; COC(=O)c1cccc2c1C1(CC2)CCN(C(=O)OC(C)(C)C)CC1]

In a 100 mL round bottom flask, to a solution of 1′-tert-butyl 7-methyl spiro[indene-1,4′-piperidine]-1′,7-dicarboxylate (1.6 g, 4.66 mmol, 1.00 equiv) in MeOH (20 mL) was added Pd/C (200 mg, 0.09 mmol, 0.02 equiv, 5%) under nitrogen atmosphere. The reaction mixture was stirred for 5 h under hydrogen atmosphere using a hydrogen balloon. The resulting reaction was filtered through a Celite pad, the filtrate was concentrated under reduced pressure. The resulting crude product was purified by flash chromatography on silica gel eluted with PE/THF (100:1 to 20:1) to afford 1-tert-butyl 7-methyl 2,3-dihydrospiro[indene-1,4′-piperidine]-1′,7-dicarboxylate (1.1 g, 68.35%) as an off white solid. LCMS (ES, m/z): [M+H]⁺: 346.

### Step 4. Synthesis of tert-butyl 7-(hydroxymethyl)-2,3-dihydrospiro[indene-1,4′-piperidine]-1′-carboxylate

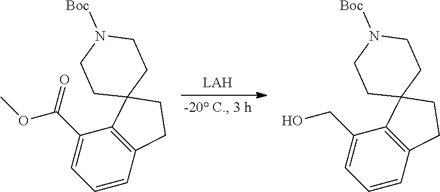 [SMILES: COC(=O)c1cccc2c1C1(CC2)CCN(C(=O)OC(C)(C)C)CC1 ; CC(C)(C)OC(=O)N1CCC2(CCc3cccc(CO)c32)CC1]

To a stirred solution of 1-tert-butyl 7-methyl 2,3-dihydrospiro[indene-1,4′-piperidine]-1′,7-dicarboxylate (1.1 g, 3.18 mmol, 1.00 equiv) in DCM (20 mL, 235.49 mmol, 49.83 equiv) was added DIAD (1.15 g, 5.67 mmol, 1.20 equiv) in THF (20 mL) was added LAH (3.82 mL, 3.82 mmol, 1.20 equiv) dropwise at −20° C. under nitrogen atmosphere. The resulting mixture was stirred for 2 h at −20° C. to 0° C. under nitrogen atmosphere. The resulting mixture was quenched with Na₂SO₄·10H₂O. The resulting mixture was filtered, the filtrate was concentrated under reduced pressure. The residue was purified by silica gel column chromatography, eluted with PE/THF (1:1) to afford tert-butyl 7-(hydroxymethyl)-2,3-dihydrospiro[indene-1,4′-piperidine]-1′-carboxylate (850 mg, 84.09%) as an off white solid. LCMS (ES, m/z): [M+H]⁺: 318.

### Step 5. Synthesis of tert-butyl 7-[2-bromo-4-fluoro-5-(methoxycarbonyl)phenoxymethyl]-2,3-dihydrospiro[indene-1,4′-piperidine]-1′-carboxylate

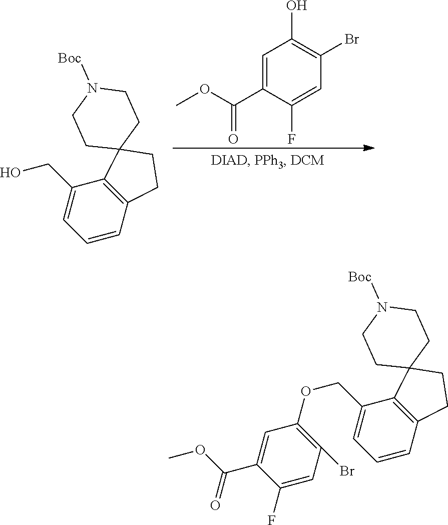

To a stirred solution of tert-butyl 7-(hydroxymethyl)-2,3-dihydrospiro[indene-1,4′-piperidine]-1′-carboxylate (1.5 g, 4.72 mmol, 1.00 equiv), methyl 4-bromo-2-fluorobenzoate (1.1 g, 4.72 mmol, 1.00 equiv) and PPh₃ (1.55 g, 5.90 mmol, 1.25 equiv) in DCM (20 mL) was added DIAD (1.15 g, 5.67 mmol, 1.20 equiv) in DCM (3 mL) dropwise at 0° C. under nitrogen atmosphere. The resulting mixture was stirred for 16 h at room temperature under nitrogen atmosphere. The resulting mixture was concentrated under reduced pressure. The residue was purified by silica gel column chromatography, eluted with PE/THF (1:1) to afford tert-butyl 7-[2-bromo-4-fluoro-5-(methoxycarbonyl)phenoxymethyl]-2,3-dihydrospiro[indene-1,4′-piperidine]-1′-carboxylate (1.3 g, 50.16%) as a light yellow semi-solid. LCMS (ES, m/z): [M+H]⁺: 548/550.

### Step 6. Synthesis of 1′-tert-butyl 7-methyl 8-fluoro-2,4-dihydro-1H-spiro[indeno[4,5-c]chromene-3,4′-piperidine]-1′,7-dicarboxylate

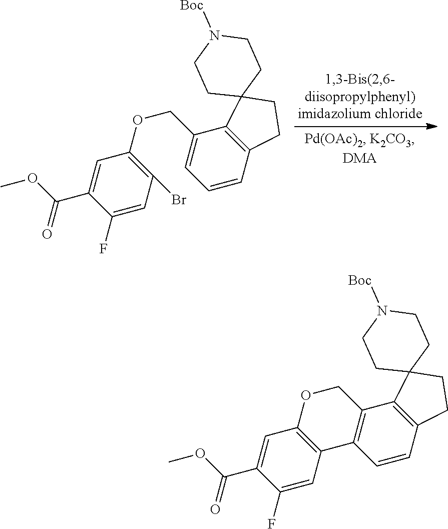 [SMILES: COC(=O)c1cc(OCc2cccc3c2C2(CC3)CCN(C(=O)OC(C)(C)C)CC2)c(Br)cc1F ; COC(=O)c1cc2c(cc1F)-c1ccc3c(c1CO2)C1(CC3)CCN(C(=O)OC(C)(C)C)CC1]

To a stirred solution of tert-butyl 7-[2-bromo-4-fluoro-5-(methoxycarbonyl)phenoxymethyl]-2,3-dihydrospiro[indene-1,4′-piperidine]-1′-carboxylate (1.0 g, 1.82 mmol, 1.00 equiv) and K₂CO₃ (0.50 g, 3.64 mmol, 2.00 equiv) in DMA (10 mL) was added Pd(OAc)₂ (40 mg, 0.18 mmol, 0.10 equiv) and 1,3-bis(2,6-diisopropylphenyl imidazolium chloride (0.16 g, 0.365 mmol, 0.20 equiv) under Ar atmosphere. The resulting mixture was stirred for 5 h at 130° C. under Ar atmosphere. The resulting mixture was cooled to room temperature and quenched with saturated NH₄Cl (10 mL), extracted with EtOAc (3×50 mL). The combined organic layers were washed with brine (50 mL), dried over anhydrous Na₂SO₄ and concentrated. The resulting crude product was purified by flash chromatography on silica gel eluted with PE/THF (100:1 to 1:1) to afford 1′-tert-butyl 7-methyl 8-fluoro-2,4-dihydro-1H-spiro[indeno[4,5-c]chromene-3,4′-piperidine]-1′,7-dicarboxylate (520 mg, 61.00%) as a light yellow solid. LCMS (ES, m/z): [M+H]⁺: 468.

### Step 7. Synthesis of 8-fluoro-1′-methyl-2,4-dihydro-1H-spiro[indeno[4,5-c]chromene-3,4′-piperidin]-7-ylmethanol

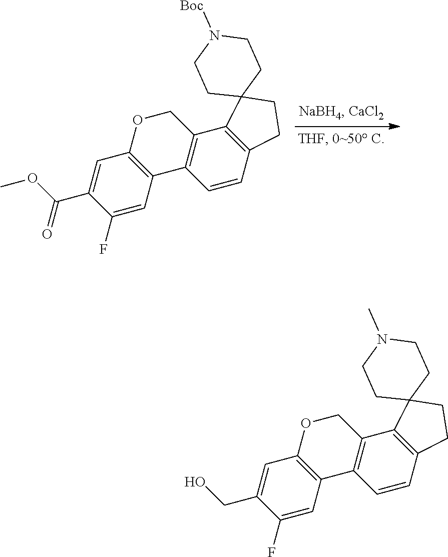 [SMILES: COC(=O)c1cc2c(cc1F)-c1ccc3c(c1CO2)C1(CC3)CCN(C(=O)OC(C)(C)C)CC1 ; CN1CCC2(CCc3ccc4c(c32)COc2cc(CO)c(F)cc2-4)CC1]

To a stirred solution of 1′-tert-butyl 7-methyl 8-fluoro-2,4-dihydro-1H-spiro[indeno[4,5-c]chromene-3,4′-piperidine]-1′,7-dicarboxylate (700 mg, 1.49 mmol, 1.00 equiv) in THF (10 mL) was added LAH (142 mg, 3.74 mmol, 2.50 equiv) in portions at 0° C. The resulting mixture was stirred for 2 h at 0° C. to 50° C. The reaction was quenched by the addition of Na₂SO₄·10H₂O (3 g) at 0° C. The resulting mixture was diluted with THF (50 mL) and filtered. The filtrate was concentrated under reduced pressure. The residue was purified by silica gel column chromatography, eluted with PE/EA (10:1 1:10) to afford 8-fluoro-1′-methyl-2,4-dihydro-1H-spiro[indeno[4,5-c]chromene-3,4′-piperidin]-7-ylmethanol (375 mg, 70.87%) as an off white solid. LCMS (ES, m/z): [M+H]⁺: 354.

### Step 8. Synthesis of 7-(bromomethyl)-8-fluoro-1′-methyl-2,4-dihydro-1H-spiro[indeno[4,5-c]chromene-3,4′-piperidine]

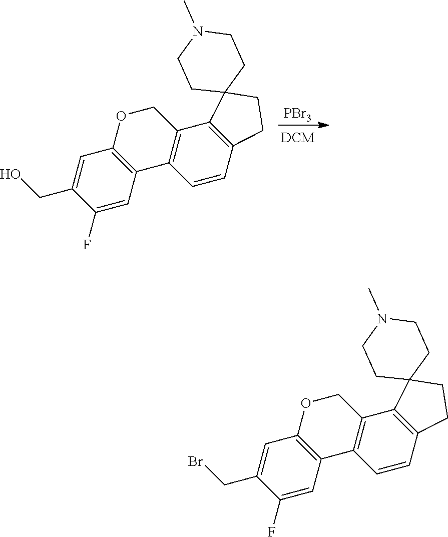

To a stirred solution of 8-fluoro-1′-methyl-2,4-dihydro-1H-spiro[indeno[4,5-c]chromene-3,4′-piperidin]-7-ylmethanol (200 mg, 0.57 mmol, 1.00 equiv) in DCM (10 mL) was added phosphorus tribromide (91 mg, 0.34 mmol, 0.60 equiv) dropwise at 0° C. under nitrogen atmosphere. The resulting mixture was stirred for 2 h at 0° C. to room temperature. The resulting mixture was quenched with saturated NaHCO₃ (2N, 5 mL), extracted with DCM (3×30 mL). The combined organic layers were washed with brine (50 mL), dried over anhydrous Na₂SO₄ and concentrated to give 7-(bromomethyl)-8-fluoro-1′-methyl-2,4-dihydro-1H-spiro[indeno[4,5-c]chromene-3,4′-piperidine](210 mg, 89.14%) as an off white solid. LCMS (ES, m/z): [M+H]⁺: 416/418.

### Step 9. Synthesis of 2-[(diphenylmethylidene)amino]-3-{8-fluoro-1′-methyl-2,4-dihydro-1H-spiro[indeno[4,5-c]chromene-3,4′-piperidin]-7-yl}propanenitrile

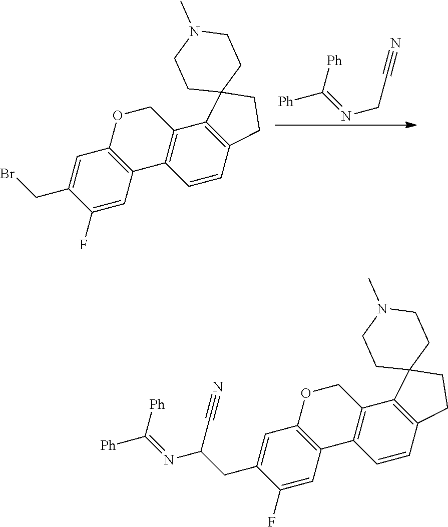

To a stirred solution of 2-[(diphenylmethylidene)amino]acetonitrile (127 mg, 0.58 mmol, 1.20 equiv) in THF (8 mL) was added KHMDS (0.72 mL, 0.72 mmol, 1.50 equiv) dropwise at 0° C. under nitrogen atmosphere. After the reaction stirred at 0 for 1 h. 7-(bromomethyl)-8-fluoro-1′-methyl-2,4-dihydro-1H-spiro[indeno[4,5-c]chromene-3,4′-piperidine](200 mg, 0.48 mmol, 1.00 equiv) in THF (2 mL) was added to the reaction dropwise. The resulting mixture was stirred for 5 h at 0° C. to room temperature under nitrogen atmosphere. The reaction was quenched by the addition of sat. NH₄C1 (aq.) (5 mL) at 0° C. The resulting mixture was extracted with EtOAc (3×50 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (50:1 to 1:1) to give 2-[(diphenylmethylidene)amino]-3-{8-fluoro-1′-methyl-2,4-dihydro-1H-spiro[indeno[4,5-c]chromene-3,4′-piperidin]-7-yl}propanenitrile (63 mg, 23.60%) as a light yellow solid. LCMS (ES, m/z): [M+H]⁺: 556.

### Step 10. Synthesis of 2-amino-3-{8-fluoro-1′-methyl-2,4-dihydro-1H-spiro[indeno[4,5-c]chromene-3,4′-piperidin]-7-yl}propanenitrile

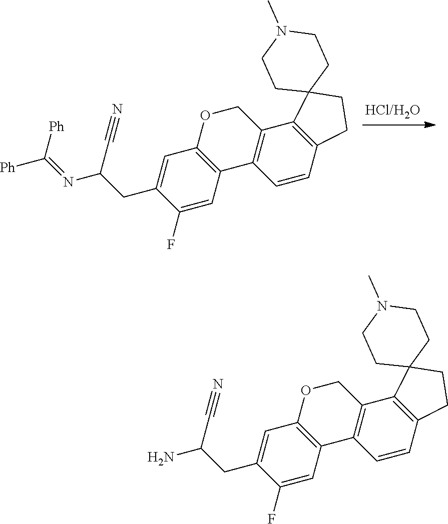 [SMILES: CN1CCC2(CCc3ccc4c(c32)COc2cc(CC(C#N)N=C(c3ccccc3)c3ccccc3)c(F)cc2-4)CC1 ; CN1CCC2(CCc3ccc4c(c32)COc2cc(CC(N)C#N)c(F)cc2-4)CC1]

To a solution of 2-[(diphenylmethylidene)amino]-3-{8-fluoro-1′-methyl-2,4-dihydro-1H-spiro[indeno[4,5-c]chromene-3,4′-piperidin]-7-yl}propanenitrile (60 mg, 0.11 mmol, 1.00 equiv) in THF (3 mL) was added HCl (0.1 mL, 0.100 mmol, 0.93 equiv) at room temperature. The resulting mixture was stirred for additional 3 h at room temperature. The mixture was basified to pH 12 with NaOH. The resulting mixture was extracted with EtOAc (3×25 mL). The combined organic layers were washed with brine (10 mL), dried over anhydrous Na₂SO₄. After filtration, the filtrate was concentrated under reduced pressure. The residue was purified by silica gel column chromatography, eluted with PE/THF (1:2) to afford 2-amino-3-{8-fluoro-1′-methyl-2,4-dihydro-1H-spiro[indeno[4,5-c]chromene-3,4′-piperidin]-7-yl}propanenitrile (29 mg, 68.61%) as an off white solid. LCMS (ES, m/z): [M+H]⁺: 392.

### Step 11. Synthesis of tert-butyl (2S)-2-[(1-cyano-2-{8-fluoro-1′-methyl-2,4-dihydro-1H-spiro[indeno[4,5-c]chromene-3,4′-piperidin]-7-yl}ethyl)carbamoyl]-1,4-oxazepane-4-carboxylate

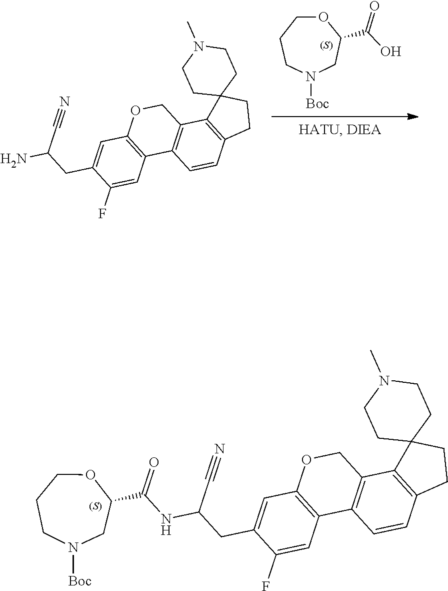

To a stirred solution of 2-amino-3-{8-fluoro-1′-methyl-2,4-dihydro-1H-spiro[indeno[4,5-c]chromene-3,4′-piperidin]-7-yl}propanenitrile (26 mg, 0.07 mmol, 1.00 equiv) and (2S)-4-(tert-butoxycarbonyl)-1,4-oxazepane-2-carboxylic acid (18 mg, 0.07 mmol, 1.10 equiv) in DCM (2 mL) were added DIEA (21 mg, 0.16 mmol, 2.50 equiv) and HATU (30 mg, 0.08 mmol, 1.20 equiv) in portions at 0° C. The resulting mixture was stirred for 2 h at 0° C. The resulting mixture was concentrated under reduced pressure. The residue was purified by Pre-TLC (eluted with DCM:MeOH=10:1) to afford tert-butyl (2S)-2-[(1-cyano-2-{8-fluoro-1′-methyl-2,4-dihydro-1H-spiro[indeno[4,5-c]chromene-3,4′-piperidin]-7-yl}ethyl)carbamoyl]-1,4-oxazepane-4-carboxylate (33 mg, 80.31%) as a light yellow semi-solid. LCMS (ES, m/z): [M+H]⁺: 619.

### Step 12. Synthesis of (2S)—N-(1-cyano-2-{8-fluoro-1′-methyl-2,4-dihydro-1H-spiro[indeno[4,5-c]chromene-3,4′-piperidin]-7-yl}ethyl)-1,4-oxazepane-2-carboxamide

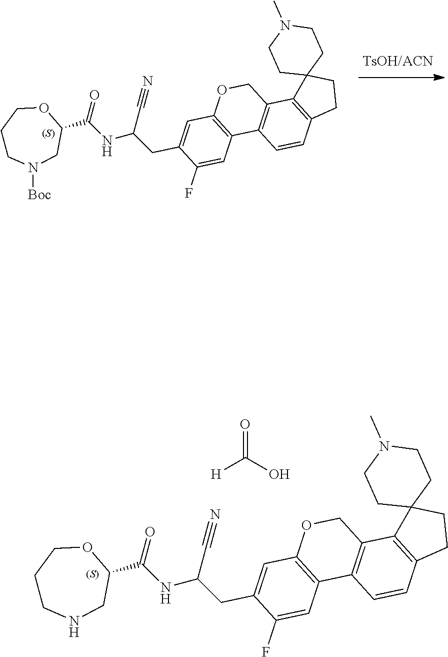

To a solution of tert-butyl (2S)-2-[(1-cyano-2-{8-fluoro-1′-methyl-2,4-dihydro-1H-spiro[indeno[4,5-c]chromene-3,4′-piperidin]-7-yl}ethyl)carbamoyl]-1,4-oxazepane-4-carboxylate (33 mg, 0.05 mmol, 1.00 equiv) in ACN (2 mL) was added TsOH·H₂O (30 mg, 0.16 mmol, 3.00 equiv) at 0° C. The resulting mixture was stirred for 3 h at 0° C. to room temperature. The reaction mixture was purified by Pre-HPLC with the following conditions: Column: XBridge Prep C18 Column, 19*250 mm, 5 m; Mobile Phase A: Water (0.1% NH₃·H₂O), Mobile Phase B: ACN; Flow rate: 25 mL/min mL/min; Gradient: 50% B to 70% B in 10 min; Wave Length: 254 nm/220 nm nm; RT1(min): 7.5. This resulted in Compound C4 (10 mg, 36.15%) as a white solid. LCMS (ES, m/z): [M+H]⁺: 519.3. ¹H NMR (400 MHz, DMSO-d₆) δ 8.67 (t, J=9.0 Hz, 1H), 8.22 (s, HCOOH), 7.73-7.55 (m, 2H), 7.25 (d, J=7.8 Hz, 1H), 6.98 (d, J=6.4 Hz, 1H), 5.26-5.24 (m, 2H), 5.02-4.98 (m, 1H), 3.99-3.96 (m, 1H), 3.92-3.79 (m, 1H), 3.73 (t, J=9.1 Hz, 1H), 3.22-3.19 (m, 1H), 3.18-3.08 (m, 4H), 2.89-2.86 (m, 2H), 2.76 (dd, J=18.2, 9.1 Hz, 2H), 2.71-2.60 (m, 1H), 2.25 (s, 3H), 2.20-1.95 (m, 6H), 1.82-1.67 (m, 2H), 1.52 (d, J=12.3 Hz, 2H). ¹⁹F NMR (376 MHz, DMSO-d₆) δ −125.49 (d, J=20.2 Hz).

### Example 56: Synthesis of (2S)—N-[(1S)-1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl]-1,4-oxazocane-2-carboxamide (Compound C5) and (2S)—N-[(1R)-1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl]-1,4-oxazocane-2-carboxamide (Compound C6)

### Step 1. Synthesis of tert-butyl (2S)-2-[(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl)carbamoyl]-1,4-oxazocane-4-carboxylate

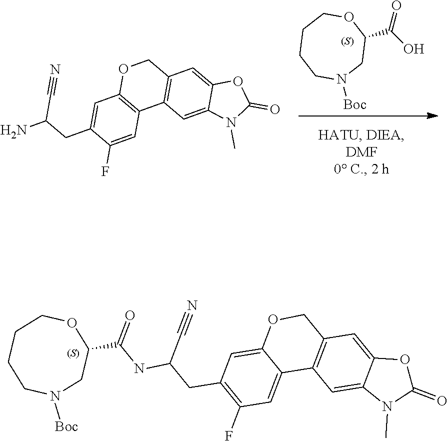

To a stirred mixture of 2-amino-3-{4-fluoro-15-methyl-14-oxo-8,13-dioxa-15-azatetracyclo[8.7.0.0\hat{ }{2,7}0.0\hat{ }{12,16}]heptadeca-1(10),2(7),3,5,11,16-hexaen-5-yl}propanenitrile (200 mg, 0.589 mmol, 1 equiv), (2S)-4-(tert-butoxycarbonyl)-1,4-oxazocane-2-carboxylic acid (152.83 mg, 0.589 mmol, 1 equiv) and DIEA (152.36 mg, 1.178 mmol, 2 equiv) in DMF (10 mL) were added HATU (268.93 mg, 0.707 mmol, 1.2 equiv) at 0° C. The resulting mixture was stirred for additional 36 h at room temperature. The residue was purified by silica gel column chromatography, eluted with PE/THF (1:1) to afford tert-butyl (2S)-2-[(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl)carbamoyl]-1,4-oxazocane-4-carboxylate (300 mg, 87.66%) as a light yellow oil. LCMS (ES, m/z): [M+H]⁺: 581.

### Step 2. Synthesis of 2S)—N-[(1S)-1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl]-1,4-oxazocane-2-carboxamide and (2S)—N-[(1R)-1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl]-1,4-oxazocane-2-carboxamide

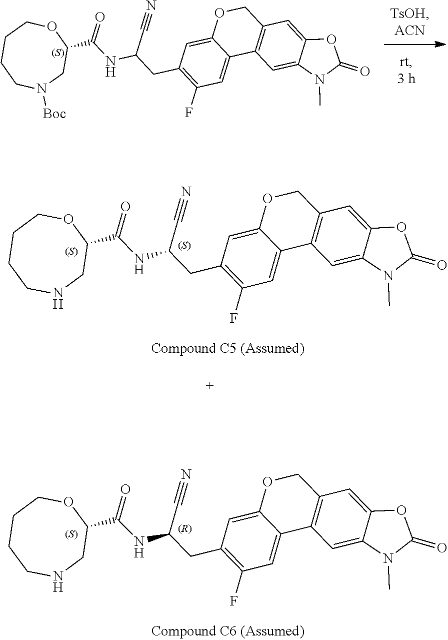

Into a 50 mL round-bottom flask were added tert-butyl (2S)-2-[(1-cyano-2-{4-fluoro-15-methyl-14-oxo-8,13-dioxa-15-azatetracyclo[8.7.0.0\hat{ }{2,7}0.0\hat{ }{12,16}]heptadeca-1(10),2,4,6,11,16-hexaen-5-yl}ethyl)carbamoyl]-1,4-oxazocane-4-carboxylate (300 mg, 0.51 mmol, 1 equiv), TsOH·H₂O (294 mg, 1.55 mmol, 3 equiv) and ACN (10 mL) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 60% gradient in 12 min; detector, UV 254 nm. The product was purified by Prep-CHIRAL-HPLC with the following conditions: Column: XA-CHIRALPAK IC, 2*25 cm, 5 m; Mobile Phase A: Hex:DCM=1:1-HPLC, Mobile Phase B: EtOH (0.1% DEA); Flow rate: 25 mL/min; Gradient: isocratic 50; Wave Length: 254 nm; RT1(min): 10.2; RT2(min): 14.2; Sample Solvent: EtOH:DCM=1:1-HPLC; Injection Volume: 2.5 mL; Number Of Runs: 5. This resulted in Compound C5 (20 mg, 16.11%) and Compound C6 (20 mg, 16.11%) as a white solid. LCMS (ES, m/z): [M+H]⁺: 481.

Compound C5: ¹H NMR (300 MHz, DMSO-d₆): δ 8.72 (d, J=8.7 Hz, 1H), 7.86-7.78 (m, 2H), 7.32 (s, 1H), 6.96 (d, J=6.5 Hz, 1H), 5.11 (s, 2H), 5.06-4.98 (m, 1H), 4.03-3.85 (m, 2H), 3.70-3.62 (m, 1H), 3.40 (s, 3H), 3.29-3.19 (m, 1H), 3.19-3.09 (m, 1H), 2.97 (t, J=13.2 Hz, 2H), 2.70-2.57 (m, 1H), 2.34-2.23 (m, 1H), 1.96-1.82 (m, 1H), 1.61-1.52 (m, 3H).

Compound C6: ¹H NMR (300 MHz, DMSO-d₆): δ 8.71 (d, J=8.5 Hz, 1H), 7.86-7.78 (m, 2H), 7.32 (s, 1H), 6.99 (d, J=6.5 Hz, 1H), 5.12 (s, 2H), 4.95 (q, J=8.1 Hz, 1H), 3.97 (t, J=9.9 Hz, 1H), 3.78 (dd, J=9.3, 2.6 Hz, 1H), 3.66-3.57 (m, 1H), 3.40 (s, 3H), 3.21 (q, J=10.0, 8.2 Hz, 2H), 3.16-3.05 (m, 1H), 3.03-2.95 (m, 1H), 2.72-2.57 (m, 1H), 2.47-2.37 (m, 1H), 1.91-1.87 (m, 1H), 1.55-1.50 (m, 3H).

### Example 57: Synthesis of (2S)—N-[(1S)-2-{4-[5-(5-amino-1,3,4-thiadiazol-2-yl)furan-2-yl]phenyl}-1-cyanoethyl]-1,4-oxazepane-2-carboxamide (Compound D1)

### Step 1. Synthesis of (E)-[(5-bromofuran-2-yl)methylidene]aminothiourea

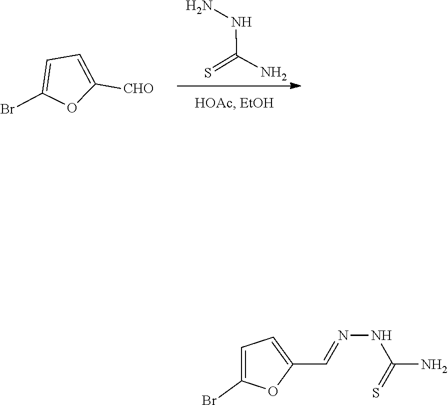 [SMILES: O=Cc1ccc(Br)o1 ; NNC(N)=S ; NC(=S)N/N=C/c1ccc(Br)o1]

To a stirred solution of 5-bromofuran-2-carbaldehyde (3 g, 17.14 mmol, 1 equiv) and thiosemicarbazide (1.56 g, 17.14 mmol, 1 equiv) in EtOH (30 mL) was added HOAc (3 mL) dropwise at room temperature. The resulting mixture was stirred for 4 h at 80° C. The reaction was quenched with Water at room temperature. The precipitated solids were collected by filtration and washed with water (2×15 mL). This resulted in (E)-[(5-bromofuran-2-yl)methylidene]aminothiourea (3.5 g, 82.28%) as a light yellow solid. LCMS (ES, m/z): [M+H]⁺: 248.

### Step 2. Synthesis of 5-(5-bromofuran-2-yl)-1,3,4-thiadiazol-2-amine

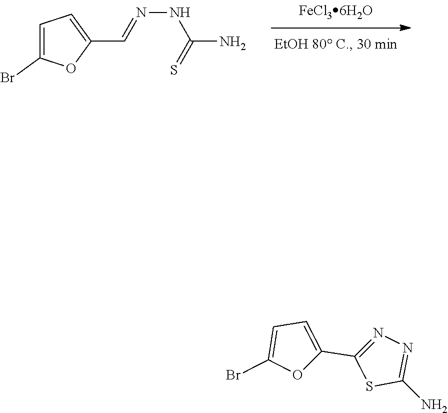 [SMILES: NC(=S)N/N=C/c1ccc(Br)o1 ; Nc1nnc(-c2ccc(Br)o2)s1]

To a stirred solution of (E)-[(5-bromofuran-2-yl)methylidene]aminothiourea (1 g, 4.03 mmol, 1 equiv) and ethyl alcohol (12 mL) was added ethyl alcohol (20 mL) in ethyl alcohol (8 mL) dropwise at room temperature. The resulting mixture was stirred for 30 min at 80° C. The resulting mixture was filtered, the filter cake was washed with ethanol (3×30 mL). The filtrate was concentrated under reduced pressure. The residue was purified by silica gel column chromatography, eluted with PE/THF (1:1) to afford 5-(5-bromofuran-2-yl)-1,3,4-thiadiazol-2-amine (160 mg, 16.13%) as a light-yellow oil. LCMS (ES, m/z): [M+H]⁺: 246.

### Step 3. Synthesis of tert-butyl (2S)-2-{[(1S)-2-{4-[5-(5-amino-1,3,4-thiadiazol-2-yl)furan-2-yl]phenyl}-1-cyanoethyl]carbamoyl}-1,4-oxazepane-4-carboxylate

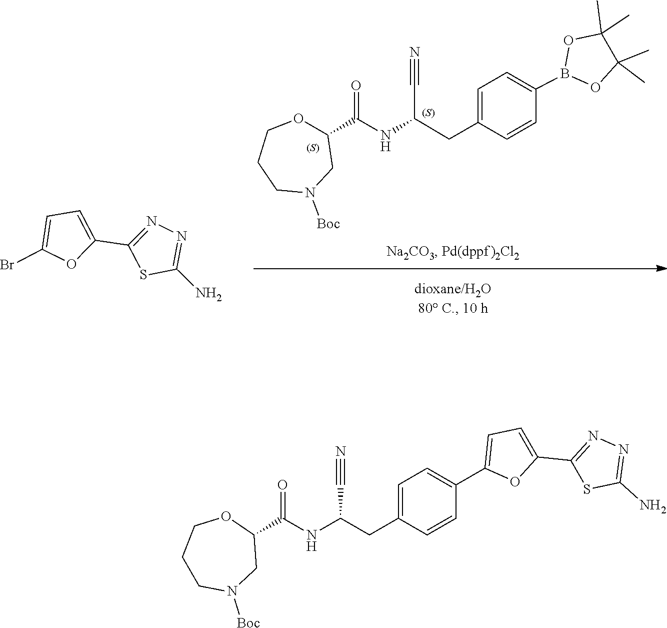

To a solution of 5-(5-bromofuran-2-yl)-1,3,4-thiadiazol-2-amine (100 mg, 0.40 mmol, 1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (202 mg, 0.40 mmol, 1 equiv) in dioxane (5 mL) and H₂O (0.5 mL) were added Na₂CO₃ (86 mg, 0.81 mmol, 2 equiv) and Pd(dppf)Cl₂CH₂Cl₂ (33 mg, 0.04 mmol, 0.1 equiv). After stirring for 3 h at 80° C. under a nitrogen atmosphere. To the above mixture was added Pd(dppf)Cl₂CH₂Cl₂ (33 mg, 0.04 mmol, 0.1 equiv) at room temperature. The resulting mixture was stirred for additional 3 h at 80° C. under a nitrogen atmosphere. the resulting mixture was concentrated under reduced pressure. The residue was purified by Prep-TLC/silica gel column chromatography, eluted with PE/THF (1:2) to afford tert-butyl (2S)-2-{[(1S)-2-{4-[5-(5-amino-1,3,4-thiadiazol-2-yl)furan-2-yl]phenyl}-1-cyanoethyl]carbamoyl}-1,4-oxazepane-4-carboxylate (80 mg, 36.55%) as a light yellow oil. LCMS (ES, m/z): [M+H]⁺: 539.

### Step 4. Synthesis of (2S)—N-[(1S)-2-{4-[5-(5-amino-1,3,4-thiadiazol-2-yl)furan-2-yl]phenyl}-1-cyanoethyl]-1,4-oxazepane-2-carboxamide

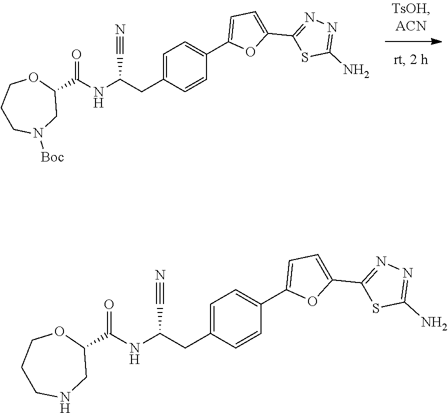 [SMILES: CC(C)(C)OC(=O)N1CCCO[C@H](C(=O)N[C@H](C#N)Cc2ccc(-c3ccc(-c4nnc(N)s4)o3)cc2)C1 ; N#C[C@H](Cc1ccc(-c2ccc(-c3nnc(N)s3)o2)cc1)NC(=O)[C@@H]1CNCCCO1]

Into a 50 mL round-bottom flask were added tert-butyl (2S)-2-{[(1S)-2-{4-[5-(5-amino-1,3,4-thiadiazol-2-yl)furan-2-yl]phenyl}-1-cyanoethyl]carbamoyl}-1,4-oxazepane-4-carboxylate (70 mg, 0.13 mmol, 1 equiv), TsOH·H₂O (74 mg, 0.39 mmol, 3 equiv) and ACN (3 mL) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in Compound D1 (16 mg, 28.08%) as a white solid. LCMS (ES, m/z): [M+H]⁺: 439. ¹H NMR (400 MHz, DMSO-d₆) δ 8.69 (d, J=8.4 Hz, 1H), 7.72 (d, J=8.0 Hz, 2H), 7.50 (s, 2H), 7.39 (d, J=8.1 Hz, 2H), 7.13 (d, J=3.6 Hz, 1H), 7.06 (d, J=3.6 Hz, 1H), 5.04 (q, J=8.5 Hz, 1H), 4.08-4.04 (m, 1H), 3.90-3.81 (m, 1H), 3.79-3.70 (m, 1H), 3.23-3.16 (m, 2H), 3.10 (d, J=13.9 Hz, 1H), 2.85 (s, 1H), 2.72-2.65 (m, 1H), 2.57-2.55 (m, 1H), 1.87-1.68 (m, 2H).

### Example 58: Synthesis of (2S)—N-[(1S)-2-{4-[5-(5-amino-1,3,4-oxadiazol-2-yl)thiophen-3-yl]phenyl}-1-cyanoethyl]-1,4-oxazepane-2-carboxamide (Compound D2)

### Step 1. Synthesis of (E)-[(4-bromothiophen-2-yl)methylidene]aminourea

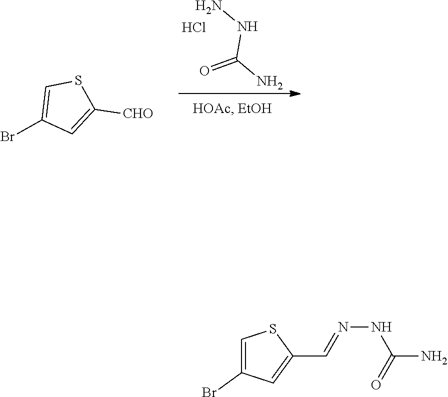 [SMILES: O=Cc1cc(Br)cs1 ; Cl ; NNC(N)=O ; NC(=O)N/N=C/c1cc(Br)cs1]

To a stirred solution of 4-bromothiophene-2-carbaldehyde (3 g, 15.704 mmol, 1 equiv) and semicarbazide hydrochloride (1.75 g, 15.70 mmol, 1 equiv) in EtOH (30 mL) was added HOAc (3 mL) dropwise at room temperature. The resulting mixture was stirred for 4 h at 80° C. The reaction was quenched with Water at room temperature. The precipitated solids were collected by filtration and washed with water (2×15 mL). This resulted in (E)-[(4-bromothiophen-2-yl)methylidene]aminourea (3.7 g, 94.97%) as a light yellow solid. LCMS (ES, m/z): [M+H]⁺: 248.

### Step 2. Synthesis of 5-(4-bromothiophen-2-yl)-1,3,4-oxadiazol-2-amine

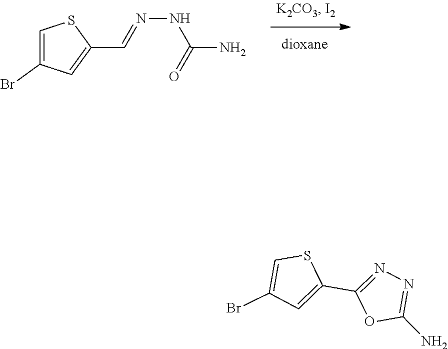 [SMILES: NC(=O)N/N=C/c1cc(Br)cs1 ; Nc1nnc(-c2cc(Br)cs2)o1]

To a stirred solution of (E)-[(4-bromothiophen-2-yl)methylidene]aminourea (1 g, 4.03 mmol, 1 equiv) and K₂CO₃ (1.67 g, 12.09 mmol, 3 equiv) in dioxane (20 mL) was added I₂ (1.23 g, 4.83 mmol, 1.2 equiv) at room temperature. The resulting mixture was stirred for 2 h at 100° C. The resulting mixture was filtered, the filter cake was washed with CH₂Cl₂ (3×20 mL). The filtrate was concentrated under reduced pressure. The residue was purified by silica gel column chromatography, eluted with PE/THF (1:1) to afford 5-(4-bromothiophen-2-yl)-1,3,4-oxadiazol-2-amine (350 mg, 35.29%) as a light yellow solid. LCMS (ES, m/z): [M+H]⁺: 246.

### Step 3. Synthesis of tert-butyl (2S)-2-{[(1S)-2-{4-[5-(5-amino-1,3,4-oxadiazol-2-yl)thiophen-3-yl]phenyl}-1-cyanoethyl]carbamoyl}-1,4-oxazepane-4-carboxylate

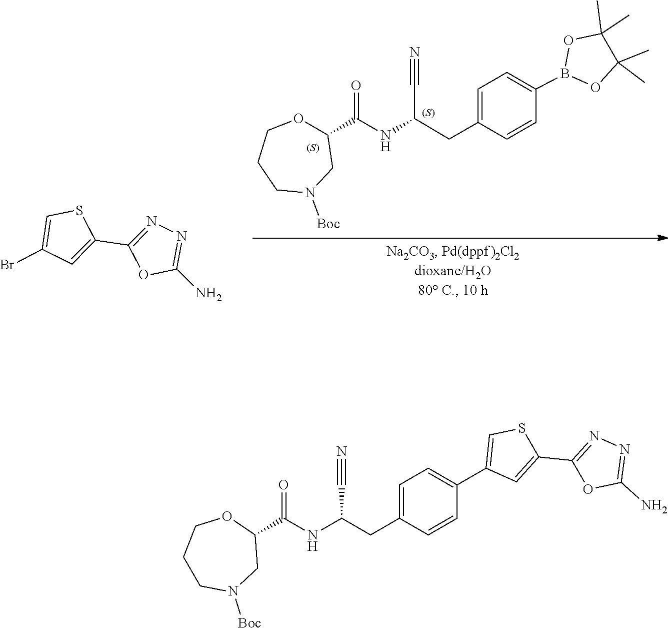

To a solution of 5-(4-bromothiophen-2-yl)-1,3,4-oxadiazol-2-amine (110 mg, 0.44 mmol, 1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (223 mg, 0.44 mmol, 1 equiv) in dioxane (5 mL) and H₂O (0.5 mL) were added Na₂CO₃ (94.76 mg, 0.894 mmol, 2 equiv) and Pd(dppf)Cl₂CH₂Cl₂ (36 mg, 0.04 mmol, 0.1 equiv). After stirring for 3 h at 80° C. under a nitrogen atmosphere. To the above mixture was added Pd(dppf)Cl₂CH₂Cl₂ (36 mg, 0.04 mmol, 0.1 equiv) at room temperature. The resulting mixture was stirred for additional 3 h at 80° C. under a nitrogen atmosphere. the resulting mixture was concentrated under reduced pressure. The residue was purified by silica gel column chromatography, eluted with PE/THF (1:2) to afford tert-butyl (2S)-2-{[(1S)-2-{4-[5-(5-amino-1,3,4-oxadiazol-2-yl)thiophen-3-yl]phenyl}-1-cyanoethyl]carbamoyl}-1,4-oxazepane-4-carboxylate (70 mg, 24.71%) as a light yellow oil. LCMS (ES, m/z): [M+H]⁺: 539.

### Step 4. Synthesis of (2S)—N-[(1S)-2-{4-[5-(5-amino-1,3,4-oxadiazol-2-yl)thiophen-3-yl]phenyl}-1-cyanoethyl]-1,4-oxazepane-2-carboxamide

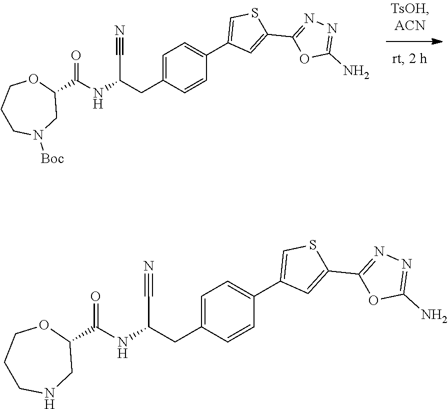 [SMILES: CC(C)(C)OC(=O)N1CCCO[C@H](C(=O)N[C@H](C#N)Cc2ccc(-c3csc(-c4nnc(N)o4)c3)cc2)C1 ; N#C[C@H](Cc1ccc(-c2csc(-c3nnc(N)o3)c2)cc1)NC(=O)[C@@H]1CNCCCO1]

Into a 50 mL round-bottom flask were added tert-butyl (2S)-2-{[(1S)-2-{4-[5-(5-amino-1,3,4-oxadiazol-2-yl)thiophen-3-yl]phenyl}-1-cyanoethyl]carbamoyl}-1,4-oxazepane-4-carboxylate (60 mg, 0.11 mmol, 1 equiv), TsOH·H₂O (63.57 mg, 0.33 mmol, 3 equiv) and ACN (3 mL) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in Compound D2 (15 mg, 30.71%) as a white solid. LCMS (ES, m/z): [M+H]⁺: 439. ¹H NMR (400 MHz, DMSO-d₆) δ 8.70 (d, J=8.5 Hz, 1H), 8.06 (d, J=1.5 Hz, 1H), 7.90 (d, J=1.5 Hz, 1H), 7.73 (d, J=8.3 Hz, 2H), 7.36 (d, J=8.2 Hz, 2H), 7.32 (s, 2H), 5.08-4.97 (m, 1H), 4.12-4.06 (m, 1H), 3.92-3.81 (m, 1H), 3.79-3.68 (m, 1H), 3.26-3.07 (m, 3H), 2.89-2.85 (m, 1H), 2.72-2.60 (m, 1H), 2.65-2.56 (m, 1H), 1.85-1.68 (m, 2H).

### Example 59: Synthesis of (2S)—N-[(1S)-1-cyano-2-(4-{1-[(3R)-oxolan-3-yl]pyrazol-4-yl}phenyl)ethyl]-1,4-oxazepane-2-carboxamide (Compound D4)

### Step 1. Synthesis of (S)-tetrahydrofuran-3-yl 4-methylbenzenesulfonate

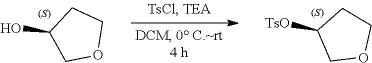

A solution of (3s)-tetrahydrofuran-3-ol (2.0 g, 22.7 mmol, 1.0 equiv), TEA (6.9 g, 68.2 mmol, 3.0 equiv) were stirred in DCM (50 mL) and cooled to 0° C., this was followed by the addition of 4-Methylbenzenesulfonyl chloride (5.2 g, 27.2 mmol, 1.2 equiv). The mixture stirred at rt for 4 h. TLC (50% EtOAc in hexane) indicated complete consumption of alcohol. The reaction mixture was washed with 1 M HCl (2×50 mL) and the organic phase was dried over anhydrous sodium sulfate. Filtered and the filtrate was concentrated under reduced pressure to give the title compound 4.8 g as yellow viscous oil. ¹H NMR (400 MHz, CDCl₃): δ 7.79 (d, J=8.3 Hz, 2H), 7.35 (d, J=8.0 Hz, 2H), 5.11 (tt, J=4.7, 2.3 Hz, 1H), 3.91-3.78 (m, 4H), 2.45 (s, 3H), 2.12-2.07 (m, 2H).

### Step 2. Synthesis of (R)-4-iodo-1-(tetrahydrofuran-3-yl)-1H-pyrazole

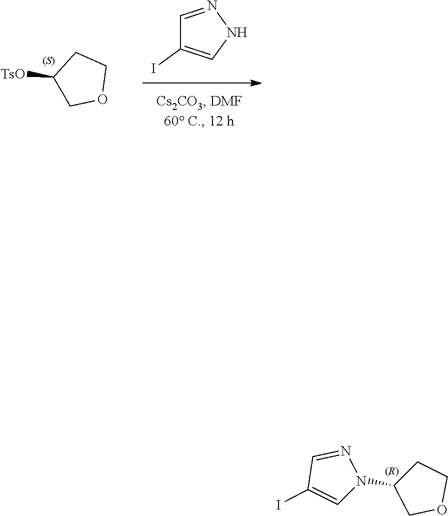

To a solution of 4-iodo-1H-pyrazole (1.0 g, 5.2 mmol, 1.0 equiv) in DMF (20 mL) were added Cs₂CO₃ (2.5 g, 7.8 mmol, 1.5 equiv) and (S)-tetrahydrofuran-3-yl 4-methylbenzenesulfonate (1.4 g, 5.7 mmol, 1.1 equiv) in sequence at room temperature. The mixture was heated to 60° C. and stirred for 12 h. The reaction was cooled to room temperature, diluted with water (30 mL), extracted with ethyl acetate (40 mL×2). The combined organic phase was washed with brine (20 mL×3), dried over anhydrous sodium sulfate. Filtered and the filtrate was concentrated under reduced pressure. The residue was purified by silica gel column chromatography, eluted with petroleum ether/ethyl acetate (5:1) to afford 1.0 g (R)-4-iodo-1-(tetrahydrofuran-3-yl)-1H-pyrazole as off-white solid. LCMS (ES, m/z): [M+H]⁺: 265.

### Step 3. Synthesis of tert-butyl (2S)-2-{[(1S)-1-cyano-2-(4-{1-[(3R)-oxolan-3-yl]pyrazol-4-yl}phenyl)ethyl]carbamoyl}-1,4-oxazepane-4-carboxylate

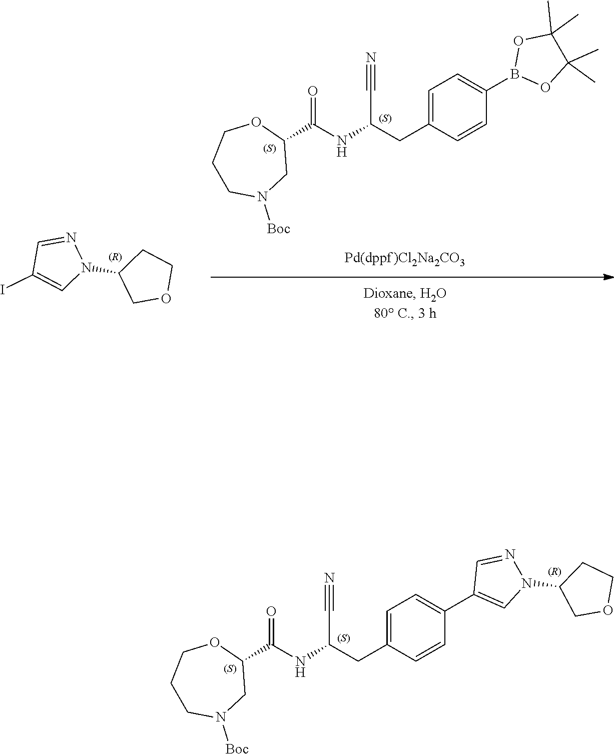

To a solution of 4-iodo-1-[(3R)-oxolan-3-yl]pyrazole (130 mg, 0.49 mmol, 1.0 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (295 mg, 0.59 mmol, 1.2 equiv) in dioxane (3 mL) and H₂O (0.3 mL) were added Na₂CO₃ (104 mg, 0.98 mmol, 2.0 equiv) and Pd(dppf)Cl₂ (40 mg, 0.05 mmol, 0.1 equiv) in sequence. The mixture was stirred for 3 h at 80° C. under nitrogen atmosphere. The reaction was cooled to room temperature, concentrated under reduced pressure. The residue was purified by silica gel column chromatography, eluted with PE/THF (4:1) to afford tert-butyl (2S)-2-{[(1S)-1-cyano-2-(4-{1-[(3R)-oxolan-3-yl]pyrazol-4-yl}phenyl)ethyl]carbamoyl}-1,4-oxazepane-4-carboxylate (85 mg, 34%) as yellow semi-solid. LCMS (ES, m/z): [M+H]⁺: 510.

### Step 4. Synthesis of (2S)—N-[(1S)-1-cyano-2-(4-{1-[(3R)-oxolan-3-yl]pyrazol-4-yl}phenyl)ethyl]-1,4-oxazepane-2-carboxamide

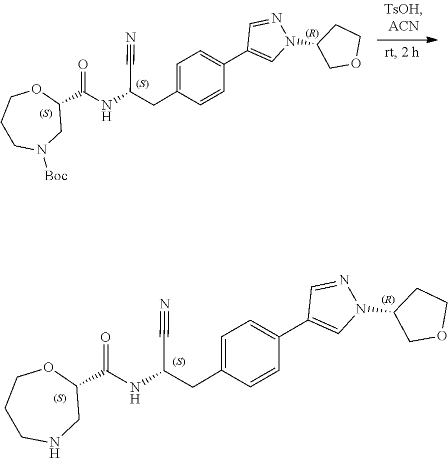

Into a 8 mL vial were added tert-butyl (2S)-2-{[(1S)-1-cyano-2-(4-{1-[(3R)-oxolan-3-yl]pyrazol-4-yl}phenyl)ethyl]carbamoyl}-1,4-oxazepane-4-carboxylate (80 mg, 0.15 mmol, 1.0 equiv), TsOH (81 mg, 0.47 mmol, 3.0 equiv) and ACN (1 mL) at room temperature. The resulting mixture was stirred for 2 h at room temperature. The reaction solution was purified by reversed-phase flash chromatography with the following conditions: column, C18 silica gel-120 g; mobile phase, MeCN in Water (0.1% NH₃·H₂O), 10% to 80% gradient in 10 min; detector, UV 254 nm. This resulted in Compound D4 (25 mg, 38%) as off-white solid. LCMS (ES, m/z): [M+H]⁺: 410.1. ¹H NMR (400 MHz, DMSO-d₆) δ 8.58 (d, J=8.5 Hz, 1H), 8.22 (s, 1H), 7.90 (s, 1H), 7.54 (d, J=7.9 Hz, 2H), 7.27 (d, J=7.9 Hz, 2H), 5.11-4.86 (m, 2H), 4.04-3.95 (m, 3H), 3.92 (dd, J=9.4, 3.8 Hz, 1H), 3.89-3.80 (m, 2H), 3.72 (ddd, J=12.2, 7.7, 4.0 Hz, 1H), 3.21-3.08 (m, 2H), 3.03 (dd, J=14.3, 3.7 Hz, 1H), 2.77 (dt, J=13.2, 5.5 Hz, 1H), 2.69-2.52 (m, 2H), 2.47-2.25 (m, 2H), 1.82-1.65 (m, 2H).

### Example 60: Synthesis of (2S)—N-[(1S)-1-cyano-2-{4-[2-(1,3-thiazol-4-yl)-1,3-thiazole-4-amido]phenyl}ethyl]-1,4-oxazepane-2-carboxamide (Compound D7)

### Step 1. Synthesis of 1,3-thiazole-4-carboxamide

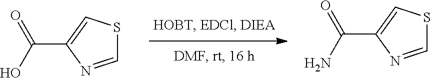 [SMILES: O=C(O)c1cscn1 ; NC(=O)c1cscn1]

To a stirred solution of 1,3-thiazole-4-carboxylic acid (5 g, 38.72 mmol, 1.0 equiv) and NH₄Cl (6.21 g, 116.16 mmol, 3.0 equiv) in DMF (20 mL) was added DIEA (15.0 g, 116.16 mmol, 3.0 equiv), HOBT (6.28 g, 46.46 mmol, 1.2 equiv), EDCI (8.91 g, 46.46 mmol, 1.2 equiv) at room temperature. The resulting mixture was stirred for 16 h at room temperature. The reaction was quenched with water (20 mL), extracted with EtOAc (3×100 mL). The combined organic layer was washed with brine (3×50 mL), dried over anhydrous Na₂SO₄. After filtration, the filtrate was concentrated under reduced pressure. The residue was purified by silica gel column chromatography, eluted with PE/EA (1:1) to afford 1,3-thiazole-4-carboxamide (3 g, 60.4%) as a light yellow oil. LCMS (ES, m/z): [M+H]⁺: 129.

### Step 2. Synthesis of 1,3-thiazole-4-carbothioamide

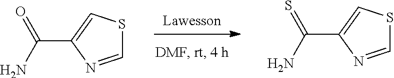 [SMILES: NC(=O)c1cscn1 ; NC(=S)c1cscn1]

To a stirred solution of 1,3-thiazole-4-carboxamide (3 g, 23.41 mmol, 1.0 equiv) in DMF (40 mL) was added Lawesson Reagent (4.73 g, 11.70 mmol, 0.5 equiv) at room temperature. The resulting mixture was stirred for 4 h at room temperature. The reaction was quenched with water (50 mL), extracted with EtOAc (3×100 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (2:1) to afford 1,3-thiazole-4-carbothioamide (2.5 g, 74.0%) as a light yellow oil. LCMS (ES, m/z): [M+H]⁺: 145.

### Step 3. Synthesis of ethyl 2-(1,3-thiazol-4-yl)-1,3-thiazole-4-carboxylate

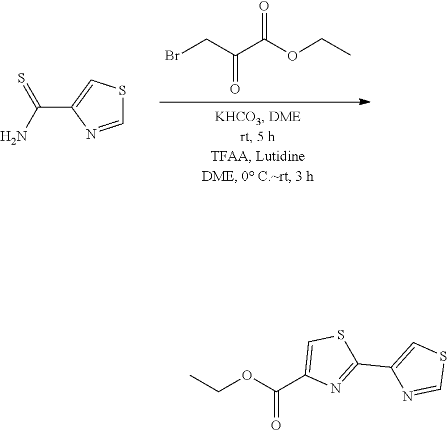

To a stirred solution of 1,3-thiazole-4-carbothioamide (2.5 g, 17.33 mmol, 1.0 equiv) in DME (30 mL) was added KHCO₃ (8.7 g, 86.68 mmol, 5.0 equiv) at room temperature. The resulting mixture was stirred for 10 min at room temperature. To the above mixture was added ethyl 3-bromo-2-oxopropanoate (5.07 g, 26.00 mmol, 1.5 equiv) at room temperature. The resulting mixture was stirred for additional 30 min at room temperature. To the above mixture was added TFAA (18.21 g, 86.68 mmol, 5.0 equiv) and 2,6-lutidine (5.57 g, 52.00 mmol, 3.0 equiv) in DME (20 mL) at 0° C. The resulting mixture was stirred for additional 40 min at the same temperature. The resulting mixture was stirred for 12 h at room temperature, diluted with water (100 mL), was extracted with EtOAc (3×100 mL). The combined organic layer was dried over anhydrous Na₂SO₄. After filtration, the filtrate was concentrated under reduced pressure. The residue was purified by silica gel column chromatography, eluted with PE/EA (2:1) to afford ethyl 2-(1,3-thiazol-4-yl)-1,3-thiazole-4-carboxylate (2 g, 48.01%) as a light yellow solid. LCMS (ES, m/z): [M+H]⁺: 241.

### Step 4. Synthesis of 2-(1,3-thiazol-4-yl)-1,3-thiazole-4-carboxylic acid

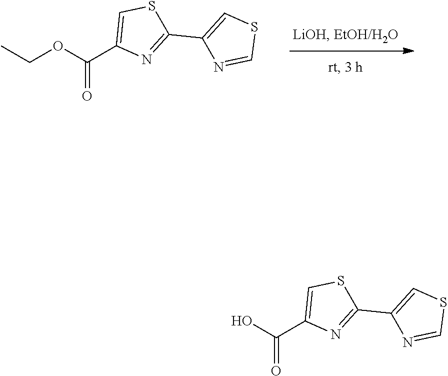

To a stirred solution of ethyl 2-(1,3-thiazol-4-yl)-1,3-thiazole-4-carboxylate (1 g, 4.16 mmol, 1.0 equiv) in EtOH (9 mL) was added LiOH·H₂O (0.35 g, 8.32 mmol, 2.0 equiv) in H₂O (3 mL) dropwise at room temperature. The resulting mixture was stirred for 3 h at room temperature. The resulting mixture was concentrated under reduced pressure. The mixture was acidified to pH 3 with conc. HCl. The precipitated solid was collected by filtration and washed with water (3×10 mL). The solid was dried under infrared lamp for 4 h. This resulted in 2-(1,3-thiazol-4-yl)-1,3-thiazole-4-carboxylic acid (800 mg, 90.6%) as a light yellow solid. LCMS (ES, m/z): [M+H]⁺: 213.

### Step 5. Synthesis of tert-butyl (2S)-2-{[(1S)-1-cyano-2-{4-[(diphenylmethylidene)amino]phenyl}ethyl]carbamoyl}-1,4-oxazepane-4-carboxylate

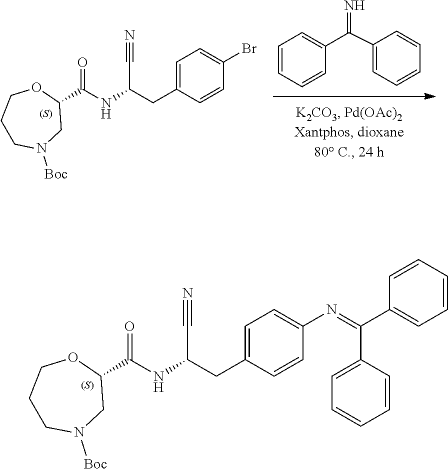

To a solution of tert-butyl (2S)-2-{[(1S)-2-(4-bromophenyl)-1-cyanoethyl]carbamoyl}-1,4-oxazepane-4-carboxylate (1 g, 2.21 mmol, 1.0 equiv) and diphenylmethanimine (0.48 g, 2.65 mmol, 1.2 equiv) in dioxane (15 mL) were added K₂CO₃ (0.92 g, 6.63 mmol, 3.0 equiv), Pd(OAc)₂ (0.05 g, 0.22 mmol, 0.1 equiv) and XantPhos (0.26 g, 0.44 mmol, 0.2 equiv). After stirring for 24 h at 80° C. under nitrogen atmosphere, the resulting mixture was concentrated under reduced pressure. The residue was purified by silica gel column chromatography, eluted with PE/EA (1:1) to afford tert-butyl (2S)-2-{[(1S)-1-cyano-2-{4-[(diphenylmethylidene)amino]phenyl}ethyl]carbamoyl}-1,4-oxazepane-4-carboxylate (500 mg, 40.9%) as a yellow oil. LCMS (ES, m/z): [M+H]⁺: 553.

### Step 6. Synthesis of tert-butyl (2S)-2-{[(1S)-2-(4-aminophenyl)-1-cyanoethyl]carbamoyl}-1,4-oxazepane-4-carboxylate

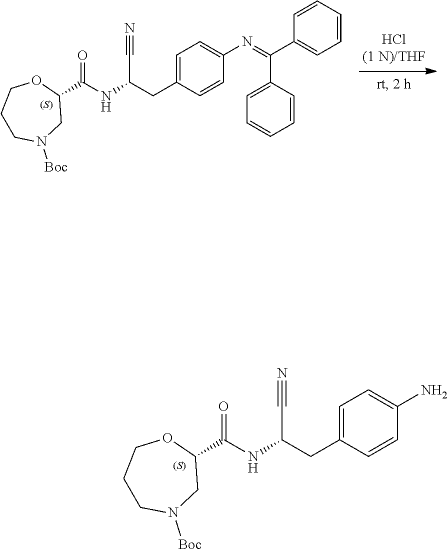

To a stirred solution of tert-butyl (2S)-2-{[(1S)-1-cyano-2-{4-[(diphenylmethylidene)amino]phenyl}ethyl]carbamoyl}-1,4-oxazepane-4-carboxylate (500 mg, 0.90 mmol, 1.0 equiv) in THF (20 mL) and H₂O (2 mL) was added HCl (1M) (1 mL) at room temperature. The resulting mixture was stirred for 2 h at room temperature. The mixture was acidified to pH 10 with saturated K₂CO₃ (aq.). The resulting mixture was extracted with EtOAc (3×30 mL). The combined organic layer was dried over anhydrous Na₂SO₄. After filtration, the filtrate was concentrated under reduced pressure. The residue was purified by silica gel column chromatography, eluted with PE/THF (1:2) to afford tert-butyl (2S)-2-{[(1S)-2-(4-aminophenyl)-1-cyanoethyl]carbamoyl}-1,4-oxazepane-4-carboxylate (200 mg, 56.9%) as a light yellow oil. LCMS (ES, m/z): [M+H]⁺: 389.

### Step 7. Synthesis of tert-butyl (2S)-2-{[(1S)-1-cyano-2-{4-[2-(1,3-thiazol-4-yl)-1,3-thiazole-4-amido]phenyl}ethyl]carbamoyl}-1,4-oxazepane-4-carboxylate

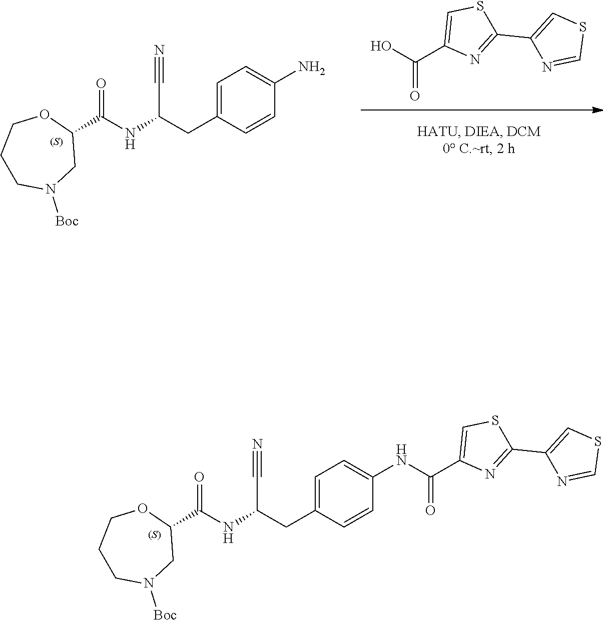

To a stirred mixture of tert-butyl (2S)-2-{[(1S)-2-(4-aminophenyl)-1-cyanoethyl]carbamoyl}-1,4-oxazepane-4-carboxylate (110 mg, 0.28 mmol, 1.0 equiv), 2-(1,3-thiazol-4-yl)-1,3-thiazole-4-carboxylic acid (60 mg, 0.28 mmol, 1.0 equiv) and DIEA (109 mg, 0.84 mmol, 3.0 equiv) in DCM (5 mL) was added HATU (129 mg, 0.34 mmol, 1.2 equiv) in portions at 0° C. The resulting mixture was stirred for additional 2 h at 0° C. Concentrated to remove the solvent, the residue was purified by silica gel column chromatography, eluted with PE/THF (1:1) to afford tert-butyl (2S)-2-{[(1S)-1-cyano-2-{4-[2-(1,3-thiazol-4-yl)-1,3-thiazole-4-amido]phenyl}ethyl]carbamoyl}-1,4-oxazepane-4-carboxylate (140 mg, 84.8%) as a light yellow solid. LCMS (ES, m/z): [M+H]⁺: 583.

### Step 8. Synthesis of (2S)—N-[(1S)-1-cyano-2-{4-[2-(1,3-thiazol-4-yl)-1,3-thiazole-4-amido]phenyl}ethyl]-1,4-oxazepane-2-carboxamide

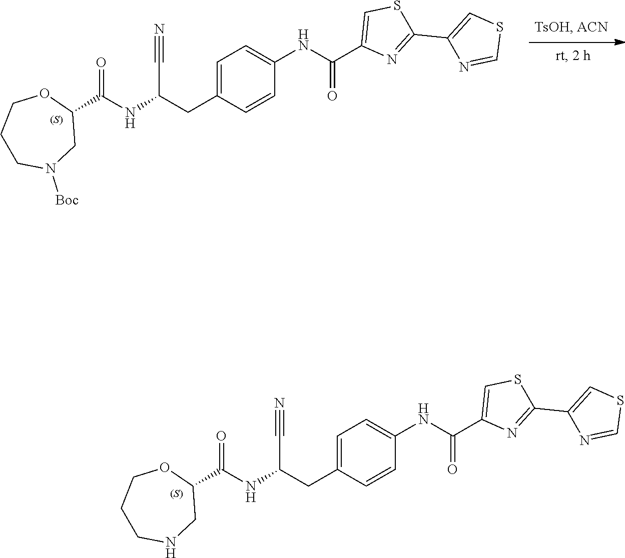

Into a 25 mL round-bottom flask were added tert-butyl (2S)-2-{[(1S)-1-cyano-2-{4-[2-(1,3-thiazol-4-yl)-1,3-thiazole-4-amido]phenyl}ethyl]carbamoyl}-1,4-oxazepane-4-carboxylate (130 mg, 0.22 mmol, 1.0 equiv), TsOH·H₂O (127 mg, 0.66 mmol, 3.0 equiv) and ACN (4 mL) at room temperature. The resulting mixture was stirred for 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound D7 (20 mg, 18.6%) as a white solid. LCMS (ES, m/z): [M+H]⁺: 483.3. ¹H NMR (400 MHz, DMSO-d₆): δ 10.21 (s, 1H), 9.30 (d, J=2.0 Hz, 1H), 8.60 (d, J=8.5 Hz, 1H), 8.52 (d, J=2.0 Hz, 1H), 8.47 (s, 1H), 7.83-7.75 (m, 2H), 7.33-7.27 (m, 2H), 5.02-4.95 (m, 1H), 4.00 (dd, J=7.9, 3.7 Hz, 1H), 3.90-3.83 (m, 1H), 3.77-3.70 (m, 1H), 3.22-3.10 (m, 2H), 3.05 (dd, J=14.3, 3.7 Hz, 1H), 2.81-2.74 (m, 1H), 2.69-2.53 (m, 2H), 2.31 (br, 1H), 1.83-1.67 (m, 2H).

### Example 61: Synthesis of (2S)—N-[(1S)-1-cyano-2-{4-[5-ethyl-4-(1-methylpyrazol-4-yl)-1,3-thiazol-2-yl]phenyl}ethyl]-1,4-oxazepane-2-carboxamide (Compound D8)

### Step 1. Synthesis of 2-bromo-5-ethyl-1,3-thiazole

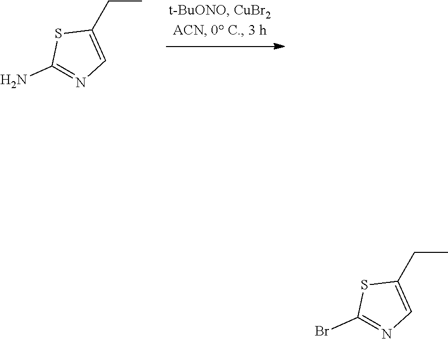

To a stirred solution of 5-ethyl-1,3-thiazol-2-amine (2.0 g, 15.60 mmol, 1.0 equiv) in ACN (30 mL) were added t-BuONO (1.8 g, 17.16 mmol, 1.1 equiv) and CuBr₂ (3.8 g, 17.16 mmol, 1.1 equiv) in portions at 0° C., under nitrogen atmosphere. The resulting mixture was stirred for 3 h at 0° C. The resulting mixture was diluted with water (50 mL). The resulting mixture was extracted with EtOAc (3×30 mL). The combined organic layer was washed with brine (30 mL), dried over anhydrous Na₂SO₄. After filtration, the filtrate was concentrated under reduced pressure. The residue was purified by silica gel column chromatography, eluted with PE/EA (20:1) to afford 2-bromo-5-ethyl-1,3-thiazole (1.8 g, 60.1%) as colorless oil. LCMS (ES, m/z): [M+H]⁺: 192.

### Step 2. Synthesis of 2,4-dibromo-5-ethyl-1,3-thiazole

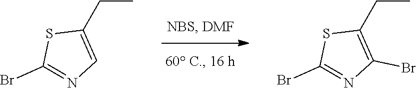

To a stirred solution of 2-bromo-5-ethyl-1,3-thiazole (1.6 g, 8.33 mmol, 1.0 equiv) in DMF (30 mL) was added NBS (2.9 g, 16.68 mmol, 2.0 equiv). The resulting mixture was stirred for 16 h at 60° C. under nitrogen atmosphere. The mixture was allowed to cool down to room temperature. The reaction was diluted with water (30 mL), extracted with ethyl acetate (30 mL×2). The combined organic phase was washed with brine (30 mL×2), dried over anhydrous sodium sulfate. After filtration, the filtrate was concentrated under reduced pressure. The crude product was purified by Prep-HPLC with the following conditions: Column, XBridge Prep C18 OBD Column, 19*150 mm, 5 um; mobile phase, Water (0.1% NH₃H₂O) and ACN (10% Phase B up to 80% in 20 min); Detector, UV 254 nm. This resulted in 2,4-dibromo-5-ethyl-1,3-thiazole (0.9 g, 39.8%) as light-yellow oil. LCMS (ES, m/z): [M+H]⁺: 270.

### Step 3. Synthesis of tert-butyl (2S)-2-{[(1S)-2-[4-(4-bromo-5-ethyl-1,3-thiazol-2-yl)phenyl]-1-cyanoethyl]carbamoyl}-1,4-oxazepane-4-carboxylate

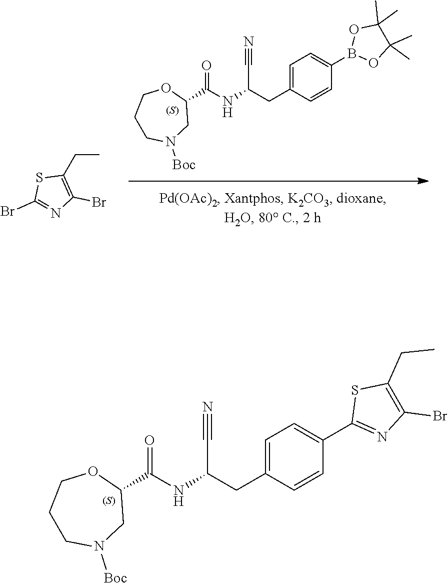

To a stirred solution of 2,4-dibromo-5-ethyl-1,3-thiazole (200 mg, 0.73 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (0.4 g, 0.73 mmol, 1.0 equiv) in dioxane (3 mL) and H₂O (0.3 mL) were added K₂CO₃ (204 mg, 1.47 mmol, 2.0 equiv) and Xantphos (85 mg, 0.14 mmol, 0.2 equiv) and Pd(OAc)₂ (17 mg, 0.07 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (4:1) to afford tert-butyl (2S)-2-{[(1S)-2-[4-(4-bromo-5-ethyl-1,3-thiazol-2-yl)phenyl]-1-cyanoethyl]carbamoyl}-1,4-oxazepane-4-carboxylate (200 mg, 48.0%) as off-white solid. LCMS (ES, m/z): [M+H]⁺: 563.

### Step 4. Synthesis of tert-butyl (2S)-2-{[(1S)-1-cyano-2-{4-[5-ethyl-4-(1-methylpyrazol-4-yl)-1,3-thiazol-2-yl]phenyl}ethyl]carbamoyl}-1,4-oxazepane-4-carboxylate

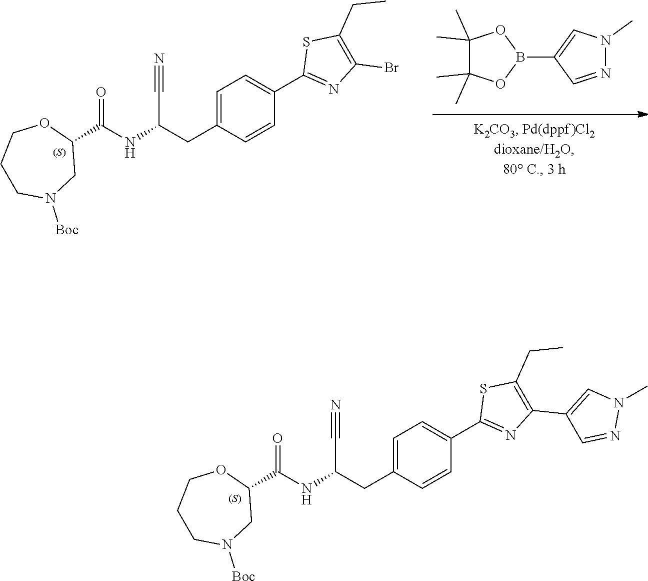

To a solution of tert-butyl (2S)-2-{[(1S)-2-[4-(4-bromo-5-ethyl-1,3-thiazol-2-yl)phenyl]-1-cyanoethyl]carbamoyl}-1,4-oxazepane-4-carboxylate (270 mg, 0.47 mmol, 1.0 equiv) and 1-methyl-4-(4,4,5,5-tetramethyl-1,3,2-dioxaborolan-2-yl)pyrazole (149 mg, 0.71 mmol, 1.5 equiv) in 1,4-dioxane (5 mL) H₂O (0.5 mL) were added K₂CO₃ (132 mg, 0.95 mmol, 2.0 equiv), Pd(dppf)Cl₂ (35 mg, 0.04 mmol, 0.1 equiv). The mixture was stirred for 2 h at 80° C. under nitrogen atmosphere. The reaction was cooled to room temperature, concentrated to remove the solvent. The residue was purified by silica gel column chromatography, eluted with PE/THF (1:1) to afford tert-butyl (2S)-2-{[(1S)-1-cyano-2-{4-[5-ethyl-4-(1-methylpyrazol-4-yl)-1,3-thiazol-2-yl]phenyl}ethyl]carbamoyl}-1,4-oxazepane-4-carboxylate (200 mg, 73.9%) as white semi-solid. LCMS (ES, m/z): [M+H]⁺: 565.

### Step 5. Synthesis of (2S)—N-[(1S)-1-cyano-2-{4-[5-ethyl-4-(1-methylpyrazol-4-yl)-1,3-thiazol-2-yl]phenyl}ethyl]-1,4-oxazepane-2-carboxamide

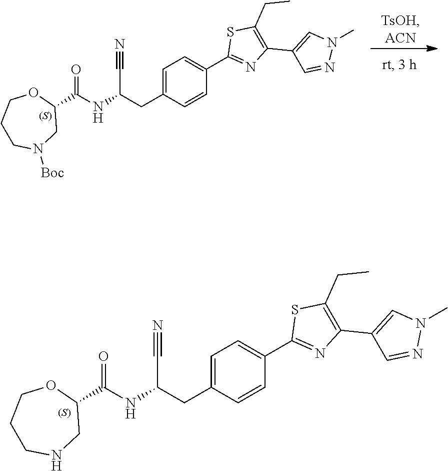

Into a 25 mL round-bottom flask were added tert-butyl (2S)-2-{[(1S)-1-cyano-2-{4-[5-ethyl-4-(1-methylpyrazol-4-yl)-1,3-thiazol-2-yl]phenyl}ethyl]carbamoyl}-1,4-oxazepane-4-carboxylate (100 mg, 0.17 mmol, 1.0 equiv) in ACN (3 mL) and TsOH·H₂O (91 mg, 0.53 mmol, 3.0 equiv) at room temperature. The resulting mixture was stirred for additional 3 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound D8 (17.3 mg, 21%) as white solid. LCMS (ES, m/z): [M+H]⁺: 465.4. ¹H NMR (400 MHz, DMSO-d₆) δ 8.63 (d, J=8.6 Hz, 1H), 8.12 (s, 1H), 7.90-7.84 (m, 2H), 7.82 (d, J=0.8 Hz, 1H), 7.42-7.40 (m, 2H), 5.11-5.00 (m, 1H), 3.99 (dd, J=7.9, 3.7 Hz, 1H), 3.91 (s, 3H), 3.90-3.80 (m, 1H), 3.76-3.69 (m, 1H), 3.25-3.18 (m, 2H), 3.05-2.93 (m, 3H), 2.80-2.73 (m, 1H), 2.64-2.57 (m, 1H), 2.55-2.52 (m, 1H), 1.79-1.69 (m, 2H), 1.31 (t, J=7.5 Hz, 3H).

### Example 62: Synthesis of (2S)—N-[(1S)-1-cyano-2-{4-[5-methyl-4-(2-oxo-1H-pyridin-4-yl)-1,3-thiazol-2-yl]phenyl}ethyl]-1,4-oxazepane-2-carboxamide (Compound

D9)

### Step 1. Synthesis of tert-butyl (2S)-2-{[(1S)-2-[4-(4-bromo-5-methyl-1,3-thiazol-2-yl)phenyl]-1-cyanoethyl]carbamoyl}-1,4-oxazepane-4-carboxylate

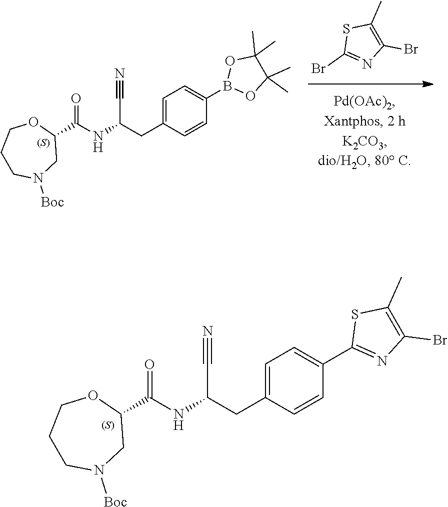

To a solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (454 mg, 0.91 mmol, 1.3 equiv) and 2,4-dibromo-5-methyl-1,3-thiazole (180 mg, 0.70 mmol, 1.0 equiv) in dioxane (10 mL) and H₂O (1 mL) were added K₂CO₃ (290 mg, 2.10 mmol, 3.0 equiv) and Pd(OAc)₂ (15 mg, 0.07 mmol, 0.1 equiv), XantPhos (81 mg, 0.14 mmol, 0.2 equiv) in sequence. After stirring for 2 h at 80° C. under nitrogen atmosphere, the resulting mixture was concentrated under reduced pressure. The residue was purified by silica gel column chromatography, eluted with PE/EA (1:1) to afford tert-butyl (2S)-2-{[(1S)-2-[4-(4-bromo-5-methyl-1,3-thiazol-2-yl)phenyl]-1-cyanoethyl]carbamoyl}-1,4-oxazepane-4-carboxylate (340 mg, 88.3%) as a light yellow oil. LCMS (ES, m/z): [M+H]⁺: 549.

### Step 2. Synthesis of tert-butyl (2S)-2-{[(1S)-1-cyano-2-{4-[5-methyl-4-(2-oxo-1H-pyridin-4-yl)-1,3-thiazol-2-yl]phenyl}ethyl]carbamoyl}-1,4-oxazepane-4-carboxylate

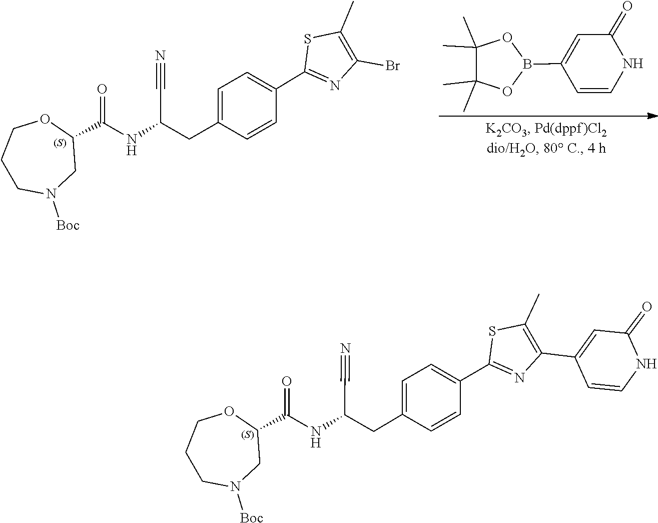

To a solution of tert-butyl (2S)-2-{[(1S)-2-[4-(4-bromo-5-methyl-1,3-thiazol-2-yl)phenyl]-1-cyanoethyl]carbamoyl}-1,4-oxazepane-4-carboxylate (340 mg, 0.61 mmol, 1.0 equiv) and 4-(4,4,5,5-tetramethyl-1,3,2-dioxaborolan-2-yl)-1H-pyridin-2-one (150 mg, 0.68 mmol, 1.1 equiv) in dioxane (10 mL) and H₂O (1 mL) were added K₂CO₃ (256 mg, 1.85 mmol, 3.0 equiv) and Pd(dppf)Cl₂CH₂Cl₂ (50 mg, 0.06 mmol, 0.1 equiv). After stirring for 4 h at 80° C. under nitrogen atmosphere, the resulting mixture was concentrated under reduced pressure. The residue was purified by silica gel column chromatography, eluted with PE/EA (1:1) to afford tert-butyl (2S)-2-{[(1S)-1-cyano-2-{4-[5-methyl-4-(2-oxo-1H-pyridin-4-yl)-1,3-thiazol-2-yl]phenyl}ethyl]carbamoyl}-1,4-oxazepane-4-carboxylate (250 mg, 71.7%) as a light yellow oil. LCMS (ES, m/z): [M+H]⁺: 564.

### Step 2. Synthesis of (2S)—N-[(1S)-1-cyano-2-{4-[5-methyl-4-(2-oxo-1H-pyridin-4-yl)-1,3-thiazol-2-yl]phenyl}ethyl]-1,4-oxazepane-2-carboxamide

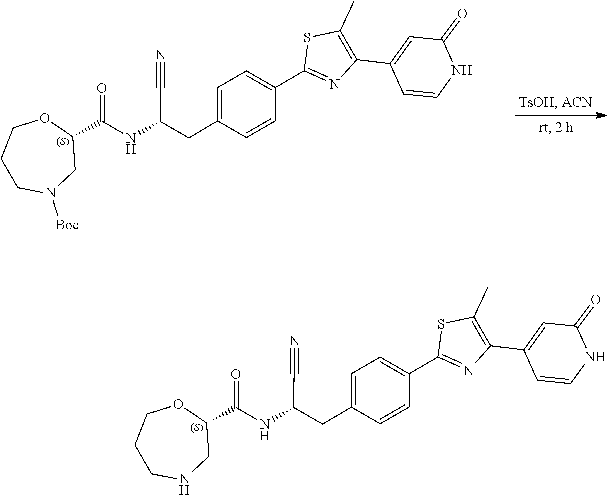

Into a 25 mL round-bottom flask were added tert-butyl (2S)-2-{[(1S)-1-cyano-2-{4-[5-methyl-4-(2-oxo-1H-pyridin-4-yl)-1,3-thiazol-2-yl]phenyl}ethyl]carbamoyl}-1,4-oxazepane-4-carboxylate (230 mg, 0.40 mmol, 1.0 equiv), TsOH·H₂O (215 mg, 1.13 mmol, 3.0 equiv) and ACN (4 mL) at room temperature. The resulting mixture was stirred for 2 h at room temperature. The reaction solution was purified by reversed-phase flash chromatography with the following conditions: column, C18 silica gel-120 g; mobile phase, MeCN in Water (0.1% NH₃·H₂O), 10% to 50% gradient in 10 min; detector, UV 254 nm. This resulted in Compound D9 (30 mg, 15.9%) as a white solid. LCMS (ES, m/z): [M+H]⁺: 464.3. ¹H NMR (400 MHz, DMSO-d₆): δ 11.60 (br, 1H), 8.63 (d, J=8.6 Hz, 1H), 7.87 (d, J=7.9 Hz, 2H), 7.47-7.41 (m, 3H), 6.62-6.59 (m, 2H), 5.06 (q, J=8.2 Hz, 1H), 3.99 (dd, J=7.9, 3.7 Hz, 1H), 3.88-3.82 (m, 1H), 3.75-3.69 (m, 1H), 3.24-3.18 (m, 2H), 3.01 (dd, J=14.3, 3.7 Hz, 1H), 2.80-2.73 (m, 1H), 2.64-2.56 (m, 4H), 2.49-2.47 (m, 1H), 1.86-1.63 (m, 2H).

### Example 63: Synthesis of (2S)—N-[(1S)-2-{4-[3-(2-butoxyethyl)-2-oxo-1,3-benzoxazol-5-yl]-2-fluorophenyl}-1-cyanoethyl]-6-methoxy-5-methyl-1,4-oxazepane-2-carboxamide (Compound E1), (2S,5S,6S)—N-[(1S)-2-{4-[3-(2-butoxyethyl)-2-oxo-1,3-benzoxazol-5-yl]-2-fluorophenyl}-1-cyanoethyl]-6-methoxy-5-methyl-1,4-oxazepane-2-carboxamide (Compound E2), (2S,5R,6R)—N-[(1S)-2-{4-[3-(2-butoxyethyl)-2-oxo-1,3-benzoxazol-5-yl]-2-fluorophenyl}-1-cyanoethyl]-6-methoxy-5-methyl-1,4-oxazepane-2-carboxamide (Compound E3), and (2S,5S,6R)—N-[(1S)-2-{4-[3-(2-butoxyethyl)-2-oxo-1,3-benzoxazol-5-yl]-2-fluorophenyl}-1-cyanoethyl]-6-methoxy-5-methyl-1,4-oxazepane-2-carboxamide (Compound E4)

### Step 1. Synthesis of tert-butyl (2S)-2-{[(1S)-2-{4-[3-(2-butoxyethyl)-2-oxo-1,3-benzoxazol-5-yl]-2-fluorophenyl}-1-cyanoethyl]carbamoyl}-6-methoxy-5-methyl-1,4-oxazepane-4-carboxylate

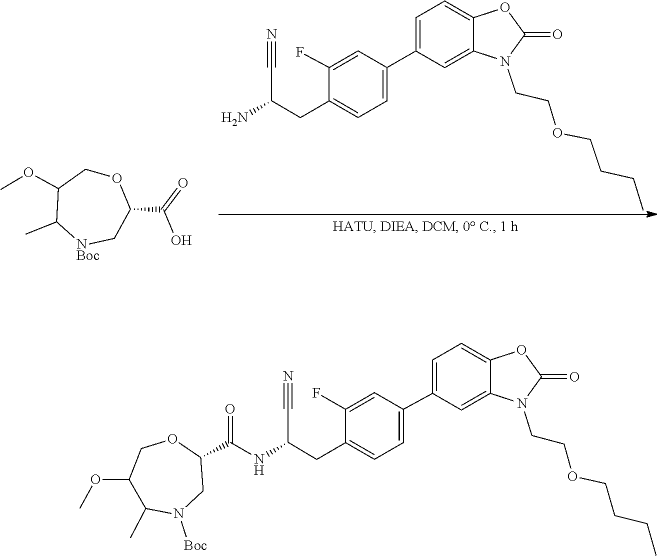

To a stirred solution of (2S)-4-(tert-butoxycarbonyl)-6-methoxy-5-methyl-1,4-oxazepane-2-carboxylic acid (100 mg, 0.346 mmol, 1.0 equiv) and (2S)-2-amino-3-{4-[3-(2-butoxyethyl)-2-oxo-1,3-benzoxazol-5-yl]-2-fluorophenyl}propanenitrile (137 mg, 0.346 mmol, 1.0 equiv) in DCM (3 mL) were added DIEA (134 mg, 1.038 mmol, 3.0 equiv) and HATU (197 mg, 0.519 mmol, 1.5 equiv) in portions at 0° C. The resulting mixture was stirred for 1 h at 0° C. The resulting mixture was concentrated under reduced pressure. The residue was purified by silica gel column chromatography, eluted with PE/THF (3:1) to afford tert-butyl (2S)-2-{[(1S)-2-{4-[3-(2-butoxyethyl)-2-oxo-1,3-benzoxazol-5-yl]-2-fluorophenyl}-1-cyanoethyl]carbamoyl}-6-methoxy-5-methyl-1,4-oxazepane-4-carboxylate (180 mg, 77.87%) as a light yellow solid. LCMS (ES) [M+H]⁺ m/z: 669.

### Step 2. Synthesis of (2S)—N-[(1S)-2-{4-[3-(2-butoxyethyl)-2-oxo-1,3-benzoxazol-5-yl]-2-fluorophenyl}-1-cyanoethyl]-6-methoxy-5-methyl-1,4-oxazepane-2-carboxamide

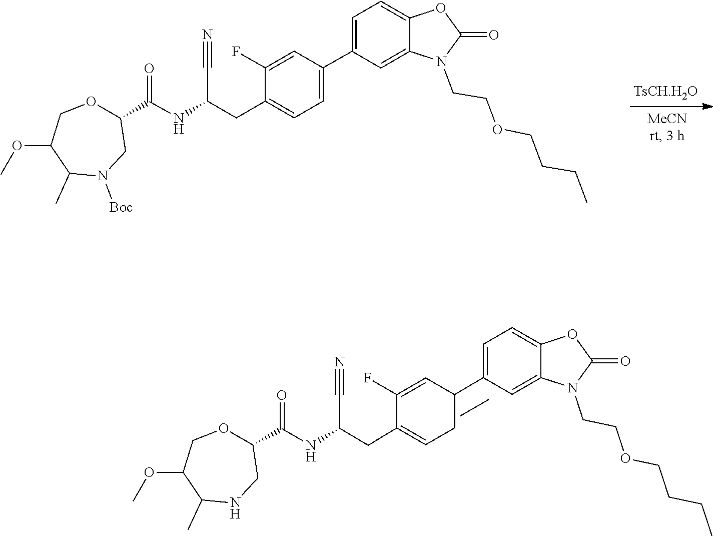

To a stirred solution of tert-butyl (2S)-2-{[(1S)-2-{4-[3-(2-butoxyethyl)-2-oxo-1,3-benzoxazol-5-yl]-2-fluorophenyl}-1-cyanoethyl]carbamoyl}-6-methoxy-5-methyl-1,4-oxazepane-4-carboxylate (180 mg, 0.269 mmol, 1.0 equiv) in MeCN (3 mL) was added TsOH·H₂O (153 mg, 0.807 mmol, 3.0 equiv). The resulting mixture was stirred for 3 h at room temperature. The mixture was purified by reversed-phase flash chromatography with the following conditions: column, C18 silica gel; mobile phase, MeCN in Water (0.1% NH3·H2O), 10% to 60% gradient in 15 min; detector, UV 254 nm. This resulted in Compound E1 (90 mg, 58.80%) as a white solid. LCMS (ES) [M+H]⁺ m/z: 569.

### Step 3. Synthesis of Compound E2, Compound E3, and Compound E4

Compound E1 (90 mg, 0.158 mmol) was purified by Prep-HPLC with the following conditions (Column: XA-CHIRALPAK IH, 3*25 cm, 5 m; Mobile Phase A: Hex:DCM=5:1-HPLC, Mobile Phase B: ETOH (0.1% 2M NH₃-MEOH); Flow rate: 35 mL/min; Gradient: isocratic 20; Wave Length: 254 nm; RT1(min): 6.4; RT2(min): 8.6; Sample Solvent: EtOH:DCM=1:1-HPLC; Injection Volume: 1 mL; Number Of Runs: 4) to afford Compound E2 (13 mg, 14.44%), Compound E3 (50 mg, 55.56%), and Compound E4 (12 mg, 13.33%) as a white solid. LCMS (ES) [M+H]⁺ m/z: 569.

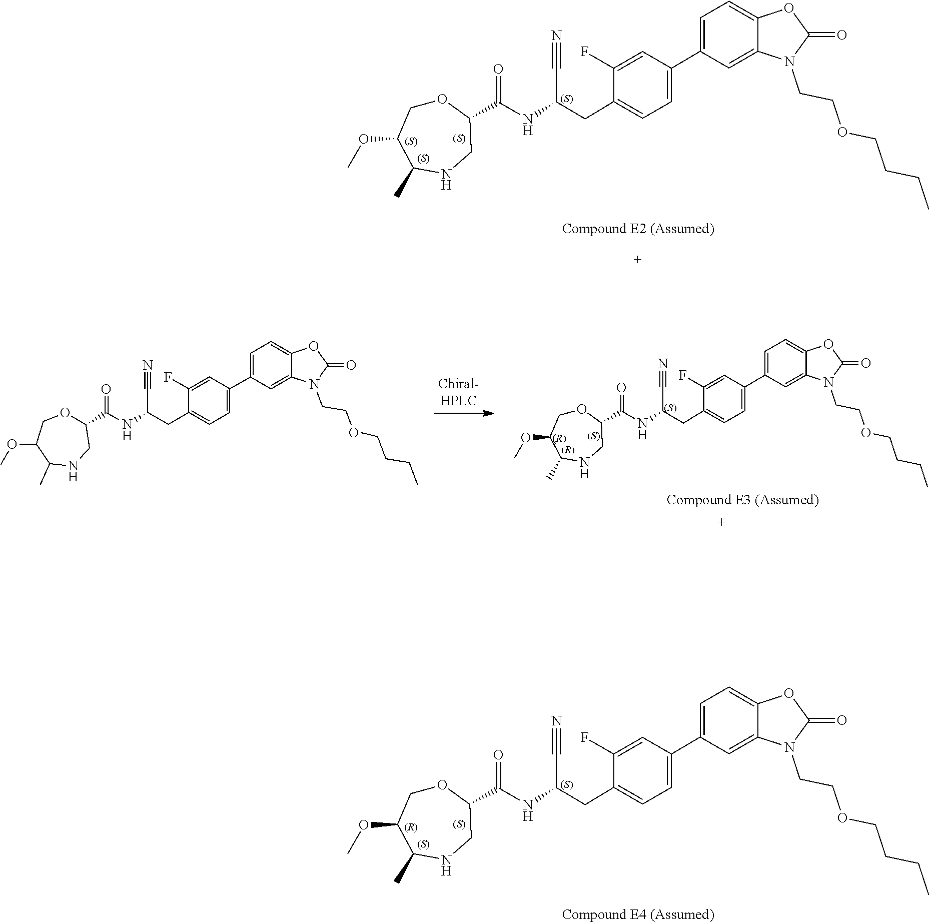

Compound E2: ¹H NM/R (300 MHz, DMSO-d₆) δ 8.72 (d, J=8.6 Hz, 1H), 7.67 (d, J=1.8 Hz, 1H), 7.59-7.46 (m, 2H), 7.50-7.35 (m, 3H), 5.02 (q, J=8.1 Hz, 1H), 4.03 (dt, J=9.9, 4.0 Hz, 3H), 3.82 (d, J=4.1 Hz, 2H), 3.69 (t, J=5.2 Hz, 2H), 3.37 (t, J=6.3 Hz, 2H), 3.31-3.29 (m, 4H), 3.27-3.20 (in, 1H), 3.16 (dd, J=13.7, 8.6 Hz, 1H), 3.04 (dd, J=14.4, 3.0 Hz, 1H), 2.89-2.77 (in, 1H), 2.37 (dd, J=14.5, 9.8 Hz, 1H), 1.98 (brs, 1H), 1.43-1.28 (m, 2H), 1.25-1.07 (m, 2H), 1.00 (d, J=6.7 Hz, 3H), 0.71 (t, J=7.3 Hz, 3H).

Compound E3: ¹H NMR (300 MHz, DMSO-d₆) δ 8.61 (d, J=8.4 Hz, 1H), 7.69 (d, J=1.8 Hz, 1H), 7.61-7.37 (m, 5H), 5.08 (q, J=8.0 Hz, 1H), 4.10 (ddd, J=12.5, 6.6, 3.5 Hz, 4H), 3.71 (t, J=5.2 Hz, 2H), 3.59 (dd, J=13.4, 1.7 Hz, 1H), 3.39-3.13 (m, 8H), 3.02-2.83 (m, 2H), 2.67 (dd, J=14.5, 7.2 Hz, 1H), 1.98 (s, 1H), 1.37 (dq, J=8.3, 6.4 Hz, 2H), 1.27-1.09 (m, 2H), 1.01 (d, J=6.7 Hz, 3H), 0.74 (t, J=7.3 Hz, 3H).

Compound E4: ¹H NMR (300 MHz, DMSO-d₆) δ 8.63 (d, J=8.5 Hz, 1H), 7.66 (d, J=1.7 Hz, 1H), 7.59-7.35 (m, 5H), 5.02 (p, J=7.9 Hz, 1H), 4.04 (t, J=5.3 Hz, 2H), 3.97 (t, J=4.0 Hz, 1H), 3.85 (dd, J=12.7, 3.2 Hz, 1H), 3.69 (t, J=5.2 Hz, 2H), 3.53 (dd, J=12.7, 9.0 Hz, 1H), 3.37 (t, J=6.3 Hz, 2H), 3.31 (s, 3H), 3.28-3.14 (m, 2H), 3.07-2.86 (m, 2H), 2.79 (td, J=8.6, 3.1 Hz, 1H), 2.39 (p, J=6.4 Hz, 1H), 1.95 (s, 1H), 1.43-1.28 (m, 2H), 1.25-1.07 (m, 2H), 0.99 (d, J=6.2 Hz, 3H), 0.71 (t, J=7.3 Hz, 3H).

### Example 64: Synthesis of (2S)—N-[(1S)-1-cyano-2-(2-fluoro-4-{1′-methyl-2,3-dihydrospiro[indene-1,4′-piperidin]-6-yl}phenyl)ethyl]-6-methoxy-5-methyl-1,4-oxazepane-2-carboxamide (Compound E6), (2S,5S,6S)—N-[(1S)-1-cyano-2-(2-fluoro-4-{1′-methyl-2,3-dihydrospiro[indene-1,4′-piperidin]-6-yl}phenyl)ethyl]-6-methoxy-5-methyl-1,4-oxazepane-2-carboxamide (Compound E7), (2S,5R,6R)—N-[(1S)-1-cyano-2-(2-fluoro-4-{1′-methyl-2,3-dihydrospiro[indene-1,4′-piperidin]-6-yl}phenyl)ethyl]-6-methoxy-5-methyl-1,4-oxazepane-2-carboxamide (Compound E8), and (2S,5S,6R)—N-[(1S)-1-cyano-2-(2-fluoro-4-{1′-methyl-2,3-dihydrospiro[indene-1,4′-piperidin]-6-yl}phenyl)ethyl]-6-methoxy-5-methyl-1,4-oxazepane-2-carboxamide (Compound E9)

### Step 1. Synthesis of tert-butyl (2S)-2-{[(1S)-1-cyano-2-(2-fluoro-4-{1′-methyl-2,3-dihydrospiro[indene-1,4′-piperidin]-6-yl}phenyl)ethyl]carbamoyl}-6-methoxy-5-methyl-1,4-oxazepane-4-carboxylate

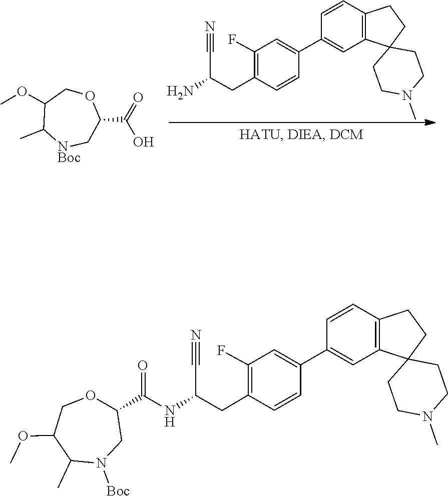 [SMILES: COC1CO[C@H](C(=O)O)CN(C(=O)OC(C)(C)C)C1C ; CN1CCC2(CCc3ccc(-c4ccc(C[C@H](N)C#N)c(F)c4)cc32)CC1 ; COC1CO[C@H](C(=O)N[C@H](C#N)Cc2ccc(-c3ccc4c(c3)C3(CC4)CCN(C)CC3)cc2F)CN(C(=O)OC(C)(C)C)C1C]

To a stirred solution of (2S)-4-(tert-butoxycarbonyl)-6-methoxy-5-methyl-1,4-oxazepane-2-carboxylic acid (100 mg, 0.346 mmol, 1.0 equiv) and (2S)-2-amino-3-(2-fluoro-4-{1′-methyl-2,3-dihydrospiro[indene-1,4′-piperidin]-6-yl}phenyl)propanenitrile (125 mg, 0.346 mmol, 1.0 equiv) in DCM (3 mL) were added DIEA (134 mg, 1.038 mmol, 3.0 equiv) and HATU (197 mg, 0.519 mmol, 1.5 equiv) in portions at 0° C. The resulting mixture was stirred for 2 h at 0° C. The resulting mixture was concentrated under reduced pressure. The residue was purified by silica gel column chromatography, eluted with CH₂Cl₂/MeOH (10:1) to afford tert-butyl (2S)-2-{[(1S)-1-cyano-2-(2-fluoro-4-{1′-methyl-2,3-dihydrospiro[indene-1,4′-piperidin]-6-yl}phenyl)ethyl]carbamoyl}-6-methoxy-5-methyl-1,4-oxazepane-4-carboxylate (150 mg, 68.37%) as a light brown solid. LCMS (ES) [M+H]⁺ m/z: 635.

### Step 2. Synthesis of 2S)—N-[(1S)-1-cyano-2-(2-fluoro-4-{1′-methyl-2,3-dihydrospiro[indene-1,4′-piperidin]-6-yl}phenyl)ethyl]-6-methoxy-5-methyl-1,4-oxazepane-2-carboxamide

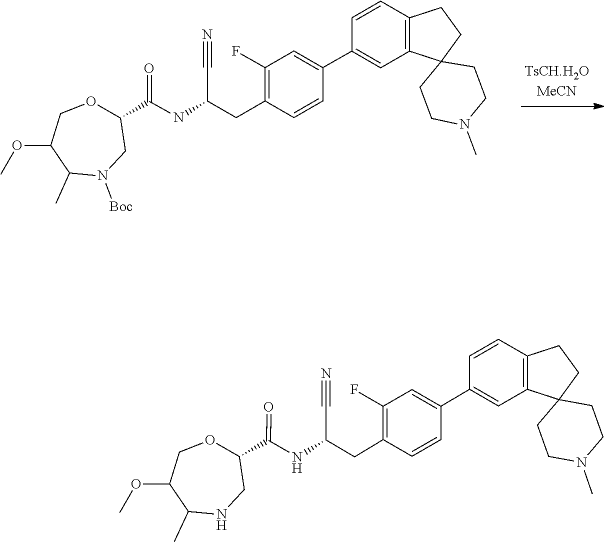

To a stirred solution of tert-butyl (2S)-2-{[(1S)-1-cyano-2-(2-fluoro-4-{1′-methyl-2,3-dihydrospiro[indene-1,4′-piperidin]-6-yl}phenyl)ethyl]carbamoyl}-6-methoxy-5-methyl-1,4-oxazepane-4-carboxylate (150 mg, 0.236 mmol, 1.0 equiv) in MeCN (3 mL) was added TsOH·H₂O (134.84 mg, 0.708 mmol, 3.0 equiv). The resulting mixture was stirred for 3 h at room temperature. The residue was purified by reversed-phase flash chromatography with the following conditions: column, C18 silica gel; mobile phase, MeCN in Water (0.1% NH₃·H₂O), 10% to 75% gradient in 15 min; detector, UV 254 nm. This resulted in Compound E6 (65 mg, 51.45%) as a white solid. LCMS (ES) [M+H]⁺ m/z: 535.

### Step 3. Synthesis of Compound E7, Compound E8, and Compound E9

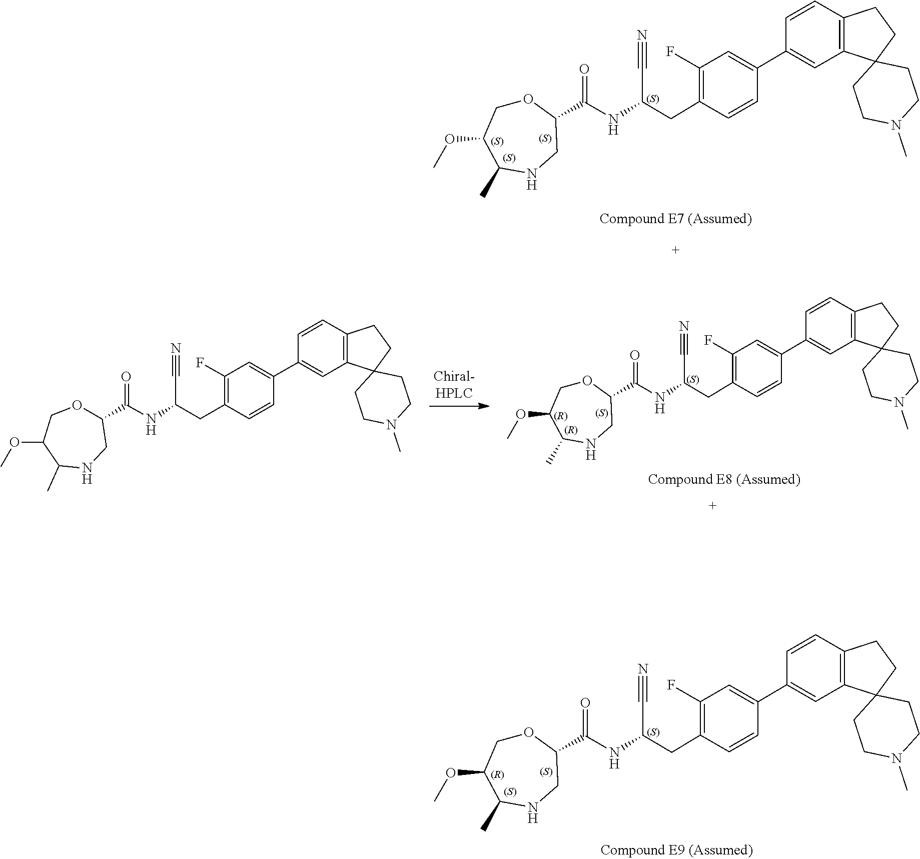

(2S)—N-[(1S)-1-cyano-2-(2-fluoro-4-{1′-methyl-2,3-dihydrospiro[indene-1,4′-piperidin]-6-yl}phenyl)ethyl]-6-methoxy-5-methyl-1,4-oxazepane-2-carboxamide (65 mg, 0.122 mmol) was purified by Prep-HPLC with the following conditions (Column: XA-CHIRALPAK IH, 3*25 cm, 5 m; Mobile Phase A: Hex:DCM=5:1-HPLC, Mobile Phase B: IPA-HPLC; Flow rate: 35 mL/min; Gradient: isocratic 10; Wave Length: 254 nm; RT1(min): 7.1; RT2(min): 10.2; Sample Solvent: IPA-HPLC; Injection Volume: 0.5 mL; Number Of Runs: 14) to afford Compound E7 (5.0 mg, 7.69%), Compound E8 (17 mg, 26.15%) and Compound E9 (3.8 mg, 5.85%) as a white solid. LCMS (ES) [M+H]⁺ m/z: 535.

Compound E7: ¹H NMR (400 MHz, DMSO-d₆) δ 8.72 (d, J=8.5 Hz, 1H), 7.55-7.44 (m, 4H), 7.41 (t, J=7.9 Hz, 1H), 7.28 (d, J=7.8 Hz, 1H), 5.04 (q, J=8.2 Hz, 1H), 4.04 (dd, J=10.1, 3.0 Hz, 1H), 3.84 (d, J=4.1 Hz, 2H), 3.33-3.24 (m, 5H), 3.17 (dd, J=13.8, 8.7 Hz, 1H), 3.06 (dd, J=14.9, 3.1 Hz, 1H), 2.87 (q, J=7.1 Hz, 3H), 2.75 (d, J=11.0 Hz, 2H), 2.39 (dd, J=14.5, 9.8 Hz, 1H), 2.24 (s, 3H), 2.10 (t, J=11.9 Hz, 2H), 2.04-1.89 (m, 4H), 1.46 (d, J=12.6 Hz, 2H), 1.03 (d, J=6.7 Hz, 3H).

Compound E8: ¹H NMR (400 MHz, DMSO-d₆) δ 8.60 (d, J=8.4 Hz, 1H), 7.48 (td, J=14.9, 13.8, 9.6 Hz, 5H), 7.28 (d, J=7.8 Hz, 1H), 5.07 (q, J=8.1 Hz, 1H), 4.15-4.06 (m, 2H), 3.60 (d, J=13.3 Hz, 1H), 3.35 (d, J=1.2 Hz, 3H), 3.27 (dd, J=13.8, 7.6 Hz, 1H), 3.23-3.14 (m, 2H), 3.01-2.84 (m, 4H), 2.78 (d, J=11.3 Hz, 2H), 2.69 (dd, J=14.6, 7.1 Hz, 1H), 2.26 (s, 3H), 2.14 (t, J=12.0 Hz, 2H), 2.04-1.89 (m, 4H), 1.47 (d, J=12.7 Hz, 2H), 1.01 (d, J=6.6 Hz, 3H).

Compound E9: ¹H NMR (400 MHz, DMSO-d₆) δ 8.63 (d, J=8.4 Hz, 1H), 7.55-7.42 (m, 5H), 7.28 (d, J=7.8 Hz, 1H), 5.05 (q, J=8.0 Hz, 1H), 3.99 (d, J=4.2 Hz, 1H), 3.87 (dd, J=12.6, 3.2 Hz, 1H), 3.55 (dd, J=12.6, 8.9 Hz, 1H), 3.32-3.20 (m, 5H), 3.03 (dd, J=15.2, 3.1 Hz, 1H), 2.94 (dd, J=15.0, 4.8 Hz, 1H), 2.88 (t, J=7.2 Hz, 2H), 2.85-2.75 (m, 3H), 2.42 (p, J=6.5 Hz, 1H), 2.27 (s, 3H), 2.19-2.14 (m, 2H), 2.04-1.89 (m, 4H), 1.48 (d, J=12.7 Hz, 2H), 1.01 (d, J=6.3 Hz, 3H).

### Example 65: Synthesis of (2S,6S)—N-[(1S)-1-cyano-2-{4′-cyano-[1,1′-biphenyl]-4-yl}ethyl]-6-methoxy-1,4-oxazocane-2-carboxamide (Compound F1)

### Step 1. Synthesis of tert-butyl (2S,6S)-2-{[(1S)-1-cyano-2-{4′-cyano-[1,1′-biphenyl]-4-yl}ethyl]carbamoyl}-6-methoxy-1,4-oxazocane-4-carboxylate

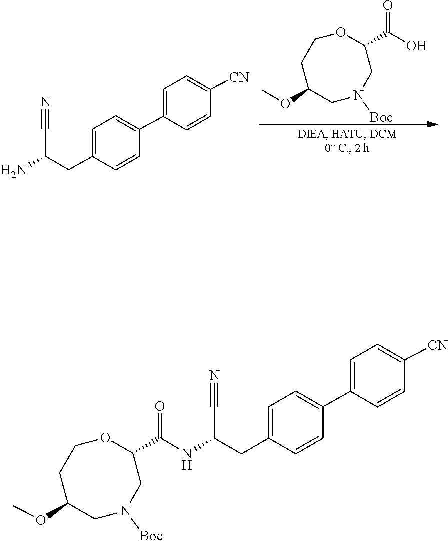

To a stirred solution of 4′-[(2S)-2-amino-2-cyanoethyl]-[1,1′-biphenyl]-4-carbonitrile (82 mg, 0.332 mmol, 1.2 equiv) and (2S,6S)-4-(tert-butoxycarbonyl)-6-methoxy-1,4-oxazocane-2-carboxylic acid (80 mg, 0.277 mmol, 1.0 equiv) in DCM (2 mL) were added DIEA (107 mg, 0.831 mmol, 3.0 equiv) and HATU (126 mg, 0.332 mmol, 1.2 equiv) in portions at 0° C. under nitrogen atmosphere. The resulting mixture was stirred at 0° C. for 2 h. The residue was purified by silica gel column chromatography, eluted with PE/THF (2:1) to afford tert-butyl (2S,6S)-2-{[(1S)-1-cyano-2-{4′-cyano-[1,1′-biphenyl]-4-yl}ethyl]carbamoyl}-6-methoxy-1,4-oxazocane-4-carboxylate (110 mg, 76.7%) as an off-white solid. LCMS (ES) [M+1]⁺ m/z: 519.

### Step 2. Synthesis of (2S,6S)—N-[(1S)-1-cyano-2-{4′-cyano-[1,1′-biphenyl]-4-yl}ethyl]-6-methoxy-1,4-oxazocane-2-carboxamide

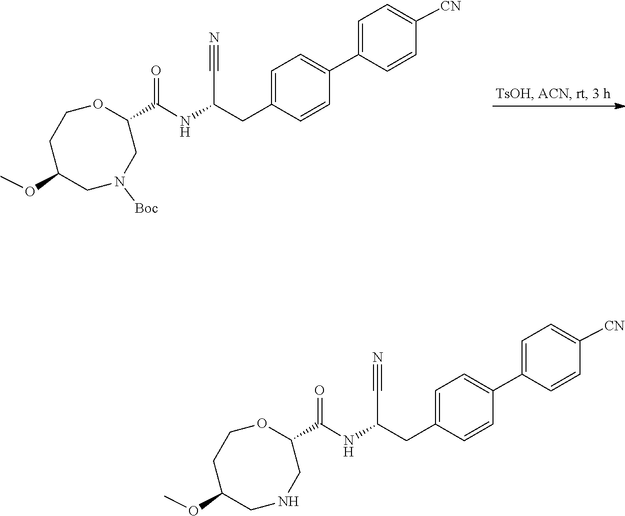 [SMILES: CO[C@H]1CCO[C@H](C(=O)N[C@H](C#N)Cc2ccc(-c3ccc(C#N)cc3)cc2)CN(C(=O)OC(C)(C)C)C1 ; CO[C@H]1CCO[C@H](C(=O)N[C@H](C#N)Cc2ccc(-c3ccc(C#N)cc3)cc2)CNC1]

To a stirred solution of tert-butyl (2S,6S)-2-{[(1S)-1-cyano-2-{4′-cyano-[1,1′-biphenyl]-4-yl}ethyl]carbamoyl}-6-methoxy-1,4-oxazocane-4-carboxylate (100 mg, 0.193 mmol, 1.0 equiv) in ACN (2 mL) was added TsOH (99 mg, 0.579 mmol, 3.0 equiv). The resulting mixture was stirred at room temperature for 3 h. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound F1 (30 mg, 37.1%) as a white solid. LCMS (ES) [M+1]⁺ m/z: 419. ¹H NMR (300 MHz, DMSO-d6) δ 8.64 (d, J=8.6 Hz, 1H), 7.98-7.83 (m, 4H), 7.77-7.67 (m, 2H), 7.45-7.37 (m, 2H), 5.08-4.94 (m, 1H), 3.90 (dd, J=10.1, 2.6 Hz, 1H), 3.82 (td, J=11.8, 3.0 Hz, 1H), 3.68 (dt, J=12.0, 3.9 Hz, 1H), 3.30-3.09 (m, 7H), 2.96 (dd, J=13.6, 2.6 Hz, 1H), 2.66 (dd, J=14.7, 3.7 Hz, 1H), 2.34-2.01 (m, 3H), 1.62 (dd, J=14.6, 3.6 Hz, 1H).

### Example 66: Synthesis of (2S,6S)—N-[(1S)-2-{4-[3-(2-butoxyethyl)-2-oxo-1,3-benzoxazol-5-yl]phenyl}-1-cyanoethyl]-6-methoxy-1,4-oxazocane-2-carboxamide (Compound F2)

### Step 1. Synthesis of tert-butyl (2S,6S)-2-{[(1S)-2-{4-[3-(2-butoxyethyl)-2-oxo-1,3-benzoxazol-5-yl]phenyl}-1-cyanoethyl]carbamoyl}-6-methoxy-1,4-oxazocane-4-carboxylate

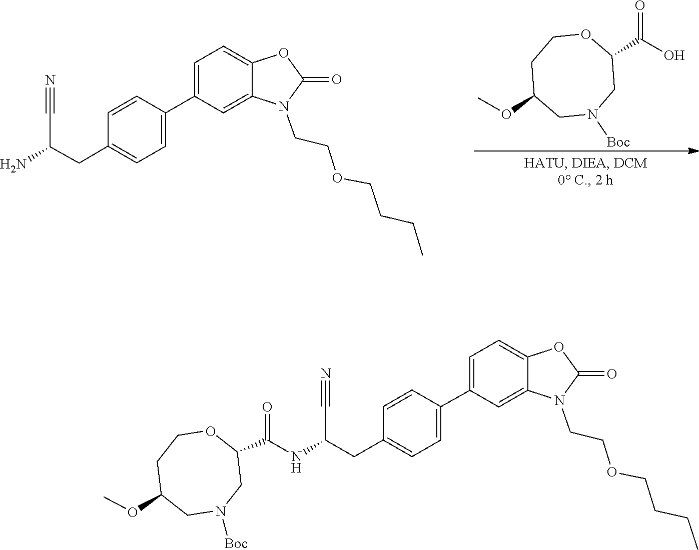 [SMILES: CCCCOCCn1c(=O)oc2ccc(-c3ccc(C[C@H](N)C#N)cc3)cc21 ; CO[C@H]1CCO[C@H](C(=O)O)CN(C(=O)OC(C)(C)C)C1 ; CCCCOCCn1c(=O)oc2ccc(-c3ccc(C[C@@H](C#N)NC(=O)[C@@H]4CN(C(=O)OC(C)(C)C)C[C@@H](OC)CCO4)cc3)cc21]

To a solution of (2S)-2-amino-3-{4-[3-(2-butoxyethyl)-2-oxo-1,3-benzoxazol-5-yl]phenyl}propanenitrile (120 mg, 0.32 mmol, 1.0 equiv), 2S,6S)-4-(tert-butoxycarbonyl)-6-methoxy-1,4-oxazocane-2-carboxylic acid (92 mg, 0.32 mmol, 1.0 equiv) and DIEA (123 mg, 0.95 mmol, 3.0 equiv) in DCM (3 mL) was added HATU (180 mg, 0.47 mmol, 1.5 equiv) at 0° C. The resulting mixture was stirred for 2 h at 0° C. The reaction was concentrated. The residue was purified by silica gel column chromatography, eluted with PE/THF (1:1) to afford tert-butyl (2S,6S)-2-{[(1S)-2-{4-[3-(2-butoxyethyl)-2-oxo-1,3-benzoxazol-5-yl]phenyl}-1-cyanoethyl]carbamoyl}-6-methoxy-1,4-oxazocane-4-carboxylate (130 mg, 63.17%) as a light yellow semi-solid. LCMS (ES, m/z): [M+H]⁺: 651.

### Step 2. Synthesis of (2S,6S)—N-[(1S)-2-{4-[3-(2-butoxyethyl)-2-oxo-1,3-benzoxazol-5-yl]phenyl}-1-cyanoethyl]-6-methoxy-1,4-oxazocane-2-carboxamide

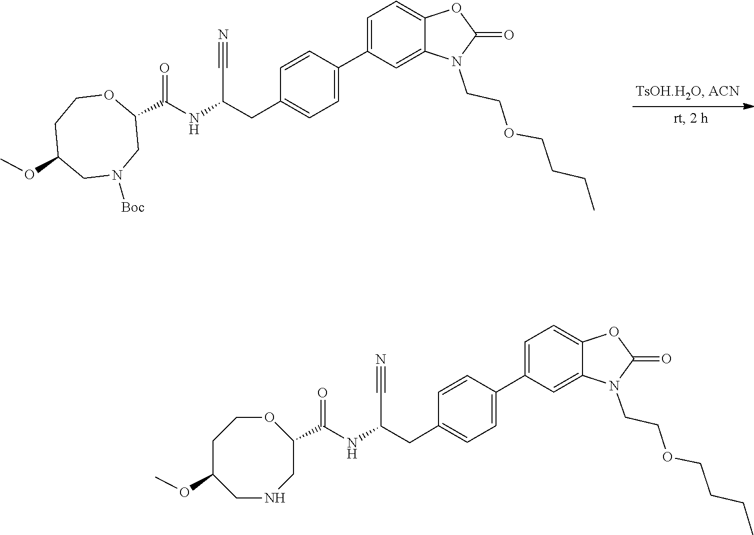

Into a 8 mL vial were added tert-butyl (2S,6S)-2-{[(1S)-2-{4-[3-(2-butoxyethyl)-2-oxo-1,3-benzoxazol-5-yl]phenyl}-1-cyanoethyl]carbamoyl}-6-methoxy-1,4-oxazocane-4-carboxylate (120 mg, 0.18 mmol, 1.0 equiv), TsOH·H₂O (95 mg, 0.55 mmol, 3.0 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 80% gradient in 10 min; detector, UV 254 nm. This resulted in Compound F2 (30 mg, 29.55%) as a white solid. LCMS (ES, m/z): [M+H]⁺: 551.2. ¹H NMR (400 MHz, DMSO-d₆) δ 8.61 (d, J=8.5 Hz, 1H), 7.66-7.58 (m, 3H), 7.42-7.32 (m, 4H), 4.97 (q, J=8.2 Hz, 1H), 4.05 (t, J=5.3 Hz, 2H), 3.90 (dd, J=10.1, 2.6 Hz, 1H), 3.86-3.75 (m, 1H), 3.73-3.63 (m, 3H), 3.38 (t, J=6.4 Hz, 2H), 3.25-3.10 (m, 7H), 2.98 (dd, J=13.7, 2.6 Hz, 1H), 2.67 (dd, J=14.7, 3.7 Hz, 1H), 2.27-2.05 (m, 3H), 1.62 (dd, J=14.7, 3.6 Hz, 1H), 1.42-1.30 (m, 2H), 1.25-1.10 (m, 2H), 0.73 (t, J=7.4 Hz, 3H).

### Example 67: Synthesis of (2S,6S)—N-[(1S)-1-cyano-2-[5-(3-methyl-2-oxo-1,3-benzoxazol-5-yl)thiophen-2-yl]ethyl]-6-methoxy-1,4-oxazocane-2-carboxamide (Compound F3)

### Step 1. Synthesis of tert-butyl (2S,6S)-2-{[(1S)-1-cyano-2-[5-(3-methyl-2-oxo-1,3-benzoxazol-5-yl)thiophen-2-yl]ethyl]carbamoyl}-6-methoxy-1,4-oxazocane-4-carboxylate

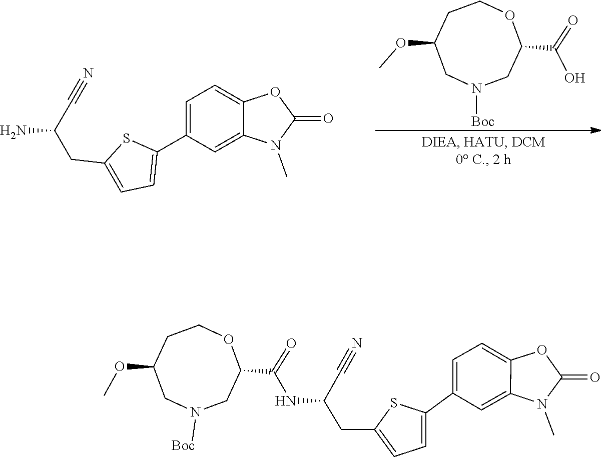 [SMILES: Cn1c(=O)oc2ccc(-c3ccc(C[C@H](N)C#N)s3)cc21 ; CO[C@H]1CCO[C@H](C(=O)O)CN(C(=O)OC(C)(C)C)C1 ; CO[C@H]1CCO[C@H](C(=O)N[C@H](C#N)Cc2ccc(-c3ccc4oc(=O)n(C)c4c3)s2)CN(C(=O)OC(C)(C)C)C1]

To a stirred solution of (2S,6S)-4-(tert-butoxycarbonyl)-6-methoxy-1,4-oxazocane-2-carboxylic acid (80 mg, 0.277 mmol, 1.0 equiv) and (2S)-2-amino-3-[5-(3-methyl-2-oxo-1,3-benzoxazol-5-yl)thiophen-2-yl]propanenitrile (99 mg, 0.332 mmol, 1.2 equiv) in DCM (2 mL) were added DIEA (107 mg, 0.831 mmol, 3.0 equiv) and HATU (126 mg, 0.332 mmol, 1.2 equiv) in portions at 0° C. under nitrogen atmosphere. The resulting mixture was stirred at 0° C. for 2 h. The residue was purified by silica gel column chromatography, eluted with PE/THF (1:1) to afford tert-butyl (2S,6S)-2-{[(1S)-1-cyano-2-[5-(3-methyl-2-oxo-1,3-benzoxazol-5-yl)thiophen-2-yl]ethyl]carbamoyl}-6-methoxy-1,4-oxazocane-4-carboxylate (110 mg, 69.7%) as an off-white solid. LCMS (ES) [M+1]⁺ m/z: 571.

### Step 2. Synthesis of (2S,6S)—N-[(1S)-1-cyano-2-[5-(3-methyl-2-oxo-1,3-benzoxazol-5-yl)thiophen-2-yl]ethyl]-6-methoxy-1,4-oxazocane-2-carboxamide

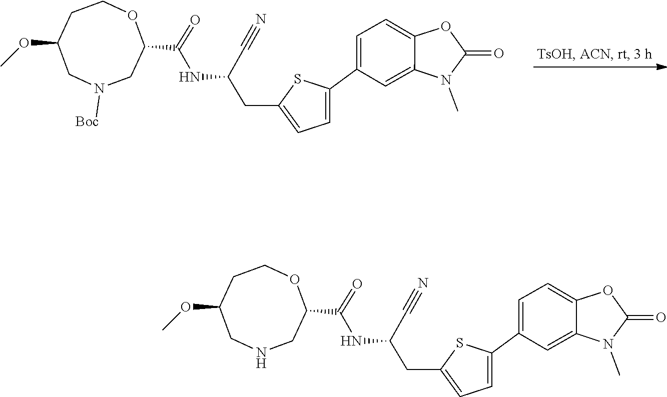 [SMILES: CO[C@H]1CCO[C@H](C(=O)N[C@H](C#N)Cc2ccc(-c3ccc4oc(=O)n(C)c4c3)s2)CN(C(=O)OC(C)(C)C)C1 ; CO[C@H]1CCO[C@H](C(=O)N[C@H](C#N)Cc2ccc(-c3ccc4oc(=O)n(C)c4c3)s2)CNC1]

To a stirred solution of tert-butyl (2S,6S)-2-{[(1S)-1-cyano-2-[5-(3-methyl-2-oxo-1,3-benzoxazol-5-yl)thiophen-2-yl]ethyl]carbamoyl}-6-methoxy-1,4-oxazocane-4-carboxylate (100 mg, 0.175 mmol, 1.0 equiv) in ACN (2 mL) was added TsOH (90 mg, 0.525 mmol, 3.0 equiv). The resulting mixture was stirred at room temperature for 3 h. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound F3 (30 mg, 36.3%) as a white solid. LCMS (ES) [M+1]⁺ m/z: 471. ¹H NMR (300 MHz, DMSO-d6) δ 8.71 (d, J=8.6 Hz, 1H), 7.51 (dd, J=1.6, 0.7 Hz, 1H), 7.42-7.28 (m, 3H), 6.99 (d, J=3.7 Hz, 1H), 5.05-4.90 (m, 1H), 3.96 (dd, J=10.1, 2.6 Hz, 1H), 3.90-3.77 (m, 1H), 3.76-3.65 (m, 1H), 3.48-3.33 (m, 5H), 3.29-3.12 (m, 5H), 3.05 (dd, J=13.7, 2.6 Hz, 1H), 2.70 (dd, J=14.7, 3.7 Hz, 1H), 2.36-2.11 (m, 3H), 1.64 (dd, J=14.5, 3.5 Hz, 1H).

### Example 68: Synthesis of (2S,7R)—N—((S)-1-cyano-2-(4′-cyano-3-fluoro-[1,1′-biphenyl]-4-yl)ethyl)-7-methoxy-1,4-oxazocane-2-carboxamide (Compound F4)

### Step 1. Synthesis of tert-butyl (2S,7R)-2-(((S)-1-cyano-2-(4′-cyano-3-fluoro-[1,1′-biphenyl]-4-yl)ethyl)carbamoyl)-7-methoxy-1,4-oxazocane-4-carboxylate

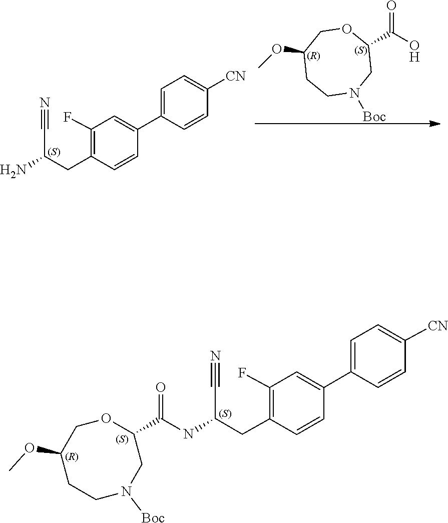

To a solution of 4′-[(2S)-2-amino-2-cyanoethyl]-3′-fluoro-[1,1′-biphenyl]-4-carbonitrile (650 mg, 2.45 mmol, 1.0 equiv), (2S,7R)-4-(tert-butoxycarbonyl)-7-methoxy-1,4-oxazocane-2-carboxylic acid (780 mg, 2.70 mmol, 1.1 equiv), DIEA (950 mg, 7.35 mmol, 3.0 equiv) in DCM (15 mL) was added HATU (1118 mg, 2.94 mmol, 1.2 equiv) at 0° C. The resulting mixture was stirred at 0° C. for 2 h. The reaction was quenched with water (20 mL) at 0° C. The aqueous layer was extracted with CH₂Cl₂ (20 mL×1). The resulting mixture was dried over anhydrous sodium sulfate. After filtration, the filtrate was concentrated under vacuum. The residue was purified by silica gel column chromatography, eluted with PE/EA (1:2) to afford tert-butyl (2S,7R)-2-{[(1S)-1-cyano-2-{4′-cyano-3-fluoro-[1,1′-biphenyl]-4-yl}ethyl]carbamoyl}-7-methoxy-1,4-oxazocane-4-carboxylate (1 g, 76%) as a white solid. LCMS (ES) [M+1]⁺ m/z: 537.

### Step 2. Synthesis of (2S,7R)—N—((S)-1-cyano-2-(4′-cyano-3-fluoro-[1,1′-biphenyl]-4-yl)ethyl)-7-methoxy-1,4-oxazocane-2-carboxamide

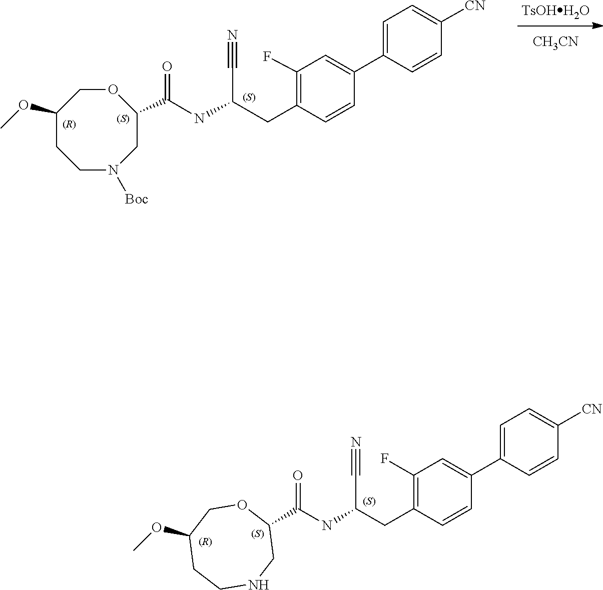

Into a 100 mL round-bottom flask were added tert-butyl (2S,7R)-2-{[(1S)-1-cyano-2-{4′-cyano-3-fluoro-[1,1′-biphenyl]-4-yl}ethyl]carbamoyl}-7-methoxy-1,4-oxazocane-4-carboxylate (1 g, 1.86 mmol, 1.0 equiv), CH₃CN (20 mL) and TsOH·H2O (1.06 g, 5.59 mmol, 3.0 equiv) at room temperature. The resulting mixture was stirred at room temperature for 2 h. The reaction solution was purified by reverse phase flash with the following conditions: column, mobile phase, MeCN in water (0.1% NH₃·H₂O), 10% to 70% gradient in 10 min; detector, UV 254 nm. This resulted in Compound F4 (287 mg, 35%) as a white solid. LCMS (ES) [M+1]⁺ m/z: 437.1. ¹H NMR (300 MHz, DMSO-d₆) δ 8.74 (d, J=8.6 Hz, 1H), 7.94 (s, 4H), 7.72-7.62 (m, 1H), 7.60 (d, J=1.8 Hz, 1H), 7.50 (t, J=7.9 Hz, 1H), 5.07 (q, J=8.3 Hz, 1H), 4.10 (dd, J=11.5, 4.2 Hz, 1H), 3.92 (dd, J=9.1, 3.0 Hz, 1H), 3.58 (dd, J=11.5, 8.5 Hz, 1H), 3.32-3.14 (m, 6H), 3.01 (dd, J=14.4, 3.5 Hz, 2H), 2.53-2.48 (m, 1H), 2.34 (dd, J=14.4, 9.1 Hz, 1H), 1.91 (t, J=11.4 Hz, 1H), 1.60 (dd, J=14.6, 8.0 Hz, 1H).

### Example 69: Synthesis of (S)—N—((S)-1-cyano-2-(4′-(pentafluoro-16-sulfaneyl)-[1,1′-biphenyl]-4-yl)ethyl)-1,4-oxazepane-2-carboxamide (Compound 6)

### Step 1. Synthesis of tert-butyl (2S)-2-{[(1S)-1-cyano-2-{4′-[(4-methylpiperazin-1-yl)methyl]-[1,1′-biphenyl]-4-yl}ethyl]carbamoyl}-1,4-oxazepane-4-carboxylate

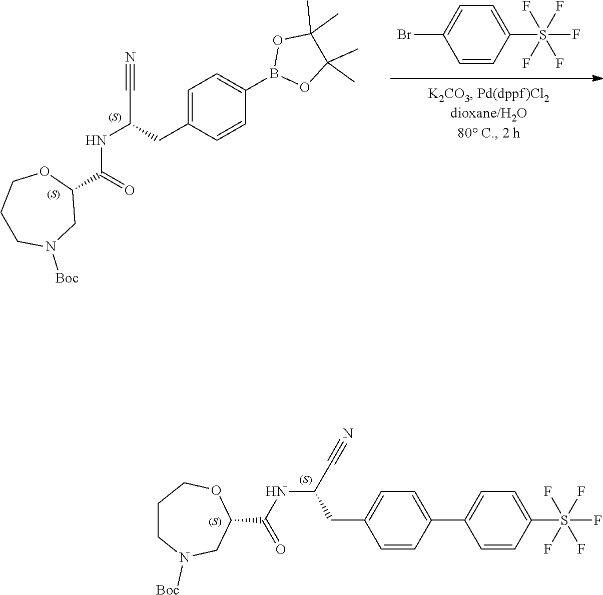

To a solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1.0 equiv), 1-bromo-4-(pentafluoro-lambda6-sulfanyl)benzene (68 mg, 0.24 mmol, 1.0 equiv) in 1,4-dioxane (5 mL) and H₂O (0.5 mL), K₂CO₃ (66 mg, 0.48 mmol, 2.0 equiv) and Pd(dppf)Cl₂ (18 mg, 0.02 mmol, 0.1 equiv) were added in sequence. The mixture was heated to 80° C. and stirred for 2 h under nitrogen gas atmosphere. The reaction was cooled to room temperature, concentrated under reduced pressure. The residue was purified by silica gel column chromatography, eluted with PE/THF (1:1) to afford tert-butyl (2S)-2-{[(1S)-1-cyano-2-[4′-(pentafluoro-lambda6-sulfanyl)-[1,1′-biphenyl]-4-yl]ethyl]carbamoyl}-1,4-oxazepane-4-carboxylate (78 mg, 56.4%) as yellow oil. LCMS (ES, m/z): [M+H]⁺: 576.

### Step 2. Synthesis of (2S)—N-[(1S)-1-cyano-2-{4′-[(4-methylpiperazin-1-yl)methyl]-[1,1′-biphenyl]-4-yl}ethyl]-1,4-oxazepane-2-carboxamide

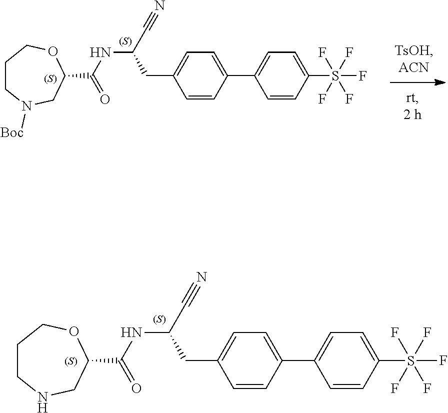

Into a 25 mL round-bottom flask were added tert-butyl (2S)-2-{[(1S)-1-cyano-2-[4′-(pentafluoro-lambda6-sulfanyl)-[1,1′-biphenyl]-4-yl]ethyl]carbamoyl}-1,4-oxazepane-4-carboxylate (78 mg, 0.13 mmol, 1.0 equiv), TsOH (70 mg, 0.40 mmol, 3.0 equiv) and ACN (3 mL) at room temperature. The resulting mixture was stirred for additional 2 h at room temperature. The reaction solution was purified by reversed-phase flash chromatography with the following conditions: column, C18 silica gel −120 g; mobile phase, MeCN in Water (0.1% NH₃·H₂O), 10% to 80% gradient in 10 min; detector, UV 254 nm. This resulted in Compound 6 (24.3 mg, 37.7%) as yellow oil. LCMS (ES, m/z): [M+H]⁺: 476.2. ¹H NMR (400 MHz, DMSO-d₆) δ 8.61 (d, J=8.6 Hz, 1H), 7.98 (d, J=8.8 Hz, 2H), 7.89 (d, J=8.6 Hz, 2H), 7.71 (d, J=8.0 Hz, 2H), 7.44 (d, J=8.0 Hz, 2H), 5.05 (q, J=8.2 Hz, 1H), 3.99 (dd, J=7.8, 3.7 Hz, 1H), 3.87-3.82 (m, 1H), 3.75-3.69 (m, 1H), 3.30-3.16 (m, 2H), 3.02 (dd, J=14.3, 3.8 Hz, 1H), 2.79-2.73 (m, 1H), 2.63-2.54 (m, 2H), 2.19 (br, 1H), 1.78-1.66 (m, 2H).

### Example 70: Synthesis of tert-butyl (2S)-2-{[(1S)-1-cyano-2-[4′-cyano-3′-(trifluoromethyl)-[1,1′-biphenyl]-4-yl]ethyl]carbamoyl}-1,4-oxazepane-4-carboxylate (Compound 7)

### Step 1. Synthesis of tert-butyl (2S)-2-{[(1S)-1-cyano-2-[4′-cyano-3′-(trifluoromethyl)-[1,1′-biphenyl]-4-yl]ethyl]carbamoyl}-1,4-oxazepane-4-carboxylate

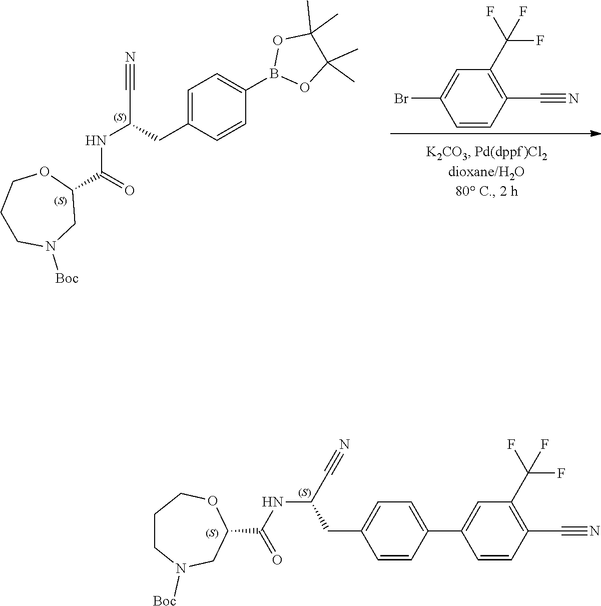

To a solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (100 mg, 0.20 mmol, 1 equiv) and 4-bromo-2-(trifluoromethyl)benzonitrile (50 mg, 0.20 mmol, 1 equiv) in dioxane (5 mL) and H₂O (0.5 mL) were added K₂CO₃ (55 mg, 0.40 mmol, 2 equiv) and Pd(dppf)Cl₂CH₂Cl₂ (16 mg, 0.02 mmol, 0.1 equiv). After stirring for 2 h at 80° C. under a nitrogen atmosphere, the resulting mixture was concentrated under reduced pressure. The residue was purified by silica gel column chromatography, eluted with PE/THF (2:1) to afford tert-butyl (2S)-2-{[(1S)-1-cyano-2-[4′-cyano-3′-(trifluoromethyl)-[1,1′-biphenyl]-4-yl]ethyl]carbamoyl}-1,4-oxazepane-4-carboxylate (60 mg, 55.23%) as a light yellow oil. LCMS (ES, m/z): [M+H]⁺: 543.

### Step 2. Synthesis of (2S)—N-[(1S)-1-cyano-2-[4′-cyano-3′-(trifluoromethyl)-[1,1′-biphenyl]-4-yl]ethyl]-1,4-oxazepane-2-carboxamide

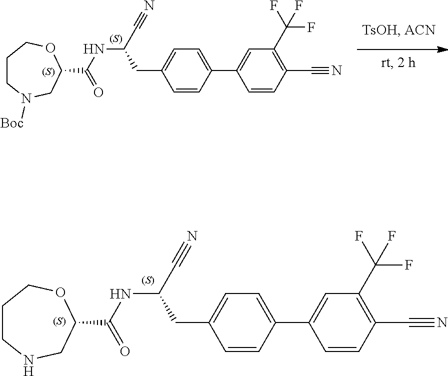

Into a 50 mL round-bottom flask were added tert-butyl (2S)-2-{[(1S)-1-cyano-2-[4′-cyano-3′-(trifluoromethyl)-[1,1′-biphenyl]-4-yl]ethyl]carbamoyl}-1,4-oxazepane-4-carboxylate (60 mg, 0.11 mmol, 1 equiv), ACN (2 mL) and TsOH (57 mg, 0.33 mmol, 3 equiv) at room temperature. The resulting mixture was stirred for 2 h at room temperature. The crude product was purified by Prep-HPLC with the following conditions (Prep-HPLC-013): Column, Atlantis Prep T3 OBD Column, 19*150 mm Sum; mobile phase, Water (0.05% NH₃·H₂O) and ACN (25% PhaseB up to 50% in 10 min) to afford Compound 7 (15.1 mg, 30.86%) as a white solid. LCMS (ES, m/z): [M+H]⁺: 443. ¹H NMR (400 MHz, DMSO-d₆) δ 8.62 (d, J=8.6 Hz, 1H), 8.27-8.20 (m, 3H), 7.84 (d, J=7.9 Hz, 2H), 7.47 (d, J=7.9 Hz, 2H), 5.06 (q, J=8.2 Hz, 1H), 3.99 (dd, J=7.8, 3.7 Hz, 1H), 3.85 (dt, J=11.0, 5.1 Hz, 1H), 3.72 (ddd, J=12.0, 7.6, 4.0 Hz, 1H), 3.28-3.22 (m, 2H), 3.02 (dd, J=14.2, 3.7 Hz, 1H), 2.76 (dt, J=12.3, 5.6 Hz, 1H), 2.64-2.52 (m, 2H), 1.85-1.62 (s, 2H).

### Example 71: Synthesis of (2S)—N-[(1S)-1-cyano-2-[4-(1-oxo-3,4-dihydro-2H-isoquinolin-6-yl)phenyl]ethyl]-1,4-oxazepane-2-carboxamide (Compound 8)

### Step 1. Synthesis of 4-bromo-2-cyclopropylbenzonitrile

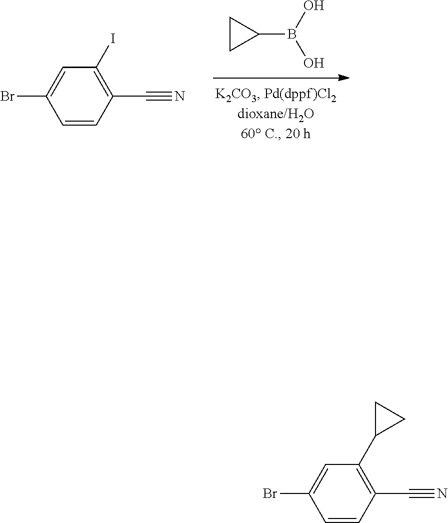 [SMILES: N#Cc1ccc(Br)cc1I ; OB(O)C1CC1 ; N#Cc1ccc(Br)cc1C1CC1]

To a solution of 4-bromo-2-iodobenzonitrile (500 mg, 1.624 mmol, 1 equiv) and cyclopropylboronic acid (278.97 mg, 3.248 mmol, 2 equiv) in dioxane (10 mL) and H₂O (1 mL) were added K₂CO₃ (673.26 mg, 4.872 mmol, 3 equiv) and Pd(dppf)Cl₂CH₂Cl₂ (132.28 mg, 0.162 mmol, 0.1 equiv). After stirring for 2 h at 80° C. under a nitrogen atmosphere, the resulting mixture was concentrated under reduced pressure. The residue was purified by silica gel column chromatography, eluted with PE/THF (2:1) to afford 4-bromo-2-cyclopropylbenzonitrile (250 mg, 55.46%) as a light-yellow oil. NO MS single in LCMS. ¹H NMR (400 MHz, DMSO-d₆) δ 7.71 (d, J=8.2 Hz, 1H), 7.56 (dd, J=8.4, 1.9 Hz, 1H), 7.32 (d, J=1.9 Hz, 1H), 2.15 (tq, J=11.6, 6.4, 5.7 Hz, 1H), 1.20-1.10 (m, 2H), 0.95 (s, OH), 0.95-0.80 (m, 2H).

### Step 2. Synthesis of tert-butyl (2S)-2-{[(1S)-1-cyano-2-{4′-cyano-3′-cyclopropyl-[1,1′-biphenyl]-4-yl}ethyl]carbamoyl}-1,4-oxazepane-4-carboxylate

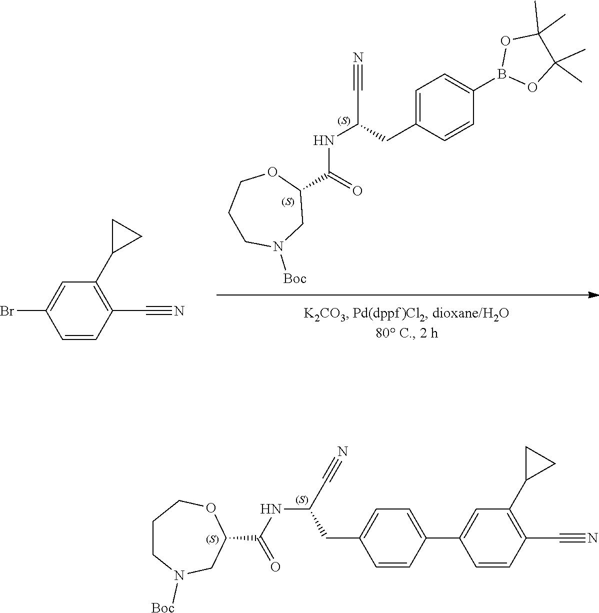

To a solution of 4-bromo-2-cyclopropylbenzonitrile (86.71 mg, 0.312 mmol, 1.2 equiv, 80%) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (130 mg, 0.260 mmol, 1.00 equiv) in dioxane (5 mL) and H₂O (0.5 mL) were added K₂CO₃ (71.95 mg, 0.520 mmol, 2 equiv) and Pd(dppf)Cl₂CH₂Cl₂ (21.20 mg, 0.026 mmol, 0.1 equiv). After stirring for 2 h at 80° C. under a nitrogen atmosphere, the resulting mixture was concentrated under reduced pressure. The residue was purified by silica gel column chromatography, eluted with PE/THF (2:1) to afford tert-butyl (2S)-2-{[(1S)-1-cyano-2-{4′-cyano-3′-cyclopropyl-[1,1′-biphenyl]-4-yl}ethyl]carbamoyl}-1,4-oxazepane-4-carboxylate (80 mg, 59.72%) as a light yellow oil. LCMS (ES, m/z): [M+H]⁺: 515.

### Step 3. Synthesis of (2S)—N-[(1S)-1-cyano-2-{4′-cyano-3′-cyclopropyl-[1,1′-biphenyl]-4-yl}ethyl]-1,4-oxazepane-2-carboxamide

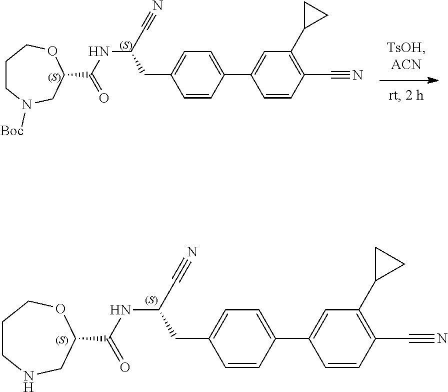

Into a 50 mL round-bottom flask were added tert-butyl (2S)-2-{[(1S)-1-cyano-2-{4′-cyano-3′-cyclopropyl-[1,1′-biphenyl]-4-yl}ethyl]carbamoyl}-1,4-oxazepane-4-carboxylate (80 mg, 0.155 mmol, 1 equiv), ACN (3 mL) and TsOH·H₂O (88.71 mg, 0.465 mmol, 3 equiv) at room temperature. The resulting mixture was stirred for 2 h at room temperature. The crude product was purified by Prep-HPLC with the following conditions (Prep-HPLC-013): Column, Atlantis Prep T3 OBD Column, 19*150 mm Sum; mobile phase, Water (0.05% NH₃·H₂O) and ACN (25% PhaseB up to 50% in 10 min) to afford Compound 8 (16.6 mg, 25.76%) as a white solid. LCMS (ES, m/z): [M+H]⁺: 415. ¹H NMR (400 MHz, DMSO-d₆) δ 8.60 (d, J=8.6 Hz, 1H), 7.82 (d, J=8.1 Hz, 1H), 7.70 (d, J=8.0 Hz, 2H), 7.62 (dd, J=8.1, 1.8 Hz, 1H), 7.41 (d, J=8.0 Hz, 2H), 7.28 (d, J=1.7 Hz, 1H), 5.04 (q, J=8.3 Hz, 1H), 3.99 (dd, J=7.9, 3.7 Hz, 1H), 3.89-3.79 (m, 1H), 3.72 (ddd, J=12.0, 7.7, 4.0 Hz, 1H), 3.28-3.14 (m, 2H), 3.02 (dd, J=14.3, 3.8 Hz, 1H), 2.81-2.71 (m, 1H), 2.65-2.52 (m, 2H), 2.28-2.17 (m, 1H), 1.78-1.63 (m, 2H), 1.19-1.10 (m, 2H), 1.03-0.95 (m, 2H).

### Example 72: Synthesis of (2S)—N-[(1S)-1-cyano-2-{4′-[(4-methylpiperazin-1-yl)methyl]-[1,1′-biphenyl]-4-yl}ethyl]-1,4-oxazepane-2-carboxamide (Compound 9)

### Step 1. Synthesis of tert-butyl (2S)-2-{[(1S)-1-cyano-2-{4′-[(4-methylpiperazin-1-yl)methyl]-[1,1′-biphenyl]-4-yl}ethyl]carbamoyl}-1,4-oxazepane-4-carboxylate

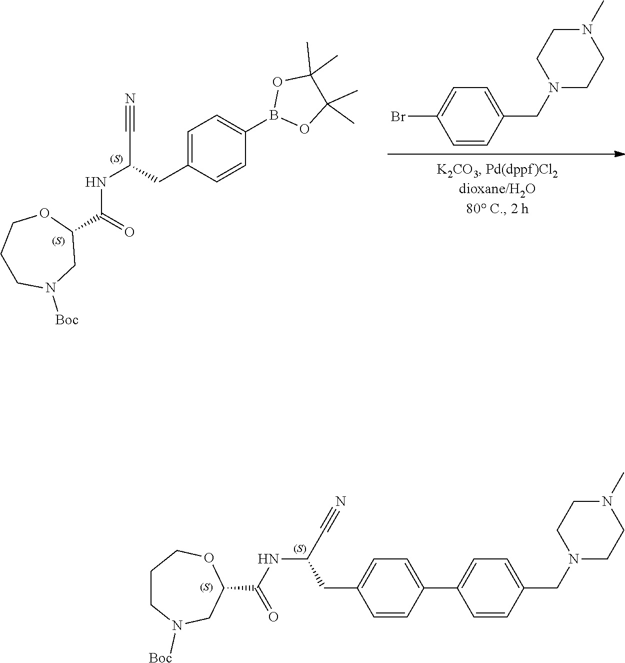

To a solution of 1-[(4-bromophenyl)methyl]-4-methylpiperazine (120 mg, 0.44 mmol, 1.0 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (223 mg, 0.44 mmol, 1.0 equiv) in 1,4-dioxane (5 mL) and H₂O (0.5 mL), K₂CO₃ (123 mg, 0.89 mmol, 2.0 equiv) and Pd(dppf)Cl₂ (33 mg, 0.04 mmol, 0.1 equiv) were added in sequence. The mixture was heated to 80° C. and stirred for 2 h under nitrogen gas atmosphere. The reaction was cooled to room temperature, concentrated under reduced pressure. The residue was purified by silica gel column chromatography, eluted with PE/THF (1:1) to afford tert-butyl (2S)-2-{[(1S)-1-cyano-2-{4′-[(4-methylpiperazin-1-yl)methyl]-[1,1′-biphenyl]-4-yl}ethyl]carbamoyl}-1,4-oxazepane-4-carboxylate (100 mg, 40%) as yellow oil. LCMS (ES, m/z): [M+H]⁺: 562.

### Step 2. Synthesis of (2S)—N-[(1S)-1-cyano-2-{4′-[(4-methylpiperazin-1-yl)methyl]-[1,1′-biphenyl]-4-yl}ethyl]-1,4-oxazepane-2-carboxamide

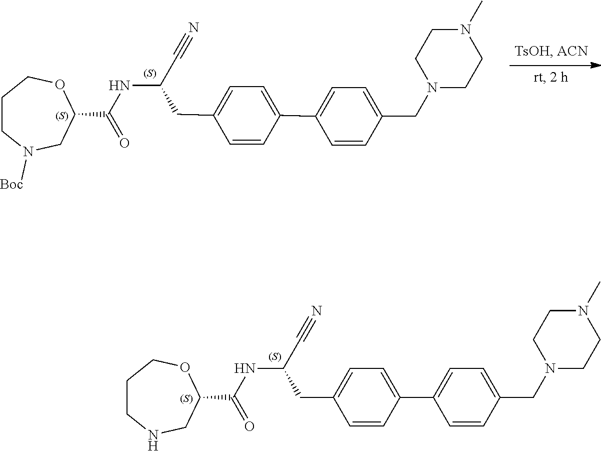

Into a 25 mL round-bottom flask were added tert-butyl (2S)-2-{[(1S)-1-cyano-2-{4′-[(4-methylpiperazin-1-yl)methyl]-[1,1′-biphenyl]-4-yl}ethyl]carbamoyl}-1,4-oxazepane-4-carboxylate (100 mg, 0.17 mmol, 1.0 equiv), TsOH (92 mg, 0.53 mmol, 3.0 equiv) and ACN (3 mL) at room temperature. The resulting mixture was stirred for 2 h at room temperature. The reaction solution was purified by reversed-phase flash chromatography with the following conditions: column, C18-120 g, mobile phase, MeCN in Water (0.1% NH₃·H₂O), 10% to 80% gradient in 10 min; detector, UV 254 nm. This resulted in Compound 9 (23.1 mg, 28%) as yellow oil. LCMS (ES, m/z): [M+H]⁺: 462.3. ¹H NMR (300 MHz, DMSO-d₆) δ 8.60 (d, J=8.5 Hz, 1H), 7.61 (dd, J=8.3, 2.3 Hz, 4H), 7.37 (d, J=7.9 Hz, 4H), 5.02 (q, J=8.2 Hz, 1H), 3.99 (dd, J=7.9, 3.7 Hz, 1H), 3.92-3.65 (m, 2H), 3.48 (s, 2H), 3.24-3.15 (m, 2H), 3.03 (dd, J=14.2, 3.7 Hz, 1H), 2.80-2.71 (m, 1H), 2.65-2.55 (m, 2H), 2.42-2.36 (m, 8H), 2.15 (s, 3H), 1.79-1.65 (m, 2H).

### Example 73: Synthesis of (2S)—N-[(1S)-1-cyano-2-[4-(5-cyano-4-methyl-1,3-thiazol-2-yl)phenyl]ethyl]-1,4-oxazepane-2-carboxamide (Compound 10)

### Step 1. Synthesis of 2-bromo-4-methyl-1,3-thiazole-5-carboxamide

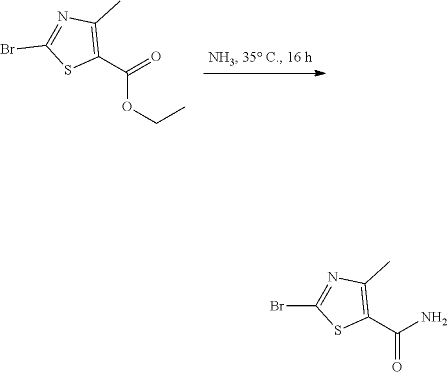

To a stirred solution of ethyl 2-bromo-4-methyl-1,3-thiazole-5-carboxylate (1.8 g, 7.197 mmol, 1.0 equiv) in NH₃·H₂O (50 mL, 30%). The resulting mixture was stirred for 16 h at 35° C. The mixture was allowed to cool down to room temperature. The resulting mixture was diluted with water (50 mL). The resulting mixture was extracted with EtOAc (3×50 mL). The combined organic layers were washed with brine (30 mL), dried over anhydrous Na₂SO₄. After filtration, the filtrate was concentrated under reduced pressure. This resulted in 2-bromo-4-methyl-1,3-thiazole-5-carboxamide (1.1 g, 69.1%) as a light-yellow solid. LCMS (ES) [M+1]⁺ m/z: 221.

### Step 2. Synthesis of 2-bromo-4-methyl-1,3-thiazole-5-carbonitrile

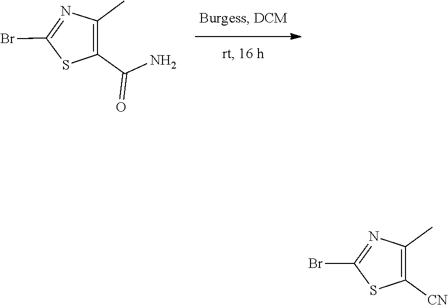

To a stirred solution of 2-bromo-4-methyl-1,3-thiazole-5-carboxamide (0.5 g, 2.262 mmol, 1.0 equiv) in DCM (25 mL) was added Burgess reagent (1.08 g, 4.524 mmol, 2.0 equiv). The resulting mixture was stirred for 16 h at room temperature. The resulting mixture was concentrated under reduced pressure. The residue was purified by silica gel column chromatography, eluted with PE/EA (12:1) to afford 2-bromo-4-methyl-1,3-thiazole-5-carbonitrile (0.4 g, 87.1%) as a white solid. LCMS (ES) [M+1]⁺ m/z: 203.

### Step 3. Synthesis of tert-butyl (2S)-2-{[(1S)-1-cyano-2-[4-(5-cyano-4-methyl-1,3-thiazol-2-yl)phenyl]ethyl]carbamoyl}-1,4-oxazepane-4-carboxylate

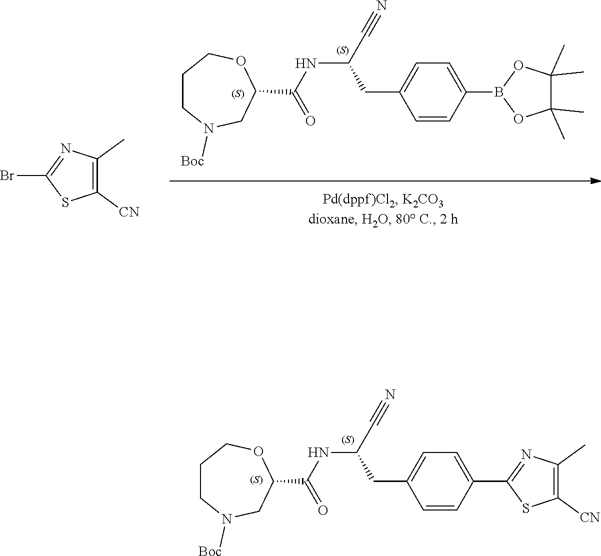

To a stirred solution of 2-bromo-4-methyl-1,3-thiazole-5-carbonitrile (89 mg, 0.440 mmol, 1.1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (200 mg, 0.400 mmol, 1.0 equiv) in dioxane (3 mL) and H₂O (0.3 mL) were added K₂CO₃ (110 mg, 0.800 mmol, 2.0 equiv) and Pd(dppf)Cl₂ (29 mg, 0.040 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (2:1) to afford tert-butyl (2S)-2-{[(1S)-1-cyano-2-[4-(5-cyano-4-methyl-1,3-thiazol-2-yl)phenyl]ethyl]carbamoyl}-1,4-oxazepane-4-carboxylate (100 mg, 50.3%) as a light yellow solid. LCMS (ES) [M+1]⁺ m/z: 496.

### Step 4. Synthesis of (2S)—N-[(1S)-1-cyano-2-[4-(5-cyano-4-methyl-1,3-thiazol-2-yl)phenyl]ethyl]-1,4-oxazepane-2-carboxamide

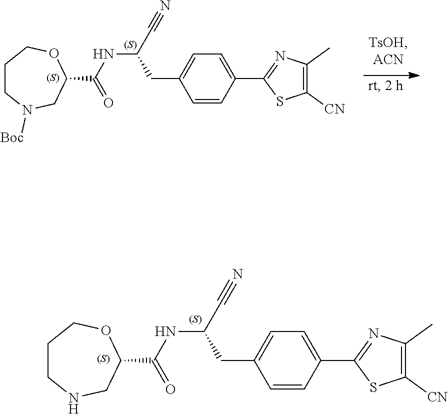

To a stirred solution tert-butyl (2S)-2-{[(1S)-1-cyano-2-[4-(5-cyano-4-methyl-1,3-thiazol-2-yl)phenyl]ethyl]carbamoyl}-1,4-oxazepane-4-carboxylate (100 mg, 0.202 mmol, 1.0 equiv) in ACN (3 mL) was added TsOH (104 mg, 0.606 mmol, 3.0 equiv). The resulting mixture was stirred for 2 h at room temperature. The crude product was purified by Prep-HPLC with the following conditions (Column, XBridge Prep C18 OBD Column, 19*150 mm Sum; mobile phase, Water (10 mmol/L NH₄HCO₃) and ACN (30% PhaseB up to 40% in 7 min); Detector, UV. This resulted in Compound 10 (19.3 mg, 24.1%) as an off-white solid. LCMS (ES) [M+1]⁺ m/z: 396.2. ¹H NMR (300 MHz, DMSO-d₆) δ 8.63 (d, J=8.6 Hz, 1H), 7.95 (d, J=8.3 Hz, 2H), 7.47 (d, J=8.1 Hz, 2H), 5.08 (q, J=8.1 Hz, 1H), 3.98 (dd, J=7.9, 3.6 Hz, 1H), 3.85 (dt, J=12.4, 5.1 Hz, 1H), 3.72 (ddd, J=12.1, 7.4, 4.3 Hz, 1H), 3.25 (d, J=8.0 Hz, 2H), 2.99 (dd, J=14.3, 3.7 Hz, 1H), 2.76 (dt, J=12.1, 5.4 Hz, 1H), 2.63-2.54 (m, 4H), 2.51-2.41 (m, 1H), 2.28 (br, 1H), 1.81-1.60 (m, 2H).

### Example 74: Synthesis of (2S)—N-[(1S)-1-cyano-2-[4-(4-methyl-1,3-thiazol-2-yl)phenyl]ethyl]-1,4-oxazepane-2-carboxamide (Compound 11)

### Step 1. Synthesis of tert-butyl (2S)-2-{[(1S)-1-cyano-2-[4-(4-methyl-1,3-thiazol-2-yl)phenyl]ethyl]carbamoyl}-1,4-oxazepane-4-carboxylate

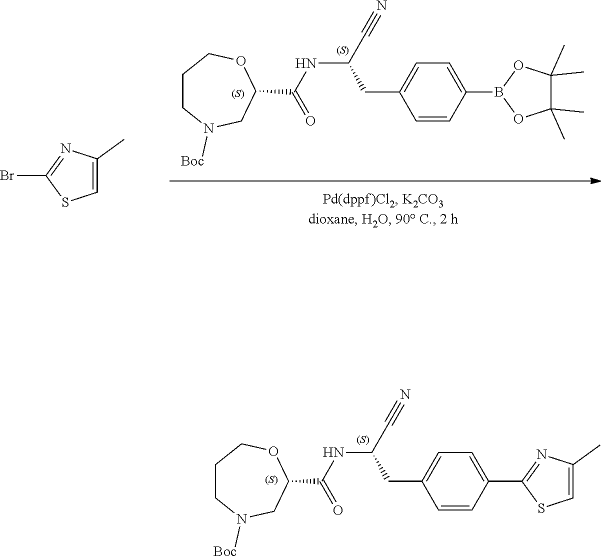

To a stirred solution of 2-bromo-4-methyl-1,3-thiazole (70 mg, 0.39 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (196 mg, 0.39 mmol, 1.0 equiv) in dioxane (1 mL) and H₂O (0.1 mL) were added K₂CO₃ (108 mg, 0.78 mmol, 2.0 equiv) and Pd(dppf)Cl₂ (28 mg, 0.03 mmol, 0.1 equiv). The resulting mixture was stirred for 2 h at 90° C. under nitrogen atmosphere. The mixture was allowed to cool down to room temperature. The resulting mixture was concentrated under reduced pressure. The residue was purified by silica gel column chromatography, eluted with PE/THF (3:1) to afford tert-butyl (2S)-2-{[(1S)-1-cyano-2-[4-(4-methyl-1,3-thiazol-2-yl)phenyl]ethyl]carbamoyl}-1,4-oxazepane-4-carboxylate (130 mg, 70.27%) as an off-white solid. LCMS (ES) [M+1]⁺ m/z: 471.

### Step 2. Synthesis of (2S)—N-[(1S)-1-cyano-2-[4-(4-methyl-1,3-thiazol-2-yl)phenyl]ethyl]-1,4-oxazepane-2-carboxamide

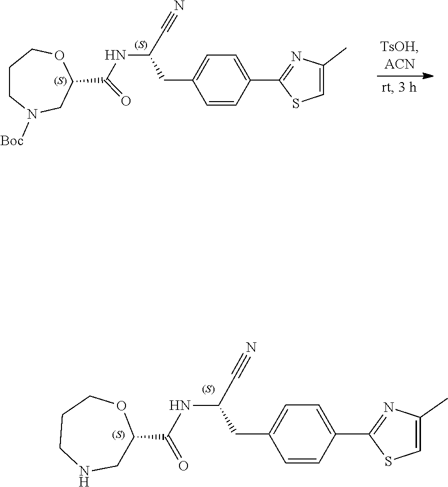

To a stirred solution of tert-butyl (2S)-2-{[(1S)-1-cyano-2-[4-(4-methyl-1,3-thiazol-2-yl)phenyl]ethyl]carbamoyl}-1,4-oxazepane-4-carboxylate (80 mg, 0.17 mmol, 1.0 equiv) in ACN (1 mL) was added TsOH (87 mg, 0.51 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (10 MMOL/L NH₄HCO₃) and ACN (30% PhaseB up to 40% in 7 min); Detector, UV. This resulted in Compound 11 (30 mg, 47.6%) as a white solid. LCMS (ES) [M+1]⁺ m/z: 371.1. ¹H NMR (300 MHz, DMSO-d₆) δ 8.62 (d, J=8.6 Hz, 1H), 7.90-7.81 (m, 2H), 7.40 (d, J=8.2 Hz, 2H), 7.32 (d, J=1.1 Hz, 1H), 5.05 (q, J=8.3 Hz, 1H), 3.98 (dd, J=7.9, 3.6 Hz, 1H), 3.92-3.78 (m, 1H), 3.72 (ddd, J=12.0, 7.3, 4.2 Hz, 1H), 3.21 (d, J=8.1 Hz, 2H), 3.01 (dd, J=14.3, 3.8 Hz, 1H), 2.83-2.69 (m, 1H), 2.66-2.53 (m, 1H), 2.49-2.44 (m, 1H), 2.42 (d, J=1.0 Hz, 3H), 1.72 (q, J=6.2 Hz, 2H).

### Example 75: Synthesis of (2S)—N-[(1S)-1-cyano-2-[4′-(trifluoromethyl)-[1,1′-biphenyl]-4-yl]ethyl]-1,4-oxazepane-2-carboxamide (Compound 12)

### Step 1. Synthesis of tert-butyl (2S)-2-{[(1S)-1-cyano-2-[4′-(trifluoromethyl)-[1,1′-biphenyl]-4-yl]ethyl]carbamoyl}-1,4-oxazepane-4-carboxylate

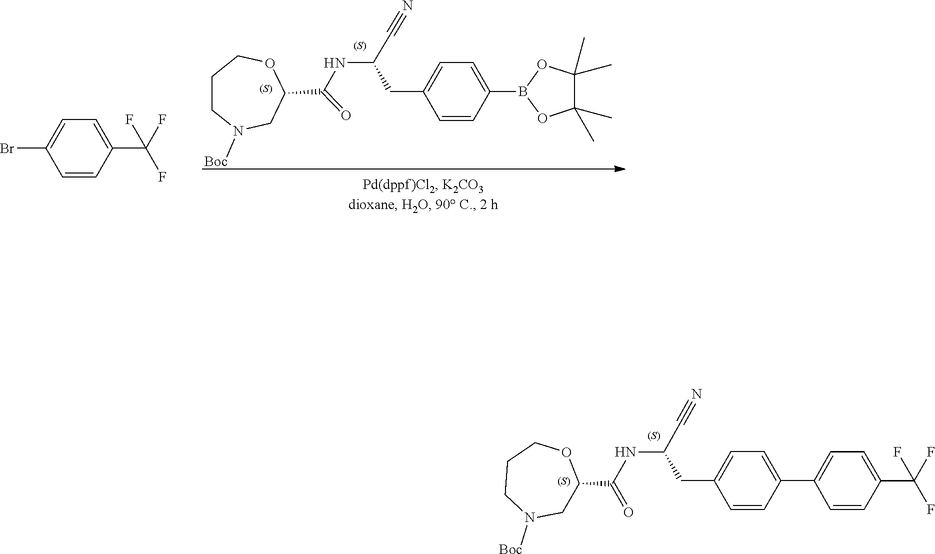

To a stirred solution of 1-bromo-4-(trifluoromethyl)benzene (60 mg, 0.26 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (133 mg, 0.26 mmol, 1.0 equiv) in dioxane (1 mL) and H₂O (0.1 mL) were added K₂CO₃ (73 mg, 0.53 mmol, 2.0 equiv) and Pd(dppf)Cl₂ (19 mg, 0.02 mmol, 0.1 equiv). The resulting mixture was stirred for 2 h at 90° C. under nitrogen atmosphere. The mixture was allowed to cool down to room temperature. The resulting mixture was concentrated under reduced pressure. The residue was purified by silica gel column chromatography, eluted with PE/THF (3:1) to afford tert-butyl (2S)-2-{[(1S)-1-cyano-2-[4′-(trifluoromethyl)-[1,1′-biphenyl]-4-yl]ethyl]carbamoyl}-1,4-oxazepane-4-carboxylate (100 mg, 72.4%) as a light yellow solid. LCMS (ES) [M+1]⁺ m/z: 518.

### Step 2. Synthesis of (2S)—N-[(1S)-1-cyano-2-[4′-(trifluoromethyl)-[1,1′-biphenyl]-4-yl]ethyl]-1,4-oxazepane-2-carboxamide

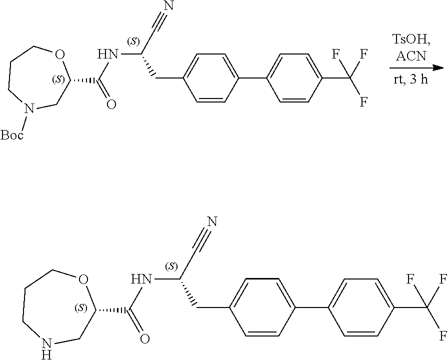

To a stirred solution of tert-butyl (2S)-2-{[(1S)-1-cyano-2-[4′-(trifluoromethyl)-[1,1′-biphenyl]-4-yl]ethyl]carbamoyl}-1,4-oxazepane-4-carboxylate (80 mg, 0.15 mmol, 1.0 equiv) in ACN (1 mL) was added TsOH (79 mg, 0.46 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (10 MMOL/L NH₄HCO₃) and ACN (30% PhaseB up to 40% in 7 min); Detector, UV. This resulted in Compound 12 (20 mg, 31.0%) as an off-white solid. LCMS (ES) [M+1]⁺ m/z: 418.2. ¹H NMR (300 MHz, DMSO-d₆) δ 8.62 (d, J=8.5 Hz, 1H), 7.89 (d, J=8.3 Hz, 2H), 7.81 (d, J=8.3 Hz, 2H), 7.71 (d, J=8.1 Hz, 2H), 7.43 (d, J=8.1 Hz, 2H), 5.05 (q, J=8.2 Hz, 1H), 3.99 (dd, J=7.8, 3.7 Hz, 1H), 3.84 (dt, J=10.7, 5.1 Hz, 1H), 3.72 (ddd, J=12.0, 7.3, 4.2 Hz, 1H), 3.27-3.13 (m, 2H), 3.02 (dd, J=14.3, 3.7 Hz, 1H), 2.76 (dt, J=12.0, 5.5 Hz, 1H), 2.67-2.50 (m, 2H), 1.79-1.62 (m, 2H).

### Example 76: Synthesis of (2S)—N-[(1S)-1-cyano-2-(4-{1′-methyl-2-oxo-1H-spiro[indole-3,4′-piperidin]-5-yl}phenyl) ethyl]-1,4-oxazepane-2-carboxamide (Compound 13)

### Step 1. Synthesis of tert-butyl (2S)-2-{[(1S)-1-cyano-2-(4-{1′-methyl-2-oxo-1H-spiro[indole-3,4′-piperidin]-5-yl}phenyl) ethyl]carbamoyl}-1,4-oxazepane-4-carboxylate

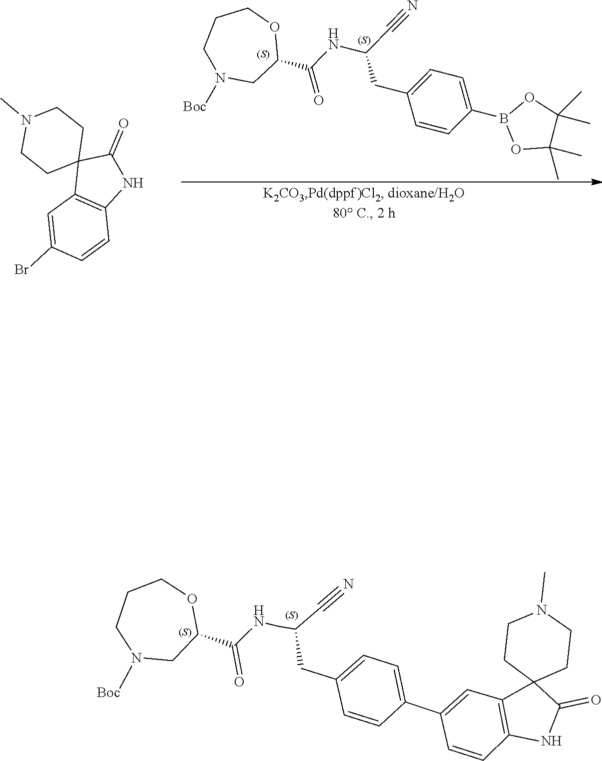

To a stirred solution of 5-bromo-1′-methyl-1H-spiro[indole-3,4′-piperidin]-2-one (120 mg, 0.407 mmol, 1 equiv), tert-butyl (2S)-2-{[(1S)-1-cyano-2-[4-(4,4,5,5-tetramethyl-1,3,2-dioxaborolan-2-yl) phenyl]ethyl]carbamoyl}-1,4-oxazepane-4-carboxylate (223 mg, 0.448 mmol, 1.1 equiv) and K₂CO₃ (112 mg, 0.814 mmol, 2 equiv) in dioxane (1.6 mL) and H₂O (0.2 mL) was added Pd(dppf)Cl₂ (33 mg, 0.041 mmol, 0.1 equiv) at room temperature under nitrogen atmosphere. The resulting mixture was stirred for 2 h at 80° C. under nitrogen atmosphere. The resulting mixture was concentrated under vacuum. The residue was purified by silica gel column chromatography, eluted with PE/EA (1:1) to afford tert-butyl (2S)-2-{[(1S)-1-cyano-2-(4-{1′-methyl-2-oxo-1H-spiro[indole-3,4′-piperidin]-5-yl}phenyl) ethyl]carbamoyl}-1,4-oxazepane-4-carboxylate (100 mg, 41.85%) as a white solid. LCMS (ES) [M+1]m/z: 588.

### Step 2. Synthesis of (2S)—N-[(1S)-1-cyano-2-(4-{1′-methyl-2-oxo-1H-spiro[indole-3,4′-piperidin]-5-yl}phenyl)ethyl]-1,4-oxazepane-2-carboxamide

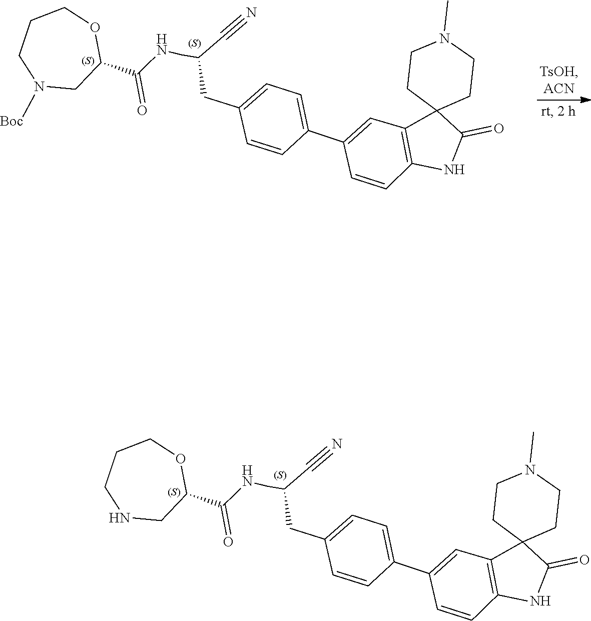

A solution of tert-butyl (2S)-2-{[(1S)-1-cyano-2-(4-{1′-methyl-2-oxo-1H-spiro[indole-3,4′-piperidin]-5-yl}phenyl) ethyl]carbamoyl}-1,4-oxazepane-4-carboxylate (100 mg, 0.170 mmol, 1 equiv) and TsOH (87 mg, 0.510 mmol, 3 equiv) in ACN (2 mL) was stirred for 2 h at room temperature. The residue was purified by reversed-phase flash chromatography with the following conditions: column, C¹⁸ silica gel; mobile phase, MeCN in Water (0.1% NH₃·H₂O), 10% to 60% gradient in 10 min; detector, UV 254 nm. This resulted in Compound 13 (15 mg, 18.08%) as a white solid. LCMS (ES) [M+1]m/z: 488.2. ¹H NMR (400 MHz, DMSO-d6) δ 10.44 (s, 1H), 8.60 (d, J=8.6 Hz, 1H), 7.65 (d, J=1.9 Hz, 1H), 7.61-7.53 (m, 2H), 7.47 (dd, J=8.0, 1.8 Hz, 1H), 7.39-7.32 (m, 2H), 6.92 (d, J=8.1 Hz, 1H), 5.07-4.97 (m, 1H), 4.00 (dd, J=7.8, 3.7 Hz, 1H), 3.84 (ddd, J=12.4, 6.1, 4.4 Hz, 1H), 3.73 (ddd, J=12.1, 7.6, 4.0 Hz, 1H), 3.26-3.12 (m, 2H), 3.03 (dd, J=14.3, 3.8 Hz, 1H), 2.81-2.77 (m, 3H), 2.66-2.55 (m, 4H), 2.31 (s, 3H), 1.90-1.61 (m, 6H).

### Example 77: Synthesis of (2S)—N-[(1S)-1-cyano-2-(4-{1′-methyl-3-oxospiro[2-benzofuran-1,4′-piperidin]-6-yl}phenyl)ethyl]-1,4-oxazepane-2-carboxamide (Compound 14)

### Step 1. Synthesis of 6-bromospiro[2-benzofuran-1,4′-piperidin]-3-one; trifluoroacetic acid

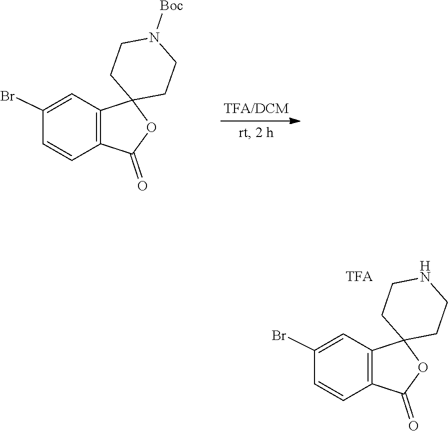 [SMILES: CC(C)(C)OC(=O)N1CCC2(CC1)OC(=O)c1ccc(Br)cc12 ; O=C1OC2(CCNCC2)c2cc(Br)ccc21]

To a stirred solution of tert-butyl 6-bromo-3-oxospiro[2-benzofuran-1,4′-piperidine]-1-carboxylate (300 mg, 0.79 mmol, 1 equiv) and DCM (3 mL) was added TFA (1 mL) dropwise at room temperature. The resulting mixture was stirred for 2 h at room temperature. The resulting mixture was concentrated under reduced pressure. This resulted in 6-bromospiro[2-benzofuran-1,4′-piperidin]-3-one; trifluoroacetic acid (300 mg, 96.49%) as a light yellow oil. LCMS (ES, m/z): [M+H]⁺: 282.

### Step 2. Synthesis of 6-bromo-1′-methylspiro[2-benzofuran-1,4′-piperidin]-3-one

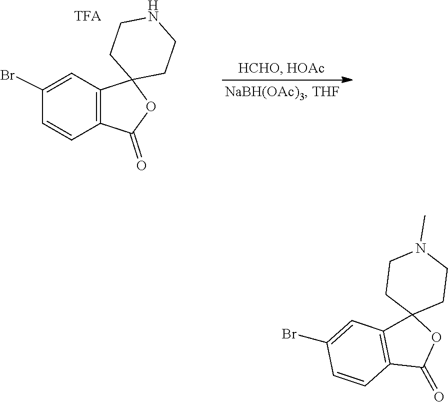 [SMILES: O=C1OC2(CCNCC2)c2cc(Br)ccc21 ; CN1CCC2(CC1)OC(=O)c1ccc(Br)cc12]

To a stirred solution of 6-bromospiro[2-benzofuran-1,4′-piperidin]-3-one; trifluoroacetic acid (300 mg, 0.76 mmol, 1 equiv) and HCHO (598 mg, 7.57 mmol, 10 equiv, 38%) in THF (5 mL) was added HOAc (9 mg, 0.151 mmol, 0.2 equiv) dropwise at room temperature. The resulting mixture was stirred for 1 h at room temperature. To the above mixture was added NaBH(OAc)₃ (321 mg, 1.51 mmol, 2 equiv) at room temperature. The resulting mixture was stirred for additional 1 h at room temperature. The mixture was acidified to pH 8 with saturated NaHCO₃ (aq.). The resulting mixture was extracted with EtOAc (3×30 mL). The combined organic layers were washed with brine (3×50 mL), dried over anhydrous Na₂SO₄. After filtration, the filtrate was concentrated under reduced pressure. This resulted in 6-bromo-1′-methylspiro[2-benzofuran-1,4′-piperidin]-3-one (260 mg, 92.74%) as a light-yellow solid. LCMS (ES, m/z): [M+H]⁺: 296.

### Step 3. Synthesis of tert-butyl (2S)-2-{[(1S)-1-cyano-2-(4-{1′-methyl-3-oxospiro[2-benzofuran-1,4′-piperidin]-6-yl}phenyl)ethyl]carbamoyl}-1,4-oxazepane-4-carboxylate

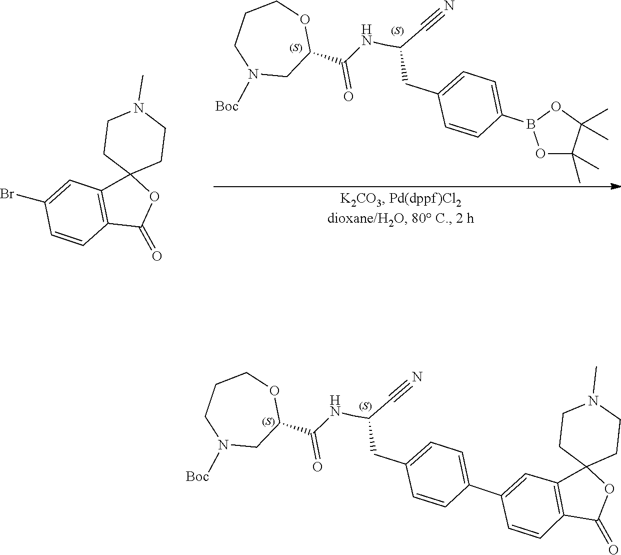

A solution of 6-bromo-1′-methylspiro[2-benzofuran-1,4′-piperidin]-3-one (85 mg, 0.29 mmol, 1.2 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1.00 equiv), K₂CO₃ (66 mg, 0.480 mmol, 2 equiv) and Pd(dppf)Cl₂CH₂Cl₂ (19 mg, 0.02 mmol, 0.1 equiv) in dioxane (5 mL), H₂O (0.5 mL) was stirred for 2 h at 80° C. under nitrogen atmosphere. The residue was purified by silica gel column chromatography, eluted with PE/THF (1:2) to afford tert-butyl (2S)-2-{[(1S)-1-cyano-2-(4-{1′-methyl-3-oxospiro[2-benzofuran-1,4′-piperidin]-6-yl}phenyl)ethyl]carbamoyl}-1,4-oxazepane-4-carboxylate (110 mg, 77.76%) as a white solid. LCMS (ES, m/z): [M+H]⁺: 589.

### Step 4. Synthesis of (2S)—N-[(1S)-1-cyano-2-(4-{1′-methyl-3-oxospiro[2-benzofuran-1,4′-piperidin]-6-yl}phenyl)ethyl]-1,4-oxazepane-2-carboxamide

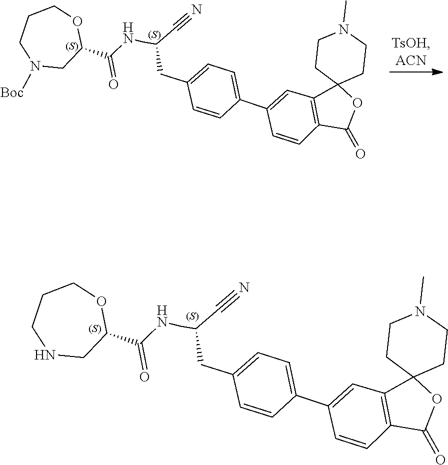

Into a 50 mL round-bottom flask were added tert-butyl (2S)-2-{[(1S)-1-cyano-2-(4-{1′-methyl-3-oxospiro[2-benzofuran-1,4′-piperidin]-6-yl}phenyl)ethyl]carbamoyl}-1,4-oxazepane-4-carboxylate (110 mg, 0.19 mmol, 1 equiv), TsOH·H₂O (107 mg, 0.56 mmol, 3 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 40% gradient in 10 min; detector, UV 254 nm. This resulted in Compound 14 (20.3 mg, 22.24%) as a white solid. LCMS (ES, m/z): [M+H]⁺: 489.3. ¹H NMR (400 MHz, DMSO-d₆) δ 8.62 (d, J=8.5 Hz, 1H), 8.04 (d, J=1.2 Hz, 1H), 7.88 (d, J=1.0 Hz, 2H), 7.78 (d, J=8.3 Hz, 2H), 7.45 (d, J=8.1 Hz, 2H), 5.06 (q, J=8.2 Hz, 1H), 4.00 (dd, J=7.8, 3.7 Hz, 1H), 3.85 (dt, J=10.7, 5.1 Hz, 1H), 3.73 (ddd, J=12.0, 7.3, 4.2 Hz, 1H), 3.28-3.15 (m, 2H), 3.04 (dd, J=14.2, 3.8 Hz, 1H), 2.88-2.72 (m, 3H), 2.68-2.52 (m, 2H), 2.45-2.20 (m, 7H), 1.88-1.52 (m, 4H).

### Example 78: Synthesis of (2S)—N-[(1S)-1-cyano-2-(4-{1,1′-dimethyl-2-oxospiro[indole-3,3′-piperidin]-5-yl}phenyl)ethyl]-1,4-oxazepane-2-carboxamide (Compound 18)

### Step 1. Synthesis of tert-butyl 3-[(2-bromophenyl)carbamoyl]piperidine-1-carboxylate

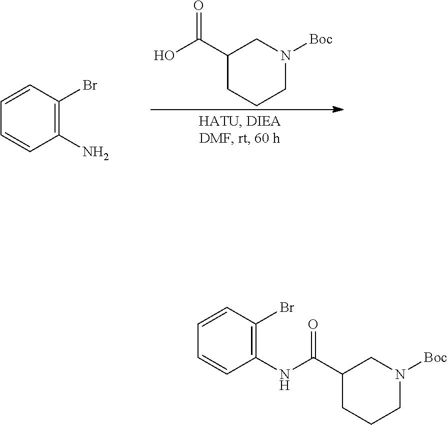 [SMILES: Nc1ccccc1Br ; CC(C)(C)OC(=O)N1CCCC(C(=O)O)C1 ; CC(C)(C)OC(=O)N1CCCC(C(=O)Nc2ccccc2Br)C1]

To a stirred solution of O-bromoaniline (3.71 g, 21.59 mmol, 1.1 equiv) and 1-(tert-butoxycarbonyl)piperidine-3-carboxylic acid (4.5 g, 19.62 mmol, 1.0 equiv) in DCM (40 mL) were added DIEA (5.07 g, 39.25 mmol, 2.0 equiv) and HATU (8.96 g, 23.55 mmol, 1.2 equiv) in portions at 0° C. under nitrogen atmosphere. The resulting mixture was stirred for 60 h at room temperature under nitrogen atmosphere. The resulting mixture was diluted with water (50 mL), extracted with CH₂Cl₂ (3×50 mL). The combined organic layer was washed with brine (50 mL), dried over anhydrous Na₂SO₄. After filtration, the filtrate was concentrated under reduced pressure. The residue was purified by silica gel column chromatography, eluted with PE/THF (9:1) to afford tert-butyl 3-[(2-bromophenyl)carbamoyl]piperidine-1-carboxylate (1.1 g, 14.6%) as a light yellow oil. LCMS (ES) [M+H]⁺ m/z: 383.

### Step 2. Synthesis of tert-butyl 3-[(2-bromophenyl)(methyl)carbamoyl]piperidine-1-carboxylate

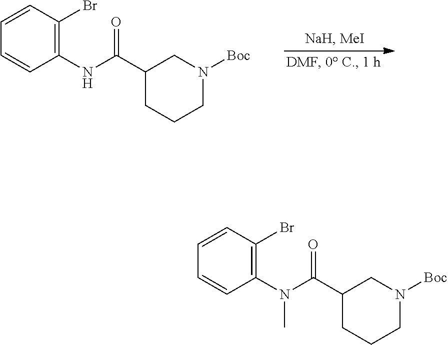 [SMILES: CC(C)(C)OC(=O)N1CCCC(C(=O)Nc2ccccc2Br)C1 ; CN(C(=O)C1CCCN(C(=O)OC(C)(C)C)C1)c1ccccc1Br]

To a stirred solution of tert-butyl 3-[(2-bromophenyl)carbamoyl]piperidine-1-carboxylate (1 g, 2.61 mmol, 1.0 equiv) in DMF (20 mL) were added NaH (156 mg, 3.91 mmol, 1.5 equiv, 60%) in portions at 0° C. under nitrogen atmosphere. The resulting mixture was stirred for 30 min at 0° C. under nitrogen atmosphere. To the above mixture was added MeI (0.56 g, 3.91 mmol, 1.5 equiv) dropwise at 0° C. The resulting mixture was stirred for 1 h at 0° C. under nitrogen atmosphere. The resulting mixture was quenched with water (100 mL), extracted with EtOAc (3×50 mL). The combined organic layer was washed with brine (2×50 mL), dried over anhydrous Na₂SO₄. After filtration, the filtrate was concentrated under reduced pressure. The residue was purified by silica gel column chromatography, eluted with PE/THF (5:1) to afford tert-butyl 3-[(2-bromophenyl)(methyl)carbamoyl]piperidine-1-carboxylate (0.7 g, 67.5%) as a light yellow oil. LCMS (ES) [M+H]⁺ m/z: 397.

### Step 3. Synthesis of tert-butyl 1-methyl-2-oxospiro[indole-3,3′-piperidine]-1′-carboxylate

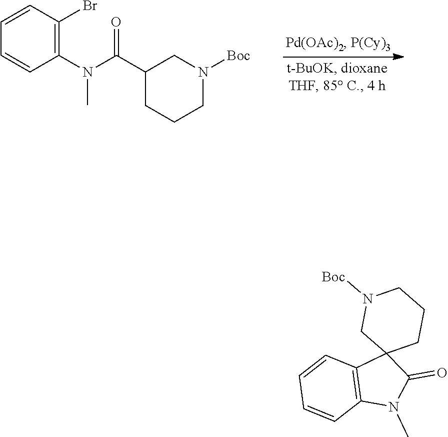 [SMILES: CN(C(=O)C1CCCN(C(=O)OC(C)(C)C)C1)c1ccccc1Br ; CN1C(=O)C2(CCCN(C(=O)OC(C)(C)C)C2)c2ccccc21]

To a stirred solution of tert-butyl 3-[(2-bromophenyl)(methyl)carbamoyl]piperidine-1-carboxylate (600 mg, 1.51 mmol, 1.0 equiv) in dioxane (10 mL) and THF (1 mL) were added t-BuOK (338 mg, 3.02 mmol, 2.0 equiv), Pd(OAc)₂ (33 mg, 0.15 mmol, 0.1 equiv) and PCy₃ (84 mg, 0.30 mmol, 0.2 equiv). The resulting mixture was stirred for 4 h at 85° C. under nitrogen atmosphere. The mixture was allowed to cool down to room temperature, concentrated under reduced pressure to remove the solvent. The residue was purified by silica gel column chromatography, eluted with PE/THF (6:1) to afford tert-butyl 1-methyl-2-oxospiro[indole-3,3′-piperidine]-1′-carboxylate (220 mg, 46%) as a light yellow solid. LCMS (ES) [M+H]⁺ m/z: 317.

### Step 4. Synthesis of tert-butyl 5-bromo-1-methyl-2-oxospiro[indole-3,3′-piperidine]-1′-carboxylate

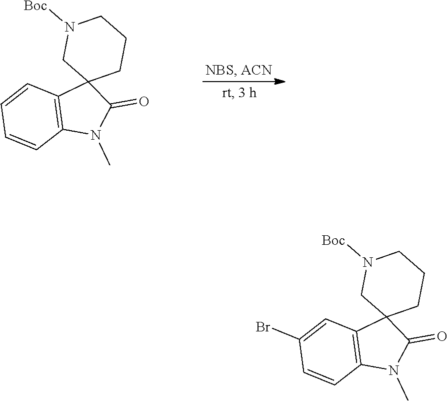 [SMILES: CN1C(=O)C2(CCCN(C(=O)OC(C)(C)C)C2)c2ccccc21 ; CN1C(=O)C2(CCCN(C(=O)OC(C)(C)C)C2)c2cc(Br)ccc21]

To a stirred solution of tert-butyl 1-methyl-2-oxospiro[indole-3,3′-piperidine]-1′-carboxylate (200 mg, 0.63 mmol, 1.0 equiv) in ACN (4 mL) was added NBS (118 mg, 0.66 mmol, 1.05 equiv). The resulting mixture was stirred for 3 h at room temperature. The resulting mixture was diluted with water (20 mL). The resulting mixture was extracted with CH₂Cl₂ (3×20 mL). The combined organic layer was washed with NaHCO₃ (aq.) (20 mL), the combined organic layers were washed with brine (20 mL), dried over anhydrous Na₂SO₄. After filtration, the filtrate was concentrated under reduced pressure. This resulted in tert-butyl 5-bromo-1-methyl-2-oxospiro[indole-3,3′-piperidine]-1′-carboxylate (200 mg, 80%) as a light yellow solid. LCMS (ES) [M+H]⁺ m/z: 395.

### Step 5. Synthesis of 5-bromo-1-methylspiro[indole-3,3′-piperidin]-2-one trifluoroacetic acid

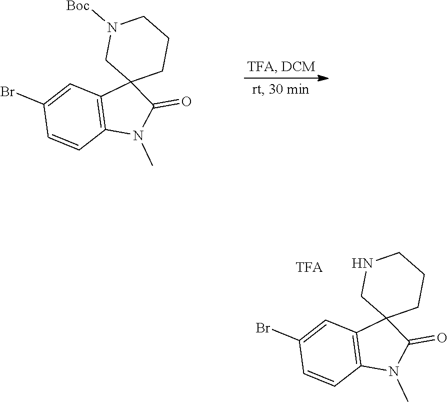 [SMILES: CN1C(=O)C2(CCCN(C(=O)OC(C)(C)C)C2)c2cc(Br)ccc21 ; CN1C(=O)C2(CCCNC2)c2cc(Br)ccc21]

To a stirred solution of tert-butyl 5-bromo-1-methyl-2-oxospiro[indole-3,3′-piperidine]-1′-carboxylate (200 mg, 0.51 mmol, 1.0 equiv) in DCM (3 mL) was added TFA (0.6 mL). The resulting mixture was stirred for 30 min at room temperature. The resulting mixture was concentrated under reduced pressure. This resulted in 5-bromo-1-methylspiro[indole-3,3′-piperidin]-2-one trifluoroacetic acid (120 mg, 80.3%) as a light yellow solid. LCMS (ES) [M-TFA+H]⁺ m/z: 295.

### Step 6. Synthesis of 5-bromo-1,1′-dimethylspiro[indole-3,3′-piperidin]-2-one

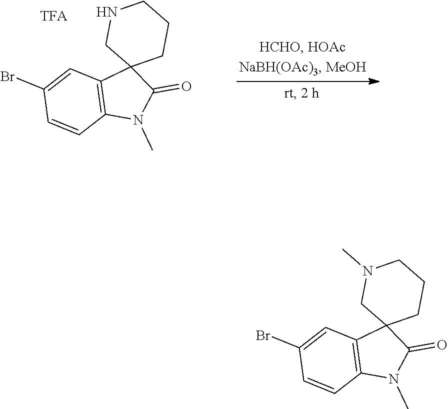 [SMILES: CN1C(=O)C2(CCCNC2)c2cc(Br)ccc21 ; CN1CCCC2(C1)C(=O)N(C)c1ccc(Br)cc12]

To a stirred solution of 5-bromo-1-methylspiro[indole-3,3′-piperidin]-2-one trifluoroacetic acid (100 mg, 0.34 mmol, 1.0 equiv) and HCHO in H₂O (303 mg, 3.39 mmol, 10.0 equiv, 30%) in MeOH (2 mL) were added HOAc (2 mg, 0.03 mmol, 0.1 equiv). The resulting mixture was stirred for 1 h at room temperature. To the above mixture was added NaBH(OAc)₃ (215 mg, 1.02 mmol, 3.0 equiv). The resulting mixture was stirred for 2 h at room temperature. The resulting mixture was diluted with water (20 mL). The residue was basified to pH 8 with saturated NaHCO₃ (aq.). The resulting mixture was extracted with CH₂C12 (3×20 mL). The combined organic layer was washed with brine (20 mL), dried over anhydrous Na₂SO₄. After filtration, the filtrate was concentrated under reduced pressure. This resulted in 5-bromo-1,1′-dimethylspiro[indole-3,3′-piperidin]-2-one (90 mg, 86%) as a light yellow solid. LCMS (ES) [M+H]⁺ m/z: 309.

### Step 7. Synthesis of tert-butyl (2S)-2-{[(1S)-1-cyano-2-(4-{1,1′-dimethyl-2-oxospiro[indole-3,3′-piperidin]-5-yl}phenyl)ethyl]carbamoyl}-1,4-oxazepane-4-carboxylate

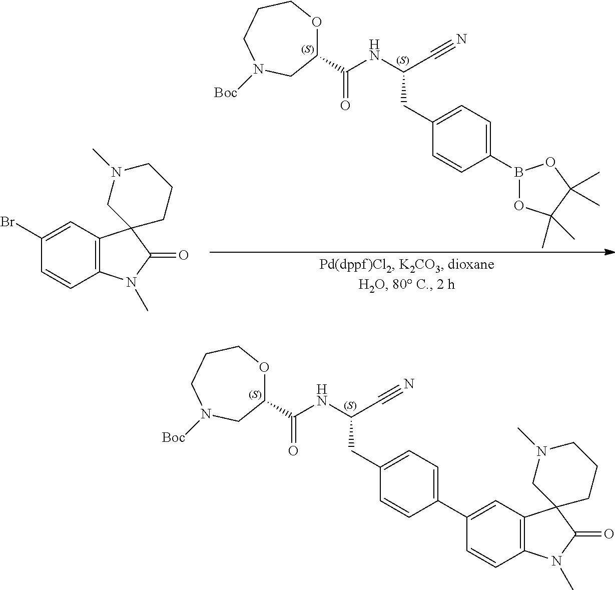

To a stirred solution of 5-bromo-1,1′-dimethylspiro[indole-3,3′-piperidin]-2-one (81 mg, 0.26 mmol, 1.1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1.0 equiv) in dioxane (2 mL) and H₂O (0.2 mL) were added K₂CO₃ (66 mg, 0.48 mmol, 2.0 equiv) and Pd(dppf)Cl₂ (17 mg, 0.02 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (1:1) to afford tert-butyl (2S)-2-{[(1S)-1-cyano-2-(4-{1,1′-dimethyl-2-oxospiro[indole-3,3′-piperidin]-5-yl}phenyl)ethyl]carbamoyl}-1,4-oxazepane-4-carboxylate (100 mg, 69%) as a light yellow oil. LCMS (ES) [M+H]⁺ m/z: 602.

### Step 8. Synthesis of (2S)—N-[(1S)-1-cyano-2-(4-{1,1′-dimethyl-2-oxospiro[indole-3,3′-piperidin]-5-yl}phenyl)ethyl]-1,4-oxazepane-2-carboxamide

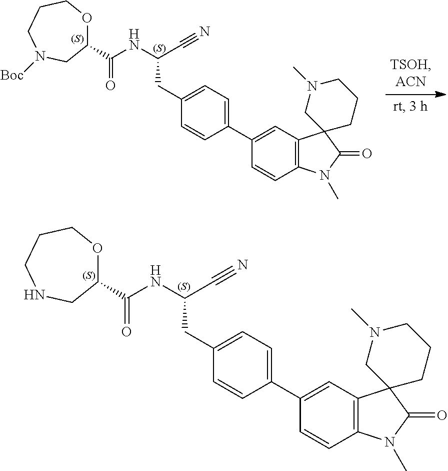

To a stirred solution of tert-butyl (2S)-2-{[(1S)-1-cyano-2-(4-{1,1′-dimethyl-2-oxospiro[indole-3,3′-piperidin]-5-yl}phenyl)ethyl]carbamoyl}-1,4-oxazepane-4-carboxylate (100 mg, 0.17 mmol, 1.0 equiv) in ACN (3 mL) was added TsOH (85 mg, 0.50 mmol, 3.0 equiv). The resulting mixture was stirred for 3 h at room temperature. The reaction solution was purified by Prep-HPLC with the following conditions (Column, XBridge Prep C18 OBD Column, 19*150 mm, 5 um; mobile phase, Water (0.1% NH₃. H₂O) and ACN (10% Phase B up to 80% in 20 min); Detector, UV 254 nm. This resulted in Compound 18 (30 mg, 35.9%) as a white solid. LCMS (ES) [M+1]⁺ m/z: 502.3. ¹H NMR (300 MHz, DMSO-d₆) δ 8.60 (dd, J=8.5, 4.0 Hz, 1H), 7.94 (d, J=1.8 Hz, 1H), 7.59-7.52 (m, 3H), 7.38 (d, J=7.9 Hz, 2H), 7.12 (d, J=8.1 Hz, 1H), 5.07-4.97 (m, 1H), 4.05-3.95 (m, 1H), 3.92-3.79 (m, 1H), 3.77-3.68 (m, 1H), 3.26-3.12 (m, 5H), 3.04 (dd, J=14.4, 3.7 Hz, 1H), 3.00-2.88 (m, 1H), 2.81-2.72 (m, 1H), 2.66-2.54 (m, 2H), 2.49-2.46 (m, 1H), 2.24 (d, J=11.0 Hz, 1H), 2.17 (s, 3H), 2.06-2.01 (m, 2H), 1.77-1.68 (m, 4H), 1.47 (d, J=12.6 Hz, 1H).

### Example 79: Synthesis of (S)—N—((S)-2-(4′-(azetidin-1-ylsulfonyl)-[1,1′-biphenyl]-4-yl)-1-cyanoethyl)-1,4-oxazepane-2-carboxamide (Compound 19)

### Step 1. Synthesis of 1-((4-bromophenyl) sulfonyl) azetidine

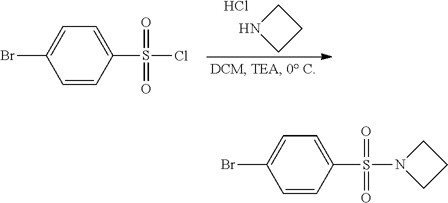

To a stirred solution of azetidine hydrochloride (1.32 g, 14.08 mmol, 1.2 equiv) and TEA (3.56 g, 35.22 mmol, 3 equiv) in DCM (30 mL) was added 4-bromobenzenesulfonyl chloride (3 g, 11.74 mmol, 1 equiv) e in portions at 0° C. The resulting mixture was stirred for 4 h at room temperature. The resulting mixture was diluted with water (40 mL). The resulting mixture was extracted with CH₂Cl₂ (3×20 mL). The combined organic layers were washed with brine (2×30 mL), dried over anhydrous Na₂SO₄. After filtration, the filtrate was concentrated under reduced pressure. This resulted in 1-((4-bromophenyl) sulfonyl) azetidine (2.7 g, 83.27%) as a light yellow solid.

### Step 2. Synthesis of tert-butyl (S)-2-(((S)-2-(4′-(azetidin-1-ylsulfonyl)-[1,1′-biphenyl]-4-yl)-1-cyanoethyl) carbamoyl)-1,4-oxazepane-4-carboxylate

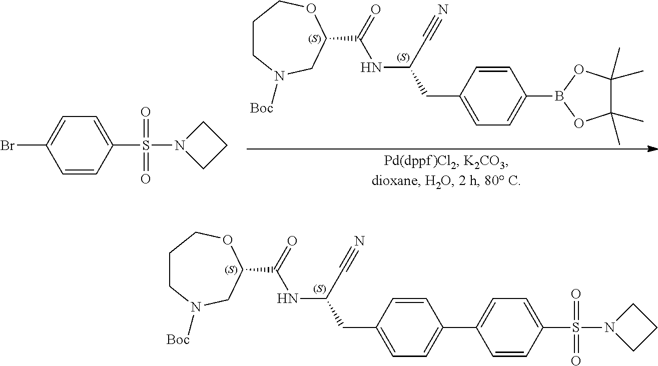

A mixture of 1-((4-bromophenyl) sulfonyl) azetidine (85 mg, 0.30 mmol, 1.2 equiv), tert-butyl (2S)-2-{[(1S)-1-cyano-2-[4-(4,4,5,5-tetramethyl-1,3,2-dioxaborolan-2-yl) phenyl]ethyl]carbamoyl}-1,4-oxazepane-4-carboxylate (128 mg, 0.25 mmol, 1.00 equiv), K₂CO₃ (70 mg, 0.51 mmol, 2 equiv) and Pd(dppf)Cl₂ (20 mg, 0.02 mmol, 0.08 equiv) in dioxane (1.6 mL) and H₂O (0.2 mL) was stirred for 3 h at 80° C. under nitrogen atmosphere. The resulting mixture was concentrated under vacuum. The residue was purified by silica gel column chromatography, eluted with PE/THF (1:1) to afford tert-butyl (S)-2-(((S)-2-(4′-(azetidin-1-ylsulfonyl)-[1,1′-biphenyl]-4-yl)-1-cyanoethyl) carbamoyl)-1,4-oxazepane-4-carboxylate (100 mg, 68.55%) as a white solid. LCMS (ES) [M+1]m/z: 569.

### Step 3. Synthesis of (S)—N—((S)-2-(4′-(azetidin-1-ylsulfonyl)-[1,1′-biphenyl]-4-yl)-1-cyanoethyl)-1,4-oxazepane-2-carboxamide

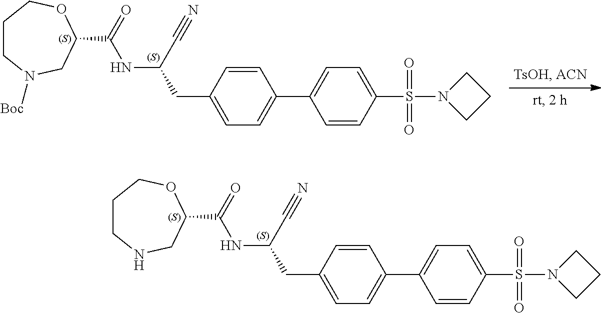

To a stirred solution of tert-butyl (S)-2-(((S)-2-(4′-(azetidin-1-ylsulfonyl)-[1,1′-biphenyl]-4-yl)-1-cyanoethyl) carbamoyl)-1,4-oxazepane-4-carboxylate (110 mg, 0.19 mmol, 1 equiv) in ACN (1.5 mL) was added TsOH (99 mg, 0.57 mmol, 3 equiv) in portions at room temperature. The resulting mixture was stirred for 2 h at room temperature. The residue was purified by reversed-phase flash chromatography with the following conditions: column, C¹⁸ silica gel; mobile phase, MeCN in Water (0.1% NH₃·H₂O), 10% to 70% gradient in 10 min; detector, UV 254 nm. This resulted in Compound 19 (25 mg, 27.58%) as a white solid. ¹H NMR (400 MHz, DMSO-d6) δ 8.62 (d, J=8.7 Hz, 1H), 7.98 (d, J=8.4 Hz, 2H), 7.87 (d, J=8.4 Hz, 2H), 7.76 (d, J=8.1 Hz, 2H), 7.45 (d, J=8.0 Hz, 2H), 5.06 (q, J=8.3 Hz, 1H), 3.99 (dd, J=7.9, 3.7 Hz, 1H), 3.85 (dd, J=11.6, 6.2 Hz, 1H), 3.72 (q, J=7.6 Hz, 5H), 3.27-3.20 (m, 2H), 3.02 (dd, J=14.2, 3.8 Hz, 1H), 2.76 (dd, J=11.9, 6.7 Hz, 1H), 2.64-2.53 (m, 1H), 2.01 (q, J=7.7 Hz, 2H), 1.83-1.62 (m, 2H). LCMS (ES) [M+1]m/z: 469.2.

### Example 80: Synthesis of (2S)—N-[(1S)-1-cyano-2-[4′-(4-methylpiperazin-1-ylsulfonyl)-[1,1′-biphenyl]-4-yl]ethyl]-1,4-oxazepane-2-carboxamide (Compound 20)

### Step 1. Synthesis of 1-(4-bromobenzenesulfonyl)-4-methylpiperazine

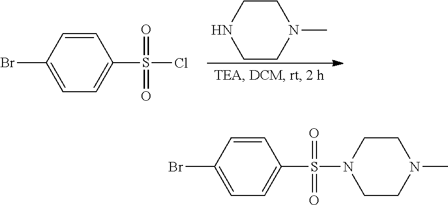

To a solution of methylpiperazine (1.46 g, 14.56 mmol, 1.2 equiv) in DCM (30 mL) was added TEA (3.68 g, 36.40 mmol, 3.0 equiv) at room temperature. This was followed by the addition of 4-bromobenzenesulfonyl chloride (3.1 g, 12.13 mmol, 1.0 equiv) at 0° C. After addition, the mixture was stirred for 2 h at room temperature. The resulting mixture were washed with brine (50 mL), separated out the organic phase, dried over anhydrous Na₂SO₄. After filtration, the filtrate was concentrated under reduced pressure. This resulted in 1-(4-bromobenzenesulfonyl)-4-methylpiperazine (1.6 g, 41%) as a light yellow solid and used to the next step without further purification. LCMS (ES, m/z): [M+H]⁺: 319.

### Step 2. Synthesis of tert-butyl (2S)-2-{[(1S)-1-cyano-2-[4′-(4-methylpiperazin-1-ylsulfonyl)-[1,1′-biphenyl]-4-yl]ethyl]carbamoyl}-1,4-oxazepane-4-carboxylate

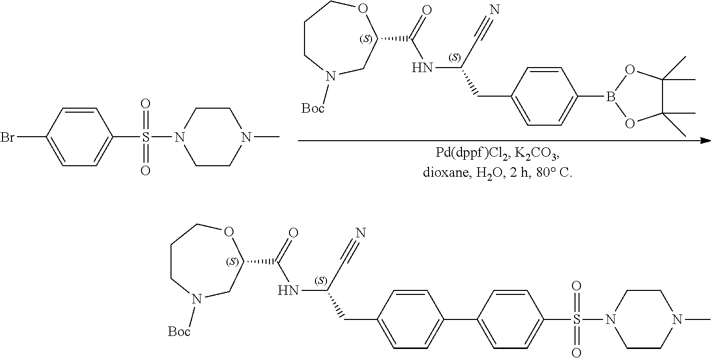

To a solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (146 mg, 0.29 mmol, 1.2 equiv) and 1-(4-bromobenzenesulfonyl)-4-methylpiperazine (78 mg, 0.24 mmol, 1.0 equiv) in dioxane (5 mL) and H₂O (0.5 mL) were added K₂CO₃ (64 mg, 0.46 mmol, 2.0 equiv) and Pd(dppf)Cl₂ (17 mg, 0.02 mmol, 0.1 equiv). After stirring for 2 h at 80° C. under nitrogen atmosphere, the resulting mixture was concentrated under reduced pressure. The residue was purified by silica gel column chromatography, eluted with PE/THF (3:1) to afford tert-butyl (2S)-2-{[(1S)-1-cyano-2-[4′-(4-methylpiperazin-1-ylsulfonyl)-[1,1′-biphenyl]-4-yl]ethyl]carbamoyl}-1,4-oxazepane-4-carboxylate (120 mg, 80%) as a yellow oil. LCMS (ES, m/z): [M+H]⁺: 612.

### Step 3. Synthesis of (2S)—N-[(1S)-1-cyano-2-[4′-(4-methylpiperazin-1-ylsulfonyl)-[1,1′-biphenyl]-4-yl]ethyl]-1,4-oxazepane-2-carboxamide

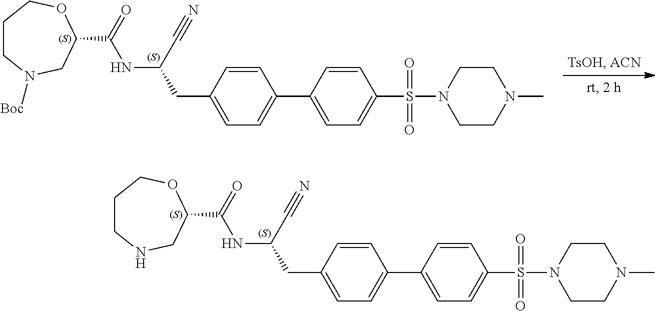

Into a 25 mL round-bottom flask were added tert-butyl (2S)-2-{[(1S)-1-cyano-2-[4′-(4-methylpiperazin-1-ylsulfonyl)-[1,1′-biphenyl]-4-yl]ethyl]carbamoyl}-1,4-oxazepane-4-carboxylate (120 mg, 0.20 mmol, 1.0 equiv), TsOH (101 mg, 0.59 mmol, 3.0 equiv) and ACN (3 mL) at room temperature. The resulting mixture was stirred for 2 h at room temperature. The reaction solution was purified by reversed-phase flash chromatography with the following conditions: C18-120 g column, mobile phase, MeCN in Water (0.05% NH₃·H₂O), 10% to 60% gradient in 10 min; detector, UV 254 nm. The fraction of the target was freezing dried, this resulted in Compound 20 (20.7 mg, 21%) as a white solid. LCMS (ES, m/z): [M+H]⁺: 512.3. ¹H NMR (400 MHz, DMSO-d₆) δ 8.61 (d, J=8.5 Hz, 1H), 7.97-7.90 (m, 2H), 7.80 (d, J=8.3 Hz, 2H), 7.76-7.70 (m, 2H), 7.44 (d, J=8.0 Hz, 2H), 5.06 (q, J=8.2 Hz, 1H), 3.99 (dd, J=7.9, 3.7 Hz, 1H), 3.88-3.82 (m, 1H), 3.76-3.69 (m, 1H), 3.25-3.22 (m, 2H), 3.02 (dd, J=14.3, 3.8 Hz, 1H), 2.96-2.90 (m, 4H), 2.79-2.73 (m, 1H), 2.66-2.55 (m, 2H), 2.39-2.36 (m, 4H), 2.15 (s, 3H), 1.79-1.65 (m, 2H).

### Example 81: Synthesis of (2S)—N-[(1S)-1-cyano-2-[3-fluoro-4′-(4-methylpiperazin-1-ylsulfonyl)-[1,1′-biphenyl]-4-yl]ethyl]-1,4-oxazepane-2-carboxamide (Compound 21)

### Step 1. Synthesis of tert-butyl (2S)-2-{[(1S)-1-cyano-2-[3-fluoro-4′-(4-methylpiperazin-1-ylsulfonyl)-[1,1′-biphenyl]-4-yl]ethyl]carbamoyl}-1,4-oxazepane-4-carboxylate

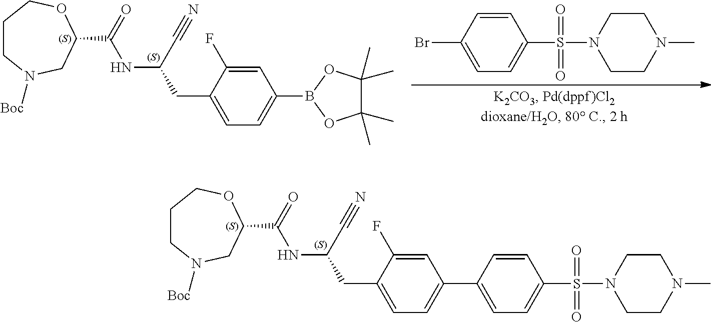

To a stirred mixture of tert-butyl (2S)-2-{[(1S)-1-cyano-2-[2-fluoro-4-(4,4,5,5-tetramethyl-1,3,2-dioxaborolan-2-yl)phenyl]ethyl]carbamoyl}-1,4-oxazepane-4-carboxylate (100 mg, 0.19 mmol, 1.0 equiv), 1-(4-bromobenzenesulfonyl)-4-methylpiperazine (61 mg, 0.19 mmol, 1.0 equiv), K₂CO₃ (53 mg, 0.38 mmol, 2.0 equiv) and Pd(dppf)Cl₂ (14 mg, 0.01 mmol, 0.1 equiv) in dioxane (10 mL) H₂O (1 mL) at room temperature under nitrogen atmosphere. The resulting mixture was stirred for additional 2 h at 80° C. The residue was purified by silica gel column chromatography, eluted with PE/THF (1:1) to afford tert-butyl (2S)-2-{[(1S)-1-cyano-2-[3-fluoro-4′-(4-methylpiperazin-1-ylsulfonyl)-[1,1′-biphenyl]-4-yl]ethyl]carbamoyl}-1,4-oxazepane-4-carboxylate (80 mg, 65.7%) as a yellow oil. LCMS (ES, m/z): [M+H]⁺: 630.

### Step 2. Synthesis of (2S)—N-[(1S)-1-cyano-2-[3-fluoro-4′-(4-methylpiperazin-1-ylsulfonyl)-[1,1′-biphenyl]-4-yl]ethyl]-1,4-oxazepane-2-carboxamide

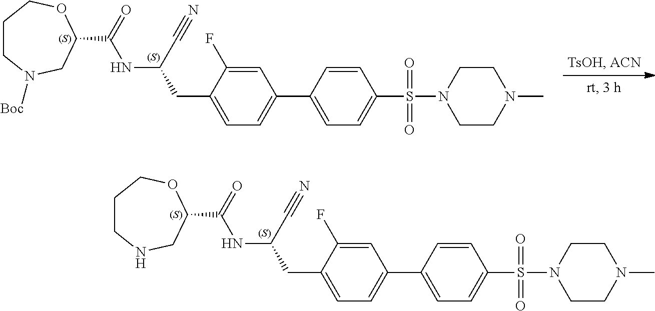

Into a 50 mL round-bottom flask were added tert-butyl (2S)-2-{[(1S)-1-cyano-2-[3-fluoro-4′-(4-methylpiperazin-1-ylsulfonyl)-[1,1′-biphenyl]-4-yl]ethyl]carbamoyl}-1,4-oxazepane-4-carboxylate (80 mg, 0.12 mmol, 1.0 equiv) in ACN (3 mL) and TsOH (66 mg, 0.38 mmol, 3.0 equiv) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 80% gradient in 10 min; detector, UV 254 nm. This resulted in Compound 21 (23.6 mg, 35.08%) as a white solid. LCMS (ES, m/z): [M+H]⁺: 530.3. ¹H NMR (400 MHz, DMSO-d6) δ 8.71 (d, J=8.6 Hz, 1H), 7.99 (d, J=8.5 Hz, 2H), 7.81 (d, J=8.5 Hz, 2H), 7.66 (dd, J=11.3, 1.8 Hz, 1H), 7.62 (dd, J=7.9, 1.9 Hz, 1H), 7.52 (t, J=7.9 Hz, 1H), 5.08 (q, J=8.3 Hz, 1H), 4.00 (dd, J=7.9, 3.7 Hz, 1H), 3.92-3.82 (m, 1H), 3.73 (ddd, J=12.1, 7.8, 4.0 Hz, 1H), 3.23 (dd, J=13.6, 8.8 Hz, 2H), 3.05 (dd, J=14.2, 3.7 Hz, 1H), 2.98-2.89 (m, 4H), 2.83-2.72 (m, 1H), 2.67-2.51 (m, 2H), 2.41-2.34 (m, 4H), 2.15 (s, 3H), 1.83-1.63 (m, 2H).

### Example 82: Synthesis of (2S)—N-[(1S)-1-cyano-2-(4-{2-oxo-3H-spiro[indene-1,4′-piperidin]-6-yl}phenyl)ethyl]-1,4-oxazepane-2-carboxamide (Compound 22)

### Step 1. Synthesis of tert-butyl 5-bromospiro[indene-1,4′-piperidine]-1′-carboxylate) and tert-butyl 6-bromospiro[indene-1,4′-piperidine]-1′-carboxylate

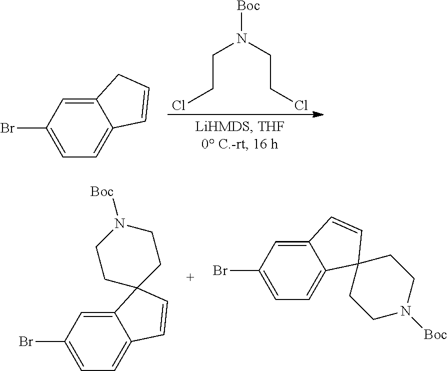

To a stirred solution of 5-bromo-3H-indene (4.0 g, 20.507 mmol, 1.0 equiv) in THF (40 mL) was added LiHMDS (51.27 mL, 51.268 mmol, 2.5 equiv) dropwise at 0° C. under nitrogen atmosphere. The resulting mixture was stirred for 1 h at 0° C. under nitrogen atmosphere. To the above mixture was added tert-butyl N,N-bis(2-chloroethyl)carbamate (5.46 g, 22.558 mmol, 1.1 equiv) dropwise at 0° C. The resulting mixture was stirred for additional 15 h at room temperature. The reaction was quenched by the addition of sat. NH₄Cl (aq.) (20 mL) at 0° C. The resulting mixture was extracted with EtOAc (3×50 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (10:1) to afford mixture of tert-butyl 5-bromospiro[indene-1,4′-piperidine]-1′-carboxylate) and tert-butyl 6-bromospiro[indene-1,4′-piperidine]-1′-carboxylate (4.2 g, 56.22%) as a light-yellow oil. LCMS (ES) [M+H]⁺ m/z: 364.

### Step 2. Synthesis of tert-butyl 6-bromo-2-hydroxy-2,3-dihydrospiro[indene-1,4′-piperidine]-1′-carboxylate) and tert-butyl 6-bromo-3-hydroxy-2,3-dihydrospiro[indene-1,4′-piperidine]-1′-carboxylate) and tert-butyl 5-bromo-3-hydroxy-2,3-dihydrospiro[indene-1,4′-piperidine]-1′-carboxylate) and tert-butyl 5-bromo-2-hydroxy-2,3-dihydrospiro[indene-1,4′-piperidine]-1′-carboxylate

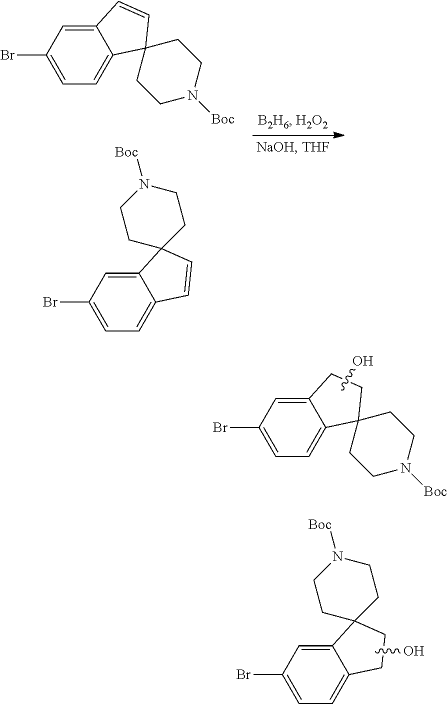

To a stirred mixture of tert-butyl 6-bromospiro[indene-1,4′-piperidine]-1′-carboxylate) and tert-butyl 5-bromospiro[indene-1,4′-piperidine]-1′-carboxylate (4.2 g, 11.529 mmol, 1.0 equiv) in THF (40 mL) was added BH₃-Me₂S (2.63 g, 34.587 mmol, 3.0 equiv) dropwise at 0° C. under nitrogen atmosphere. The resulting mixture was stirred for 16 h at room temperature. To the above mixture was added NaOH (2.31 g, 57.645 mmol, 5.0 equiv) in H₂O (8 mL) and H₂O₂ (6.54 g, 57.645 mmol, 5.0 equiv, 30%) dropwise at 0° C. The resulting mixture was stirred for additional 3 h at room temperature. The reaction was quenched by the addition of Na₂SO₃ (50 mL). The resulting mixture was extracted with EtOAc (3×50 mL). The combined organic layers were washed with brine (100 mL), dried over anhydrous Na2SO4. After filtration, the filtrate was concentrated under reduced pressure. The residue was purified by silica gel column chromatography, eluted with PE/THF (10:1) to afford mixture of tert-butyl 6-bromo-2-hydroxy-2,3-dihydrospiro[indene-1,4′-piperidine]-1′-carboxylate) and tert-butyl 6-bromo-3-hydroxy-2,3-dihydrospiro[indene-1,4′-piperidine]-1′-carboxylate) and tert-butyl 5-bromo-3-hydroxy-2,3-dihydrospiro[indene-1,4′-piperidine]-1′-carboxylate) and tert-butyl 5-bromo-2-hydroxy-2,3-dihydrospiro[indene-1,4′-piperidine]-1′-carboxylate (3.0 g, 68.06%) as a off-white oil. LCMS (ES) [M+H]⁺ m/z: 382.

### Step 3. Synthesis of tert-butyl 5-bromo-2-oxo-3H-spiro[indene-1,4′-piperidine]-1′-carboxylate and tert-butyl 6-bromo-2-oxo-3H-spiro[indene-1,4′-piperidine]-1′-carboxylate and tert-butyl 5-bromo-3-hydroxy-octahydrospiro[indene-1,4′-piperidine]-1′-carboxylate and tert-butyl 6-bromo-3-oxo-2H-spiro[indene-1,4′-piperidine]-1′-carboxylate

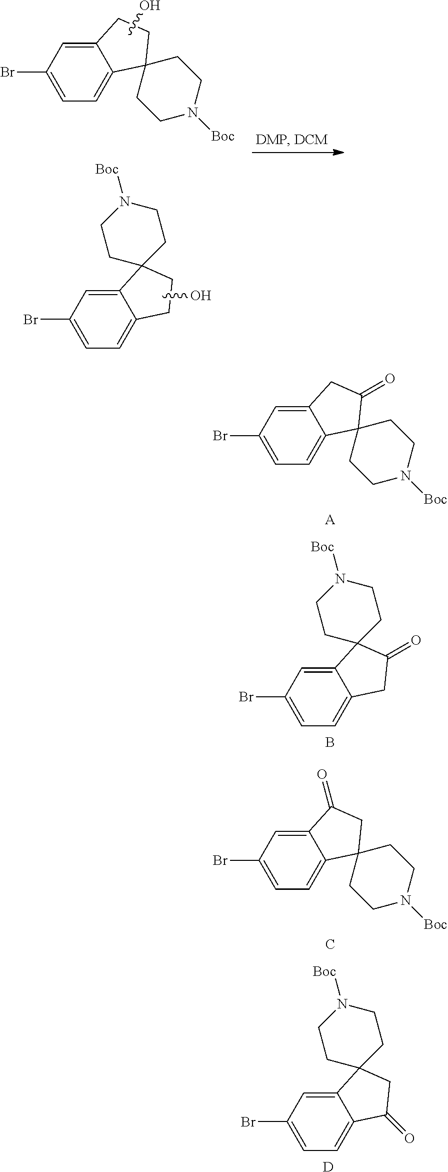

To a stirred mixture of tert-butyl 6-bromo-2-hydroxy-2,3-dihydrospiro[indene-1,4′-piperidine]-1′-carboxylate) and tert-butyl 5-bromo-2-hydroxy-2,3-dihydrospiro[indene-1,4′-piperidine]-1′-carboxylate) and tert-butyl 5-bromo-3-hydroxy-2,3-dihydrospiro[indene-1,4′-piperidine]-1′-carboxylate) and tert-butyl 6-bromo-3-hydroxy-2,3-dihydrospiro[indene-1,4′-piperidine]-1′-carboxylate (3.0 g, 7.847 mmol, 1.0 equiv) in DCM (40 mL) was added DMP (6.66 g, 15.694 mmol, 2.0 equiv) in portions at room temperature. The resulting mixture was stirred for 3 h at room temperature. The resulting mixture was extracted with CH₂Cl₂ (3×40 mL). The combined organic layers were washed with brine (50 mL), dried over anhydrous Na₂SO₄. After filtration, the filtrate was concentrated under reduced pressure. The residue was purified by silica gel column chromatography, eluted with PE/THF (12:1) to afford tert-butyl 5-bromo-2-oxo-3H-spiro[indene-1,4′-piperidine]-1′-carboxylate (620 mg, 20.78%) and tert-butyl 6-bromo-2-oxo-3H-spiro[indene-1,4′-piperidine]-1′-carboxylate (490 mg, 16.42%) and tert-butyl 5-bromo-3-hydroxy-octahydrospiro[indene-1,4′-piperidine]-1′-carboxylate (730 mg, 23.95%) and tert-butyl 6-bromo-3-oxo-2H-spiro[indene-1,4′-piperidine]-1′-carboxylate (580 mg, 19.44%) as a off-white solid. LCMS (ES) [M−H]⁻ m/z: 380.

A: ¹H NMR (400 MHz, DMSO-d₆) δ 7.55 (d, J=1.9 Hz, 1H), 7.47 (dd, J=8.2, 2.0 Hz, 1H), 7.37 (d, J=8.2 Hz, 1H), 3.78 (dd, J=13.5, 4.6 Hz, 2H), 3.68 (s, 2H), 3.34 (s, 2H), 1.80-1.59 (m, 4H), 1.43 (s, 9H).

B: ¹H NMR (400 MHz, DMSO-d₆) δ 7.63 (d, J=1.9 Hz, 1H), 7.47 (dd, J=8.1, 1.9 Hz, 1H), 7.34-7.27 (m, 1H), 3.81 (d, J=13.5 Hz, 2H), 3.62 (s, 2H), 3.33 (s, 2H), 1.77-1.6C9 (m, 4H), 1.44 (s, 9H).

C: ¹H NMR (400 MHz, DMSO-d₆) δ 7.86 (dd, J=8.2, 2.0 Hz, 1H), 7.76-7.67 (m, 2H), 4.02 (d, J=12.5 Hz, 2H), 2.87 (s, 2H), 2.71 (s, 2H), 1.86 (td, J=13.0, 4.6 Hz, 2H), 1.52-1.45 (m, 2H), 1.43 (s, 9H).

D: ¹H NMR (400 MHz, DMSO-d₆) δ 8.05 (d, J=1.6 Hz, 1H), 7.64 (dd, J=8.2, 1.7 Hz, 1H), 7.54 (d, J=8.1 Hz, 1H), 4.02 (s, 2H), 2.86 (s, 2H), 2.68 (s, 2H), 1.91 (td, J=13.1, 12.7, 4.5 Hz, 2H), 1.48 (d, J=12.7 Hz, 2H), 1.44 (s, 9H).

### Step 4. Synthesis of tert-butyl 6-{4-[(2S)-2-{[(2S)-4-(tert-butoxycarbonyl)-1,4-oxazepan-2-yl]formamido}-2-cyanoethyl]phenyl}-2-oxo-3H-spiro[indene-1,4′-piperidine]-1′-carboxylate

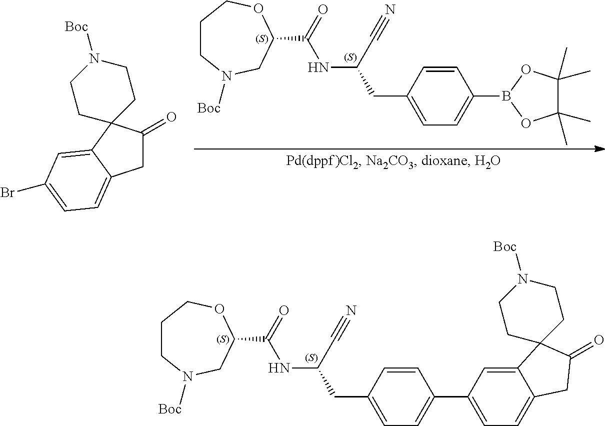

To a stirred solution of tert-butyl 6-bromo-2-oxo-3H-spiro[indene-1,4′-piperidine]-1′-carboxylate (120 mg, 0.316 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (173 mg, 0.348 mmol, 1.1 equiv) in dioxane (2 mL) and H₂O (0.2 mL) were added Na₂CO₃ (66 mg, 0.632 mmol, 2.0 equiv) and Pd(dppf)Cl₂ (23 mg, 0.032 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (2:1) to afford tert-butyl 6-{4-[(2S)-2-{[(2S)-4-(tert-butoxycarbonyl)-1,4-oxazepan-2-yl]formamido}-2-cyanoethyl]phenyl}-2-oxo-3H-spiro[indene-1,4′-piperidine]-1′-carboxylate (140 mg, 65.94%) as a light yellow solid. LCMS (ES) [M+H]⁺ m/z: 673.

### Step 5. Synthesis of (2S)—N-[(1S)-1-cyano-2-(4-{2-oxo-3H-spiro[indene-1,4′-piperidin]-6-yl}phenyl)ethyl]-1,4-oxazepane-2-carboxamide

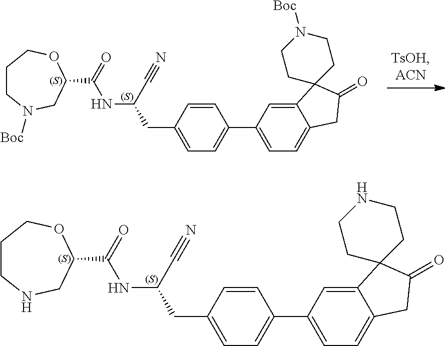

To a stirred solution of tert-butyl 6-{4-[(2S)-2-{[(2S)-4-(tert-butoxycarbonyl)-1,4-oxazepan-2-yl]formamido}-2-cyanoethyl]phenyl}-2-oxo-3H-spiro[indene-1,4′-piperidine]-1′-carboxylate (140 mg, 0.208 mmol, 1.0 equiv) in ACN (2 mL) was added TsOH (107 mg, 0.624 mmol, 3.0 equiv). The resulting mixture was stirred for 3 h at room temperature. The mixture was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in Compound 22 (20 mg, 20.34%) as a light brown solid. LCMS (ES) [M+H]⁺ m/z: 473. ¹H NMR (400 MHz, DMSO-d₆) δ 8.61 (d, J=8.6 Hz, 1H), 7.69-7.50 (m, 4H), 7.44-7.29 (m, 3H), 5.03 (q, J=8.2 Hz, 1H), 4.00 (dd, J=7.9, 3.7 Hz, 1H), 3.90-3.79 (m, 1H), 3.78-3.67 (m, 1H), 3.65 (s, 2H), 3.28-3.13 (m, 3H), 3.10-2.97 (m, 3H), 2.91-2.84 (m, 1H), 2.82-2.71 (m, 1H), 2.66-2.51 (m, 2H), 1.78-1.68 (m, 6H).

### Example 83: Synthesis of (2S)—N-[(1S)-1-cyano-2-(4-{1-methyl-2-oxospiro[indole-3,4′-piperidin]-5-yl}phenyl)ethyl]-1,4-oxazepane-2-carboxamide (Compound 23)

### Step 1. Synthesis of tert-butyl 4-[(2-bromophenyl)carbamoyl]piperidine-1-carboxylate

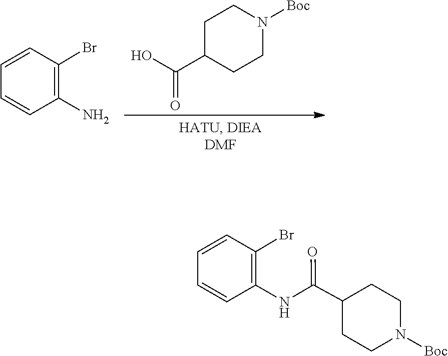 [SMILES: Nc1ccccc1Br ; CC(C)(C)OC(=O)N1CCC(C(=O)O)CC1 ; CC(C)(C)OC(=O)N1CCC(C(=O)Nc2ccccc2Br)CC1]

To a stirred mixture of O-bromoaniline (3.0 g, 17.43 mmol, 1.0 equiv), 1-(tert-butoxycarbonyl)piperidine-4-carboxylic acid (4.0 g, 17.43 mmol, 1.0 equiv) and DIEA (6.8 g, 52.31 mmol, 3.0 equiv) in DMF (30 mL) was added HATU (7.9 g, 20.92 mmol, 1.2 equiv) in portions at 0° C. The resulting mixture was stirred for additional 16 h at room temperature. The resulting mixture was extracted with EtOAc (3×100 mL). The combined organic layers were washed with brine (3×100 mL), dried over anhydrous Na₂SO₄. The residue was purified by silica gel column chromatography, eluted with PE/THF (1:1) to afford tert-butyl 4-[(2-bromophenyl)carbamoyl]piperidine-1-carboxylate (3.9 g, 58.3%) as a yellow oil. LCMS (ES, m/z): [M+H]⁺: 383.

### Step 2. Synthesis of tert-butyl 4-[(2-bromophenyl)(methyl)carbamoyl]piperidine-1-carboxylate

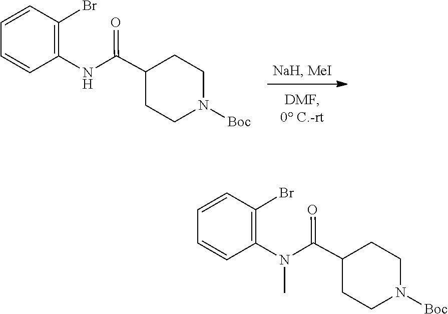 [SMILES: CC(C)(C)OC(=O)N1CCC(C(=O)Nc2ccccc2Br)CC1 ; CN(C(=O)C1CCN(C(=O)OC(C)(C)C)CC1)c1ccccc1Br]

To a stirred mixture of tert-butyl 4-[(2-bromophenyl)carbamoyl]piperidine-1-carboxylate (3.9 g, 10.17 mmol, 1.0 equiv) in DMF (40 mL) was added NaH (0.6 g, 15.26 mmol, 1.5 equiv, 60%), MeI (1.7 g, 12.21 mmol, 1.2 equiv) in portions at 0° C. under nitrogen atmosphere. The resulting mixture was stirred for additional 3 h at 0° C.-room temperature. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/THF (3:1) to afford tert-butyl 4-[(2-bromophenyl)(methyl)carbamoyl]piperidine-1-carboxylate (2.2 g, 54.4%) as a yellow solid. LCMS (ES, m/z): [M+H]⁺: 397.

### Step 3. Synthesis of tert-butyl 1-methyl-2-oxospiro[indole-3,4′-piperidine]-1′-carboxylate

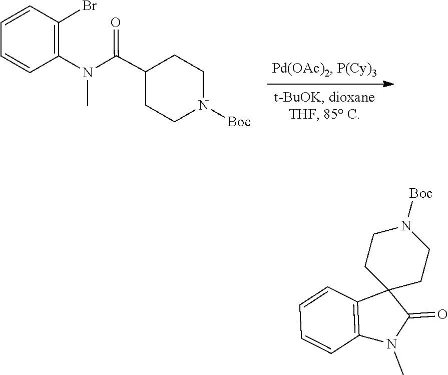 [SMILES: CN(C(=O)C1CCN(C(=O)OC(C)(C)C)CC1)c1ccccc1Br ; CN1C(=O)C2(CCN(C(=O)OC(C)(C)C)CC2)c2ccccc21]

A solution of tert-butyl 4-[(2-bromophenyl)(methyl)carbamoyl]piperidine-1-carboxylate (1.0 g, 2.51 mmol, 1.0 equiv), Pd(OAc)₂ (0.1 g, 0.25 mmol, 0.1 equiv) and t-BuOK (0.6 g, 5.03 mmol, 2.0 equiv) in 1,4-dioxane (10 mL) THF (1 mL) was stirred for 4 h at 85° C. under nitrogen atmosphere. The residue was purified by silica gel column chromatography, eluted with PE/THF (5:1) to afford tert-butyl 1-methyl-2-oxospiro[indole-3,4′-piperidine]-1′-carboxylate (330 mg, 41.44%) as a yellow oil. LCMS (ES, m/z): [M+H]⁺: 313.

### Step 4. Synthesis of tert-butyl 5-bromo-1-methyl-2-oxospiro[indole-3,4′-piperidine]-1′-carboxylate

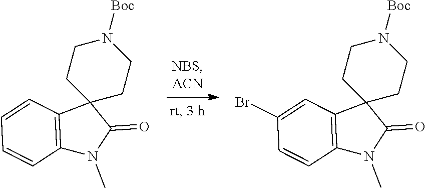 [SMILES: CN1C(=O)C2(CCN(C(=O)OC(C)(C)C)CC2)c2ccccc21 ; CN1C(=O)C2(CCN(C(=O)OC(C)(C)C)CC2)c2cc(Br)ccc21]

Into a 50 mL round-bottom flask were added tert-butyl 1-methyl-2-oxospiro[indole-3,4′-piperidine]-1′-carboxylate (330 mg, 1.04 mmol, 1.0 equiv) in ACN (5 mL) and NBS (194 mg, 1.09 mmol, 1.0 equiv) at room temperature. The resulting mixture was extracted with CH₂Cl₂ (3×30 mL). The combined organic layers were washed with brine (3×30 mL), dried over anhydrous Na₂SO₄. After filtration, the filtrate was concentrated under reduced pressure. This resulted in tert-butyl 5-bromo-1-methyl-2-oxospiro[indole-3,4′-piperidine]-1′-carboxylate (360 mg, 87.3%) as a yellow oil. LCMS (ES, m/z): [M+H]⁺: 395.

### Step 5. Synthesis of tert-butyl 5-{4-[(2S)-2-{[(2S)-4-(tert-butoxycarbonyl)-1,4-oxazepan-2-yl]formamido}-2-cyanoethyl]phenyl}-1-methyl-2-oxospiro[indole-3,4′-piperidine]-1′-carboxylate

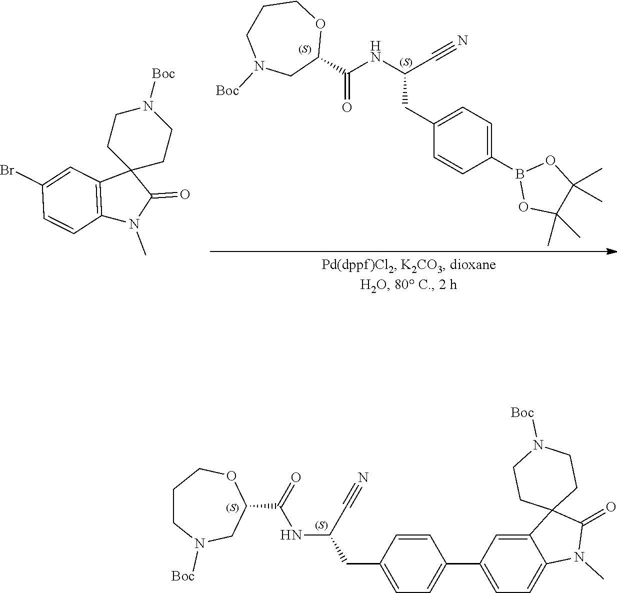

A solution of tert-butyl 5-bromo-1-methyl-2-oxospiro[indole-3,4′-piperidine]-1′-carboxylate (158 mg, 0.40 mmol, 1.0 equiv), tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (200 mg, 0.40 mmol, 1.0 equiv), K₂CO₃ (111 mg, 0.80 mmol, 2.0 equiv) and Pd(dppf)Cl₂ (29 mg, 0.04 mmol, 0.1 equiv) in 1,4-dioxane (10 mL) H₂O (1 mL) was stirred for 3 h at 80° C. under nitrogen atmosphere. The residue was purified by silica gel column chromatography, eluted with PE/THF (2:1) to afford tert-butyl 5-{4-[(2S)-2-{[(2S)-4-(tert-butoxycarbonyl)-1,4-oxazepan-2-yl]formamido}-2-cyanoethyl]phenyl}-1-methyl-2-oxospiro[indole-3,4′-piperidine]-1′-carboxylate (100 mg, 36.30%) as a yellow oil. LCMS (ES, m/z): [M+H]⁺: 688.

### Step 6. Synthesis of (2S)—N-[(1S)-1-cyano-2-(4-{1-methyl-2-oxospiro[indole-3,4′-piperidin]-5-yl}phenyl)ethyl]-1,4-oxazepane-2-carboxamide

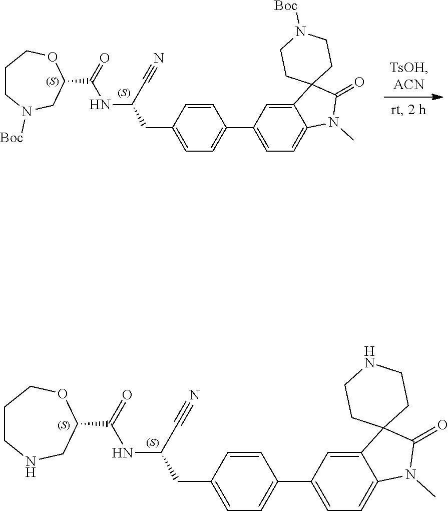

Into a 50 mL round-bottom flask were added tert-butyl 5-{4-[(2S)-2-{[(2S)-4-(tert-butoxycarbonyl)-1,4-oxazepan-2-yl]formamido}-2-cyanoethyl]phenyl}-1-methyl-2-oxospiro[indole-3,4′-piperidine]-1′-carboxylate (100 mg, 0.14 mmol, 1.0 equiv) and TsOH (75 mg, 0.43 mmol, 3.0 equiv) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 80% gradient in 10 min; detector, UV 254 nm. This resulted in Compound 23 (16.3 mg, 22.99%) as a white solid. LCMS (ES, m/z): [M+H]⁺: 488.2. ¹H NMR (400 MHz, DMSO-d6) δ 8.72-8.61 (m, 1H), 7.73 (s, 1H), 7.69-7.47 (m, 3H), 7.45-7.18 (m, 2H), 7.17-7.02 (m, 1H), 5.23-4.71 (m, 1H), 4.05-3.65 (m, 4H), 3.43-2.85 (m, 12H), 2.81-2.54 (m, 2H), 2.01-1.42 (m, 6H).

### Example 84: Synthesis of (2S,3aS,6aR)—N-[(1S)-1-cyano-2-{2-fluoro-4-[2-oxo-3-(2-propoxyethyl)-1,3-benzoxazol-5-yl]phenyl}ethyl]-hexahydro-1H-furo[3,4-b]pyrrole-2-carboxamide (Compound 24)

### Step 1. Synthesis of tert-butyl (2S,3aS,6aR)-2-{[(1S)-1-cyano-2-{2-fluoro-4-[2-oxo-3-(2-propoxyethyl)-1,3-benzoxazol-5-yl]phenyl}ethyl]carbamoyl}-hexahydrofuro[3,4-b]pyrrole-1-carboxylate

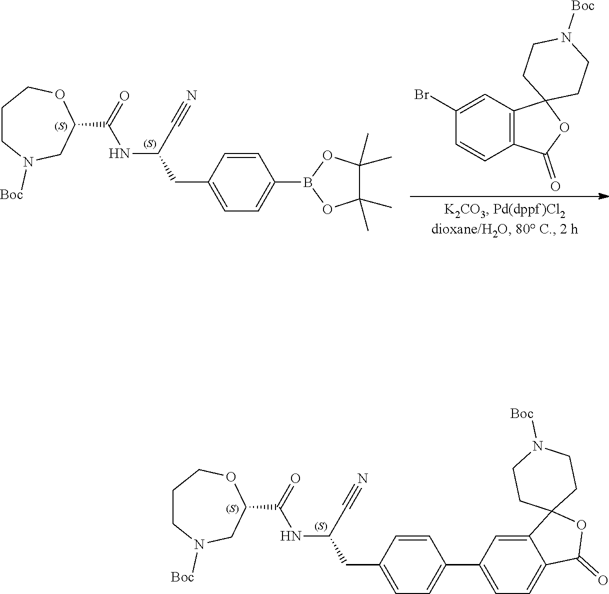

A solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (120 mg, 0.24 mmol, 1 equiv), tert-butyl 6-bromo-3-oxospiro[2-benzofuran-1,4′-piperidine]-1′-carboxylate (92 mg, 0.24 mmol, 1 equiv), K₂CO₃ (66 mg, 0.48 mmol, 2 equiv) and Pd(dppf)Cl₂CH₂Cl₂ (19 mg, 0.02 mmol, 0.1 equiv) in dioxane (5 mL), H₂O (0.5 mL) was stirred for 2 h at 80° C. under nitrogen atmosphere. The residue was purified by silica gel column chromatography, eluted with PE/THF (1:1) to afford tert-butyl 6-{4-[(2S)-2-{[(2S)-4-(tert-butoxycarbonyl)-1,4-oxazepan-2-yl]formamido}-2-cyanoethyl]phenyl}-3-oxospiro[2-benzofuran-1,4′-piperidine]-1′-carboxylate (110 mg, 67.84%) as a light yellow oil. LCMS (ES, m/z): [M+H]⁺: 675.

### Step 2. Synthesis of (2S,3aS,6aR)—N-[(1S)-1-cyano-2-{2-fluoro-4-[2-oxo-3-(2-propoxyethyl)-1,3-benzoxazol-5-yl]phenyl}ethyl]-hexahydro-1H-furo[3,4-b]pyrrole-2-carboxamide

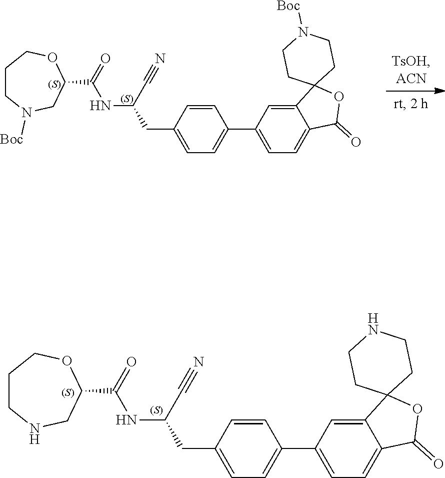

Into a 50 mL round-bottom flask were added tert-butyl 6-{4-[(2S)-2-{[(2S)-4-(tert-butoxycarbonyl)-1,4-oxazepan-2-yl]formamido}-2-cyanoethyl]phenyl}-3-oxospiro[2-benzofuran-1,4′-piperidine]-1′-carboxylate (110 mg, 0.16 mmol, 1 equiv), TsOH·H₂O (93 mg, 0.49 mmol, 3 equiv) and ACN (3 mL) at room temperature. The resulting mixture was stirred for additional 2 h at room temperature. The residue was purified by reversed-phase flash chromatography with the following conditions: column, C18 silica gel; mobile phase, MeCN in Water (0.1% NH₃·H₂O), 10% to 40% gradient in 10 min; detector, UV 254 nm. This resulted in Compound 24 (19.8 mg, 25.59%) as a white solid. LCMS (ES, m/z): [M+H]⁺: 475.5. ¹H NMR (400 MHz, DMSO-d₆) δ 8.63 (d, J=8.6 Hz, 1H), 7.98 (d, J=1.2 Hz, 1H), 7.87 (d, J=1.1 Hz, 2H), 7.81-7.75 (m, 2H), 7.45 (d, J=8.2 Hz, 2H), 5.06 (td, J=8.6, 7.1 Hz, 1H), 4.00 (dd, J=7.9, 3.7 Hz, 1H), 3.85 (ddd, J=12.5, 6.1, 4.4 Hz, 1H), 3.73 (ddd, J=12.1, 7.7, 4.0 Hz, 1H), 3.31-3.17 (m, 2H), 3.07-2.96 (m, 3H), 2.87 (t, J=12.1 Hz, 2H), 2.76 (dt, J=12.1, 5.5 Hz, 1H), 2.66-2.51 (m, 2H), 2.24-2.13 (m, 2H), 1.74-1.67 (m, 2H), 1.57 (d, J=13.5 Hz, 2H).

### Example 85: Synthesis of (2S)—N-[(1S)-1-cyano-2-(4-{3-oxospiro[2-benzofuran-1,3′-pyrrolidin]-6-yl}phenyl)ethyl]-1,4-oxazepane-2-carboxamide (Compound 25)

### Step 1. Synthesis of tert-butyl (2S)-2-{[(1S)-1-cyano-2-(4-{1-methyl-3′-oxospiro[azetidine-3,1′-[2]benzofuran]-6′-yl}phenyl)ethyl]carbamoyl}-1,4-oxazepane-4-carboxylate

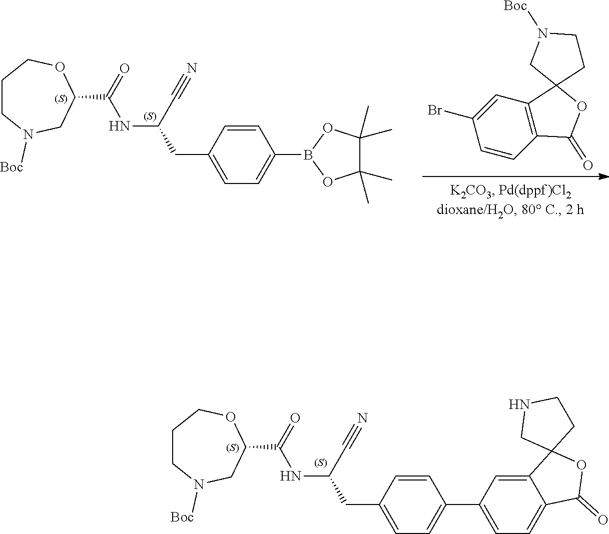

A solution of tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (150 mg, 0.30 mmol, 1.0 equiv), tert-butyl 6-bromo-3-oxospiro[2-benzofuran-1,3′-pyrrolidine]-1′-carboxylate (111 mg, 0.30 mmol, 1.0 equiv) and K₂CO₃ (83 mg, 0.60 mmol, 2.0 equiv), Pd(dppf)Cl₂ (22 mg, 0.03 mmol, 0.1 equiv) in 1,4-dioxane (5 mL), H₂O (0.5 mL) was stirred for 2 h at 80° C. under nitrogen atmosphere. The residue was purified by silica gel column chromatography, eluted with PE/THF (1:1) to afford tert-butyl (2S)-2-{[(1S)-1-cyano-2-(4-{1-methyl-3′-oxospiro[azetidine-3,1′-[2]benzofuran]-6′-yl}phenyl)ethyl]carbamoyl}-1,4-oxazepane-4-carboxylate (110 mg, 81.6%) as a yellow oil. LCMS (ES, m/z): [M+H]⁺: 561.

### Step 2. Synthesis of (2S)—N-[(1S)-1-cyano-2-(4-{3-oxospiro[2-benzofuran-1,3′-pyrrolidin]-6-yl}phenyl)ethyl]-1,4-oxazepane-2-carboxamide

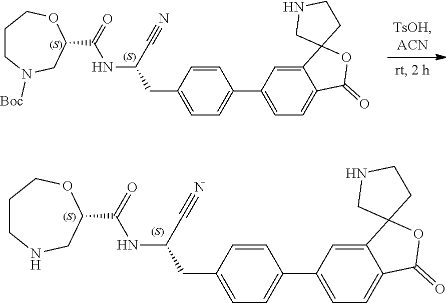

Into a 50 mL round-bottom flask were added tert-butyl (2S)-2-{[(1S)-1-cyano-2-(4-{3-oxospiro[2-benzofuran-1,3′-pyrrolidin]-6-yl}phenyl)ethyl]carbamoyl}-1,4-oxazepane-4-carboxylate (110 mg, 0.19 mmol, 1.0 equiv) and TsOH (101 mg, 0.58 mmol, 3.0 equiv) in ACN (3 mL) at room temperature. The resulting mixture was stirred for additional 3 h at room temperature. The residue was purified by reversed-phase flash chromatography with the following conditions: column, C18 silica gel; mobile phase, MeCN in Water (0.1% NH₃·H₂O), 10% to 80% gradient in 10 min; detector, UV 254 nm. This resulted in Compound 25 (15.0 mg, 16.60%) as a white solid. LCMS (ES, m/z): [M+H]⁺: 461.6. ¹H NMR (400 MHz, DMSO-d₆) δ 8.63 (d, J=8.5 Hz, 1H), 8.06 (s, 1H), 7.94-7.82 (m, 2H), 7.81-7.74 (m, 2H), 7.45 (d, J=8.1 Hz, 2H), 5.06 (q, J=8.3 Hz, 1H), 4.00 (dd, J=8.0, 3.7 Hz, 1H), 3.85 (ddd, J=12.4, 6.2, 4.7 Hz, 1H), 3.73 (ddd, J=12.0, 7.6, 4.1 Hz, 1H), 3.30-3.10 (m, 6H), 3.04 (dd, J=14.3, 3.8 Hz, 1H), 2.77 (dt, J=11.9, 5.3 Hz, 1H), 2.67-2.52 (m, 2H), 2.39 (dt, J=16.2, 8.6 Hz, 1H), 2.24-2.16 (m, 1H), 1.83-1.62 (m, 2H).

### Example 86: Synthesis of (2S)—N-[(1S)-1-cyano-2-(4-{1′-methyl-2,3-dihydrospiro[indene-1,4′-piperidin]-5-yl}phenyl)ethyl]-1,4-oxazepane-2-carboxamide (Compound 26)

### Step 1. Synthesis of tert-butyl 6-chlorospiro[indene-1,4′-piperidine]-1′-carboxylate and tert-butyl 6-chlorospiro[indene-1,4′-piperidine]-1′-carboxylate

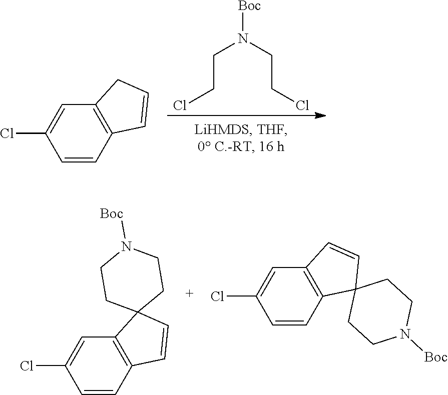

To a stirred solution of 5-chloro-3H-indene (5.0 g, 33.20 mmol, 1.0 equiv) in THF (50 mL) was added LiHMDS (1 M in TIF) (83.0 mL, 83.00 mmol, 2.5 equiv) dropwise at 0° C. under nitrogen atmosphere. The resulting mixture was stirred for 1 h at 0° C. under nitrogen atmosphere. To the above mixture was added tert-butyl N,N-bis(2-chloroethyl)carbamate (8.84 g, 36.52 mmol, 1.1 equiv) dropwise at 0° C. The resulting mixture was stirred for additional 15 h at room temperature. The reaction was quenched by the addition of sat. NH₄Cl (aq.) (20 mL) at 0° C., extracted with EtOAc (3×50 mL). The combined organic layer was washed with brine (50 mL), dried over anhydrous Na₂SO₄. After filtration, the filtrate was concentrated under reduced pressure. The residue was purified by silica gel column chromatography, eluted with PE/EA (3:1) to afford the mixture of tert-butyl 6-chlorospiro[indene-1,4′-piperidine]-1′-carboxylate and tert-butyl 6-chlorospiro[indene-1,4′-piperidine]-1′-carboxylate (2.2 g, 41.4%) as a colorless oil.

### Step 2. Synthesis of tert-butyl 5-chloro-2,3-dihydrospiro[indene-1,4′-piperidine]-1′-carboxylate and tert-butyl 6-chloro-2,3-dihydrospiro[indene-1,4′-piperidine]-1′-carboxylate

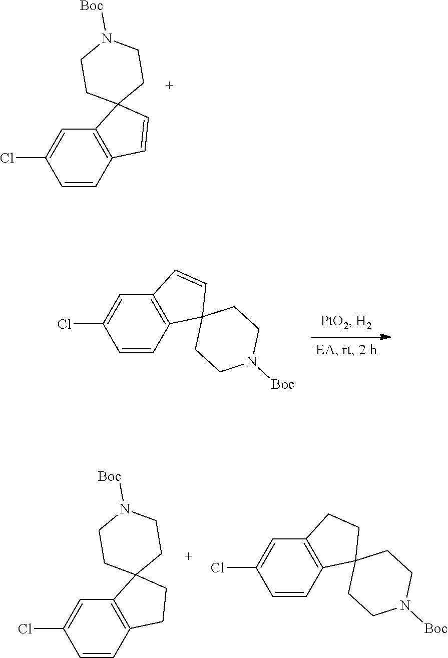 [SMILES: CC(C)(C)OC(=O)N1CCC2(C=Cc3ccc(Cl)cc32)CC1 ; CC(C)(C)OC(=O)N1CCC2(C=Cc3cc(Cl)ccc32)CC1 ; CC(C)(C)OC(=O)N1CCC2(CCc3ccc(Cl)cc32)CC1 ; CC(C)(C)OC(=O)N1CCC2(CCc3cc(Cl)ccc32)CC1]

To a stirred mixture of tert-butyl 5-chlorospiro[indene-1,4′-piperidine]-1′-carboxylate and tert-butyl 6-chlorospiro[indene-1,4′-piperidine]-1′-carboxylate (4.4 g, 13.76 mmol, 1.0 equiv) in EA (60 mL) was added PtO₂ (0.5 g). The resulting mixture was stirred for 2 h at room temperature under hydrogen atmosphere (3 atm). The resulting mixture was filtered, the filter cake was washed with EA (50 mL). The filtrate was concentrated under reduced pressure. The mixture of tert-butyl 5-chloro-2,3-dihydrospiro[indene-1,4′-piperidine]-1′-carboxylate and tert-butyl 6-chloro-2,3-dihydrospiro[indene-1,4′-piperidine]-1′-carboxylate (4.2 g, 95%) was used in the next step directly without further purification.

### Step 3. Synthesis of 5-chloro-2,3-dihydrospiro[indene-1,4′-piperidine] and 6-chloro-2,3-dihydrospiro[indene-1,4′-piperidine]trifluoroacetaldehyde

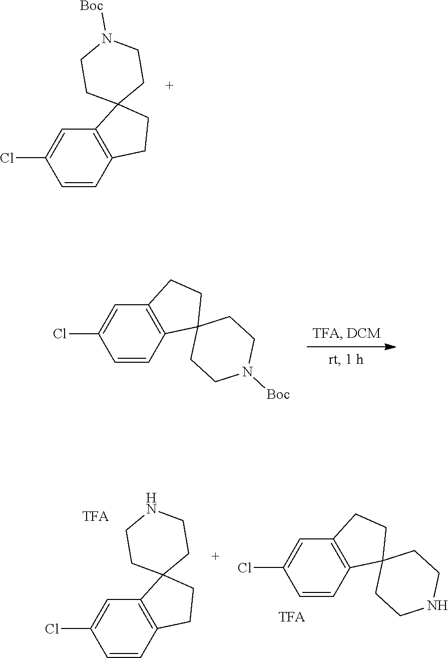 [SMILES: CC(C)(C)OC(=O)N1CCC2(CCc3ccc(Cl)cc32)CC1 ; CC(C)(C)OC(=O)N1CCC2(CCc3cc(Cl)ccc32)CC1 ; Clc1ccc2c(c1)C1(CCNCC1)CC2 ; Clc1ccc2c(c1)CCC21CCNCC1]

To a stirred mixture of tert-butyl 5-chloro-2,3-dihydrospiro[indene-1,4′-piperidine]-1′-carboxylate and tert-butyl 6-chloro-2,3-dihydrospiro[indene-1,4′-piperidine]-1′-carboxylate (0.70 g, 2.18 mmol, 1.0 equiv) in DCM (15 mL) was added TFA (3 mL). The resulting mixture was stirred for 1 h at room temperature. The resulting mixture was concentrated under reduced pressure. The mixture of 5-chloro-2,3-dihydrospiro[indene-1,4′-piperidine] and 6-chloro-2,3-dihydrospiro[indene-1,4′-piperidine]trifluoroacetaldehyde (1.0 g crude product) was used in the next step directly without further purification. LCMS (ES) [M-TFA+H]⁺ m/z: 222.

### Step 4. Synthesis of 5-chloro-1′-methyl-2,3-dihydrospiro[indene-1,4′-piperidine] and 6-chloro-1′-methyl-2,3-dihydrospiro[indene-1,4′-piperidine]

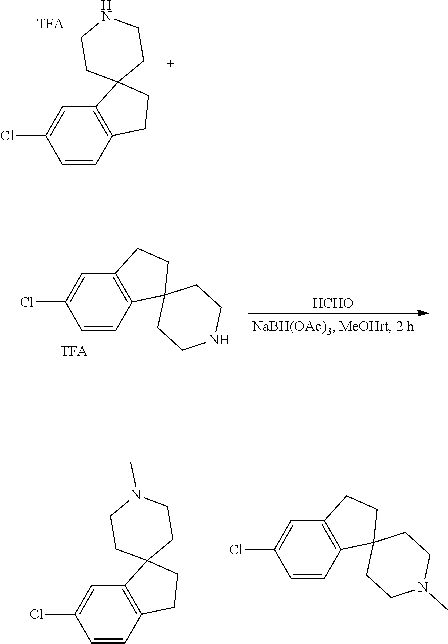 [SMILES: Clc1ccc2c(c1)C1(CCNCC1)CC2 ; Clc1ccc2c(c1)CCC21CCNCC1 ; "HCHO" ; CN1CCC2(CCc3ccc(Cl)cc32)CC1 ; CN1CCC2(CCc3cc(Cl)ccc32)CC1]

To a stirred mixture of 5-chloro-2,3-dihydrospiro[indene-1,4′-piperidine] and 6-chloro-2,3-dihydrospiro[indene-1,4′-piperidine](1.0 g, 4.55 mmol, 1.0 equiv) in MeOH (10 mL) was added HCHO in H₂O (4.52 g, 23.10 mmol, 10 equiv, 30%). The resulting mixture was stirred for 2 h at room temperature. To the above mixture was added NaBH(OAc)₃ (2.86 g, 13.52 mmol, 3.0 equiv). The resulting mixture was stirred for additional 12 h at room temperature. The mixture was basified to pH 8 with saturated NaHCO₃ (aq.), extracted with CH₂Cl₂ (3×30 mL). The combined organic layer was washed with brine (50 mL), dried over anhydrous Na₂SO₄. After filtration, the filtrate was concentrated under reduced pressure. The residue was purified by silica gel column chromatography, eluted with CH₂Cl₂/MeOH (18:1) to afford 5-chloro-1′-methyl-2,3-dihydrospiro[indene-1,4′-piperidine](320 mg, 30%) as a white solid and 6-chloro-1′-methyl-2,3-dihydrospiro[indene-1,4′-piperidine](350 mg, 33%) as a white solid. LCMS (ES) [M+H]⁺ m/z: 236.

### Step 5. Synthesis of tert-butyl (2S)-2-{[(1S)-1-cyano-2-(4-{1′-methyl-2,3-dihydrospiro[indene-1,4′-piperidin]-5-yl}phenyl)ethyl]carbamoyl}-1,4-oxazepane-4-carboxylate

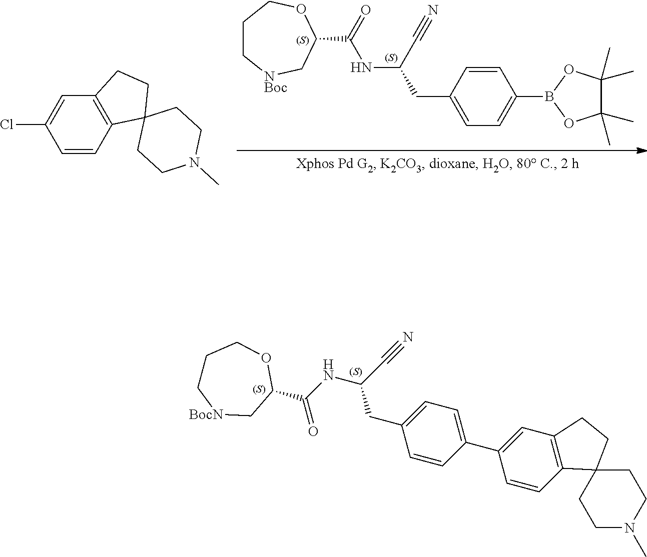

To a stirred solution of 5-chloro-1′-methyl-2,3-dihydrospiro[indene-1,4′-piperidine](80 mg, 0.34 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (186 mg, 0.37 mmol, 1.1 equiv) in dioxane (1 mL) and H₂O (0.1 mL) were added K₂CO₃ (93 mg, 0.68 mmol, 2.0 equiv) and 2nd Generation XPhos Pd (26 mg, 0.03 mmol, 0.1 equiv). The resulting mixture was stirred for 2 h at 80° C. under nitrogen atmosphere. The resulting mixture was concentrated under reduced pressure. The residue was purified by silica gel column chromatography, eluted with PE/THF (4:1) to afford tert-butyl (2S)-2-{[(1S)-1-cyano-2-(4-{1′-methyl-2,3-dihydrospiro[indene-1,4′-piperidin]-5-yl}phenyl)ethyl]carbamoyl}-1,4-oxazepane-4-carboxylate (100 mg, 51.5%) as a white solid. LCMS (ES) [M+H]⁺ m/z: 573.

### Step 6. Synthesis of (2S)—N-[(1S)-1-cyano-2-(4-{1′-methyl-2,3-dihydrospiro[indene-1,4′-piperidin]-5-yl}phenyl)ethyl]-1,4-oxazepane-2-carboxamide

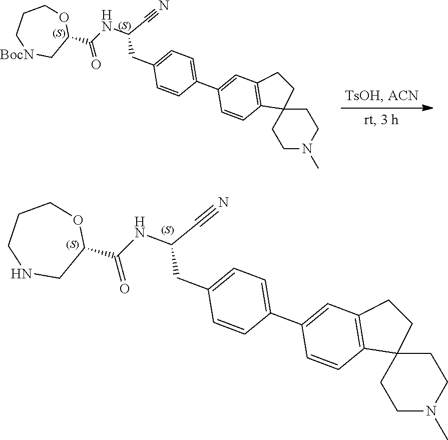

To a stirred solution of tert-butyl (2S)-2-{[(1S)-1-cyano-2-(4-{1′-methyl-2,3-dihydrospiro[indene-1,4′-piperidin]-5-yl}phenyl)ethyl]carbamoyl}-1,4-oxazepane-4-carboxylate (90 mg, 0.16 mmol, 1.0 equiv) in ACN (1 mL) was added TsOH (81 mg, 0.47 mmol, 3.0 equiv). The resulting mixture was stirred for 2 h at room temperature. The residue was purified by reversed-phase flash chromatography with the following conditions: column, C¹⁸ silica gel; mobile phase, MeCN in Water (0.1% NH₃·H₂O), 5% to 60% gradient in 15 min; detector, UV 254 nm. This resulted in Compound 26 (20 mg, 27%) as a white solid. LCMS (ES) [M+H]⁺ m/z: 473.2. ¹H NMR (300 MHz, DMSO-d₆) δ 8.62 (d, J=8.6 Hz, 1H), 7.56 (d, J=8.2 Hz, 2H), 7.48-7.39 (m, 2H), 7.35 (d, J=8.2 Hz, 2H), 7.25 (d, J=7.8 Hz, 1H), 5.09-4.93 (m, 1H), 3.99 (dd, J=7.9, 3.7 Hz, 1H), 3.94-3.78 (m, 1H), 3.79-3.65 (m, 1H), 3.28-3.10 (m, 2H), 3.02 (dd, J=14.3, 3.7 Hz, 1H), 2.90 (t, J=7.3 Hz, 2H), 2.83-2.67 (m, 3H), 2.67-2.50 (m, 2H), 2.22 (s, 3H), 2.15-2.00 (m, 2H), 1.98 (t, J=7.3 Hz, 2H), 1.93-1.76 (m, 2H), 1.78-1.66 (m, 2H), 1.45 (d, J=12.5 Hz, 2H).

### Example 87: Synthesis of (2S)—N-[(1S)-1-cyano-2-{4-[1-(oxetan-3-yl)-3′-oxospiro[azetidine-3,1′-[2]benzofuran]-6′-yl]phenyl}ethyl]-1,4-oxazepane-2-carboxamide (Compound 27)

### Step 1. Synthesis of 6′-bromo-1-(oxetan-3-yl)spiro[azetidine-3,1′-[2]benzofuran]-3′-one

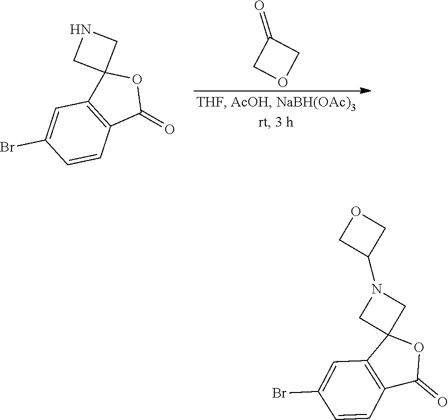 [SMILES: O=C1OC2(CNC2)c2cc(Br)ccc21 ; O=C1COC1 ; O=C1OC2(CN(C3COC3)C2)c2cc(Br)ccc21]

To a stirred solution of 6′-bromospiro[azetidine-3,1′-[2]benzofuran]-3′-one (500 mg, 1.968 mmol, 1.0 equiv) and 3-oxetanone (567 mg, 7.872 mmol, 4.0 equiv) in THF (6 mL) was added AcOH (11 mg, 0.197 mmol, 0.1 equiv). The resulting mixture was stirred for 1 h at room temperature. To the above mixture was added NaBH(OAc)₃ (1251 mg, 5.904 mmol, 3.0 equiv) in portions. The resulting mixture was stirred for additional 2 h at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/THF (3:1) to afford 6′-bromo-1-(oxetan-3-yl)spiro[azetidine-3,1′-[2]benzofuran]-3′-one (300 mg, 49.15%) as a white solid. LCMS (ES) [M+H]⁺ m/z: 310.

### Step 2. Synthesis of tert-butyl (2S)-2-{[(1S)-1-cyano-2-{4-[1-(oxetan-3-yl)-3′-oxospiro[azetidine-3,1′-[2]benzofuran]-6′-yl]phenyl}ethyl]carbamoyl}-1,4-oxazepane-4-carboxylate

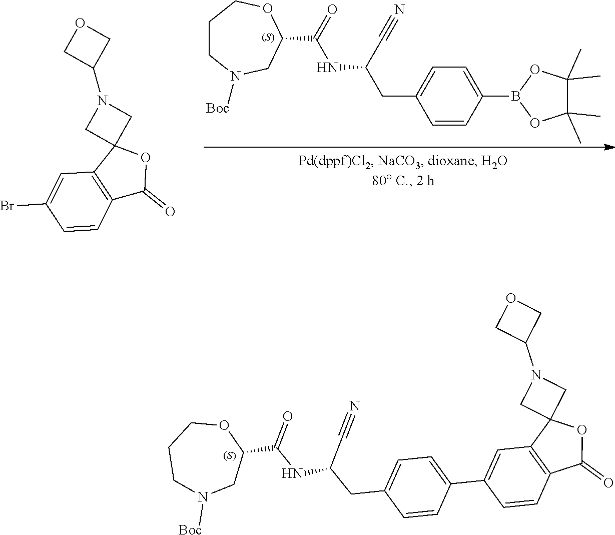

To a stirred solution of 6′-bromo-1-(oxetan-3-yl)spiro[azetidine-3,1′-[2]benzofuran]-3′-one (70 mg, 0.226 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (112 mg, 0.226 mmol, 1.0 equiv) in dioxane (2.0 mL) and H₂O (0.2 mL) were added Na₂CO₃ (47 mg, 0.452 mmol, 2.0 equiv) and Pd(dppf)Cl₂ (16 mg, 0.023 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (2:1) to afford tert-butyl (2S)-2-{[(1S)-1-cyano-2-{4-[1-(oxetan-3-yl)-3′-oxospiro[azetidine-3,1′-[2]benzofuran]-6′-yl]phenyl}ethyl]carbamoyl}-1,4-oxazepane-4-carboxylate (120 mg, 88.22%) as a light yellow solid. LCMS (ES) [M+H]⁺ m/z: 603.

### Step 3. Synthesis of (2S)—N-[(1S)-1-cyano-2-{4-[1-(oxetan-3-yl)-3′-oxospiro[azetidine-3,1′-[2]benzofuran]-6′-yl]phenyl}ethyl]-1,4-oxazepane-2-carboxamide

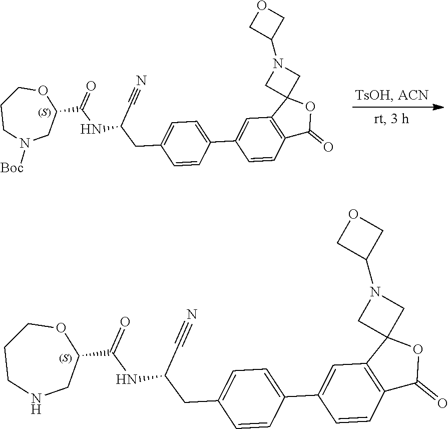

To a stirred solution of tert-butyl (2S)-2-{[(1S)-1-cyano-2-{4-[1-(oxetan-3-yl)-3′-oxospiro[azetidine-3,1′-[2]benzofuran]-6′-yl]phenyl}ethyl]carbamoyl}-1,4-oxazepane-4-carboxylate (120 mg, 0.199 mmol, 1.0 equiv) in ACN (2.0 mL) was added TsOH (102 mg, 0.597 mmol, 3.0 equiv). The resulting mixture was stirred for 3 h at room temperature. The mixture was purified by reversed-phase flash chromatography with the following conditions: column, C18 silica gel; mobile phase, MeCN in Water (0.1% NH₃·H₂O), 10% to 50% gradient in 10 min; detector, UV 254 nm. This resulted in Compound 27 (30 mg, 29.98%) as a white solid. LCMS (ES) [M+H]⁺ m/z: 503.2. ¹H NMR (400 MHz, DMSO-d₆) δ 8.65 (d, J=8.6 Hz, 1H), 8.17 (t, J=1.1 Hz, 1H), 7.95-7.85 (m, 2H), 7.84-7.76 (m, 2H), 7.47 (d, J=8.2 Hz, 2H), 5.12-5.01 (m, 1H), 4.64 (t, J=6.7 Hz, 2H), 4.49 (ddd, J=6.5, 5.1, 1.2 Hz, 2H), 4.04-3.93 (m, 2H), 3.90-3.67 (m, 6H), 3.32-3.17 (m, 2H), 3.04 (dd, J=14.2, 3.7 Hz, 1H), 2.83-2.73 (m, 1H), 2.67-2.57 (m, 1H), 2.57-2.52 (m, 1H), 1.83-1.67 (m, 2H).

### Example 88: Synthesis of (2S)—N-[(1S)-1-cyano-2-{4-[1-methyl-1′-(oxetan-3-yl)-2-oxospiro[indole-3,3′-piperidin]-5-yl]phenyl}ethyl]-1,4-oxazepane-2-carboxamide (Compound 28)

### Step 1. Synthesis of 5-bromo-1-methyl-1′-(oxetan-3-yl)spiro[indole-3,3′-piperidin]-2-one

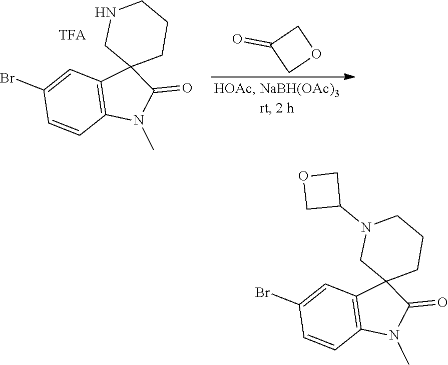 [SMILES: CN1C(=O)C2(CCCNC2)c2cc(Br)ccc21 ; O=C1COC1 ; CN1C(=O)C2(CCCN(C3COC3)C2)c2cc(Br)ccc21]

To a stirred solution of 5-bromo-1-methylspiro[indole-3,3′-piperidin]-2-one trifluoroacetic acid salt (200 mg, 0.49 mmol, 1.0 equiv) and 3-oxetanone (105 mg, 1.47 mmol, 3.0 equiv) in THF (4 mL) were added HOAc (2 mg, 0.05 mmol, 0.1 equiv). The resulting mixture was stirred for 1 h at room temperature. To the above mixture was added NaBH(OAc)₃ (310 mg, 1.47 mmol, 3.0 equiv) in portions at room temperature. The resulting mixture was stirred for additional 1 h at room temperature. The mixture was basified to pH 8 with saturated NaHCO₃ (aq.), extracted with CH₂Cl₂ (3×10 mL). The combined organic layer was washed with brine (10 mL), dried over anhydrous Na₂SO₄. After filtration, the filtrate was concentrated under reduced pressure. This resulted in 5-bromo-1-methyl-1′-(oxetan-3-yl)spiro[indole-3,3′-piperidin]-2-one (150 mg, 87.3%) as white solid. LCMS (ES) [M+1]⁺ m/z: 351.

### Step 2. Synthesis of tert-butyl (2S)-2-{[(1S)-1-cyano-2-{4-[1-methyl-1′-(oxetan-3-yl)-2-oxospiro[indole-3,3′-piperidin]-5-yl]phenyl}ethyl]carbamoyl}-1,4-oxazepane-4-carboxylate

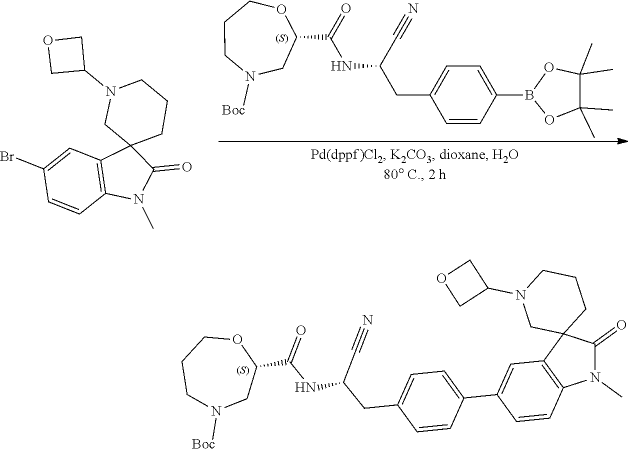

To a stirred solution of 5-bromo-1-methyl-1′-(oxetan-3-yl)spiro[indole-3,3′-piperidin]-2-one (118 mg, 0.34 mmol, 1.3 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (130 mg, 0.26 mmol, 1.0 equiv) in dioxane (3 mL) and H₂O (0.3 mL) were added Na₂CO₃ (55 mg, 0.52 mmol, 2.0 equiv) and Pd(dppf)Cl₂ (19 mg, 0.03 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (1:1) to afford tert-butyl (2S)-2-{[(1S)-1-cyano-2-{4-[1-methyl-1′-(oxetan-3-yl)-2-oxospiro[indole-3,3′-piperidin]-5-yl]phenyl}ethyl]carbamoyl}-1,4-oxazepane-4-carboxylate (100 mg, 59.6%) as white foam solid. LCMS (ES) [M+1]⁺ m/z: 644.

### Step 3. Synthesis of (2S)—N-[(1S)-1-cyano-2-{4-[1-methyl-1′-(oxetan-3-yl)-2-oxospiro[indole-3,3′-piperidin]-5-yl]phenyl}ethyl]-1,4-oxazepane-2-carboxamide

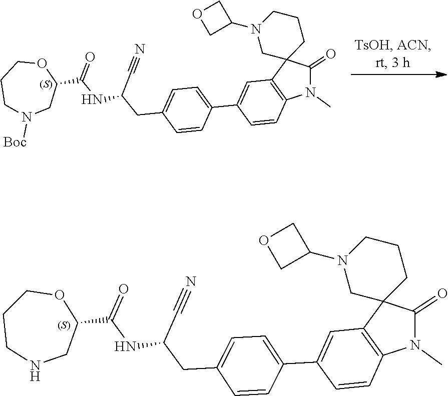

To a stirred solution of tert-butyl (2S)-2-{[(1S)-1-cyano-2-{4-[1-methyl-1′-(oxetan-3-yl)-2-oxospiro[indole-3,3′-piperidin]-5-yl]phenyl}ethyl]carbamoyl}-1,4-oxazepane-4-carboxylate (100 mg, 0.16 mmol, 1.0 equiv) in ACN (2 mL) was added TsOH (80 mg, 0.46 mmol, 3.0 equiv). The resulting mixture was stirred for 3 h at room temperature. The reaction solution was purified by Prep-HPLC with the following conditions: Column, XBridge Prep C18 OBD Column, 19*150 mm, 5 um; mobile phase, Water (0.1% NH₃H₂O) and ACN (10% Phase B up to 80% in 20 min, Detector, UV 254 nm. This resulted in Compound 28 (30 mg, 35.5%) as white solid. LCMS (ES) [M+1]⁺ m/z: 544.6. ¹H NMR (300 MHz, DMSO-d₆) δ 8.62 (d, J=8.5 Hz, 1H), 8.11 (dd, J=3.4, 1.9 Hz, 1H), 7.62-7.55 (m, 3H), 7.39 (d, J=8.1 Hz, 2H), 7.12 (d, J=8.2 Hz, 1H), 5.10-4.96 (m, 1H), 4.60-4.45 (m, 2H), 4.41 (t, J=6.3 Hz, 1H), 4.14 (t, J=6.1 Hz, 1H), 4.02-3.97 (m, 1H), 3.91-3.79 (m, 1H), 3.76-3.70 (m, 1H), 3.45 (t, J=6.3 Hz, 1H), 3.25-3.13 (m, 5H), 3.03 (dd, J=14.3, 3.8 Hz, 1H), 2.93-2.84 (m, 1H), 2.76-2.70 (m, 1H), 2.67-2.56 (m, 2H), 2.41 (d, J=10.8 Hz, 1H), 2.22 (d, J=10.9 Hz, 1H), 2.08-1.96 (m, 2H), 1.83-1.68 (m, 4H), 1.50 (d, J=12.2 Hz, 1H).

### Example 89: Synthesis of (2S)—N-[(1S)-1-cyano-2-{4-[1-methyl-1′-(oxetan-3-yl)-2-oxospiro[indole-3,4′-piperidin]-5-yl]phenyl}ethyl]-1,4-oxazepane-2-carboxamide (Compound 29)

### Step 1. Synthesis of 5-bromo-1-methyl-1′-(oxetan-3-yl)spiro[indole-3,4′-piperidin]-2-one

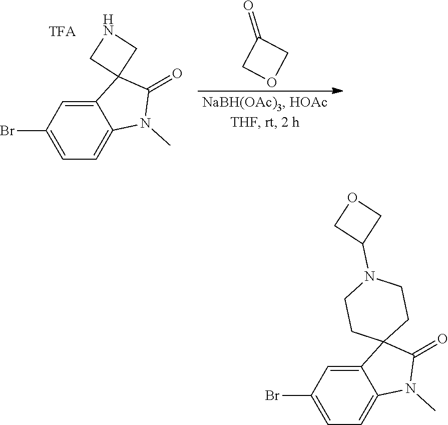 [SMILES: CN1C(=O)C2(CNC2)c2cc(Br)ccc21 ; O=C1COC1 ; CN1C(=O)C2(CCN(C3COC3)CC2)c2cc(Br)ccc21]

To a stirred solution of 5-bromo-1-methylspiro[indole-3,4′-piperidin]-2-one; trifluoroacetic acid (400 mg, 0.98 mmol, 1.0 equiv) and 3-oxetanone (211 mg, 2.94 mmol, 3.0 equiv) in THF (6 mL) were added HOAc (5 mg, 0.10 mmol, 0.1 equiv). The resulting mixture was stirred for 1 h at room temperature. To the above mixture was added NaBH(OAc)₃ (621 mg, 2.94 mmol, 3.0 equiv) in portions at room temperature. The resulting mixture was stirred for additional 1 h at room temperature. The mixture was basified to pH 9 with saturated NaHCO₃ (aq.). The resulting mixture was extracted with CH₂Cl₂ (3×20 mL). The combined organic layer was washed with brine (20 mL), dried over anhydrous Na₂SO₄. After filtration, the filtrate was concentrated under reduced pressure. This resulted in 5-bromo-1-methyl-1′-(oxetan-3-yl)spiro[indole-3,4′-piperidin]-2-one (300 mg, 87.3%) as light yellow solid. LCMS (ES) [M+1]⁺ m/z: 351.

### Step 2. Synthesis of tert-butyl (2S)-2-{[(1S)-1-cyano-2-{4-[1-methyl-1′-(oxetan-3-yl)-2-oxospiro[indole-3,4′-piperidin]-5-yl]phenyl}ethyl]carbamoyl}-1,4-oxazepane-4-carboxylate

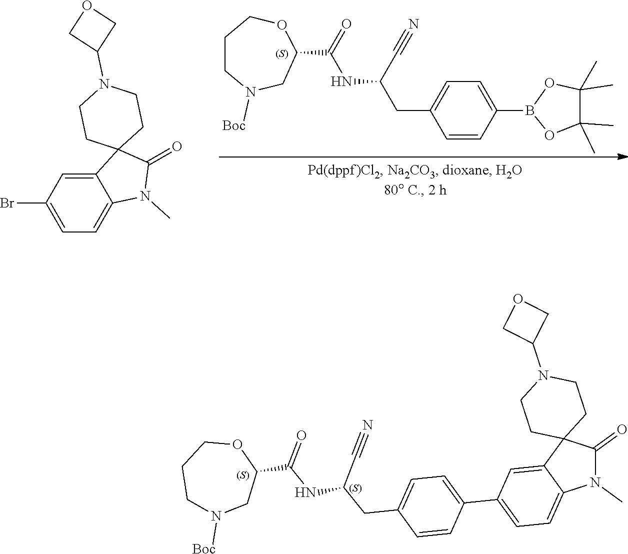

To a stirred solution of 5-bromo-1-methyl-1′-(oxetan-3-yl)spiro[indole-3,4′-piperidin]-2-one (84 mg, 0.24 mmol, 1.2 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (100 mg, 0.20 mmol, 1.0 equiv) in dioxane (2 mL) and H₂O (0.2 mL) were added Pd(dppf)Cl₂ (14 mg, 0.02 mmol, 0.1 equiv) and Na₂CO₃ (42 mg, 0.40 mmol, 2.0 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (1:1) to afford tert-butyl (2S)-2-{[(1S)-1-cyano-2-{4-[1-methyl-1′-(oxetan-3-yl)-2-oxospiro[indole-3,4′-piperidin]-5-yl]phenyl}ethyl]carbamoyl}-1,4-oxazepane-4-carboxylate (100 mg, 77.5%) as light yellow solid. LCMS (ES) [M+1]⁺ m/z: 644.

### Step 3. Synthesis of (2S)—N-[(1S)-1-cyano-2-{4-[1-methyl-1′-(oxetan-3-yl)-2-oxospiro[indole-3,4′-piperidin]-5-yl]phenyl}ethyl]-1,4-oxazepane-2-carboxamide

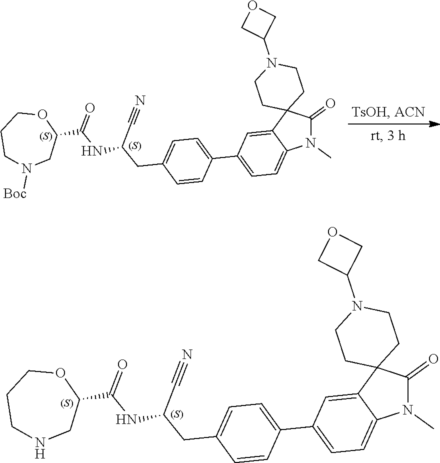

To a stirred solution of tert-butyl (2S)-2-{[(1S)-1-cyano-2-{4-[1-methyl-1′-(oxetan-3-yl)-2-oxospiro[indole-3,4′-piperidin]-5-yl]phenyl}ethyl]carbamoyl}-1,4-oxazepane-4-carboxylate (100 mg, 0.16 mmol, 1.0 equiv) in ACN (2 mL) was added TsOH (80 mg, 0.46 mmol, 3.0 equiv). The resulting mixture was stirred for 3 h at room temperature. The reaction solution was purified by Prep-HPLC with the following conditions: Column, XBridge Prep C18 OBD Column, 19*150 mm, 5 um; mobile phase, Water (0.1% NH₃H₂O) and ACN (10% Phase B up to 80% in 20 min); Detector, UV 254 nm. This resulted in Compound 29 (30 mg, 35.5%) as white solid. LCMS (ES) [M+1]⁺ m/z: 544.3. ¹H NMR (300 MHz, DMSO-d₆) δ 8.62 (d, J=8.6 Hz, 1H), 7.74 (d, J=1.8 Hz, 1H), 7.67-7.54 (m, 3H), 7.36 (d, J=8.2 Hz, 2H), 7.09 (d, J=8.2 Hz, 1H), 5.07-4.98 (m, 1H), 4.59 (t, J=6.4 Hz, 2H), 4.48 (t, J=6.1 Hz, 2H), 4.00 (dd, J=7.8, 3.7 Hz, 1H), 3.89-3.81 (m, 1H), 3.77-3.68 (m, 1H), 3.64-3.55 (m, 1H), 3.20 (dd, J=7.9, 3.5 Hz, 2H), 3.15 (s, 3H), 3.04 (dd, J=14.3, 3.7 Hz, 1H), 2.83-2.52 (m, 7H), 1.98-1.61 (m, 6H).

### Example 90: Synthesis of (2S)—N-[(1S)-1-cyano-2-(4-{3-oxo-2H-spiro[indene-1,4′-piperidin]-6-yl}phenyl)ethyl]-1,4-oxazepane-2-carboxamide (Compound 30)

### Step 1. Synthesis of tert-butyl 6-{4-[(2S)-2-{[(2S)-4-(tert-butoxycarbonyl)-1,4-oxazepan-2-yl]formamido}-2-cyanoethyl]phenyl}-3-oxo-2H-spiro[indene-1,4′-piperidine]-1′-carboxylate

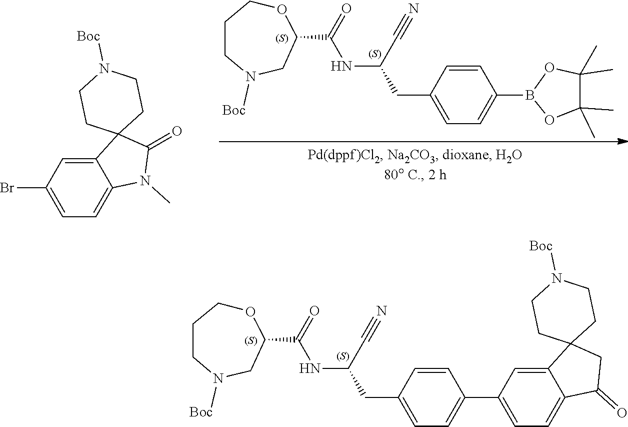

To a stirred solution of tert-butyl 6-bromo-3-oxo-2H-spiro[indene-1,4′-piperidine]-1′-carboxylate (150 mg, 0.394 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (196 mg, 0.394 mmol, 1.0 equiv) in dioxane (3 mL) and H₂O (0.3 mL) were added Pd(dppf)Cl₂ (28 mg, 0.039 mmol, 0.1 equiv) and Na₂CO₃ (83 mg, 0.788 mmol, 2.0 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (3:1) to afford tert-butyl 6-{4-[(2S)-2-{[(2S)-4-(tert-butoxycarbonyl)-1,4-oxazepan-2-yl]formamido}-2-cyanoethyl]phenyl}-3-oxo-2H-spiro[indene-1,4′-piperidine]-1′-carboxylate (200 mg, 75.3%) as a light yellow solid. LCMS (ES) [M+1]⁺ m/z: 673.

### Step 2. Synthesis of (2S)—N-[(1S)-1-cyano-2-(4-{3-oxo-2H-spiro[indene-1,4′-piperidin]-6-yl}phenyl)ethyl]-1,4-oxazepane-2-carboxamide

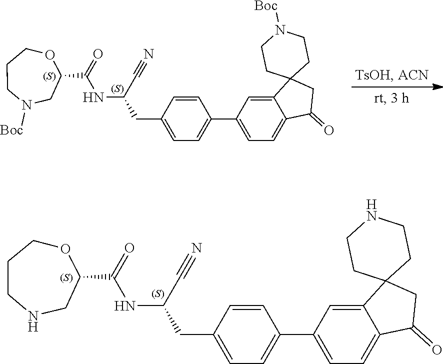

To a stirred solution of tert-butyl 6-{4-[(2S)-2-{[(2S)-4-(tert-butoxycarbonyl)-1,4-oxazepan-2-yl]formamido}-2-cyanoethyl]phenyl}-3-oxo-2H-spiro[indene-1,4′-piperidine]-1′-carboxylate (200 mg, 0.297 mmol, 1.0 equiv) in ACN (3 mL) was added TsOH (204 mg, 1.188 mmol, 4.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound 30 (40 mg, 28.4%) as a white solid. LCMS (ES) [M+1]⁺ m/z: 473.2. ¹H NMR (300 MHz, DMSO-d6) δ 8.63 (d, J=8.6 Hz, 1H), 7.91 (s, 1H), 7.80-7.69 (m, 3H), 7.66 (d, J=8.0 Hz, 1H), 7.43 (d, J=8.2 Hz, 2H), 5.13-4.99 (m, 1H), 4.00 (dd, J=7.9, 3.7 Hz, 1H), 3.91-3.78 (m, 1H), 3.72 (ddd, J=12.1, 7.4, 4.2 Hz, 1H), 3.28-3.18 (m, 2H), 3.09-2.91 (m, 3H), 2.83-2.51 (m, 7H), 2.11-1.93 (m, 2H), 1.79-1.64 (m, 2H), 1.40 (d, J=12.5 Hz, 2H).

### Example 91: Synthesis by (2S)—N-[(1S)-1-cyano-2-[4′-methoxy-3′-(1,2,4-triazol-4-yl)-[1,1′-biphenyl]-4-yl]ethyl]-1,4-oxazepane-2-carboxamide (Compound 31)

### Step 1. Synthesis of 4-(5-bromo-2-methoxyphenyl)-1,2,4-triazole

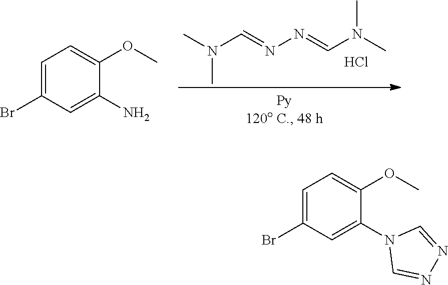

Into a 100 mL round-bottom flask were added 5-bromo-2-methoxyaniline (2 g, 9.90 mmol, 1.0 equiv) and (E)-N′-[(E)-N′—[(N, N-dimethylamino)methylidene]amino]-N,N-dimethylmethanimidamide (2.11 g, 14.85 mmol, 1.5 equiv) at room temperature. The resulting mixture was stirred for 48 h at 120° C. The mixture was allowed to cool down to room temperature. The resulting mixture was concentrated under reduced pressure. The residue was purified by silica gel column chromatography, eluted with PE/EA (1:1) to afford 4-(5-bromo-2-methoxyphenyl)-1,2,4-triazole (480 mg, 19.08%) as a white solid. LCMS (ES, m/z): [M+H]⁺: 255. ¹H NMR (300 MHz, DMSO-d6) δ 8.83 (s, 2H), 7.80 (d, J=2.4 Hz, 1H), 7.66 (dd, J=8.9, 2.5 Hz, 1H), 7.27 (d, J=8.9 Hz, 1H), 3.85 (s, 3H).

### Step 2. Synthesis of tert-butyl (2S)-2-{[(1S)-1-cyano-2-[4′-methoxy-3′-(1,2,4-triazol-4-yl)-[1,1′-biphenyl]-4-yl]ethyl]carbamoyl}-1,4-oxazepane-4-carboxylate

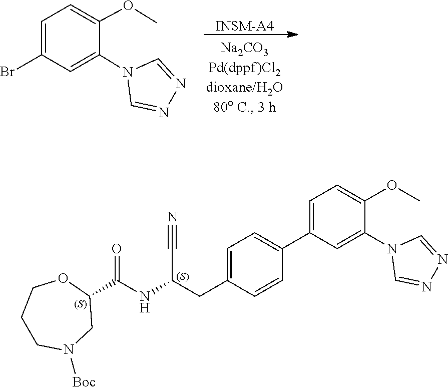

To a stirred mixture of 4-(5-bromo-2-methoxyphenyl)-1,2,4-triazole (60 mg, 0.24 mmol, 1.0 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (153.31 mg, 0.31 mmol, 1.3 equiv) in dioxane (3 mL) and H₂O (0.3 mL) were added Na₂CO₃ (50.06 mg, 0.47 mmol, 2.0 equiv) and Pd(dppf)Cl₂ (17.28 mg, 0.02 mmol, 0.1 equiv) in portion at room temperature under nitrogen atmosphere. The resulting mixture was stirred for additional 3 h at 80° C. The resulting mixture was filtered, the filter cake was washed with EtOAc (3×10 mL). The filtrate was concentrated under reduced pressure. The residue was purified by silica gel column chromatography, eluted with PE/THF (1:3) to afford tert-butyl (2S)-2-{[(1S)-1-cyano-2-[4′-methoxy-3′-(1,2,4-triazol-4-yl)-[1,1′-biphenyl]-4-yl]ethyl]carbamoyl}-1,4-oxazepane-4-carboxylate (100 mg, 77.47%) as a white semi-solid. LCMS (ES, m/z): [M+H]⁺: 547.

### Step 3. Synthesis of (2S)—N-[(1S)-1-cyano-2-[4′-methoxy-3′-(1,2,4-triazol-4-yl)-[1,1′-biphenyl]-4-yl]ethyl]-1,4-oxazepane-2-carboxamide

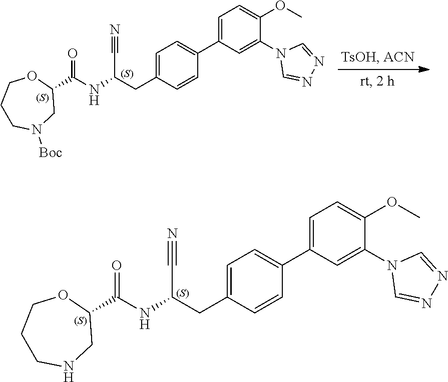

To a stirred solution of tert-butyl (2S)-2-{[(1S)-1-cyano-2-[4′-methoxy-3′-(1,2,4-triazol-4-yl)-[1,1′-biphenyl]-4-yl]ethyl]carbamoyl}-1,4-oxazepane-4-carboxylate (95 mg, 0.17 mmol, 1.0 equiv) in ACN (3 mL) were added TsOH·H₂O (99.17 mg, 0.52 mmol, 3.0 equiv) in portions at room temperature. The resulting mixture was stirred for additional 2 h at room temperature. The crude product was purified by Prep-HPLC with the following conditions (column, C18 silica gel; mobile phase, MeCN in Water (0.1% NH₃·H₂O), 20% to 40% gradient in 10 min; detector, UV 254 nm.) to afford Compound 31 (25 mg, 32.22%) as a white solid. LCMS (ES, m/z): [M+H]⁺: 447.3. ¹H NMR (300 MHz, DMSO-d₆) δ 8.87 (s, 2H), 8.59 (d, J=8.6 Hz, 1H), 7.81-7.73 (m, 2H), 7.68 (d, J=7.9 Hz, 2H), 7.36 (dd, J=8.6, 2.4 Hz, 3H), 5.00 (q, J=8.1 Hz, 1H), 3.97 (dd, J=7.9, 3.6 Hz, 1H), 3.87 (s, 3H), 3.81 (t, J=5.4 Hz, 1H), 3.76-3.66 (m, 1H), 3.22-3.12 (m, 2H), 3.01 (dd, J=14.2, 3.7 Hz, 1H), 2.78-2.69 (m, 1H), 2.64-2.52 (m, 2H), 1.70 (d, J=5.7 Hz, 2H).

### Example 92: Synthesis of (2S)—N-[(1S)-1-cyano-2-[4-(1-ethyl-2-oxo-3H-indol-6-yl)phenyl]ethyl]-1,4-oxazepane-2-carboxamide (Compound 32)

### Step 1. Synthesis of 6-bromo-1-ethylindole-2,3-dione

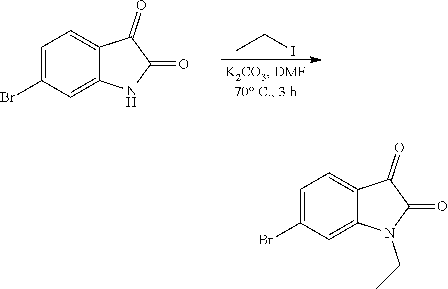

To a stirred solution of 6-bromo-1H-indole-2,3-dione (1 g, 4.424 mmol, 1.0 equiv) in DMF (15 mL) were added K₂CO₃ (1.22 g, 8.848 mmol, 2.0 equiv) and ethyl iodide (2.07 g, 13.272 mmol, 3.0 equiv). The resulting mixture was stirred for 3 h at 70° C. The mixture was allowed to cool down to room temperature. The resulting mixture was diluted with water (60 mL). The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE/THF (15:1) to afford 6-bromo-1-ethylindole-2,3-dione (850 mg, 75.6%) as an orange solid. LCMS (ES) [M+1]⁺ m/z: 254.

### Step 2. Synthesis of 6-bromo-1-ethyl-3H-indol-2-one

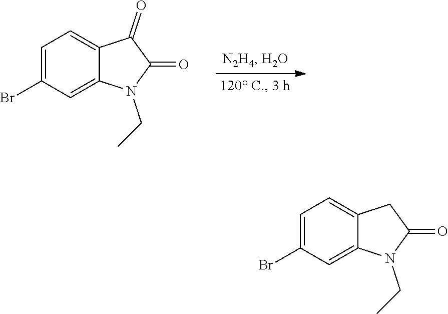

To a stirred solution of hydrazine hydrate (80%) (25 mL) was added 6-bromo-1-ethylindole-2,3-dione (850 mg, 3.345 mmol, 1.0 equiv). The resulting mixture was stirred for 3 h at 120° C. The mixture was allowed to cool down to room temperature. The resulting mixture was diluted with water (30 mL). The resulting mixture was extracted with EtOAc (3×15 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (9:1) to afford 6-bromo-1-ethyl-3H-indol-2-one (500 mg, 62.2%) as a white solid. LCMS (ES) [M+1]⁺ m/z: 240.

### Step 3. Synthesis of tert-butyl (2S)-2-{[(1S)-1-cyano-2-[4-(1-ethyl-2-oxo-3H-indol-6-yl)phenyl]ethyl]carbamoyl}-1,4-oxazepane-4-carboxylate

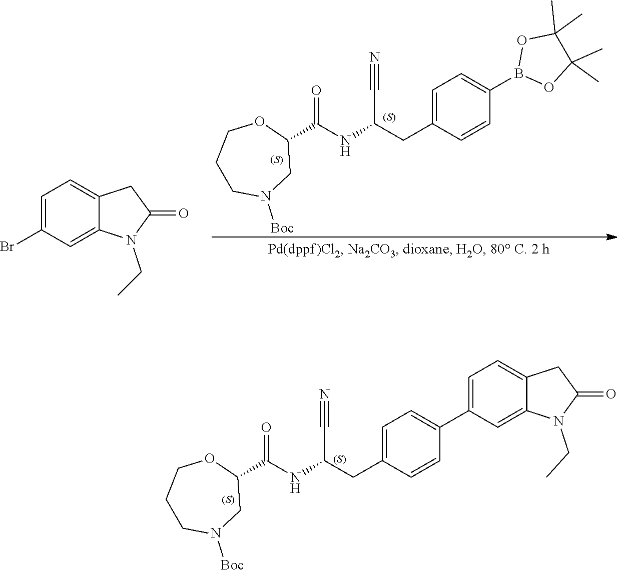

To a stirred solution of 6-bromo-1-ethyl-3H-indol-2-one (68 mg, 0.286 mmol, 1.1 equiv) and tert-butyl (2S)-2-{[(1S)-1-cyano-2-[4-(4,4,5,5-tetramethyl-1,3,2-dioxaborolan-2-yl)phenyl]ethyl]carbamoyl}-1,4-oxazepane-4-carboxylate (130 mg, 0.260 mmol, 1.0 equiv) in dioxane (2 mL) and H₂O (0.2 mL) were added Na₂CO₃ (55 mg, 0.520 mmol, 2.0 equiv) and Pd(dppf)Cl₂ (19 mg, 0.026 mmol, 0.1 equiv). The resulting mixture was stirred for 2 h at 80° C. under nitrogen atmosphere. The mixture was allowed to cool down to room temperature. The resulting mixture was concentrated under reduced pressure. The residue was purified by silica gel column chromatography, eluted with PE/THF (2:1) to afford tert-butyl (2S)-2-{[(1S)-1-cyano-2-[4-(1-ethyl-2-oxo-3H-indol-6-yl)phenyl]ethyl]carbamoyl}-1,4-oxazepane-4-carboxylate (100 mg, 72.1%) as a light yellow solid. LCMS (ES) [M+1]⁺ m/z: 533.

### Step 4. Synthesis of (2S)—N-[(1S)-1-cyano-2-[4-(1-ethyl-2-oxo-3H-indol-6-yl)phenyl]ethyl]-1,4-oxazepane-2-carboxamide

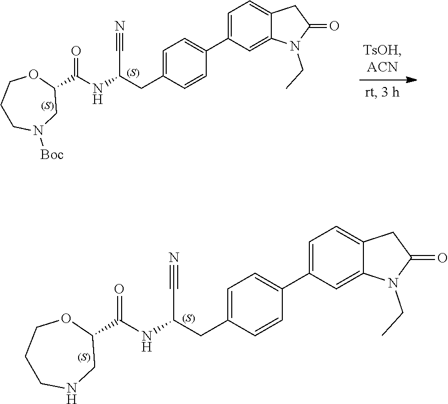

To a stirred solution of tert-butyl (2S)-2-{[(1S)-1-cyano-2-[4-(1-ethyl-2-oxo-3H-indol-6-yl)phenyl]ethyl]carbamoyl}-1,4-oxazepane-4-carboxylate (100 mg, 0.188 mmol, 1.0 equiv) in ACN (2 mL) was added TsOH (96 mg, 0.564 mmol, 3.0 equiv). The resulting mixture was stirred for 3 h at room temperature. The crude product was purified by Prep-HPLC with the following conditions (Column, XBridge Prep C18 OBD Column, 19*150 mm Sum; mobile phase, Water (0.1% NH₃H₂O) and ACN (10% PhaseB up to 80% in 20 min); Detector, UV 254 nm. This resulted in Compound 32 (30 mg, 36.9%) as a white solid. LCMS (ES) [M+1]⁺ m/z: 433. ¹H NMR (300 MHz, DMSO-d6) δ 8.62 (d, J=8.5 Hz, 1H), 7.67 (d, J=7.9 Hz, 2H), 7.43-7.24 (m, 5H), 5.03 (q, J=8.2 Hz, 1H), 4.00 (dd, J=7.9, 3.7 Hz, 1H), 3.91-3.65 (m, 4H), 3.58 (s, 2H), 3.28-3.11 (m, 2H), 3.03 (dd, J=14.3, 3.8 Hz, 1H), 2.84-2.69 (m, 1H), 2.67-2.52 (m, 2H), 1.87-1.60 (m, 2H), 1.18 (t, J=7.1 Hz, 3H).

### Example 93: Synthesis of (2S)—N-(1-cyano-2-{2,8-difluoro-6H-benzo[c]chromen-3-yl}ethyl)-1,4-oxazocane-2-carboxamide (Compound B16)

### Step 1. Synthesis of methyl 5-[(2-bromo-5-fluorophenyl)methoxy]-2-fluorobenzoate

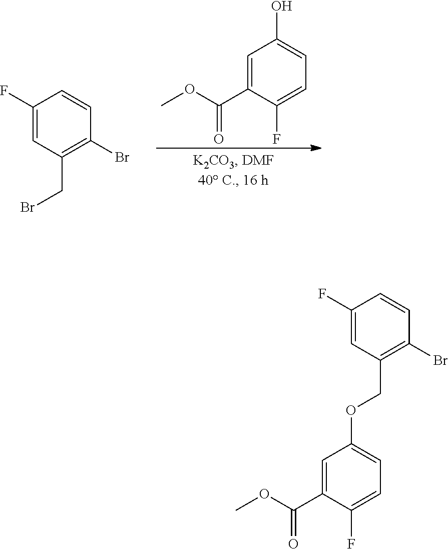 [SMILES: Fc1ccc(Br)c(CBr)c1 ; COC(=O)c1cc(O)ccc1F ; COC(=O)c1cc(OCc2cc(F)ccc2Br)ccc1F]

To a stirred solution of methyl 2-fluoro-5-hydroxybenzoate (5 g, 29.39 mmol, 1.0 equiv) and 1-bromo-2-(bromomethyl)-4-fluorobenzene (9.45 g, 35.27 mmol, 1.2 equiv) in DMF (90 mL) were added K₂CO₃ (6.09 g, 44.08 mmol, 1.5 equiv). The resulting mixture was stirred for 16 h at 40° C. The mixture was allowed to cool down to room temperature, diluted with water (200 mL), extracted with EtOAc (3×200 mL). The combined organic layer was washed with brine (2×200 mL), dried over anhydrous Na₂SO₄. After filtration, the filtrate was concentrated under reduced pressure. The residue was purified by silica gel column chromatography, eluted with PE/EA (12:1) to afford methyl 5-[(2-bromo-5-fluorophenyl)methoxy]-2-fluorobenzoate (9 g, 85.7%) as white solid. LCMS (ES): No LCMS signal.

### Step 2. Synthesis of methyl 2,8-difluoro-6H-benzo[c]chromene-3-carboxylate

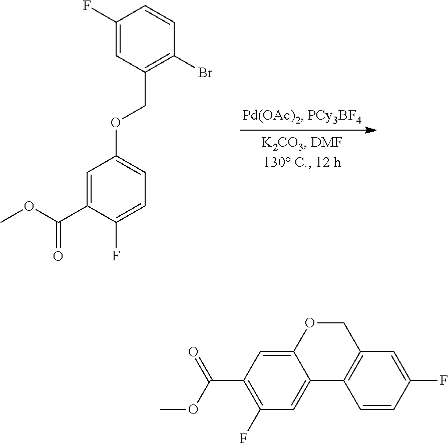 [SMILES: COC(=O)c1cc(OCc2cc(F)ccc2Br)ccc1F ; COC(=O)c1cc2c(cc1F)-c1ccc(F)cc1CO2]

To a stirred mixture of methyl 5-[(2-bromo-5-fluorophenyl)methoxy]-2-fluorobenzoate (7 g, 19.60 mmol, 1.0 equiv) and K₂CO₃ (5.42 g, 39.20 mmol, 2.0 equiv) in DMF (140 mL) were added PCy₃BF₄ (1.44 g, 3.92 mmol, 0.2 equiv) and Pd(OAc)₂ (0.44 g, 1.96 mmol, 0.1 equiv). The resulting mixture was stirred for 12 h at 130° C. under nitrogen atmosphere. The mixture was allowed to cool down to room temperature. The resulting mixture was diluted with water (200 mL), extracted with EtOAc (2×300 mL). The combined organic layer was washed with brine (2×300 mL), dried over anhydrous Na₂SO₄. After filtration, the filtrate was concentrated under reduced pressure. The residue was purified by silica gel column chromatography, eluted with PE/EA (12:1) to afford methyl 2,8-difluoro-6H-benzo[c]chromene-3-carboxylate (3 g, 55.4%) as white solid. LCMS (ES): No LCMS signal.

### Step 3. Synthesis of {2,8-difluoro-6H-benzo[c]chromen-3-yl}methanol

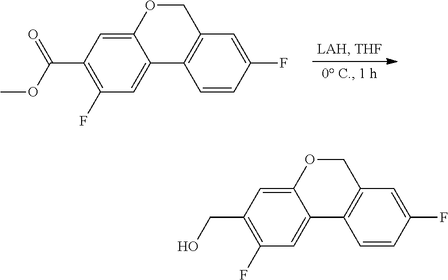 [SMILES: COC(=O)c1cc2c(cc1F)-c1ccc(F)cc1CO2 ; OCc1cc2c(cc1F)-c1ccc(F)cc1CO2]

To a stirred solution of methyl 2,8-difluoro-6H-benzo[c]chromene-3-carboxylate (3 g, 10.86 mmol, 1.0 equiv) in THF (30 mL) was added LAH (2 M in THF) (5.97 mL, 11.95 mmol, 1.1 equiv) dropwise at 0° C. under nitrogen atmosphere. The resulting mixture was stirred for 1 h at 0° C. under nitrogen atmosphere. The reaction was quenched by the addition of Na₂SO₄·10H₂O. The resulting mixture was filtered, the filter cake was washed with THF (3×10 mL). The filtrate was concentrated under reduced pressure. This resulted in {2,8-difluoro-6H-benzo[c]chromen-3-yl}methanol (2.4 g, 89%) as white solid. LCMS (ES) [M-H₂O+H]⁺ m/z: 231.

### Step 4. Synthesis of 3-(bromomethyl)-2,8-difluoro-6H-benzo[c]chromene

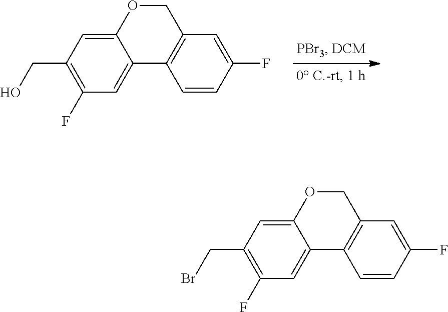 [SMILES: OCc1cc2c(cc1F)-c1ccc(F)cc1CO2 ; Fc1ccc2c(c1)COc1cc(CBr)c(F)cc1-2]

To a stirred solution of {2,8-difluoro-6H-benzo[c]chromen-3-yl}methanol (2.4 g, 9.67 mmol, 1.0 equiv) in DCM (30 mL) was added PBr₃ (1.31 g, 4.83 mmol, 0.5 equiv) dropwise at 0° C. under nitrogen atmosphere. The resulting mixture was stirred for 1 h at room temperature. The resulting mixture was diluted with water (30 mL). The resulting mixture was extracted with CH₂Cl₂ (2×30 mL). The combined organic layer was washed with brine (30 mL), dried over anhydrous Na₂SO₄. After filtration, the filtrate was concentrated under reduced pressure. This resulted in 3-(bromomethyl)-2,8-difluoro-6H-benzo[c]chromene (2.5 g, 83%) as white solid. LCMS (ES): No LCMS signal.

### Step 5. Synthesis of 3-{2,8-difluoro-6H-benzo[c]chromen-3-yl}-2-[(diphenylmethylidene)amino]propanenitrile

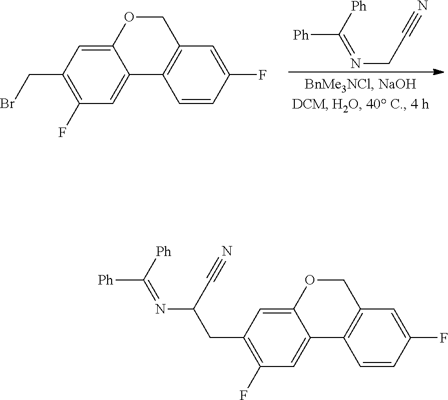 [SMILES: Fc1ccc2c(c1)COc1cc(CBr)c(F)cc1-2 ; N#CCN=C(c1ccccc1)c1ccccc1 ; N#CC(Cc1cc2c(cc1F)-c1ccc(F)cc1CO2)N=C(c1ccccc1)c1ccccc1]

To a stirred solution of 3-(bromomethyl)-2,8-difluoro-6H-benzo[c]chromene (2.5 g, 8.04 mmol, 1.0 equiv) and 2-[(diphenylmethylidene)amino]acetonitrile (1.77 g, 8.04 mmol, 1.0 equiv) in DCM (30 mL) and H₂O (3 mL) were added NaOH (0.64 g, 16.07 mmol, 2.0 equiv) and benzyltrimethylazanium chloride (0.15 g, 0.80 mmol, 0.1 equiv). The resulting mixture was stirred for 4 h at 40° C. The mixture was allowed to cool down to room temperature. The resulting mixture was diluted with water (30 mL). The resulting mixture was extracted with CH₂Cl₂ (3×30 mL). The combined organic layer was washed with brine (30 mL), dried over anhydrous Na₂SO₄. After filtration, the filtrate was concentrated under reduced pressure. The residue was purified by silica gel column chromatography, eluted with PE/THF (12:1) to afford 3-{2,8-difluoro-6H-benzo[c]chromen-3-yl}-2-[(diphenylmethylidene)amino]propanenitrile (2.5 g, 69%) as white solid. LCMS (ES) [M+H]⁺ m/z: 451.

### Step 6. Synthesis of 2-amino-3-{2,8-difluoro-6H-benzo[c]chromen-3-yl}propanenitrile

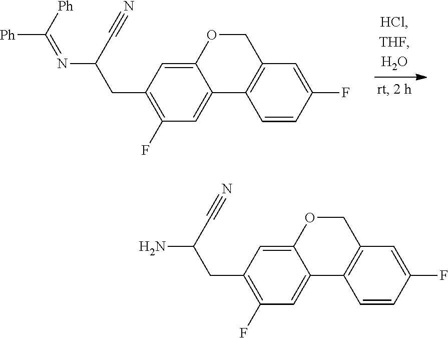 [SMILES: N#CC(Cc1cc2c(cc1F)-c1ccc(F)cc1CO2)N=C(c1ccccc1)c1ccccc1 ; N#CC(N)Cc1cc2c(cc1F)-c1ccc(F)cc1CO2]

To a stirred solution of 3-{2,8-difluoro-6H-benzo[c]chromen-3-yl}-2-[(diphenylmethylidene)amino]propanenitrile (2.5 g, 5.55 mmol, 1.0 equiv) in THF (110 mL) and H₂O (11 mL) were added HCl (1 N) (6.2 mL). The resulting mixture was stirred for 2 h at room temperature. The resulting mixture was diluted with water (100 mL). The aqueous layer was extracted with Et₂O (2×30 mL). The aqueous layer was basified to pH 12 with NaOH (3 N), extracted with CH₂Cl₂ (3×50 mL). The combined organic layer was washed with brine (30 mL), dried over anhydrous Na₂SO₄. After filtration, the filtrate was concentrated under reduced pressure. This resulted in 2-amino-3-{2,8-difluoro-6H-benzo[c]chromen-3-yl}propanenitrile (1.3 g, 81.8%) as light yellow solid. LCMS (ES) [M+H]⁺ m/z: 287.

### Step 7. Synthesis of tert-butyl (2S)-2-[(1-cyano-2-{2,8-difluoro-6H-benzo[c]chromen-3-yl}ethyl)carbamoyl]-1,4-oxazocane-4-carboxylate

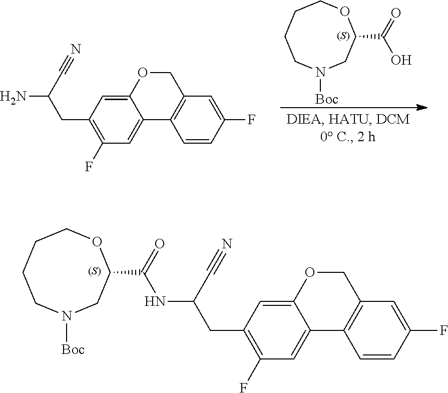

To a stirred solution of 2-amino-3-{2,8-difluoro-6H-benzo[c]chromen-3-yl}propanenitrile (79 mg, 0.28 mmol, 1.2 equiv) and (2S)-4-(tert-butoxycarbonyl)-1,4-oxazocane-2-carboxylic acid (60 mg, 0.23 mmol, 1.0 equiv) in DCM (1 mL) were added DIEA (89 mg, 0.69 mmol, 3.0 equiv) and HATU (105 mg, 0.28 mmol, 1.2 equiv) in portions at 0° C. under nitrogen atmosphere. The resulting mixture was stirred for 2 h at 0° C. under nitrogen atmosphere. The residue was purified by silica gel column chromatography, eluted with PE/THF (4:1) to afford tert-butyl (2S)-2-[(1-cyano-2-{2,8-difluoro-6H-benzo[c]chromen-3-yl}ethyl)carbamoyl]-1,4-oxazocane-4-carboxylate (100 mg, 82%) as colorless oil. LCMS (ES) [M+H]⁺ m/z: 528.

### Step 8. Synthesis of (2S)—N-(1-cyano-2-{2,8-difluoro-6H-benzo[c]chromen-3-yl}ethyl)-1,4-oxazocane-2-carboxamide

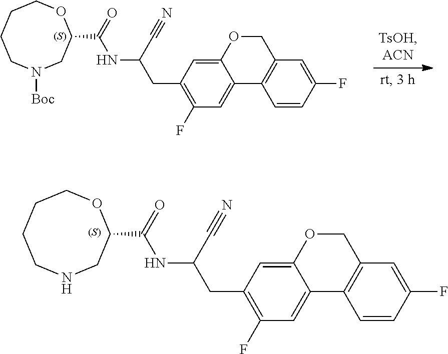

To a stirred solution of tert-butyl (2S)-2-[(1-cyano-2-{2,8-difluoro-6H-benzo[c]chromen-3-yl}ethyl)carbamoyl]-1,4-oxazocane-4-carboxylate (90 mg, 0.17 mmol, 1.0 equiv) in ACN (2 mL) was added TsOH (88 mg, 0.51 mmol, 3.0 equiv). The resulting mixture was stirred for 3 h at room temperature. The reaction solution was purified by Prep-HPLC with the following conditions: Column, XBridge Prep C18 OBD Column, 19*150 mm, 5 um; mobile phase, Water (0.1% NH₃H₂O) and ACN (10% Phase B up to 80% in 20 min); Detector, UV 254 nm. This resulted in Compound B16 (25 mg, 34.2%) as white solid. LCMS (ES) [M+H]⁺ m/z: 428. ¹H NMR (300 MHz, DMSO-d₆) δ 8.71 (dd, J=8.7, 3.4 Hz, 1H), 7.94-7.89 (m, 1H), 7.75 (d, J=10.6 Hz, 1H), 7.32-7.17 (m, 2H), 6.97 (t, J=6.5 Hz, 1H), 5.16-5.10 (m, 2H), 5.06-4.89 (m, 1H), 3.96 (t, J=9.9 Hz, 1H), 3.90-3.73 (m, 1H), 3.72-3.53 (m, 1H), 3.28-2.88 (m, 4H), 2.66-2.55 (m, 1H), 2.45-2.20 (m, 1H), 1.90-1.81 (m, 1H), 1.59-1.42 (m, 3H).

### Example 94: Synthesis of (2S,7R)—N-[(1S)-1-cyano-2-[2-fluoro-4-(1-methylindazol-6-yl)phenyl]ethyl]-7-methoxy-1,4-oxazocane-2-carboxamide (Compound H7)

### Step 1. Synthesis of tert-butyl (2S,7R)-2-{[(1S)-1-cyano-2-[2-fluoro-4-(1-methylindazol-6-yl)phenyl]ethyl]carbamoyl}-7-methoxy-1,4-oxazocane-4-carboxylate

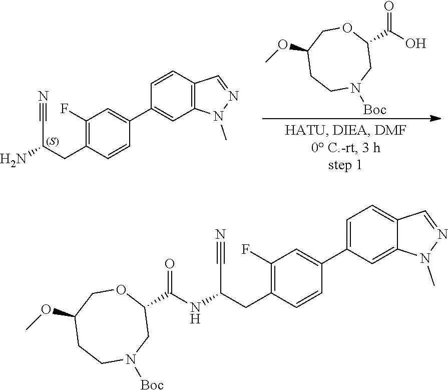

Into a 100 mL round-bottom flask were added (2S)-2-amino-3-[2-fluoro-4-(1-methylindazol-6-yl)phenyl]propanenitrile (915.57 mg, 3.11 mmol, 1.50 equiv), DMF (10.00 mL), (2S,7R)-4-(tert-butoxycarbonyl)-7-methoxy-1,4-oxazocane-2-carboxylic acid (600.00 mg, 2.07 mmol, 1.00 equiv) and DIEA (804.09 mg, 6.22 mmol, 3.00 equiv) at room temperature. To the above mixture was added HATU (946.22 mg, 2.48 mmol, 1.20 equiv) in portions at 0° C. The resulting mixture was stirred at 0° C. for additional 1 h. The reaction was quenched with ice water at 0° C. The resulting mixture was extracted with EtOAc (3×200 mL). The combined organic layers were dried over anhydrous Na₂SO₄. After filtration, the filtrate was concentrated under reduced pressure. The residue was purified by silica gel column chromatography, eluted with PE/EA (3:1) to afford tert-butyl (2S,7R)-2-{[(1S)-1-cyano-2-[2-fluoro-4-(1-methylindazol-6-yl)phenyl]ethyl]carbamoyl}-7-methoxy-1,4-oxazocane-4-carboxylate (700 mg, 59.68% yield, 85% purity) as colorless oil. LCMS (ES) [M+H]⁺ m/z: 566.

### Step 2. Synthesis of (2S,7R)—N-[(1S)-1-cyano-2-[2-fluoro-4-(1-methylindazol-6-yl)phenyl]ethyl]-7-methoxy-1,4-oxazocane-2-carboxamide

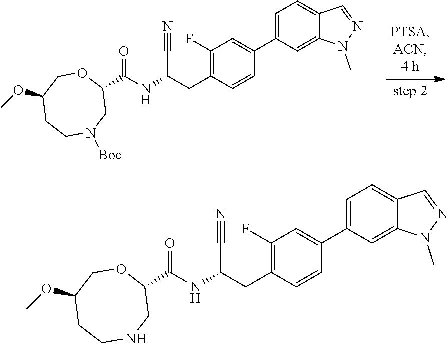 [SMILES: CO[C@@H]1CCN(C(=O)OC(C)(C)C)C[C@@H](C(=O)N[C@H](C#N)Cc2ccc(-c3ccc4cnn(C)c4c3)cc2F)OC1 ; CO[C@@H]1CCNC[C@@H](C(=O)N[C@H](C#N)Cc2ccc(-c3ccc4cnn(C)c4c3)cc2F)OC1]

Into a 40 mL vial were added tert-butyl (2S,7R)-2-{[(1S)-1-cyano-2-[2-fluoro-4-(1-methylindazol-6-yl)phenyl]ethyl]carbamoyl}-7-methoxy-1,4-oxazocane-4-carboxylate (650.00 mg, 1.14 mmol, 1.00 equiv) and MeCN (12.00 mL) at room temperature. To the above mixture was added TsOH (593.64 mg, 3.44 mmol, 3.00 equiv) in portions at 0° C. The resulting mixture was stirred at room temperature for additional 3 h. The residue was purified by reversed-phase flash chromatography with the following conditions: column, C18 silica gel; mobile phase, MeCN in Water (0.1% NH₃·H₂O), 30% to 65% gradient in 11 min; detector, UV 254 nm. This resulted in Compound H7 (200 mg, 37.39% yield, 98.77% purity) as white solid. LCMS (ES) [M+H]⁺ m/z: 466.3. ¹H NMR (300 MHz, DMSO-d6) δ 8.97-8.74 (m, 1H), 8.07 (d, J=0.9 Hz, 1H), 8.00 (s, 1H), 7.83 (d, J=8.5 Hz, 1H), 7.74-7.60 (m, 2H), 7.55-7.42 (m, 2H), 5.07 (q, J=8.3 Hz, 1H), 4.11 (s, 4H), 3.98-3.87 (m, 1H), 3.70-3.53 (m, 1H), 3.30 (d, J=23.7 Hz, 7H), 3.08-2.70 (m, 2H), 2.46-2.28 (m, 1H), 1.95-1.88 (m, 1H), 1.85-1.50 (m, 1H).

### Example 95: Synthesis of Compounds of the Disclosure

### Step 1. Synthesis

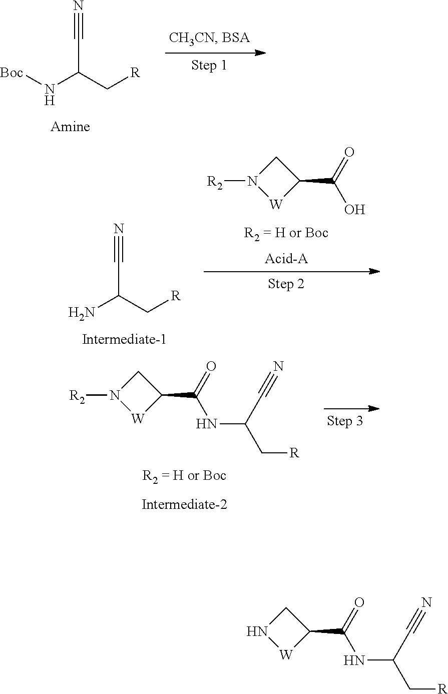

Step 1: Starting Amine (25-30 mg, 1.00 equiv), CH₃CN (2 mL) and N,O-bis(trimethylsilyl)acetamide (BSA) (1.20 equiv) were placed into a sealed 8-mL tube. The mixture was stirred for 4 h at 40° C. After cooled to room temperature. The reaction was used in the next step directly.

Examples of the starting Amine includes:

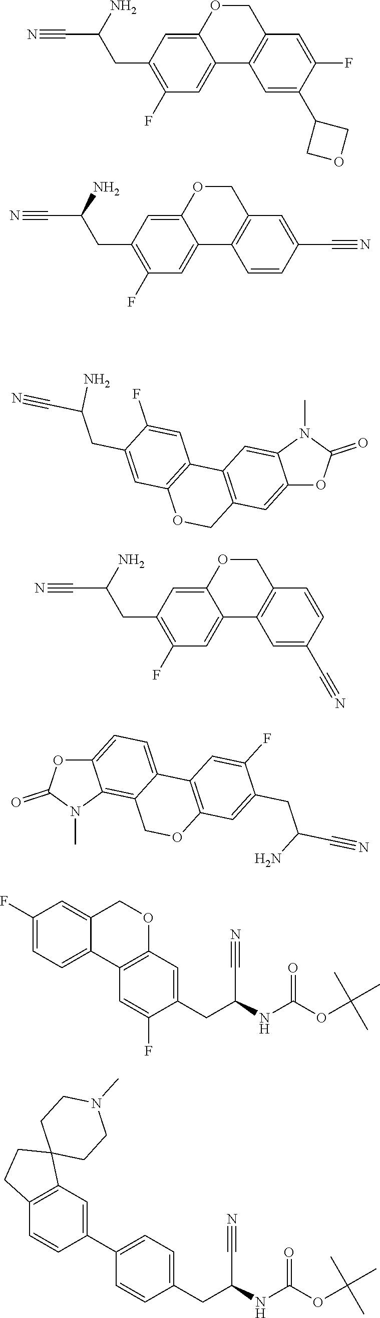

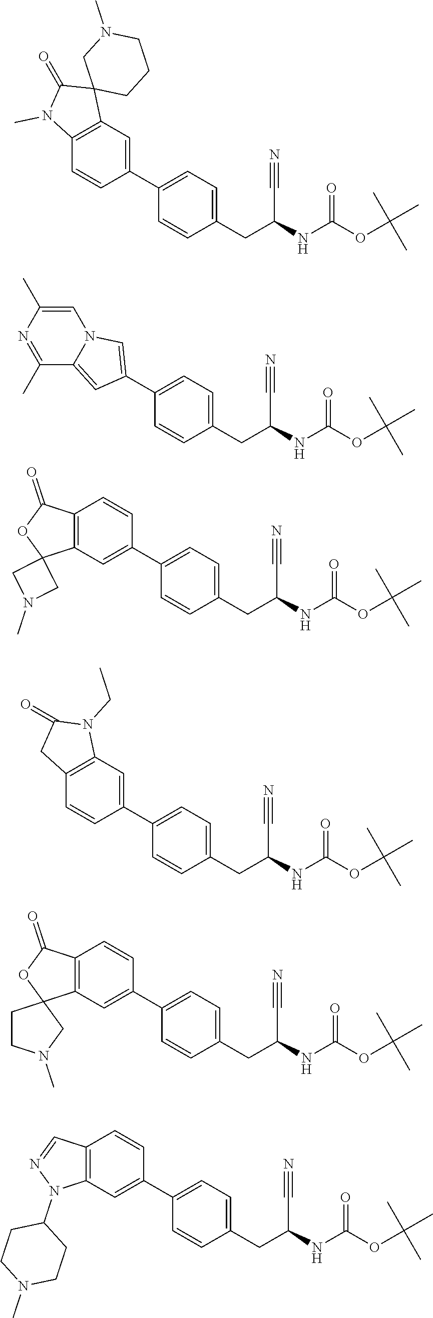 [SMILES: CN1CCCC2(C1)C(=O)N(C)c1ccc(-c3ccc(C[C@@H](C#N)NC(=O)OC(C)(C)C)cc3)cc12 ; Cc1cn2cc(-c3ccc(C[C@@H](C#N)NC(=O)OC(C)(C)C)cc3)cc2c(C)n1 ; CN1CC2(C1)OC(=O)c1ccc(-c3ccc(C[C@@H](C#N)NC(=O)OC(C)(C)C)cc3)cc12 ; CCN1C(=O)Cc2ccc(-c3ccc(C[C@@H](C#N)NC(=O)OC(C)(C)C)cc3)cc21 ; CN1CCC2(C1)OC(=O)c1ccc(-c3ccc(C[C@@H](C#N)NC(=O)OC(C)(C)C)cc3)cc12 ; CN1CCC(n2ncc3ccc(-c4ccc(C[C@@H](C#N)NC(=O)OC(C)(C)C)cc4)cc32)CC1]

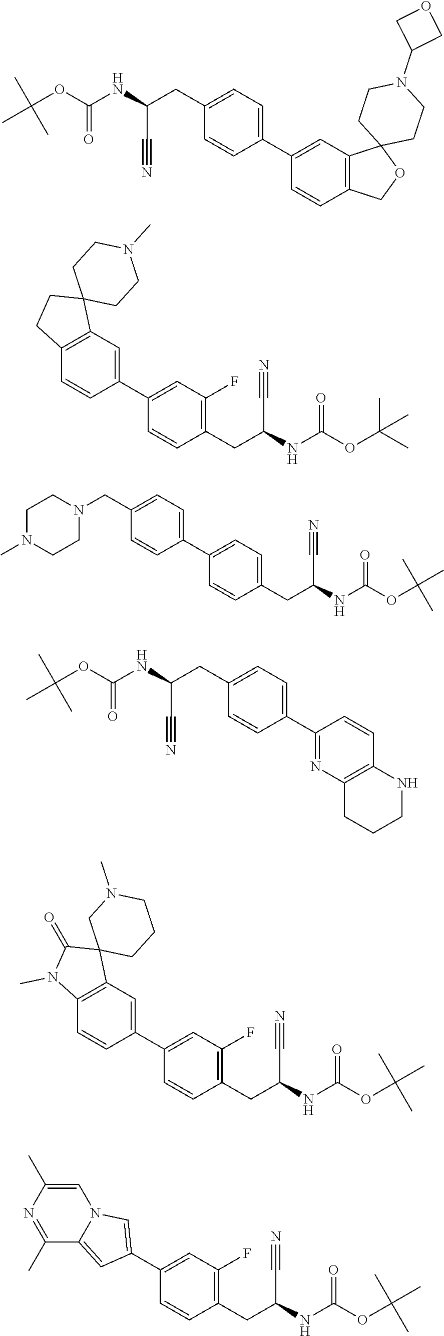

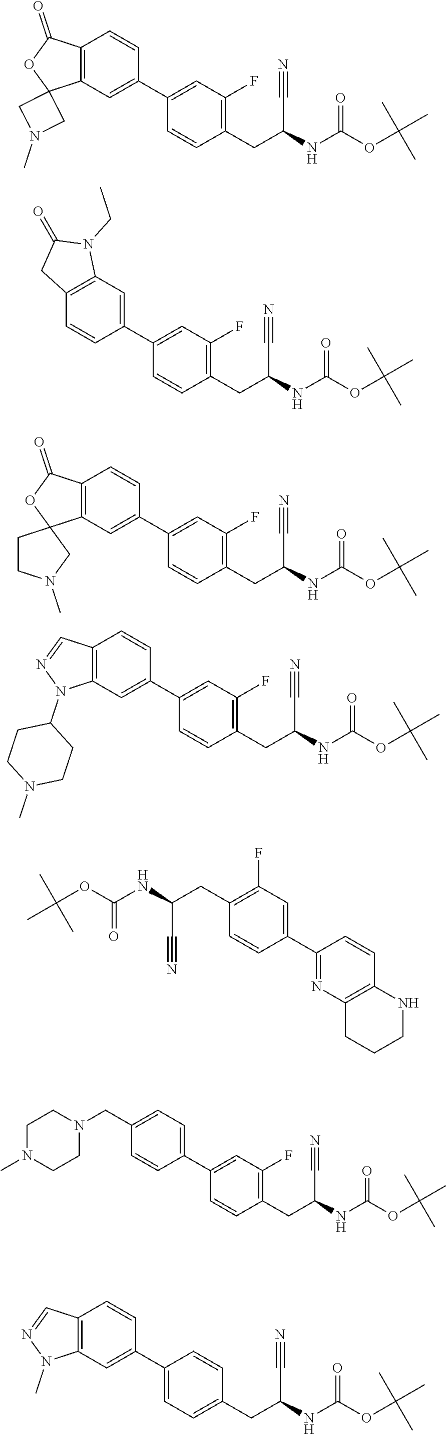

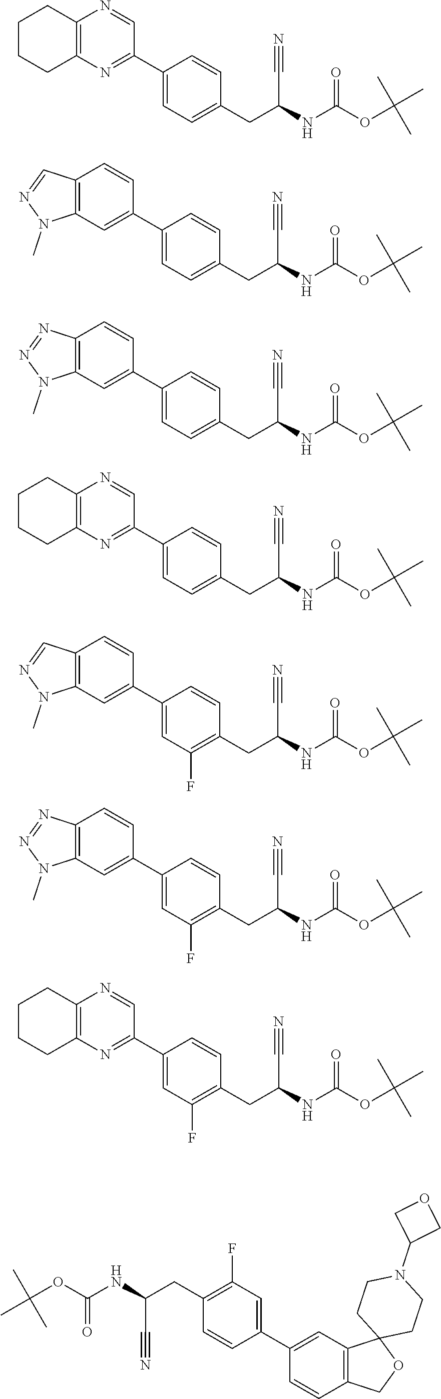

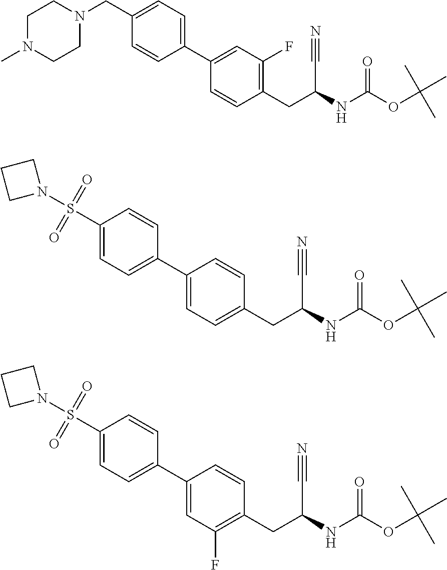 [SMILES: CN1CCN(Cc2ccc(-c3ccc(C[C@@H](C#N)NC(=O)OC(C)(C)C)c(F)c3)cc2)CC1 ; CC(C)(C)OC(=O)N[C@H](C#N)Cc1ccc(-c2ccc(S(=O)(=O)N3CCC3)cc2)cc1 ; CC(C)(C)OC(=O)N[C@H](C#N)Cc1ccc(-c2ccc(S(=O)(=O)N3CCC3)cc2)cc1F]

Step 2: Intermediate-1 (25-30 mg, 1.00 equiv) in CH₃CN, or the mixture of the preceding step were placed into a sealed 8-mL tube. Then DIEA (4.00 equiv), Acid-A (1.20 equiv), HATU (1.20 equiv) and CH₃CN (2 mL) were added to the tube. The mixture was stirred for 1 h at room temperature. The reaction was quenched with MeOH (1 mL). The mixture was purified by Prep-HPLC to give Intermediate-2.

Examples of Acid-A includes:

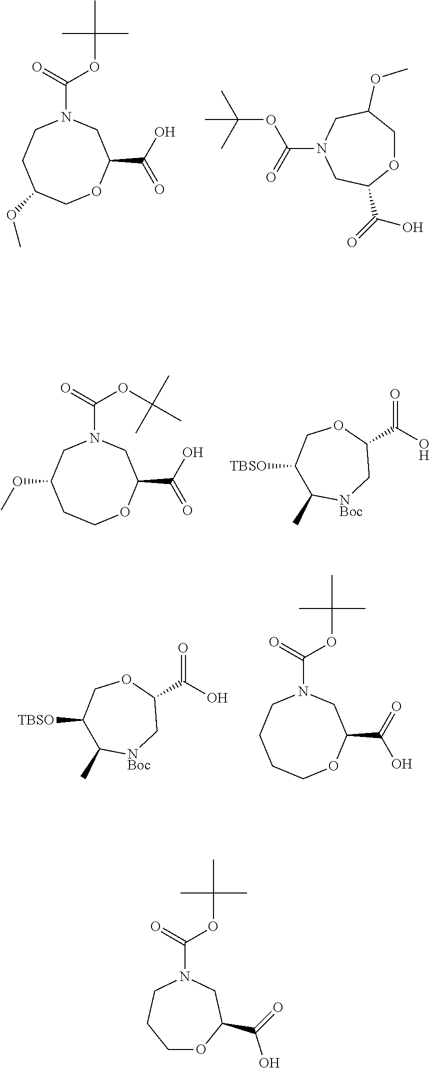

Step 3: Intermediate-2 (15-25 mg, 1.00 equiv), BSA (1.20 equiv) in CH₃CN (3 mL) was stirred at room temperature for 4 h. The reaction was quenched with MeOH (1 mL). The mixture was purified by Prep-HPLC to give the desired product.

Compounds prepared according to this Example are listed in Table 1 below.

TABLE 1
Synthesized Compounds

| Comp. No. | MW (g/mol) | Mass (ES, m/z) |
|---|---|---|
| A80 | 461.57 | [M + H]+ calcd 462.2, found 462.2 |
| A81 | 449.56 | [M + H]+ calcd 450.2, found 450.2 |
| A82 | 479.56 | [M + H]+ calcd 480.2, found 480.2 |
| A83 | 467.55 | [M + H]+ calcd 468.2, found 468.2 |
| A84 | 449.56 | [M + H]+ calcd 450.2, found 450.2 |
| A85 | 467.55 | [M + H]+ calcd 468.2, found 468.2 |
| A86 | 447.54 | [M + H]+ calcd 448.2, found 448.2 |
| A87 | 435.53 | [M + H]+ calcd 436.2, found 436.2 |
| A88 | 465.53 | [M + H]+ calcd 466.2, found 466.2 |
| A90 | 435.53 | [M + H]+ calcd 436.2, found 436.2 |
| A91 | 453.52 | [M + H]+ calcd 454.2, found 454.2 |
| A92 | 461.57 | [M + H]+ calcd 462.2, found 462.2 |
| A93 | 449.56 | [M + H]+ calcd 450.2, found 450.2 |
| A94 | 479.56 | [M + H]+ calcd 480.2, found 480.2 |
| A95 | 467.55 | [M + H]+ calcd 468.2, found 468.2 |
| A96 | 449.56 | [M + H]+ calcd 450.2, found 450.2 |
| A97 | 467.55 | [M + H]+ calcd 468.2, found 468.2 |
| A98 | 447.54 | [M + H]+ calcd 448.2, found 448.2 |
| A101 | 453.52 | [M + H]+ calcd 454.2, found 454.2 |
| A104 | 447.54 | [M + H]+ calcd 448.2, found 448.2 |
| A105 | 435.53 | [M + H]+ calcd 436.2, found 436.2 |
| A106 | 465.53 | [M + H]+ calcd 466.2, found 466.2 |
| A107 | 453.52 | [M + H]+ calcd 454.2, found 454.2 |
| A108 | 435.53 | [M + H]+ calcd 436.2, found 436.2 |
| A109 | 453.52 | [M + H]+ calcd 454.2, found 454.2 |
| A110 | 516.59 | [M + H]+ calcd 517.2, found 517.2 |
| A111 | 419.53 | [M + H]+ calcd 420.2, found 420.2 |
| A112 | 449.53 | [M + H]+ calcd 450.2, found 450.2 |
| A113 | 437.52 | [M + H]+ calcd 438.2, found 438.2 |
| A114 | 419.53 | [M + H]+ calcd 420.2, found 420.2 |
| A115 | 437.52 | [M + H]+ calcd 438.2, found 438.2 |
| A116 | 417.51 | [M + H]+ calcd 418.2, found 418.2 |
| A117 | 405.5 | [M + H]+ calcd 406.2, found 406.2 |
| A118 | 435.5 | [M + H]+ calcd 436.2, found 436.2 |
| A119 | 423.49 | [M + H]+ calcd 424.2, found 424.2 |
| A120 | 423.49 | [M + H]+ calcd 424.2, found 424.2 |
| A144 | 504.61 | [M + H]+ calcd 505.3, found 505.3 |
| B20 | 464.5 | [M + H]+ calcd 465.2, found 465.2 |
| B21 | 464.5 | [M + H]+ calcd 465.2, found 465.2 |
| B24 | 464.5 | [M + H]+ calcd 465.2, found 465.2 |
| B25 | 464.5 | [M + H]+ calcd 465.2, found 465.2 |
| B26 | 457.48 | [M + H]+ calcd 458.2, found 458.2 |
| B29 | 434.47 | [M + H]+ calcd 435.2, found 435.2 |
| B30 | 427.45 | [M + H]+ calcd 428.2, found 428.2 |
| B31 | 469.49 | [M + H]+ calcd 470.2, found 470.2 |
| B32 | 420.44 | [M + H]+ calcd 421.2, found 421.2 |
| B33 | 420.44 | [M + H]+ calcd 421.2, found 421.2 |
| B34 | 413.43 | [M + H]+ calcd 414.2, found 414.2 |
| C11 | 510.52 | [M + H]+ calcd 511.2, found 511.2 |
| C14 | 496.5 | [M + H]+ calcd 497.2, found 497.2 |
| C16 | 510.52 | [M + H]+ calcd 511.2, found 511.2 |
| C17 | 510.52 | [M + H]+ calcd 511.2, found 511.2 |
| C19 | 496.5 | [M + H]+ calcd 497.2, found 497.2 |
| C20 | 496.5 | [M + H]+ calcd 497.2, found 497.2 |
| C22 | 496.5 | [M + H]+ calcd 497.2, found 497.2 |
| C23 | 496.5 | [M + H]+ calcd 497.2, found 497.2 |
| C25 | 480.5 | [M + H]+ calcd 481.2, found 481.2 |
| C26 | 480.5 | [M + H]+ calcd 481.2, found 481.2 |
| C28 | 466.47 | [M + H]+ calcd 467.2, found 467.2 |
| C29 | 466.47 | [M + H]+ calcd 467.2, found 467.2 |
| F5 | 578.68 | [M + H]+ calcd 579.3, found 579.3 |
| F6 | 578.68 | [M + H]+ calcd 579.3, found 579.3 |
| F7 | 545.68 | [M + H]+ calcd 546.3, found 546.3 |
| F8 | 518.61 | [M + H]+ calcd 519.3, found 519.3 |
| F9 | 560.7 | [M + H]+ calcd 561.3, found 561.3 |
| F10 | 505.66 | [M + H]+ calcd 506.3, found 506.3 |
| F11 | 534.68 | [M + H]+ calcd 535.3, found 535.3 |
| F12 | 563.67 | [M + H]+ calcd 564.3, found 564.3 |
| F14 | 536.6 | [M + H]+ calcd 537.2, found 537.2 |
| F16 | 512.63 | [M + H]+ calcd 513.2, found 513.2 |
| F17 | 530.62 | [M + H]+ calcd 531.2, found 531.2 |
| F18 | 516.69 | [M + H]+ calcd 517.3, found 517.3 |
| F19 | 545.68 | [M + H]+ calcd 546.3, found 546.3 |
| F20 | 504.59 | [M + H]+ calcd 505.2, found 505.2 |
| F21 | 518.61 | [M + H]+ calcd 519.3, found 519.3 |
| F22 | 560.7 | [M + H]+ calcd 561.3, found 561.3 |
| F23 | 505.66 | [M + H]+ calcd 506.3, found 506.3 |
| F24 | 534.68 | [M + H]+ calcd 535.3, found 535.3 |
| F25 | 563.67 | [M + H]+ calcd 564.3, found 564.3 |
| F26 | 522.58 | [M + H]+ calcd 523.2, found 523.2 |
| F27 | 536.6 | [M + H]+ calcd 537.2, found 537.2 |
| F28 | 523.65 | [M + H]+ calcd 524.3, found 524.3 |
| F29 | 512.63 | [M + H]+ calcd 513.2, found 513.2 |
| F30 | 530.62 | [M + H]+ calcd 531.2, found 531.2 |
| G1 | 476.58 | [M + H]+ calcd 477.2, found 477.2 |
| G2 | 494.57 | [M + H]+ calcd 495.2, found 495.2 |
| G3 | 462.55 | [M + H]+ calcd 463.2, found 463.2 |
| G4 | 480.54 | [M + H]+ calcd 481.2, found 481.2 |
| G5 | 476.58 | [M + H]+ calcd 477.2, found 477.2 |
| G6 | 494.57 | [M + H]+ calcd 495.2, found 495.2 |
| G8 | 480.54 | [M + H]+ calcd 481.2, found 481.2 |
| G9 | 462.55 | [M + H]+ calcd 463.2, found 463.2 |
| G10 | 480.54 | [M + H]+ calcd 481.2, found 481.2 |
| G11 | 432.52 | [M + H]+ calcd 433.2, found 433.2 |
| G12 | 450.51 | [M + H]+ calcd 451.2, found 451.2 |
| H3 | 530.67 | [M + H]+ calcd 531.3, found 531.3 |
| H4 | 548.66 | [M + H]+ calcd 549.3, found 549.3 |
| H5 | 447.54 | [M + H]+ calcd 448.2, found 448.2 |
| H6 | 448.53 | [M + H]+ calcd 449.2, found 449.2 |
| H8 | 466.52 | [M + H]+ calcd 467.2, found 467.2 |
| H9 | 516.65 | [M + H]+ calcd 517.3, found 517.3 |
| H10 | 534.64 | [M + H]+ calcd 535.3, found 535.3 |
| H11 | 433.51 | [M + H]+ calcd 434.2, found 434.2 |
| H12 | 434.5 | [M + H]+ calcd 435.2, found 435.2 |
| H13 | 451.5 | [M + H]+ calcd 452.2, found 452.2 |
| H14 | 452.49 | [M + H]+ calcd 453.2, found 453.2 |
| H15 | 530.67 | [M + H]+ calcd 531.3, found 531.3 |
| H16 | 548.66 | [M + H]+ calcd 549.3, found 549.3 |
| H17 | 447.54 | [M + H]+ calcd 448.2, found 448.2 |
| H18 | 448.53 | [M + H]+ calcd 449.2, found 449.2 |
| H19 | 465.53 | [M + H]+ calcd 466.2, found 466.2 |
| H20 | 466.52 | [M + H]+ calcd 467.2, found 467.2 |
| H21 | 516.65 | [M + H]+ calcd 517.3, found 517.3 |
| H24 | 434.5 | [M + H]+ calcd 435.2, found 435.2 |
| H27 | 516.65 | [M + H]+ calcd 517.3, found 517.3 |
| H28 | 534.64 | [M + H]+ calcd 535.3, found 535.3 |
| H29 | 433.51 | [M + H]+ calcd 434.2, found 434.2 |
| H30 | 434.5 | [M + H]+ calcd 435.2, found 435.2 |
| H31 | 451.5 | [M + H]+ calcd 452.2, found 452.2 |
| H32 | 452.49 | [M + H]+ calcd 453.2, found 453.2 |
| H33 | 500.65 | [M + H]+ calcd 501.3, found 501.3 |
| H34 | 518.64 | [M + H]+ calcd 519.3, found 519.3 |
| H35 | 417.51 | [M + H]+ calcd 418.2, found 418.2 |
| H36 | 418.5 | [M + H]+ calcd 419.2, found 419.2 |
| H37 | 435.5 | [M + H]+ calcd 436.2, found 436.2 |
| H38 | 436.49 | [M + H]+ calcd 437.2, found 437.2 |

### Example 96: Mouse DPP1 Enzyme IC₅₀ Assay

Test articles were applied to active mouse DPP1 enzyme (R&D Systems; Minneapolis, MN) in Assay Buffer (50 mM MES pH 5.5, 50 mM NaCl, 5 mM DTT) in a total reaction volume of 125 μL. 25 μL of compound in Assay Buffer plus 500 DMSO was first added to 50 μL of active mouse DPP1 enzyme at a concentration of 62.5 pg/μL and allowed to pre-incubate for 10 minutes at 37° C. after which 50 μL of 1000 M H-Gly-Arg-AMC substrate (Bachem; St. Torrance, CA) was added, giving final substrate concentration of 400 M and a final DMSO concentration of 100. Substrate cleavage was measured for 90 minutes at 37° C., with fluorescence at Excitation/Emission 350/450 nm measured every 5 minutes. DPP1 concentration was interpolated based on its activity relative to a standard curve of recombinant active mouse DPP enzyme. IC₅₀ values for each compound were calculated via the XLFit (IDBS Version 5.3.1.3) Add-On to Microsoft Excel using the four parameter fit equation y=(A+((B−A)/(1+((C/x)\hat{ }D)))), which appears as equation number 205 (4 Parameter Logistic Model or Sigmoidal Dose-Response Model) in XLFit. Default constraints were used for each Parameter. IC₅₀ was defined as the compound concentration at which 50% of enzyme activity was inhibited when compared to the no-compound control.

Results are provided in Table 2 below. In Table 2, *** represent average IC₅₀≤5 nM, ** represents average IC₅₀ in the range of 5-20 nM, and * represents average IC₅₀ in the range of ≥20 nM, where the designations were made solely based on the average value without taking into account of the standard deviations.

TABLE 2
Mouse DPP1 enzyme IC₅₀ value ranges

| Comp. ID | Avg. IC₅₀ | |
|---|---|---|
| A1 | * | (n = 3) |
| A2 | * | (n = 1) |
| A3 | * | (n = 3) |
| A5 | * | (n = 1) |
| A8 | * | (n = 4) |
| A8 | * | (n = 2) |
| A10 | * | (n = 4) |
| A10 | * | (n = 2) |
| A11 | * | (n = 2) |
| A21 | * | (n = 3) |
| A27 | * | (n = 1) |
| A27 | * | (n = 2) |
| A28 | * | (n = 1) |
| A28 | * | (n = 2) |
| A47 | *** | (n = 1) |
| B2 | ** | (n = 3) |
| B7 | * | (n = 3) |
| B9 | ** | (n = 1) |
| B9 | ** | (n = 2) |
| B10-B | *** | (n = 3) |
| B16 | * | (n = 3) |
| F4 | *** | (n = 2) |
| 6 | * | (n = 1) |
| 7 | * | (n = 1) |
| 8 | * | (n = 2) |
| 9 | * | (n = 4) |
| 10 | * | (n = 2) |
| 11 | * | (n = 1) |
| 12 | * | (n = 1) |
| 14 | ** | (n = 1) |
| 18 | * | (n = 3) |
| 20 | * | (n = 3) |
| 21 | * | (n = 3) |
| 24 | ** | (n = 1) |
| 26 | * | (n = 3) |
| 28 | * | (n = 3) |
| 32 | * | (n = 3) |

### Example 97. Human DPP1 Enzyme IC₅₀ Assay

Recombinant human DPP1 enzyme (R&D Systems; Minneapolis, MN) was first proteolytically processed into its mature form using recombinant human cathepsin L (R&D Systems) in a buffer consisting of 20 mM citric acid pH 4.5, 150 mM NaCl, 1 mM EDTA and 10 mM DTT. Test articles were applied to activated human DPP1 enzyme in Assay Buffer (25 mM MES pH 6.0, 50 mM NaCl, 5 mM DTT) in a total reaction volume of 125 μL. 25 μL of compound in Assay Buffer plus 5% DMSO was first added to 50 μL of activated human DPP1 enzyme at a concentration of 1 ng/μL and allowed to pre-incubate for 10 minutes at 37° C. after which 50 μL of 1000 μM H-Gly-Arg-AMC substrate (Bachem; St. Torrance, CA) was added, giving final substrate concentration of 400 μM and a final DMSO concentration of 1%. Substrate cleavage was measured for 90 minutes at 37° C., with fluorescence at Excitation/Emission 350/450 nm measured every 5 minutes. DPP1 concentration was interpolated based on its activity relative to a standard curve of activated human recombinant DPP1 enzyme. IC50 values for each compound were calculated via the XLFit (IDBS Version 5.3.1.3) Add-On to Microsoft Excel using the four parameter fit equation y={A+[(B−A)]/[1+((C/x)\hat{ }D)]}, which appears as equation number 205 (4 Parameter Logistic Model or Sigmoidal Dose-Response Model) in XLFit. Default constraints were used for each Parameter. IC50 was defined as the compound concentration at which 50% of enzyme activity was inhibited when compared to the no-compound control.

Results are provided in Table 3 below. In Table 3, *** represent average IC₅₀≤5 nM, ** represents average IC₅₀ in the range of 5-15 nM, and * represents average IC₅₀ in the range of ≥15 nM, where the designations were made solely based on the average value without taking into account of the standard deviations.

TABLE 3
Human DPP1 enzyme IC₅₀ value ranges

| Comp. ID | Avg. IC₅₀ | |
|---|---|---|
| A1 | * | (n = 3) |
| A2 | * | (n = 3) |
| A3 | ** | (n = 3) |
| A4 | * | (n = 3) |
| A5 | * | (n = 3) |
| A6 | * | (n = 1) |
| A7 | * | (n = 1) |
| A8 | * | (n = 3) |
| A8 | * | (n = 1) |
| A9 | * | (n = 2) |
| A10 | * | (n = 3) |
| A10 | * | (n = 5) |
| A11 | * | (n = 3) |
| A12 | * | (n = 3) |
| A13 | * | (n = 1) |
| A15 | * | (n = 1) |
| A16 | * | (n = 1) |
| A17 | * | (n = 1) |
| A18 | * | (n = 3) |
| A19 | * | (n = 3) |
| A20 | * | (n = 2) |
| A21 | * | (n = 3) |
| A27 | * | (n = 3) |
| A27 | * | (n = 2) |
| A28 | * | (n = 3) |
| A28 | * | (n = 2) |
| A28 | * | (n = 1) |
| A30 | * | (n = 3) |
| A31 | * | (n = 1) |
| A32 | * | (n = 2) |
| A33 | *** | (n = 3) |
| A34 | * | (n = 2) |
| A35 | ** | (n = 2) |
| A36 | * | (n = 2) |
| A37 | * | (n = 1) |
| A38 | ** | (n = 4) |
| A39 | * | (n = 1) |
| A40 | * | (n = 3) |
| A41 | * | (n = 3) |
| A42 | * | (n = 2) |
| A43 | * | (n = 1) |
| A44 | * | (n = 1) |
| A45 | * | (n = 1) |
| A46 | * | (n = 1) |
| A47 | *** | (n = 1) |
| A47 | *** | (n = 4) |
| A48 | ** | (n = 1) |
| A49 | ** | (n = 1) |
| A50 | *** | (n = 2) |
| A51 | ** | (n = 1) |
| A52 | * | (n = 1) |
| A54 | * | (n = 4) |
| A57 | * | (n = 1) |
| A60 | * | (n = 1) |
| A63 | * | (n = 1) |
| A66 | * | (n = 2) |
| A88 | *** | (n = 2) |
| A88 | *** | (n = 2) |
| A88-A | * | (n = 1) |
| A88-B | *** | (n = 1) |
| A92 | * | (n = 1) |
| A92 | * | (n = 1) |
| A105 | * | (n = 1) |
| A105 | * | (n = 1) |
| A105 | * | (n = 1) |
| A109 | ** | (n = 1) |
| A109 | *** | (n = 1) |
| A109 | ** | (n = 2) |
| A137 | * | (n = 1) |
| A138 | * | (n = 1) |
| A142 | * | (n = 1) |
| A143 | * | (n = 1) |
| A145 | *** | (n = 1) |
| A146 | *** | (n = 1) |
| A147 | ** | (n = 1) |
| A148 | * | (n = 1) |
| A149 | * | (n = 1) |
| A151 | *** | (n = 1) |
| A152 | *** | (n = 1) |
| A153 | *** | (n = 1) |
| A154 | ** | (n = 1) |
| A155 | ** | (n = 1) |
| A158 | *** | (n = 1) |
| A160 | ** | (n = 1) |
| A161 | * | (n = 1) |
| A164 | *** | (n = 1) |
| A165 | ** | (n = 1) |
| A166 | ** | (n = 1) |
| A169 | *** | (n = 2) |
| A170 | ** | (n = 2) |
| A171 | * | (n = 1) |
| A172 | * | (n = 2) |
| B1 | * | (n = 1) |
| B2 | ** | (n = 3) |
| B3 | * | (n = 1) |
| B4 | * | (n = 3) |
| B7 | * | (n = 3) |
| B8 | * | (n = 1) |
| B9 | ** | (n = 3) |
| B9 | * | (n = 5) |
| B10 | ** | (n = 3) |
| B10-A | * | (n = 2) |
| B10-B | ** | (n = 4) |
| B11 | *** | (n = 3) |
| B12 | *** | (n = 2) |
| B13 | *** | (n = 2) |
| B14 | * | (n = 1) |
| B15 | *** | (n = 1) |
| B16 | * | (n = 3) |
| B17 | ** | (n = 1) |
| B32 | ** | (n = 2) |
| B33 | * | (n = 2) |
| B35 | * | (n = 1) |
| C1 | *** | (n = 2) |
| C1-A | * | (n = 1) |
| C1-B | *** | (n = 1) |
| C2 | * | (n = 3) |
| C2-A | * | (n = 1) |
| C2-B | * | (n = 1) |
| C3 | *** | (n = 1) |
| C4 | * | (n = 1) |
| C5 | *** | (n = 2) |
| C6 | * | (n = 2) |
| C8 | *** | (n = 2) |
| C10 | * | (n = 2) |
| C29 | * | (n = 2) |
| C29 | * | (n = 1) |
| C31 | *** | (n = 2) |
| C32 | *** | (n = 2) |
| C33 | *** | (n = 2) |
| C34 | *** | (n = 2) |
| D1 | * | (n = 1) |
| D2 | * | (n = 1) |
| D4 | * | (n = 3) |
| D7 | * | (n = 1) |
| D8 | * | (n = 3) |
| D9 | * | (n = 2) |
| E1 | *** | (n = 2) |
| E6 | *** | (n = 2) |
| F1 | * | (n = 2) |
| F2 | * | (n = 2) |
| F3 | * | (n = 1) |
| F4 | *** | (n = 1) |
| F4 | *** | (n = 3) |
| F5 | *** | (n = 1) |
| F6 | *** | (n = 1) |
| G2 | *** | (n = 1) |
| G2 | *** | (n = 1) |
| G9 | * | (n = 1) |
| G9 | * | (n = 1) |
| G10 | ** | (n = 1) |
| G10 | ** | (n = 1) |
| G11 | * | (n = 2) |
| G12 | ** | (n = 1) |
| G12 | ** | (n = 2) |
| H1 | *** | (n = 1) |
| H2 | ** | (n = 2) |
| H5 | ** | (n = 1) |
| H5 | ** | (n = 2) |
| H7 | *** | (n = 2) |
| H12 | *** | (n = 2) |
| H12 | *** | (n = 1) |
| H12 | *** | (n = 2) |
| H12-A | * | (n = 1) |
| H12-B | *** | (n = 1) |
| H29 | * | (n = 1) |
| H29 | * | (n = 1) |
| H29 | * | (n = 1) |
| H31 | ** | (n = 1) |
| H31 | * | (n = 1) |
| H31 | ** | (n = 2) |
| I2 | ** | (n = 1) |
| I3 | ** | (n = 1) |
| I5 | ** | (n = 1) |
| I6 | ** | (n = 1) |
| I7 | * | (n = 2) |
| I8 | ** | (n = 1) |
| I9 | ** | (n = 2) |
| I12 | ** | (n = 2) |
| I13 | * | (n = 2) |
| I14 | * | (n = 2) |
| I15 | * | (n = 2) |
| I16 | * | (n = 2) |
| I18 | * | (n = 2) |
| I20 | * | (n = 2) |
| 1 | * | (n = 1) |
| 2 | * | (n = 1) |
| 3 | * | (n = 1) |
| 4 | * | (n = 1) |
| 6 | * | (n = 1) |
| 7 | * | (n = 3) |
| 8 | * | (n = 3) |
| 9 | * | (n = 3) |
| 10 | * | (n = 3) |
| 11 | * | (n = 1) |
| 12 | * | (n = 3) |
| 13 | * | (n = 4) |
| 14 | ** | (n = 1) |
| 18 | * | (n = 3) |
| 19 | * | (n = 3) |
| 20 | * | (n = 3) |
| 21 | * | (n = 3) |
| 22 | ** | (n = 1) |
| 23 | ** | (n = 1) |
| 24 | ** | (n = 1) |
| 25 | * | (n = 1) |
| 26 | * | (n = 3) |
| 27 | * | (n = 1) |
| 28 | * | (n = 3) |
| 29 | * | (n = 1) |
| 30 | ** | (n = 1) |
| 31 | * | (n = 2) |
| 32 | * | (n = 3) |
| 32 | * | (n = 5) |
| 35 | * | (n = 2) |

### Example 98. Human DPP1 Enzyme Single-Point Percent Inhibition Testing

A crude lysate of HL-60 cells (ATCC; Manassas, VA) was used as a source of human DPP1 enzyme in the assay. Lysate was prepared in 1% Triton X-100 in PBS at a concentration of 20,000 live cells per μL of lysis buffer and was centrifuged at 16,000 rcf for 10 minutes at 4° C., after which supernatant was collected and flash-frozen in liquid nitrogen. Test articles were applied to human DPP1 enzyme in Assay Buffer (25 mM MES pH 6.0, 50 mM NaCl, 5 mM DTT) in a total reaction volume of 125 μL. 25 μL of compound diluted to 50 nM in Assay Buffer plus 5% DMSO was first added to 50 μL of HL-60 lysate diluted to contain a DPP1 enzyme concentration of roughly 1 ng/μL and allowed to pre-incubate for 10 minutes at 37° C. after which 50 μL of 1000 μM H-Gly-Arg-AMC substrate (Bachem; St. Torrance, CA) was added, giving final compound concentration of 10 nM, a final substrate concentration of 400 μM and a final DMSO concentration of 1%. Substrate cleavage was measured for 90 minutes at 37° C., with fluorescence at Excitation/Emission 350/450 nm measured every 5 minutes. DPP1 concentration was interpolated based on its activity relative to a standard curve of activated human recombinant DPP1 enzyme. Percent inhibition at 10 nM was calculated for each test article based on remaining DPP1 activity compared to enzyme activity in wells that received only vehicle control.

Results are provided in Table 4 below. In Table 4, *** represents percent inhibition of ≥66.7%, ** represents percent inhibition in the range of 40-66.7%, and * represents percent inhibition of ≤40%.

TABLE 4
Human DPP1 Enzyme Single-Point Percent Inhibition (n = 1)

| Comp. ID | % Inhibition |
|---|---|
| 18 | * |
| 19 | * |
| 36 | * |
| 37 | * |
| 38 | * |
| 39 | * |
| 40 | * |
| 41 | ** |
| 42 | ** |
| 43 | ** |
| 44 | ** |
| 45 | * |
| 46 | * |
| 47 | * |
| 48 | ** |
| 49 | * |
| A11 | * |
| A80 | *** |
| A81 | ** |
| A82 | *** |
| A83 | *** |
| A84 | ** |
| A85 | *** |
| A86 | ** |
| A87 | *** |
| A88 | *** |
| A90 | *** |
| A91 | *** |
| A92 | * |
| A93 | * |
| A94 | * |
| A95 | ** |
| A96 | ** |
| A97 | ** |
| A98 | *** |
| A101 | *** |
| A104 | * |
| A105 | * |
| A106 | * |
| A107 | * |
| A108 | * |
| A109 | ** |
| A110 | * |
| A111 | * |
| A112 | * |
| A113 | * |
| A114 | * |
| A115 | * |
| A116 | * |
| A117 | * |
| A118 | * |
| A119 | * |
| A120 | * |
| A122 | *** |
| A123 | *** |
| A125 | *** |
| A126 | *** |
| A128 | ** |
| A129 | ** |
| A131 | *** |
| A132 | *** |
| A134 | ** |
| A135 | * |
| A137 | * |
| A138 | * |
| A140 | * |
| A141 | * |
| A142 | * |
| A144 | ** |
| B17 | ** |
| B20 | *** |
| B21 | *** |
| B24 | ** |
| B25 | ** |
| B26 | ** |
| B28 | ** |
| B29 | * |
| B30 | * |
| B31 | ** |
| B32 | ** |
| B33 | * |
| B34 | ** |
| C8 | *** |
| C11 | *** |
| C14 | *** |
| C16 | *** |
| C17 | ** |
| C19 | *** |
| C20 | *** |
| C22 | *** |
| C23 | ** |
| C25 | *** |
| C26 | * |
| C28 | *** |
| C29 | * |
| C31 | *** |
| C32 | *** |
| F5 | *** |
| F6 | *** |
| F7 | *** |
| F8 | *** |
| F9 | *** |
| F10 | *** |
| F11 | *** |
| F12 | *** |
| F14 | *** |
| F15 | *** |
| F16 | *** |
| F17 | *** |
| F18 | *** |
| F19 | ** |
| F20 | ** |
| F21 | ** |
| F22 | ** |
| F23 | * |
| F24 | *** |
| F25 | *** |
| F26 | *** |
| F27 | *** |
| F28 | * |
| F29 | * |
| F30 | ** |
| G1 | *** |
| G2 | *** |
| G3 | *** |
| G4 | *** |
| G5 | ** |
| G6 | *** |
| G8 | *** |
| G9 | * |
| G10 | ** |
| G11 | * |
| G12 | ** |
| H3 | *** |
| H4 | *** |
| H5 | *** |
| H6 | *** |
| H8 | *** |
| H9 | *** |
| H10 | *** |
| H11 | *** |
| H12 | *** |
| H13 | *** |
| H14 | *** |
| H15 | ** |
| H16 | *** |
| H17 | * |
| H18 | * |
| H19 | *** |
| H20 | ** |
| H21 | *** |
| H24 | *** |
| H27 | * |
| H28 | *** |
| H29 | * |
| H30 | * |
| H31 | ** |
| H32 | ** |
| H33 | * |
| H34 | *** |
| H35 | * |
| H36 | * |
| H37 | ** |
| H38 | * |
| H39 | ** |
| H40 | ** |
| H41 | * |
| H42 | *** |
| H43 | * |
| H44 | * |
| I2 | ** |
| I3 | *** |
| I8 | *** |

### Example 99. DPP1 Cell IC₅₀ assay

HL-60 cells (ATCC; Manassas, VA) were maintained in RPMI-1640 supplemented with 20% heat-inactivated FBS and 1× Antibiotic Antimycotic (Cytiva; Marlborough, MA). Media was changed every three to four days and cells were not allowed to exceed 1×10⁶ cells per mL. Prior to assay, cells were collected by centrifugation at 500 rcf for 3 minutes, resuspended in PBS and counted. Cells were diluted in PBS to a concentration of 5×10⁵ live cells per mL and transferred to black 96-well plates for assay, 60 μL per well. Test articles were diluted in PBS plus 0.5% DMSO, and 20 μL was added to each assay well. Compound was allowed to pre-incubate with cells with gentle shaking at 100 rpm for 60 minutes at 37° C. in a cell culture incubator maintained at 5% CO₂, after which 20 μL of 500 μM H-Gly-Phe-AFC substrate (MP Biomedicals; Solon, OH) was added to each well. Plates were returned to the incubator with shaking at 100 rpm for 30 minutes, after which fluorescence was measured at Excitation/Emission 400/505 nm. % Inhibition was calculated from RFU values compared to control cell wells that received only PBS plus 0.5% DMSO. IC₅₀ values for each compound were calculated via the XLFit (IDBS Version 5.3.1.3) Add-On to Microsoft Excel using the four parameter fit equation y=(A+((B−A)/(1+((C/x)\hat{ }D)))), which appears as equation number 205 (4 Parameter Logistic Model or Sigmoidal Dose-Response Model) in XLFit. IC₅₀ was defined as the compound concentration at which 50% of enzyme activity was inhibited when compared to the no-compound control.

The result of the assay is provided in Table 5, below. In Table 5, *** represent average IC₅₀≤1 nM, ** represents average IC₅₀ in the range of 1-2.5 nM, and * represents average IC₅₀ in the range of ≥2.5 nM, where the designations were made solely based on the average value without taking into account of the standard deviations.

TABLE 5
DPP1 Cell IC₅₀ value ranges

| Comp. ID | Avg. IC₅₀ | |
|---|---|---|
| A1 | * | (n = 3) |
| A1 | * | (n = 2) |
| A2 | * | (n = 2) |
| A3 | * | (n = 3) |
| A4 | * | (n = 3) |
| A5 | * | (n = 3) |
| A6 | * | (n = 1) |
| A7 | * | (n = 1) |
| A8 | * | (n = 3) |
| A8 | * | (n = 3) |
| A9 | * | (n = 1) |
| A10 | * | (n = 3) |
| A10 | * | (n = 3) |
| A11 | * | (n = 3) |
| A12 | * | (n = 1) |
| A18 | * | (n = 1) |
| A19 | * | (n = 1) |
| A20 | * | (n = 1) |
| A21 | * | (n = 3) |
| A27 | * | (n = 2) |
| A27 | ** | (n = 2) |
| A28 | ** | (n = 2) |
| A28 | ** | (n = 2) |
| A28 | ** | (n = 1) |
| A30 | * | (n = 1) |
| A31 | * | (n = 1) |
| A32 | ** | (n = 2) |
| A33 | * | (n = 2) |
| A34 | ** | (n = 2) |
| A35 | ** | (n = 2) |
| A36 | * | (n = 1) |
| A37 | * | (n = 1) |
| A38 | ** | (n = 3) |
| A39 | * | (n = 1) |
| A40 | * | (n = 1) |
| A41 | * | (n = 2) |
| A42 | ** | (n = 2) |
| A43 | * | (n = 2) |
| A44 | * | (n = 1) |
| A45 | * | (n = 1) |
| A46 | * | (n = 1) |
| A47 | ** | (n = 2) |
| A47 | ** | (n = 3) |
| A48 | ** | (n = 2) |
| A49 | * | (n = 2) |
| A50 | ** | (n = 2) |
| A51 | ** | (n = 2) |
| A52 | * | (n = 2) |
| A54 | * | (n = 3) |
| A57 | * | (n = 1) |
| A60 | * | (n = 1) |
| A63 | * | (n = 1) |
| A66 | * | (n = 2) |
| A88 | ** | (n = 1) |
| A88 | ** | (n = 1) |
| A88-A | * | (n = 1) |
| A88-B | *** | (n = 2) |
| A92 | * | (n = 1) |
| A92 | ** | (n = 1) |
| A92 | ** | (n = 1) |
| A105 | * | (n = 1) |
| A105 | * | (n = 1) |
| A105 | * | (n = 1) |
| A109 | * | (n = 1) |
| A109 | * | (n = 1) |
| A109 | ** | (n = 3) |
| A137 | ** | (n = 1) |
| A138 | *** | (n = 1) |
| A142 | * | (n = 1) |
| A143 | * | (n = 1) |
| A145 | ** | (n = 1) |
| A146 | *** | (n = 1) |
| A147 | ** | (n = 1) |
| A148 | * | (n = 1) |
| A149 | ** | (n = 1) |
| A151 | ** | (n = 1) |
| A152 | *** | (n = 1) |
| A153 | *** | (n = 1) |
| A154 | ** | (n = 1) |
| A155 | ** | (n = 1) |
| A158 | *** | (n = 1) |
| A160 | ** | (n = 1) |
| A161 | * | (n = 1) |
| A164 | *** | (n = 1) |
| A165 | ** | (n = 1) |
| A166 | ** | (n = 1) |
| A169 | *** | (n = 1) |
| A171 | * | (n = 1) |
| B1 | * | (n = 1) |
| B2 | ** | (n = 3) |
| B4 | * | (n = 2) |
| B7 | * | (n = 3) |
| B9 | ** | (n = 3) |
| B9 | ** | (n = 3) |
| B10 | ** | (n = 2) |
| B10-A | * | (n = 1) |
| B10-B | ** | (n = 3) |
| B11 | ** | (n = 2) |
| B12 | ** | (n = 2) |
| B13 | *** | (n = 2) |
| B14 | * | (n = 1) |
| B15 | ** | (n = 2) |
| B16 | * | (n = 3) |
| B17 | * | (n = 1) |
| B32 | ** | (n = 1) |
| B33 | * | (n = 1) |
| B35 | * | (n = 1) |
| C1 | * | (n = 2) |
| C1-A | * | (n = 2) |
| C1-B | ** | (n = 2) |
| C2 | * | (n = 2) |
| C2-A | ** | (n = 2) |
| C3 | * | (n = 1) |
| C5 | ** | (n = 2) |
| C6 | * | (n = 2) |
| C8 | ** | (n = 1) |
| C10 | ** | (n = 1) |
| C29 | * | (n = 1) |
| C29 | ** | (n = 3) |
| C31 | * | (n = 1) |
| C32 | * | (n = 1) |
| C33 | ** | (n = 1) |
| C34 | ** | (n = 1) |
| D1 | * | (n = 1) |
| D2 | * | (n = 1) |
| D4 | * | (n = 2) |
| D7 | * | (n = 1) |
| D8 | * | (n = 1) |
| D9 | * | (n = 1) |
| E1 | ** | (n = 1) |
| E6 | * | (n = 2) |
| F1 | * | (n = 2) |
| F2 | * | (n = 2) |
| F3 | * | (n = 2) |
| F4 | *** | (n = 3) |
| F4 | *** | (n = 3) |
| F5 | ** | (n = 2) |
| F6 | * | (n = 1) |
| G2 | ** | (n = 1) |
| G2 | ** | (n = 3) |
| G9 | * | (n = 3) |
| G9 | * | (n = 1) |
| G10 | * | (n = 1) |
| G10 | * | (n = 3) |
| G11 | * | (n = 1) |
| G12 | * | (n = 1) |
| G12 | ** | (n = 3) |
| H5 | ** | (n = 1) |
| H5 | ** | (n = 3) |
| H7 | *** | (n = 3) |
| H12 | * | (n = 1) |
| H12 | ** | (n = 1) |
| H12 | ** | (n = 3) |
| H12-A | * | (n = 1) |
| H12-B | ** | (n = 3) |
| H29 | * | (n = 1) |
| H29 | * | (n = 1) |
| H29 | * | (n = 1) |
| H31 | ** | (n = 1) |
| H31 | ** | (n = 3) |
| H31 | ** | (n = 3) |
| I2 | * | (n = 1) |
| I3 | * | (n = 1) |
| I5 | * | (n = 1) |
| I6 | * | (n = 1) |
| I7 | * | (n = 1) |
| I8 | * | (n = 1) |
| I9 | * | (n = 1) |
| I12 | * | (n = 1) |
| I13 | * | (n = 1) |
| I14 | * | (n = 1) |
| I15 | * | (n = 1) |
| I16 | * | (n = 1) |
| I18 | * | (n = 1) |
| I20 | * | (n = 1) |
| 4 | * | (n = 1) |
| 6 | * | (n = 1) |
| 7 | * | (n = 3) |
| 8 | * | (n = 3) |
| 9 | * | (n = 3) |
| 10 | * | (n = 3) |
| 11 | * | (n = 1) |
| 12 | * | (n = 3) |
| 13 | * | (n = 1) |
| 13 | * | (n = 1) |
| 14 | * | (n = 1) |
| 18 | * | (n = 3) |
| 19 | * | (n = 3) |
| 20 | * | (n = 3) |
| 21 | * | (n = 3) |
| 22 | * | (n = 1) |
| 23 | * | (n = 1) |
| 24 | * | (n = 1) |
| 25 | * | (n = 1) |
| 26 | * | (n = 3) |
| 27 | * | (n = 1) |
| 28 | * | (n = 3) |
| 29 | * | (n = 1) |
| 30 | * | (n = 1) |
| 31 | * | (n = 1) |
| 32 | * | (n = 3) |
| 32 | * | (n = 3) |
| 35 | * | (n = 1) |

### Example 100. DPP1 Cell Single-Point Percent Inhibition Testing

HL-60 cells (ATCC; Manassas, VA) were maintained in RPMI-1640 supplemented with 20% heat-inactivated FBS and 1× Antibiotic Antimycotic (Cytiva; Marlborough, MA). Media was changed every three to four days and cells were not allowed to exceed 1×10⁶ cells per mL. Prior to assay, cells were collected by centrifugation at 500 rcf for 3 minutes, resuspended in RPMI (with no additional supplements) and counted. Cells were diluted in RPMI to a concentration of 5×10⁵ live cells per mL and transferred to black 96-well plates for assay, 60 μL per well. Test articles were diluted to 25 nM in RPMI plus 0.5% DMSO, and 20 μL was added to each assay well. Compound was allowed to pre-incubate with cells with gentle shaking at 100 rpm for 60 minutes at 37° C. in a cell culture incubator maintained at 5% CO₂, after which 20 μL of 500 μM H-Gly-Phe-AFC cell-permeable substrate (MP Biomedicals; Solon, OH) in RPMI was added to each well, giving final compound concentration of 5 nM, a final substrate concentration of 100 μM and a final DMSO concentration of 0.1%. Plates were returned to the incubator with shaking at 100 rpm for 30 minutes, after which fluorescence was measured at Excitation/Emission 400/505 nm. Percent Inhibition was calculated from RFU values by first subtracting background values (wells in which RPMI was substituted for cells) from each well, and then subtracting background-subtracted RFU derived from wells that received a final concentration of 10 μM brensocatib. This value represents the non-specific substrate cleavage from the HL-60 cells, and the remaining RFU should theoretically be solely the contribution of DPP1 enzyme activity. The final percent inhibition value was calculated for each test article based on remaining RFU compared to RFU from wells that received only vehicle control.

Results are provided in Table 6 below. In Table 6, *** represents percent inhibition of ≥93.9%, ** represents percent inhibition in the range of 76.5-93.9%, and * represents percent inhibition of ≤76.5%.

TABLE 6
DPP1 Cell Single-Point Percent Inhibition (n = 1)

| Comp. ID | % Inhibition |
|---|---|
| 18 | * |
| 19 | * |
| 36 | * |
| 37 | * |
| 38 | * |
| 39 | * |
| 40 | * |
| 41 | * |
| 42 | * |
| 43 | * |
| 44 | * |
| 45 | * |
| 46 | * |
| 47 | * |
| 48 | * |
| 49 | * |
| A11 | * |
| A80 | ** |
| A81 | * |
| A82 | ** |
| A83 | ** |
| A84 | * |
| A85 | * |
| A86 | * |
| A87 | ** |
| A88 | ** |
| A90 | * |
| A91 | * |
| A92 | * |
| A93 | * |
| A94 | * |
| A95 | * |
| A96 | * |
| A97 | * |
| A98 | * |
| A101 | ** |
| A104 | * |
| A105 | * |
| A106 | * |
| A107 | * |
| A108 | * |
| A109 | * |
| A110 | * |
| A111 | * |
| A112 | * |
| A113 | * |
| A114 | * |
| A115 | * |
| A116 | * |
| A117 | * |
| A118 | * |
| A119 | * |
| A120 | * |
| A122 | ** |
| A123 | *** |
| A125 | *** |
| A126 | *** |
| A128 | ** |
| A129 | ** |
| A131 | * |
| A132 | ** |
| A134 | * |
| A135 | * |
| A137 | ** |
| A138 | ** |
| A140 | ** |
| A141 | ** |
| A142 | * |
| A144 | * |
| B17 | * |
| B20 | *** |
| B21 | ** |
| B24 | * |
| B25 | * |
| B26 | * |
| B28 | * |
| B29 | * |
| B30 | * |
| B31 | ** |
| B32 | ** |
| B33 | * |
| B34 | * |
| C8 | * |
| C11 | ** |
| C14 | * |
| C16 | * |
| C17 | * |
| C19 | * |
| C20 | * |
| C22 | * |
| C23 | * |
| C25 | ** |
| C26 | * |
| C28 | ** |
| C29 | * |
| C31 | * |
| C32 | * |
| F5 | * |
| F6 | * |
| F7 | * |
| F8 | * |
| F9 | * |
| F10 | * |
| F11 | * |
| F12 | * |
| F14 | * |
| F15 | * |
| F16 | * |
| F17 | * |
| F18 | * |
| F19 | * |
| F20 | * |
| F21 | * |
| F22 | * |
| F23 | * |
| F24 | * |
| F25 | * |
| F26 | * |
| F27 | * |
| F28 | * |
| F29 | * |
| F30 | * |
| G1 | * |
| G2 | * |
| G3 | ** |
| G4 | *** |
| G5 | * |
| G6 | * |
| G8 | * |
| G9 | * |
| G10 | * |
| G11 | * |
| G12 | * |
| H3 | * |
| H4 | * |
| H5 | ** |
| H6 | * |
| H8 | * |
| H9 | * |
| H10 | * |
| H11 | ** |
| H12 | * |
| H13 | ** |
| H14 | ** |
| H15 | * |
| H16 | * |
| H17 | * |
| H18 | * |
| H19 | * |
| H20 | * |
| H21 | * |
| H24 | * |
| H27 | * |
| H28 | * |
| H29 | * |
| H30 | * |
| H31 | * |
| H32 | * |
| H33 | * |
| H34 | * |
| H35 | * |
| H36 | * |
| H37 | * |
| H38 | * |
| H39 | * |
| H40 | * |
| H41 | * |
| H42 | * |
| H43 | * |
| H44 | * |
| I2 | * |
| I3 | * |
| I8 | * |

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1.-309. (canceled)

310. A compound of formula (VI) 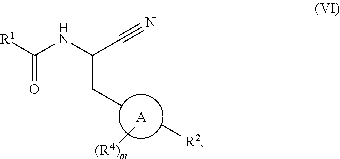
or a pharmaceutically acceptable salt, a stereoisomer, or a deuterated form thereof wherein:
ring A is a carbocyclyl, aryl, heterocyclyl, or heteroaryl ring;
R¹ is 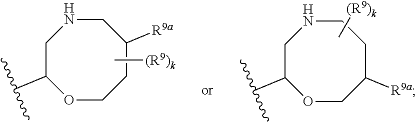
R² is phenyl, wherein R² is optionally substituted with 1, 2, 3, or 4 R⁶, each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R₈, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, or —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
R^{9a} is —O—(C₁-C₆ alkyl);
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
m is 0, 1, 2, or 3; and
k is 0, 1, 2, or 3;
wherein the compound is not 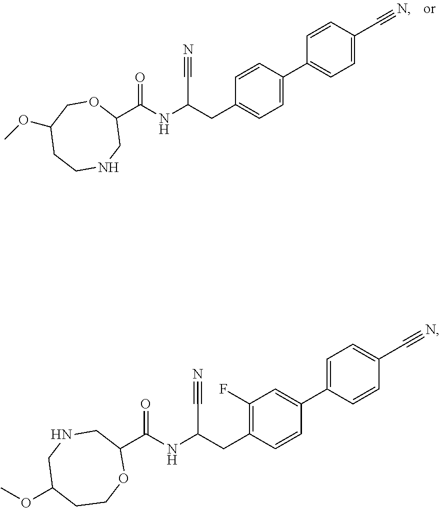 [SMILES: COC1CCNCC(C(=O)NC(C#N)Cc2ccc(-c3ccc(C#N)cc3)cc2)OC1 ; COC1CCOC(C(=O)NC(C#N)Cc2ccc(-c3ccc(C#N)cc3)cc2F)CNC1]
or a pharmaceutically acceptable salt or a stereoisomer thereof.

311. The compound of claim 310, or a pharmaceutically acceptable salt, a stereoisomer, or a deuterated form thereof, wherein ring A is a phenyl or heteroaryl ring.

312. The compound of claim 310, or a pharmaceutically acceptable salt, a stereoisomer, or a deuterated form thereof, wherein ring A is

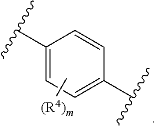

313. The compound of claim 310, or a pharmaceutically acceptable salt, a stereoisomer, or a deuterated form thereof, wherein ring A is

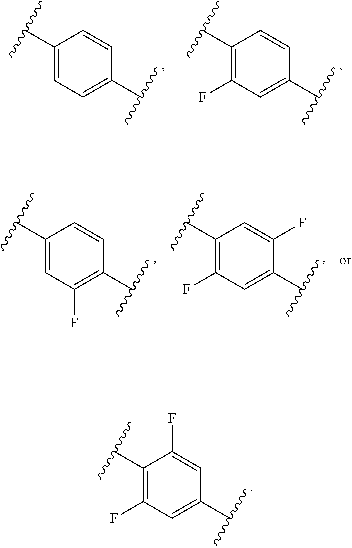 [SMILES: *c1ccc(*)cc1 ; *c1ccc(*)c(F)c1 ; *c1ccc(*)c(F)c1 ; *c1cc(F)c(*)cc1F ; *c1cc(F)c(*)c(F)c1]

314. The compound of claim 310, or a pharmaceutically acceptable salt, a stereoisomer, or a deuterated form thereof, wherein m is 0 or 1.

315. The compound of claim 310, or a pharmaceutically acceptable salt, a stereoisomer, or a deuterated form thereof, wherein each R⁴ is independently halogen, —C₁-C₃ alkyl, or —CN.

316. The compound of claim 310, or a pharmaceutically acceptable salt, a stereoisomer, or a deuterated form thereof, wherein m is 1 or 2 and R⁴ is each independently a halogen.

317. The compound of claim 310, or a pharmaceutically acceptable salt, a stereoisomer, or a deuterated form thereof, wherein R¹ is 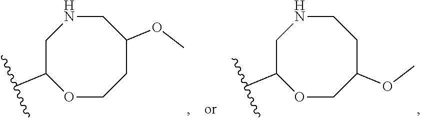 [SMILES: *C1CNCC(OC)CCO1 ; *C1CNCCC(OC)CO1]
each of which is optionally substituted with 1, 2, or 3 R⁹.

318. The compound of claim 310, or a pharmaceutically acceptable salt, a stereoisomer, or a deuterated form thereof, wherein R¹ is

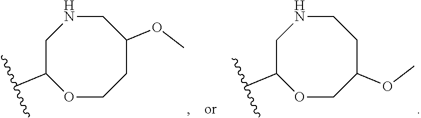 [SMILES: *C1CNCC(OC)CCO1 ; *C1CNCCC(OC)CO1]

319. The compound of claim 310, or a pharmaceutically acceptable salt, a stereoisomer, or a deuterated form thereof, wherein R¹ is

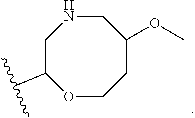 [SMILES: *C1CNCC(OC)CCO1]

320. The compound of claim 310, or a pharmaceutically acceptable salt, a stereoisomer, or a deuterated form thereof, wherein R¹ is

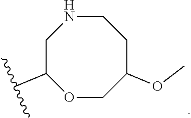 [SMILES: *C1CNCCC(OC)CO1]

321. The compound claim 310, or a pharmaceutically acceptable salt, a stereoisomer, or a deuterated form thereof, wherein R² is

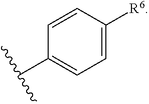

322. The compound of claim 321, or a pharmaceutically acceptable salt, a stereoisomer, or a deuterated form thereof, wherein R⁶ is independently halogen, —OH, —CN, —SO₂CH₃,

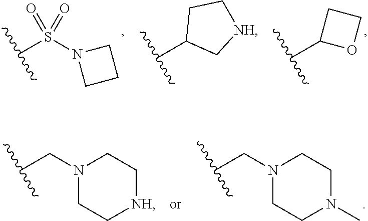

323. The compound of claim 310, or a pharmaceutically acceptable salt, a stereoisomer, or a deuterated form thereof, wherein R² is

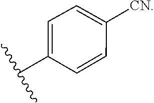 [SMILES: *c1ccc(C#N)cc1]

324. The compound of claim 310, or a pharmaceutically acceptable salt, a stereoisomer, or a deuterated form thereof, wherein R² is

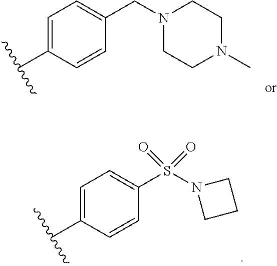 [SMILES: *c1ccc(CN2CCN(C)CC2)cc1 ; *c1ccc(S(=O)(=O)N2CCC2)cc1]

325. The compound of claim 310, or a pharmaceutically acceptable salt, a stereoisomer, or a deuterated form thereof, wherein each R⁶ is independently halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂NH₂, —SO₂NH(C₁-C₃ alkyl), —SO₂N(C₁-C₃ alkyl)₂, 4-6 membered heterocycle, or —(C₁-C₆ alkylene)-(4-6 membered heterocycle).

326. The compound of claim 310, or a pharmaceutically acceptable salt, a stereoisomer, or a deuterated form thereof, wherein each R⁶ is independently halogen, —OH, —CN, —SO₂CH₃,

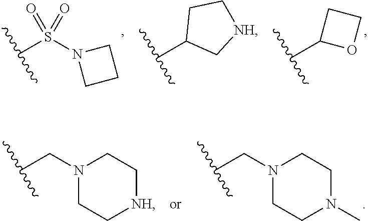

327. The compound of claim 310, or a pharmaceutically acceptable salt, a stereoisomer, or a deuterated form thereof, wherein R^{9a} is —O—(C₁-C₃ alkyl).

328. The compound of claim 310, or a pharmaceutically acceptable salt, a stereoisomer, or a deuterated form thereof, wherein R^{9a} is —OCH₃.

329. The compound of claim 310, or a pharmaceutically acceptable salt, a stereoisomer, or a deuterated form thereof, wherein each R⁹ is independently halogen, —C₁-C₃ alkyl, —OH, —OCH₃, —OCH₂CH₃, —OCD₃, —OCF₃, or —OCHF₂.

330. The compound of claim 310, or a pharmaceutically acceptable salt, a stereoisomer, or a deuterated form thereof, wherein each R⁹ is independently —OH, —OCH₃, or methyl.

331. The compound of claim 310, or a pharmaceutically acceptable salt, a stereoisomer, or a deuterated form thereof, wherein each R¹⁰ is independently —C₁-C₆ alkyl, —CH₂CH₂OCH₂CH₃, —CH₂CH₂OCH₂CH₂CH₃, —CH₂CH₂OCH₂CH₂CH₂CH₃, —CH₂CH₂OCH₂CH₂OH, —CH₂CH₂OCH₂CH₂OCH₃, —CH₂CH₂N(CH₃)₂, or

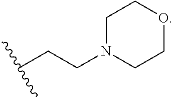 [SMILES: *CCN1CCOCC1]

332. The compound of claim 310, or a pharmaceutically acceptable salt, a stereoisomer, or a deuterated form thereof, wherein:
ring A is 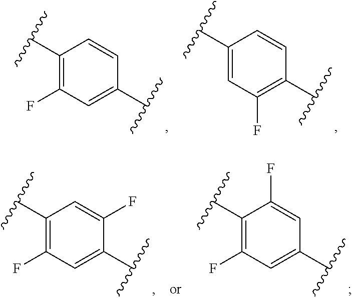
R¹ is 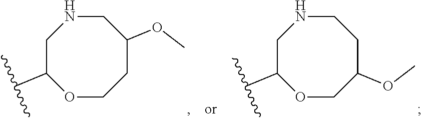 [SMILES: *C1CNCC(OC)CCO1 ; *C1CNCCC(OC)CO1]
R² is 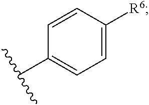
and
R⁶ is halogen, —OH, —CN—C₁-C₃ alkyl, —C₁-C₃ haloalkyl, —S(C₁-C₃ alkyl), —SO(C₁-C₃ alkyl), —SO₂(C₁-C₃ alkyl), —SO₂NH₂, —SO₂NH(C₁-C₃ alkyl), —SO₂N(C₁-C₃ alkyl)₂, 4-6 membered heterocycle, or —(C₁-C₆ alkylene)-(4-6 membered heterocycle).

333. The compound of claim 332, or a pharmaceutically acceptable salt, a stereoisomer, or a deuterated form thereof, wherein R⁶ is independently halogen, —OH, —CN, —SO₂CH₃,

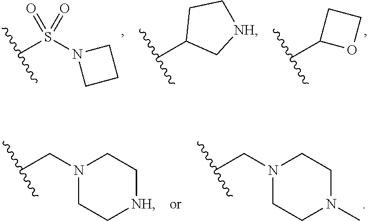

334. A compound of formula (VI) 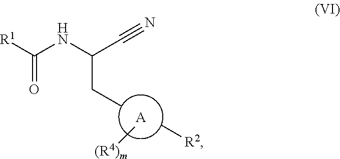
or a pharmaceutically acceptable salt, a stereoisomer, or a deuterated form thereof wherein:
ring A is a carbocyclyl, aryl, heterocyclyl, or heteroaryl ring;
R¹ is 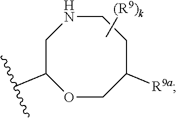
R² is phenyl, wherein R² is optionally substituted with 1, 2, 3, or 4 R⁶, each R⁴ is independently halogen, —C₁-C₆ alkyl, —C₁-C₆ haloalkyl, —S(C₁-C₆ alkyl), —SO(C₁-C₆ alkyl), —SO₂(C₁-C₆ alkyl), or —CN;
each R⁶ is independently halogen, —OH, oxo, —CN, —C₁-C₆ alkyl, —C₁-C₆ alkyl-OH, —O—(C₁-C₆ alkyl), —(C₁-C₆ alkylene)-O—(C₁-C₆ alkyl), —(C₁-C₆ alkylene)-O—(C₁-C₆ alkyl)-OH, —(C₁-C₆ alkylene)-O—(C₁-C₆ alkylene)-O—(C₁-C₆ alkyl), —(C₁-C₆ alkylene)-O—(C₁-C₆ alkylene)-O—(C₁-C₆ alkyl)-OH, —C₁-C₁₀ haloalkyl, —C₁-C₁₀ haloalkyl-OH, —NR⁷R⁸, —C₁-C₆ alkyl-NR⁷R⁸, —SF₅, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle, wherein each carbocycle and heterocycle is optionally substituted by 1 or 2 R¹⁰;
R⁷ and R⁸ are each independently hydrogen, C₁₋₆ alkyl, or —(C₁-C₆ alkylene)-O—(C₁-C₆ alkyl), or R⁷ and R⁸ together with the nitrogen atom to which they are attached form a heterocyclyl;
each R⁹ is independently halogen, oxo, —CN, —OH, —NH₂, —NH(C₁-C₆ alkyl), —N(C₁-C₆ alkyl)₂, —COOH, —C₁-C₆ alkyl, —C₁-C₆ alkyl-OH, —CONH₂, —SH, —S(═O)NH₂, —S(O)₂NH₂, —OC₁-C₆ alkyl, halogenated —OC₁-C₆ alkyl, —S(C₁-C₆ alkyl), —SO(C₁-C₆ alkyl), —SO₂(C₁-C₆ alkyl), —SO₂N(C₁-C₆ alkyl), —SO₂NR⁷R⁸, —S(═NH)(O)(C₁-C₆ alkyl), —(C₁-C₆ alkylene)-carbocyclyl, or —(C₁-C₆ alkylene)-heteroaryl;
R^{9a} is —O—(C₁-C₆ alkyl);
each R¹⁰ is independently halogen, —OH, oxo, —CN, —C₁-C₆ alkyl, —C₁-C₆ alkyl-OH, —C₁-C₁₀ haloalkyl, —C₁-C₁₀ haloalkyl-OH, —NR⁷R⁸, —C₁-C₆ alkyl-NR⁷R⁸, —S(C₁-C₆ alkyl), —SO(C₁-C₆ alkyl), —SO₂(C₁-C₆ alkyl), —SO₂N(C₁-C₆ alkyl), —SO₂NR⁷R⁸, —(C₁-C₆ alkylene)-carbocycle, —(C₁-C₆ alkylene)-heterocycle, carbocycle, or heterocycle;
m is 1, 2, or 3; and
k is 0, 1, 2, or 3.

335. The compound of claim 334, or a pharmaceutically acceptable salt, a stereoisomer, or a deuterated form thereof, wherein ring A is

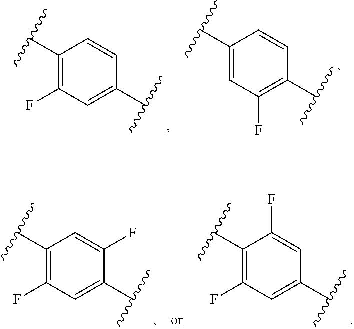 [SMILES: *c1ccc(*)c(F)c1 ; *c1ccc(*)c(F)c1 ; *c1cc(F)c(*)cc1F ; *c1cc(F)c(*)c(F)c1]

336. The compound of claim 334, or a pharmaceutically acceptable salt, a stereoisomer, or a deuterated form thereof, wherein R² is

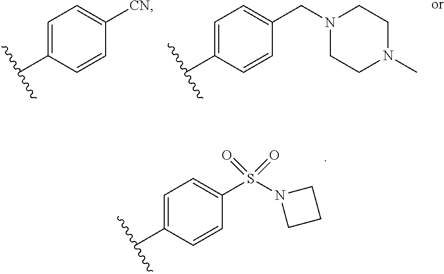 [SMILES: *c1ccc(C#N)cc1 ; *c1ccc(CN2CCN(C)CC2)cc1 ; "or" ; *c1ccc(S(=O)(=O)N2CCC2)cc1]

337. The compound of claim 310, or a pharmaceutically acceptable salt, a stereoisomer, or a deuterated form thereof, wherein the compound is selected from: 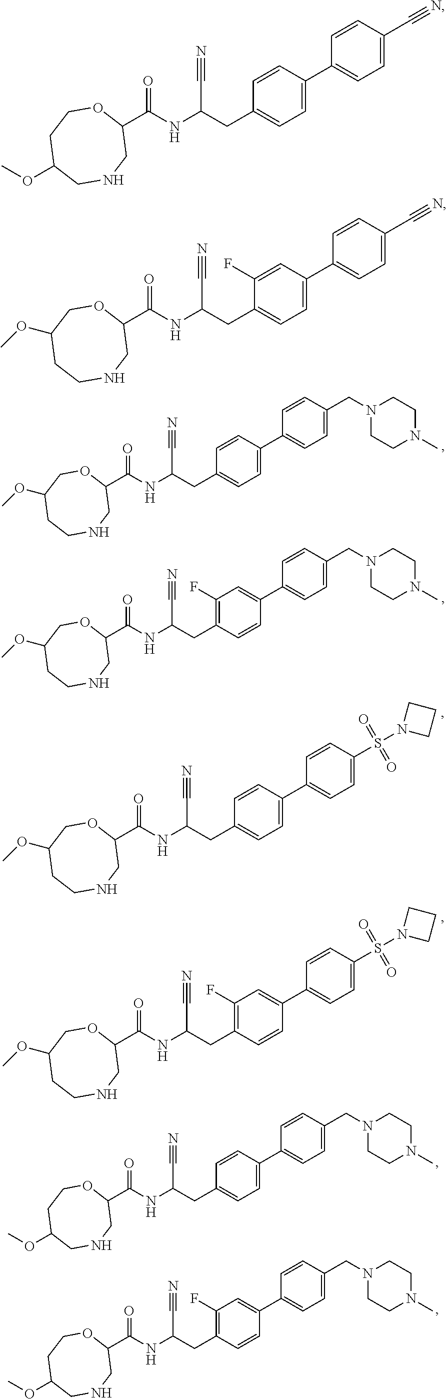 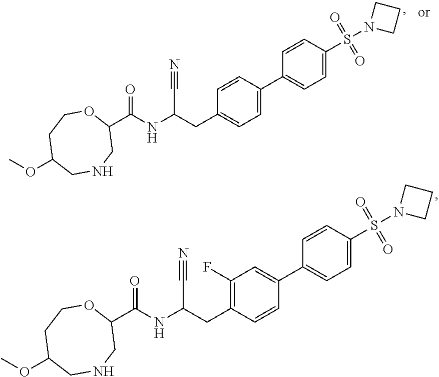 [SMILES: COC1CCOC(C(=O)NC(C#N)Cc2ccc(-c3ccc(S(=O)(=O)N4CCC4)cc3)cc2)CNC1 ; COC1CCOC(C(=O)NC(C#N)Cc2ccc(-c3ccc(S(=O)(=O)N4CCC4)cc3)cc2F)CNC1]
or a pharmaceutically acceptable salt, a stereoisomer, or a deuterated form thereof.

338. The compound of claim 310, or a pharmaceutically acceptable salt, a stereoisomer, or a deuterated form thereof, wherein the compound is selected from: 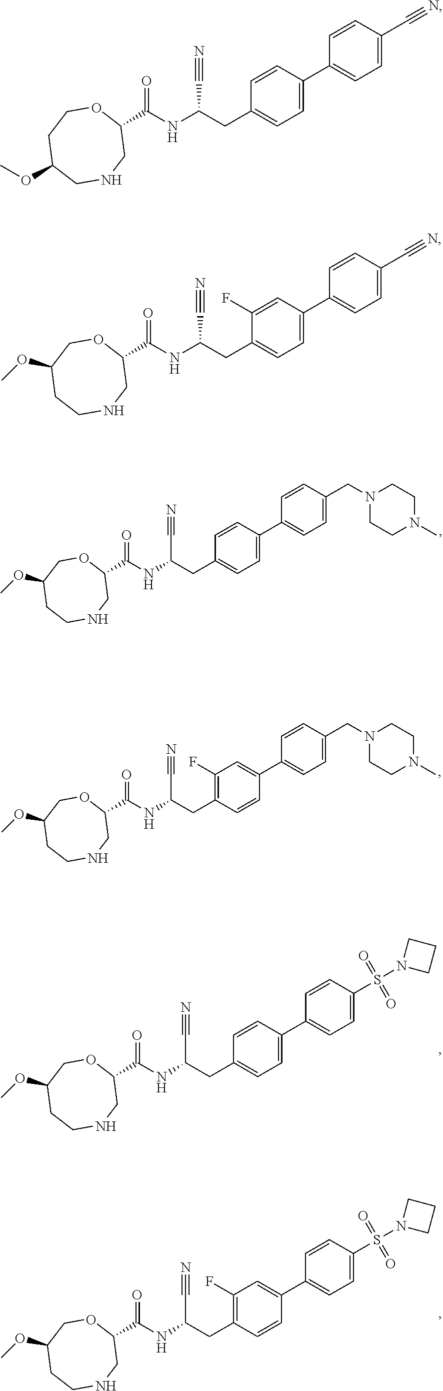 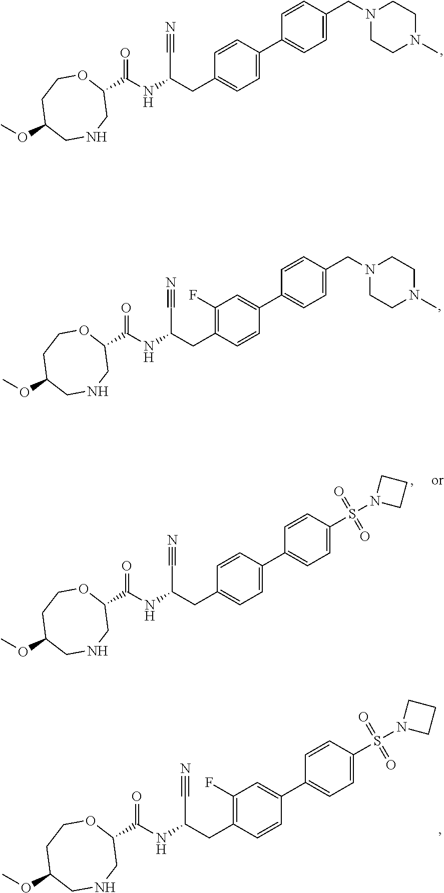
or a pharmaceutically acceptable salt or a deuterated form thereof.

339. A pharmaceutical composition comprising an effective amount of a compound of claim 310, or a pharmaceutically acceptable salt or deuterated form thereof and a pharmaceutically acceptable adjuvant, diluent or carrier.
